SEQUENCE LISTING (10 sequences)

SEQ ID NO: 1 - protein, 10 aa, Artificial Sequence
  Description of Artificial Sequence Synthetic peptide
DRVYIHPFHL

SEQ ID NO: 2 - protein, 8 aa, Artificial Sequence
  Description of Artificial Sequence Synthetic peptide
DRVYIHPF

SEQ ID NO: 3 - protein, 9 aa, Artificial Sequence
  Description of Artificial Sequence Synthetic peptide
DRVYIHPFH

SEQ ID NO: 4 - protein, 595 aa, Homo sapiens
STIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPY

SEQ ID NO: 5 - protein, 587 aa, Homo sapiens
VTDEAEASKFVEEYDRTSQVVWNEYAEANWNYNTNITTETSKILLQKNMQIANHTLKYGTQARKFDVNQLQNTTIKRIIKKVQDLERAALPAQELEEYNKILLDMETTYSVATVCHPNGSCLQLEPDLTNVMATSRKYEDLLWAWEGWRDKAGRAILQFYPKYVELINQAARLNGYVDAGDSWRSMYETPSLEQDLERLFQELQPLYLNLHAYVRRALHRHYGAQHINLEGPIPAHLLGNMWAQTWSNIYDLVVPFPSAPSMDTTEAMLKQGWTPRRMFKEADDFFTSLGLLPVPPEFWNKSMLEKPTDGREVVCHASAWDFYNGKDFRIKQCTTVNLEDLVVAHHEMGHIQYFMQYKDLPVALREGANPGFHEAIGDVLALSVSTPKHLHSLNLLSSEGGSDEHDINFLMKMALDKIAFIPFSYLVDQWRWRVFDGSITKENYNQEWWSLRLKYQGLCPPVPRTQGDFDPGAKFHIPSSVPYIRYFVSFIIQFQFHEALCQAAGHTGPLHKCDIYQSKEAGQRLATAMKLGFSRPWPEAMQLITGQPNMSASAMLSYFKPLLDWLRTENELHGEKLGWPQYNWTPN

SEQ ID NO: 6 - protein, 587 aa, Homo sapiens; identical to SEQ ID NO: 5

SEQ ID NO: 7 - protein, 9 aa, Artificial Sequence
  Description of Artificial Sequence Synthetic peptide
RPPGFSPFR

SEQ ID NO: 8 - protein, 5 aa, Artificial Sequence
  Description of Artificial Sequence Synthetic peptide
YGGFL

SEQ ID NO: 9 - protein, 5 aa, Artificial Sequence
  Description of Artificial Sequence Synthetic peptide
YGGFM

SEQ ID NO: 10 - protein, 5 aa, Artificial Sequence
  Description of Artificial Sequence Synthetic peptide
DRVYI

(19) United States
(12) Patent Application Publication
(10) Pub. No.: US 2004/0209344 A1
(43) Pub. Date: Oct. 21, 2004

(54) Crystal structure of angiotensin-converting enzyme-related carboxypeptidase

(76) Inventors: Michael W. Pantoliano, Boxford, MA (US); M. Dominic Ryan, Littleton, MA (US); Bart Lee Staker, Kingston, WA (US); G. Sridhar Prasad, San Diego, CA (US); Jin Tang, Canton, MA (US); Saurabh Prabhakar Menon, Medford, MA (US); Paul S. Towler, Gloucester, MA (US); David H. Williams, London (GB); Martin Fisher, Wakefield (GB)

Correspondence Address: FISH & NEAVE, 1251 AVENUE OF THE AMERICAS, 50TH FLOOR, NEW YORK, NY, 10020-1105, US

(21) Appl. No.: 10/659,000

(22) Filed: Sep. 9, 2003

Related U.S. Application Data
(60) Provisional application No. 60/410,010, filed on Sep. 9, 2002.

(51) Int. Cl. C12N 9/64; G06F 19/00; G01N 33/48; G01N 33/50

(52) U.S. Cl. 435/226; 702/19

(57) ABSTRACT

The invention relates to molecules or molecular complexes which comprise binding pockets of angiotensin-converting enzyme-related carboxypeptidase or its homologues. The invention relates to a computer comprising a data storage medium encoded with the structure coordinates of such binding pockets. The invention also relates to methods of using the structure coordinates to solve the structure of homologous proteins or protein complexes. The invention relates to methods of using the structure coordinates to screen for and design compounds that bind to angiotensin-converting enzyme-related carboxypeptidase protein or homologues thereof. The invention also relates to crystallizable compositions and crystals comprising angiotensin-converting enzyme-related carboxypeptidase protein or angiotensin-converting enzyme-related carboxypeptidase protein complexes.

This application claims benefit of U.S. provisional application No. 60/377,510, filed Sep. 9, 2002, the disclosure of which is incorporated herein by reference.

### TECHNICAL FIELD OF INVENTION

The present invention relates to molecules or molecular complexes which comprise binding pockets of human angiotensin-converting enzyme-related carboxypeptidase (ACE2), or its homologues. The present invention provides a computer comprising a data storage medium encoded with the structure coordinates of such binding pockets. This invention also relates to methods of using the structure coordinates to solve the structure of homologous proteins or protein complexes. In addition, this invention relates to methods of using the structure coordinates to screen for and design compounds, including inhibitory compounds, that bind to ACE2 protein or homologues thereof. The invention also relates to crystallizable compositions and crystals comprising ACE2 protein or ACE2 protein complexes.

### BACKGROUND OF THE INVENTION

The angiotensin-converting enzyme-related carboxypeptidase (ACE2) has been recently discovered and characterized (Donoghue et al., Circ. Res. 87, pp. e1-e9 (2000); Tipnis et al., J. Biol. Chem. 275, pp. 33238-33243 (2000)). This large type I integral membrane enzyme of 805 residues is an anion activated zinc metalloenzyme that hydrolyzes amino acid residues from the C-terminus of oligopeptides. These catalytic characteristics are similar to those of its closest homologue, angiotensin-converting enzyme (ACE; E.C. 3.4.15.1), a dipeptidyl peptidase with which it shares about 42% sequence homology.

Two forms of ACE are found in humans, somatic ACE (sACE), observed in many tissues, and a germinal isoform of ACE localized to the testes (tACE). Somatic ACE, a large protein of 1306 residues, contains two tandem homologous catalytic species as a result of gene duplication (Soubrier et al., Proc. Natl. Acad. Sci. USA 85, pp. 9386-9390 (1988)). This duplication results in sACE having an N-terminal catalytic domain and a C-terminal catalytic domain in tandem, each of which has a separate zinc binding site (HEXXH motif).

Human germinal or testicular ACE (tACE), a smaller protein of 732 residues, contains a single catalytic domain, which is identical to the C-terminal domain of sACE (Ehlers et al., Proc. Natl. Acad. Sci. USA 86, pp. 7741-7745 (1989)). Human tACE, therefore, contains a single zinc binding site (HEXXH motif). Similarly, ACE2 contains just one zinc catalytic site (HEXXH motif).

Like ACE2, both somatic ACE and germinal ACE are type I integral membrane enzymes with their catalytic domains exposed on the exterior of the cells expressing them.

There are, however, significant differences in substrate specificity and inhibitor binding characteristics between ACE2 and ACE. These differences are reflected in the physiological differences observed in the phenotypes of knock out mice engineered to have loss of function of ACE (Krege et al., Nature, 375, pp. 146-148 (1995); Esther et al., Lab Invest., 74, pp. 953-965 (1996)) and/or ACE2 (Crackower et al., Nature, 417, pp. 822-828 (2002)).

First, in regard to enzymatic activity, ACE2 is a carboxypeptidase (Tipnis et al., supra; Donoghue et al., supra; Vickers et al., J. Biol. Chem., 277, pp. 14838-14843 (2002)), while ACE is a dipetidyl peptidase.

Angiotensin I (DRVYIHPFHL; SEQ ID NO: 1) is a substrate for both enzymes. ACE converts angiotensin I to the potent vasoconstrictor, angiotensin II (DRVYIHPF; SEQ ID NO: 2) and the dipeptide, HL. ACE2, however, converts angiotensin I to angiotensin 1-9 (DRVYIHPFH; SEQ ID NO: 3) and the amino acid, L. Interestingly, angiotensin II is also a substrate for ACE2 (Vickers et al., supra). Without being bound to theory, the fact that angiotensin II is also a substrate for ACE2 suggests that ACE2 may be involved in the inactivation of vasoconstriction peptides and acts in a compensatory role vis-à-vis ACE in the renin angiotensin system. Also, because of the central role that angiotensin II plays in regulating blood pressure, it has been suggested that ACE and ACE2 work together in systemic blood pressure homeostasis. However, the loss of ACE2 in knock out mice had no effect on blood pressure even in the presence of ACE inhibitors, although the hearts of ACE2 knock out mice were found to have cardiac dysfunction and up-regulation of hypoxia inducible factors. A biological role for ACE2 as an essential regulator of healthy heart function is therefore suggested by these loss of function studies. In this regard, potent and selective inhibitors of ACE2 (Dales et al., J. Am. Chem. Soc. 124, pp. 11852-11853 (2002)) have become available as additional tools for exploring the physiological role that ACE2 plays in healthy and diseased states, as well as drug candidates. A more comprehensive examination of the ACE2 and ACE literature may be found in recently published reviews (Turner and Hooper, Trends in Pharmacological Sci. 23, pp. 177-183 (2002); Danilczyk et al., J. Mol. Med. 81, pp. 227-234 (2003); Oudit et al., Trends Cardiovasc. Med. 13, pp. 93-101 (2003)).

An in vitro substrate profiling screen of 126 biological peptides identified just eleven peptides that are hydrolyzed by ACE2 (Vickers et al., supra). In every case, ACE2 was found to exhibit only carboxypeptidase activity. Of the seven best in vitro peptide substrates identified with kcat/Km>10⁵ M⁻¹ s⁻¹, proline or leucine was found to be the preferred residue in the P₁ position, and a hydrophobic residue was preferred in the P₁′ position, although basic residues at this position are also cleaved (dynorphin A 1-13, and neurotensin 1-8). Thus, a consensus prolyl carboxypeptidase activity has emerged from these substrate profiling studies for ACE2. Some of the best in vitro peptide substrates are: Apelin 13, des-Arg⁹ bradykinin, Angiotensin II, and Dynorphin A 1-13. The longest identified peptide substrate was apelin 36, a peptide of 36 residues (Vickers et al., supra).

The substrate specificity differences between ACE2 and ACE also translate into different inhibitor binding profiles. Potent ACE inhibitors such as captopril, lisinopril, and enalaprilat, which have been employed as anti-hypertensive drugs, did not inhibit ACE2 (Tipnis et al., supra). Conversely, potent ACE2 inhibitors weakly inhibit ACE (IC₅₀>10 μM) and carboxypeptidase A (CPA) (Dales et al., supra).

In the 46 years since the first isolation of ACE (Skeggs et al., J. Exp. Med. 103, pp. 295-299 (1956)), intensive research has led to the present understanding of the physiological role of ACE in the regulation of blood pressure and fluid and electrolyte balance in mammals (Inagami, Essays Biochem. 28, pp. 147-164 (1994)). However, the biological role of ACE2 appears distinct from that of ACE.

One way to further understand the substrate and inhibitor binding differences between ACE and ACE2 is through three-dimensional structural studies. The three-dimensional structure of the enzymes would also assist in the rational design of inhibitors, which can be drug candidates. Further, information provided by the X-ray crystal structure of ACE2-inhibitor complexes would be extremely useful in preparation of homology models of other ACE2 homologues. The determination of the amino acid residues in ACE2 binding pockets and the determination of the shape of those binding pockets would allow one to design inhibitors that bind more favorably to this entire class of enzymes.

Structures of proteins related to ACE2 have been reported in the Protein Data Bank (PDB) database (Berman et al., Nuc. Acids Res. 28, pp. 235-242 (2000)). These are: (A) the recently solved human tACE (Natesh et al., Nature 421, pp. 551-4 (2003)), an enzyme of the M2 metallopeptidase family (EC 3.4.15.1); (B) the Drosophila ACE structure (Kim et al., FEBS Letters 538, pp. 65-70 (2003)); and (C) the rat neurolysin (Brown et al., Proc. Natl. Acad. Sci. USA 98, pp. 3127-3132 (2001)), an M3 metallopeptidase family member (EC 3.4.24.16) with which ACE2 shares only about 17% sequence identity; and (D) the P. furiosus carboxypeptidase (Arndt et al., Structure 10, pp. 215-224 (2002)), a member of the M32 carboxypeptidase family.

### SUMMARY OF THE INVENTION

This invention provides for the first time the three-dimensional structure of the extracellular domains of human ACE2. That three-dimensional structure was determined by multiple isomorphous replacement with anomalous scattering (MIRAS) to 2.2 Å resolution. This invention also provides structures of human ACE2 with inhibitors bound at the active site. Those co-crystal structures were solved using molecular replacement methods. The present invention allows comparisons of human ACE2 and tACE structures to show the distinct and unique molecular features of the ACE2 structure.

The present invention also provides molecules or molecular complexes comprising ACE2 binding pockets, or ACE2-like binding pockets that have similar three-dimensional shapes. In one embodiment, the molecules or molecular complexes are ACE2 proteins, protein complexes or homologues thereof. In another embodiment, the molecules or molecular complexes are in crystalline form.

The invention provides crystallizable compositions and crystal compositions comprising human ACE2 or homologue thereof with or without a chemical entity. The invention provides a substantially pure human ACE2 protein. The invention also provides crystals of an ACE2 protein, protein complex, or homologues thereof.

The invention provides a computer comprising a machine-readable storage medium, comprising a data storage material encoded with machine-readable data, wherein the data defines the binding pocket or protein according to the structure coordinates of molecules or molecular complexes of ACE2 or ACE2-like proteins, or homologues thereof. The invention also provides a computer comprising the data storage medium. Such storage medium when read and utilized by a computer programmed with appropriate software can display, on a computer screen or similar viewing device, a three-dimensional graphical representation of such binding pockets. In one embodiment, the structure coordinates of said molecules or molecular complexes are produced by homology modeling of the coordinates of FIG. 1A, 2A, 3A or 3B.

The invention also provides methods for designing, selecting, evaluating and identifying and/or optimizing compounds which bind to the molecules or molecular complexes or their binding pockets. Such compounds are potential inhibitors of ACE2 or its homologues.

The invention also provides a method for determining at least a portion of the three-dimensional structure of molecules or molecular complexes which contain at least some structurally similar features to ACE2, particularly ACE2 homologues. This is achieved by using at least some of the structure coordinates obtained from the ACE2 protein or protein complexes.

### BRIEF DESCRIPTION OF THE FIGURES

The following abbreviations are used in FIGS. 1 and 2:

“Atom type” refers to the element whose coordinates are measured. The first letter in the column defines the element.

“Res” refers to the amino acid residue in the molecular model.

“X, Y, Z” define the atomic position of the element measured.

“B” is a thermal factor that measures movement of the atom around its atomic center.

“Occ” is an occupancy factor that refers to the fraction of the molecules in which each atom occupies the position specified by the coordinates. A value of “1” indicates that each atom has the same conformation, i.e., the same position, in the molecules.

FIG. 1A (1A-1 to 1A-100) lists the atomic coordinates for native human ACE2 (amino acid residues 19-740 of full-length human ACE2 protein (SEQ ID NO: 4) with residues 621-626 and 661-705 of full-length human ACE2 protein (SEQ ID NO: 4) built as alanines; residues 804-823 represent a section of residues which are built as alanines into the electron density and cannot be assigned exact amino acid numbers (residues 627 to 660 or residues 706 to 740 may include residues 804-823)) as derived from X-ray diffraction of the crystal before individual B-factor refinement. The coordinates are shown in Protein Data Bank (PDB) format. Residues NAG, TIP and ZN2 represent N-acetyl glucosamine (NAG) groups, water and zinc ion, respectively.

FIG. 2A (2A-1 to 2A-100) lists the atomic coordinates for native human ACE2 (amino acid residues 19-740 of full-length human ACE2 protein (SEQ ID NO: 4) with residues 621-626 and 661-705 of full-length human ACE2 protein (SEQ ID NO: 4) built as alanines; residues 804-823 represent a section of residues which are built as alanines into the electron density and cannot be assigned exact amino acid numbers (residues 627 to 660 or residues 706 to 740 may include residues 804-823)) as derived from X-ray diffraction of the crystal after individual B-factor refinement. The coordinates are shown in Protein Data Bank (PDB) format. Residues NAG, TIP and ZN2 represent N-acetyl glucosamine (NAG) groups, water and zinc ion, respectively.

FIG. 3A (3A-1 to 3A-89) lists the atomic coordinates for human ACE2 (amino acid residues 19-613 of full-length human ACE2 protein (SEQ ID NO: 4)) complexed with (S, S)2-{1-carboxy-2-[3-(3,5-dichloro-benzyl)-3H-imidazol-4-yl]-ethylamino}-4-methyl-pentanoic acid (inhibitor) as derived from X-ray diffraction of the crystal and refined to 3.3 Å resolution. The coordinates are shown in Protein Data Bank (PDB) format. Residues XX5, ZN. CL, and HOH represent inhibitor1, zinc ion, chloride ion and water, respectively.

FIG. 3B (3B-1 to 3B-95) lists the atomic coordinates for human ACE2 (amino acid residues 19-740 of full-length human ACE2 protein (SEQ ID NO: 4) with residues 621-626 and 661-705 of full-length human ACE2 protein (SEQ ID NO: 4) built as alanines; residues 804-823 represent a section of residues which are built as alanines into the electron density and cannot be assigned exact amino acid numbers (residues 627 to 660 or residues 706 to 740 may include residues 804-823)) complexed with (S, S) 2-{1-carboxy-2-[3-(3,5-dichloro-benzyl)-3H-imidazol-4-yl]-ethylamino}-4-methyl-pentanoic acid (inhibitor1) as derived from X-ray diffraction of the crystal and refined to 3.0 Å resolution. The coordinates are shown in Protein Data Bank (PDB) format. Residues NAG, TIP, XX5, ZN. And CL represent N-acetyl glucosamine (NAG) groups, water, inhibitor, zinc ion, and chloride ion, respectively.

FIG. 4 shows the primary sequence alignments for amino acid residues 19 to 613 of human ACE2 (full-length sequence: SwissProt Q9NRA7; SEQ ID NO: 4), the corresponding residues of the C-terminal catalytic domain of human somatic ACE (SEQ ID NO: 5) and the corresponding residues of germinal or testicular human ACE (tACE) (SEQ ID NO: 6; the numbering used for the tACE sequence follows Natesh et al., Nature 421, pp. 551-4 (2003)). The mature metallopeptidase domain of human ACE2 corresponds to residues 19 to 613. The Clustal W Alignment Tool (Higgins et al., Methods Enzymol. 266, pp. 383-402 (1996)) was used for these sequence alignments. The secondary structural elements of human ACE2 are denoted by ----->for helical sections and beta strands are denoted by -----●. Helices 1-3, 10-13 and 15, and beta strands 4-6 are found in subdomain I while helices 4-9, 14 and 16-23, and beta strands 1-3 and 7 are found in subdomain II. Residues which are identical between human ACE2, and human sACE and tACE are marked with an asterisk at the bottom of the sequences. The six predicted N-linked glycosylation sites for the metallopeptidase region of ACE2 are denoted by the strikethrough symbol, ▾. The beginning of the collectrin homology domain (Zhang et al., J. Biol. Chem. 276, pp. 17132-17139 (2001)) is denoted by the inverted triangle symbol, ▾. Zinc binding residues include: H374, H378, and E402(ACE2 sequence numbers given). Chloride ion binding residues include: R169, W477 and K481(ACE2 sequence numbers given) and additional chloride binding residues that occur for only sACE and tACE include Y224 and R522 (tACE sequence numbers given).

FIG. 5A depicts an overview of the overall fold of the native form of human ACE2. A schematic of the secondary structural elements of the native ACE2 structure at 2.2 Å resolution reveals and labels the 23 α-helix segments (cylinders) and the seven short beta structural elements (arrows). Subdomains I and II are labelled, and the C-terminus of the protein is marked as C⁶¹³.

FIG. 5B depicts a stereoview of the superposition of the native and inhibitor1-bound ACE2 structures and shows the 22° hinge bending movement of the subdomain I relative to subdomain II that occurs upon inhibitor binding to ACE2. In this figure, the top subdomains (subdomains II) of the native structure superimposes very closely to the top subdomain of the inhibitor1-bound ACE2 structure. The bottom subdomains (subdomains I) do not superimpose well due to the hinge bending movement. The lack of overlap between the structures is clearly shown in the first two N-terminal helices in the structures. The α1 and α2 helices of the native and inhibitor-bound ACE2 are labeled α1 and α2, and α1c and α2c, respectively. This figure shows the large difference in the positions of helices α1 and α2 in the native structure from the corresponding helices α1c and α2c in inhibitor1-bound ACE2 structures.

FIG. 6 depicts an overview (in stereo) of the two subdomains and hinge region of the native human ACE2 structure. The N-terminal and zinc containing subdomain is comprised of residues 19-102, 290-397, and 417-430 and is labeled subdomain I. The C-terminal subdomain is comprised of residues 103-289, 398-416, and 431-613 and is labeled subdomain II. Residues that lie on the hinge axis and involved in the ligand dependent hinge bending movement of the two subdomains are shown in light gray (including residues 99 to 100; 284 to 293; 396 to 397; 409 to 410; 433 to 434; 539 to 548; and 564 to 568). The zinc ion and the single bound chloride ion are shown as spheres. The zinc ion is the smaller sphere found in subdomain I.

FIG. 7 depicts experimental electron density map for inhibitor, (S, S)2-{1-carboxy-2-[3-(3,5-dichloro-benzyl)-3H-imidazol-4-yl]-ethylamino}-4-methyl-pentanoic acid, bound to human ACE2. The experimental electron density map represents 2|Fo|−|Fc| electron density contoured to 1.5 sigma. Good electron density can be seen for the inhibitor, (S, S)2-{1-carboxy-2-[3-(3,5-dichloro-benzyl)-3H-imidazol-4-yl]-ethylamino}-4-methyl-pentanoic acid, despite the lower resolution (3.3 Å) for the inhibitor-bound structure. Zinc ion is shown as a sphere.

FIG. 8A shows molecular surface representations of native human ACE2 structure generated using the default parameters of the program GRASP (Nicolls et al., Proteins: Struct. Func. Gen. 11, pp. 281-296 (1991)). Areas with positive or negative charge are shaded in gray. The left figure looks down into the deep active site cleft that separates the enzyme into two subdomains. The right figure is rotated 90° to show the profile along the length of the active site cleft.

FIG. 8B shows a molecular surface representation of a view of inhibitor-bound human ACE2 looking down the length of the active site tunnel. This figure is generated using the default parameters of the program GRASP (Nicolls et al., Proteins: Struct. Func. Gen. 11, pp. 281-296 (1991)). Areas with positive or negative charge are shaded in gray. The 3,5 dichlorobenzyl imidazole group of the inhibitor1 which fits into the S₁′ site of ACE2 can be seen through the small opening at the P, or leaving group end of the active site tunnel.

FIG. 9A shows a superposition of human ACE2 and human tACE (Natesh et al., supra) structures. The carbon-α traces of inhibitor1-bound ACE2 structure (using the coordinates of inhibitor1-bound ACE2 structure at refinement to 3.3 Å resolution given in FIG. 3A) and the lisinopril-bound tACE structure were superimposed using the program QUANTA (Molecular Simulations, Inc., San Diego, Calif. ©1998, 2000; Accelrys ©2001, 2002). The α-carbon atoms of all 588 amino acid residues of the tACE-lisinopril complex structure were superimposed onto the corresponding α-carbon atoms of the ACE-inhibitor1 structure using the program Molecular Operating Environment (MOE) (Chemical Computing Group, Inc., Montreal, Quebec Canada) to give an RMSD of 1.75 Å. Superposition of the 24 amino acid residues (including N149, A153, D269, W271, R273, F274, T276, N277, H345, P346, T347, A348, D367, T371, H374, E375, H378, E402, F504, H505, Y510, R514, Y515, and R518) within a 4.5 Å distance from inhibitor1 of the complex structure compared with the corresponding amino acid residues from the tACE-lisinopril structure yielded an RMSD of 1.14 Å. Zinc and chloride ions are shown as spheres (Cl⁻ ion is the larger sphere), and inhibitor1 is shown bound to the active site.

FIG. 9B shows a superposition of inhibitors bound to human ACE2 (using the coordinates of inhibitor1-bound ACE2 structure at refinement to 3.3 Å resolution given in FIG. 3A) and human tACE (Natesh et al., supra). Inhibitor1-bound ACE2 structure (FIG. 3A) is superimposed onto the lisinopril-bound tACE structure. The inhibitor and side chains of amino acid residues of the inhibitor1-bound ACE2 structure are shown as thicker stick representation, while the inhibitor and side chains of the amino acid residues of the lisinopril-bound tACE structure are shown in the thinner stick representation. Zinc and chloride ion 2 (CL2) of tACE are shown as spheres. Some residues worth noting that differ between ACE2 and tACE include: R273 (ACE2)->Q281 (tACE), F274 (ACE2)->T282 (tACE), Y510->V518 (tACE), D367->E376 (tACE). Residues derived from subdomain I have their α-backbone colored lighter gray, while residues derived from subdomain II have their α-backbone colored darker gray.

FIG. 10 shows a stereoview of the binding interactions for the inhibitor1-bound ACE2 complex (using the coordinates of inhibitor1-bound ACE2 structure at refinement to 3.3 Å resolution given in FIG. 3A). Residues of human ACE2 that contribute binding interactions to inhibitor1 are shown. These include R273 and H505, which are hydrogen bonded to the terminal carboxylate of the inhibitor; T371, which is hydrogen bonded to the imidazole ring of the dichlorobenzyl imidazole group of inhibitor1; the P346 carbonyl oxygen atom, which is hydrogen bonded to secondary amine group of the inhibitor; and F274 and H345, which form two sides of a hydrophobic lined tunnel for the dichlorobenzyl group of the inhibitor. Y515 and R514 are ˜3.8 and 4.1 Å, respectively, from the zinc-bound carboxylate group of inhibitor1. The zinc ion is shown as a smaller sphere.

FIG. 11 shows a schematic view of binding interactions for the inhibitor1-bound human ACE2 complex in stereo (using the coordinates of inhibitor1-bound ACE2 structure at refinement to 3.3 Å resolution given in FIG. 3A). Hydrogen bonding distances are given in angstroms (Å). Peptide binding subsites S₁ and S₁′ are labeled.

FIG. 12 shows a proposed five step mechanism for ACE2 catalyzed hydrolysis of peptide substrates using the coordinates of inhibitor1-bound ACE2 structure at refinement to 3.0 Å resolution given in FIG. 3B. Step 1: substrate binding to one subdomain that induces subdomain hinge movement to close the active site cleft and bring important residues into position for catalysis. Step 2: attack of zinc-bound water molecule at the carbonyl group of scissile bond to form tetrahedral intermediate and transfer of proton from attacking water to E375. Step 3: transfer of proton from E375 to leaving nitrogen atom of P₁′ residue. Step 4: final scissile bond breakage. Step 5: subdomain hinge bending movement to open active site cleft and release products.

FIG. 13 shows a diagram of a system used to carry out the instructions encoded by the storage medium of FIGS. 13 and 14.

FIG. 14 shows a cross section of a magnetic storage medium.

FIG. 15 shows a cross section of a optically-readable data storage medium.

### DETAILED DESCRIPTION OF THE INVENTION

In order that the invention described herein may be more fully understood, the following detailed description is set forth.

Throughout the specification, the word “comprise” or variations such as “comprises” or “comprising” will be understood to imply the inclusion of a stated integer or groups of integers but not the exclusion of any other integer or groups of integers.

The following abbreviations are used throughout the application:

| | |
|---|---|
| A = Ala = Alanine | T = Thr = Threonine |
| V = Val = Valine | C = Cys = Cysteine |
| L = Leu = Leucine | Y = Tyr = Tyrosine |
| I = Ile = Isoleucine | N = Asn = Asparagine |
| P = Pro = Proline | Q = Gln = Glutamine |
| F = Phe = Phenylalanine | D = Asp = Aspartic Acid |
| W = Trp = Tryptophan | E = Glu = Glutamic Acid |
| M = Met = Methionine | K = Lys = Lysine |
| G = Gly = Glycine | R = Arg = Arginine |
| S = Ser = Serine | H = His = Histidine |

As used herein, the following definitions shall apply unless otherwise indicated.

The term “about” when used in the context of RMSD values takes into consideration the standard error of the RMSD value, which is ±0.1 Å.

The term “ACE2 active site binding pocket” refers to a binding pocket of a molecule or molecular complex defined by the structure coordinates of a certain set of amino acid residues present in the ACE2 structure, as described below. This binding pocket is in an area in the ACE2 protein where the active site is located.

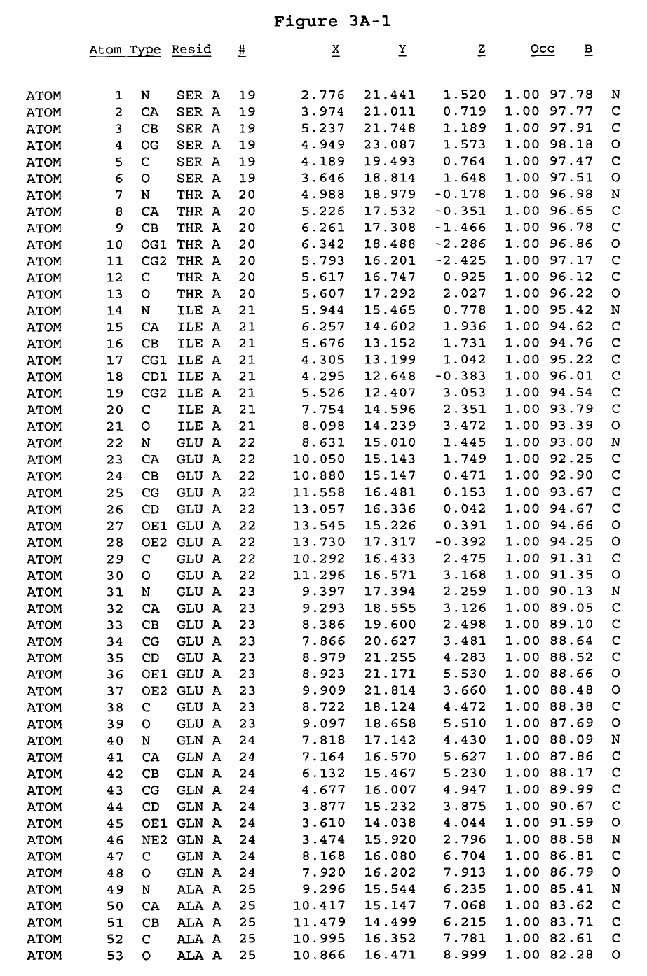

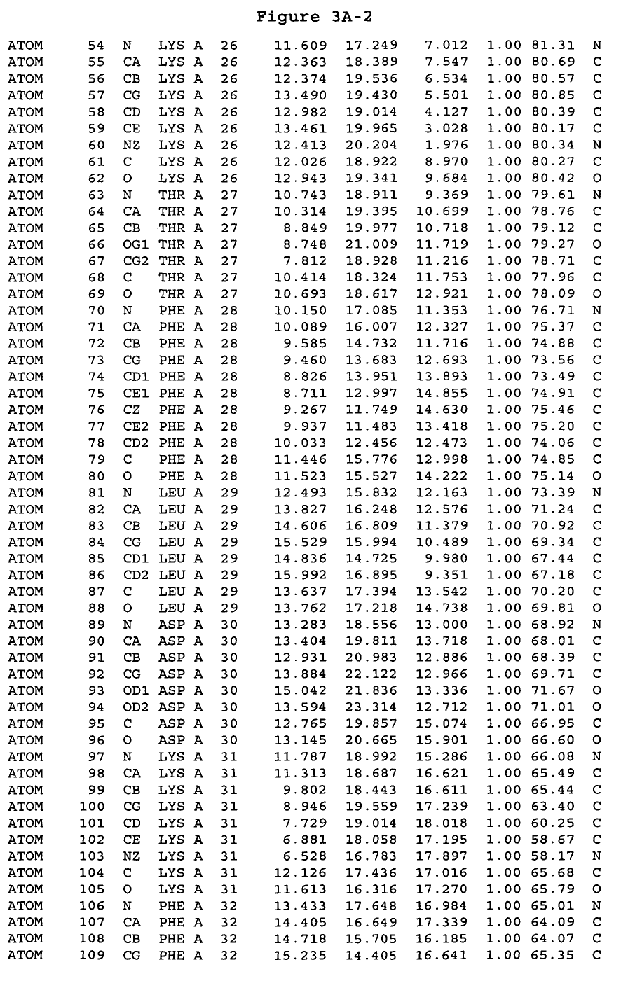

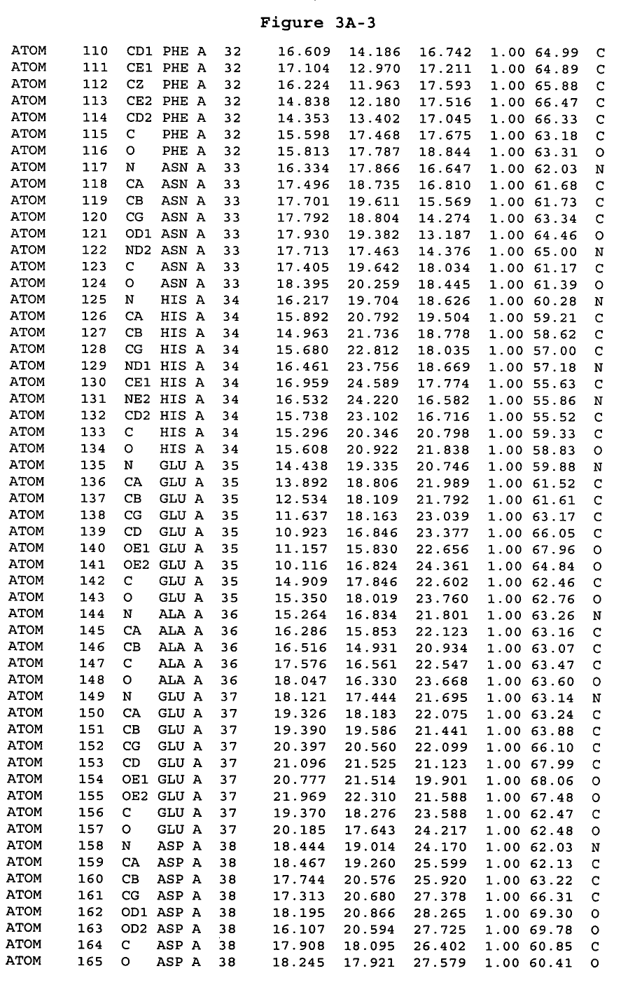

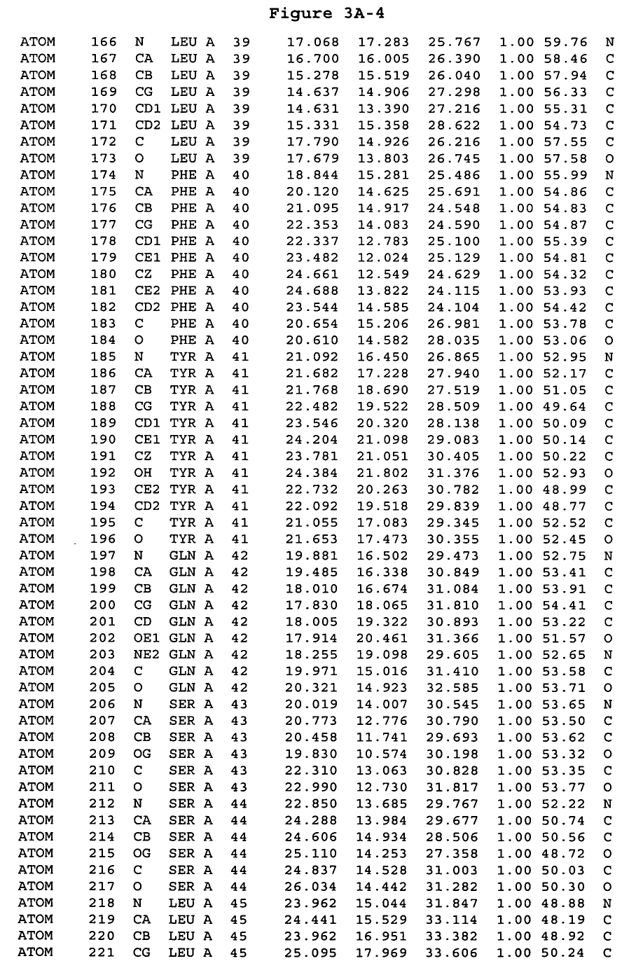

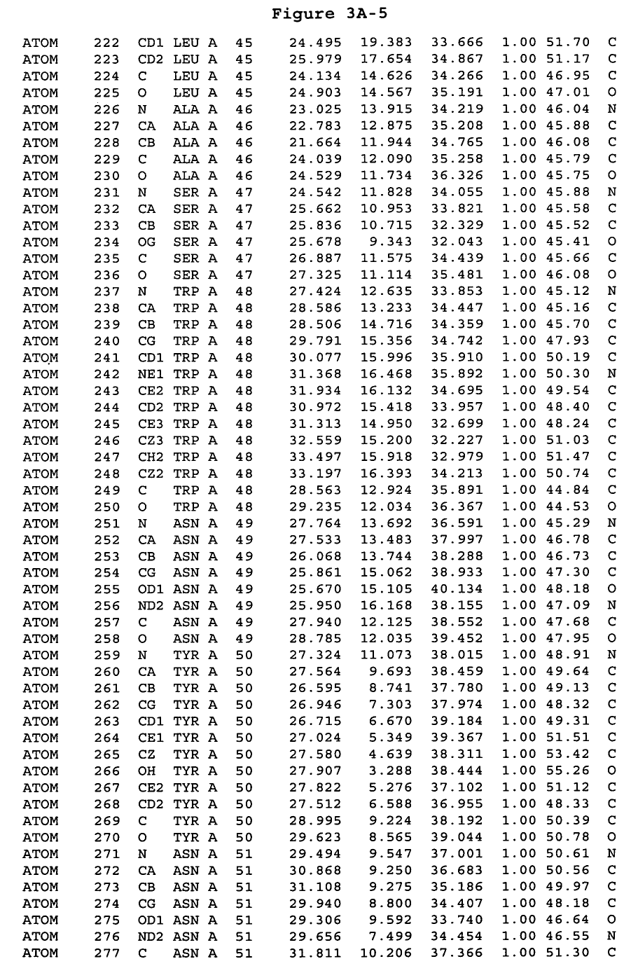

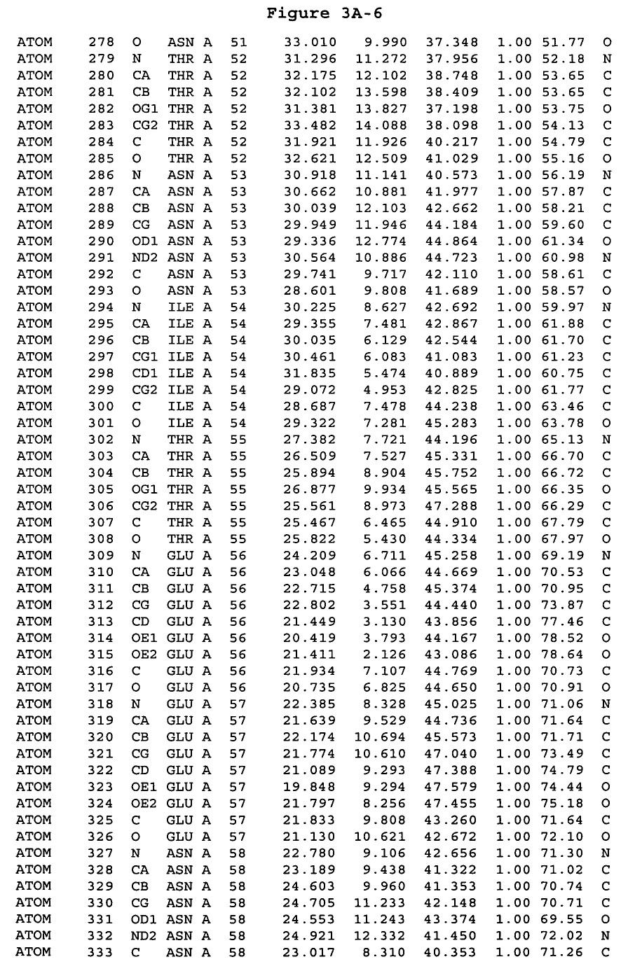

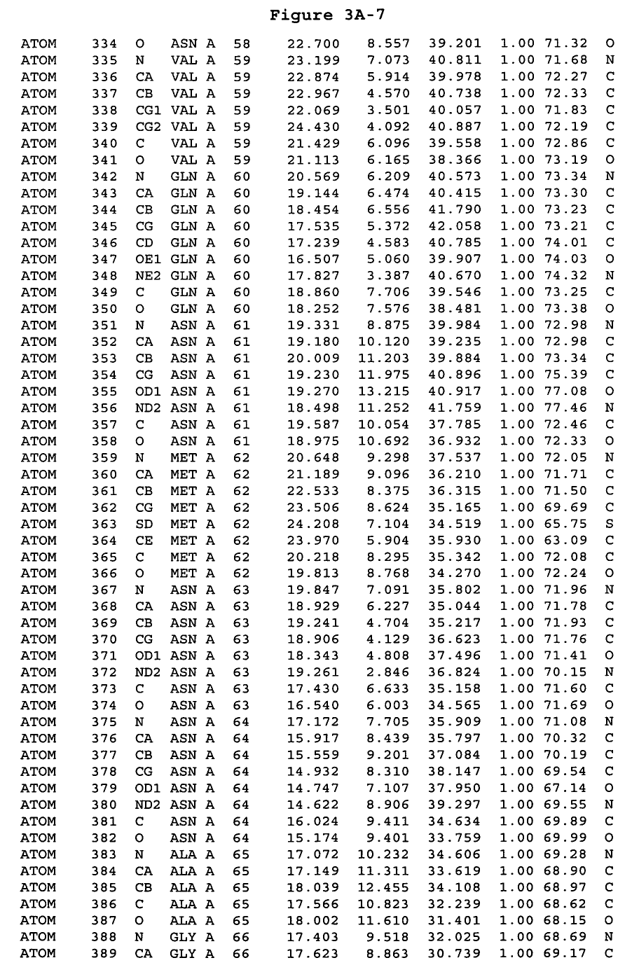

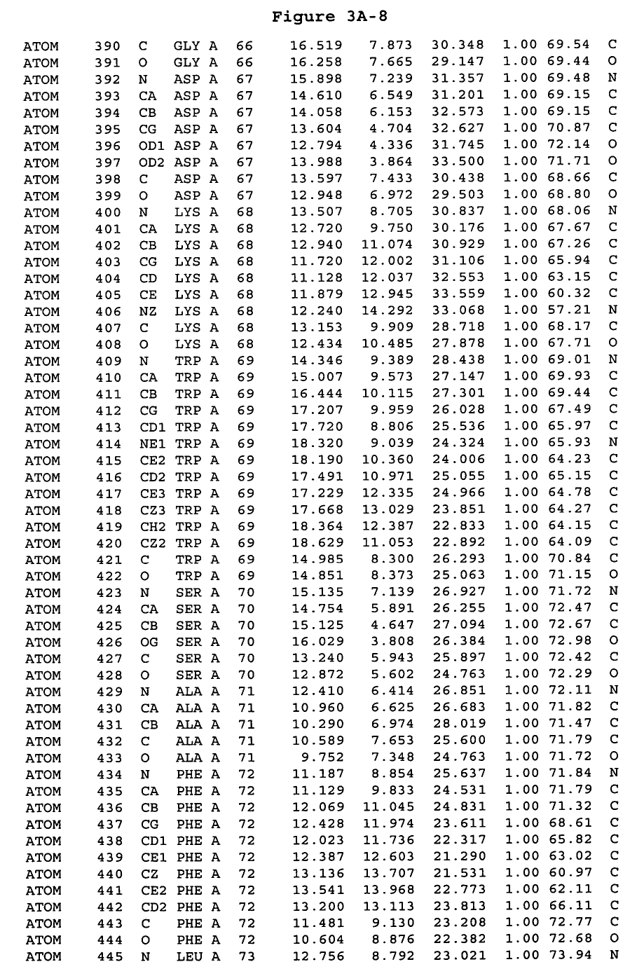

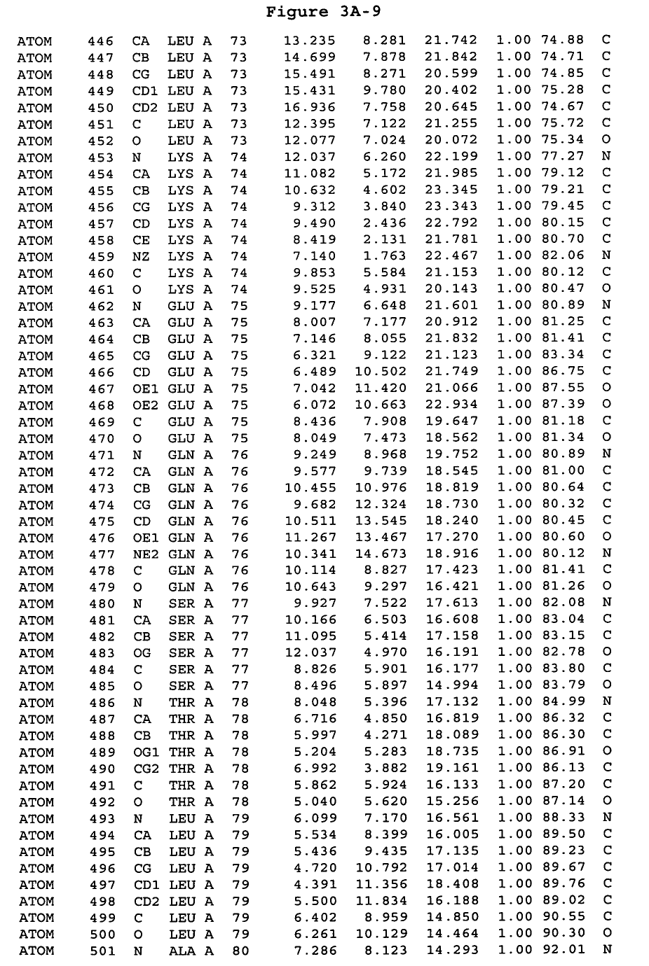

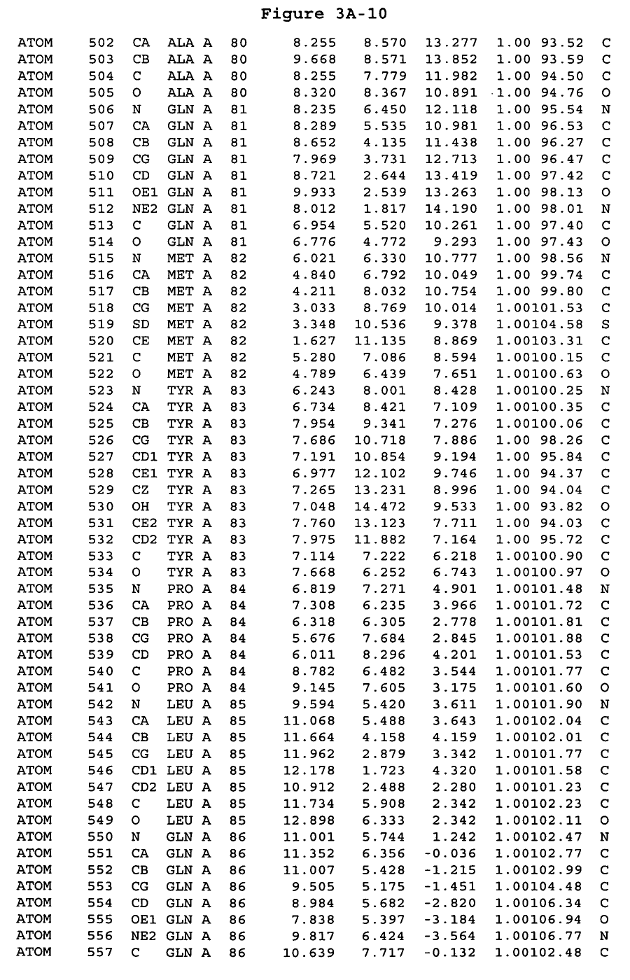

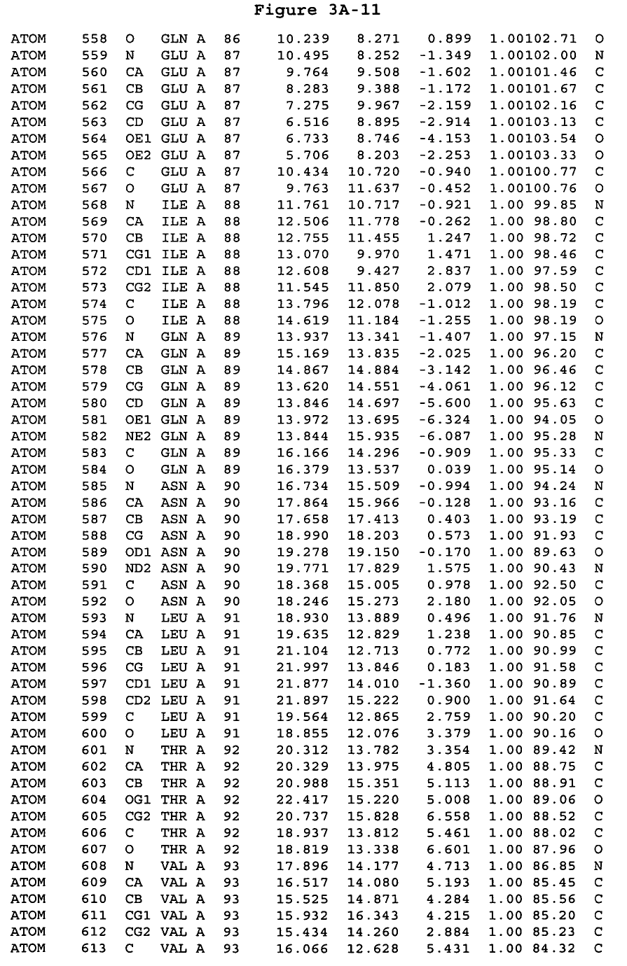

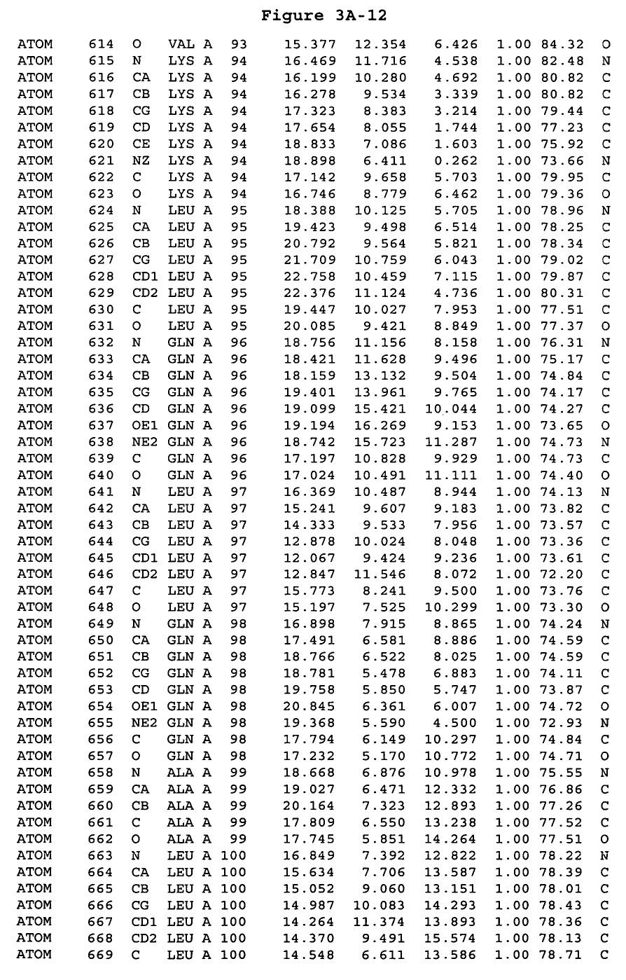

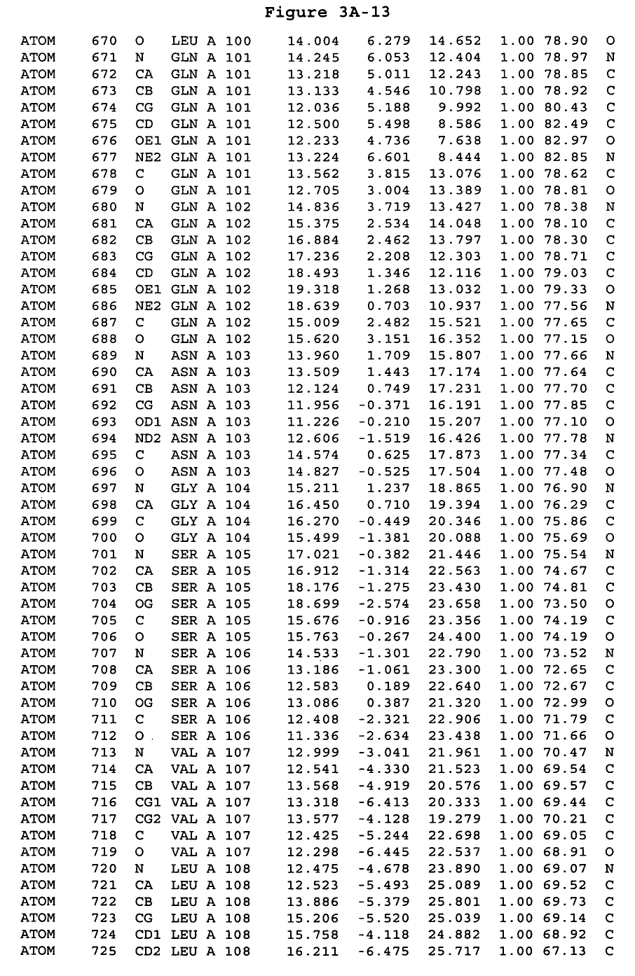

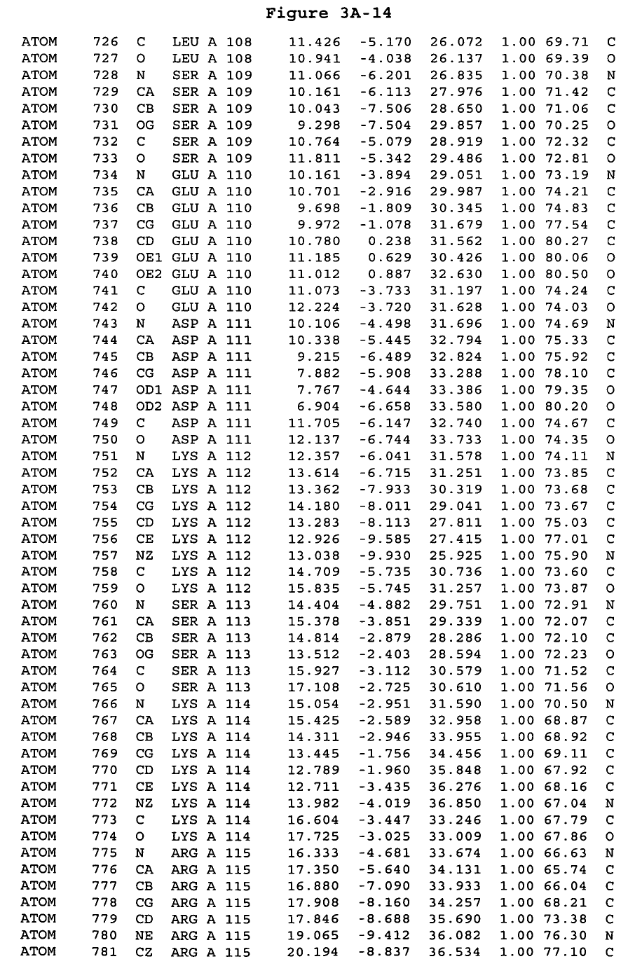

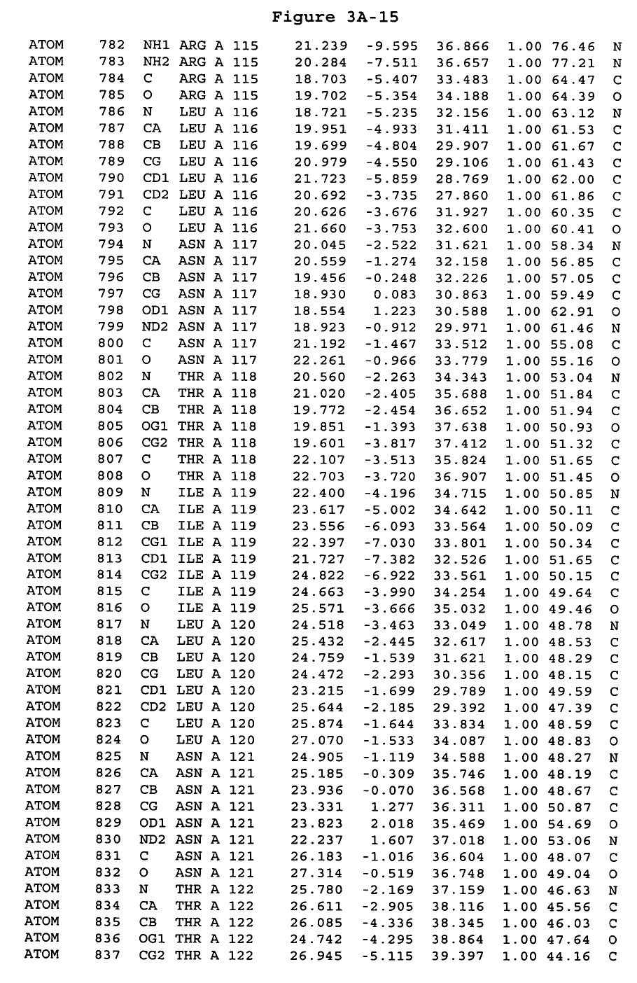

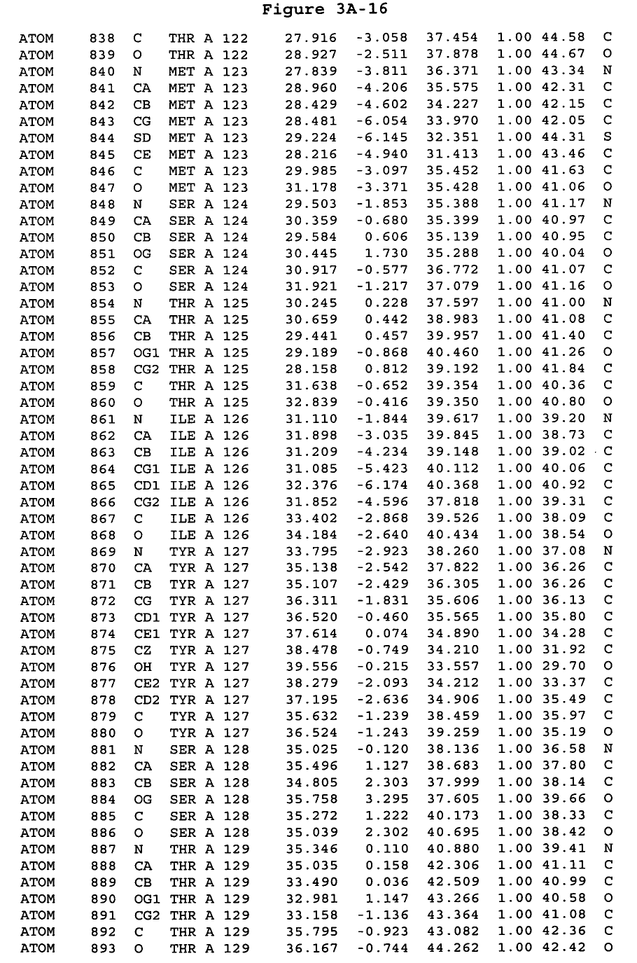

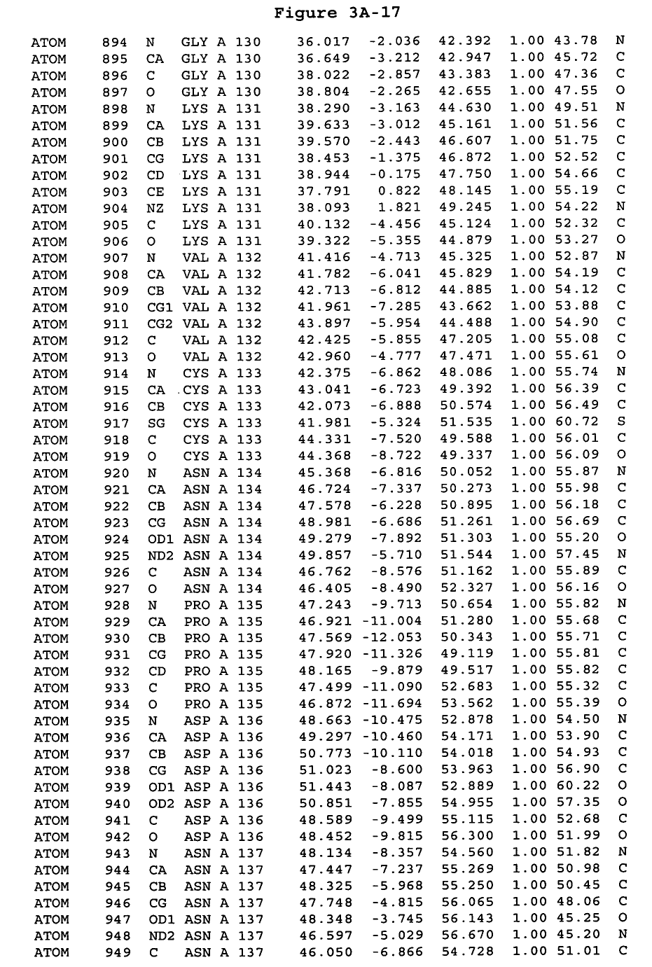

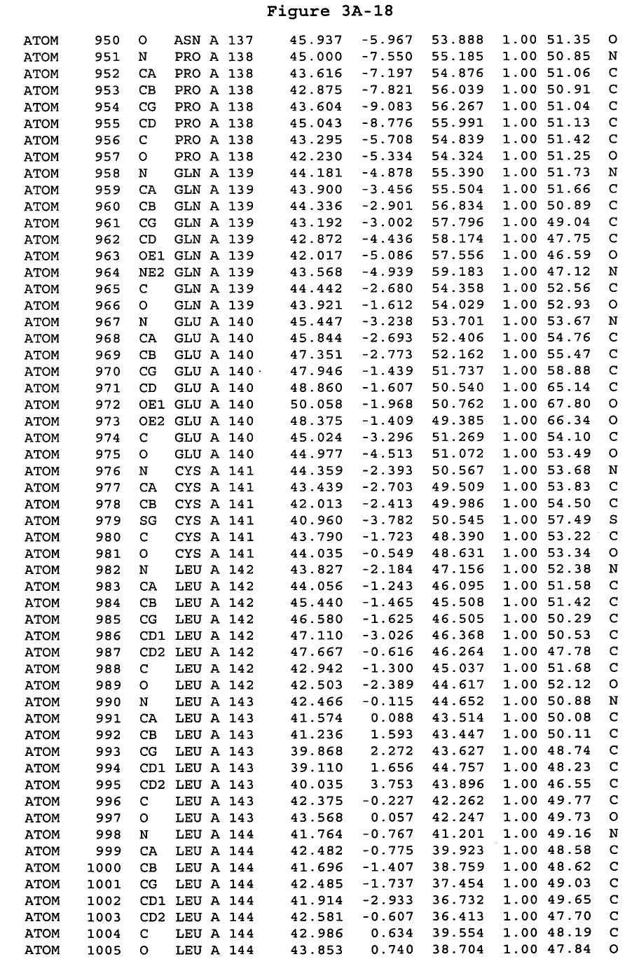

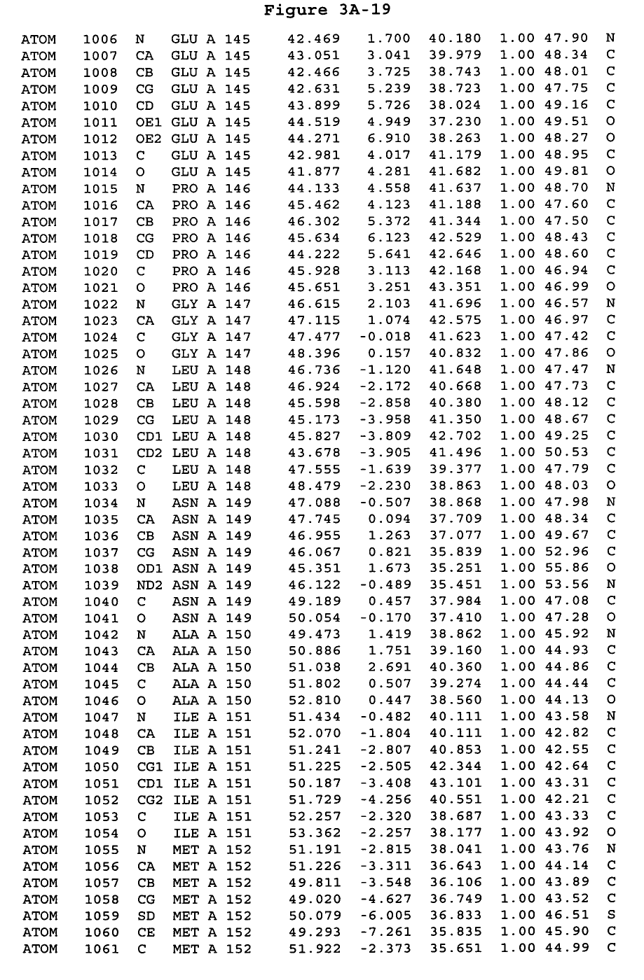

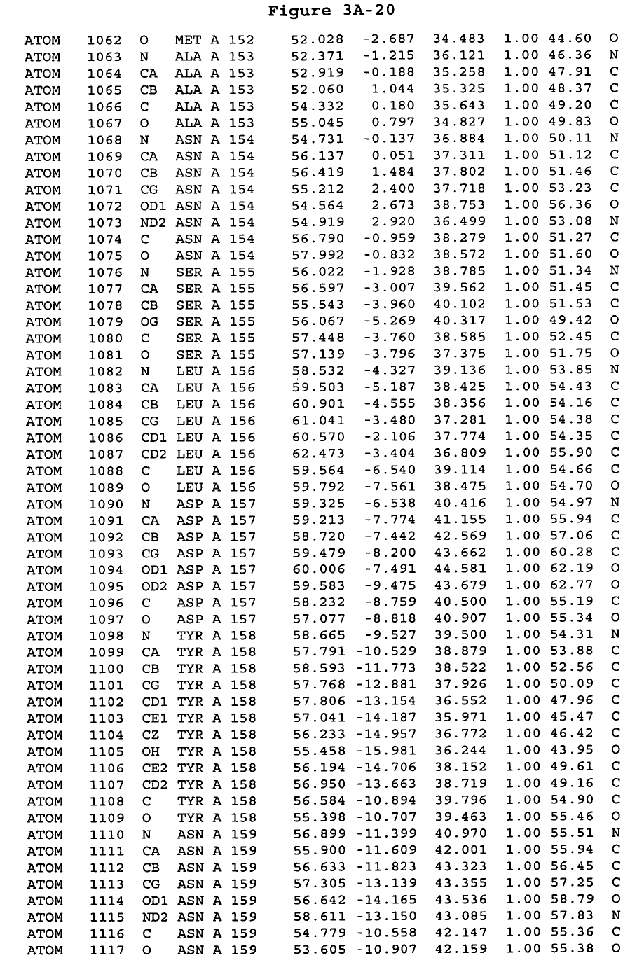

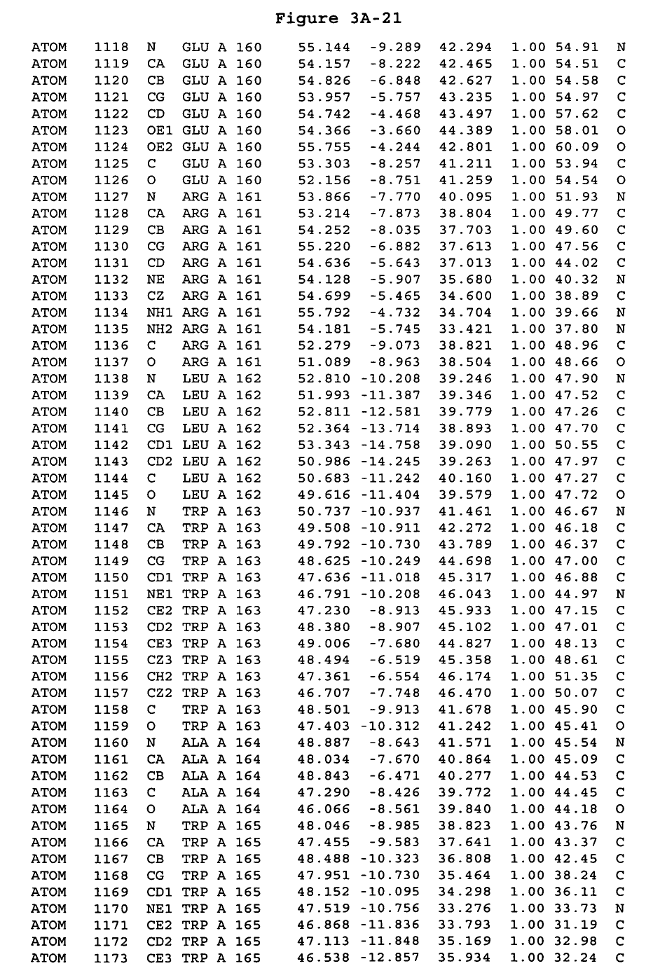

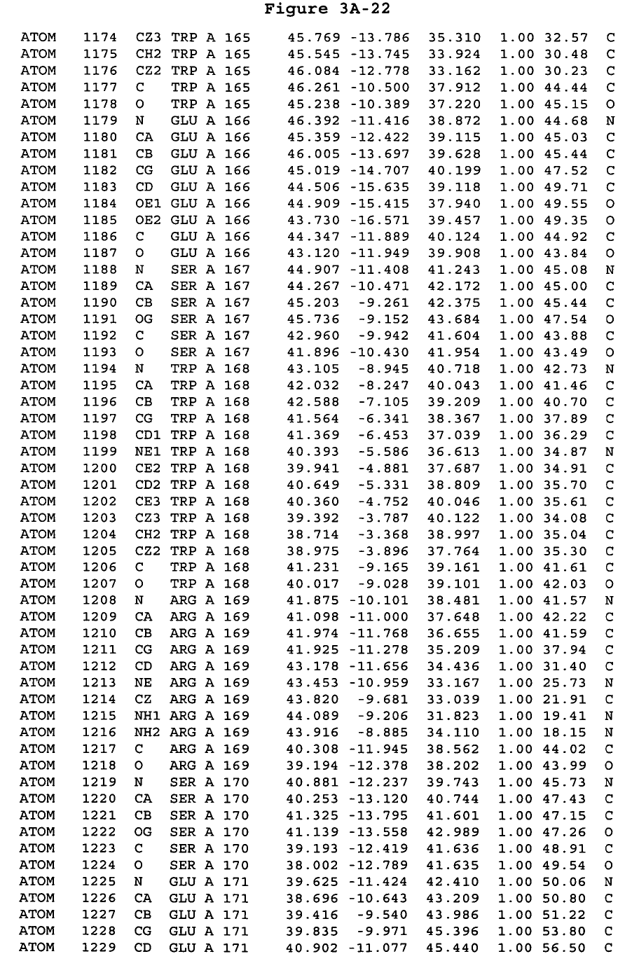

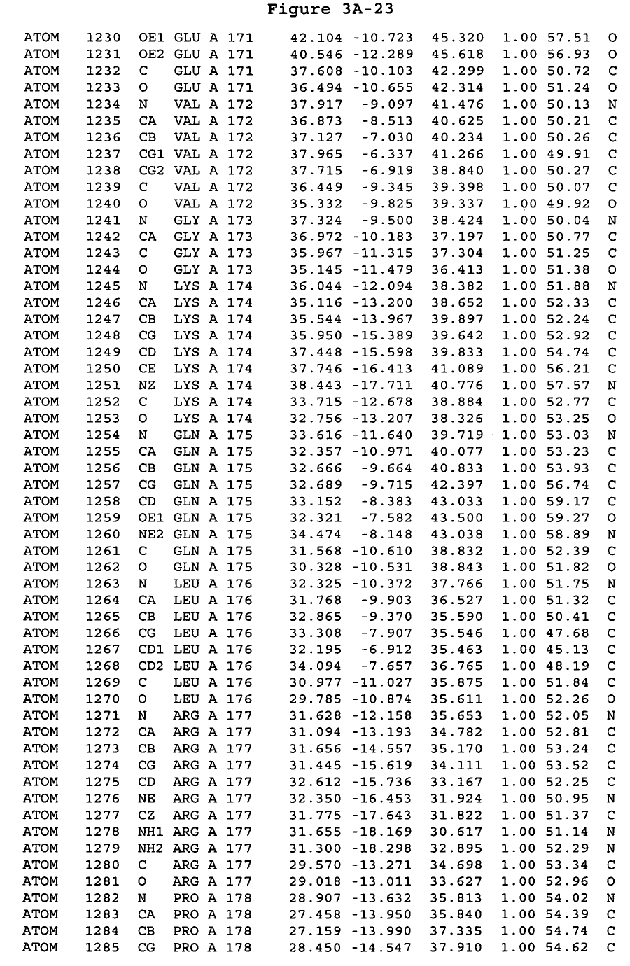

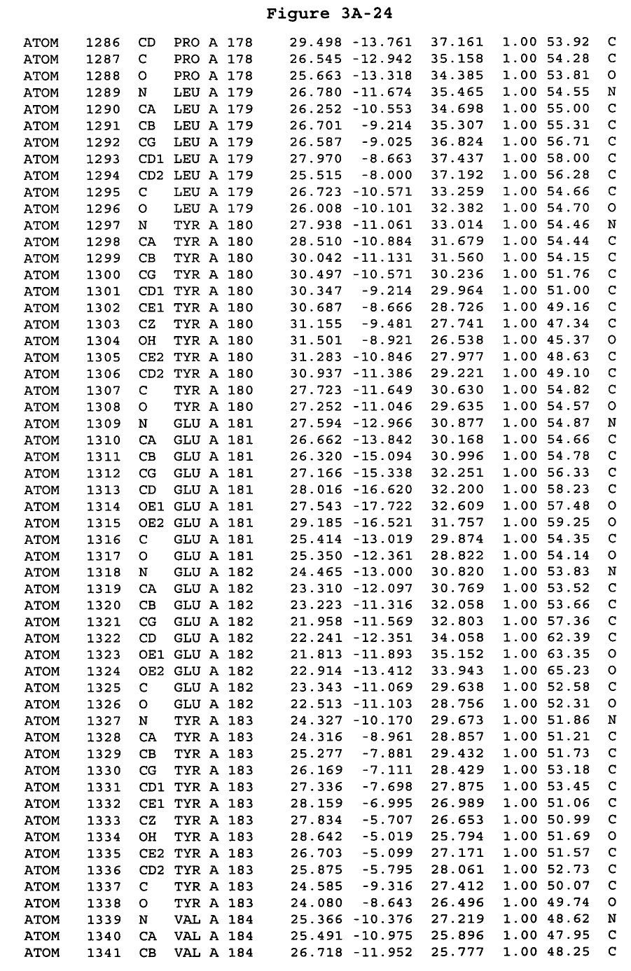

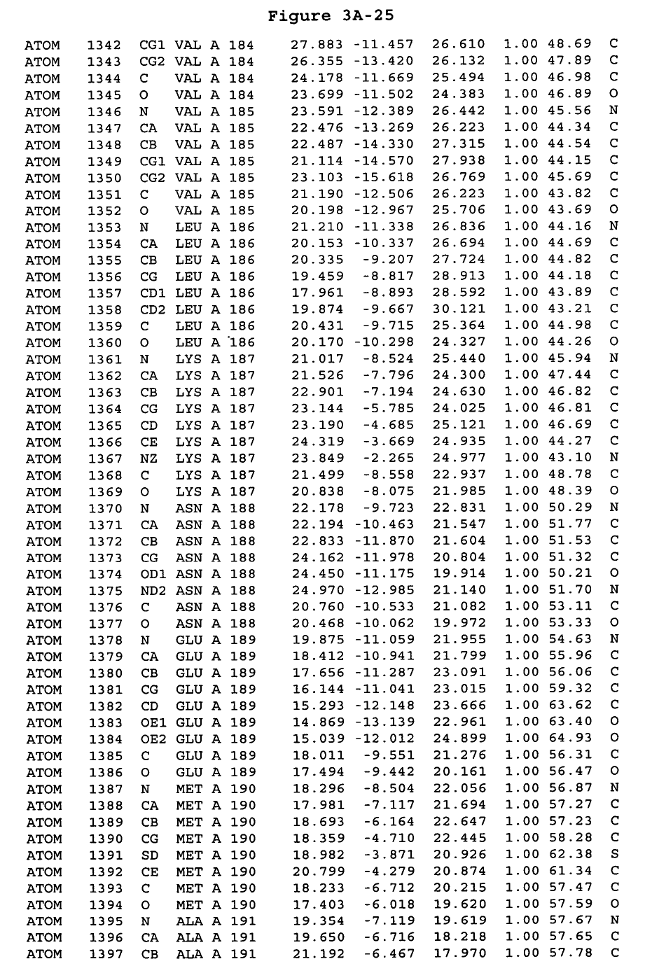

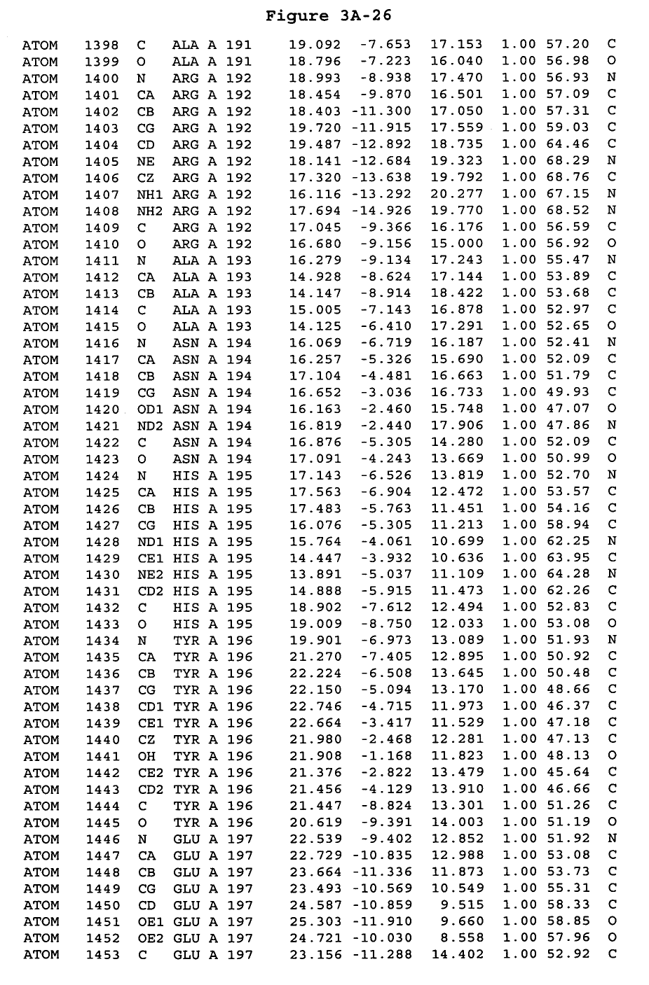

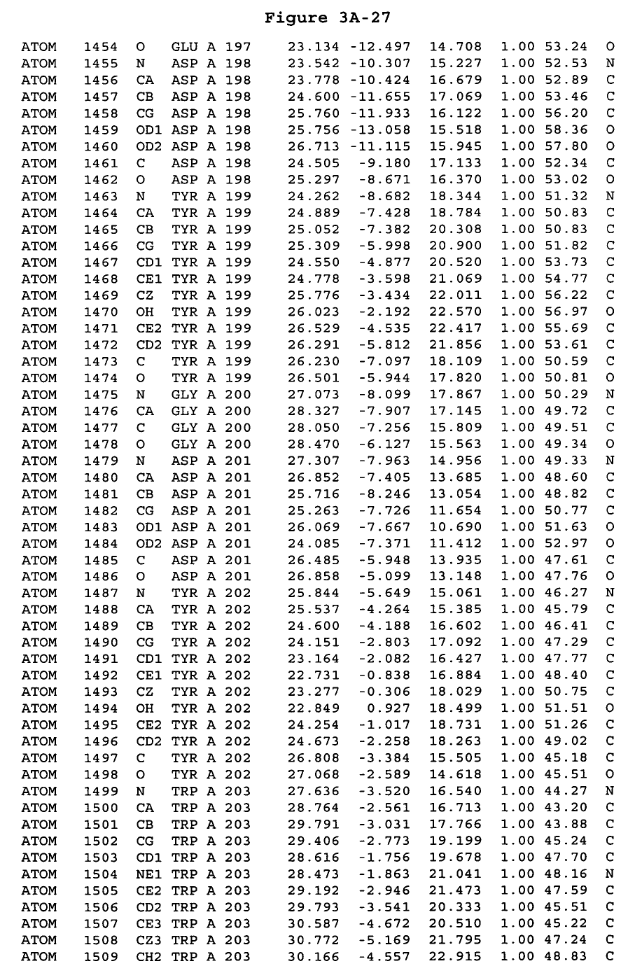

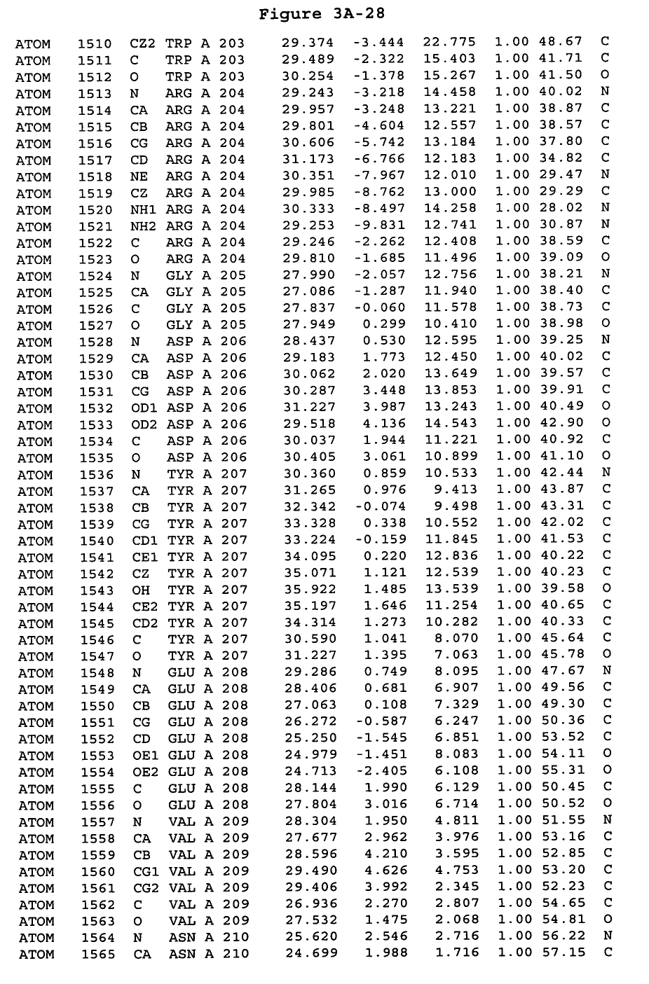

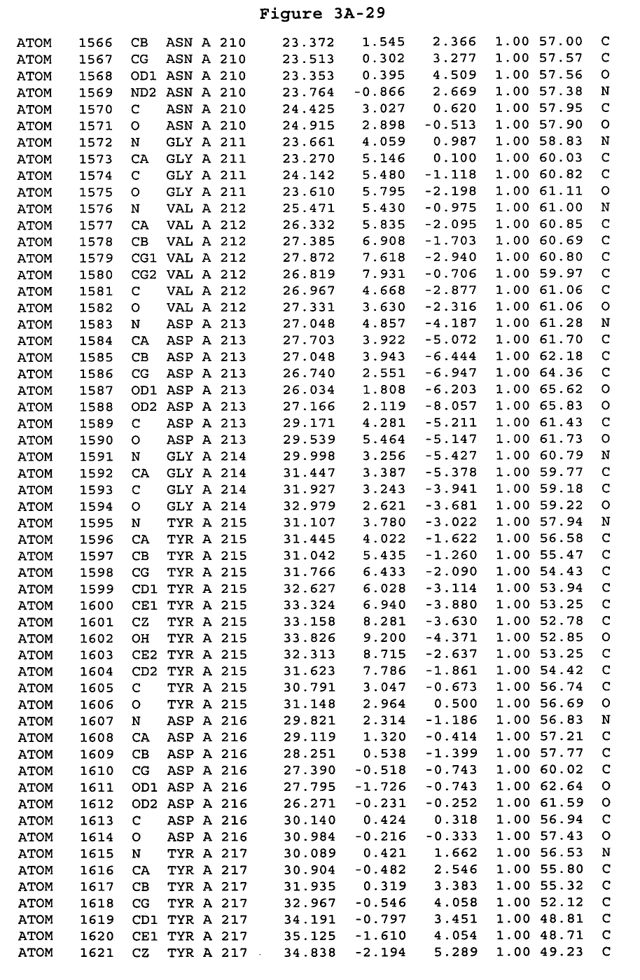

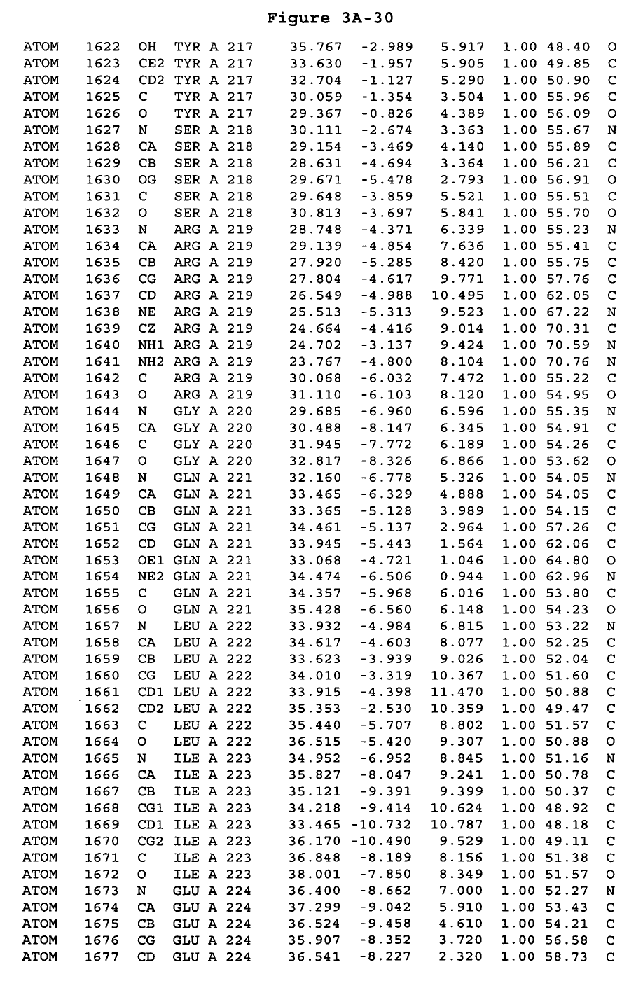

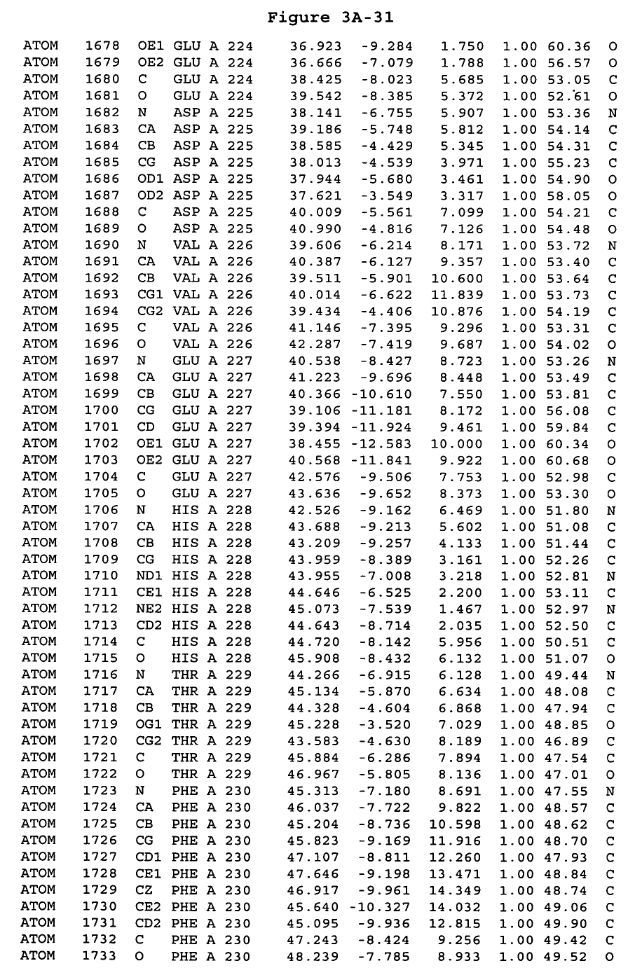

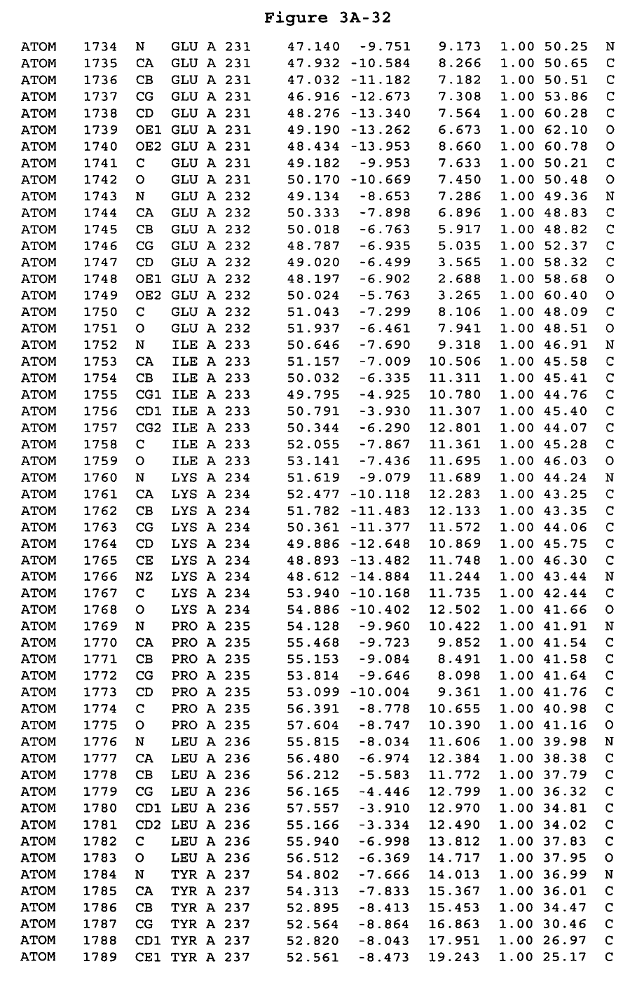

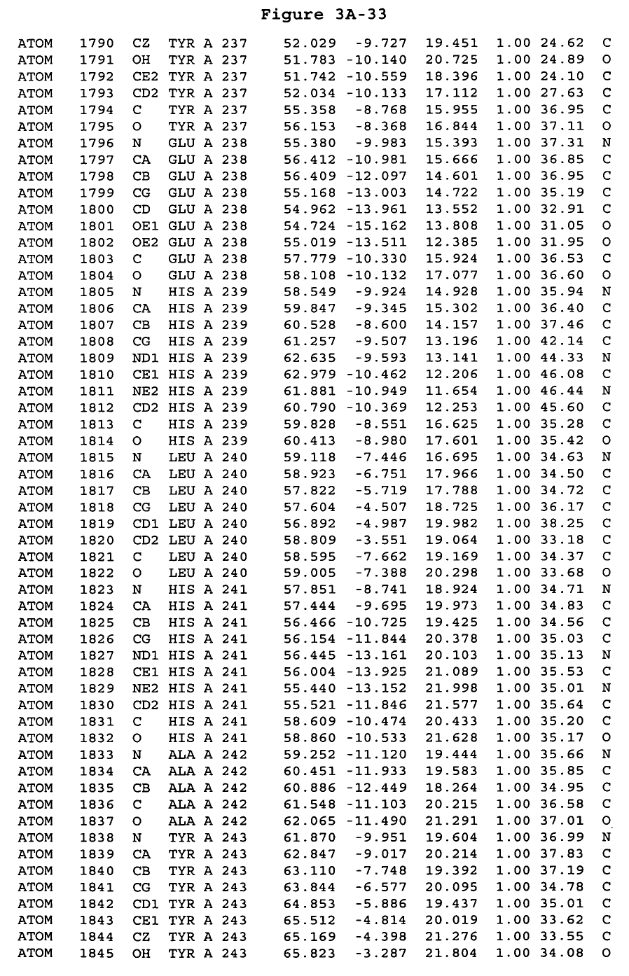

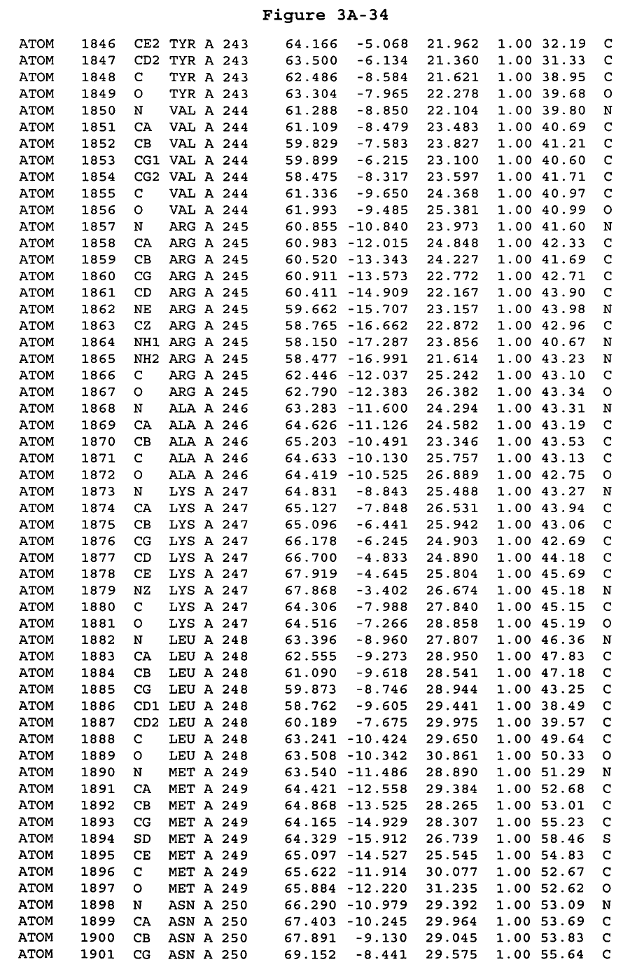

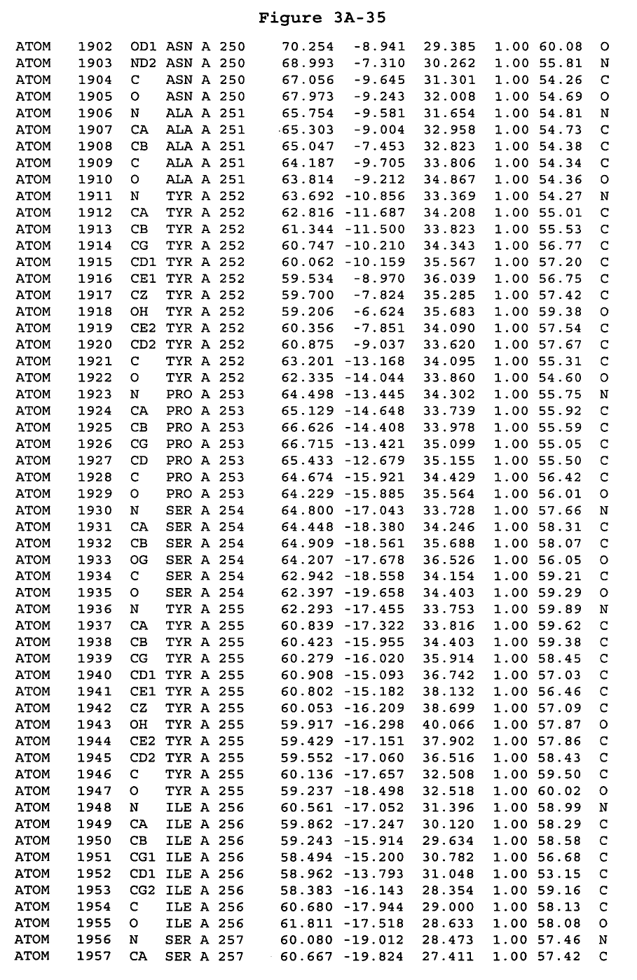

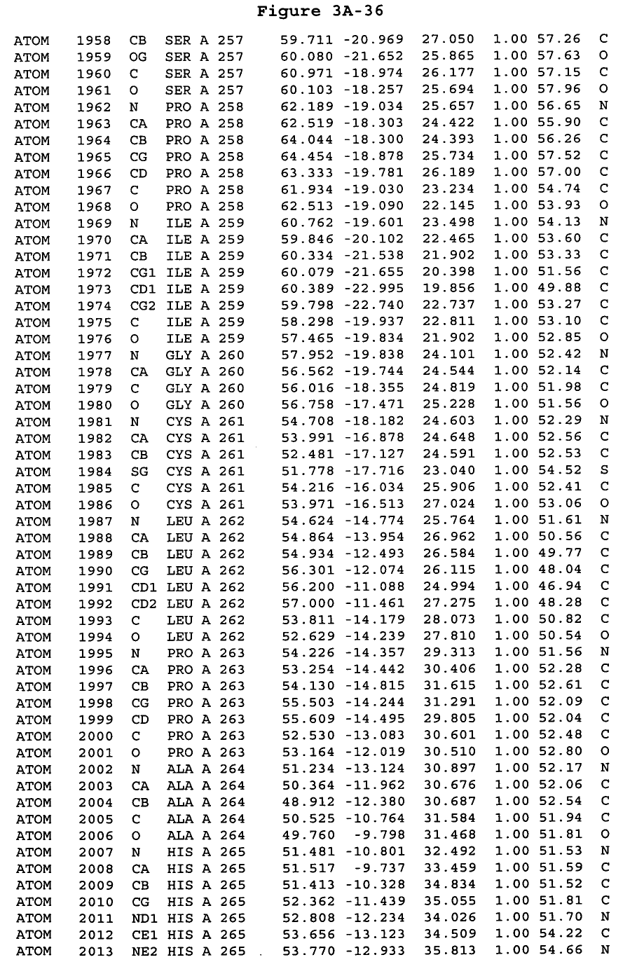

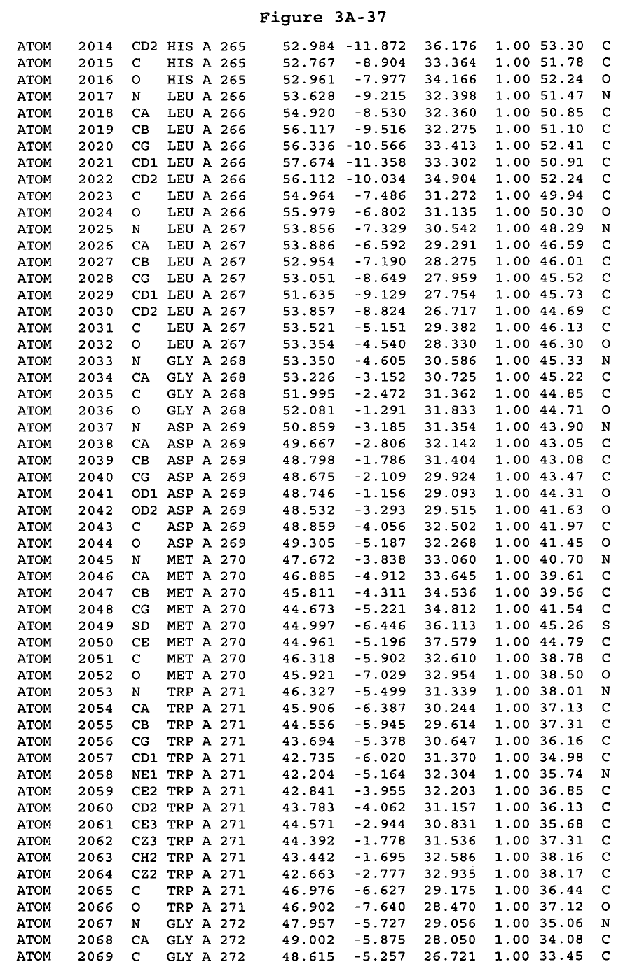

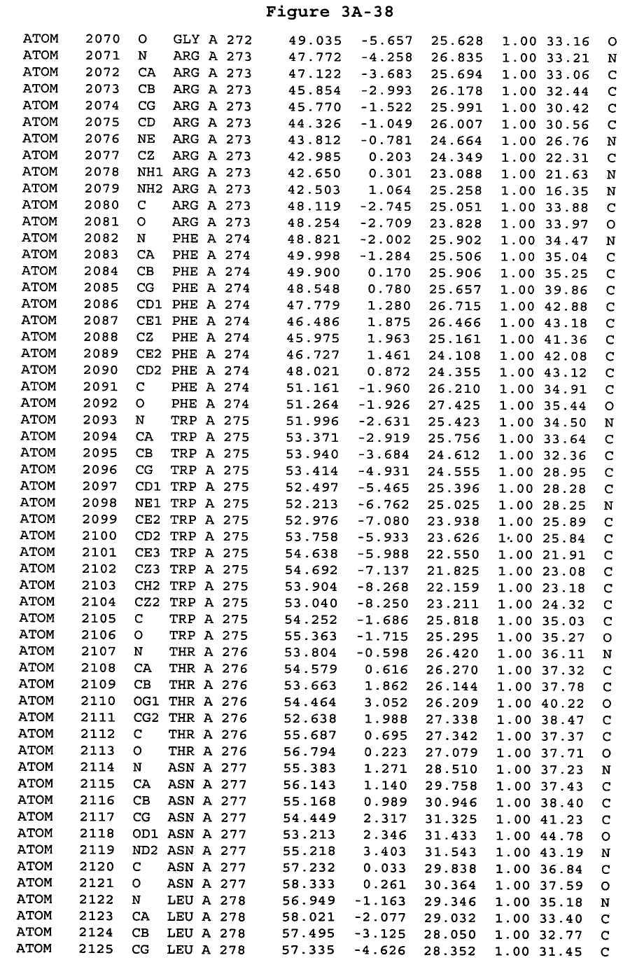

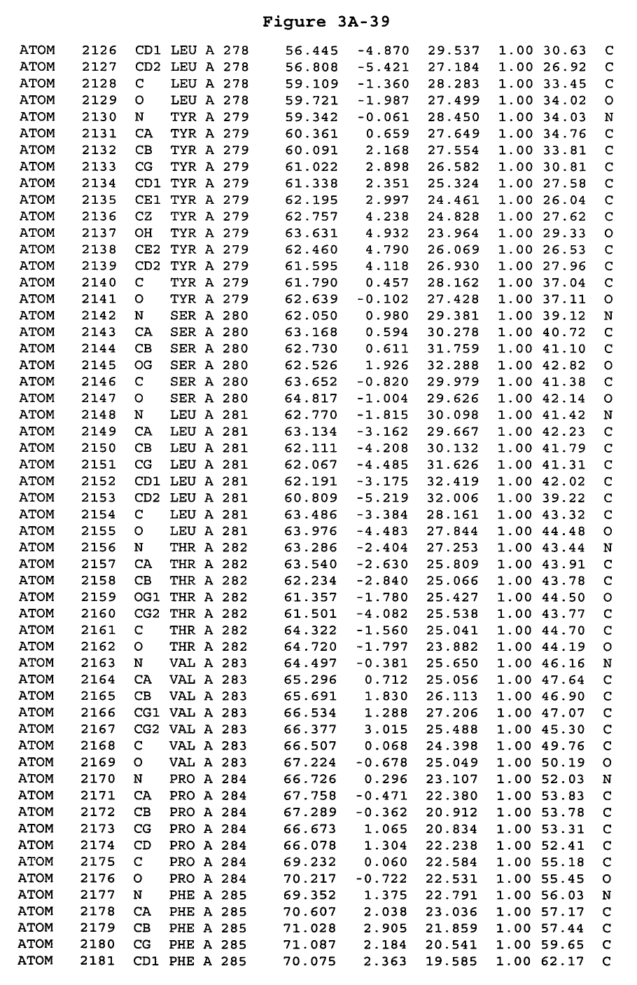

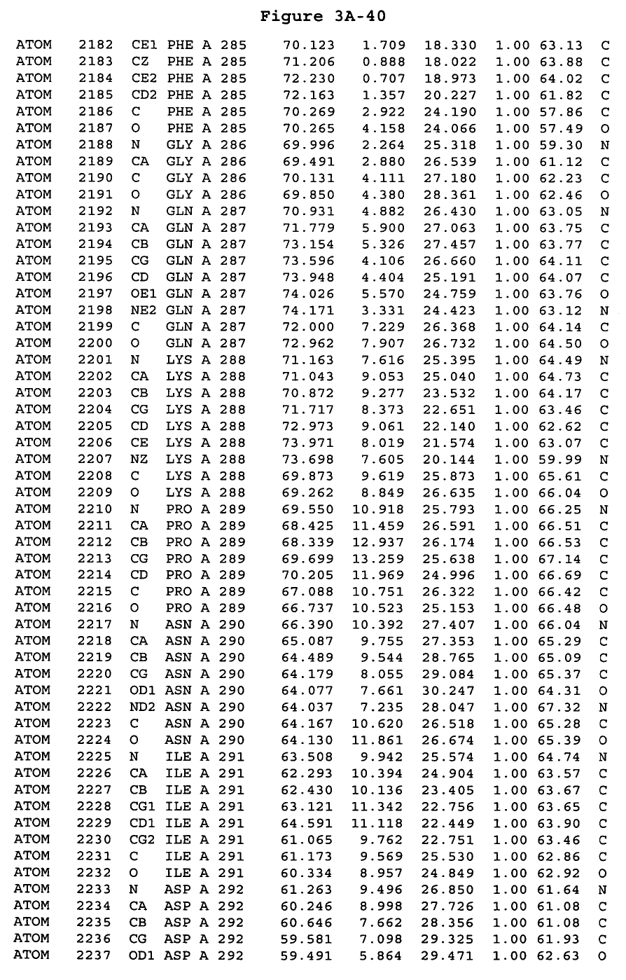

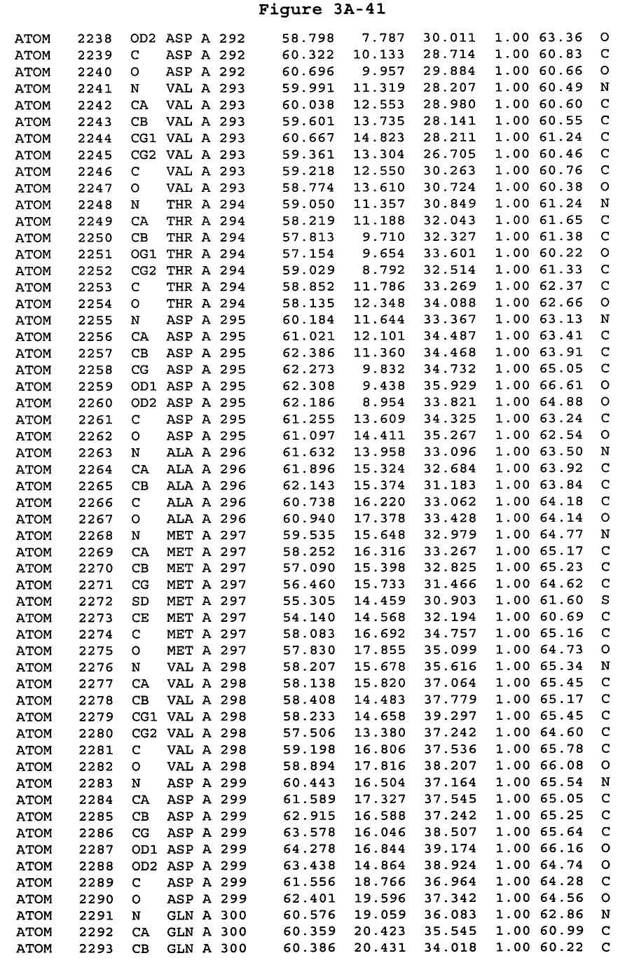

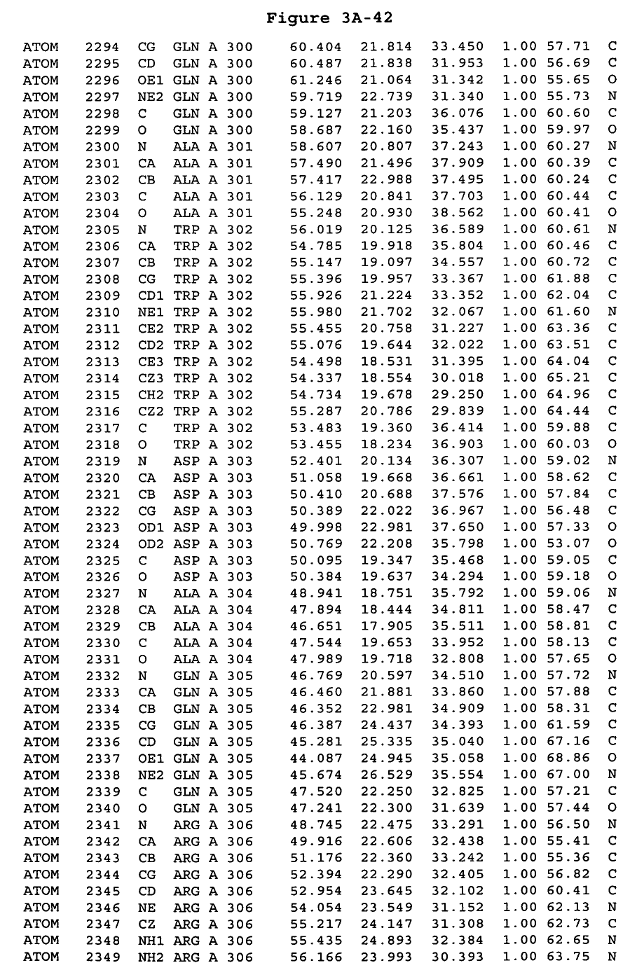

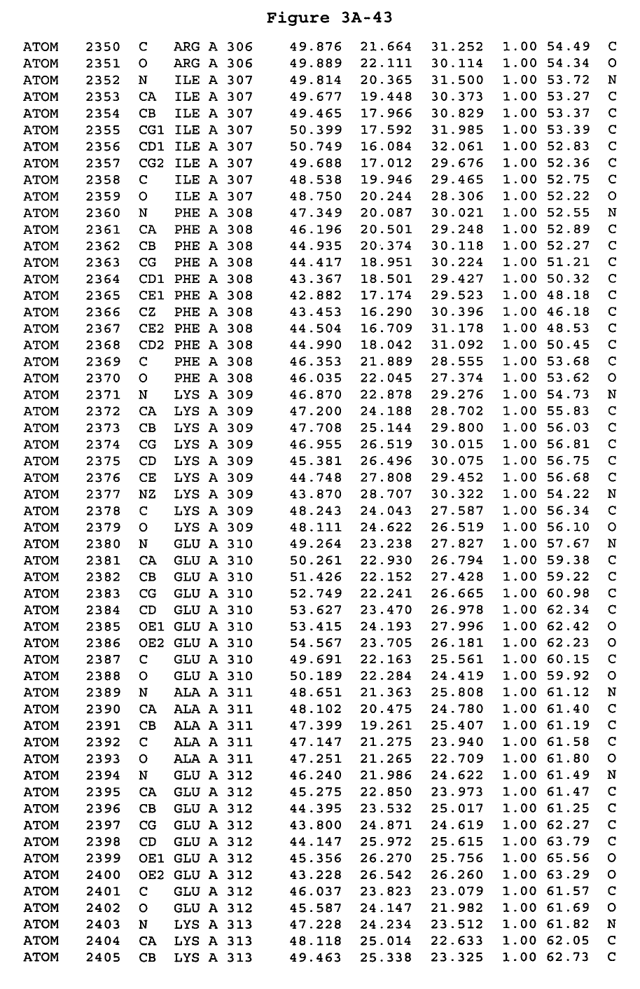

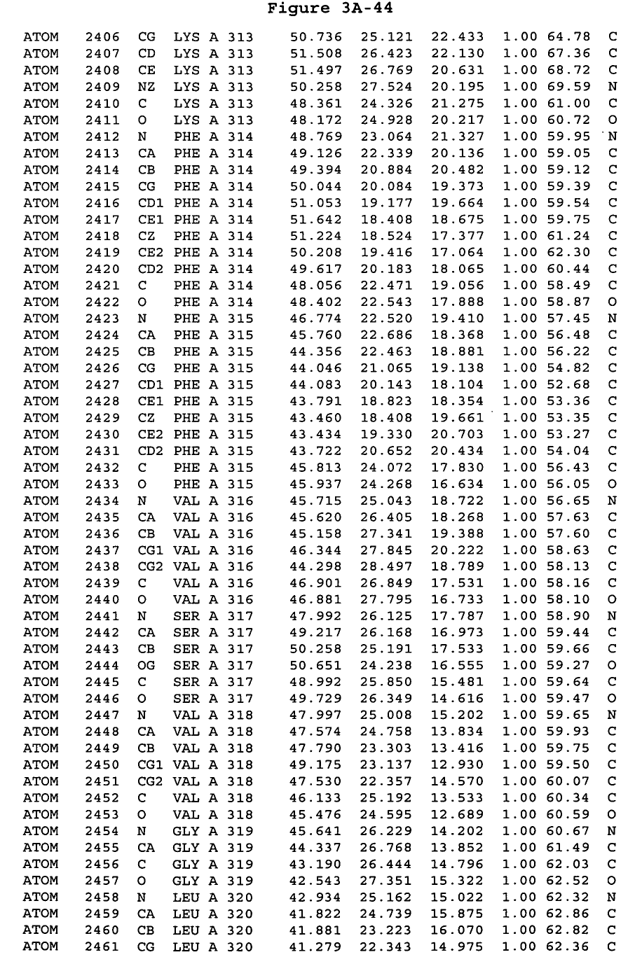

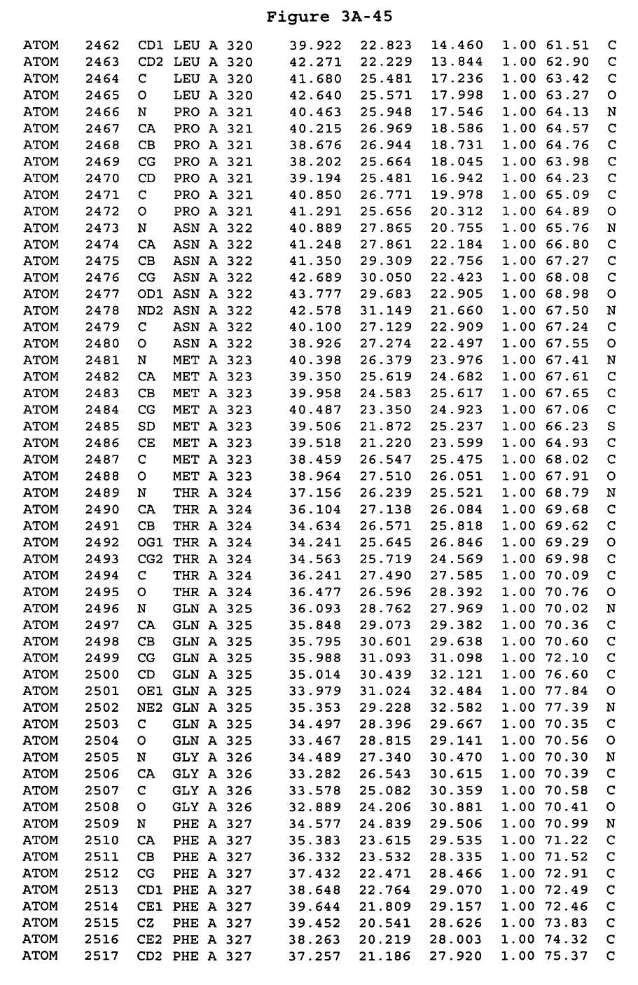

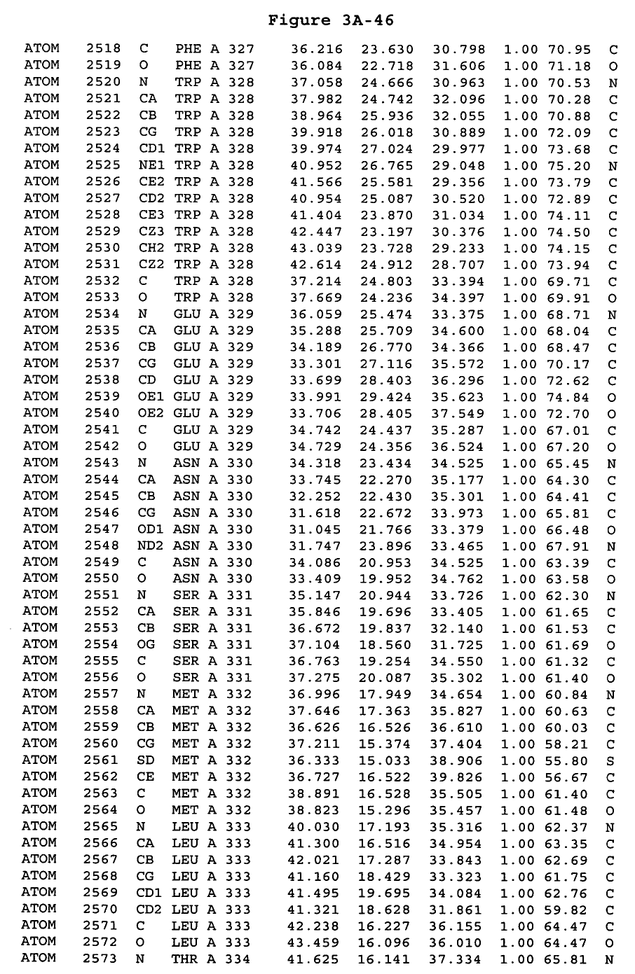

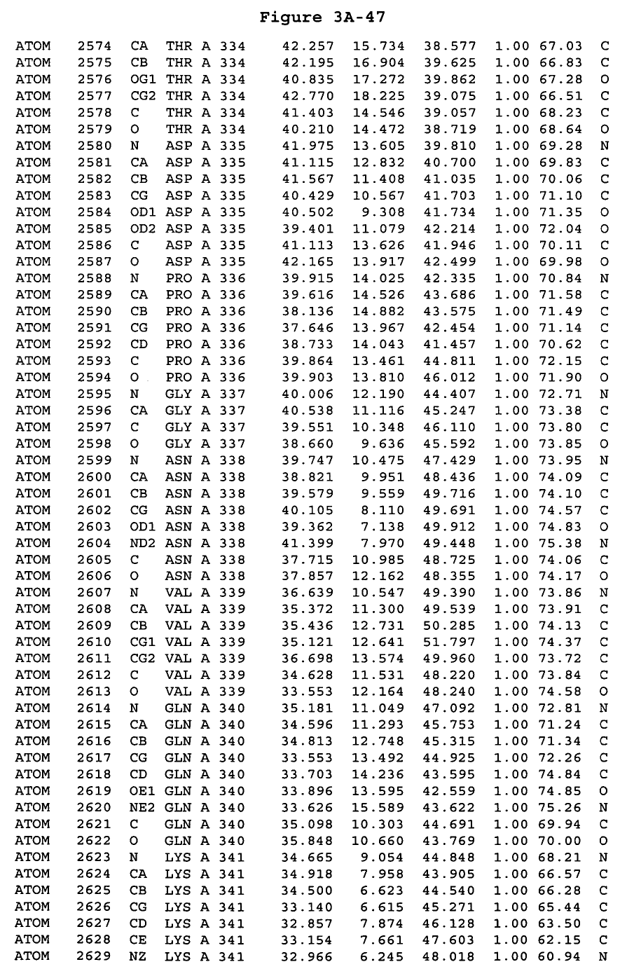

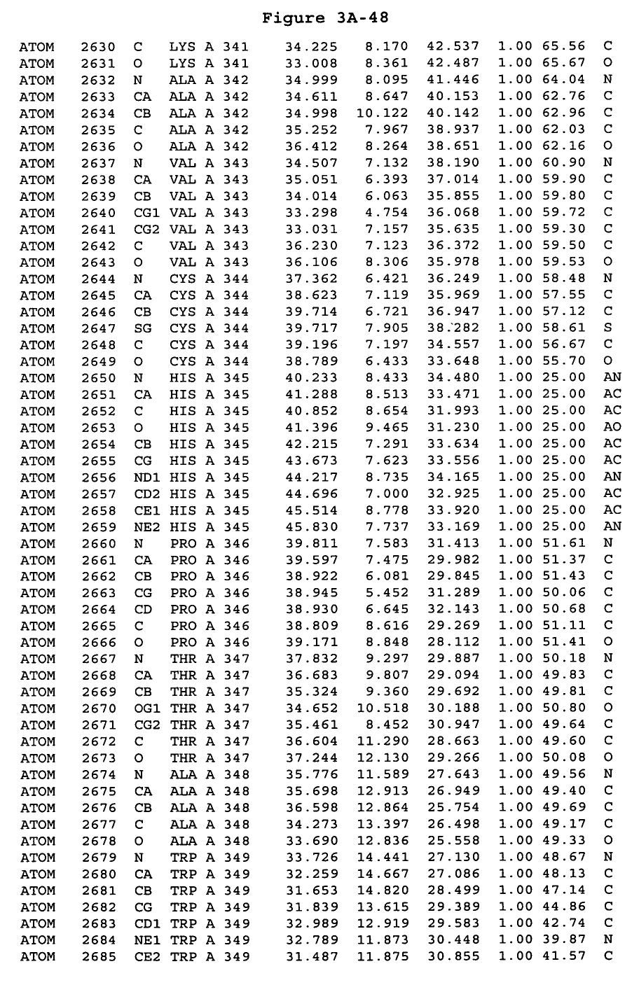

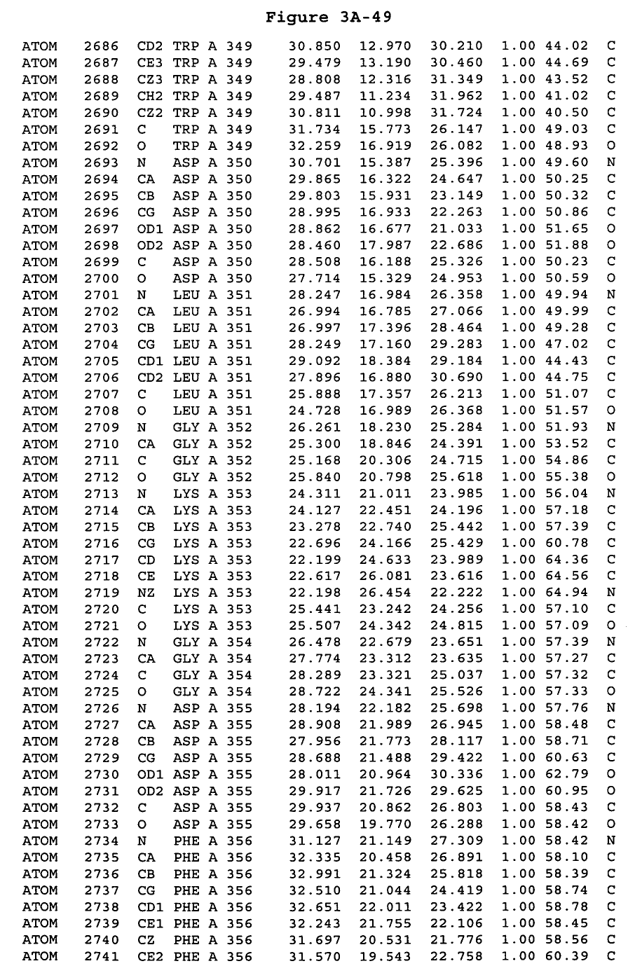

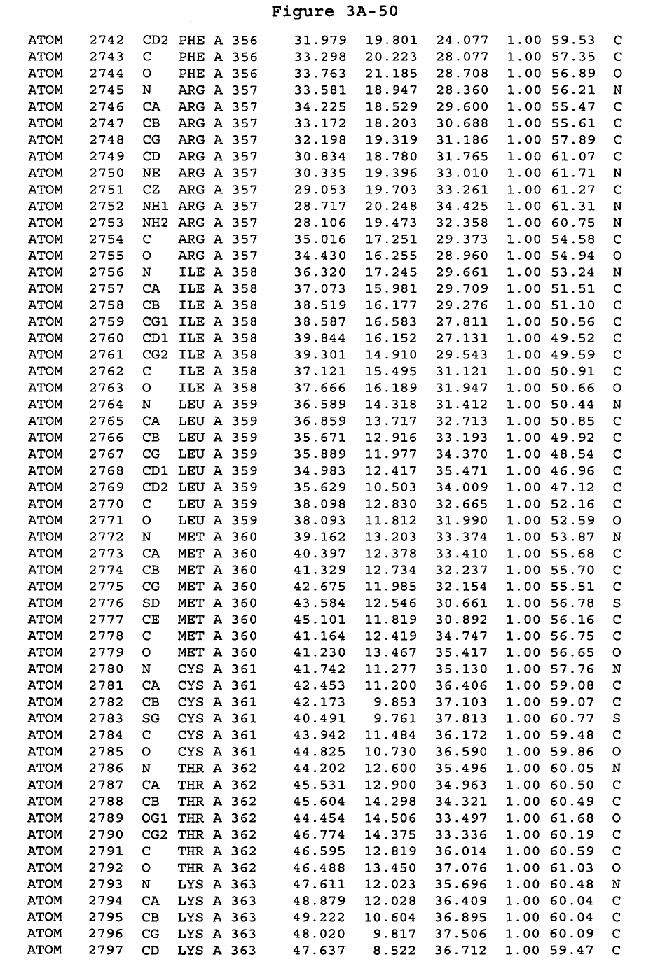

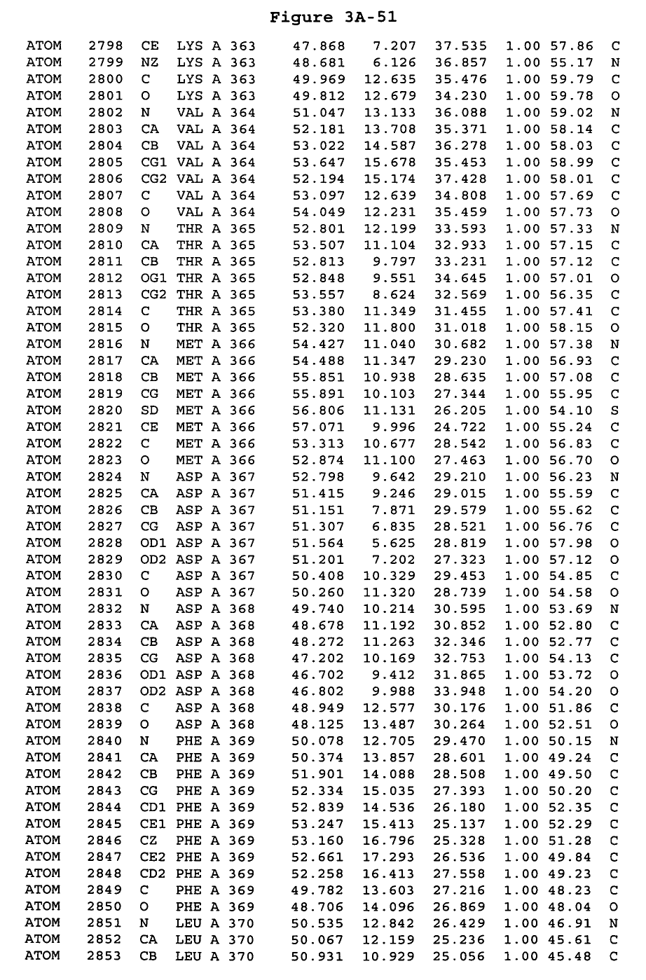

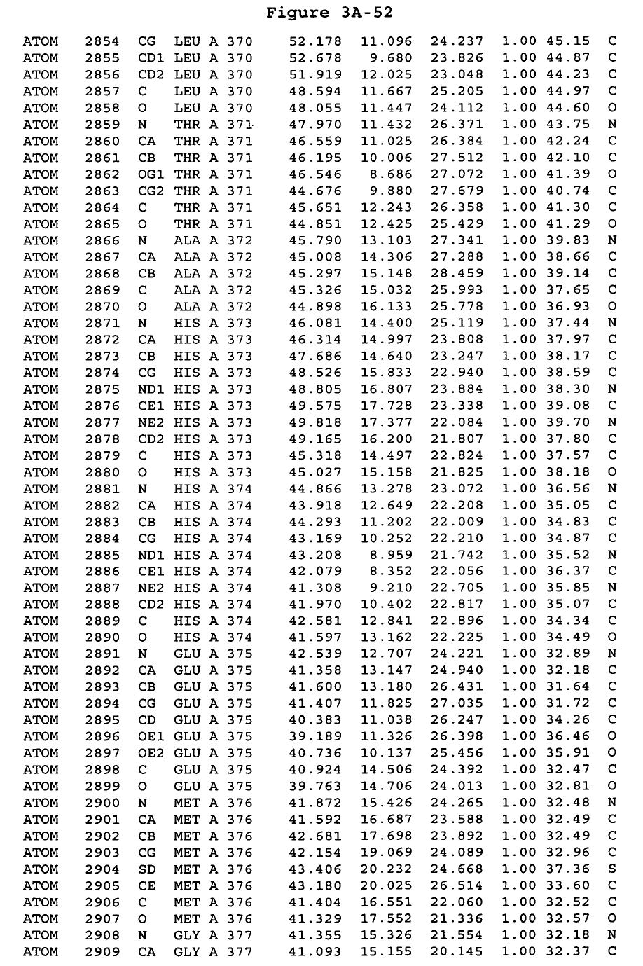

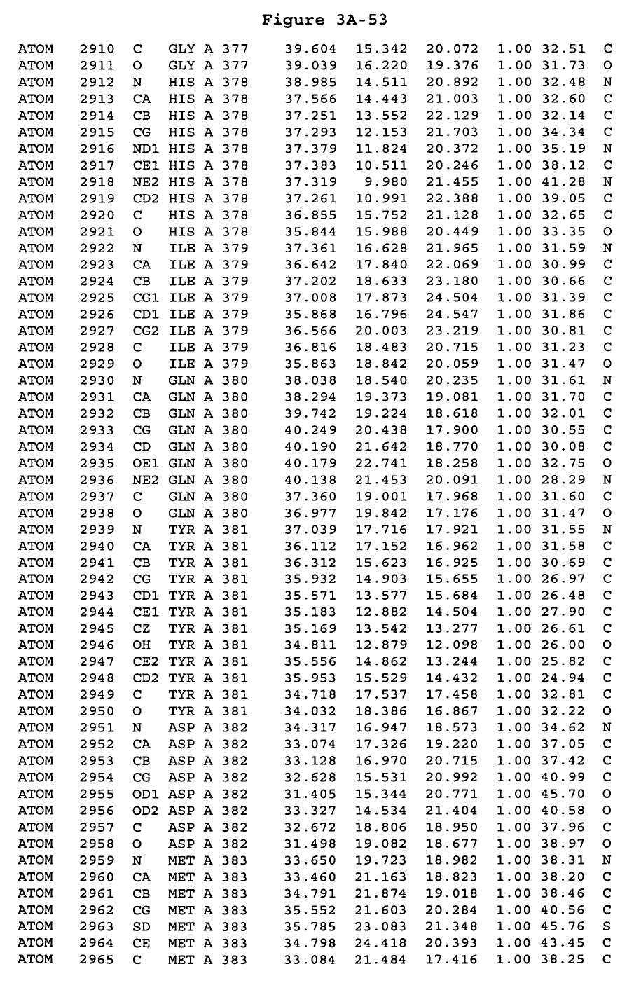

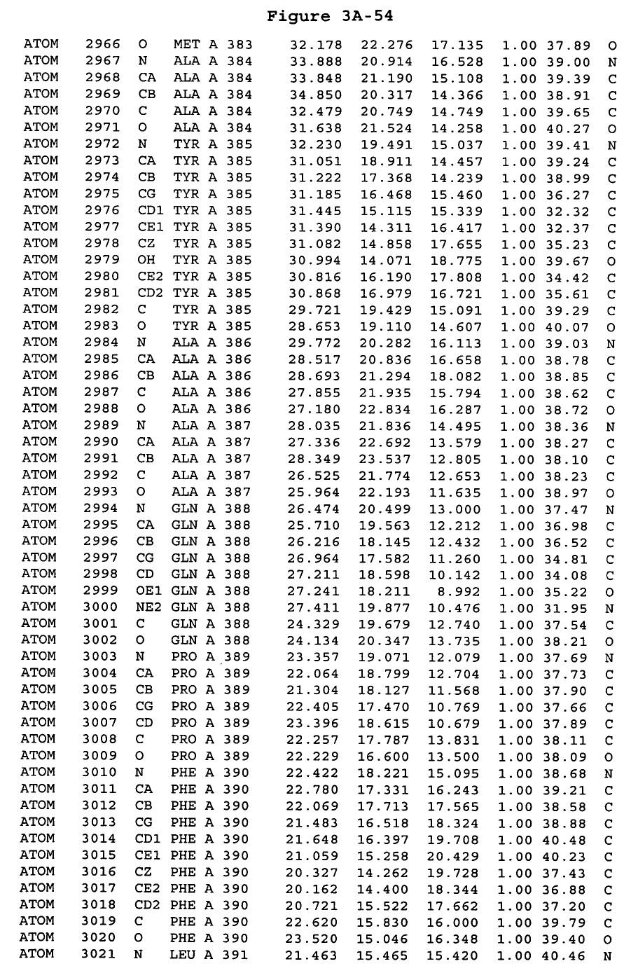

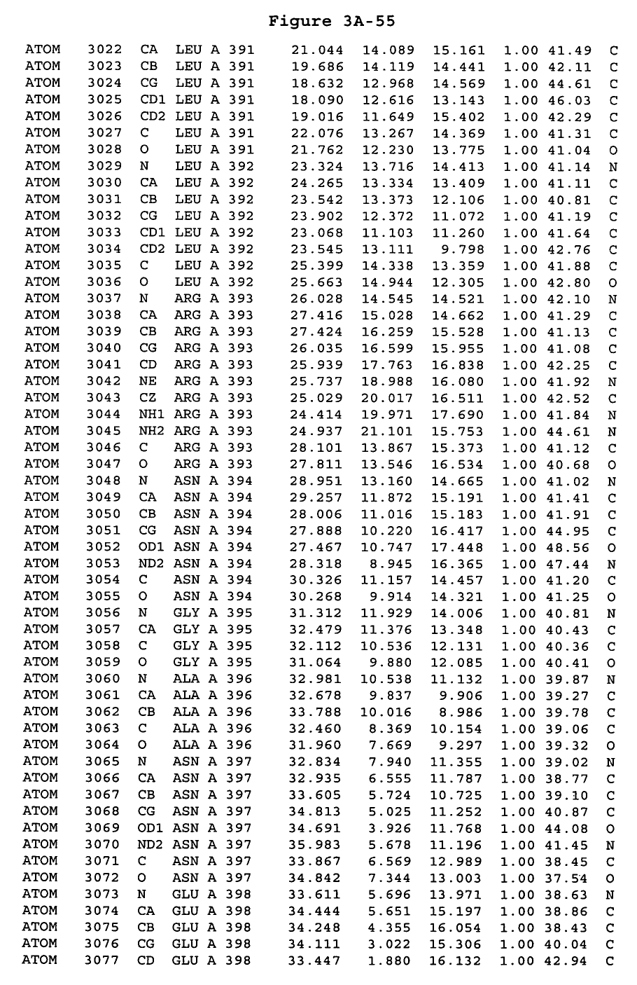

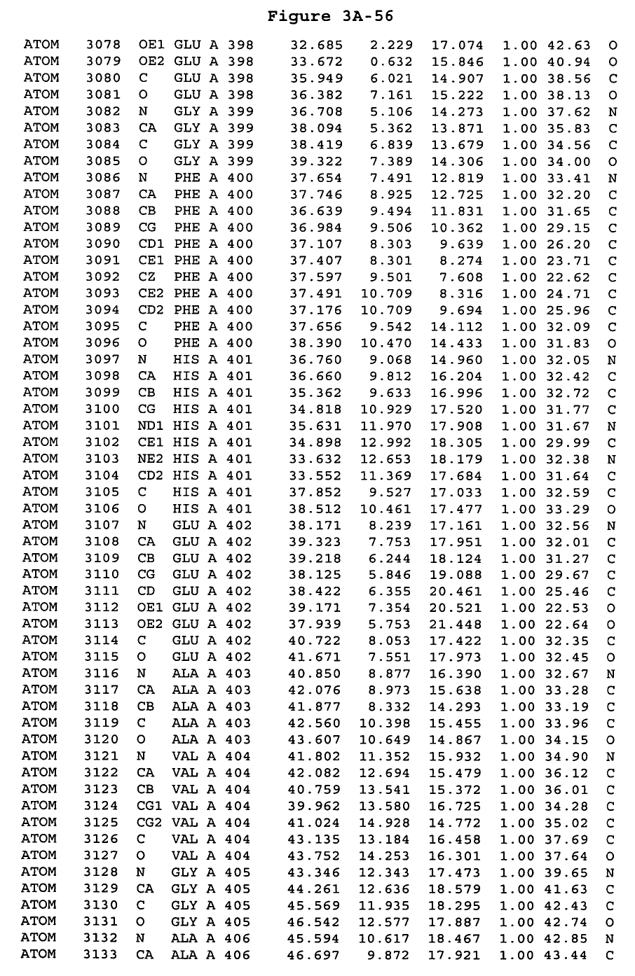

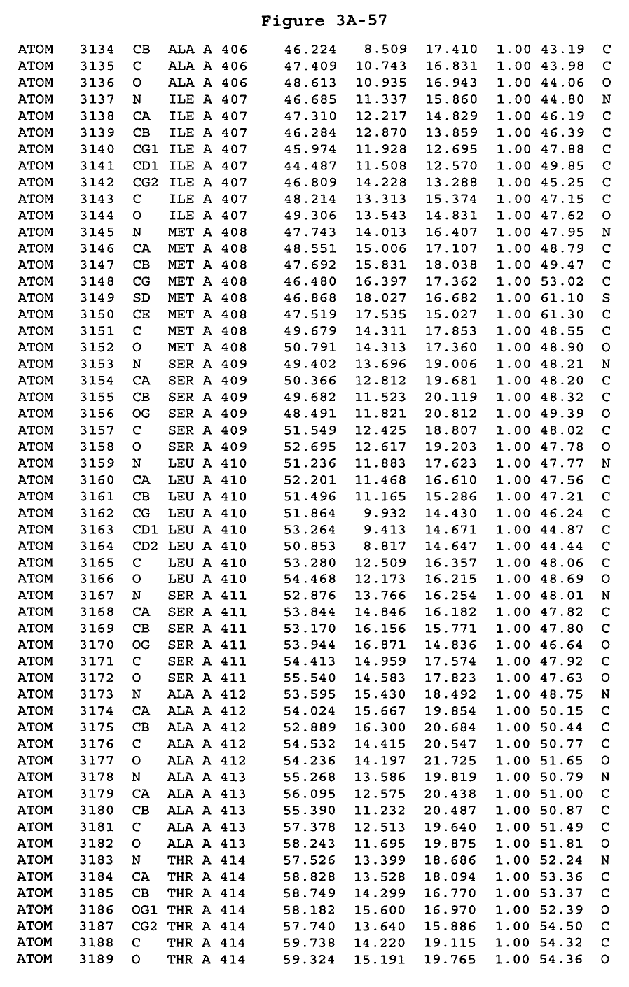

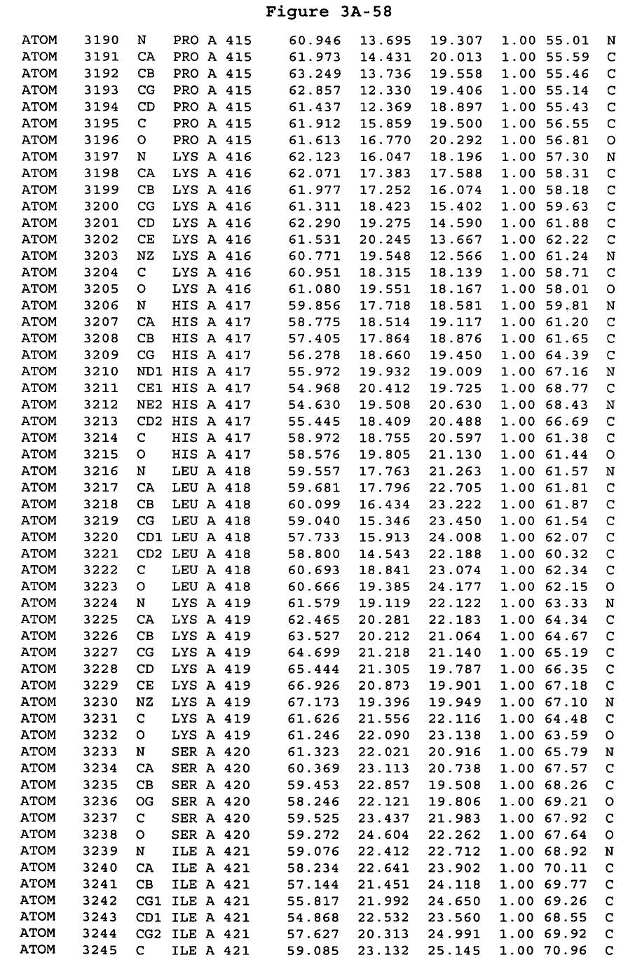

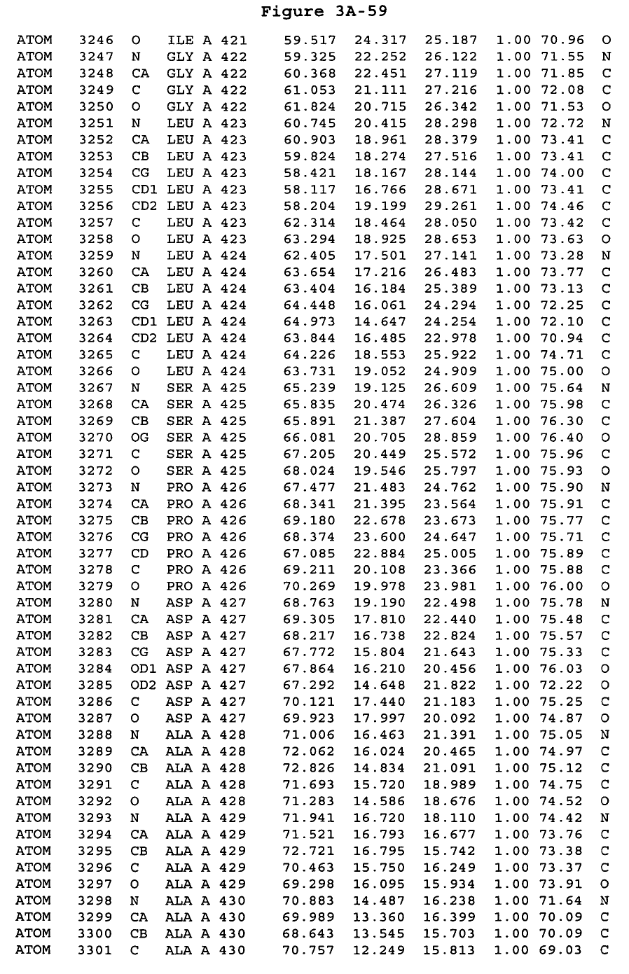

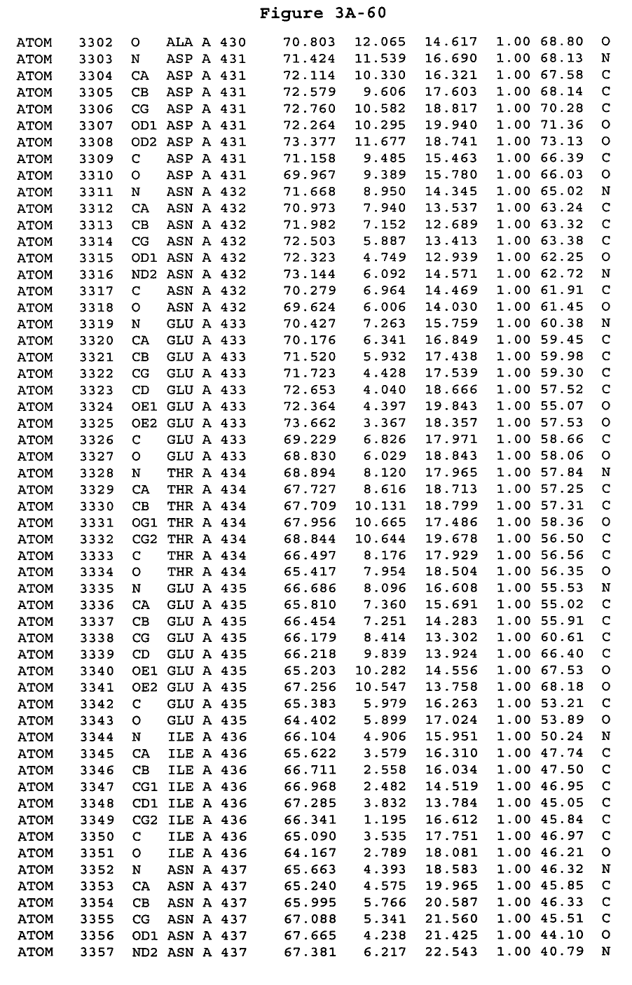

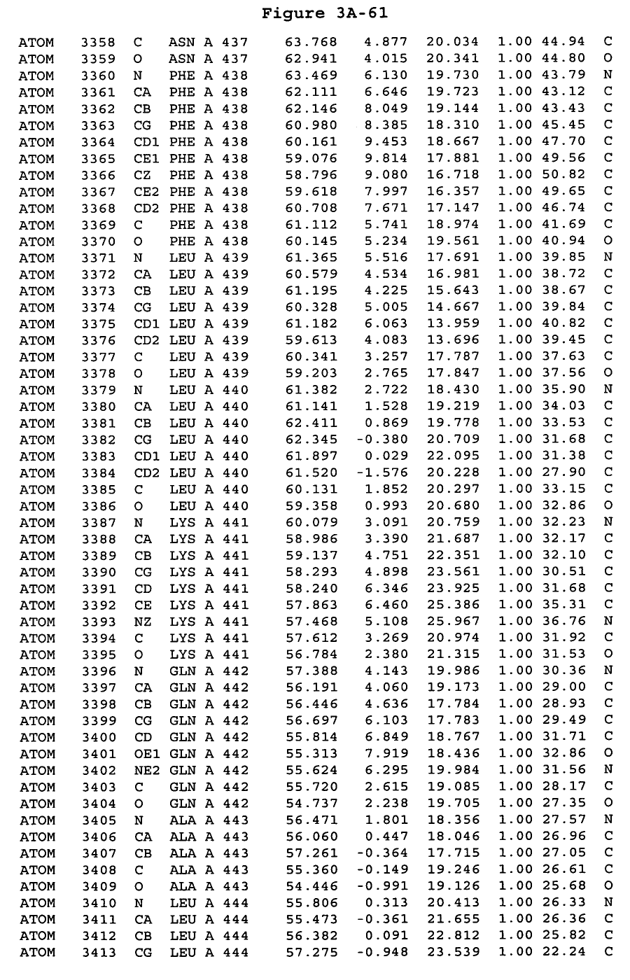

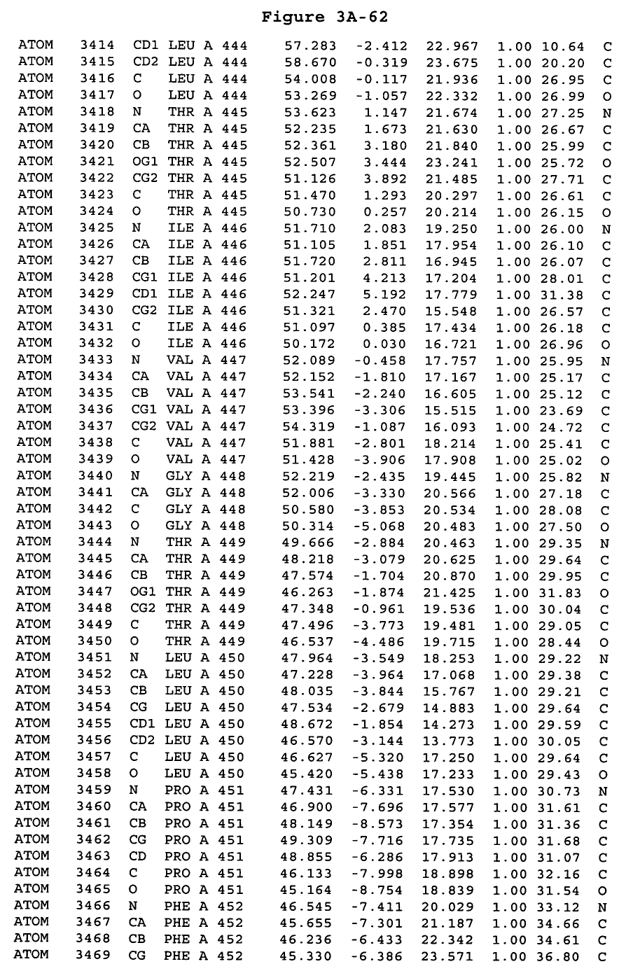

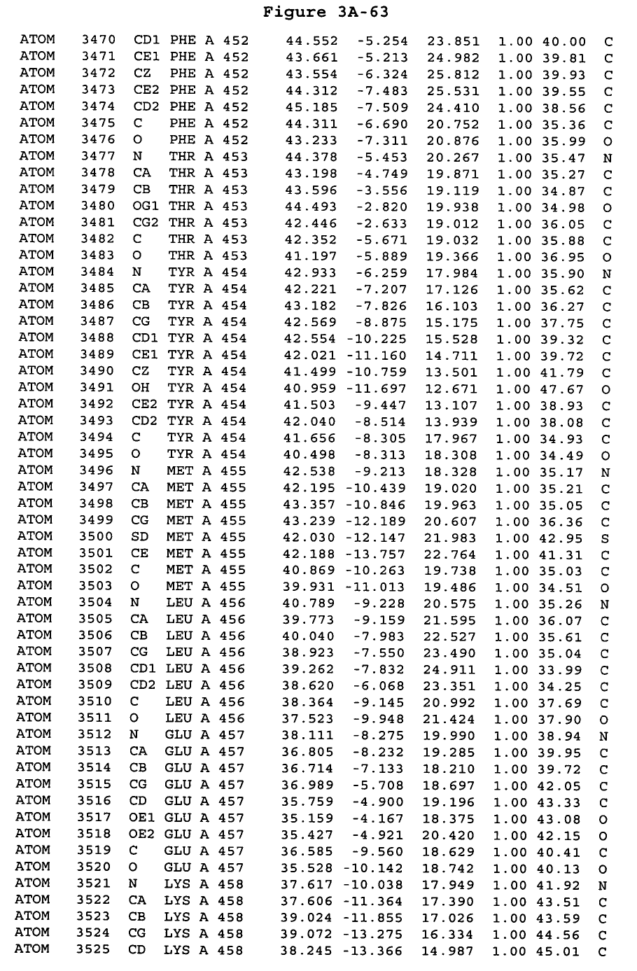

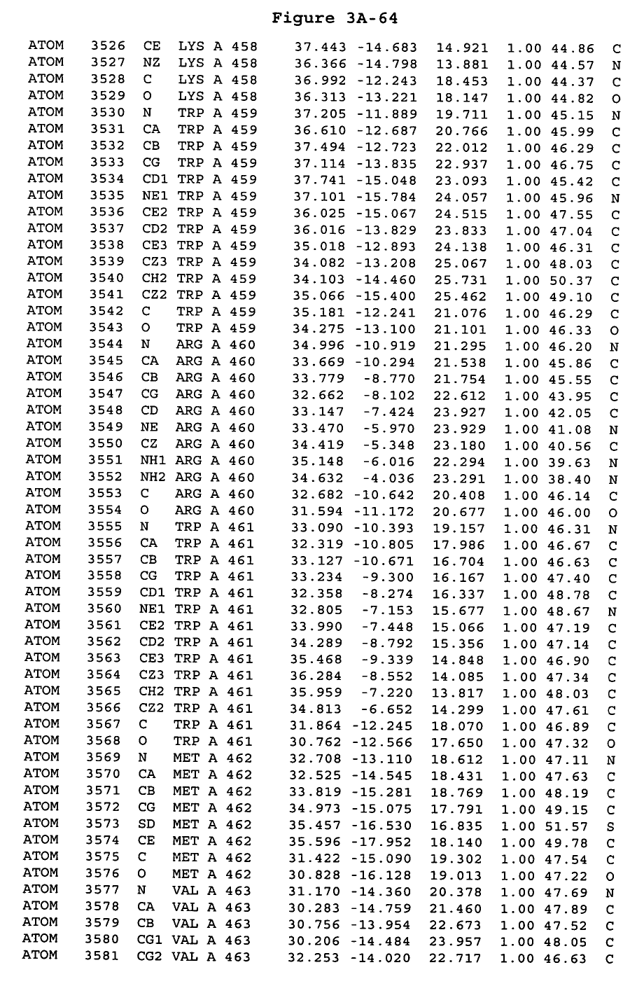

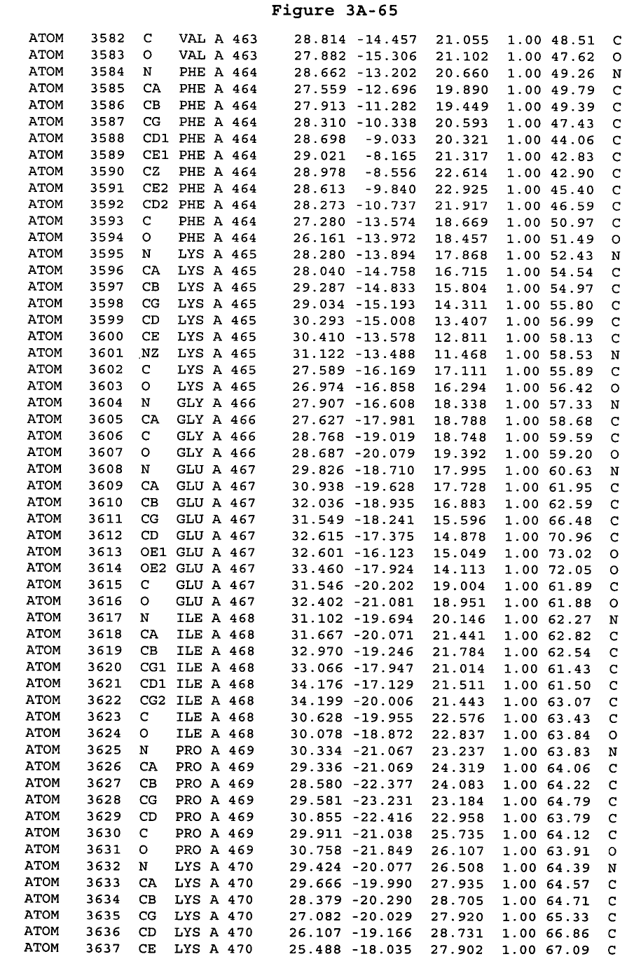

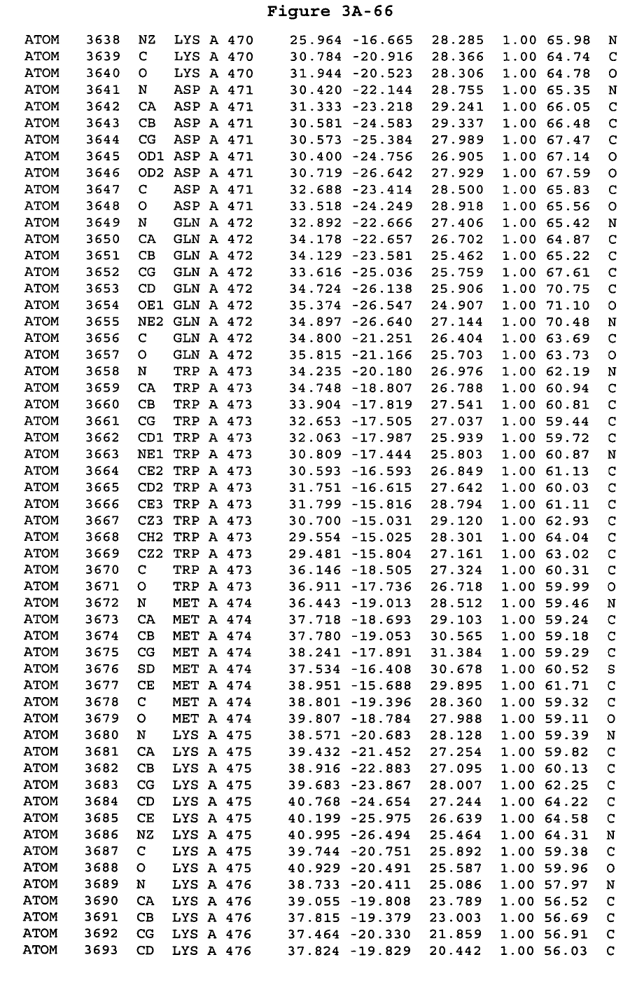

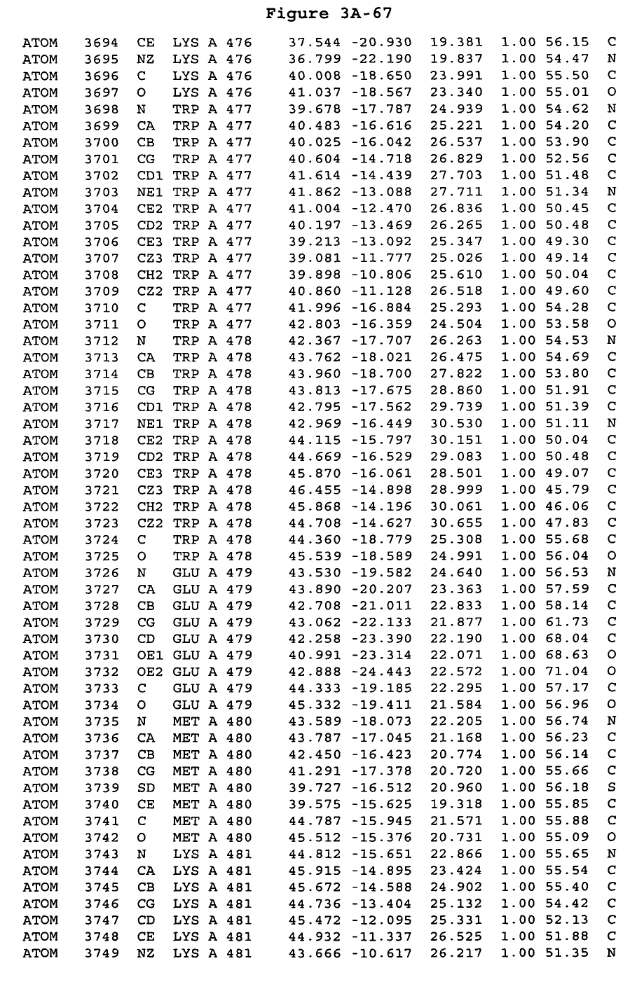

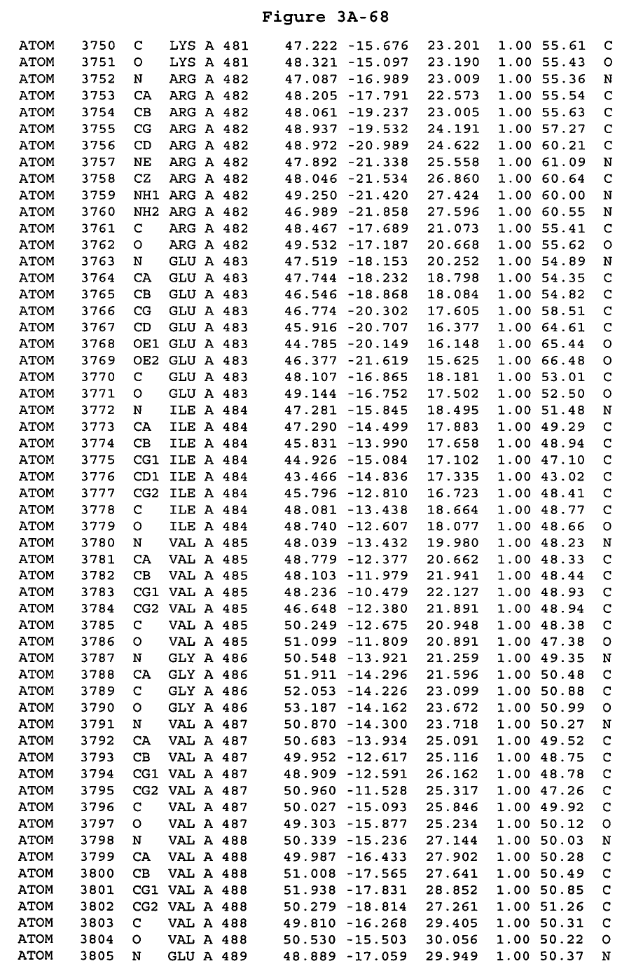

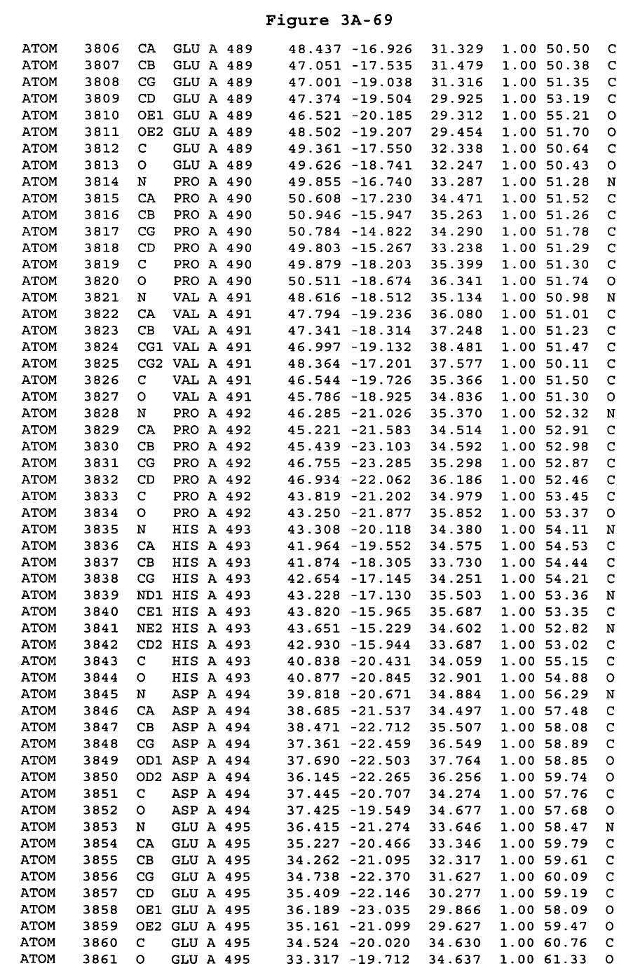

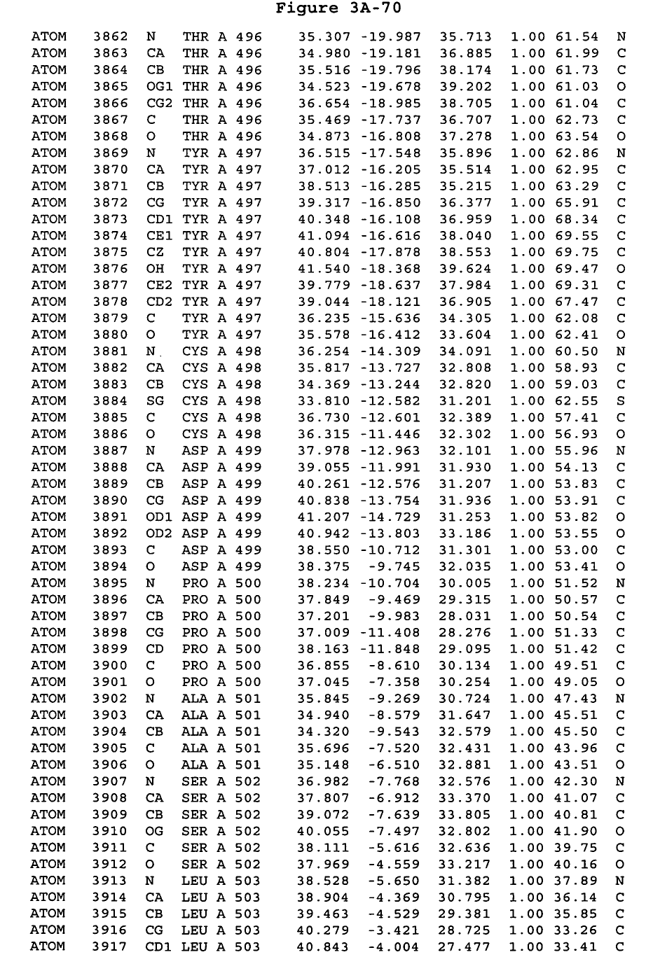

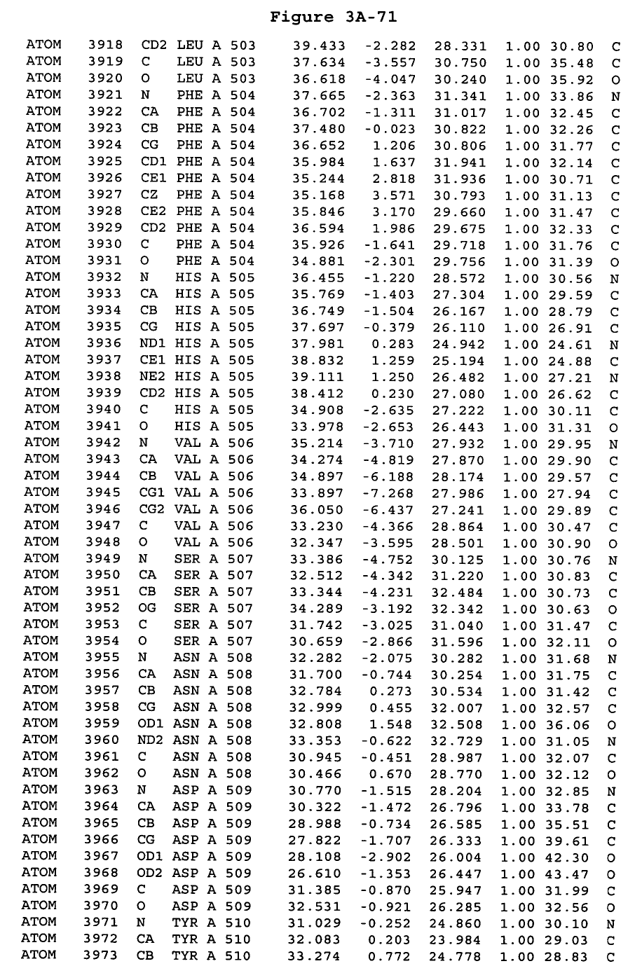

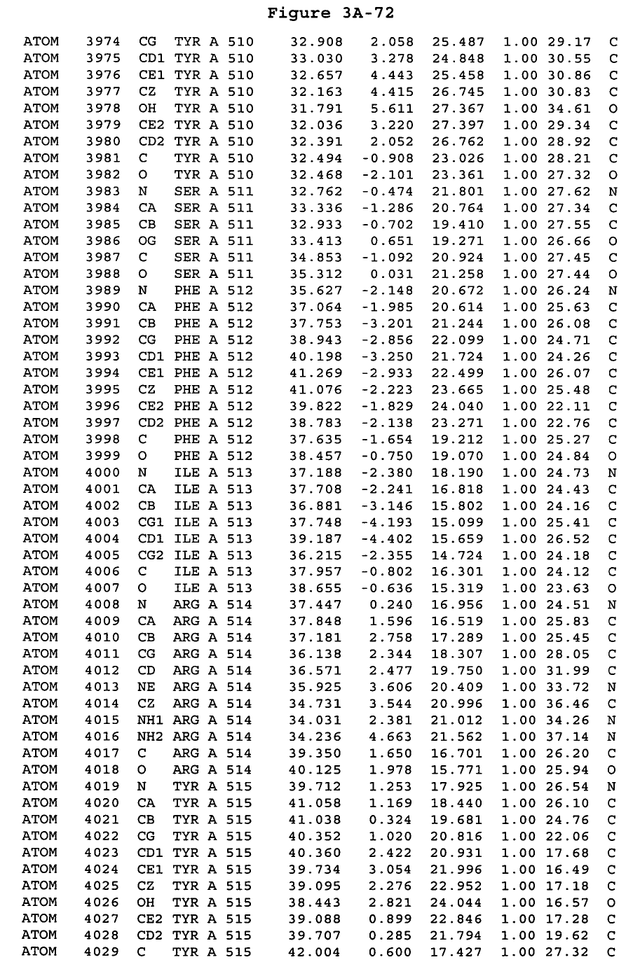

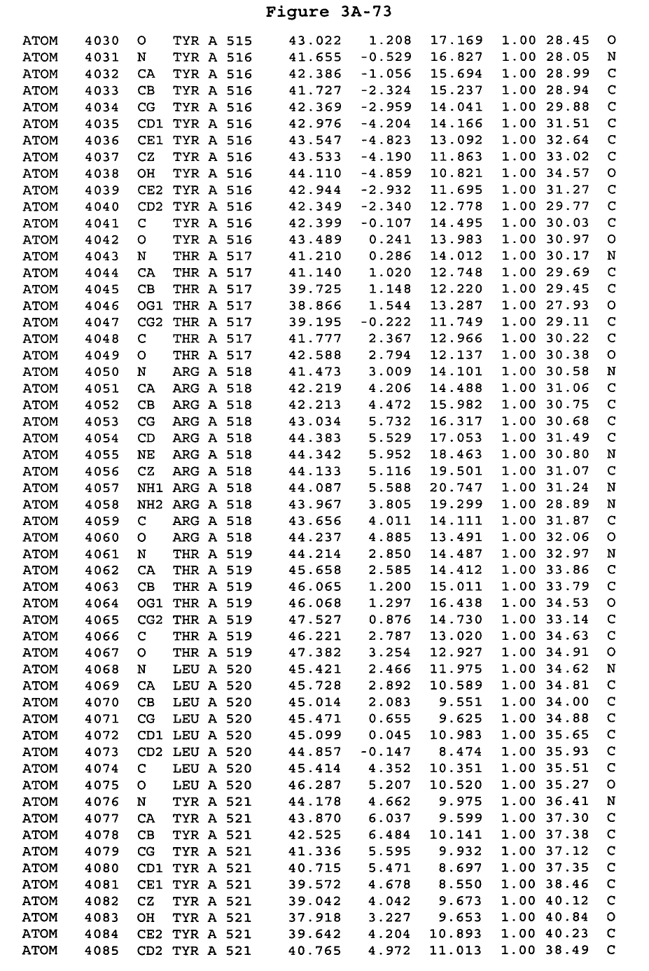

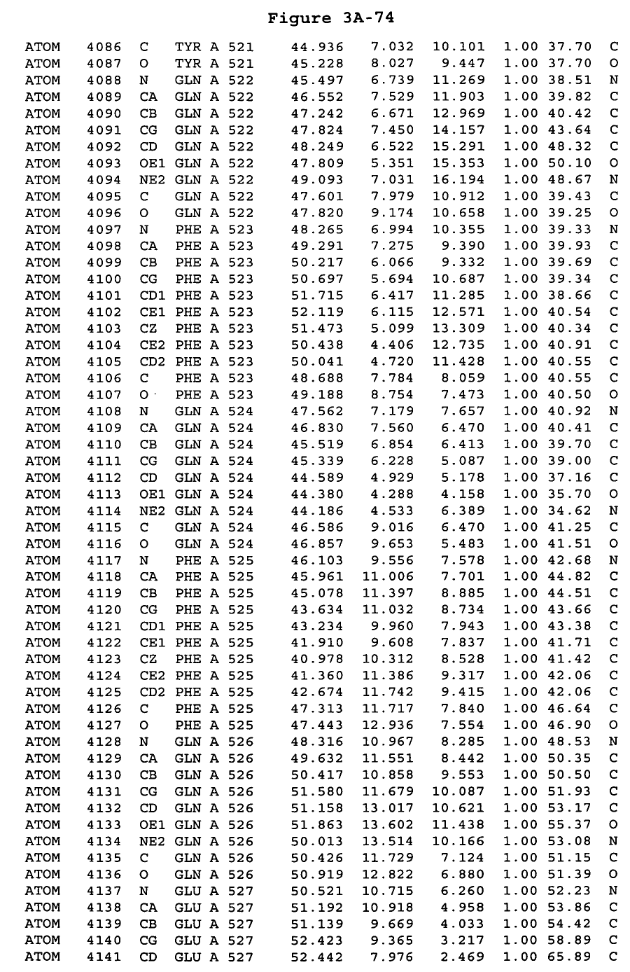

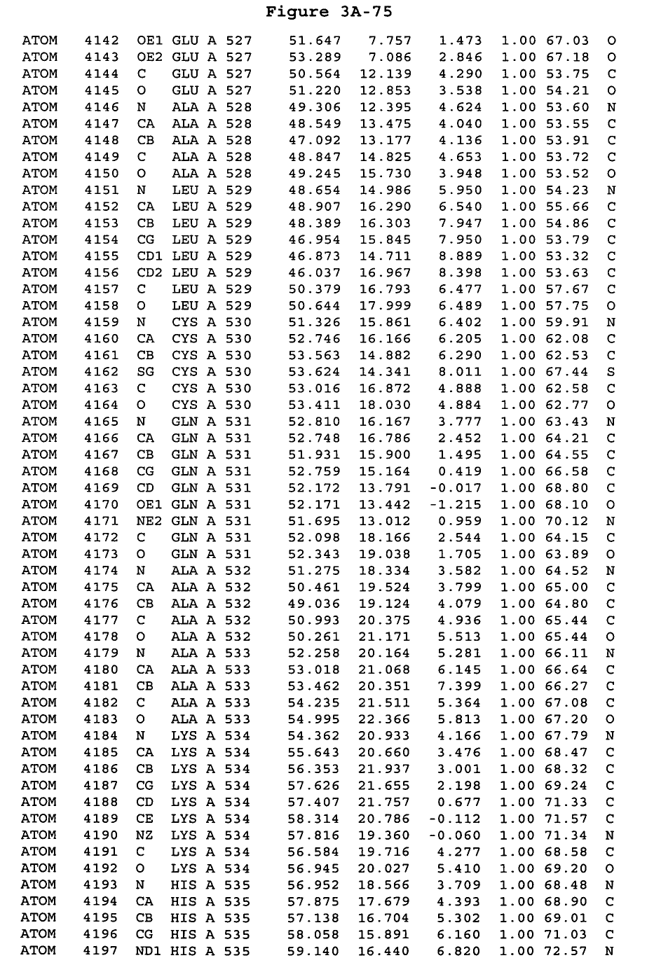

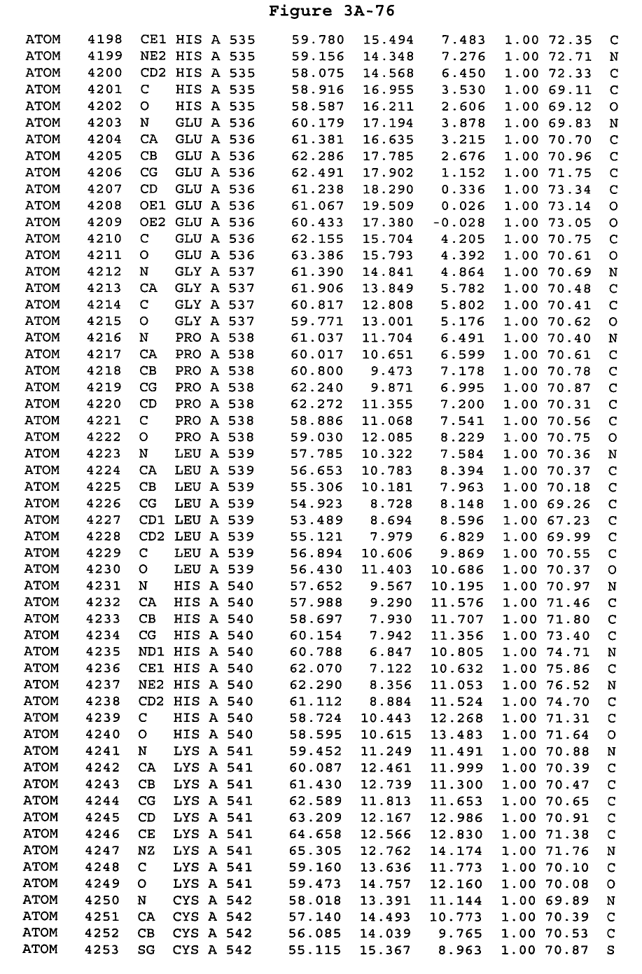

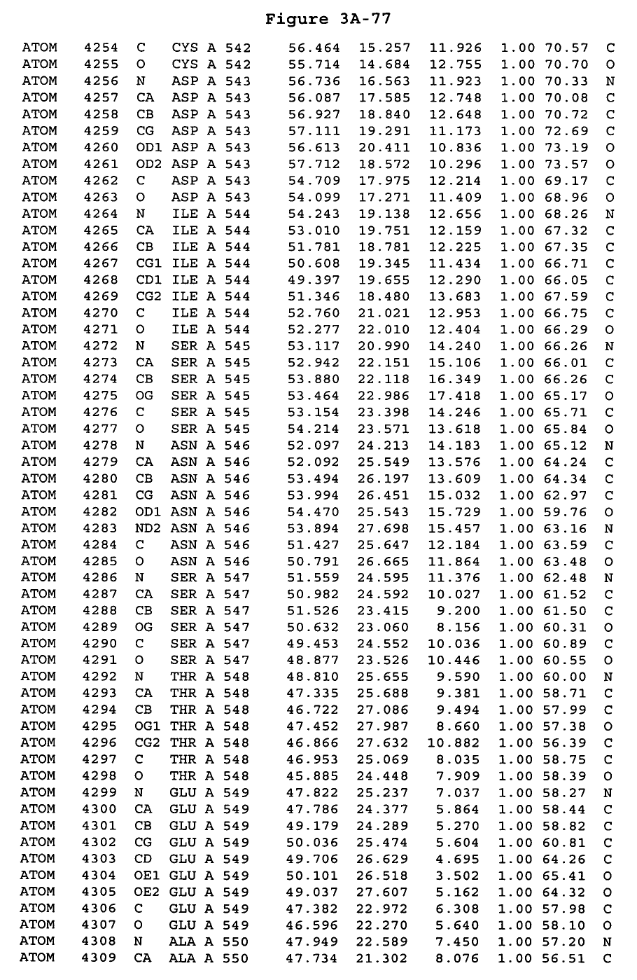

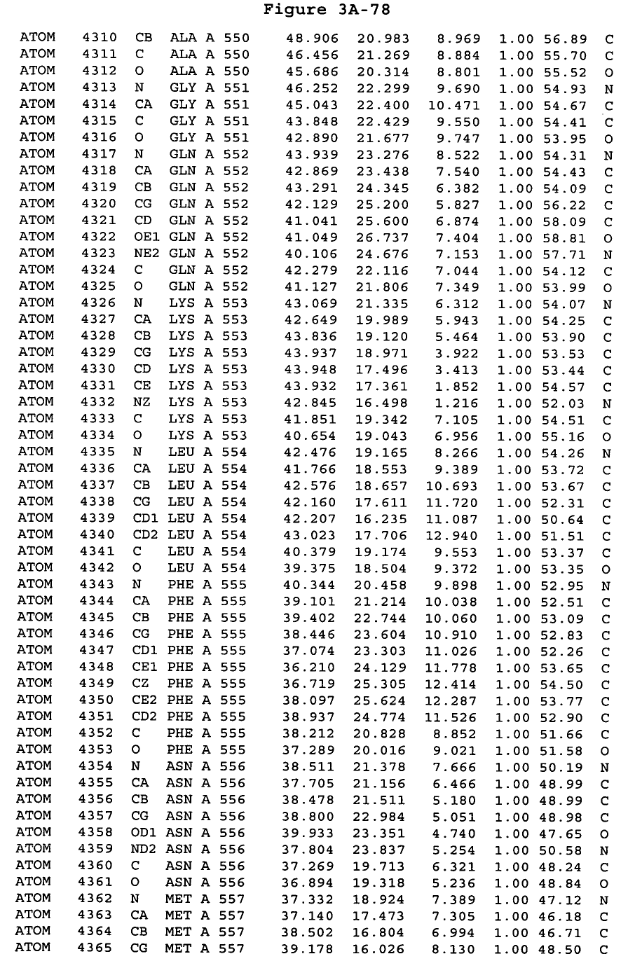

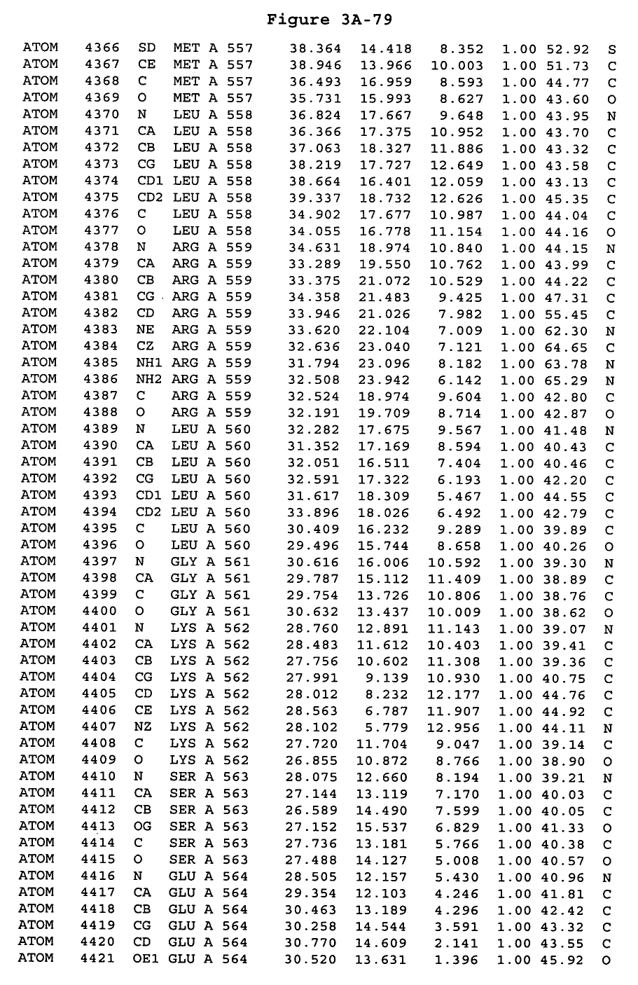

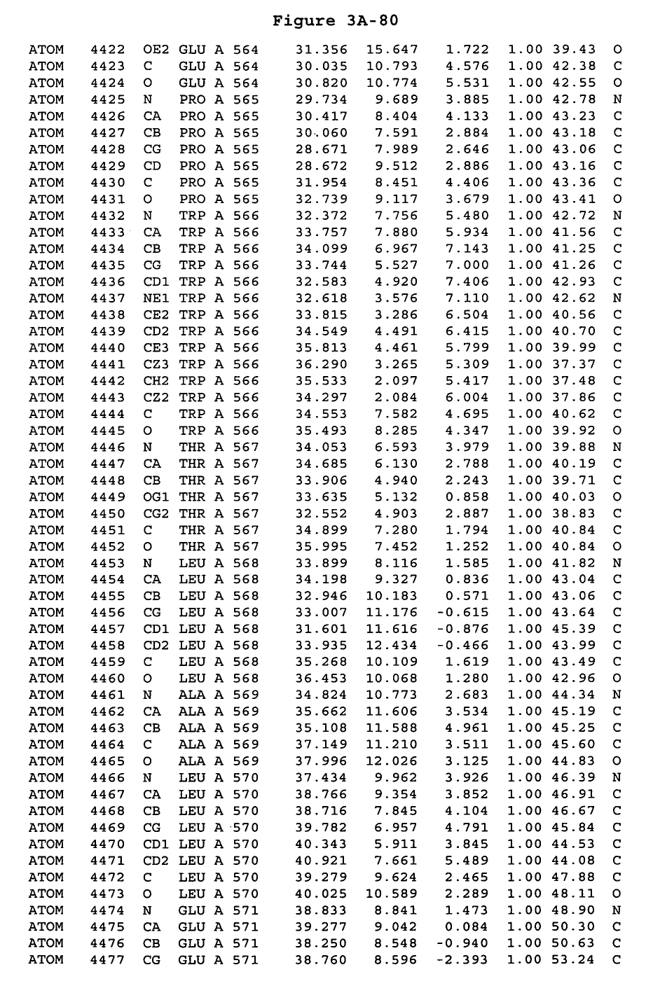

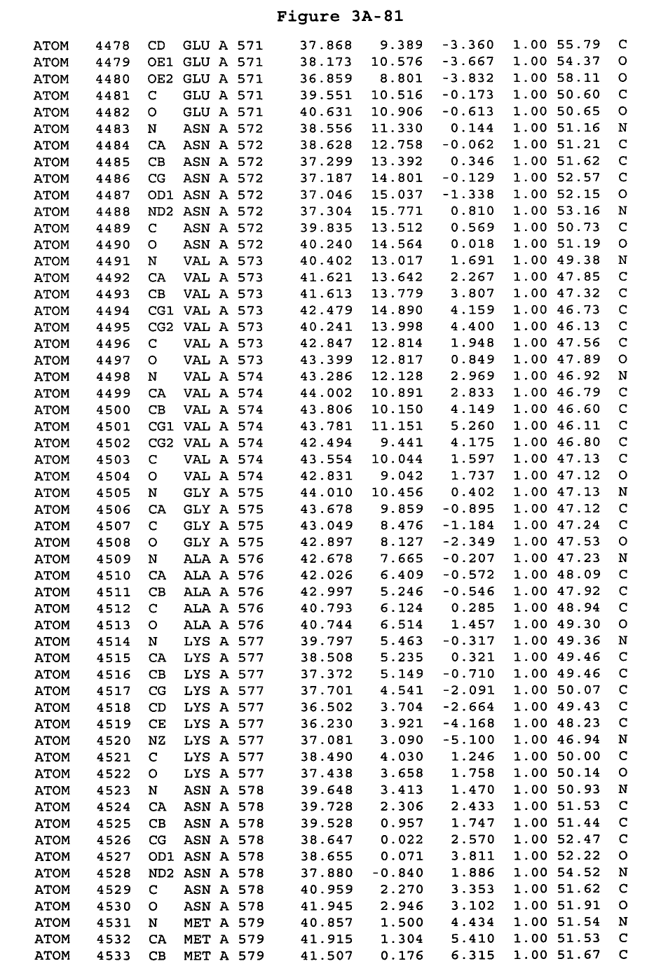

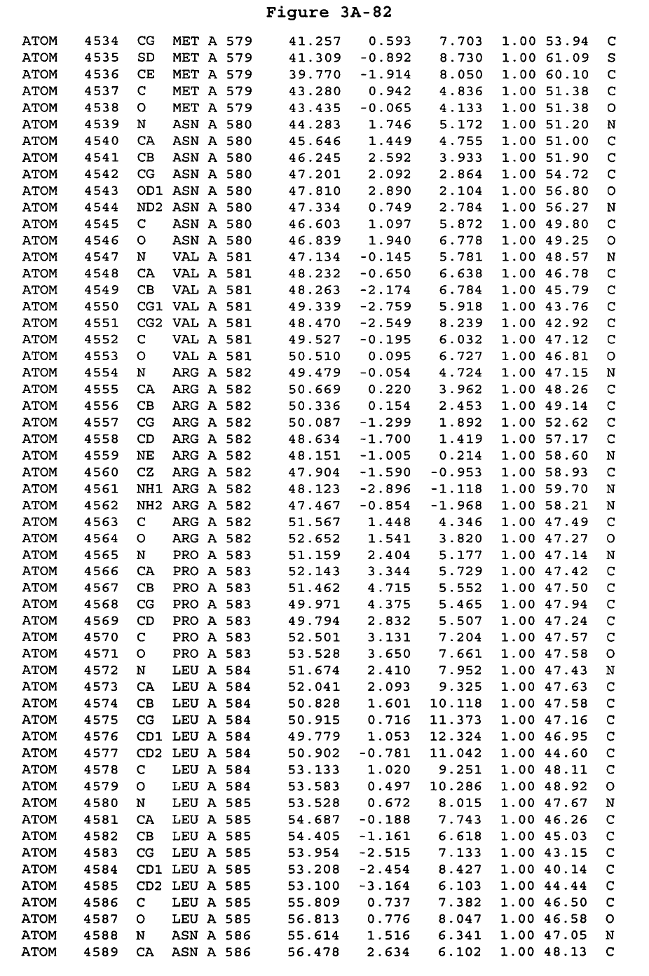

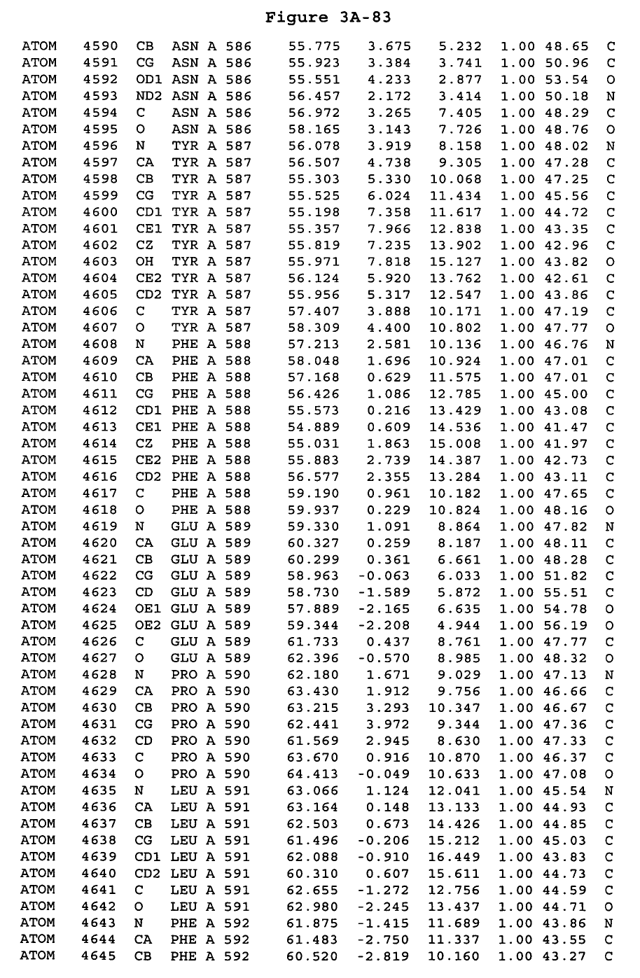

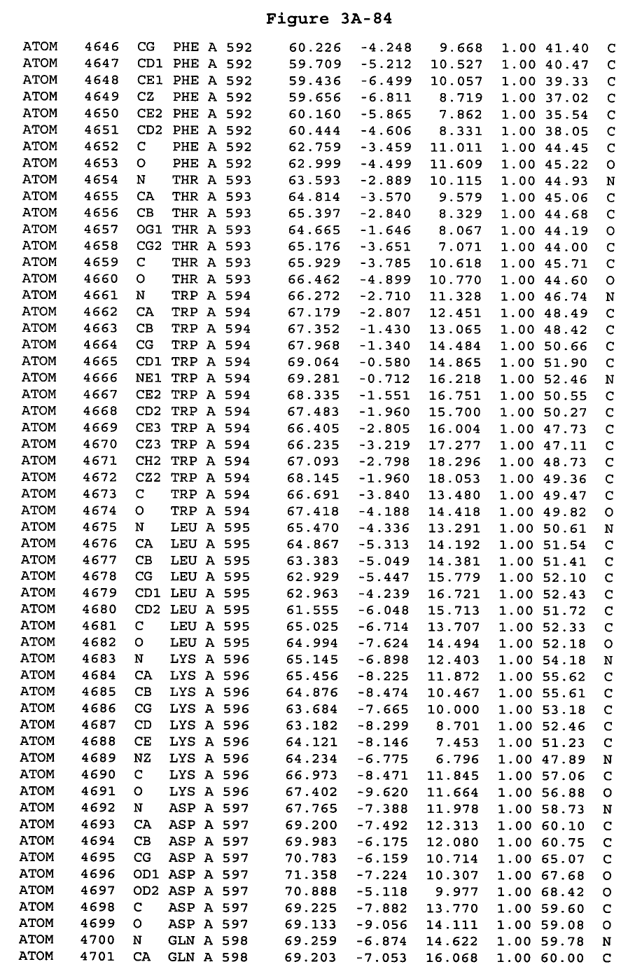

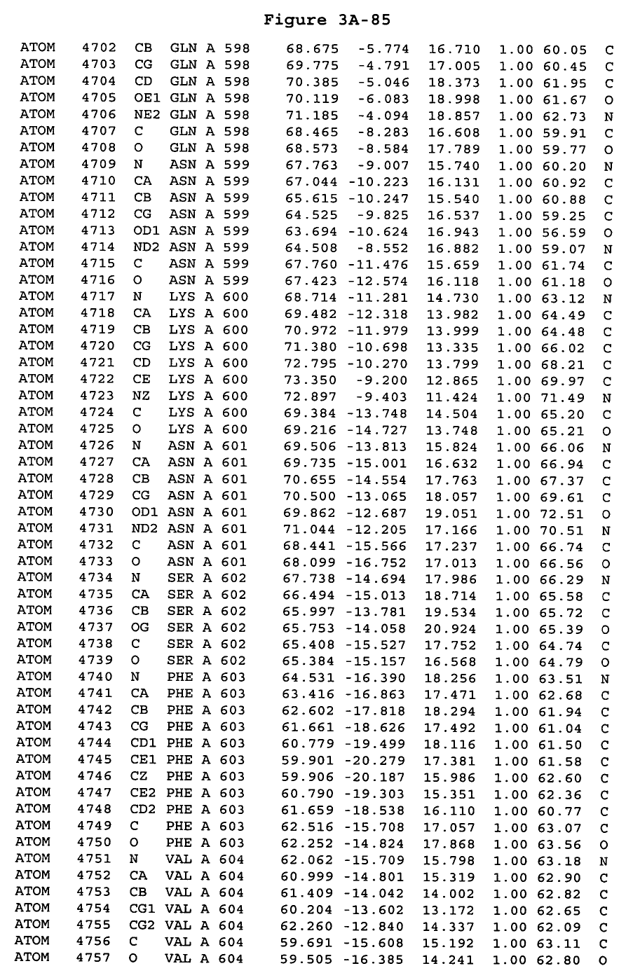

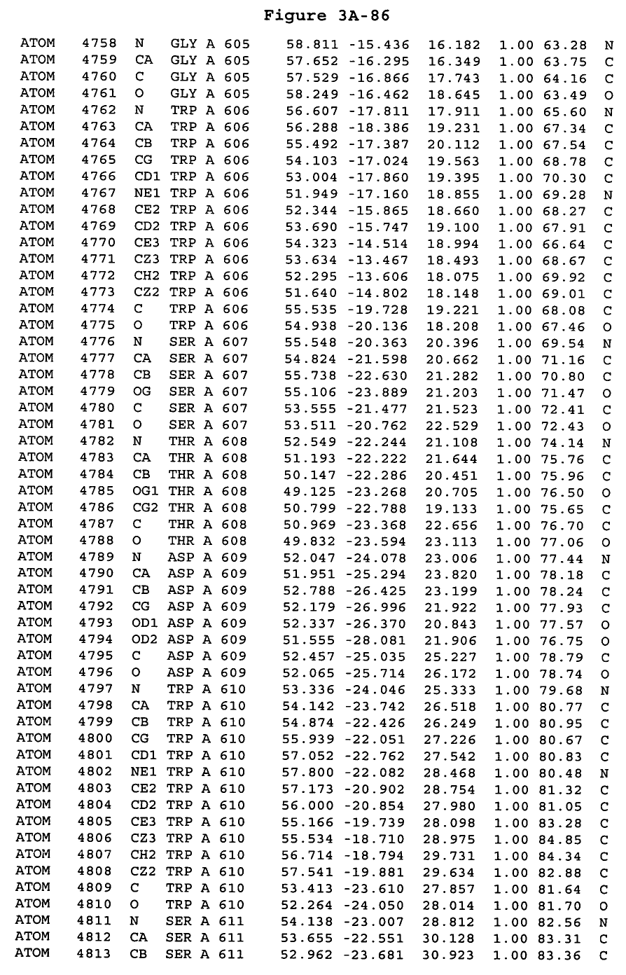

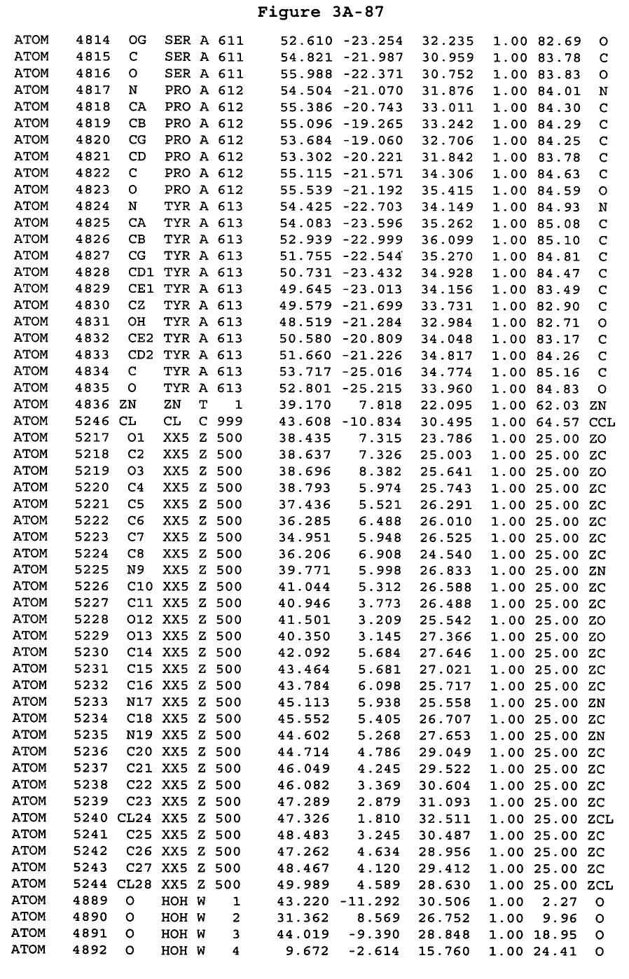

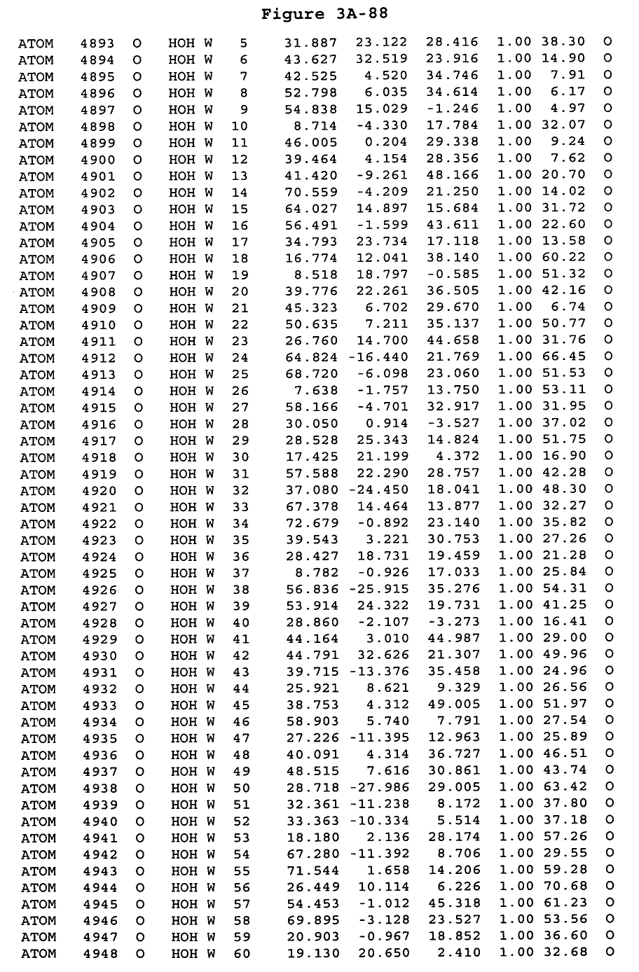

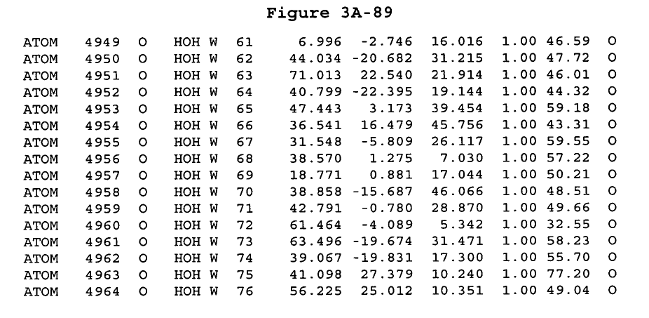

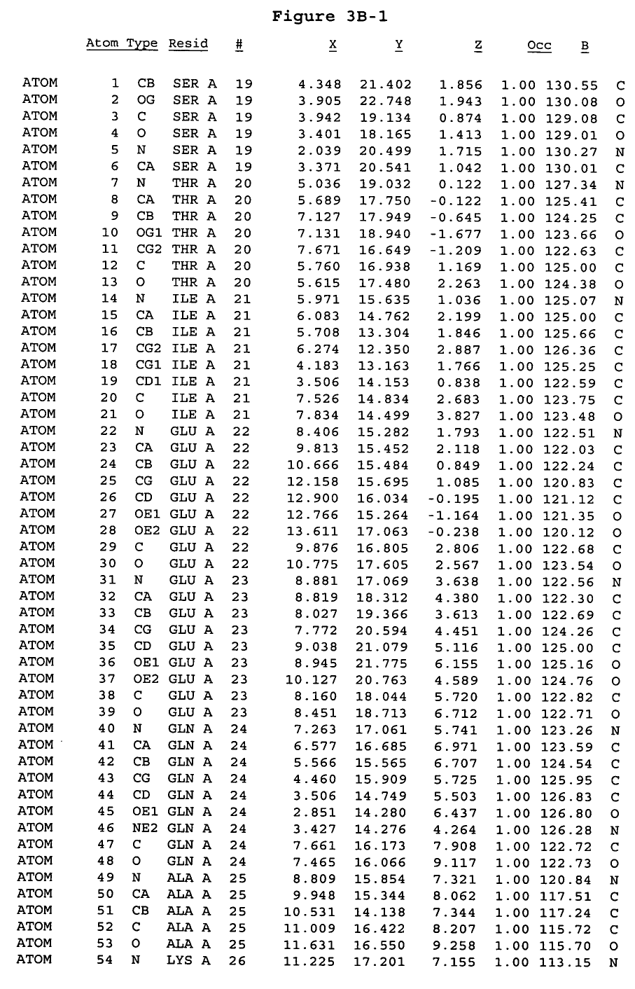

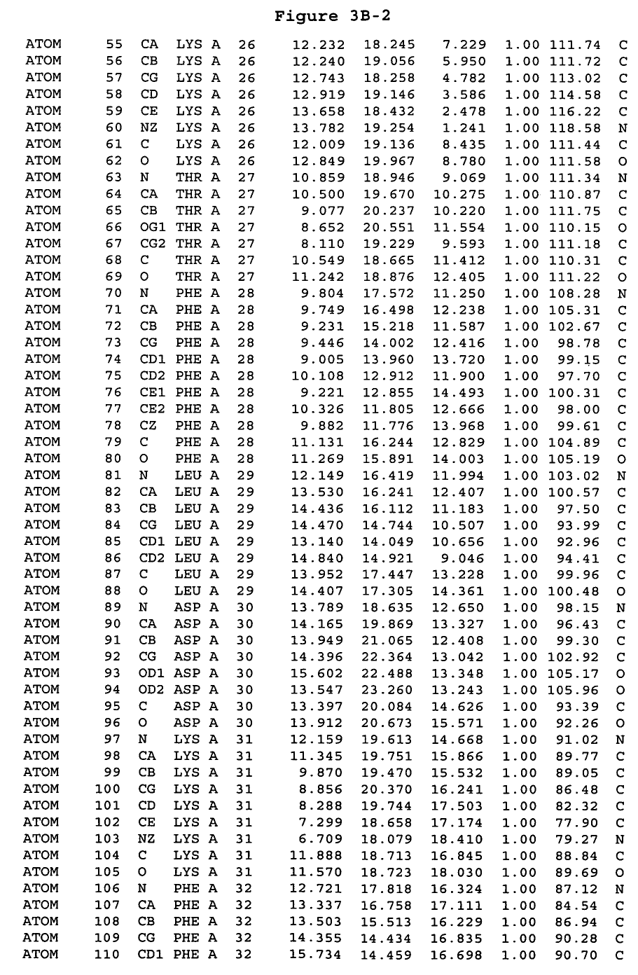

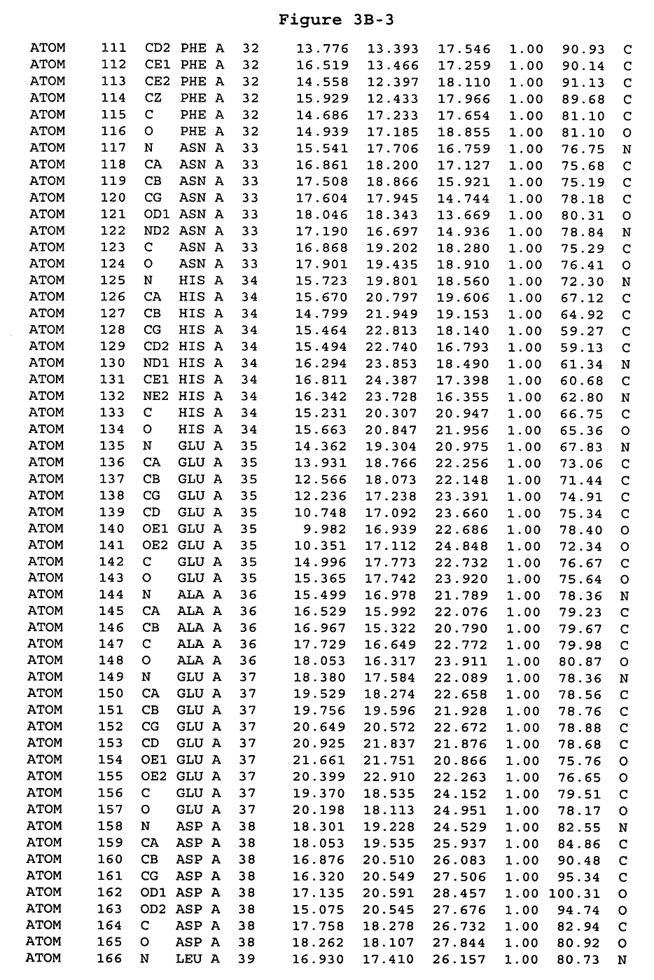

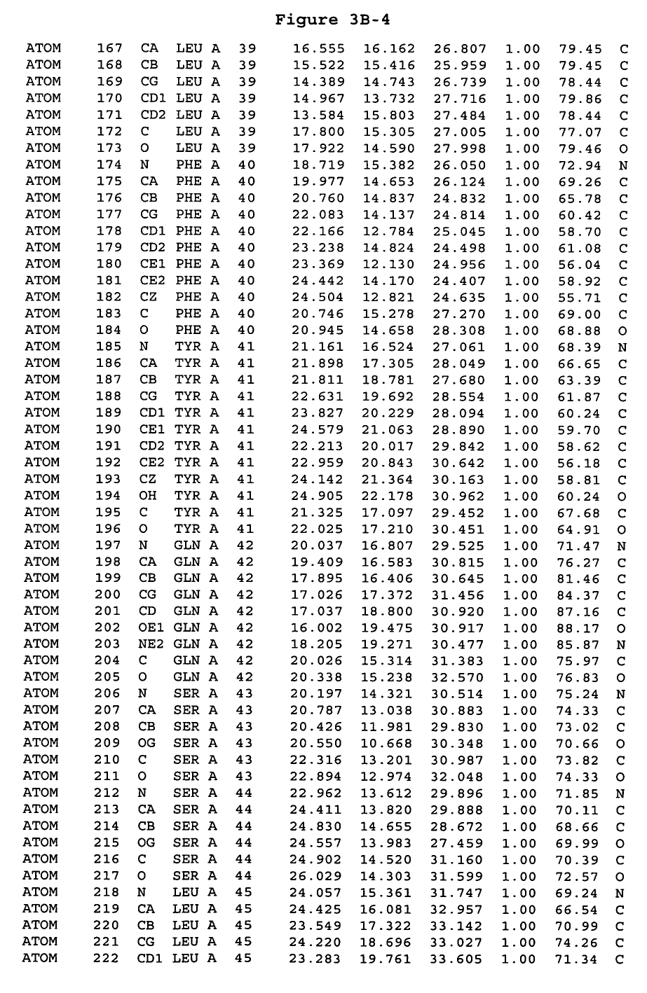

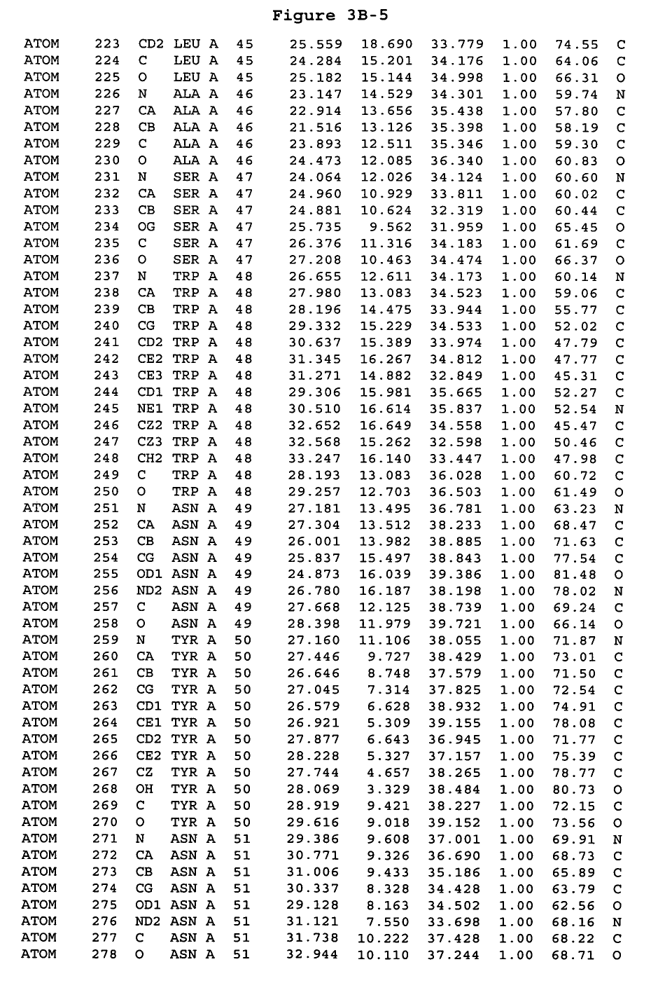

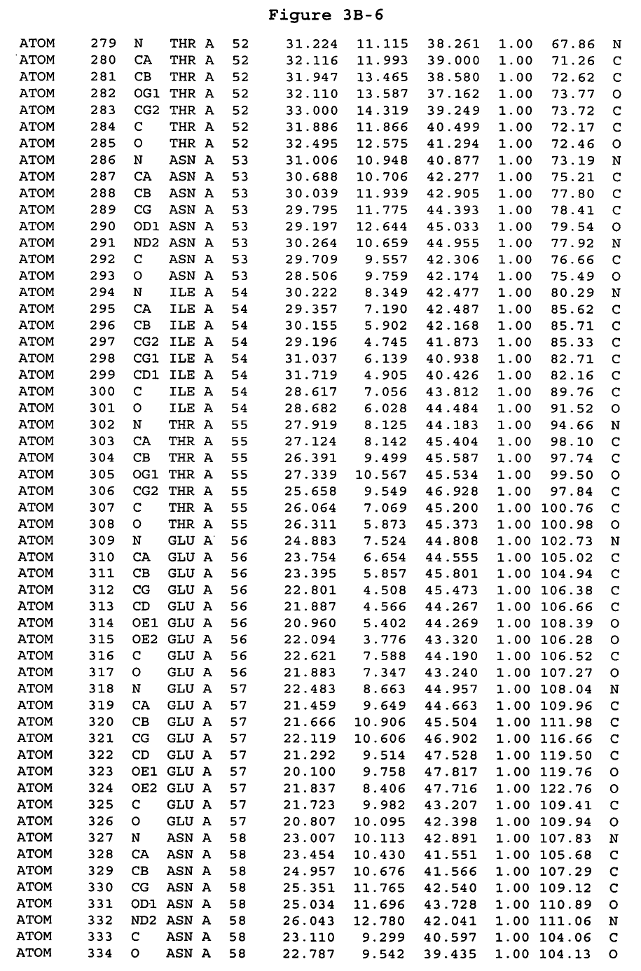

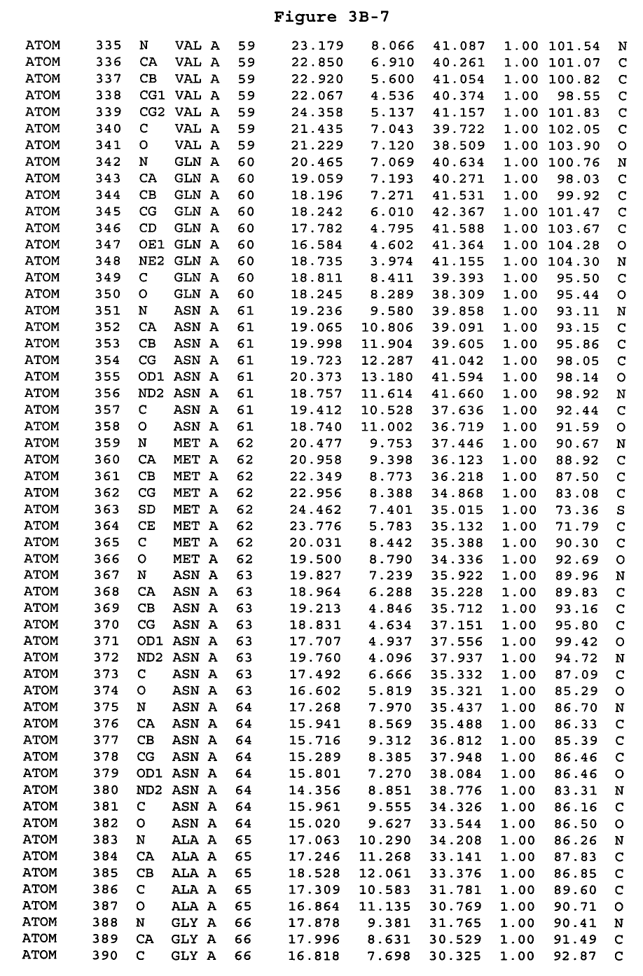

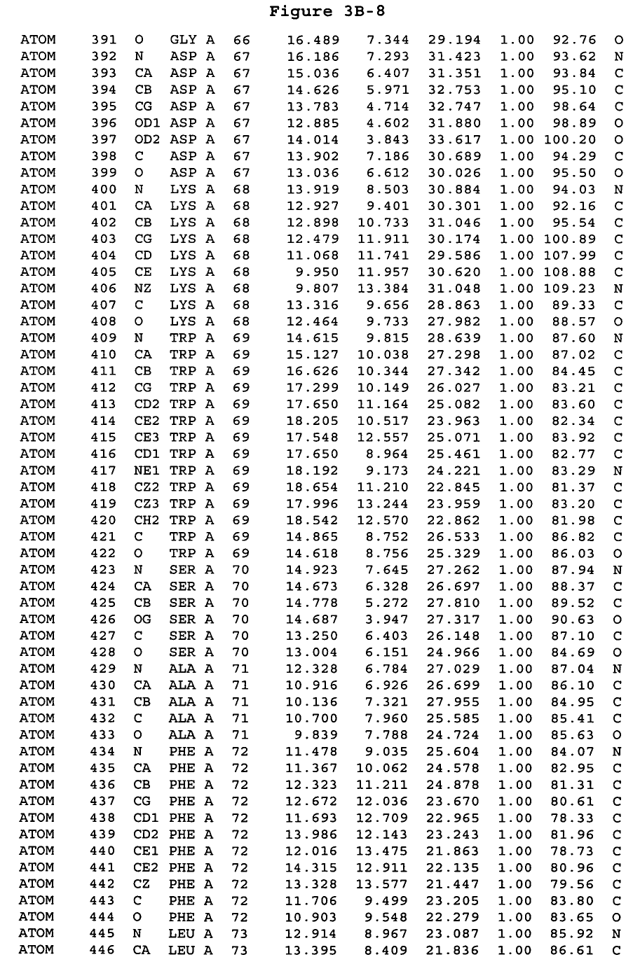

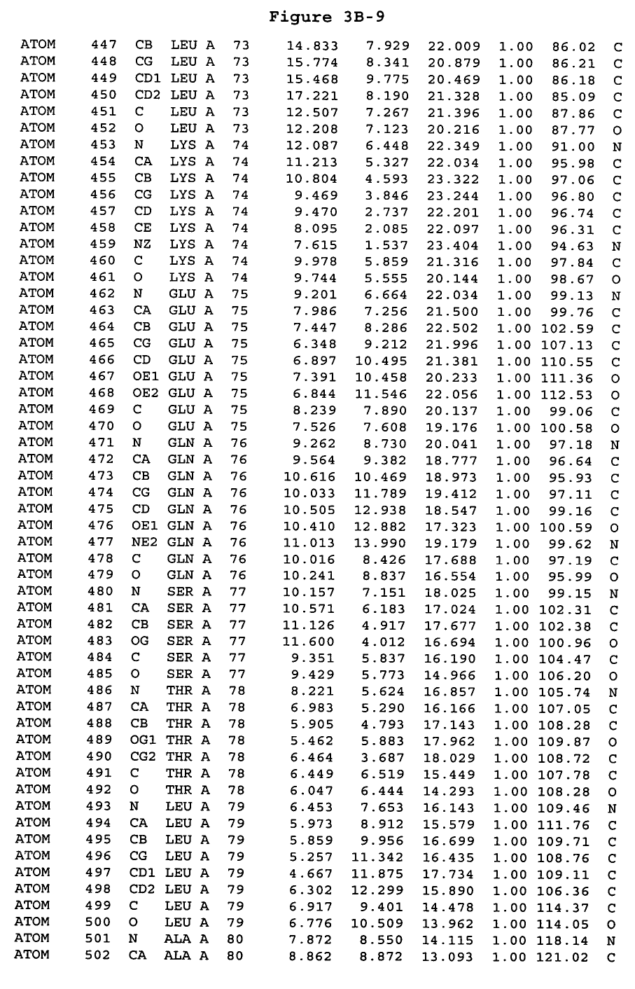

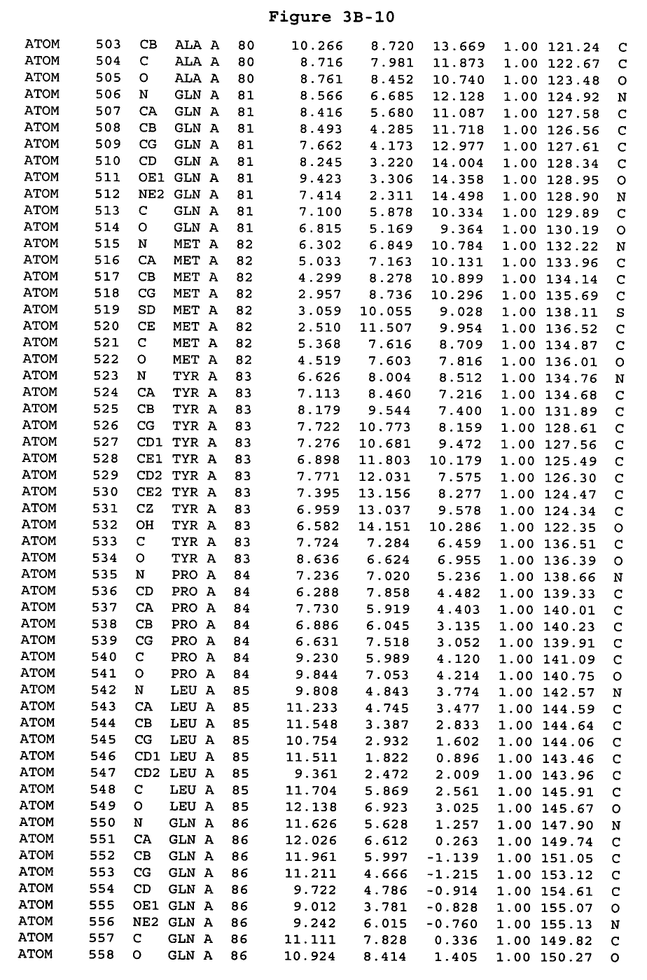

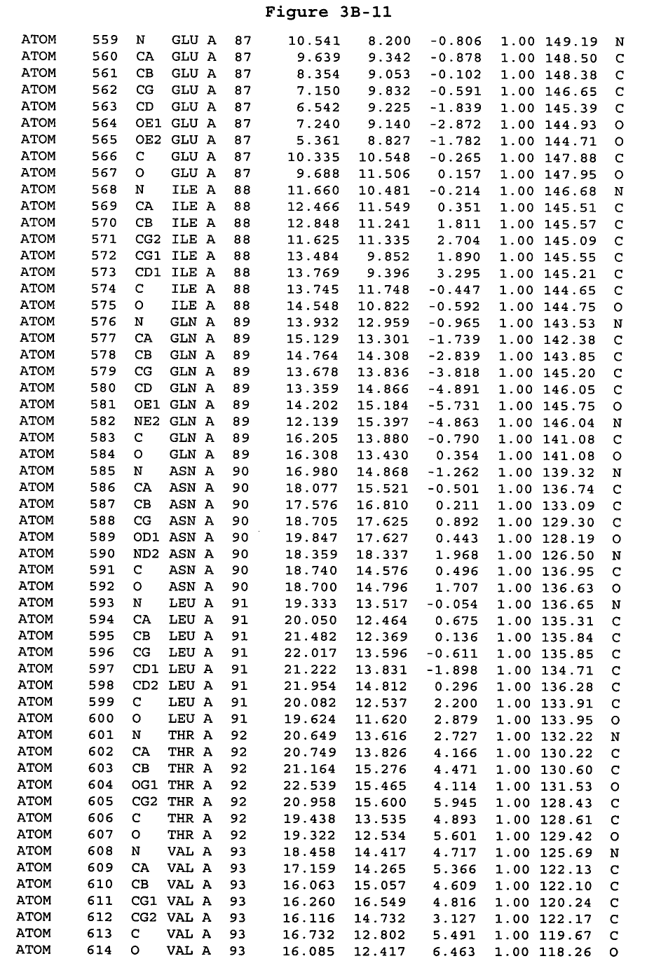

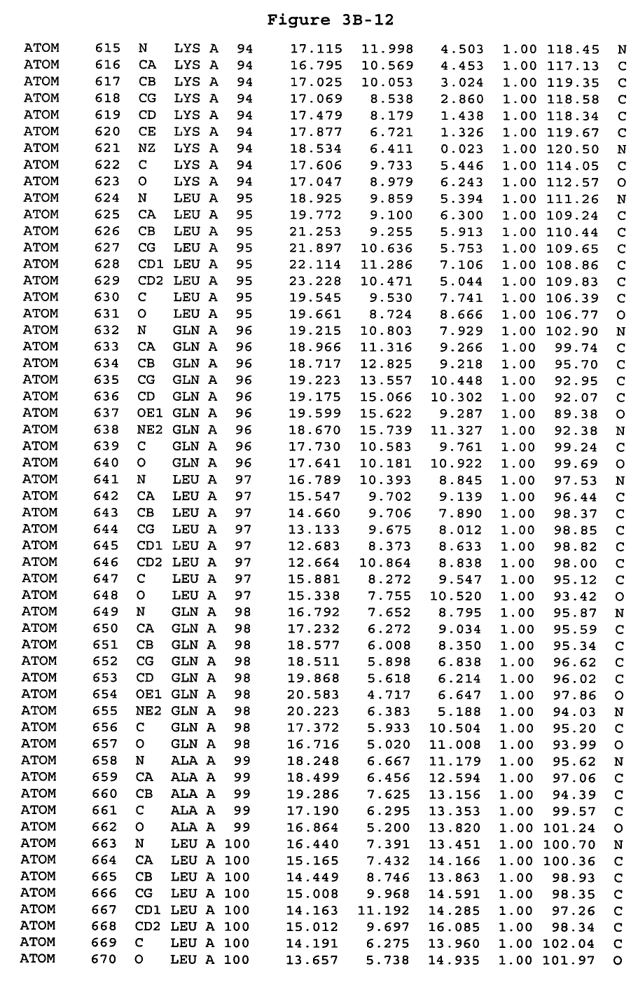

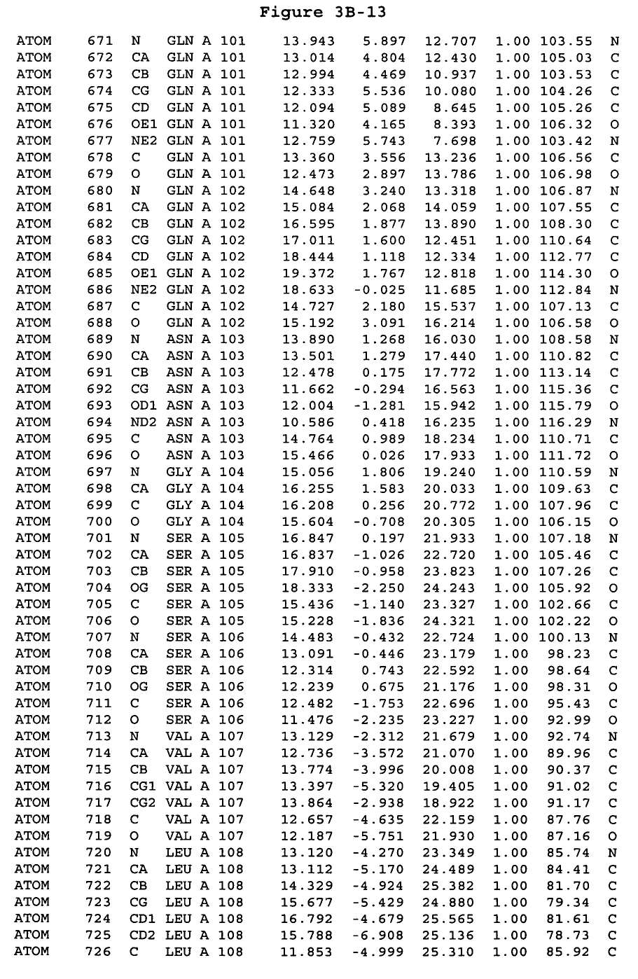

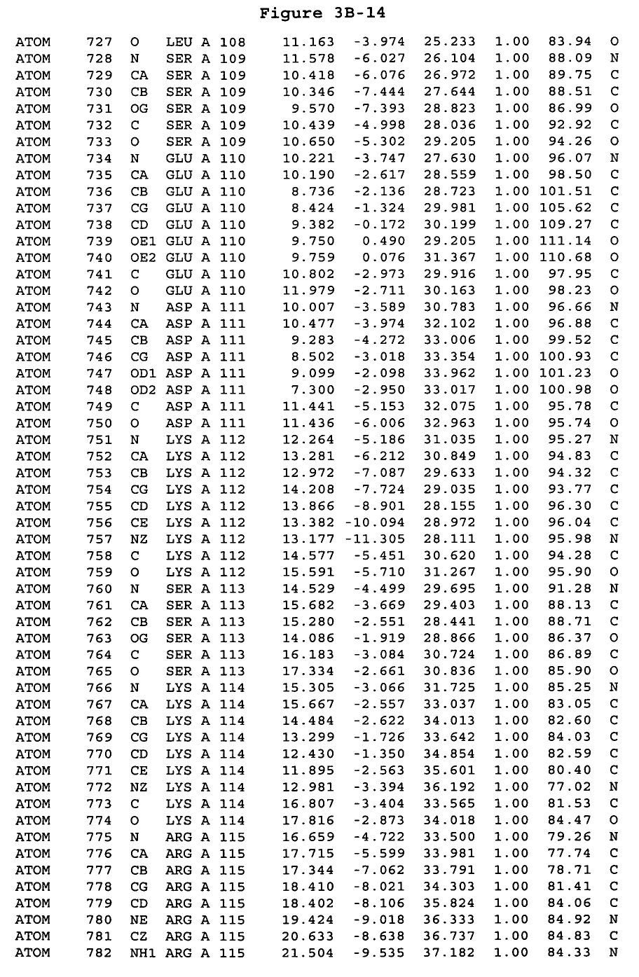

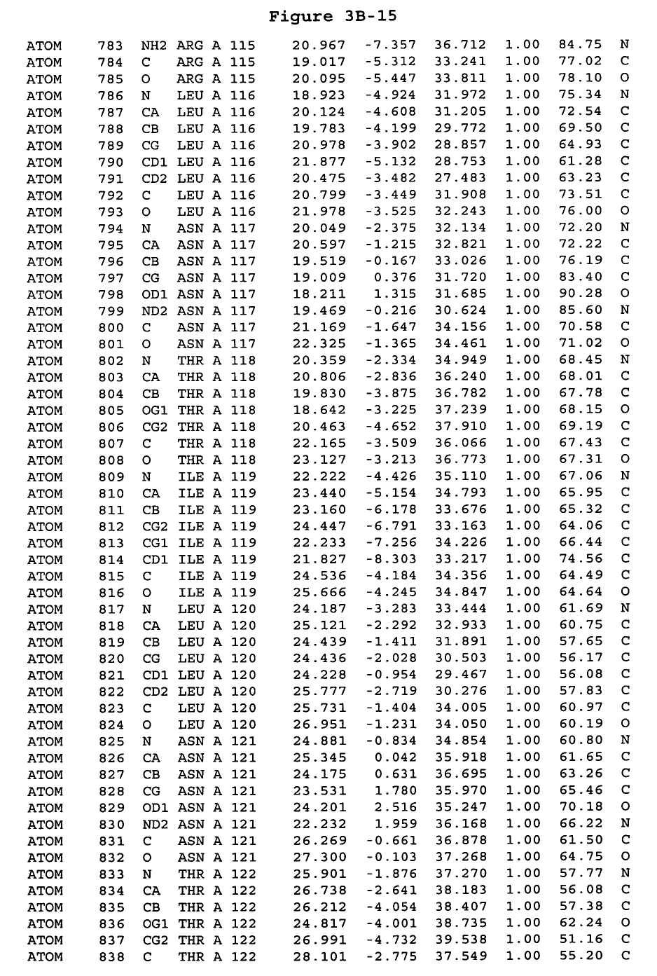

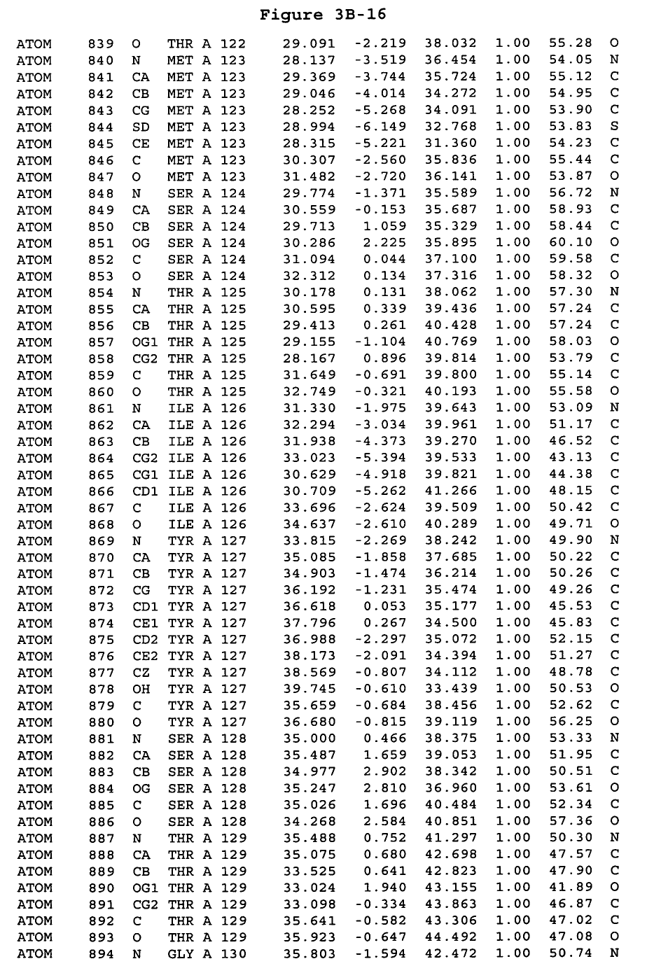

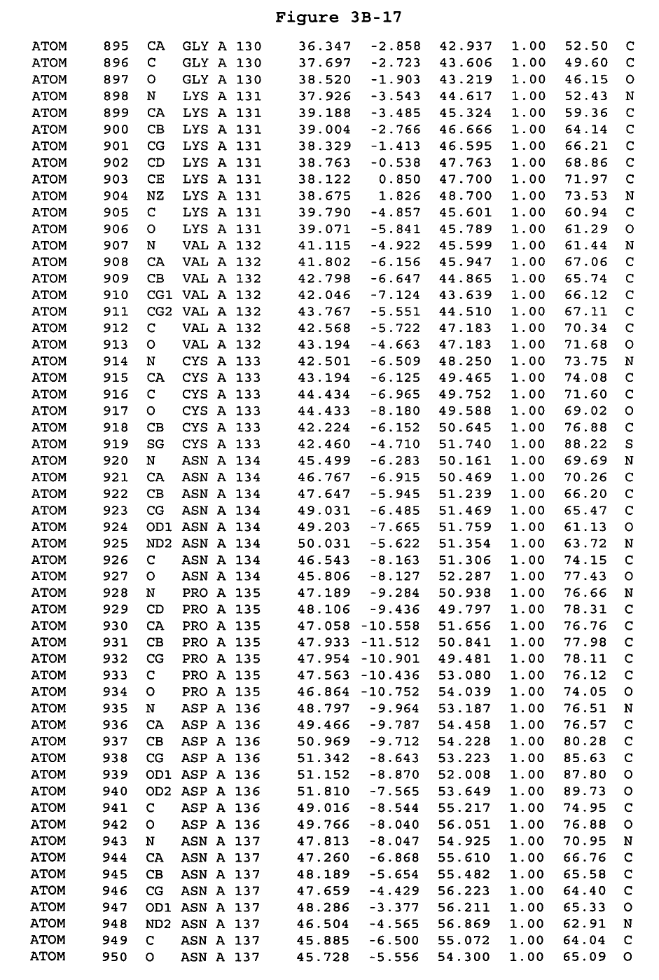

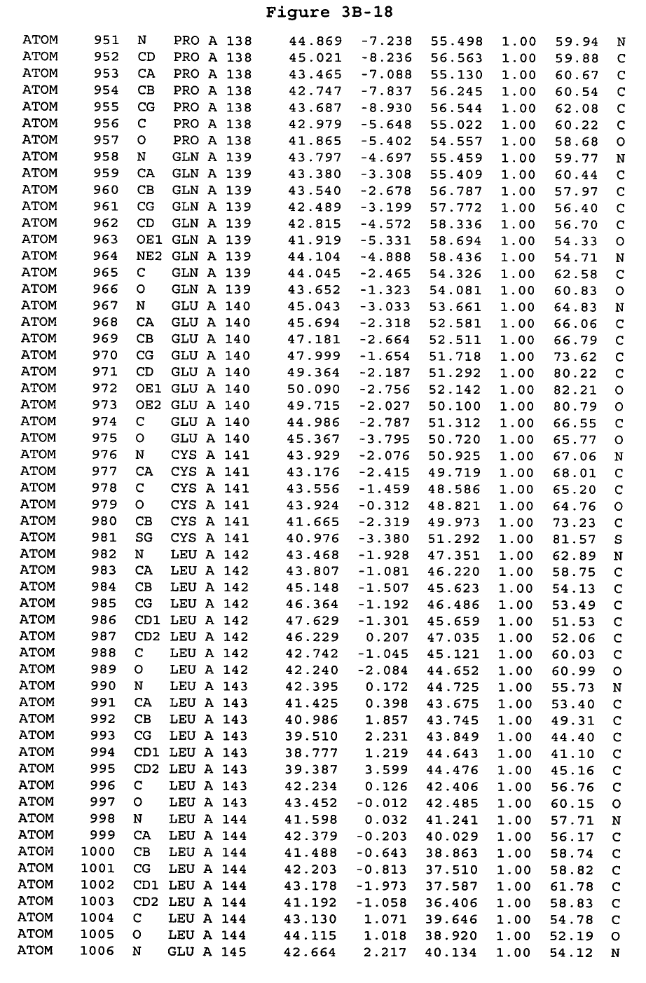

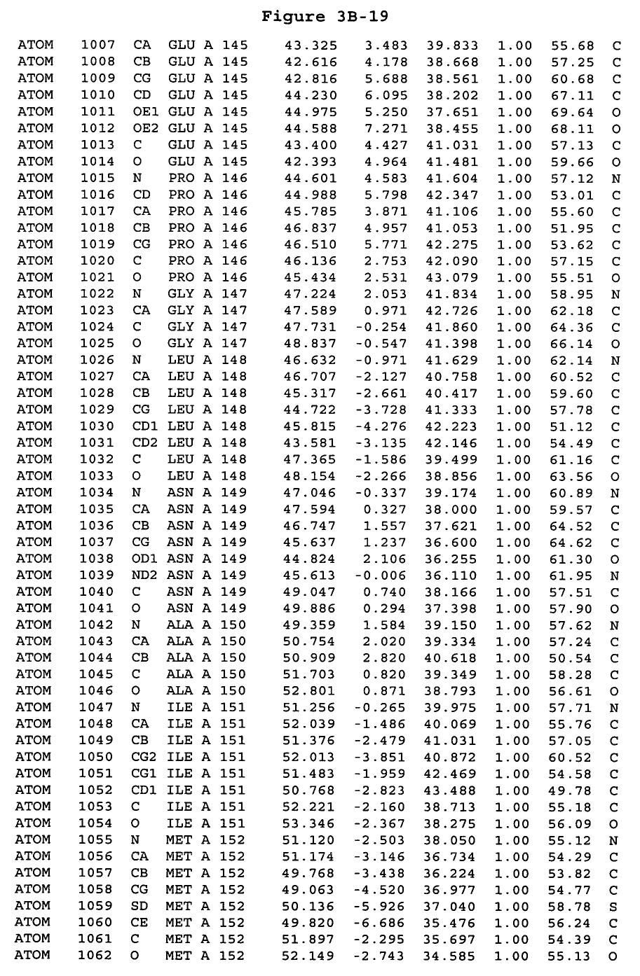

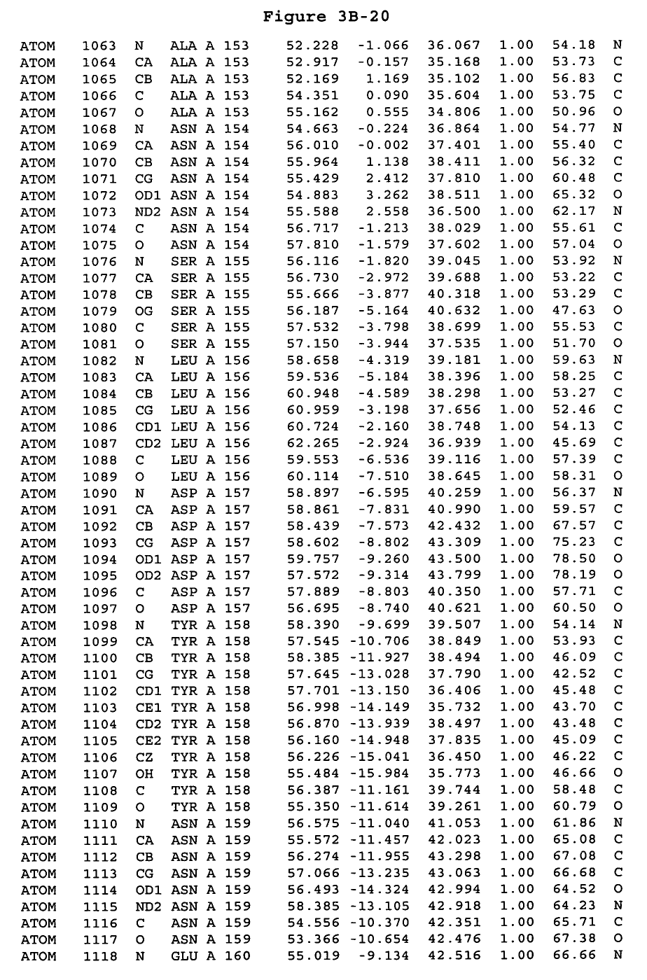

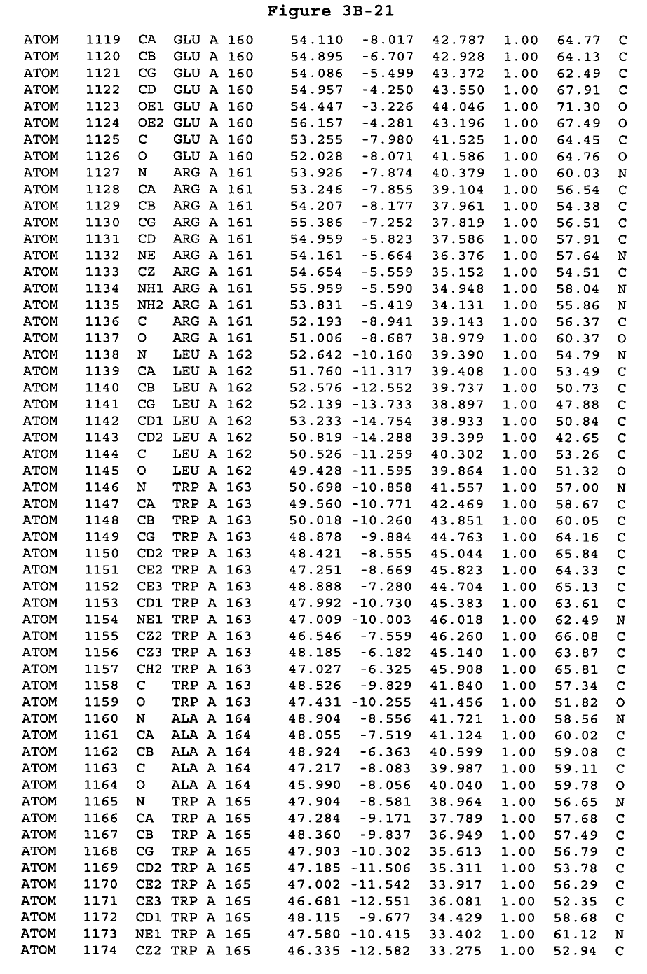

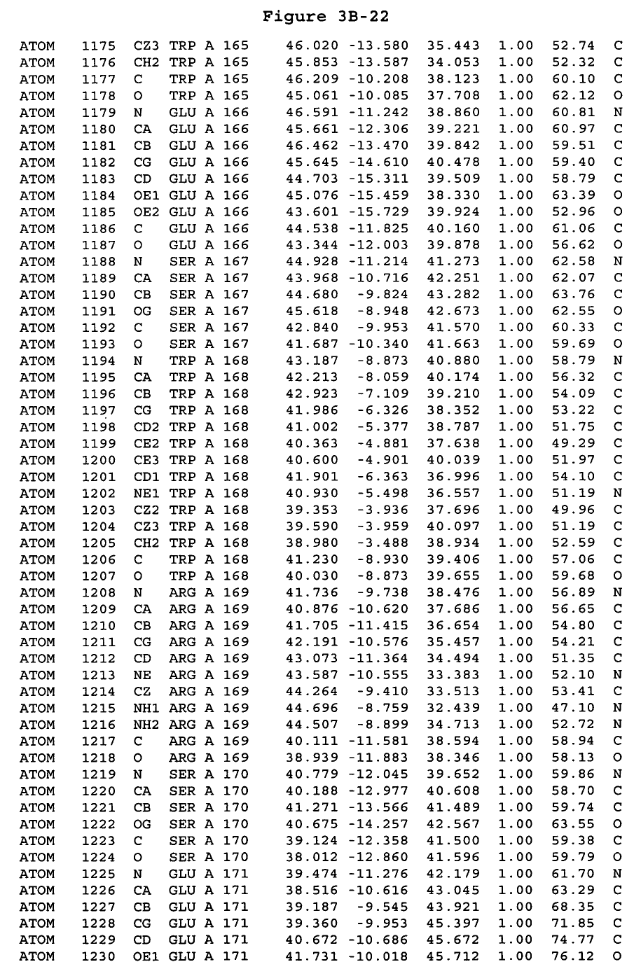

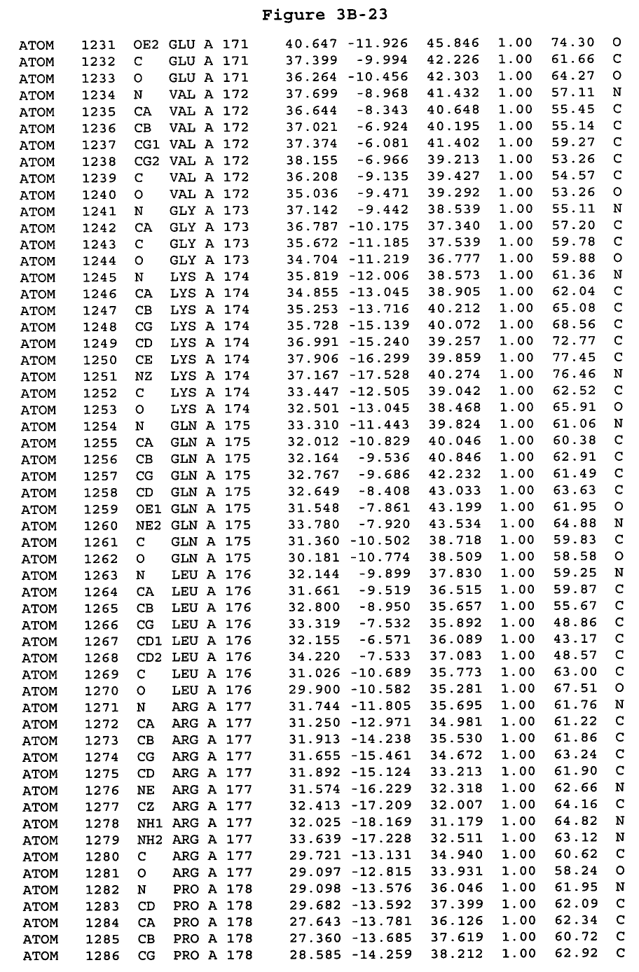

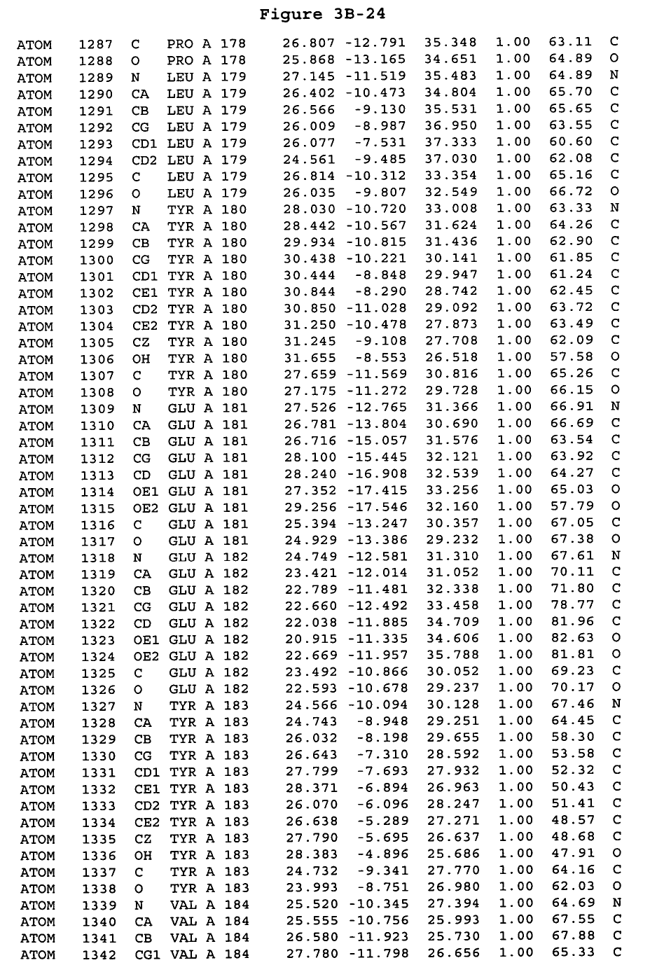

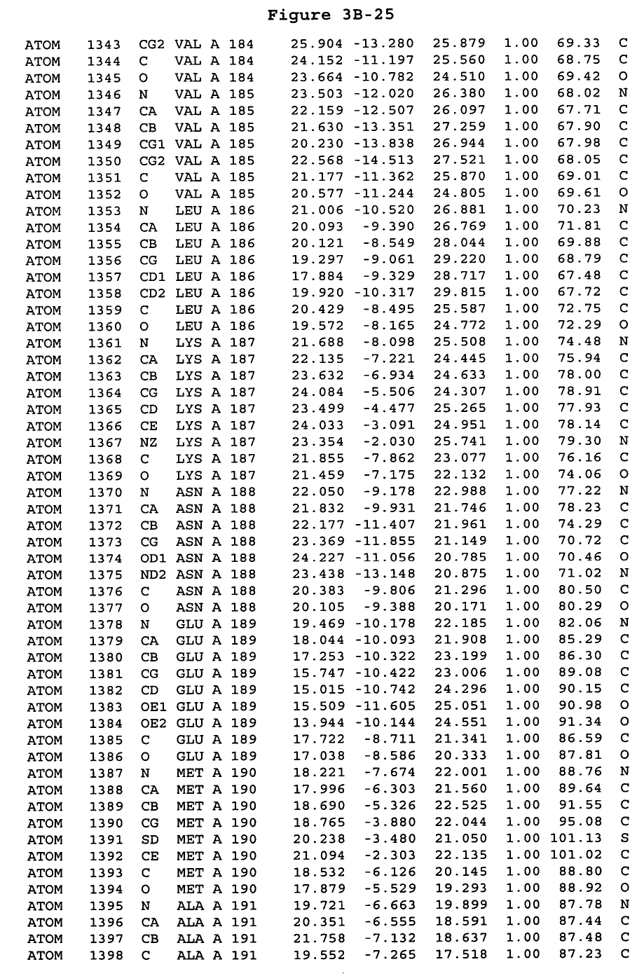

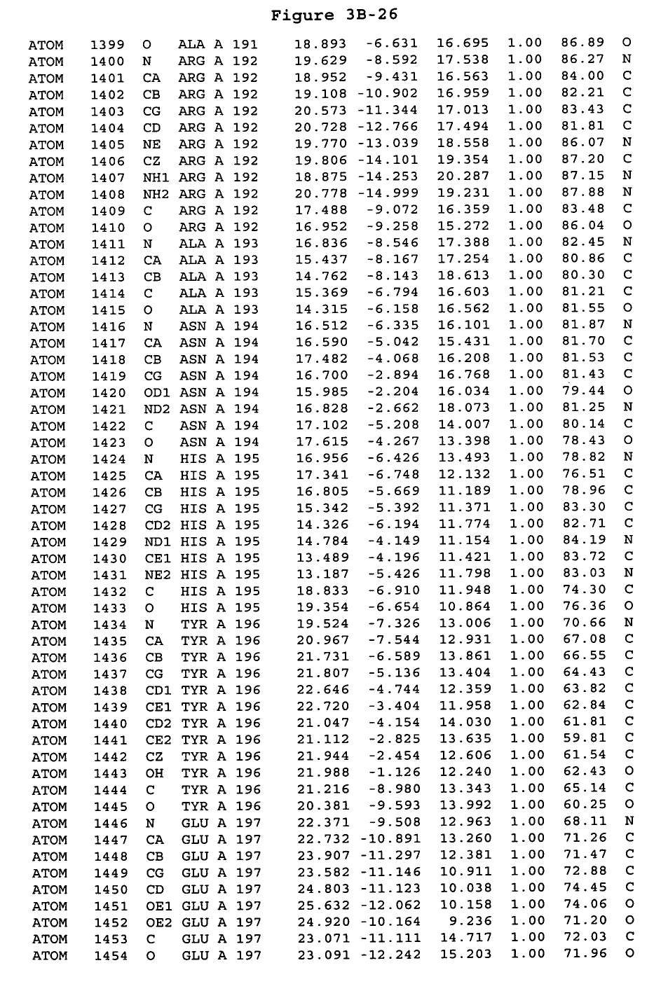

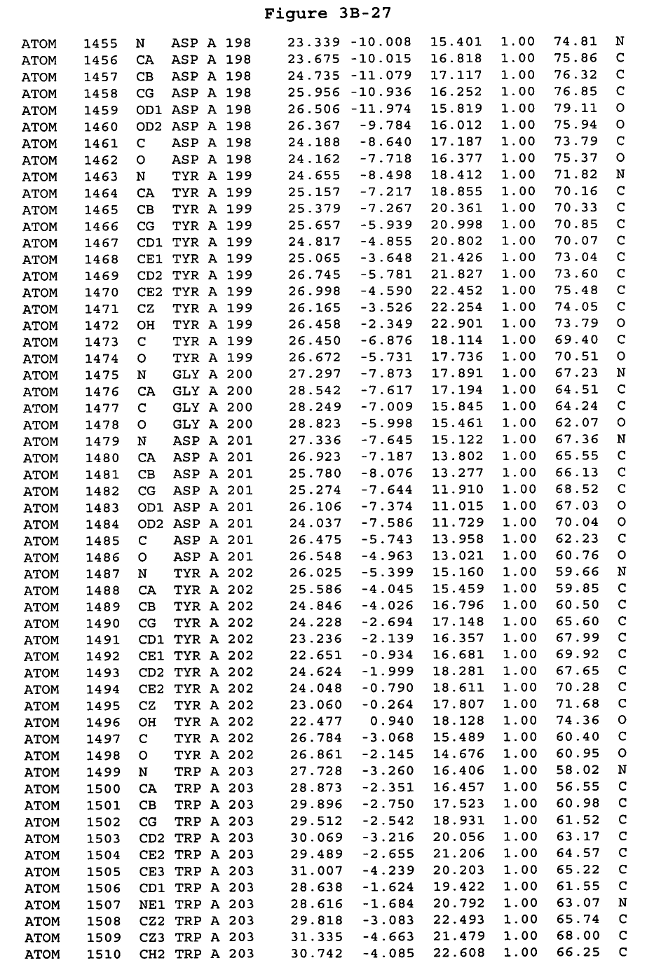

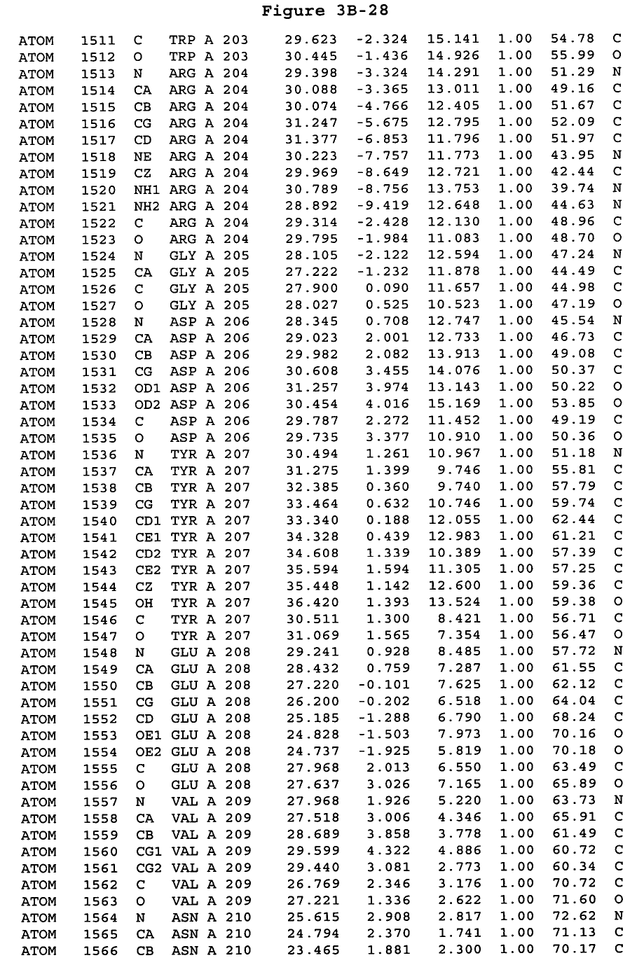

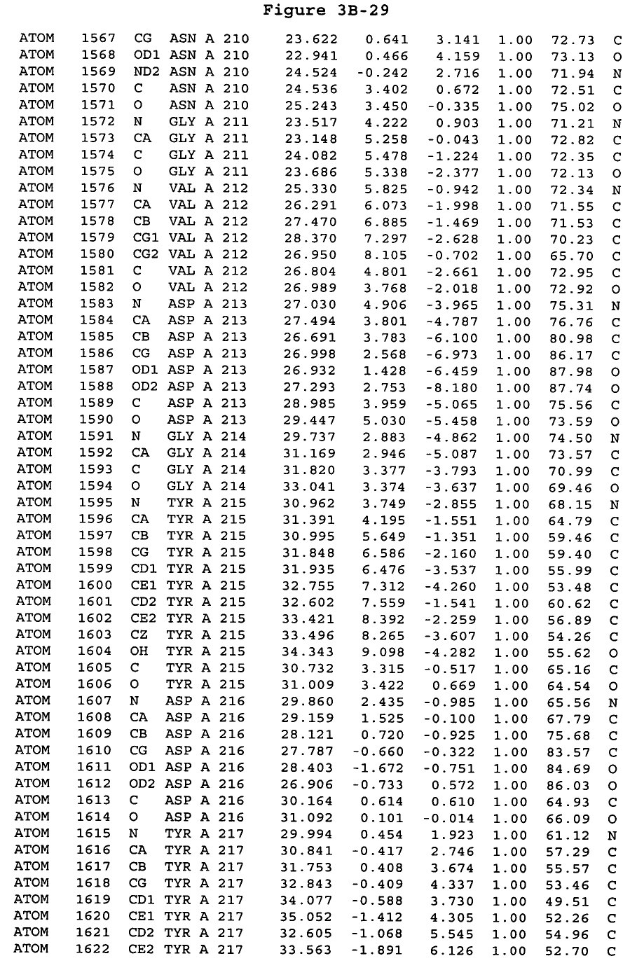

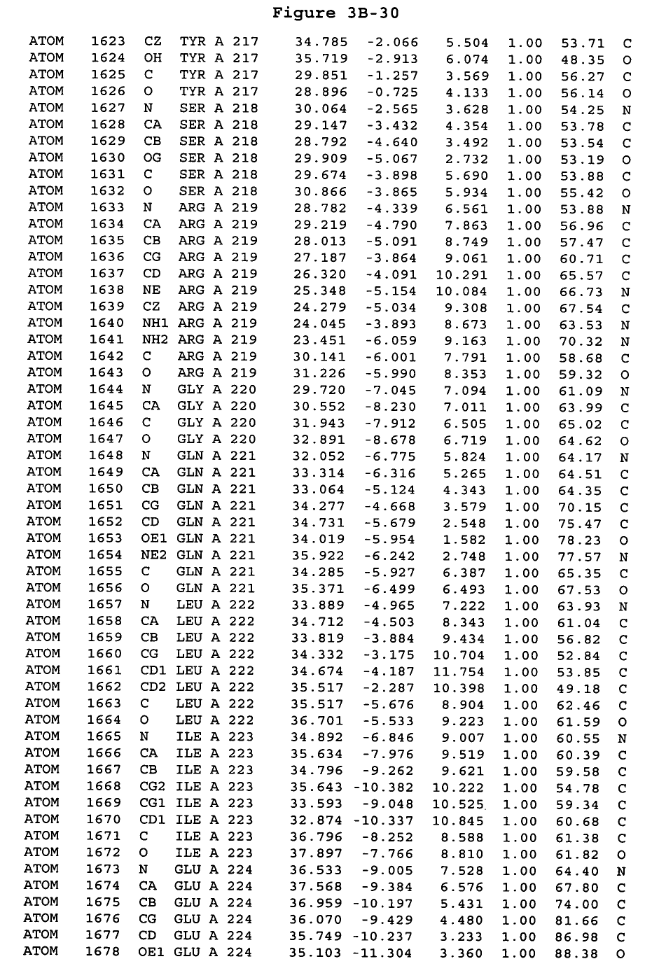

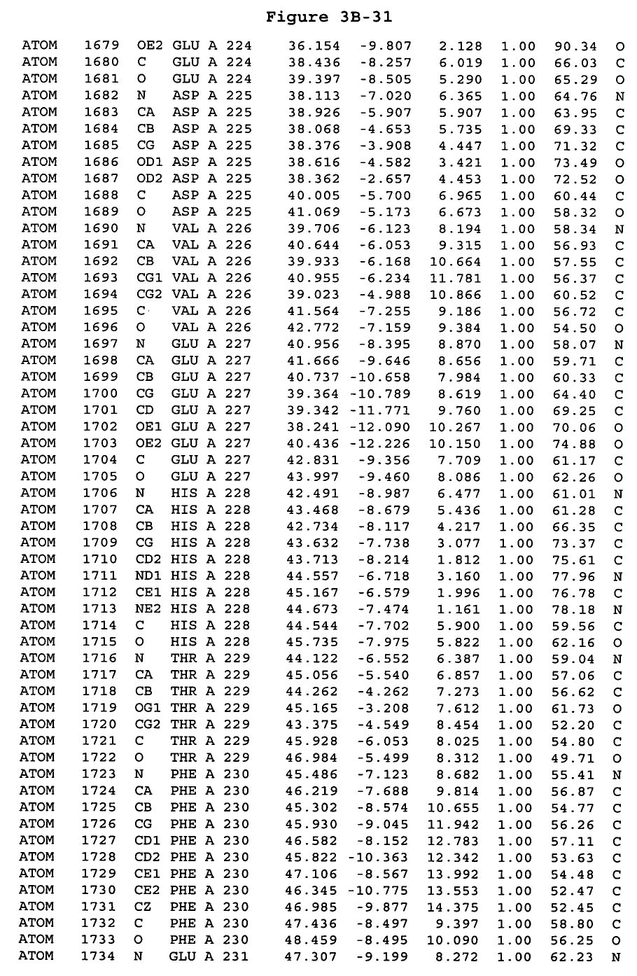

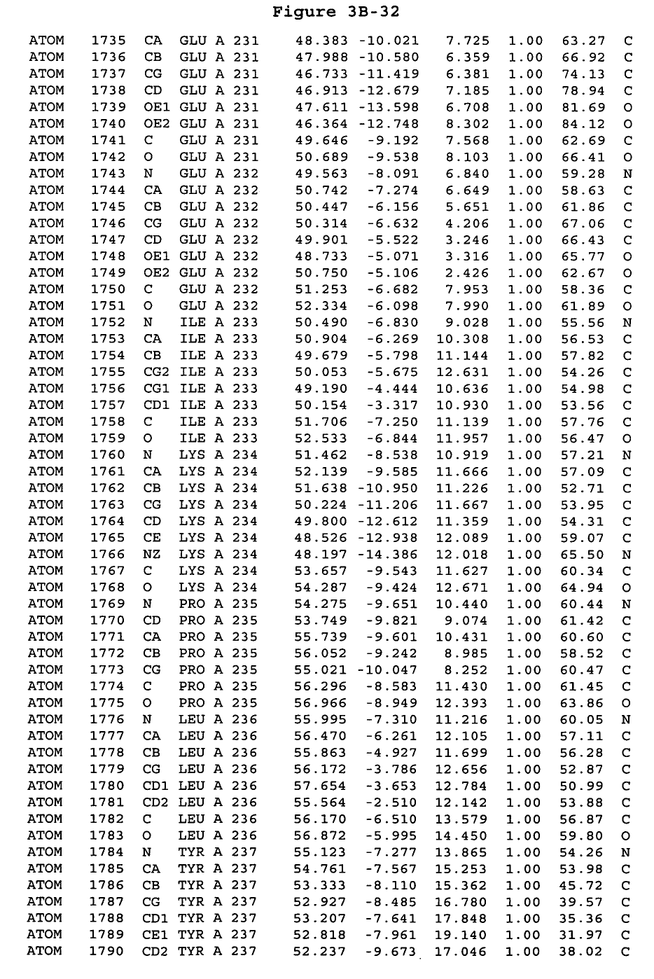

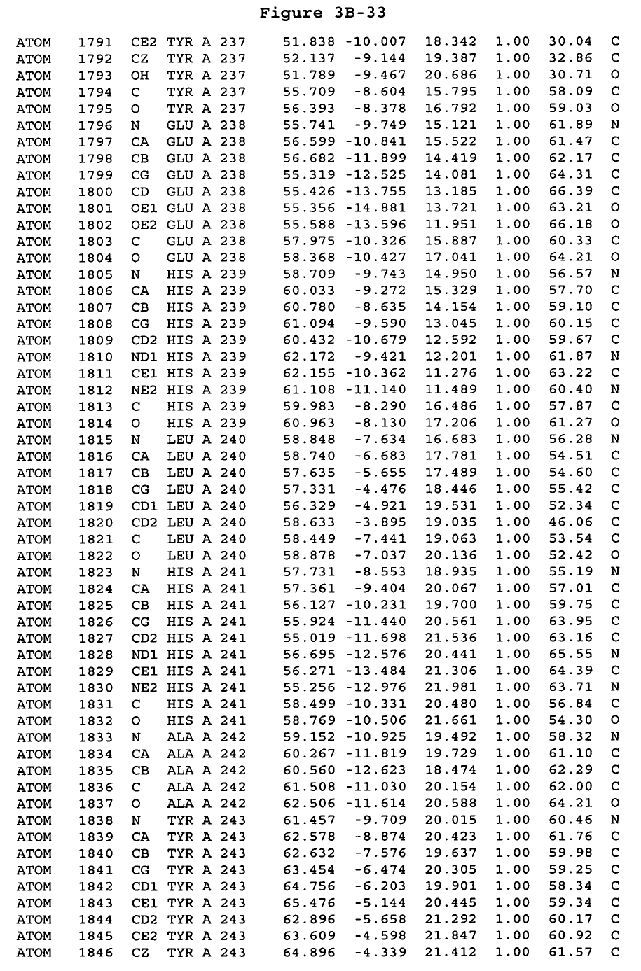

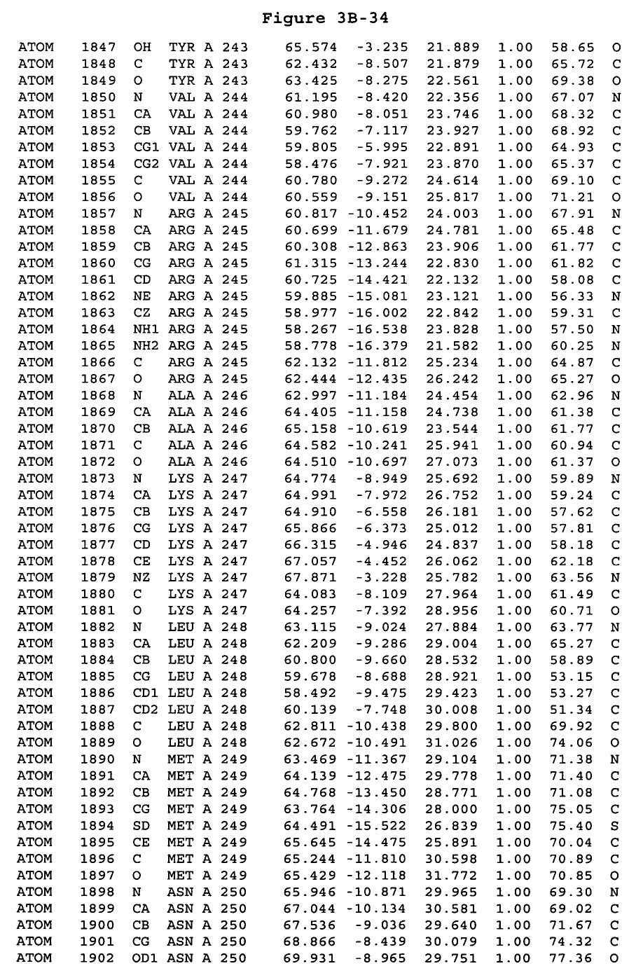

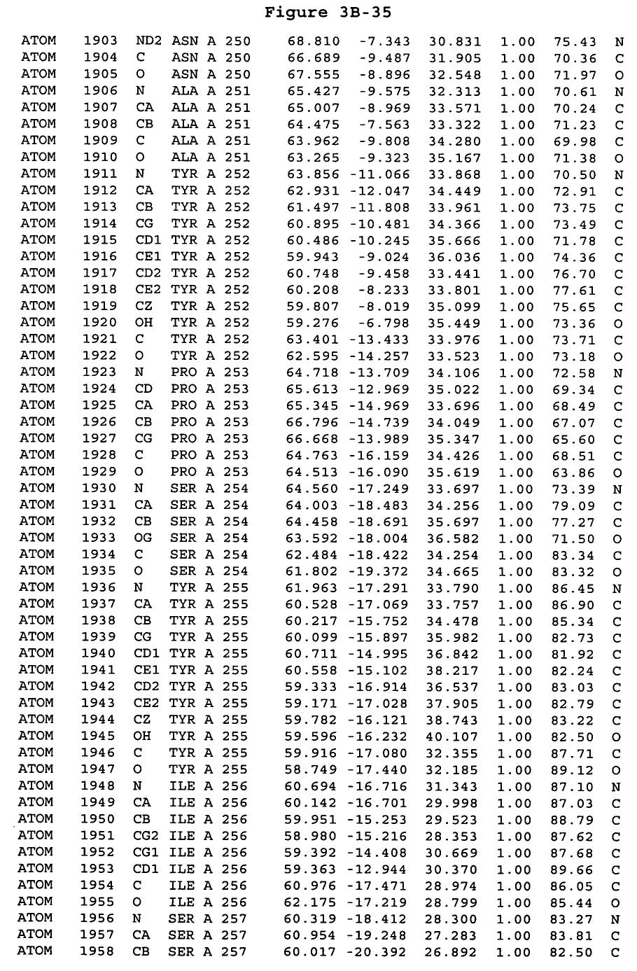

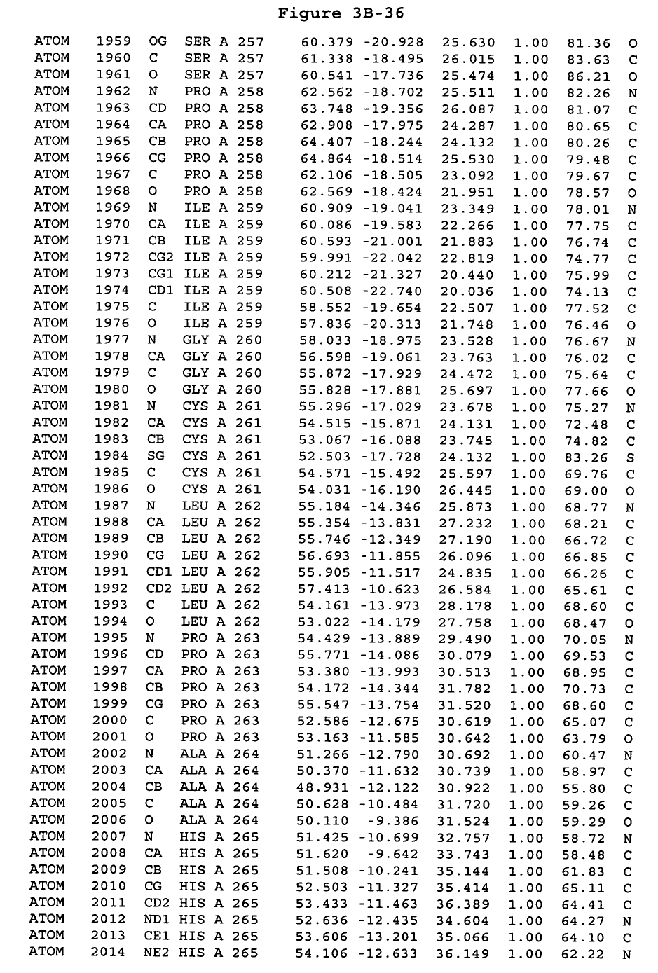

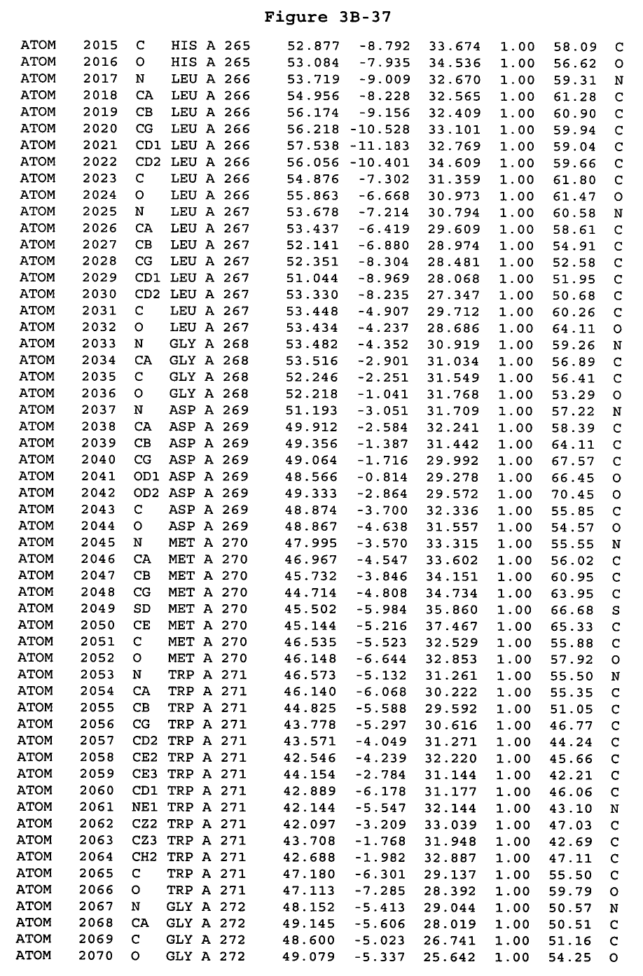

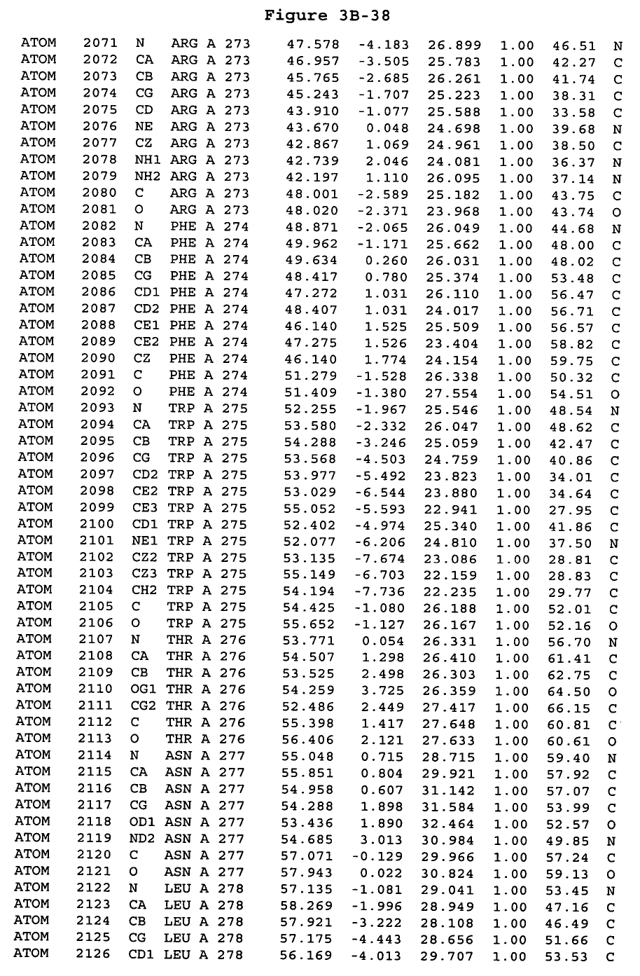

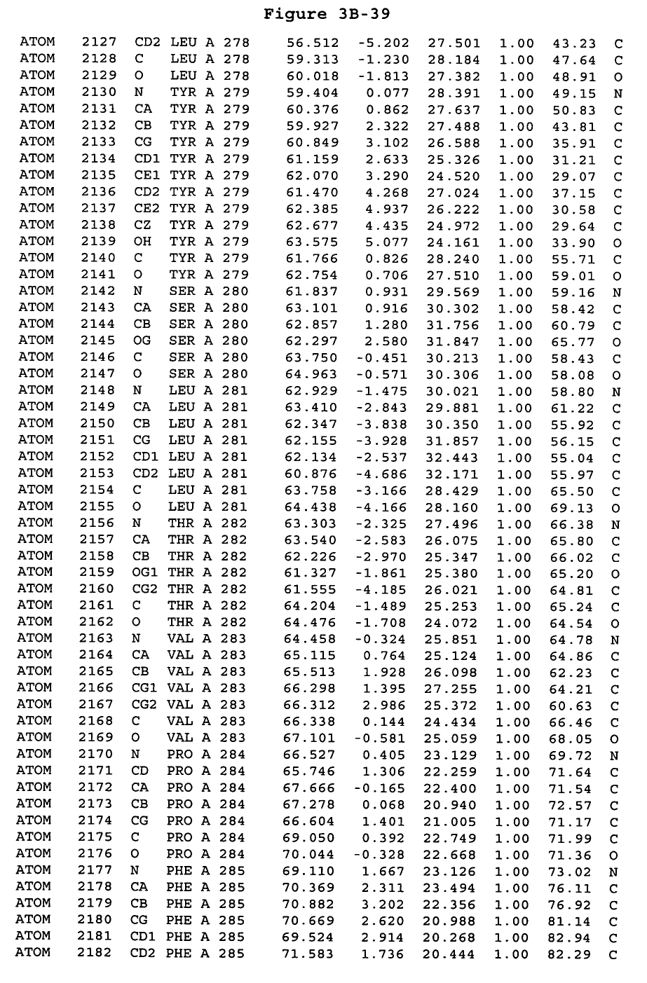

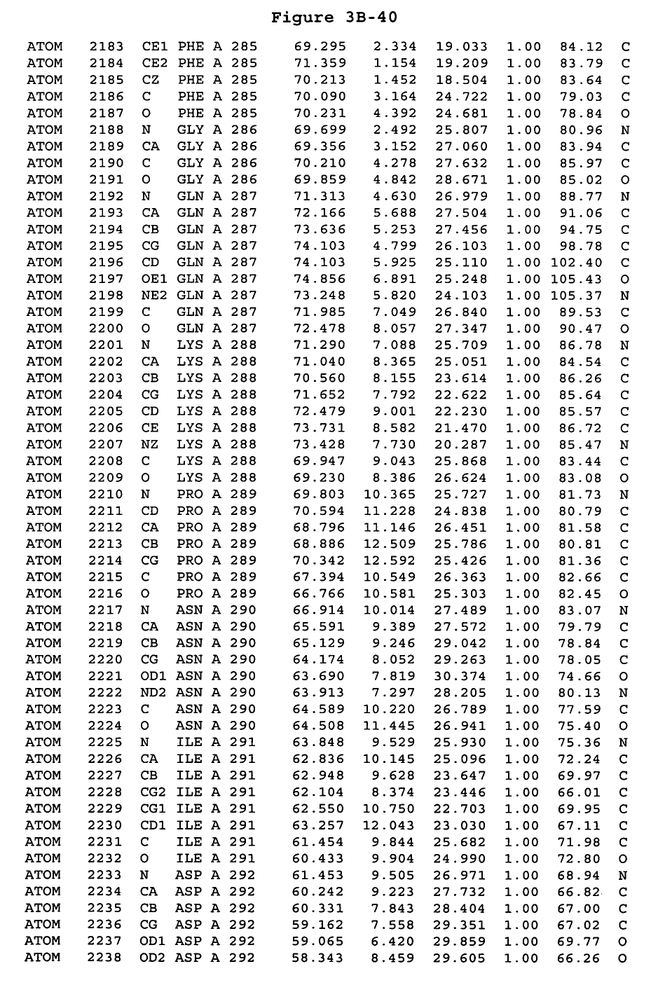

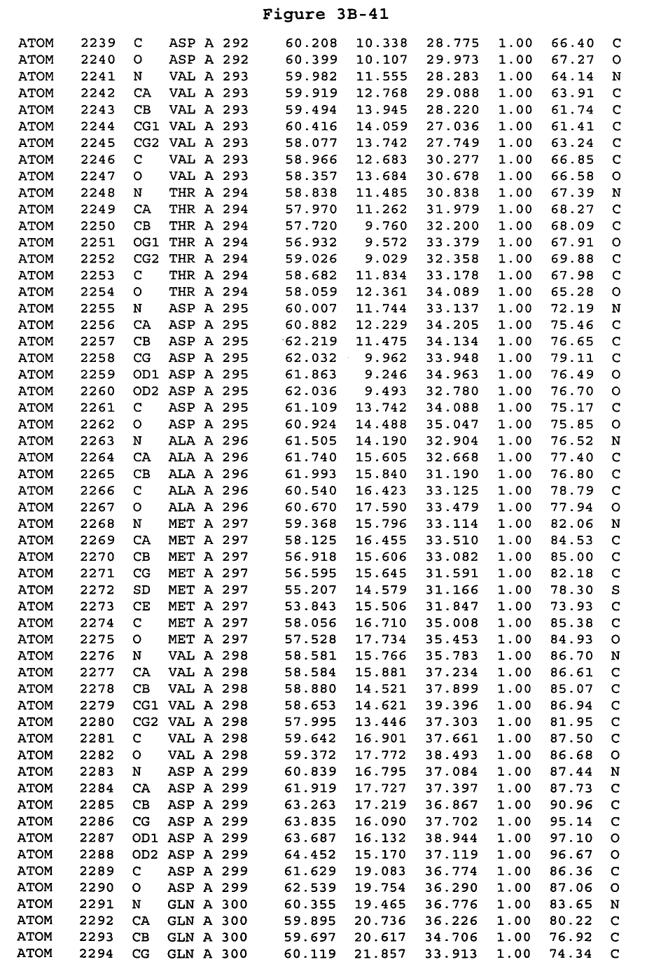

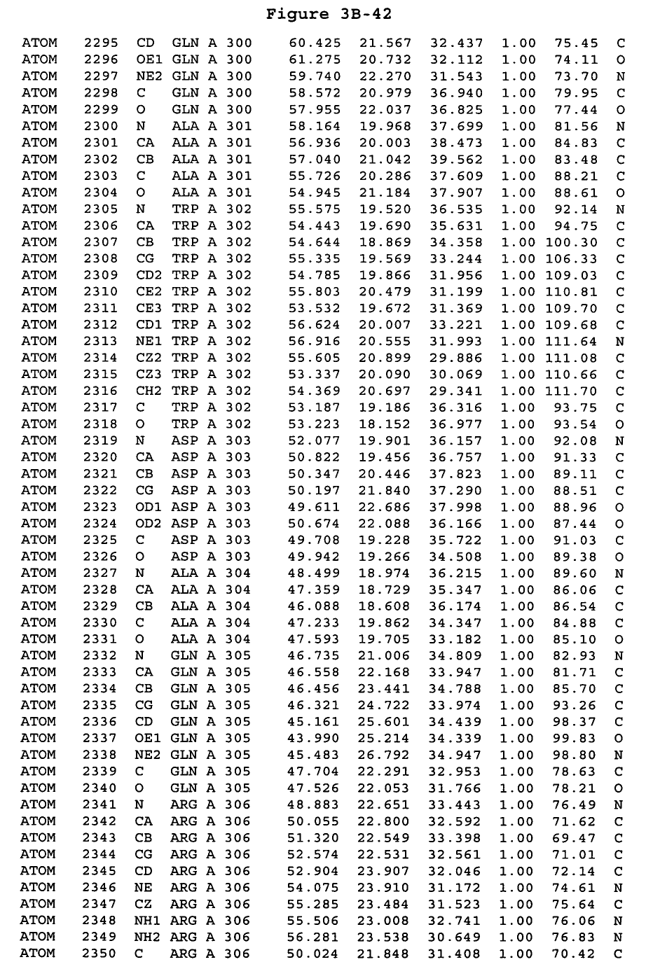

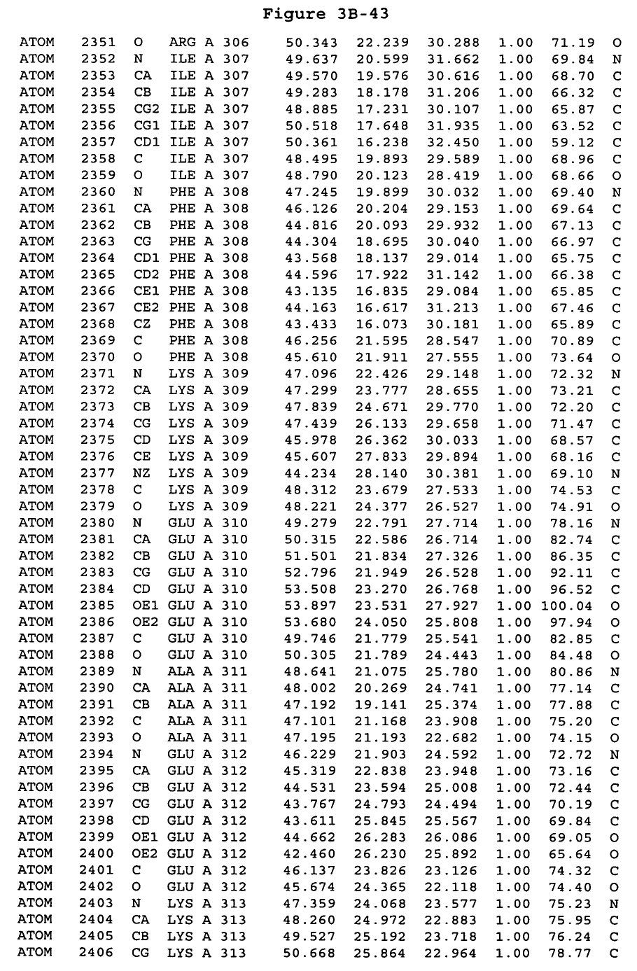

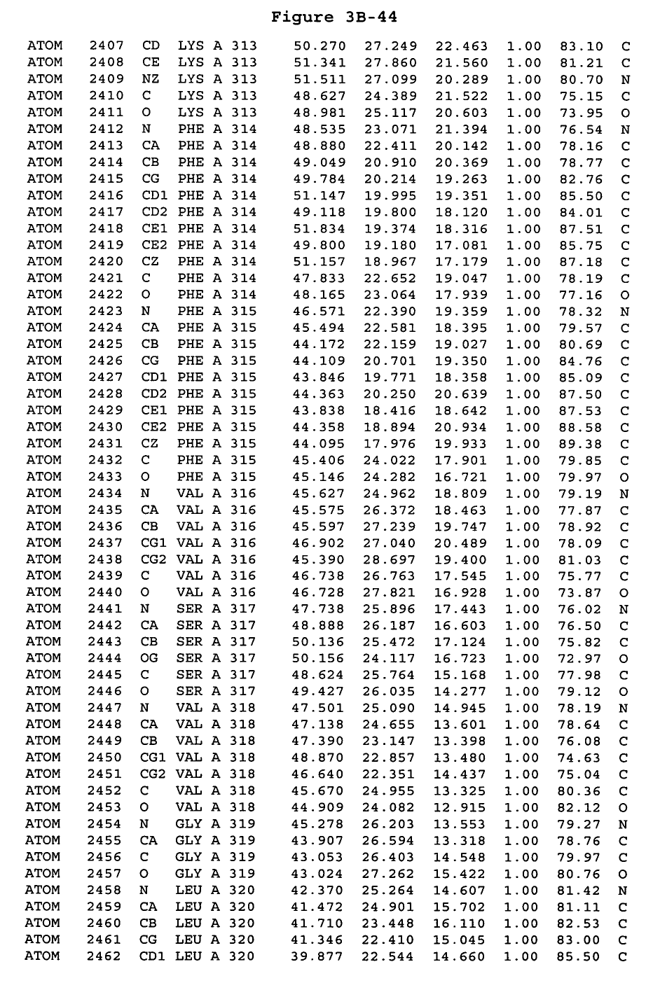

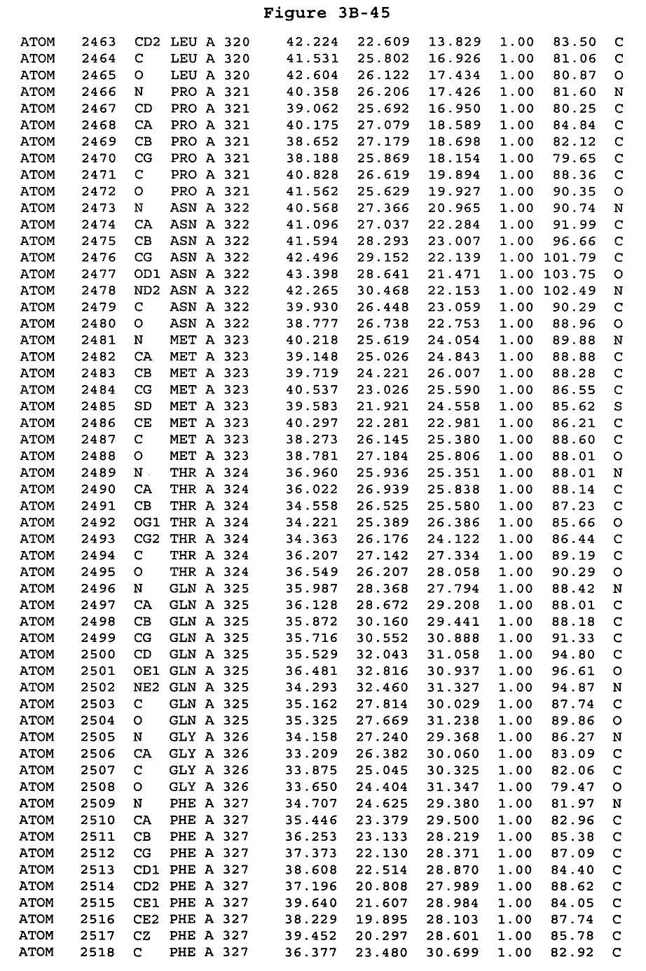

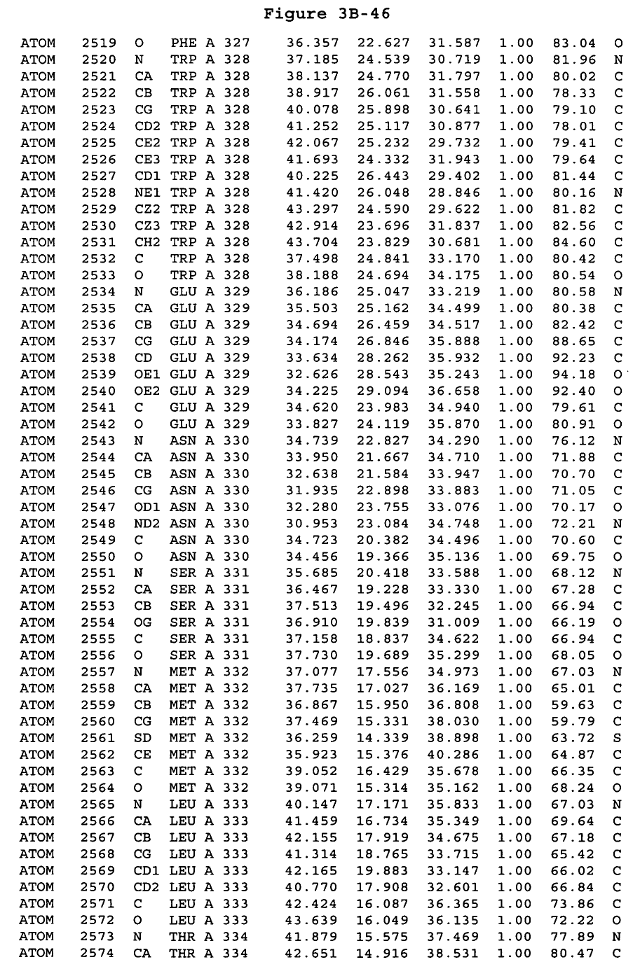

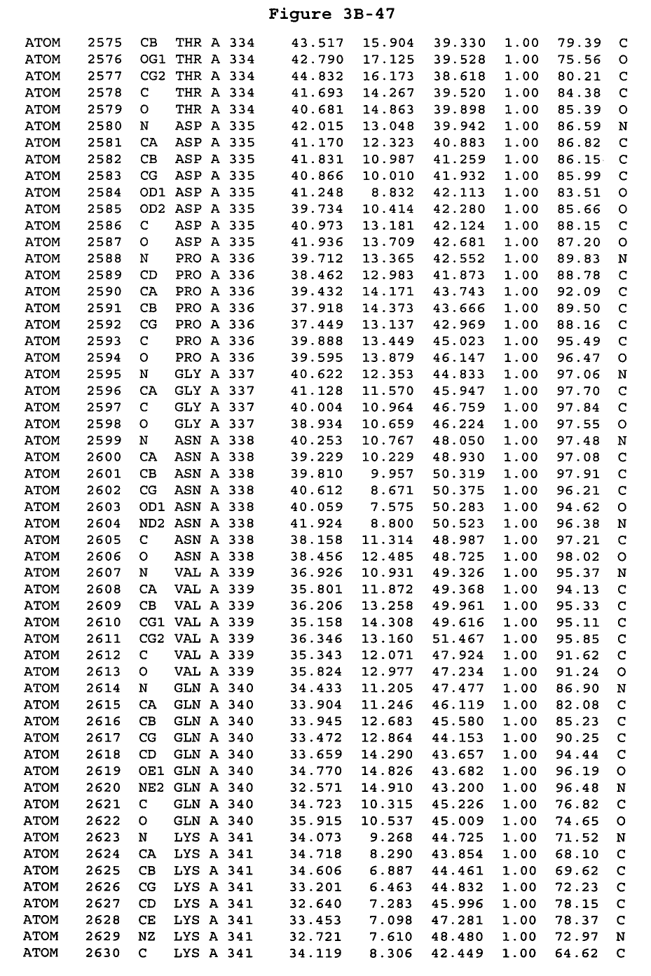

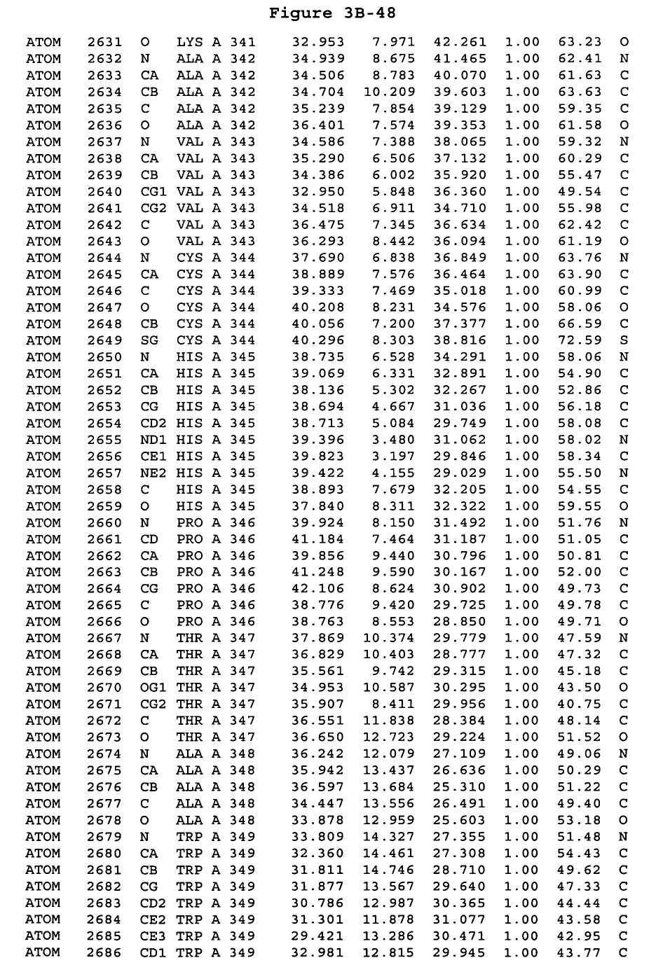

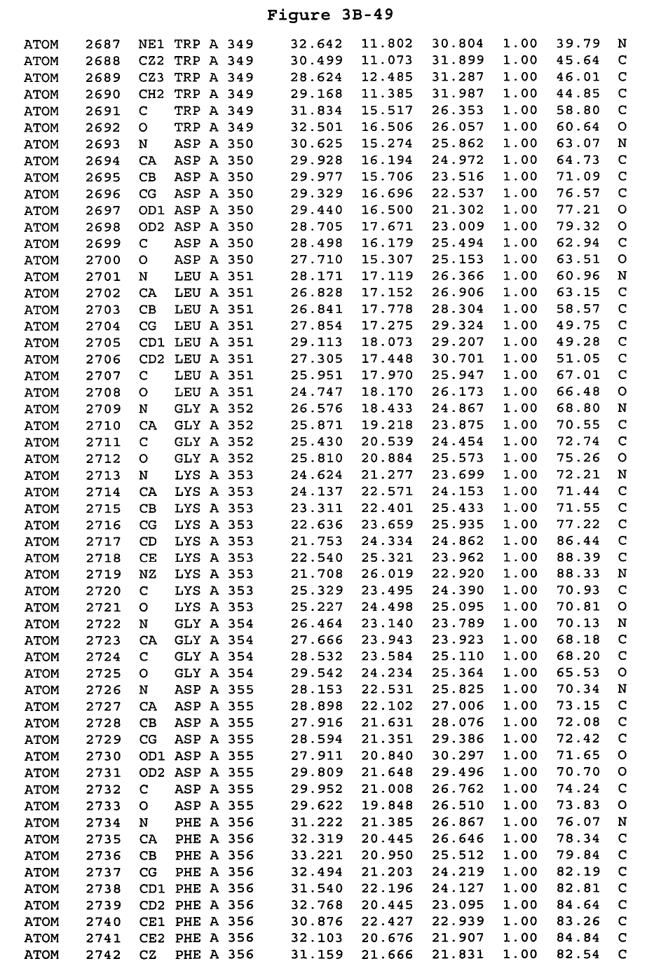

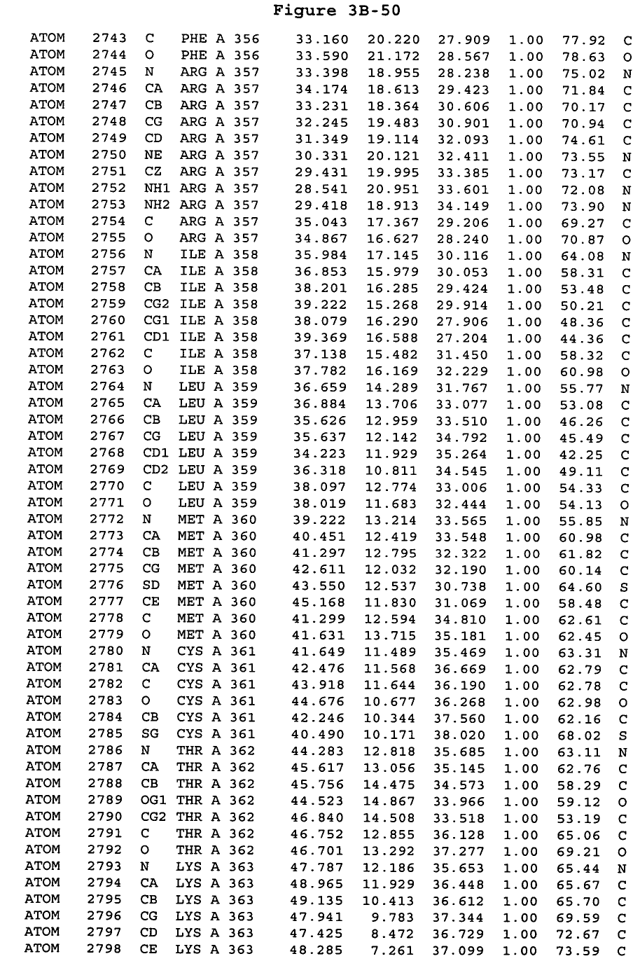

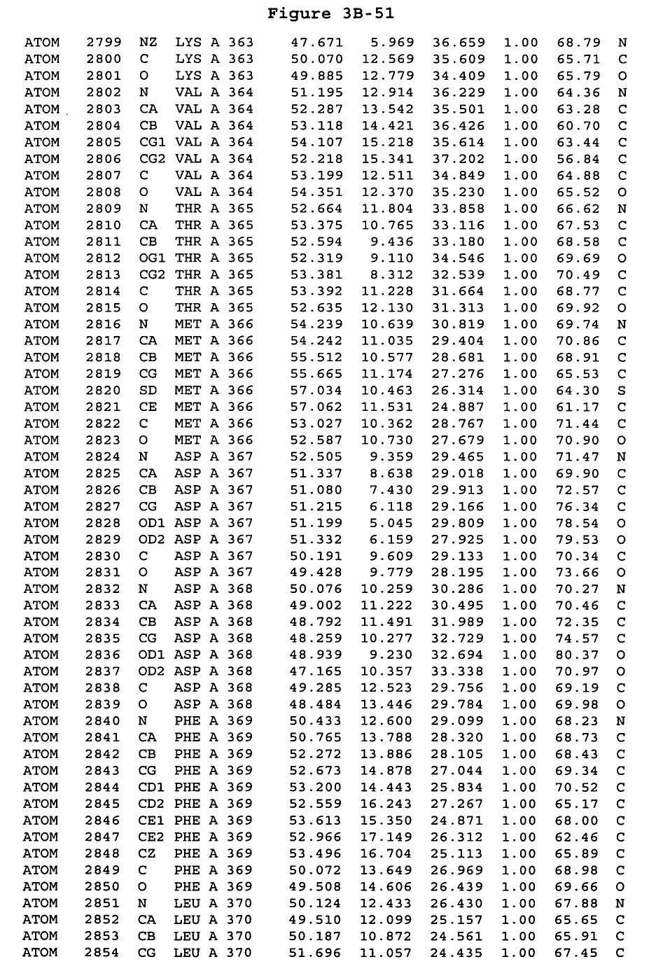

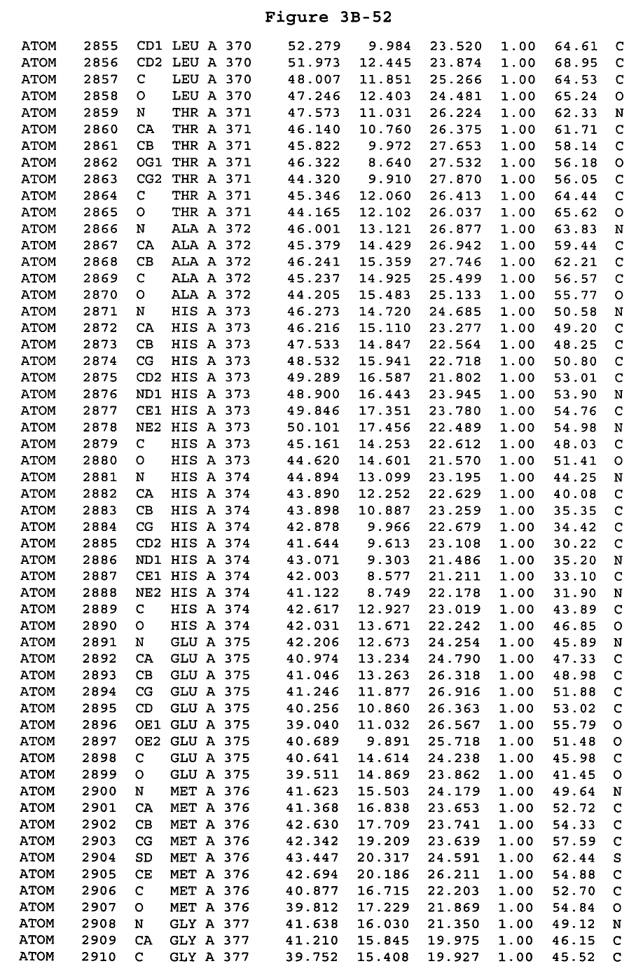

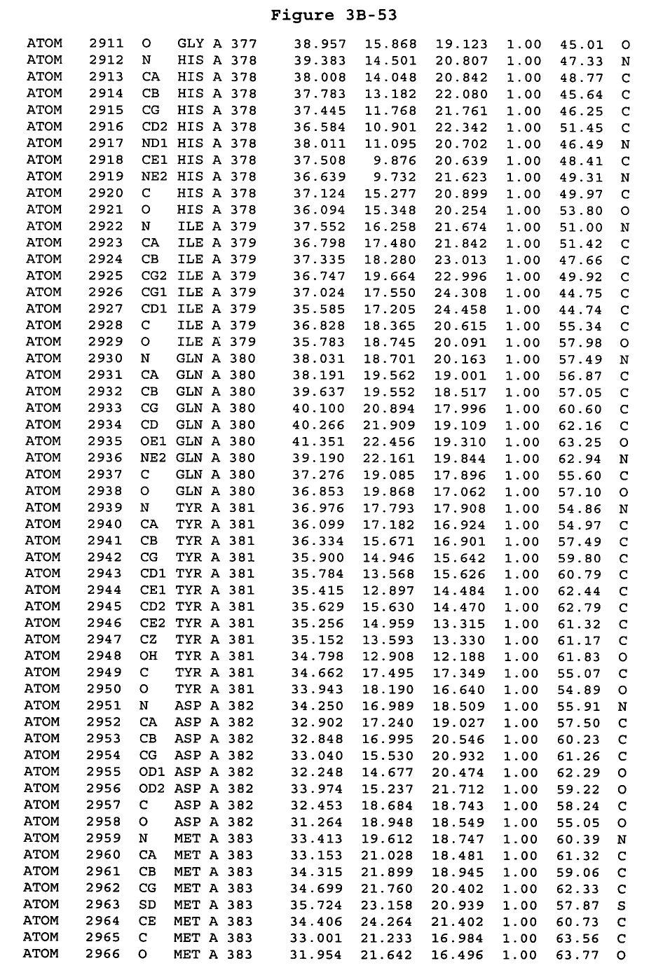

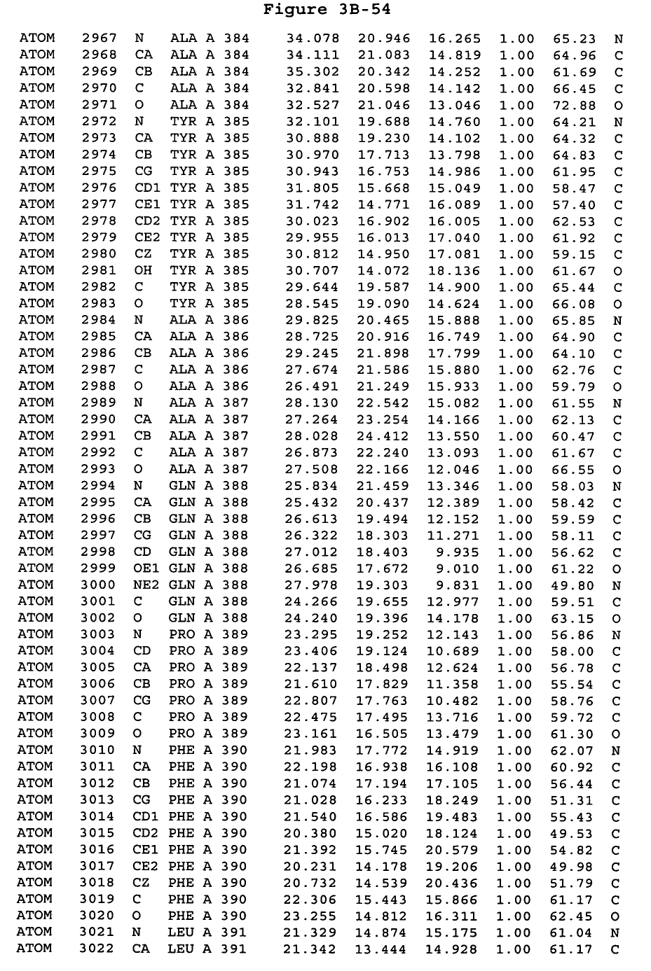

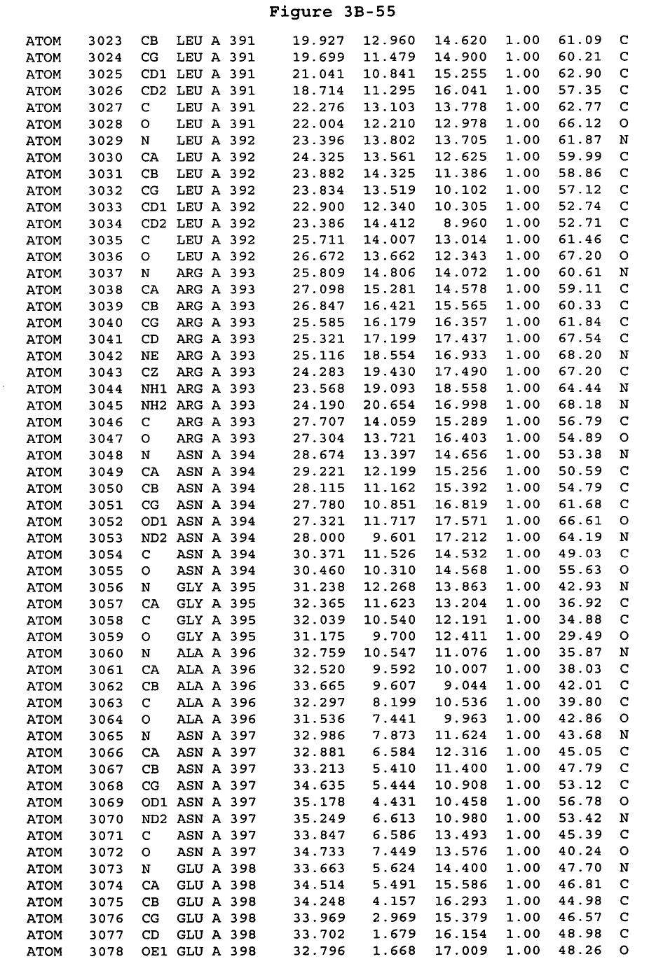

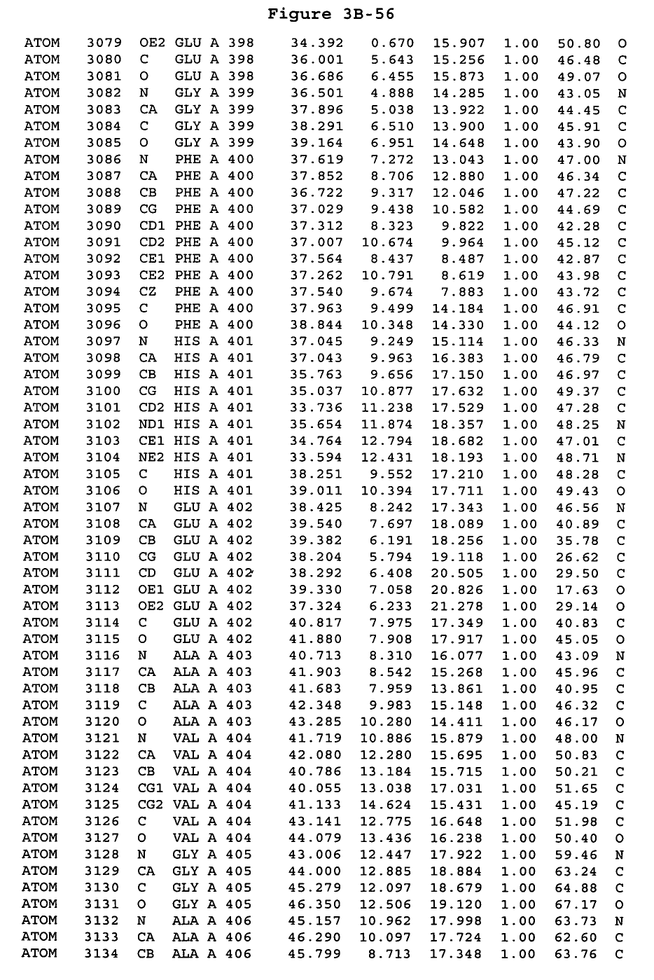

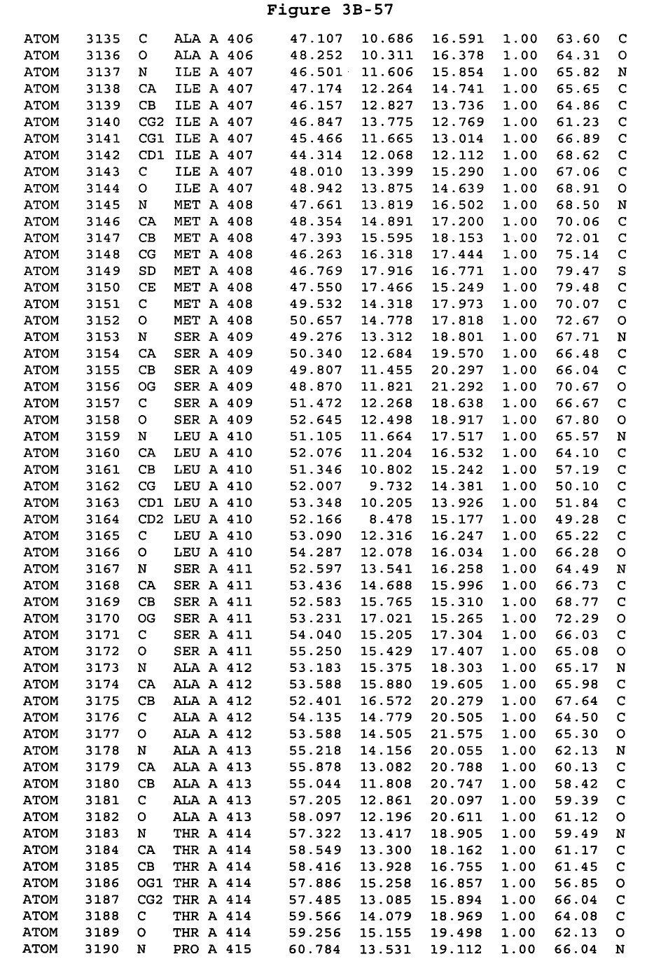

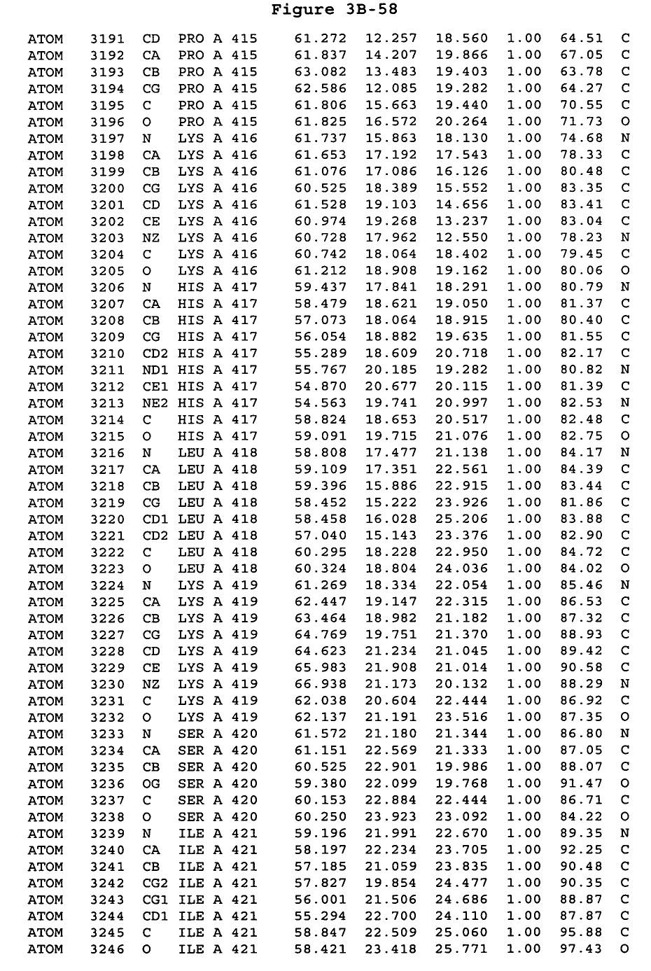

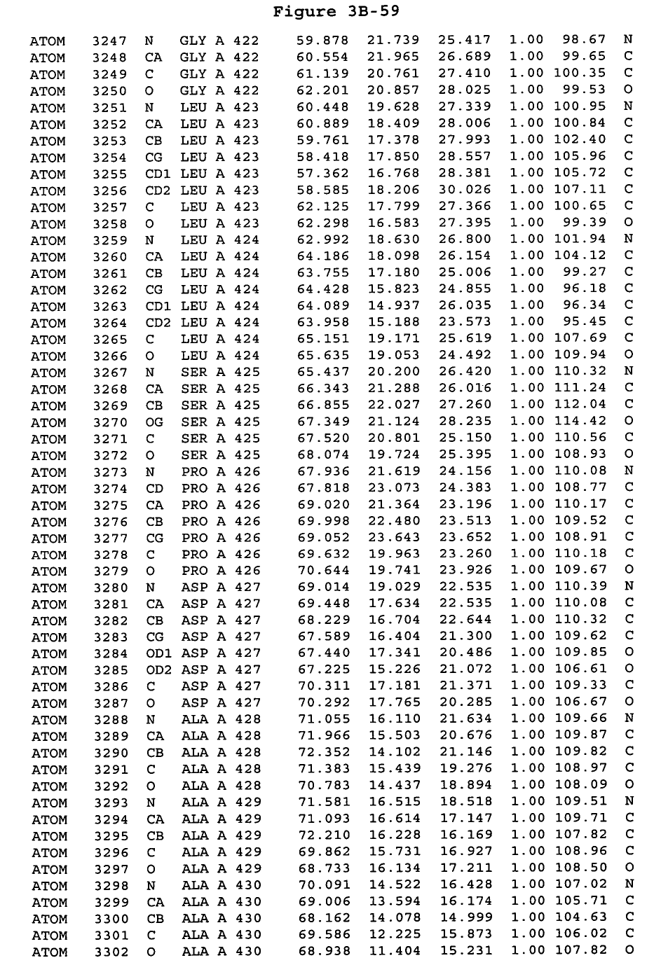

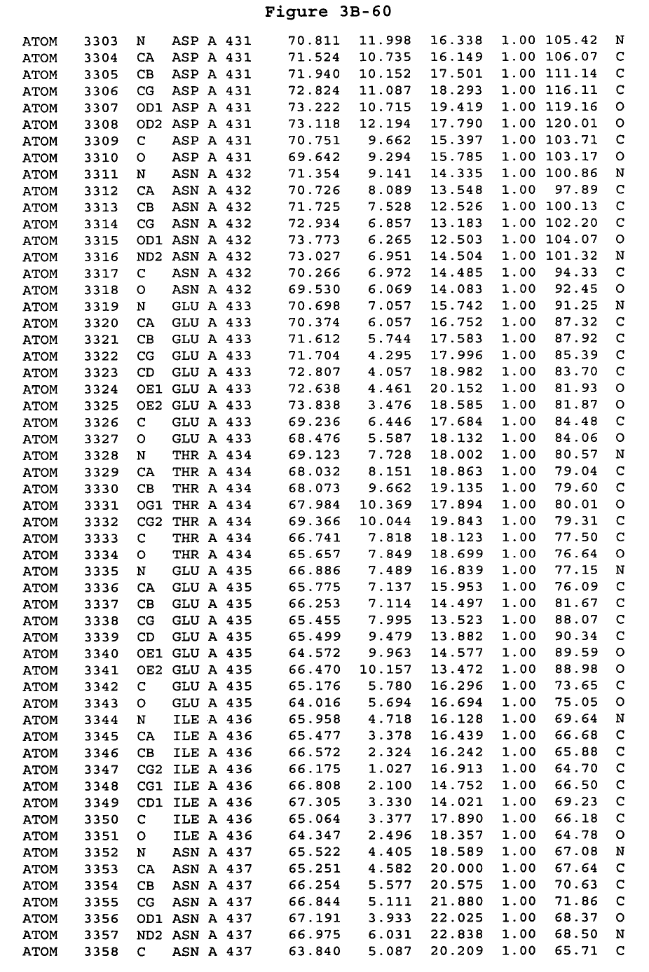

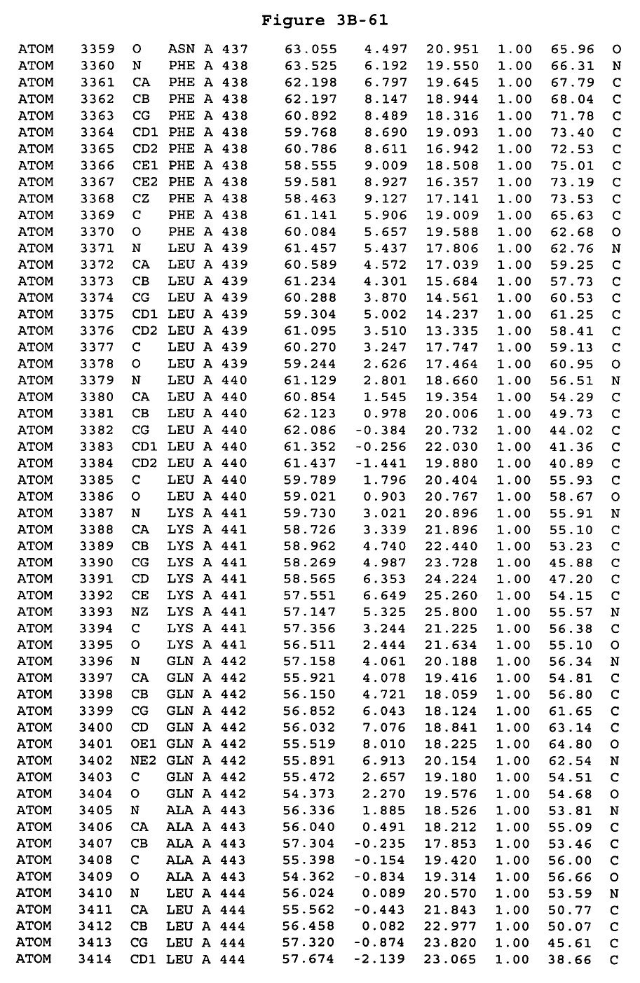

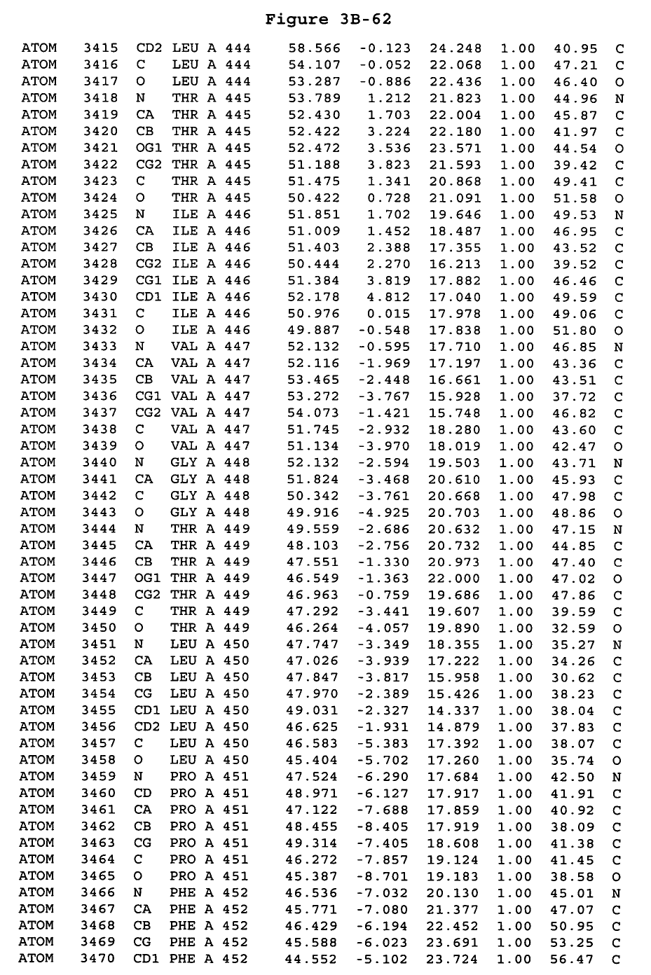

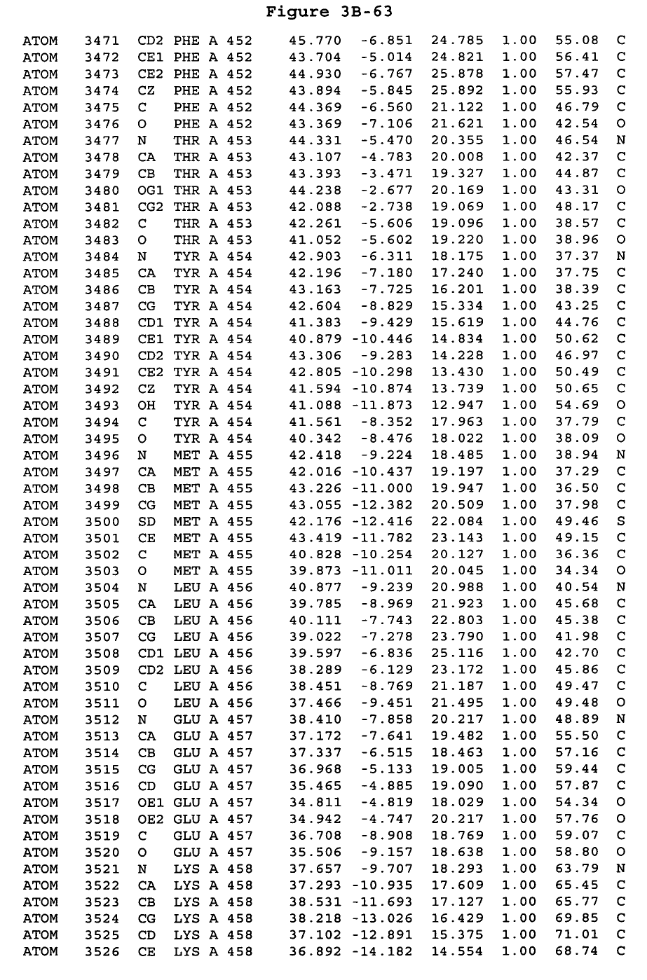

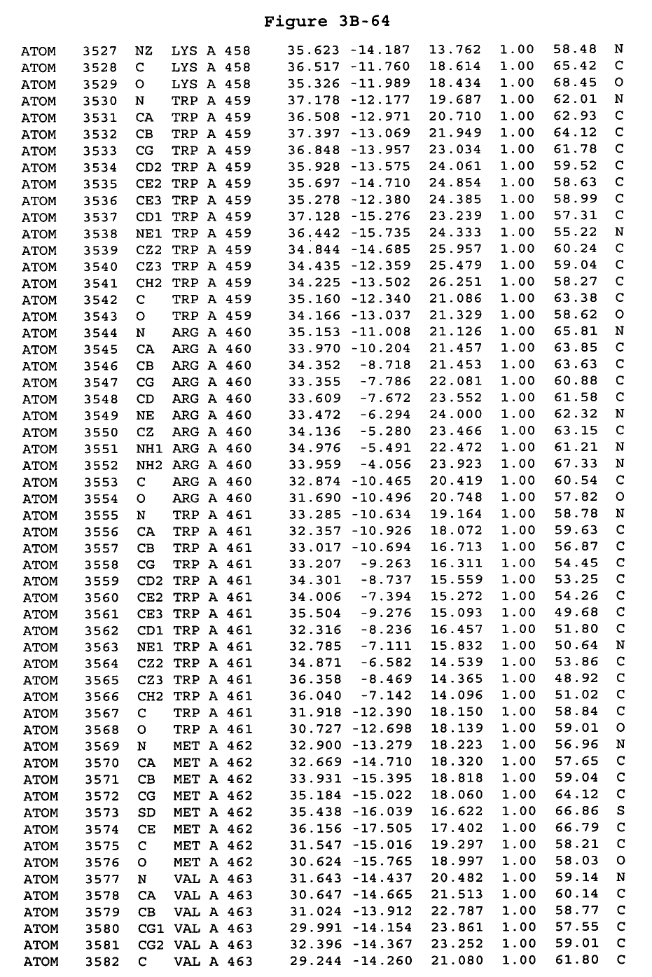

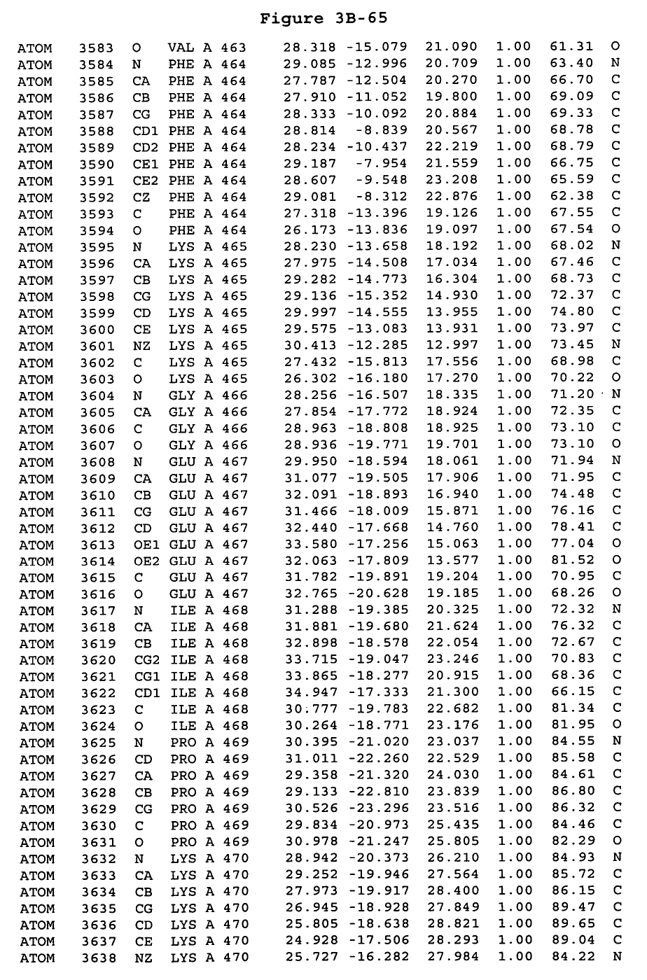

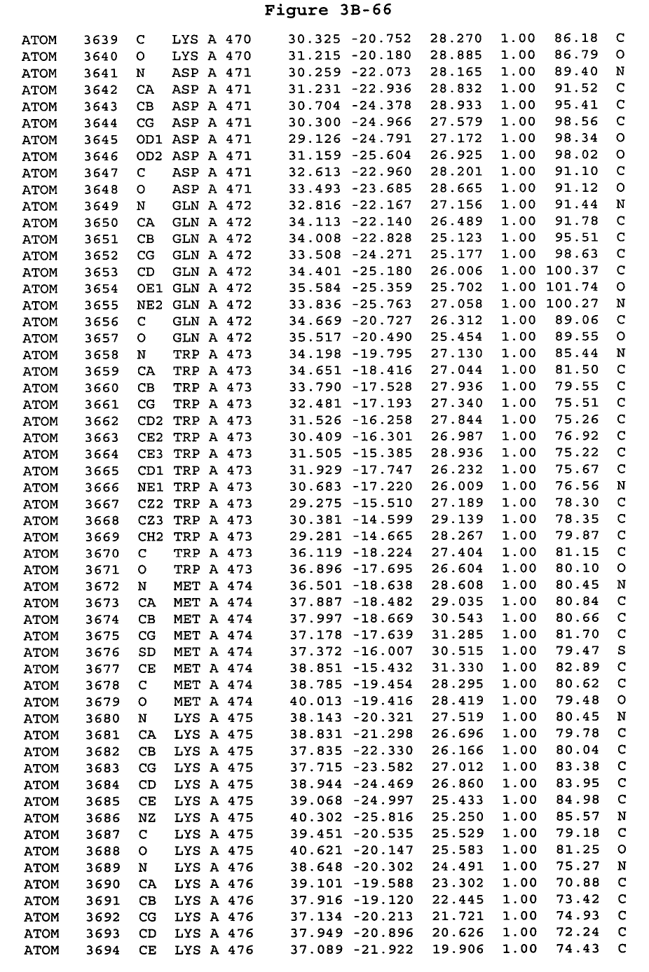

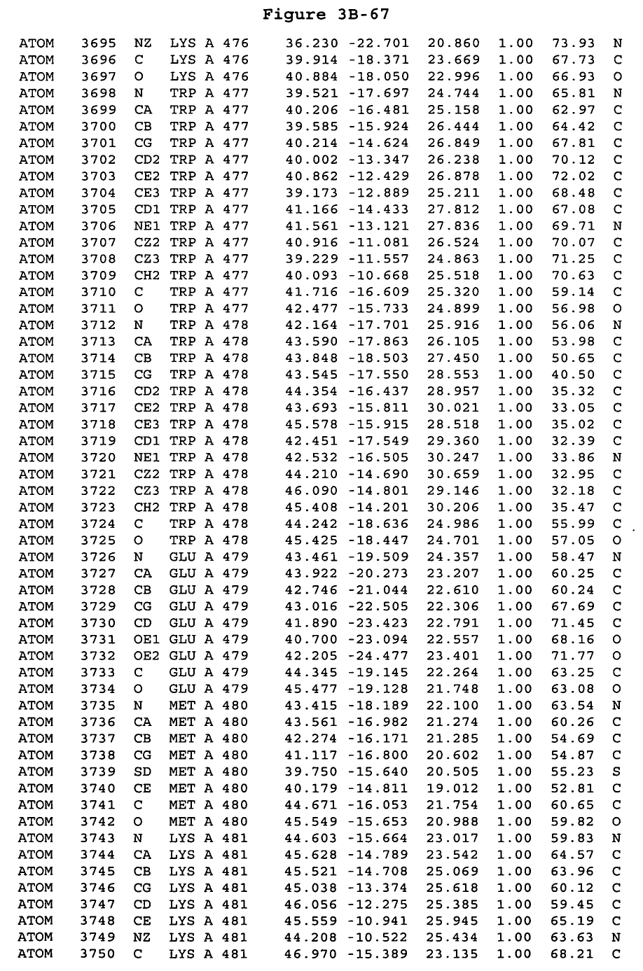

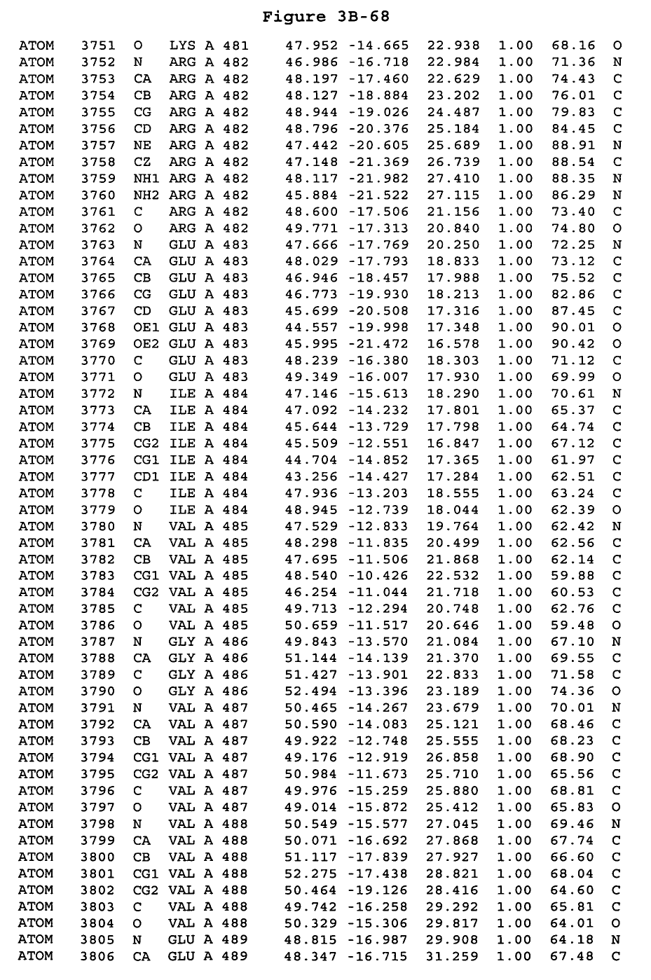

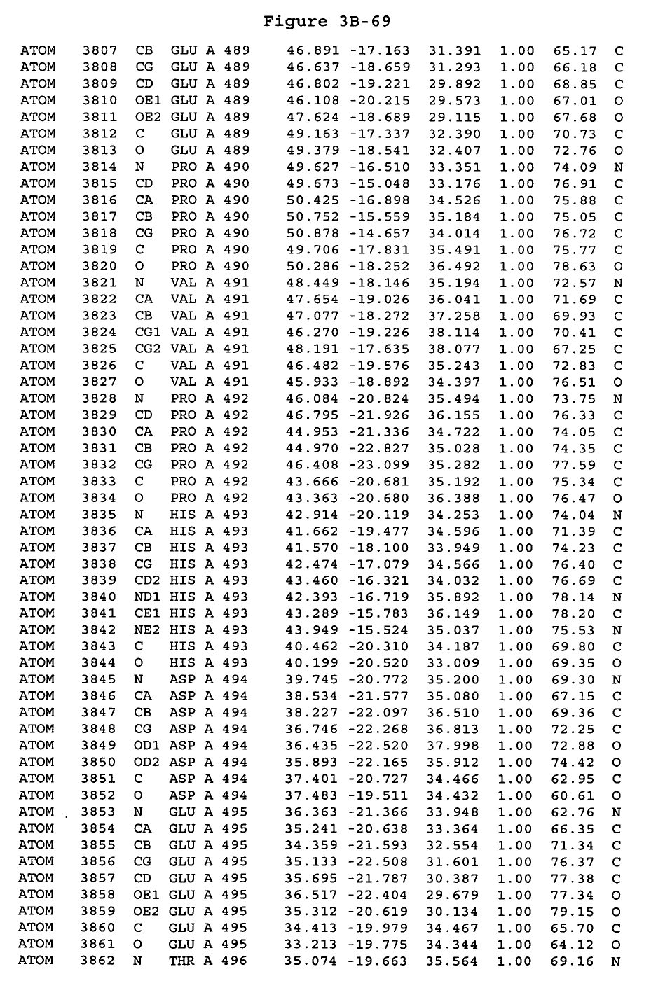

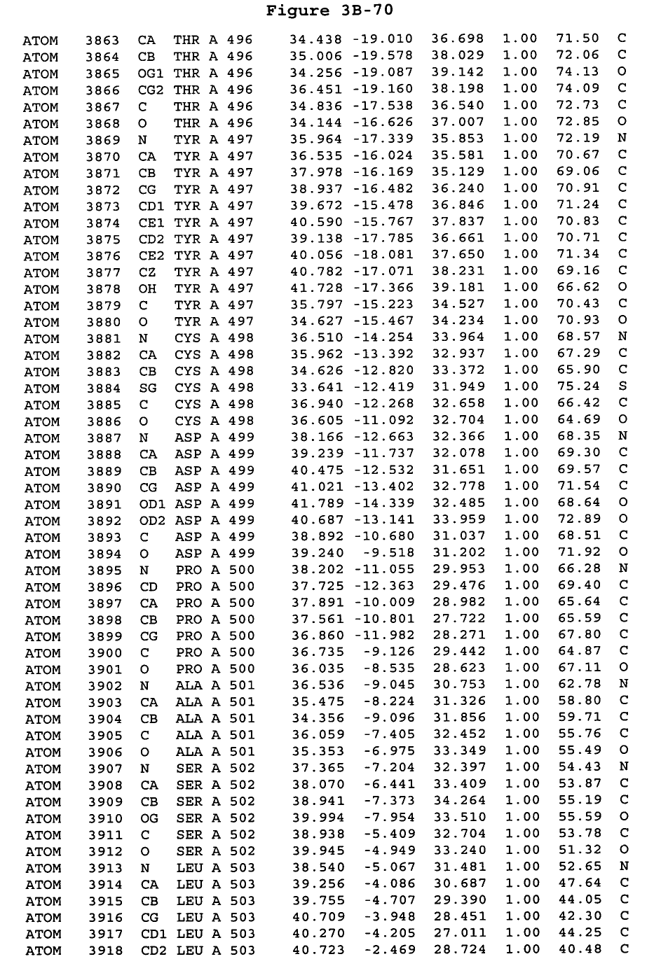

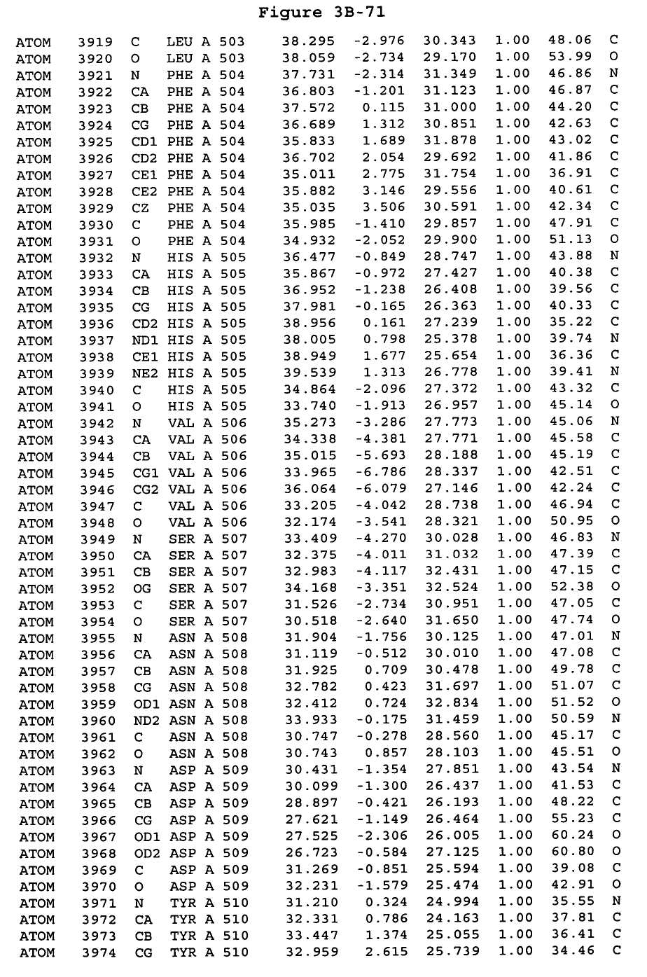

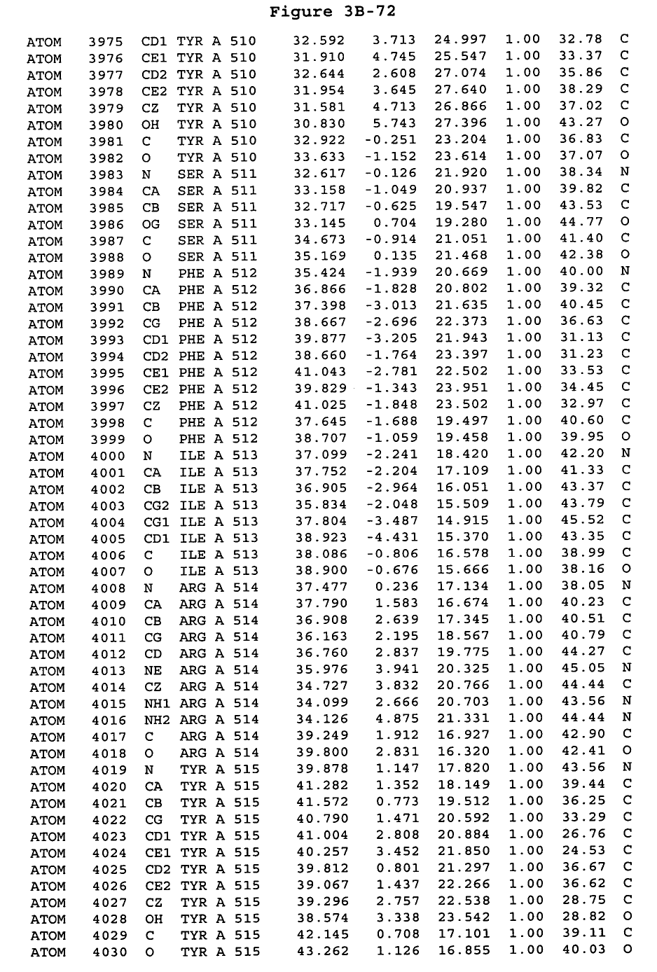

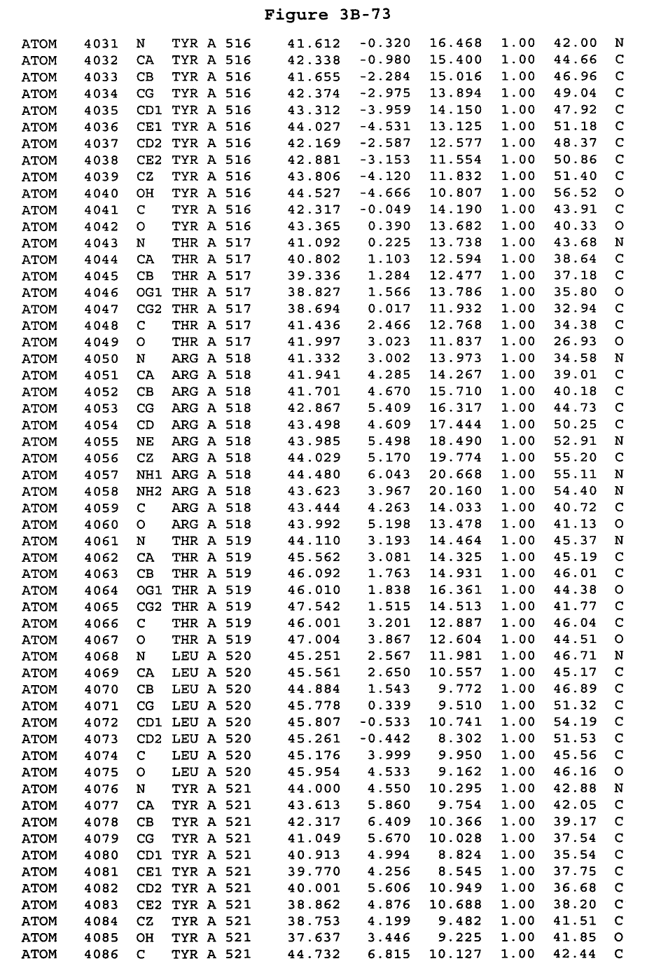

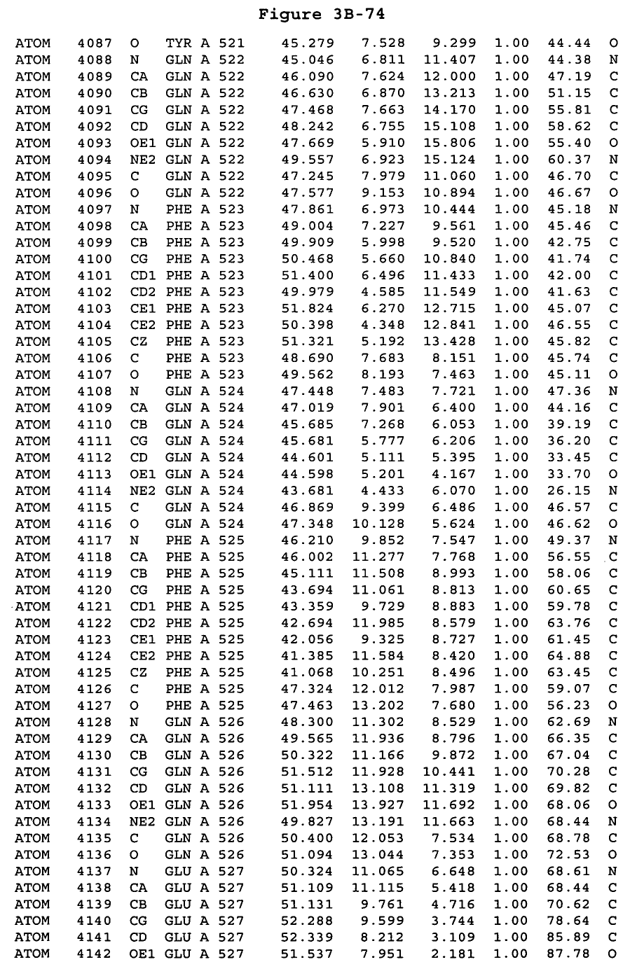

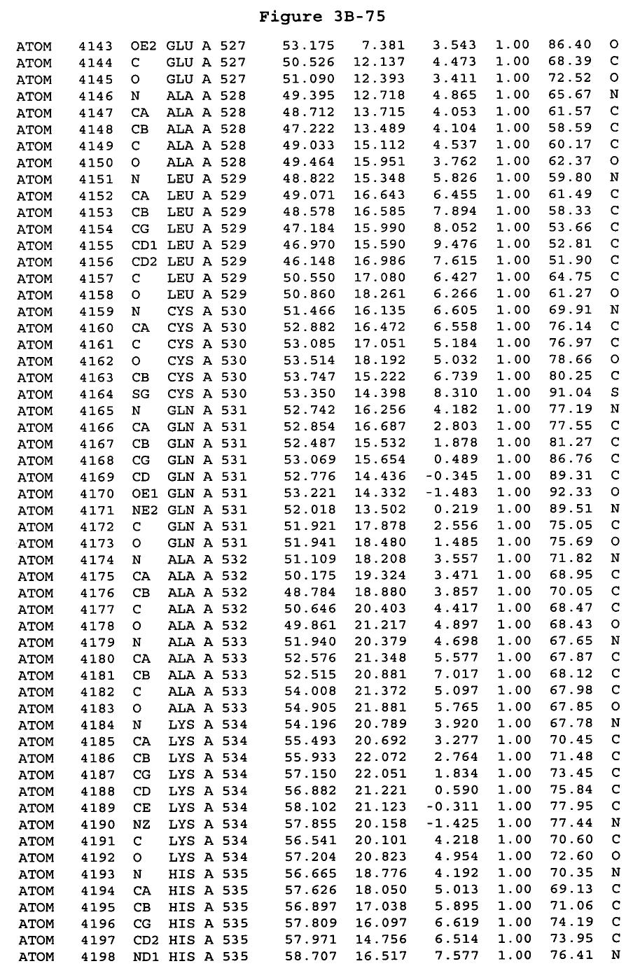

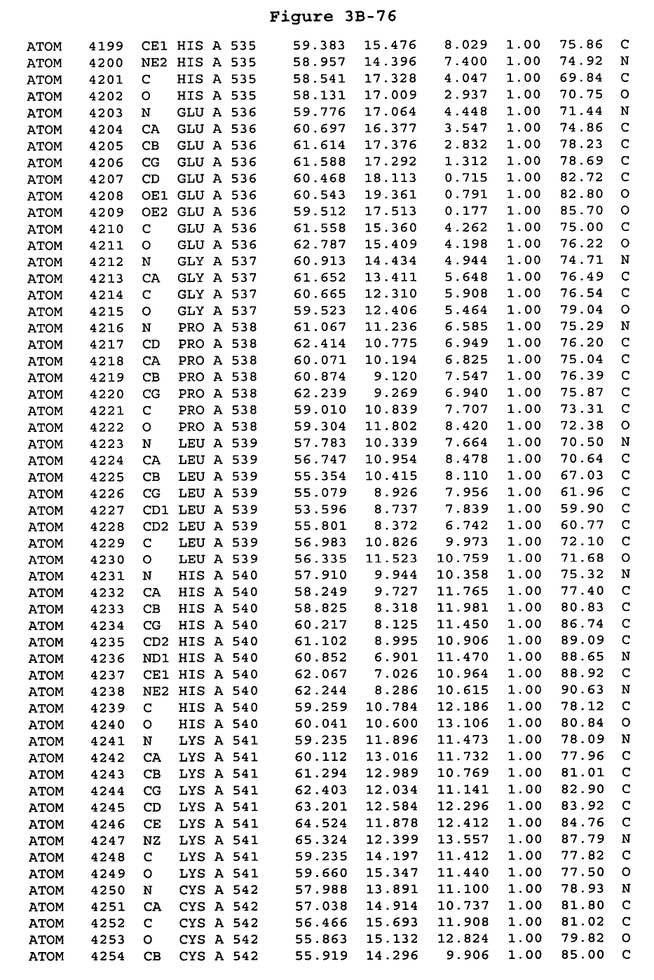

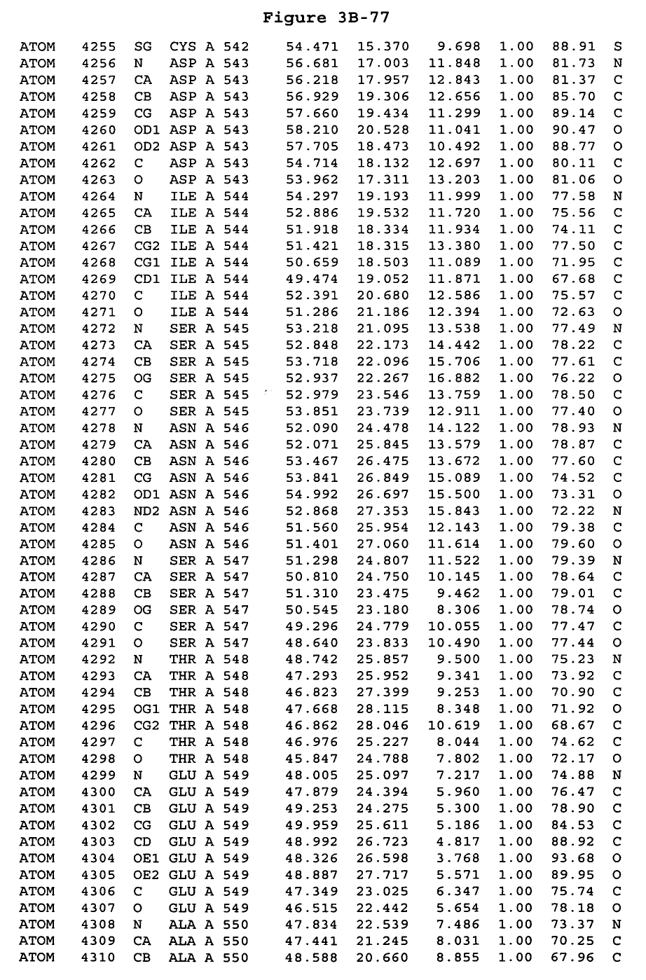

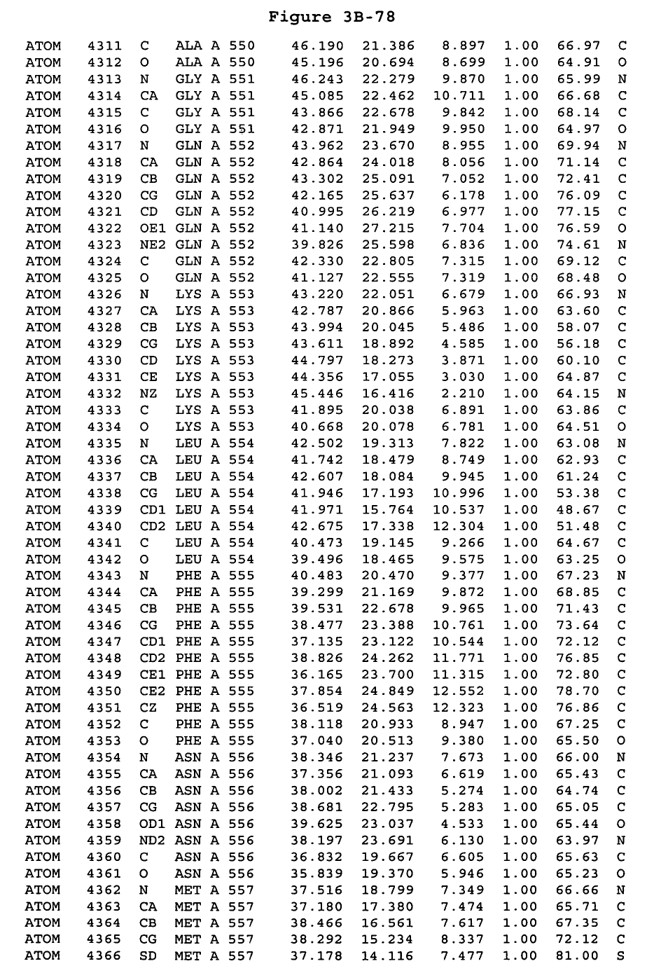

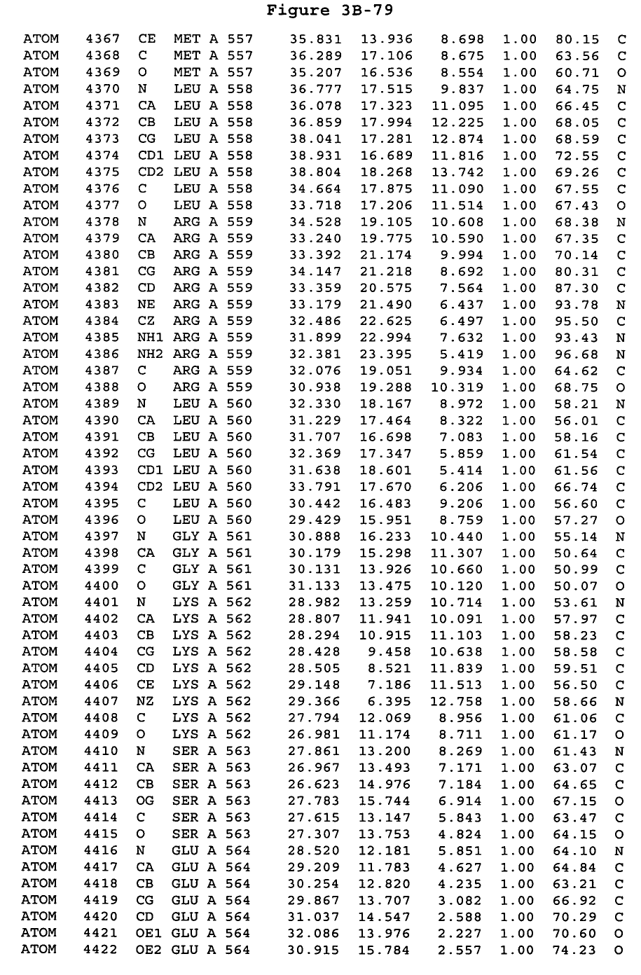

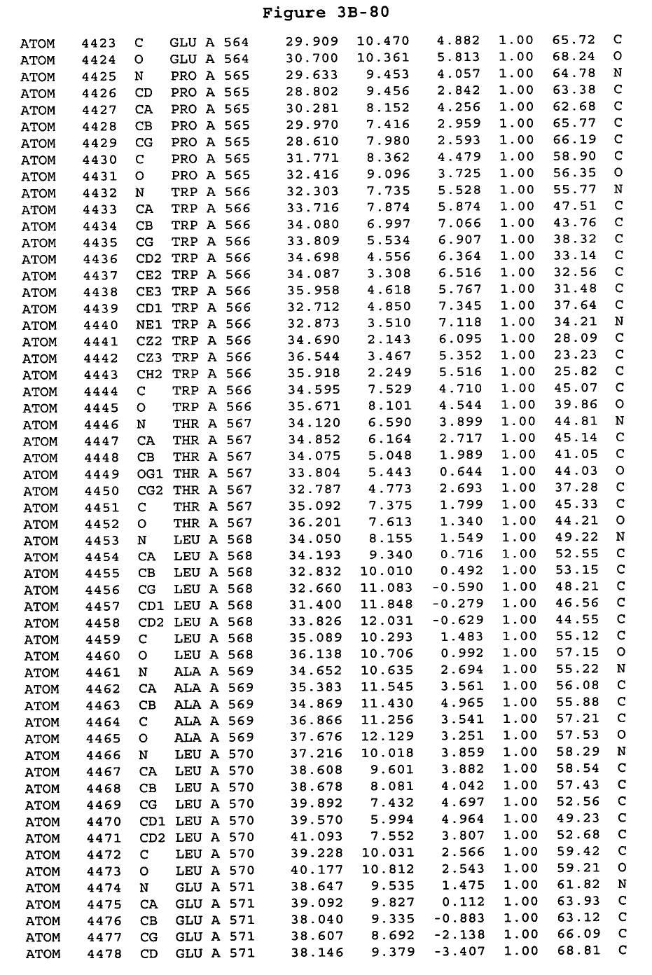

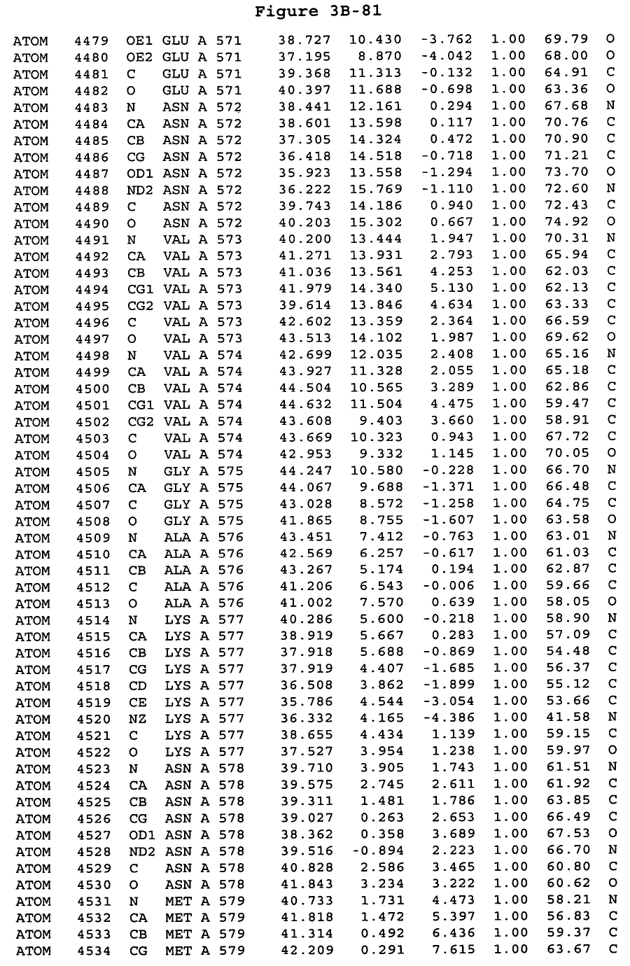

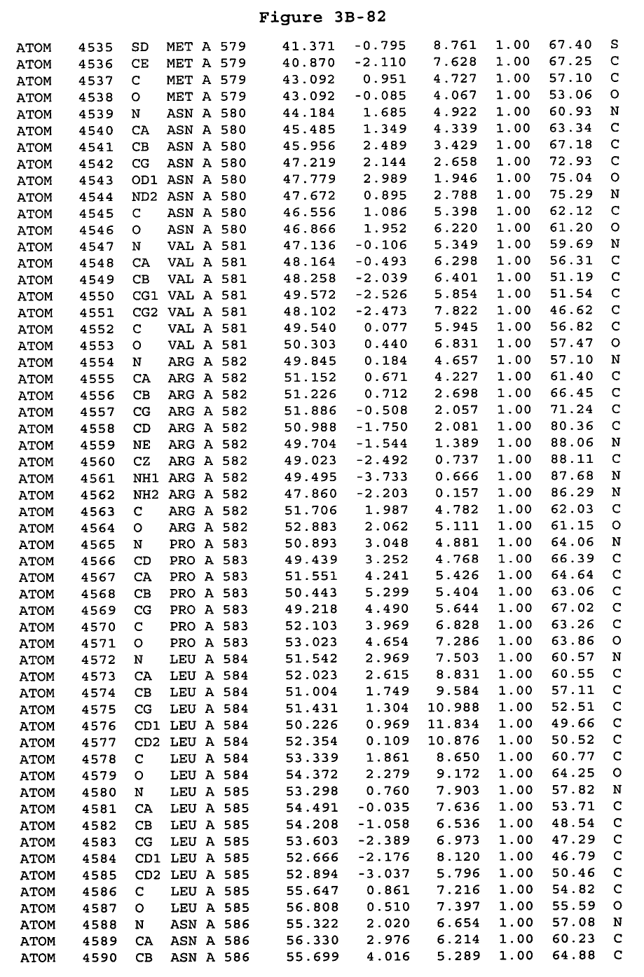

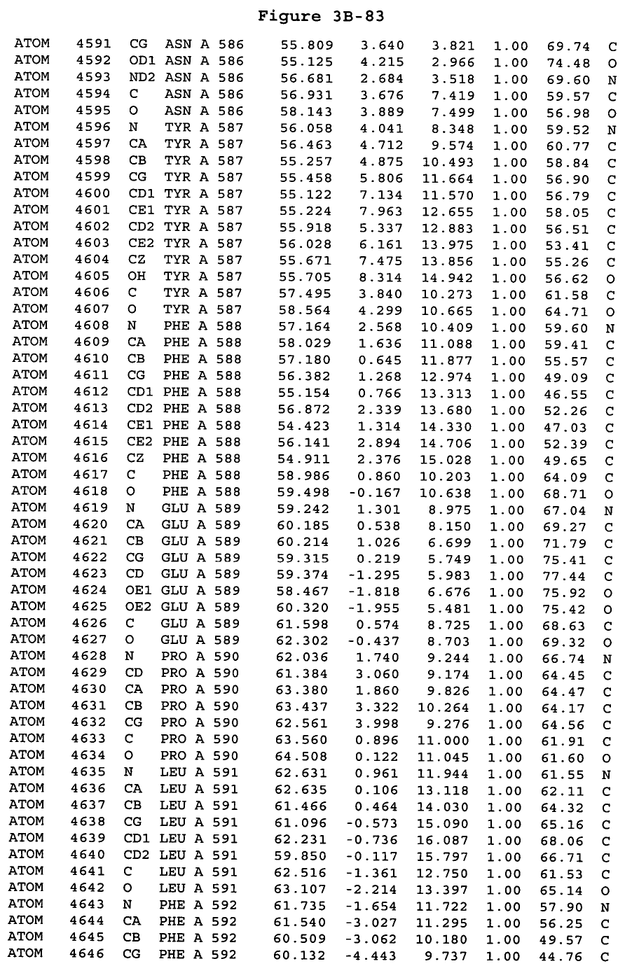

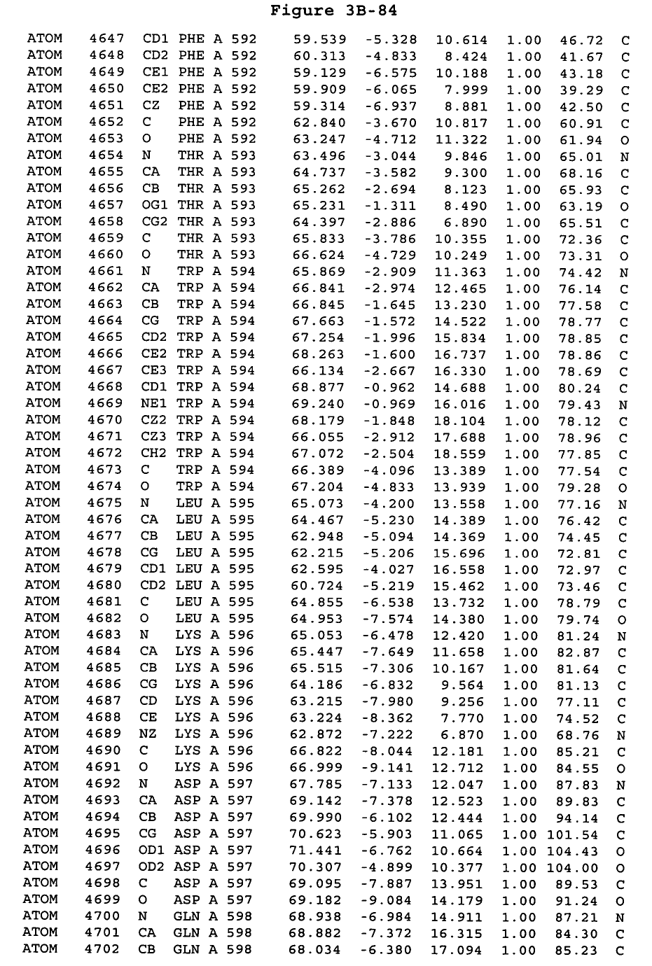

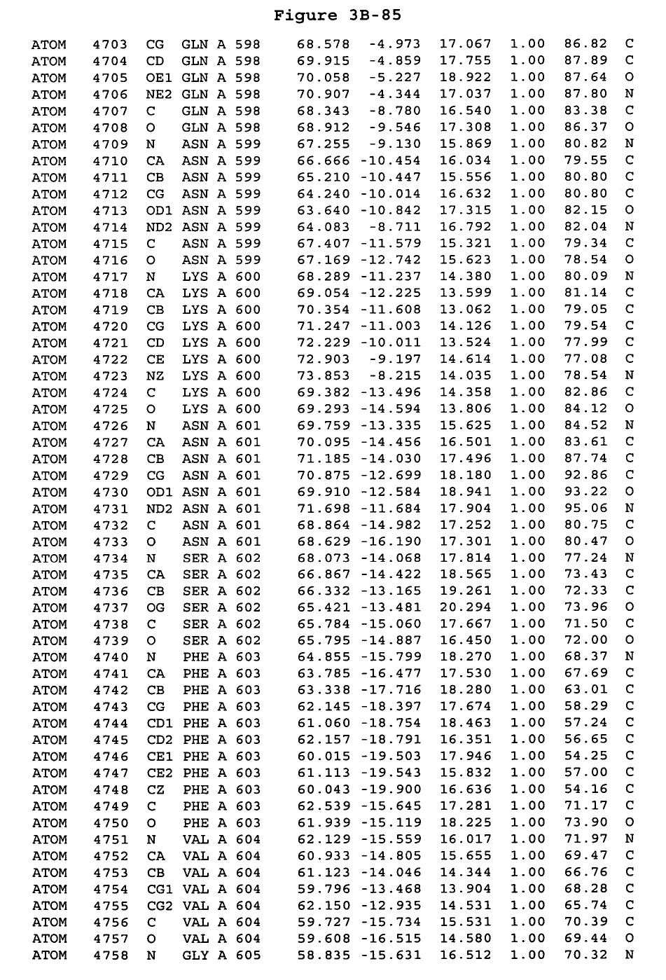

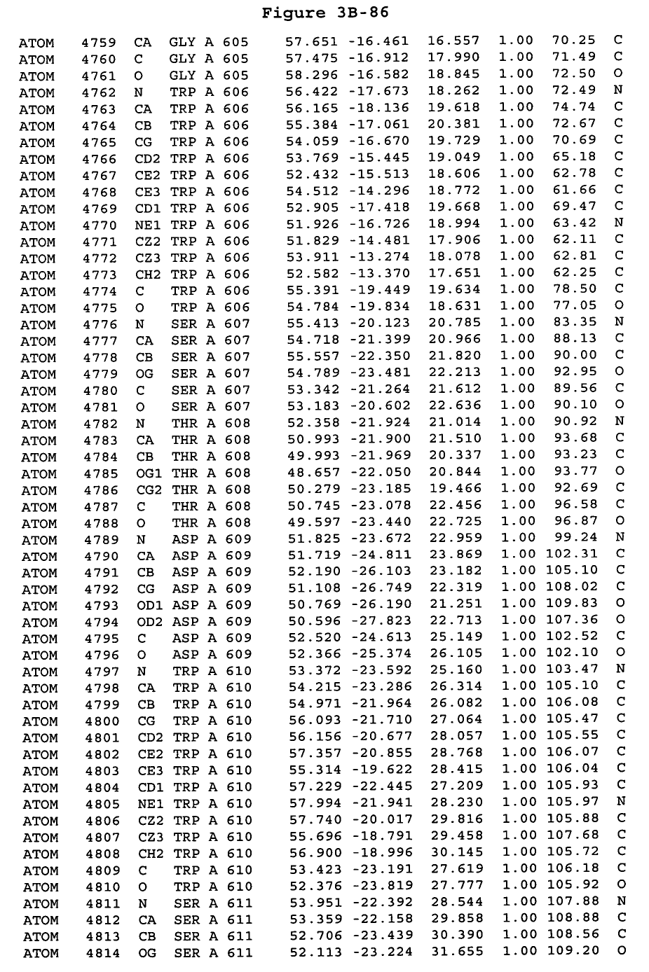

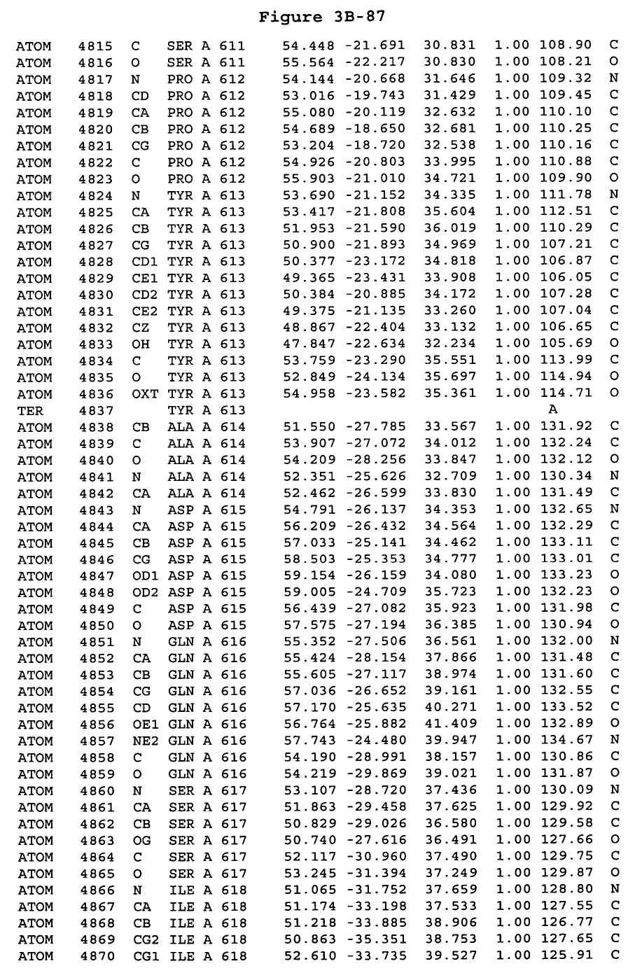

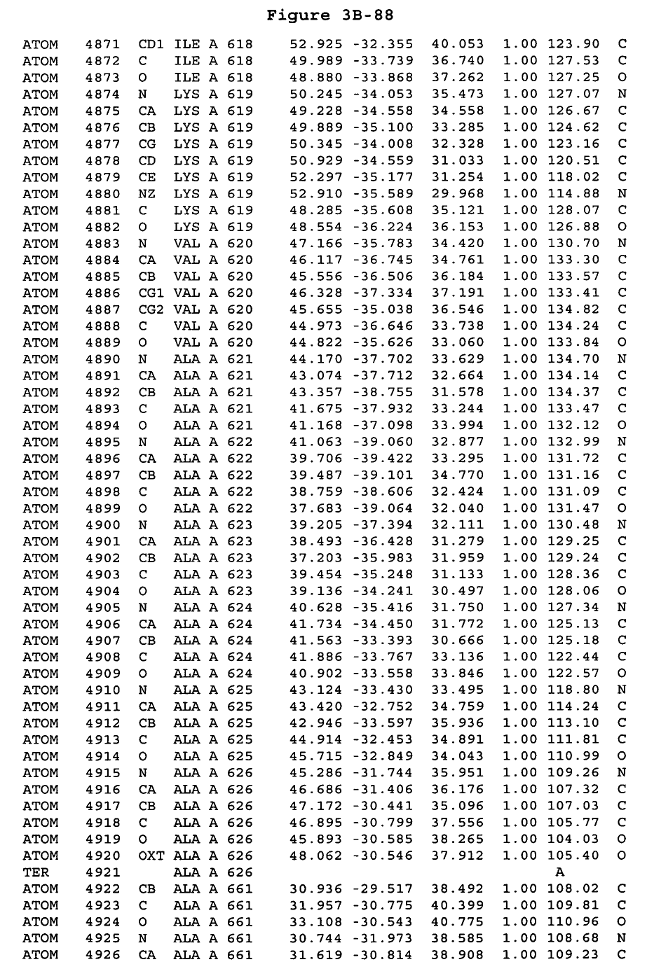

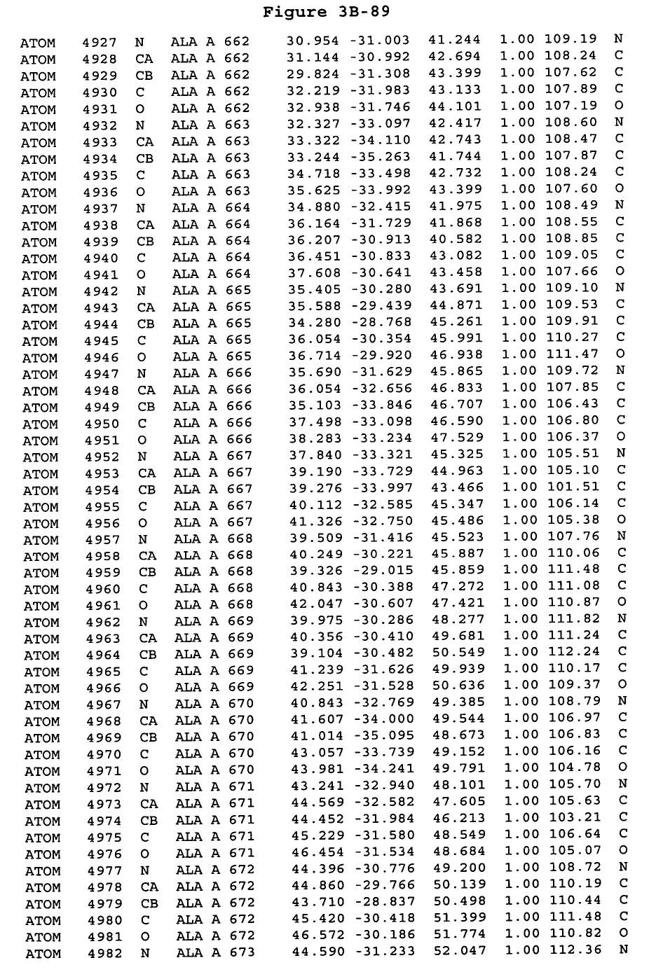

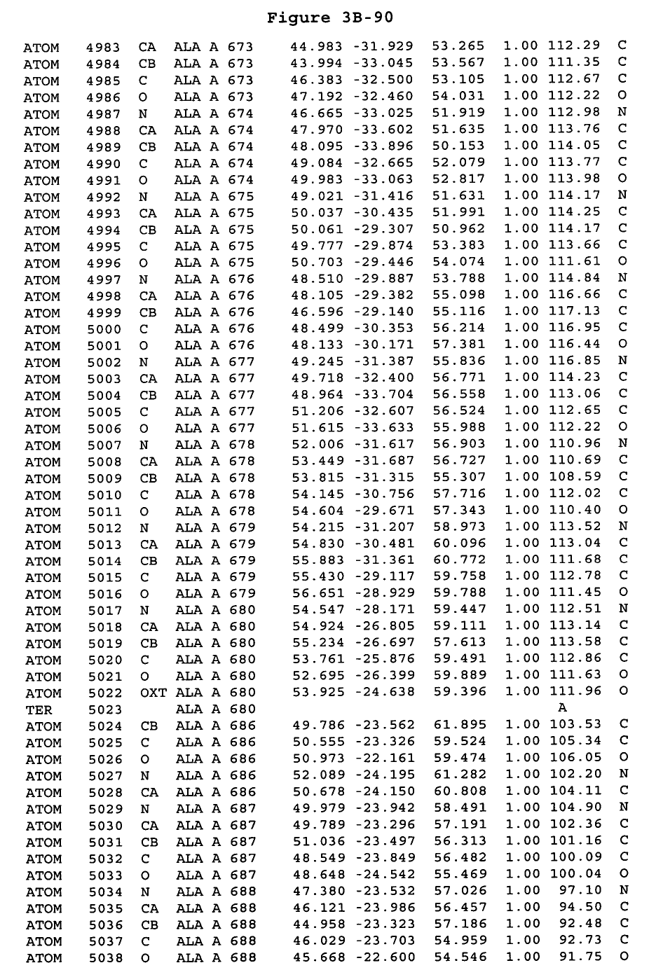

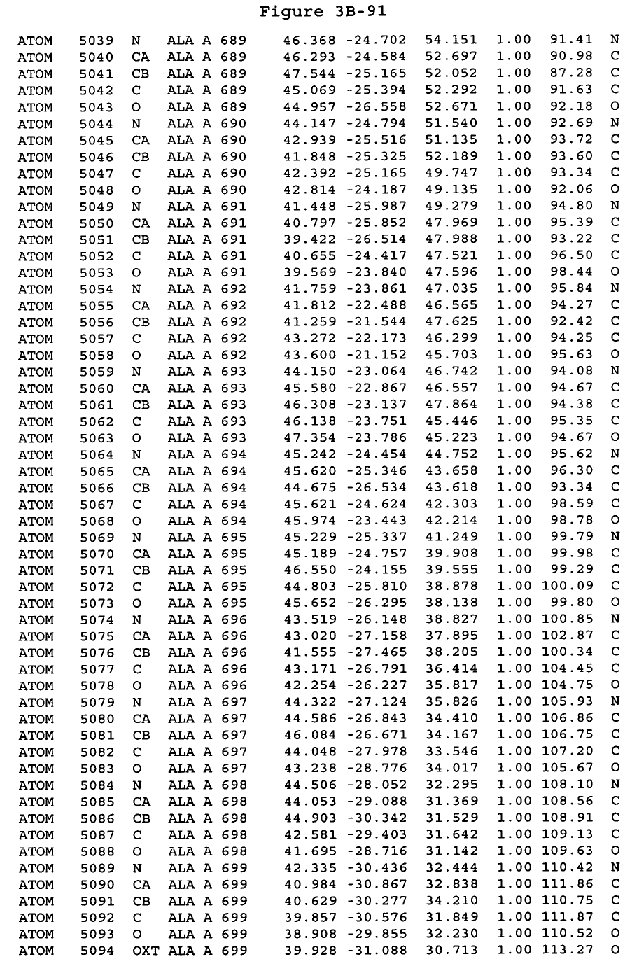

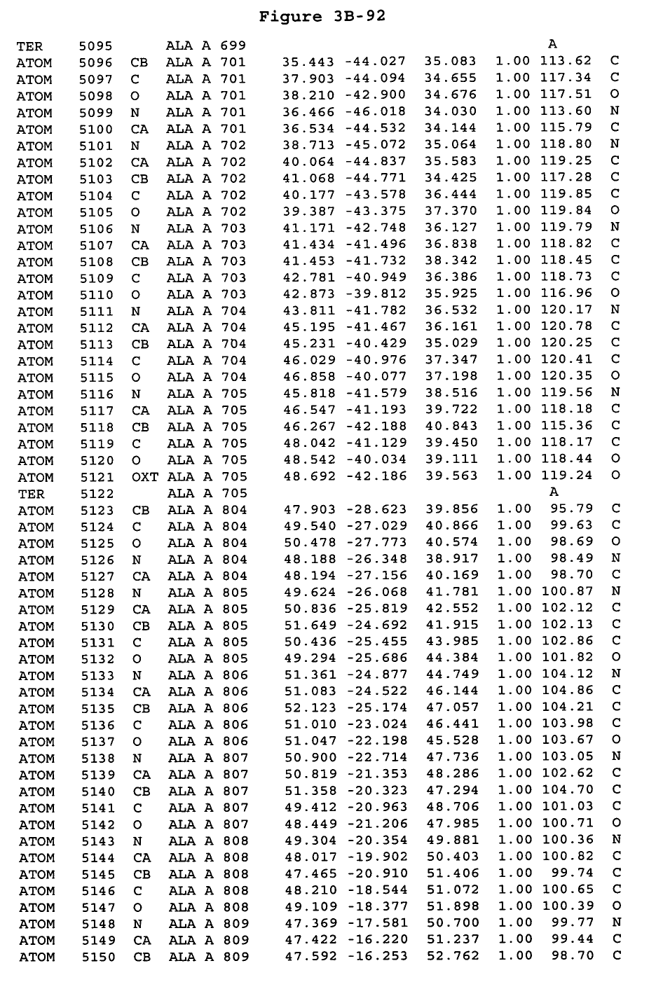

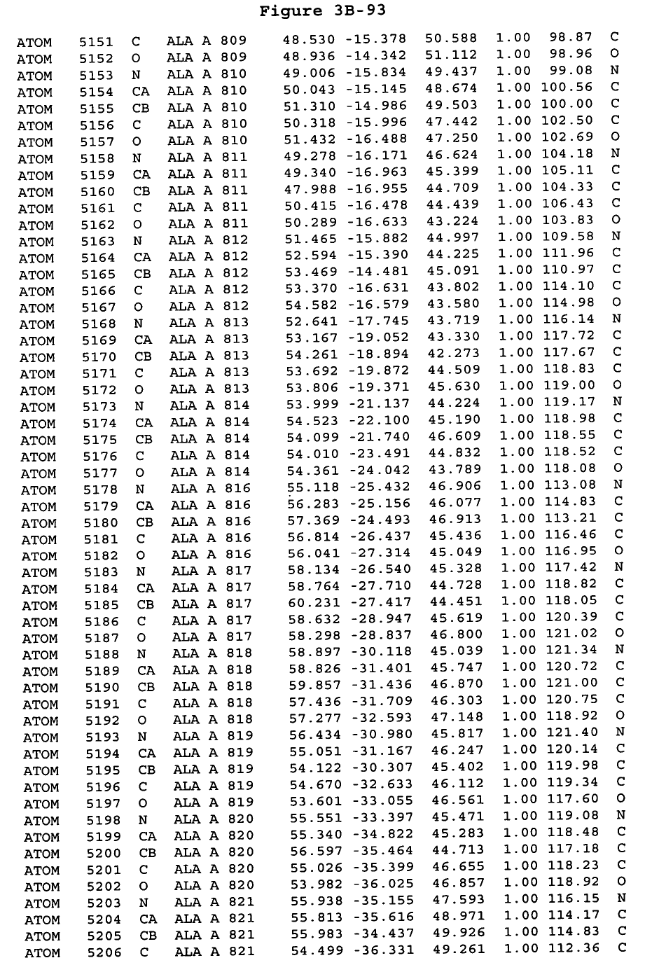

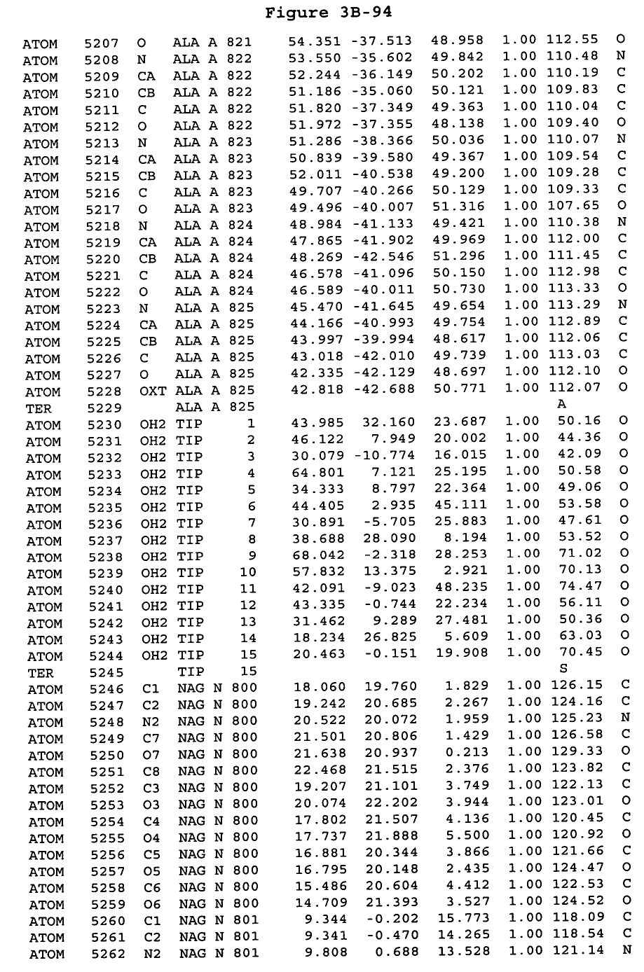

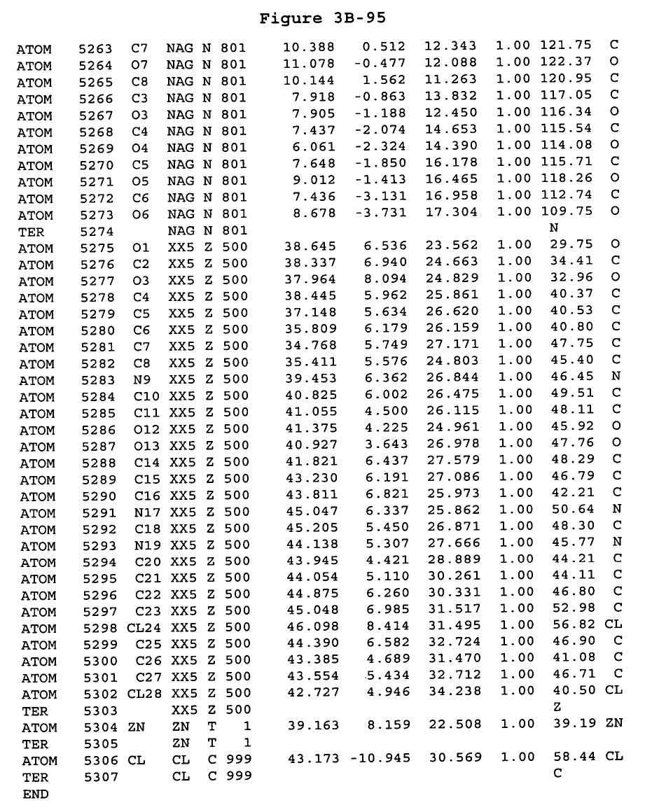

The term “ACE2-like” refers to all or a portion of a molecule or molecular complex that has a commonality of shape to all or a portion of the ACE2 protein. For example, in the ACE2-like active site binding pocket, the commonality of shape is defined by a root mean square deviation of the structure coordinates of the backbone atoms between the amino acids in the ACE2-like active site binding pocket and the ACE2 amino acids in the ACE2 active site binding pocket (as set forth in FIG. 3A or 3B). Depending on the set of ACE2 amino acids that define the ACE2 active site binding pocket, one skilled in the art would be able to locate the corresponding amino acids that define an ACE2-like active site binding pocket in a protein based on sequence or structural homology.

The term “active site” refers to the area in the ACE2 protein where the substrate binds and is cleaved by ACE2. The active site is located between the two subdomains that comprise the catalytic entity, subdomain I and II. Substrates of ACE2 include but are not limited to Angiotensin I, Angiotensin II, apelin 13, des-Arg9 bradykinin and dynorphin A 1-13. Substrates of ACE2 homologues such as ACE include but are not limited to Angiotensin I and bradykinin.

The term “associating with” refers to a condition of proximity between a chemical entity or compound, or portions thereof, and a binding pocket or binding site on a protein. The association may be non-covalent—wherein the juxtaposition is energetically favored by hydrogen bonding or van der Waals or electrostatic interactions—or it may be covalent.

The term “binding pocket” refers to a region of a molecule or molecular complex, that, as a result of its shape, favorably associates with another chemical entity or compound. The term “pocket” includes, but is not limited to, peptide or substrate binding, ATP-binding and antibody binding sites.

The term “ACE2 catalytic domain” refers to the metallopeptidase domain of human ACE2 protein This domain corresponds to the residues around 19 to 611 of SEQ ID NO:4.

The term “chemical entity” refers to chemical compounds, complexes of at least two chemical compounds, and fragments of such compounds or complexes. The chemical entity can be, for example, a ligand, a substrate, an agonist, antagonist, inhibitor, antibody, peptide, protein or drug. In one embodiment, the chemical entity is an inhibitor or substrate for the active site. In one embodiment, the inhibitor is selected from the group consisting of (S,S)2-{1-Carboxy-2-[3-(3,5-dichloro-benzyl)-3H-imidazol-4-yl]-ethylamino}-4-methyl-pentanoic acid (inhibitory), (S,S)2-{1-Carboxy-2-[3-(4-iodo-benzyl)-3H-imidazol-4-yl}-ethylamino}-4-methyl-pentanoic acid (inhibitor2), (S,S)2-[2-(6-Bromo-benzothiazol-2-ylcarbamoyl)-1-carboxy-ethylamino]-4-methyl-pentanoic acid (inhibitor3) and (S, S) 2-{1-Carboxy-2-[3-(3,5-dichloro-benzyl)-3H-imidazol-4-yl]-ethylamino}-4-phenyl-butyric acid (inhibitor4).

The term “conservative substitutions” refers to residues that are physically or functionally similar to the corresponding reference residues. That is, a conservative substitution and its reference residue have similar size, shape, electric charge, chemical properties including the ability to form covalent or hydrogen bonds, or the like. Preferred conservative substitutions are those fulfilling the criteria defined for an accepted point mutation in Dayhoff et al., Atlas of Protein Sequence and Structure, 5, pp. 345-352 (1978 & Supp.), which is incorporated herein by reference. Examples of conservative substitutions are substitutions including but not limited to the following groups: (a) valine, glycine; (b) glycine, alanine; (c) valine, isoleucine, leucine; (d) aspartic acid, glutamic acid; (e) asparagine, glutamine; (f) serine, threonine; (g) lysine, arginine, methionine; and (h) phenylalanine, tyrosine.

The term “correspond to” or “corresponding amino acids” when used in the context of amino acid residues that correspond to ACE2 amino acids refers to particular amino acids or analogues thereof in an ACE2 homologue that correspond to amino acids in the human ACE2 protein. The corresponding amino acid may be an identical, mutated, chemically modified, conserved, conservatively substituted, functionally equivalent or homologous amino acid when compared to the ACE2 amino acid to which it corresponds. For example, the following are examples of ACE2 amino acid residues that correspond to somatic ACE amino acid residues (the identity of the ACE2 residue is listed first; its position is indicated using ACE2 sequence numbering; and the identity of the sACE residue is given at the end): Y510V, P346A, T347S, P346A, T371V, E406D, R518S, F274T, R273Q, S409A, E406D, R273Q, F274T, D382F and N394E.

Methods for identifying a corresponding amino acid are known in the art and are based upon sequence, structural alignment, its functional position or a combination thereof as compared to the ACE2 protein. For example, corresponding amino acids may be identified by superimposing the backbone atoms of the amino acids in ACE2 and the protein using well known software applications, such as QUANTA (Molecular Simulations, Inc., San Diego, Calif. ©1998, 2000; Accelrys ©2001, 2002). The corresponding amino acids may also be identified using sequence alignment programs such as the “bestfit” program or CLUSTAL W Alignment Tool, supra.

The term “crystallization solution” refers to a solution which promotes crystallization comprising at least one agent including a buffer, one or more salts, a precipitating agent, one or more detergents, sugars or organic compounds, lanthanide ions, a poly-ionic compound, and/or stabilizer.

The term “complex” or “molecular complex” refers to a protein associated with a chemical entity.

The term “domain” refers to a structural unit of the ACE2 protein or homologue. The domain can comprise a binding pocket, a sequence or structural motif. In ACE2, the protein is separated into two domains: a catalytic domain comprised of two N-terminal subdomains (subdomain I and II), and a C-terminal Collectrin homology domain.

The term “fitting operation” refers to an operation that utilizes the structure coordinates of a chemical entity, binding pocket, molecule or molecular complex, or portion thereof, to associate the chemical entity with the binding pocket, molecule or molecular complex, or portion thereof. This may be achieved by positioning, rotating or translating the chemical entity in the binding pocket to match the shape and electrostatic complementarity of the binding pocket. Covalent interactions, non-covalent interactions such as hydrogen bond, electrostatic, hydrophobic, van der Waals interactions, and non-complementary electrostatic interactions such as repulsive charge-charge, dipole-dipole and charge-dipole interactions may be optimized. Alternatively, one may minimize the deformation energy of binding of the chemical entity to the binding pocket.

The term “generating a three-dimensional structure” or “generating a three-dimensional representation” refers to converting the lists of structure coordinates into structural models or graphical representation in three-dimensional space. This can be achieved through commercially or publicly available software. A model of a three-dimensional structure of a molecule or molecular complex can thus be constructed on a computer screen by a computer that is given the structure coordinates and that comprises the correct software. The three-dimensional structure may be displayed or used to perform computer modeling or fitting operations. In addition, the structure coordinates themselves, without the displayed model, may be used to perform computer-based modeling and fitting operations.

The term “homologue of ACE2” or “ACE2 homologue” refers to a molecule that has a domain having at least 40%, 60%, 80%, 90%, 95%, 96%, 97%, 98%, 99% or greater than 99% sequence identity to the catalytic domain of human ACE2 protein. Preferably, the molecule has a domain having 60%, 80%, 90%, 95%, 96%, 97%, 98%, 99% or greater than 99% sequence identity to the catalytic domain of human ACE2 protein. The homologue can be ACE2, ACE, germinal ACE, somatic ACE from human, with conservative substitutions, conservative additions or deletions thereof. The homologue can be ACE2, ACE, germinal ACE, somatic ACE from another animal species. Such animal species include, but are not limited to, mouse, rat, a primate such as monkey or other primates. The human ACE2 protein can be human ACE2 full-length protein (amino acids 1-805 of SEQ ID NO: 4); the extracellular domain with amino acids 1-740 of SEQ ID NO: 4; amino acids 1-611 of SEQ ID NO: 4; amino acid residues 19-611 of SEQ ID NO: 4. The human somatic ACE can be the full-length protein with 1306 residues, the C-terminal catalytic domain or N-terminal catalytic domain. The human germinal ACE can be the full-length protein with 732 residues or the catalytic domain. See A. J. Turner and N. M. Hooper, Trends in Pharmacological Sciences, 23, 177-183 (2002), incorporated herein by reference.

The term “homology model” refers to a structural model derived from known three-dimensional structure(s). Generation of the homology model, termed “homology modeling”, can include sequence alignment, residue replacement, residue conformation adjustment through energy minimization, or a combination thereof.

The term “motif” refers to a group of amino acids in the protein that defines a structural compartment or carries out a function in the protein, for example, catalysis or structural stabilization. The motif may be conserved in sequence, structure and function. The motif can be contiguous in primary sequence or three-dimensional space.

The term “part of a binding pocket” refers to less than all of the amino acid residues that define the binding pocket. The structure coordinates of residues that constitute part of a binding pocket may be specific for defining the chemical environment of the binding pocket, or useful in designing fragments of an inhibitor that may interact with those residues. For example, the portion of residues may be key residues that play a role in ligand binding, or may be residues that are spatially related and define a three-dimensional compartment of the binding pocket. The residues may be contiguous or non-contiguous in primary sequence.

The term “part of an ACE2 protein” refers to less than all of the amino acid residues of an ACE2 protein. In one embodiment, part of an ACE2 protein defines the binding pockets, domains or motifs of the protein. The structure coordinates of residues that constitute part of an ACE2 protein may be specific for defining the chemical environment of the protein, or useful in designing fragments of an inhibitor that may interact with those residues. The portion of residues may also be residues that are spatially related and define a three-dimensional compartment of a binding pocket, motif or domain. The residues may be contiguous or non-contiguous in primary sequence. For example, the portion of residues may be key residues that play a role in ligand or substrate binding, catalysis or structural stabilization.

The term “root mean square deviation” or “RMSD” refers to the square root of the arithmetic mean of the squares of the deviations from the mean. It is a way to express the deviation or variation from a trend or object. For purposes of this invention, the “root mean square deviation” defines the variation in the backbone of a protein from the backbone of ACE2 or a binding pocket portion thereof, as defined by the structure coordinates of ACE2 described herein. It would be readily apparent to those skilled in the art that the calculation of RMSD involves standard error.

The term “soaked” refers to a process in which the crystal is transferred to a solution containing the compound of interest.

The term “structure coordinates” refers to Cartesian coordinates derived from mathematical equations related to the patterns obtained on diffraction of a monochromatic beam of X-rays by the atoms (scattering centers) of a protein or protein complex in crystal form. The diffraction data are used to calculate an electron density map of the repeating unit of the crystal. The electron density maps are then used to establish the positions of the individual atoms of the enzyme or enzyme complex.

The term “subdomain” refers to a portion of the above-defined domain. The metallopeptidase domain of ACE2 is a bi-lobal structure consisting of N-terminal and C-terminal subdomains. The N-terminal and zinc containing subdomain is comprised of residues 19-102, 290-397, and 417-430 and is called subdomain I. The C-terminal subdomain is comprised of residues 103-289, 398-416, and 431-613 and is called subdomain II.

The term “substantially all of an ACE2 binding pocket” or “substantially all of an ACE2 protein” refers to all or almost all of the amino acids in the ACE2 binding pocket or protein. For example, substantially all of an ACE2 binding pocket can be 100%, 95%, 90%, 80%, or 70% of the residues defining the ACE2 binding pocket or protein.

The term “substantially pure” refers to a protein isolated to a purity which is more than 90% pure. In one embodiment, the protein is at least 95% pure. In one embodiment, the protein is at least 99% pure.

The term “sufficiently homologous to ACE2” refers to a protein that has a sequence identity of at least 25% compared to ACE2 protein. In one embodiment, the sequence identity is at least 40%. In other embodiments, the sequence identity is at least 50%, 60%, 70%, 80%, 90%, 95%, 96%, 97%, 98% or 99%.

The term “three-dimensional structural information” refers to information obtained from the structure coordinates. Structural information generated can include the three-dimensional structure or graphical representation of the structure. Structural information can also be generated when subtracting distances between atoms in the structure coordinates, calculating chemical energies for an ACE2 molecule or molecular complex or homologues thereof, calculating or minimizing energies for an association of an ACE2 molecule or molecular complex or homologues thereof to a chemical entity.

Crystallizable Compositions and Crystals of ACE2 Protein and Protein Complexes

In one embodiment, the invention provides a crystallizable composition comprising ACE2 protein or its homologue. In certain embodiments, the crystallizable composition comprising ACE2 or its homologue further comprises between about 8 to 30% v/v of precipitant polyethylene glycol, a buffer that maintains pH between about 4.0 and 8.5, and 100-300 mM MgCl₂. In other embodiments, the crystallizable composition comprises ACE2 protein, 13 or 14% PEG 8000, 100 mM Tris-HCl at pH 8.5 and 200 mM MgCl₂. In yet other embodiments, the crystallizable composition comprises ACE2 or its homologue and a precipitant that is PEG 4000 or PEG 400. In certain embodiments, the crystallizable composition comprises ACE2 or its homologue and a salt that is sodium acetate, lithium sulfate or cadmium chloride. In certain embodiments, the crystallizable composition comprises ACE2 protein, 14% PEG 8000, 100 mM Tris-HCl at pH 8.5 and 200 mM MgCl₂. In certain embodiments, the invention provides a crystallizable composition comprising human ACE2 protein, a fragment thereof or a homologue thereof.

In another embodiment, the invention provides a crystallizable composition comprising an ACE2 protein or homologue thereof and a chemical entity. In one embodiment, the crystallizable composition comprises ACE2 or its homologue and a chemical entity that is any suitable inhibitor or substrate for the active site of ACE2 or its homologue. In particular embodiments, the crystallizable composition comprises ACE2 or its homologue and an inhibitor for the active site that is selected from the group consisting of (S,S)2-{1-carboxy-2-[3-(3,5-dichloro-benzyl)-3H-imidazol-4-yl]-ethylamino}-4-methyl-pentanoic acid, (S,S)2-{1-Carboxy-2-[3-(4-iodo-benzyl)-3H-imidazol-4-yl}-ethylamino}-4-methyl-pentanoic acid, (S,S)2-[2-(6-Bromo-benzothiazol-2-ylcarbamoyl)-1-carboxy-ethylamino]-4-methyl-pentanoic acid and (S, S) 2-{1-Carboxy-2-[3-(3,5-dichloro-benzyl)-3H-imidazol-4-yl]-ethylamino}-4-phenyl-butyric acid. In one embodiment, the crystallizable composition comprising ACE2 or its homologue further comprises between about 10-30% v/v polyethylene glycol, a buffer that maintains pH between about 6.0 and 8.5, and 300-800 mM NaCl. In certain embodiments, the crystallizable composition comprises an ACE2 protein-inhibitor complex, between about 14-25% PEG, 100 mM Tris HCl pH 7.0 to 7.5 and 300-800 mM NaCl. In other embodiments, the crystallizable composition comprises an ACE2 protein-inhibitor complex, between about 19% PEG 3000, 100 mM Tris HCl pH 7.5 and 600 mM NaCl. In certain embodiments, the invention provides a crystallizable composition comprising human ACE2 protein, a fragment thereof or a homologue thereof, wherein said composition further comprises a chemical entity.

The invention provides a substantially pure ACE2 protein or homologue thereof. In certain embodiments, the ACE2 protein or its homologue is more than 90% pure. In other embodiments, the ACE2 protein or its homologue is at least 95% pure. In yet other embodiments, the ACE2 protein or its homologues is at least 99% pure. In certain embodiments, the ACE2 protein is human ACE2 protein, a fragment thereof or a homologue thereof.

According to another embodiment, the invention provides a crystal composition comprising ACE2 protein or its homologue, the ACE2 optimally being human ACE2, a fragment thereof or a homologue thereof. In another embodiment, the invention provides a crystal composition comprising ACE2 protein or its homologue and a chemical entity, the ACE2 optimally being human ACE2, a fragment thereof or a homologue thereof. In certain embodiments, the crystallizable composition comprises ACE2 or its homologue and a chemical entity that is an inhibitor or substrate for the active site. Preferably, the native crystal has a unit cell dimension of a=103.7 Å b=89.6 Å c=112.4 Å, β=109.1° and belongs to space group C2. In another preferred embodiment, the complex crystal has a unit cell dimension of a=100.7 Å b=86.8 Å c=105.7 Å, β=103.6° and belongs to space group C2. It will be readily apparent to those skilled in the art that the unit cells of the crystal compositions may deviate ±1-2 Å from the above cell dimensions depending on the deviation in the unit cell calculations.

As used herein, the ACE2 protein in the crystallizable or crystal compositions can be full-length human ACE2 protein (amino acids 1-805 of SEQ ID NO: 4); an extracellular domain of human ACE2 protein (amino acids 1-740 of SEQ ID NO: 4; amino acids 1-611 of SEQ ID NO:4; amino acid residues 19-611 of SEQ ID NO: 4); or the aforementioned with conservative substitutions, deletions or additions, to the extent that the protein substitutions, deletions or additions maintains an ACE2 activity, preferably the protein with substitutions, deletions or additions is at least 40%, 50%, 60%, 70%, 80%, 90%, 95%, 96%, 97%, 98%, or 99% identical to one of the aforementioned. Preferably, the protein with substitutions, deletions or additions is at least 60%, 70%, 80%, 90%, 95%, 96%, 97%, 98%, or 99% identical to one of the aforementioned.

The ACE2 protein or its homologue may be produced by any well-known method, including synthetic methods, such as solid phase, liquid phase and combination solid phase/liquid phase syntheses; recombinant DNA methods, including cDNA cloning, optionally combined with site directed mutagenesis; and/or purification of the natural products.

Methods of Obtaining Crystals of ACE2 or Its Homologues

The invention also relates to a method of obtaining a crystal of an ACE2 protein or homologue thereof, comprising the steps of:

a) producing and purifying ACE2 protein or homologue thereof;

b) combining a crystallization solution with said ACE2 protein to produce a crystallizable composition; and

c) subjecting the composition to conditions which promote crystallization.

The invention also relates to a method of obtaining a crystal of an ACE2 protein complex or homologue thereof, comprising the steps of:

a) producing and purifying ACE2 protein or homologue thereof;

b) combining said ACE2 protein, or a homologue thereof, in the presence or absence of a chemical entity with a crystallizable solution to produce a crystallizable composition; and

c) subjecting the composition to conditions which promote crystallization.

In certain embodiments of the methods of obtaining crystals, the protein complex comprises ACE2 or its homologue and a chemical entity that binds to the active site of ACE2 or its homologue.

In certain embodiments, the method of making crystals of ACE2 proteins or a homologue thereof in the presence or absence of a chemical entity includes the use of a device for promoting crystallizations. Devices for promoting crystallization can include but are not limited to the hanging-drop, sitting-drop, sandwich-drop, dialysis, microbatch or microtube batch devices (U.S. Pat. Nos. 4,886,646, 5,096,676, 5,130,105, 5,221,410 and 5,400,741; Pav et al., Proteins: Structure, Function, and Genetics, 20, pp. 98-102 (1994); Chayen, Acta. Cryst., D54, pp. 8-15 (1998), Chayen, Structure, 5, pp. 1269-1274 (1997), D'Arcy et al., J. Cryst. Growth, 168, pp. 175-180 (1996) and Chayen, J. Appl. Cryst., 30, pp. 198-202 (1997), incorporated herein by reference). The hanging-drop, sitting-drop and some adaptations of the microbatch methods (D'Arcy et al., J. Cryst. Growth, 168, pp. 175-180 (1996) and Chayen, J. Appl. Cryst., 30, pp. 198-202 (1997)) produce crystals by vapor diffusion. The hanging drop and sitting drop containing the crystallizable composition is equilibrated against a reservoir containing a higher or lower concentration of precipitant. As the drop approaches equilibrium with the reservoir, the saturation of protein in the solution leads to the formation of crystals.

Microseeding may be used to increase the size and quality of crystals. In this instance, micro-crystals are crushed to yield a stock seed solution. The stock seed solution is diluted in series. Using a needle, glass rod or strand of hair, a small sample from each diluted solution is added to a set of equilibrated drops containing a protein concentration equal to or less than a concentration needed to create crystals without the presence of seeds. The aim is to end up with a single seed crystal that will act to nucleate crystal growth in the drop.

It would be readily apparent to one of skill in the art to vary the crystallization conditions disclosed above to identify other crystallization conditions that would produce crystals of ACE2 protein or a homologue thereof in the presence or absence of a chemical entity. Such variations include, but are not limited to, adjusting pH, protein concentration and/or crystallization temperature, changing the identity or concentration of salt and/or precipitant used, using a different method for crystallization, or introducing additives such as detergents (e.g., TWEEN 20 (monolaurate), LDOA, Brji 30 (4 lauryl ether)), sugars (e.g., glucose, maltose), organic compounds (e.g., dioxane, dimethylformamide), lanthanide ions, or poly-ionic compounds that aid in crystallizations. High throughput crystallization assays may also be used to assist in finding or optimizing the crystallization condition.

Binding Pockets of ACE2 Protein or Its Homologues

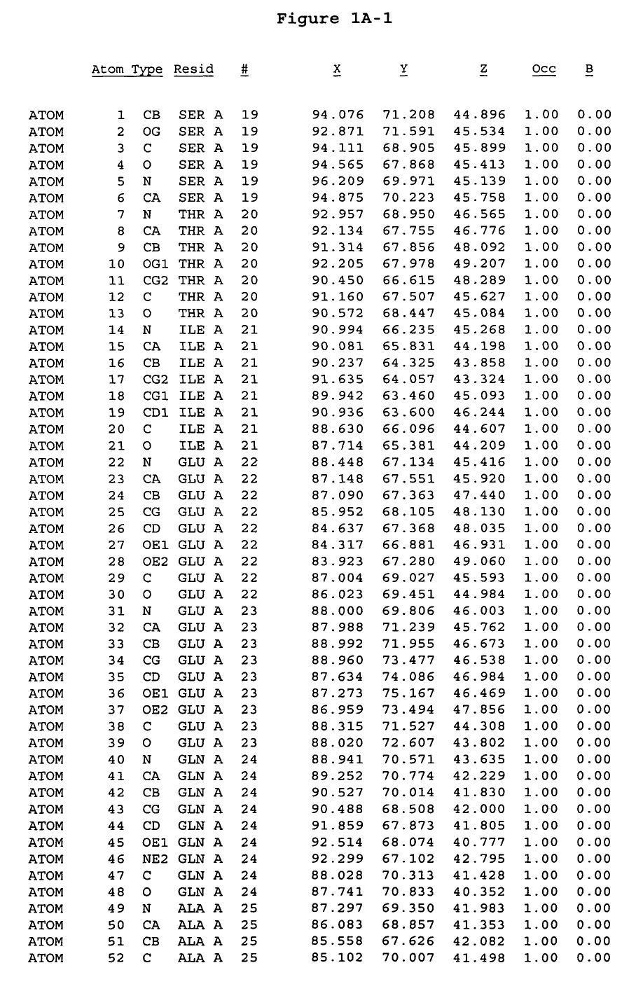

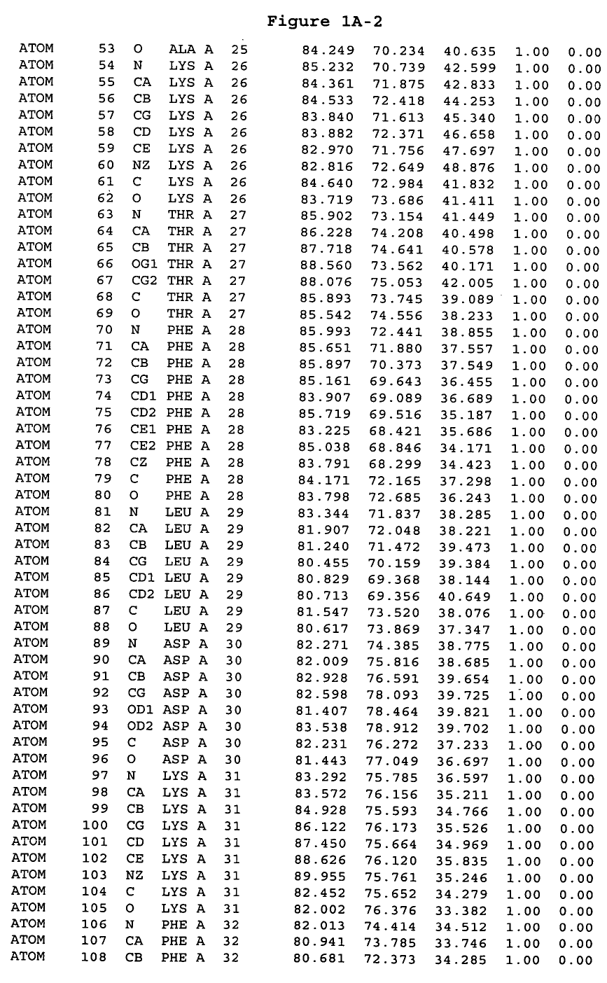

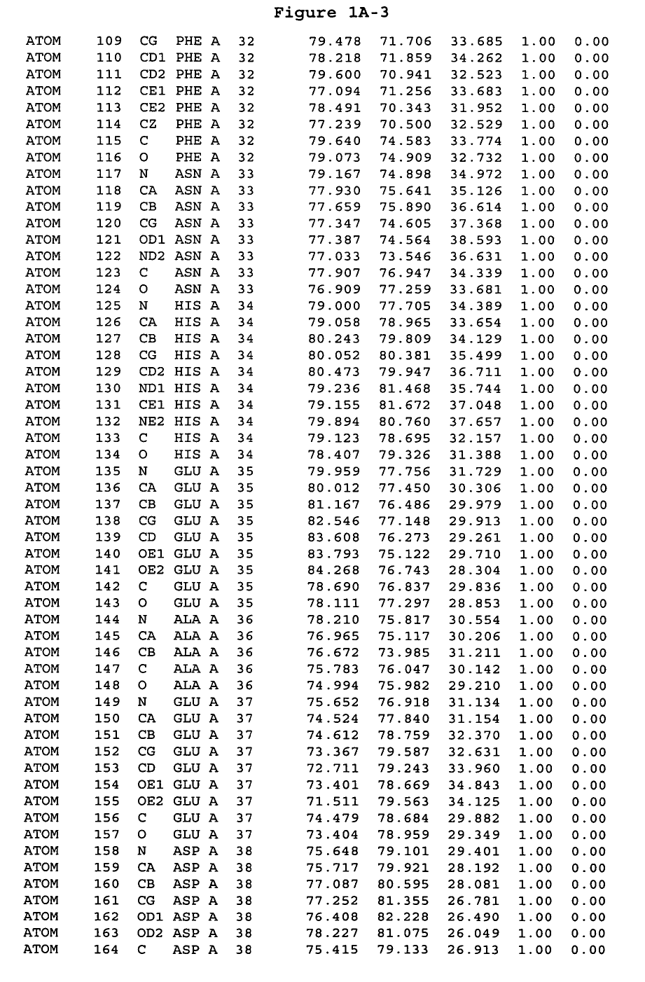

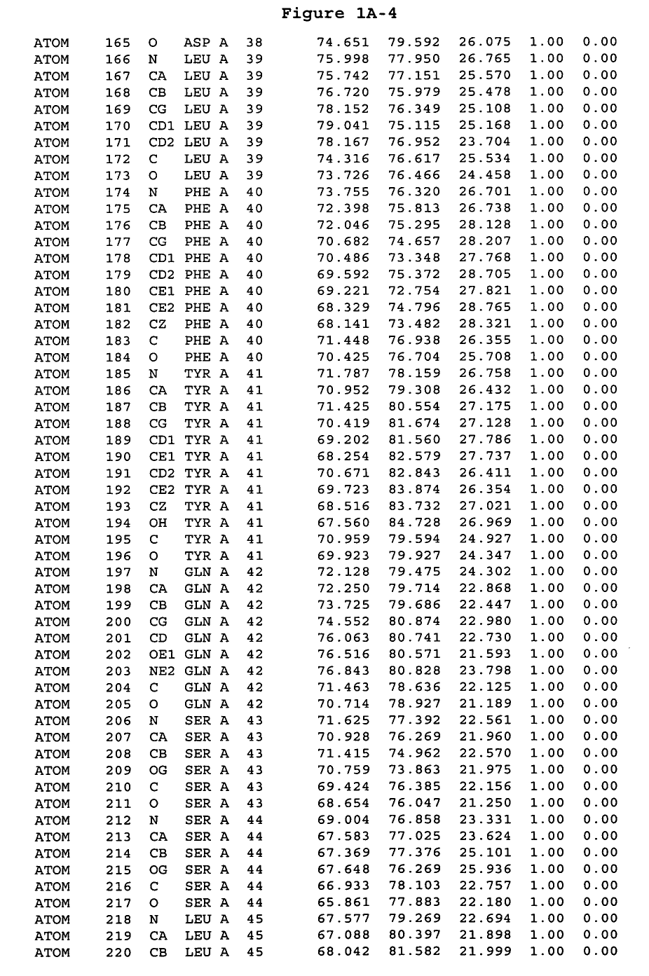

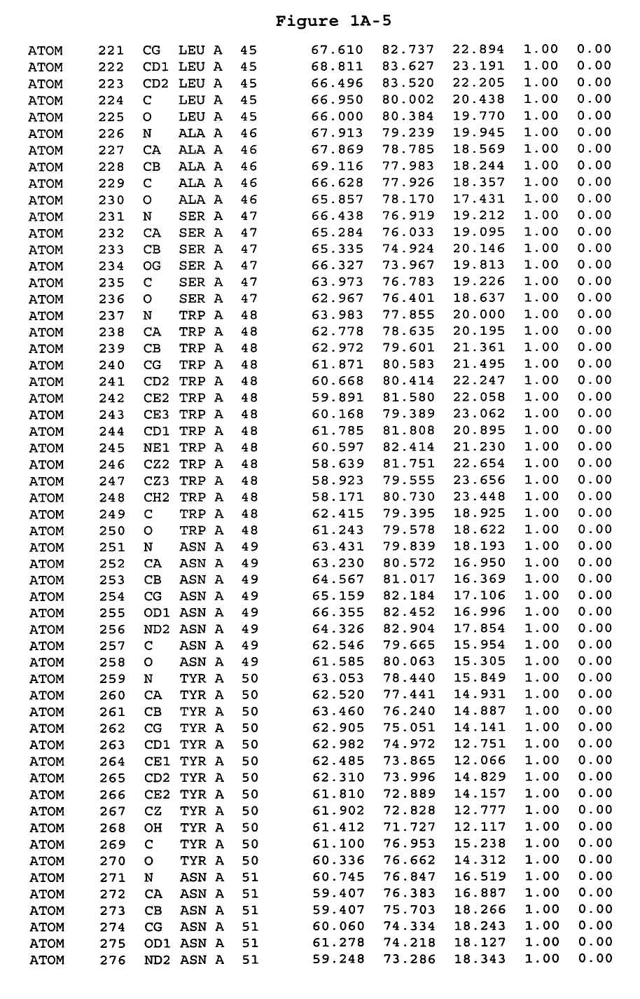

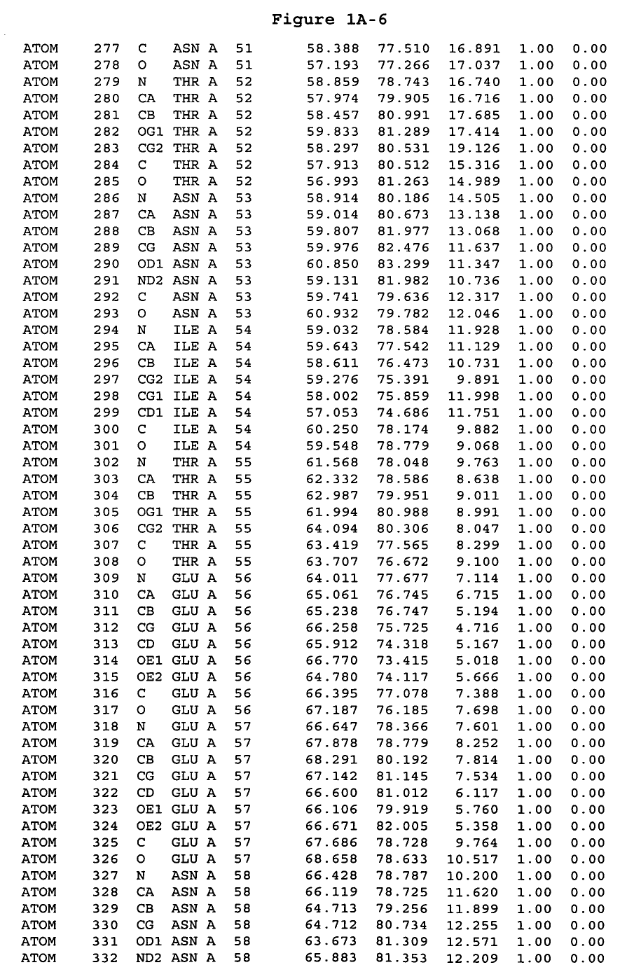

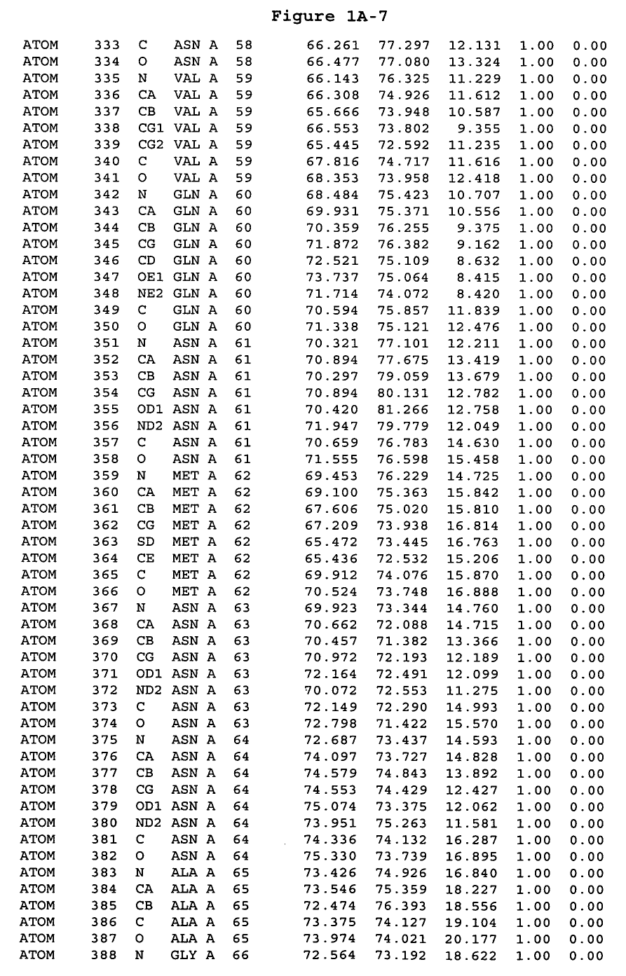

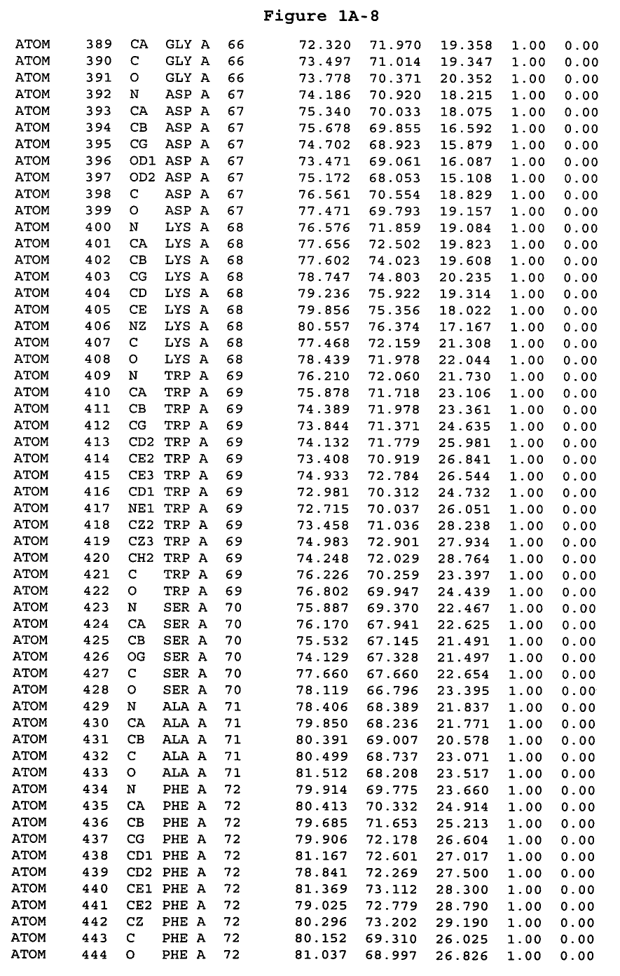

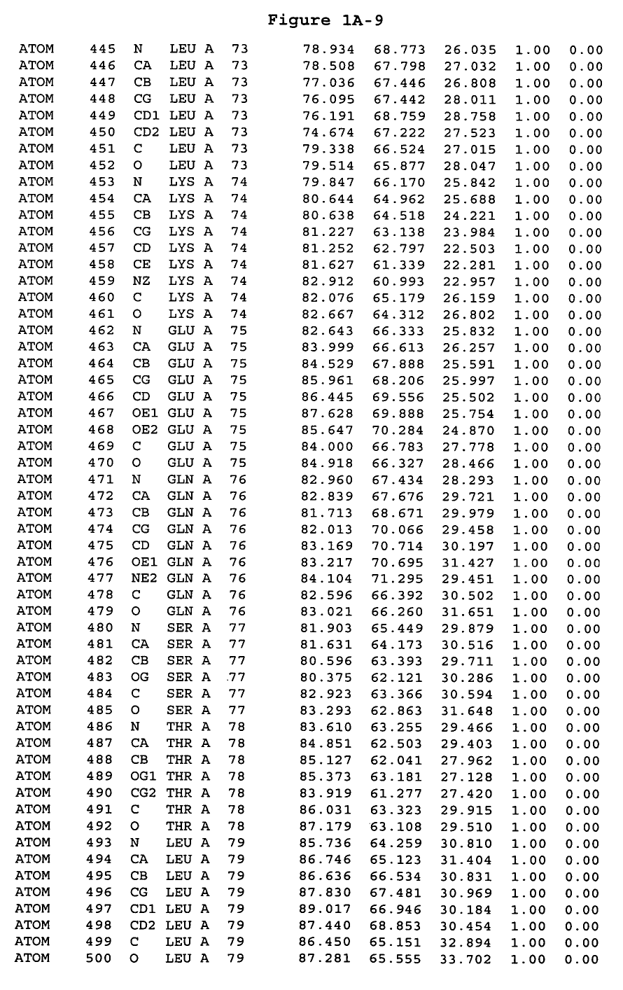

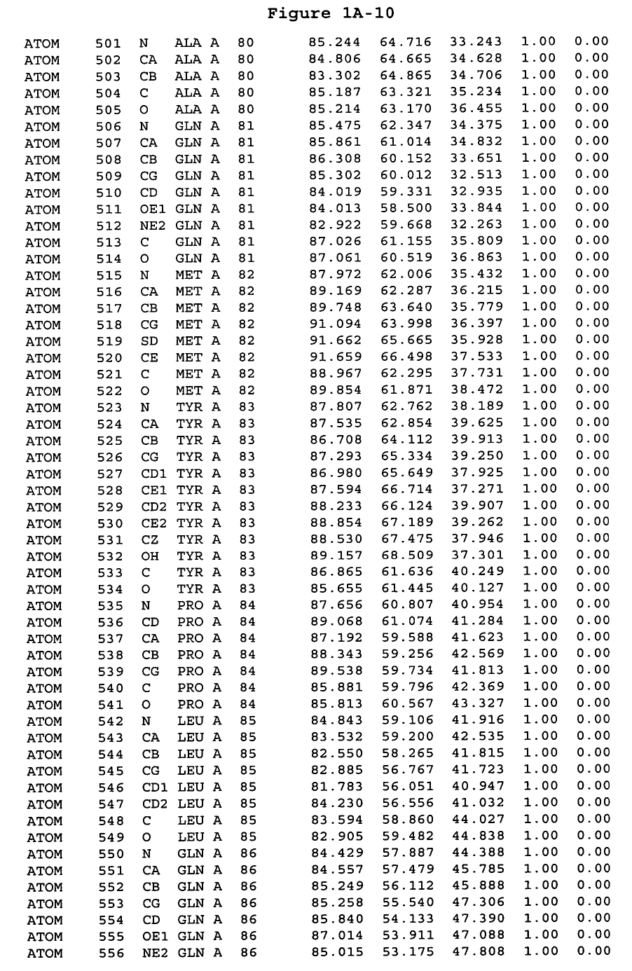

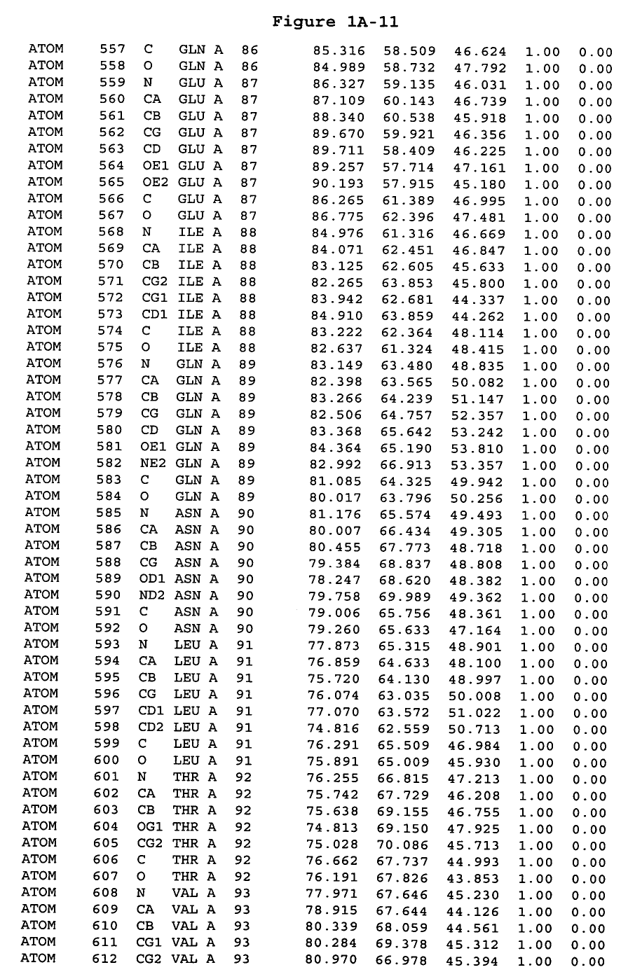

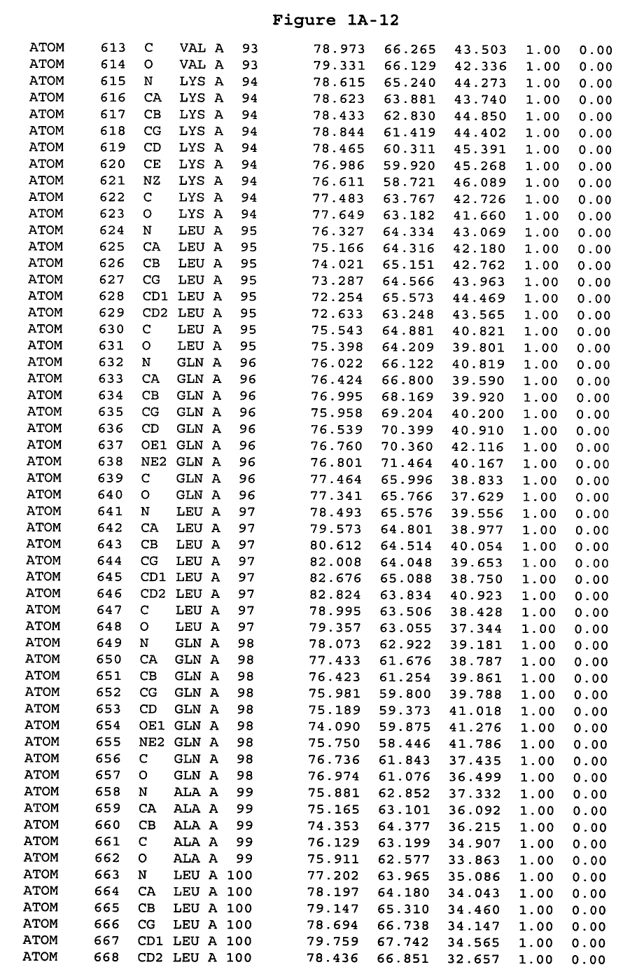

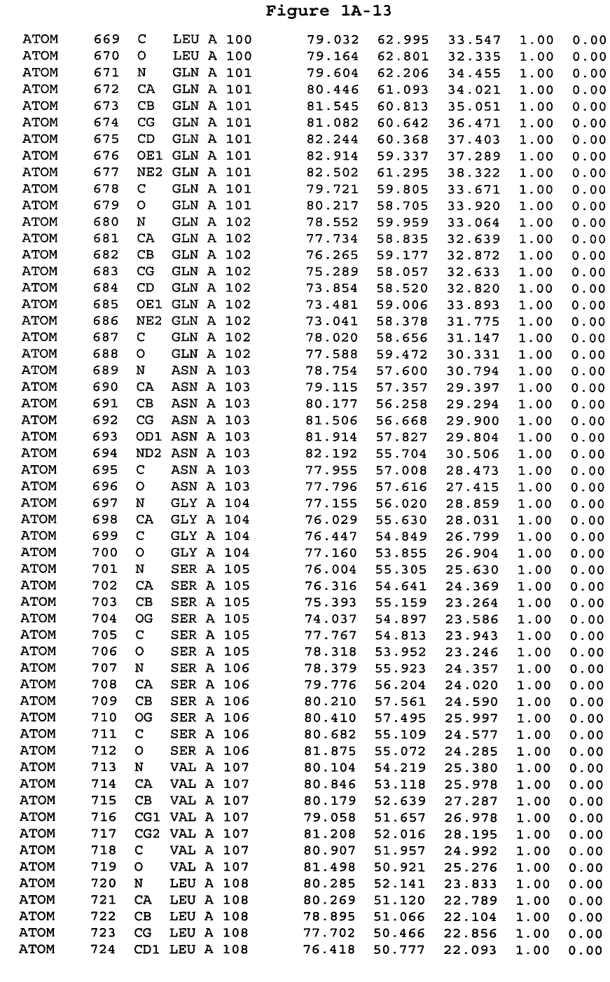

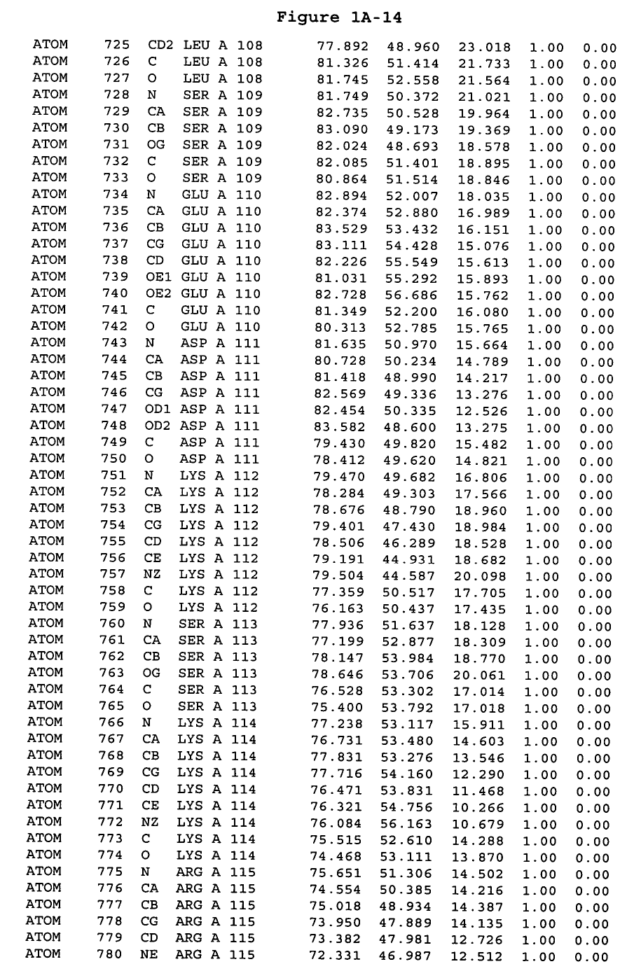

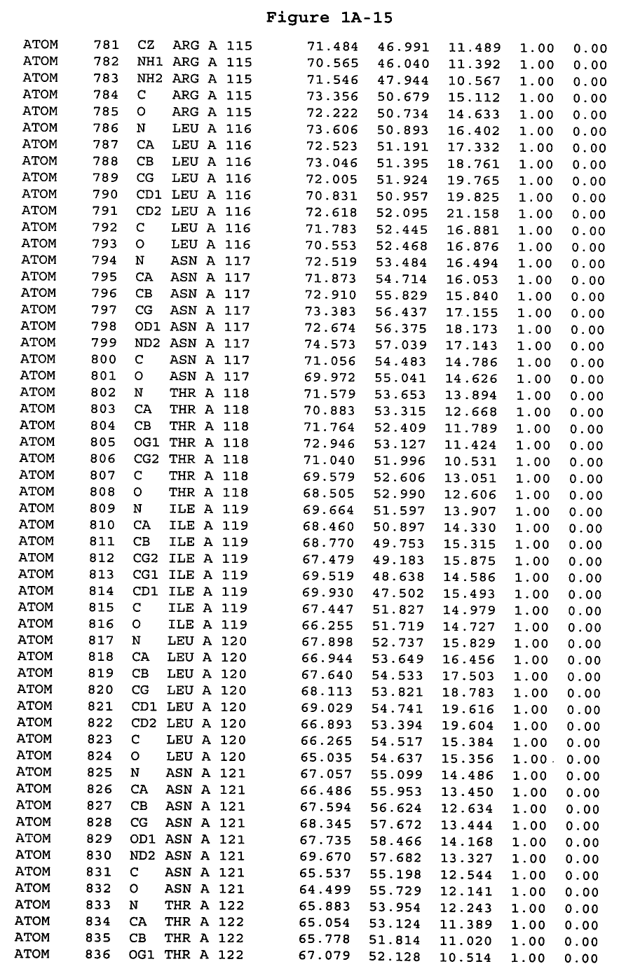

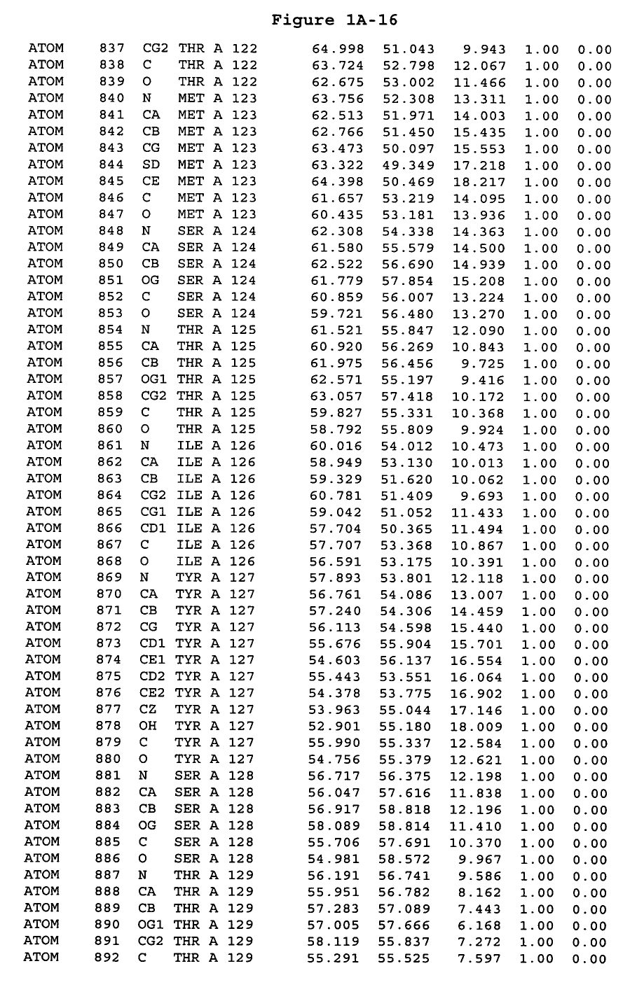

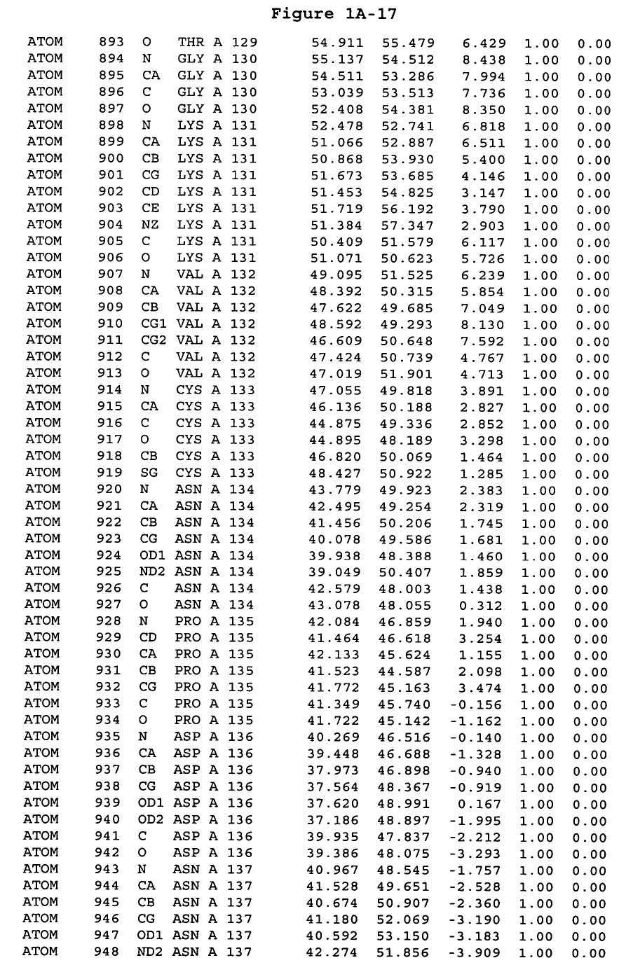

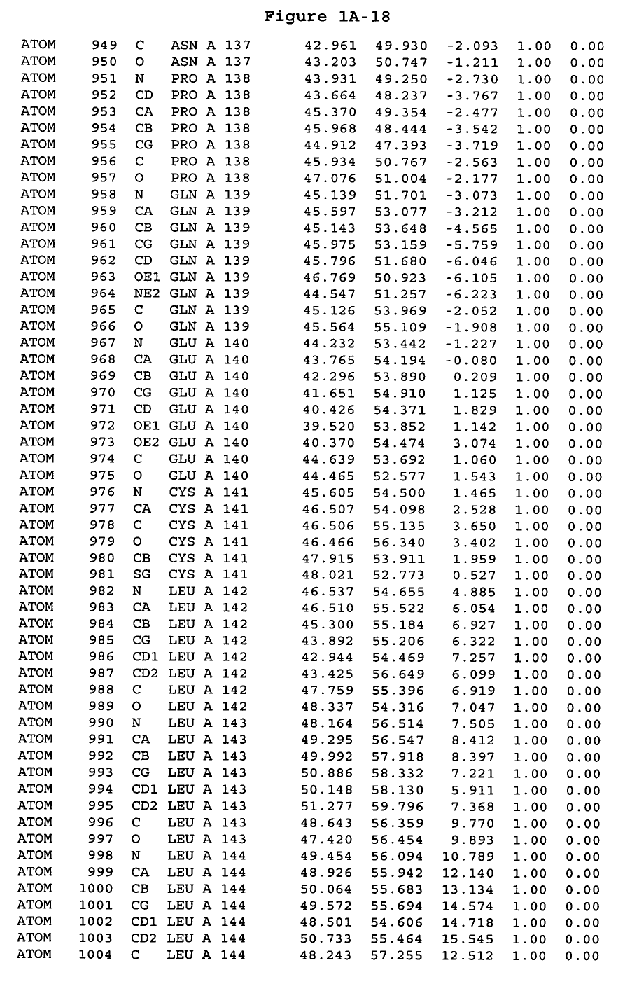

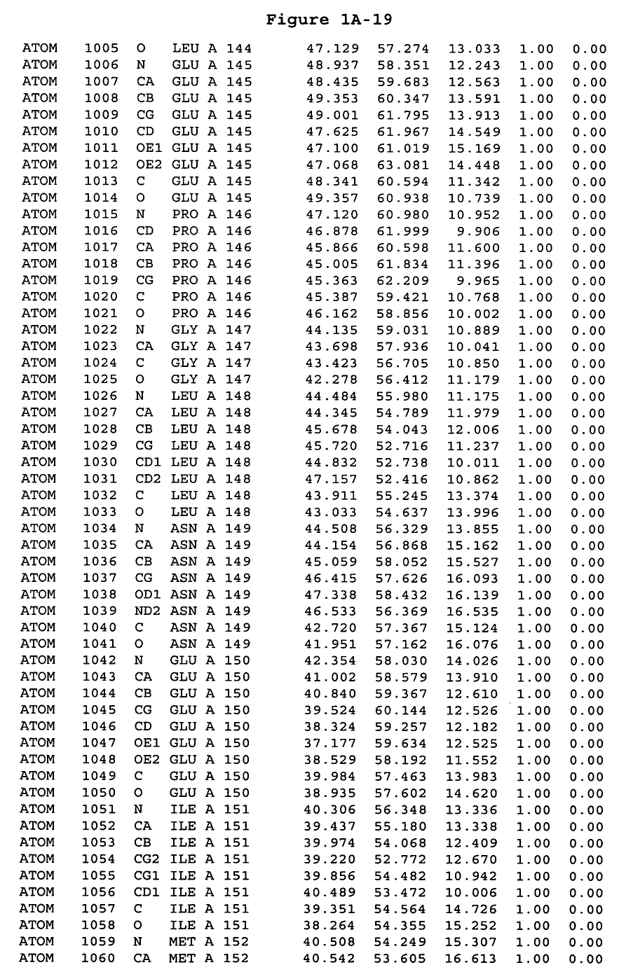

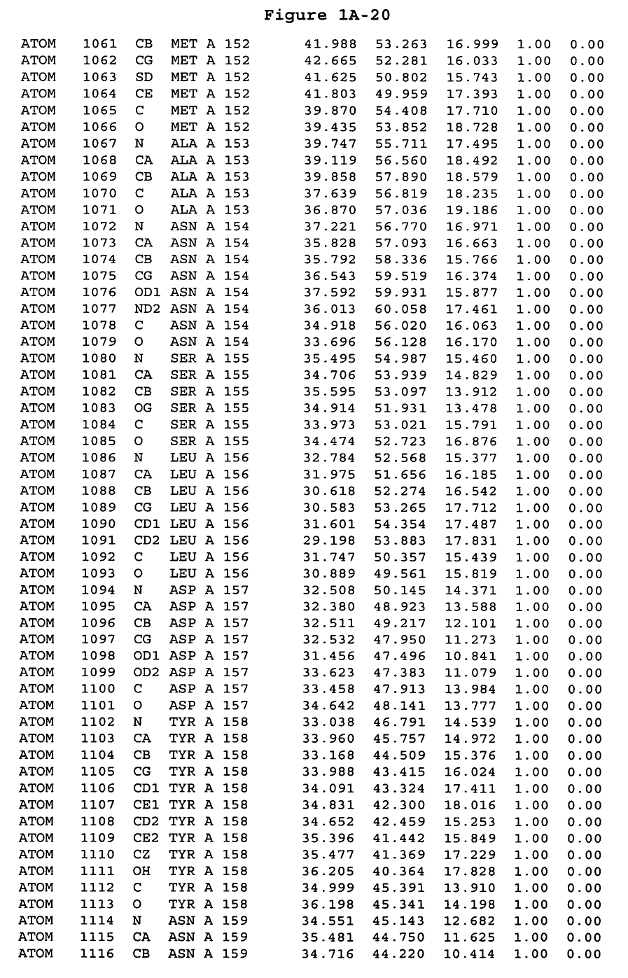

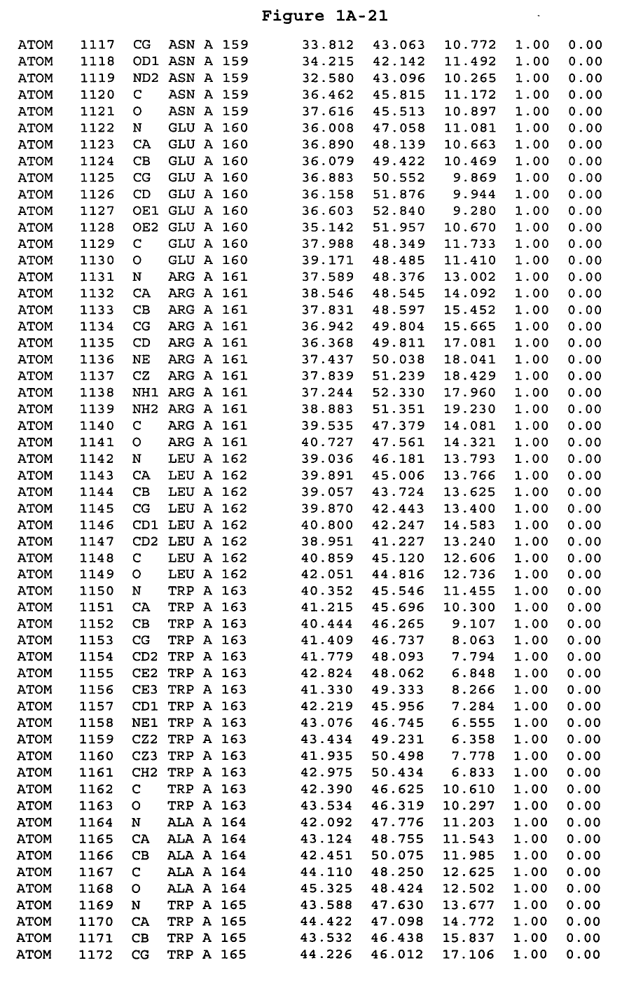

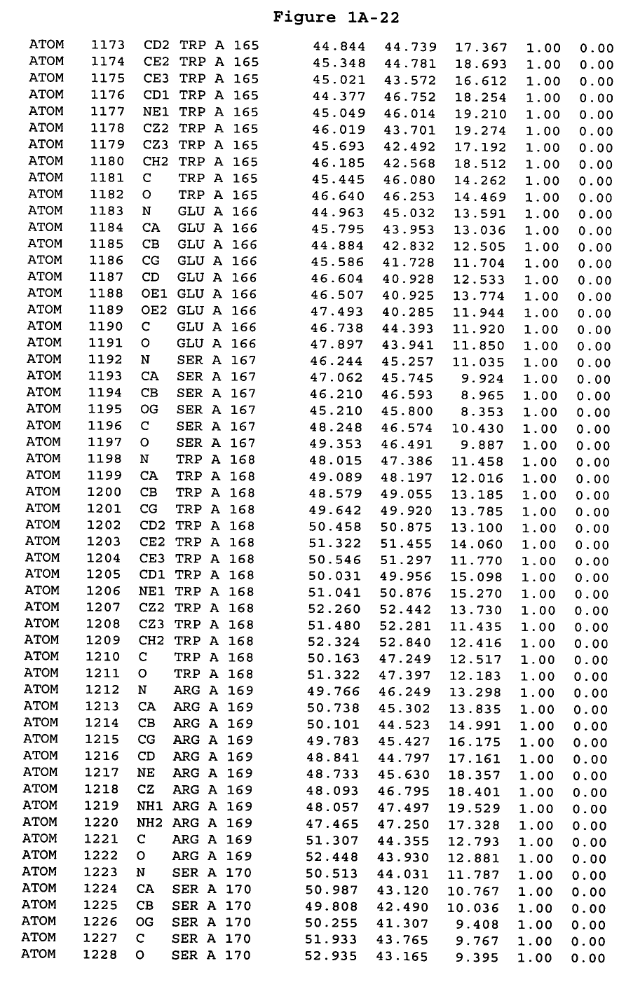

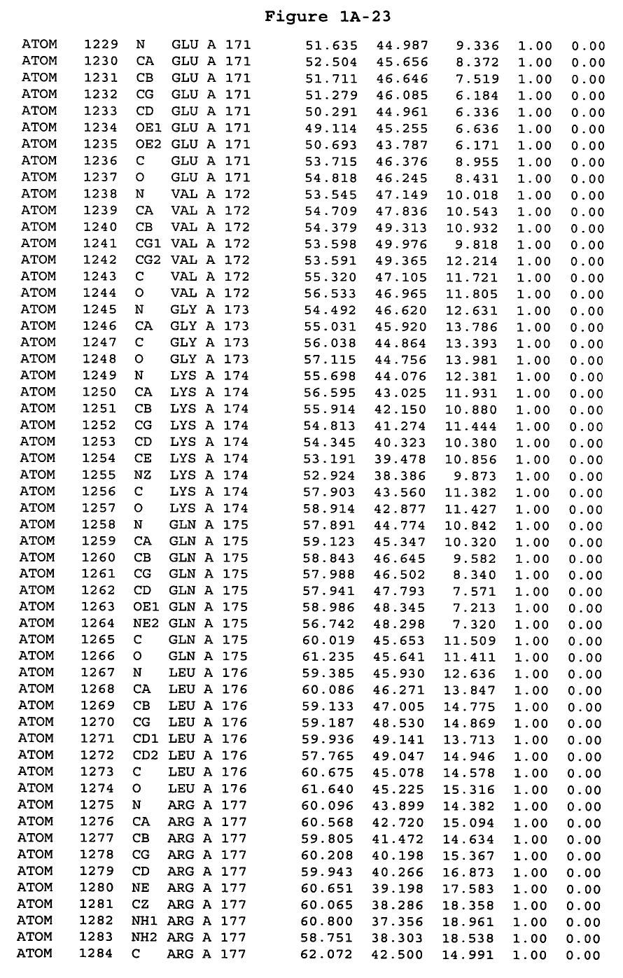

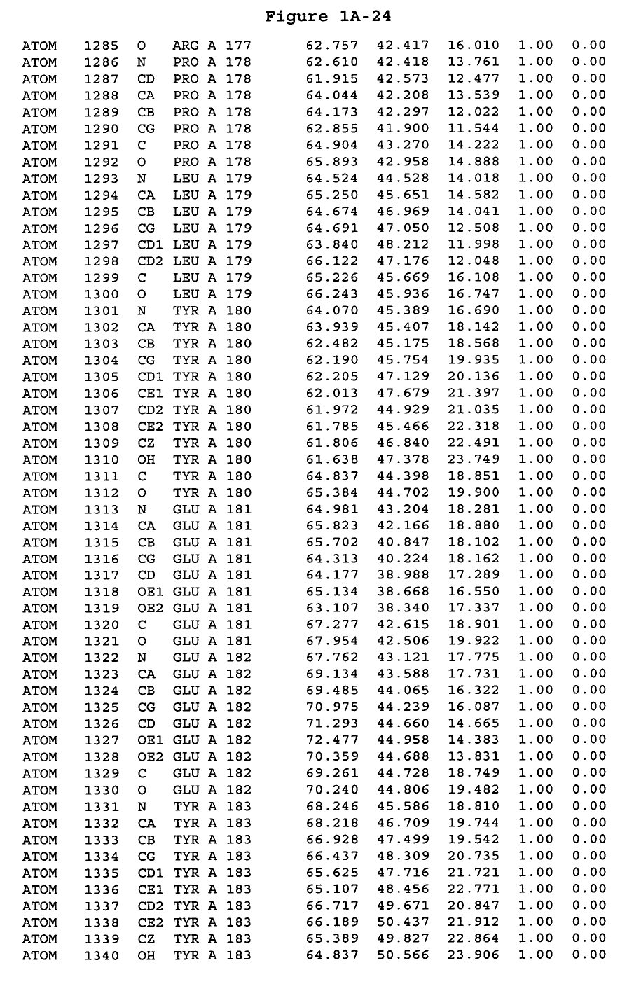

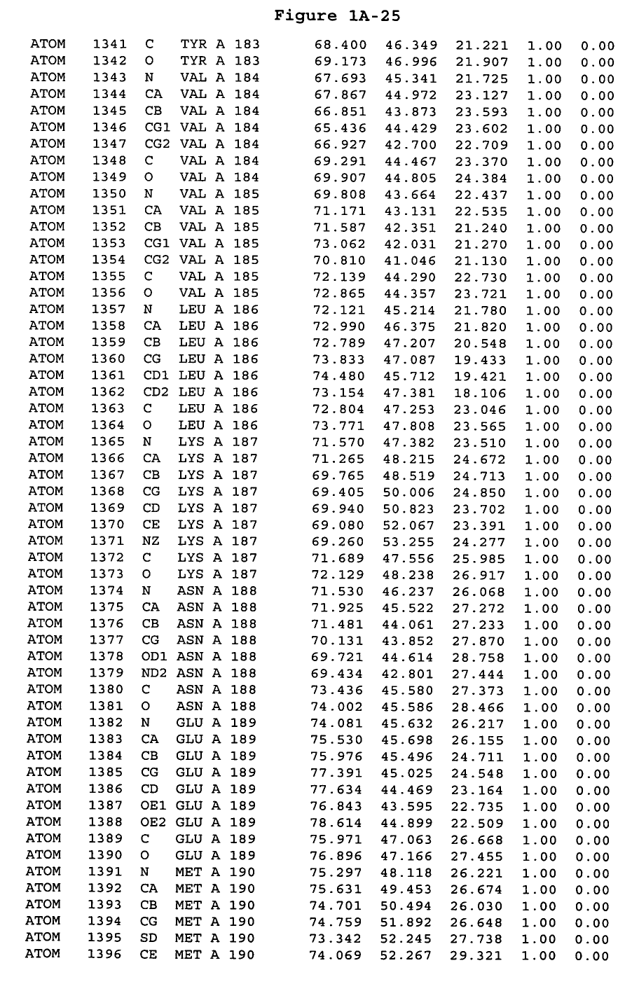

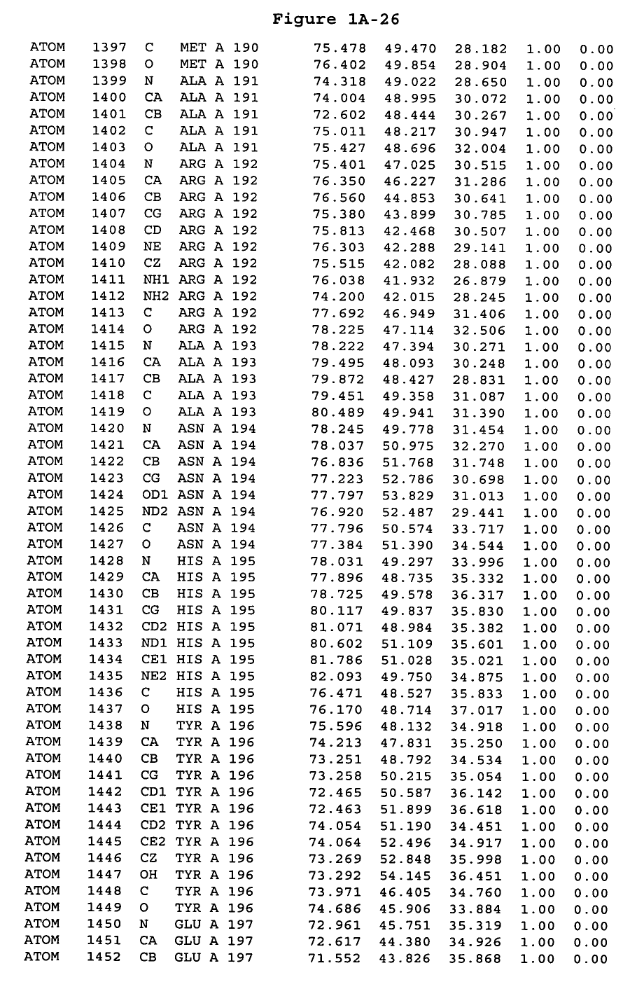

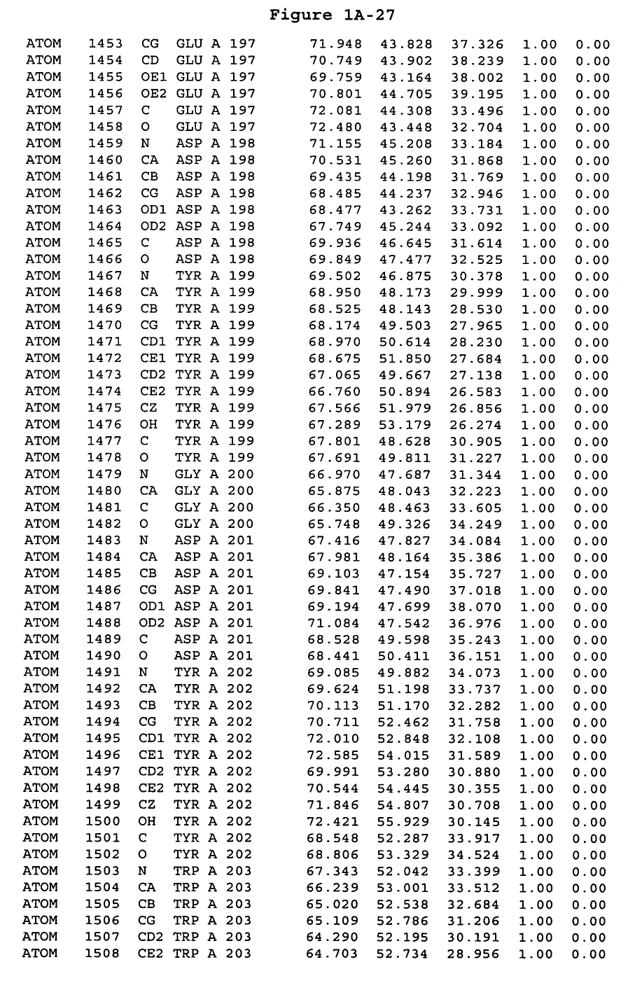

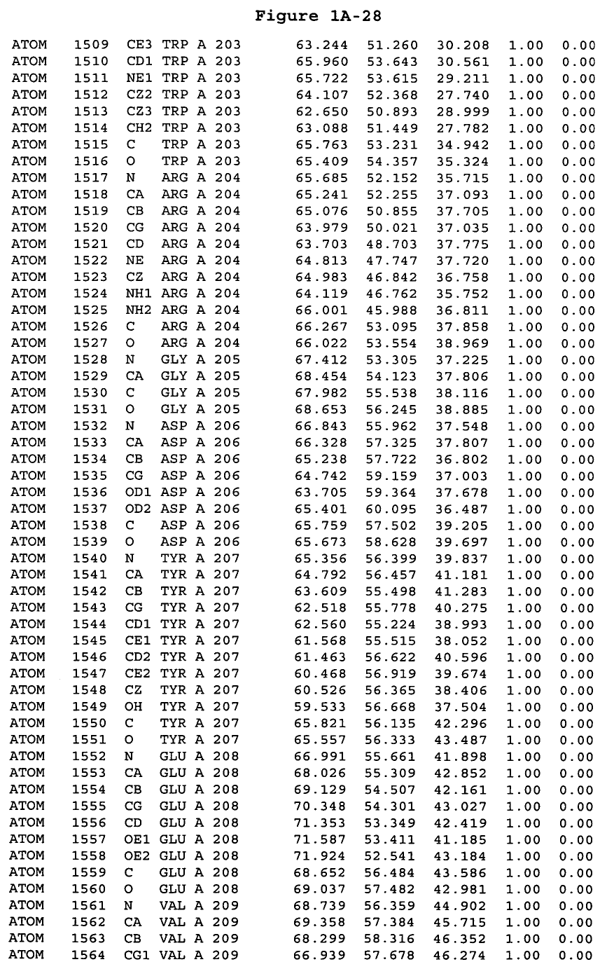

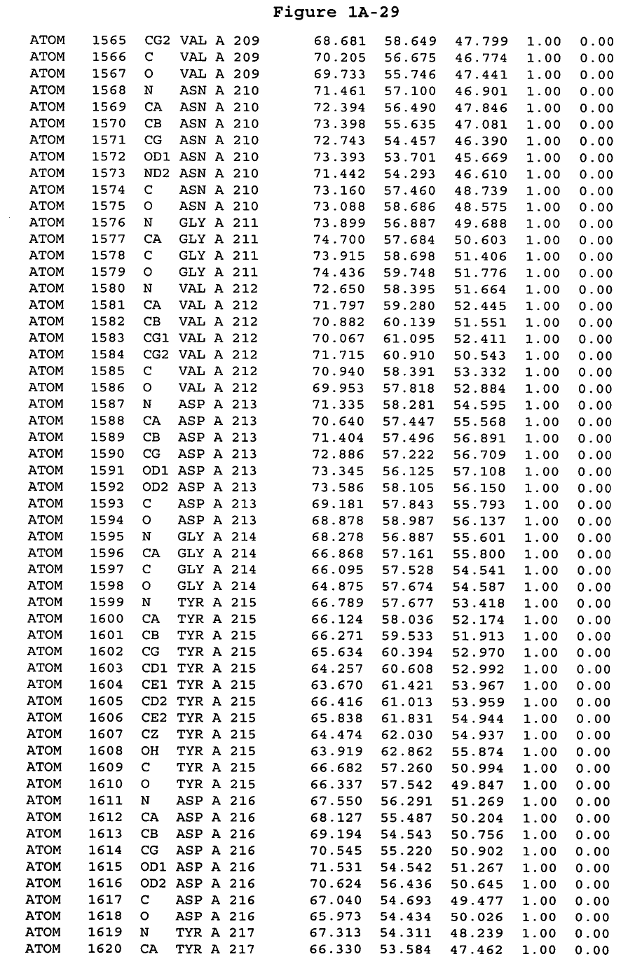

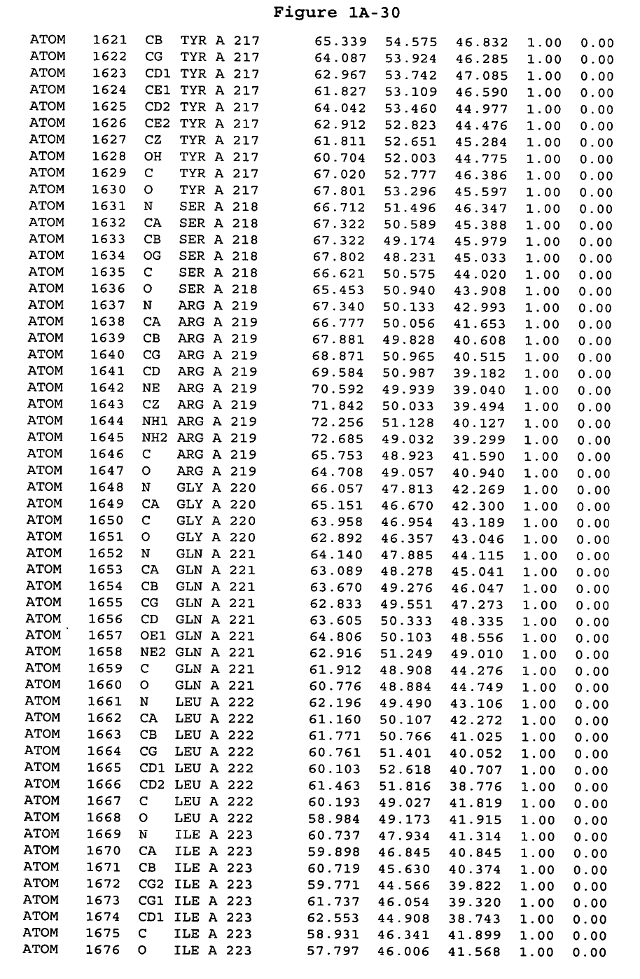

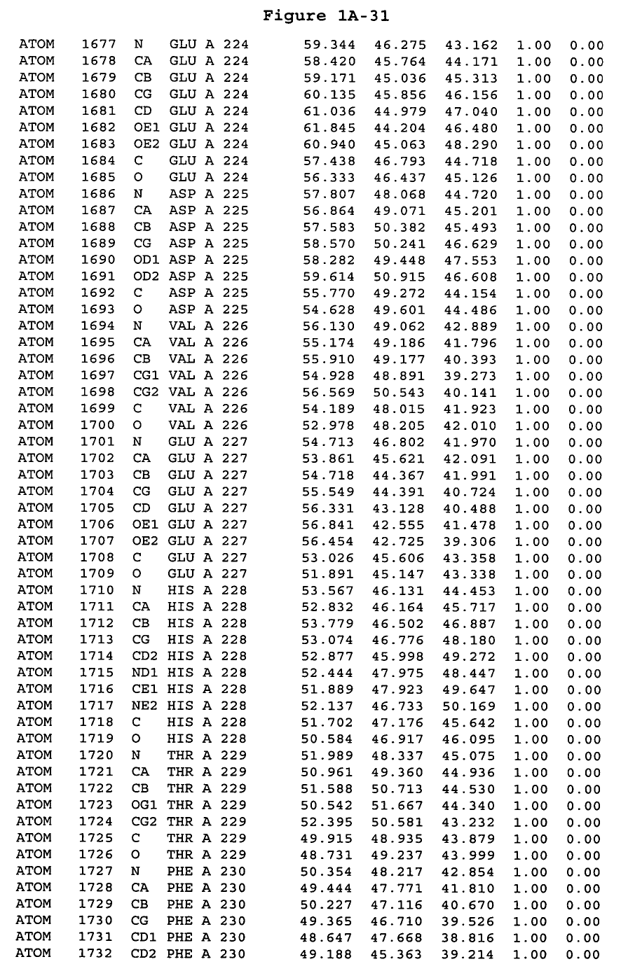

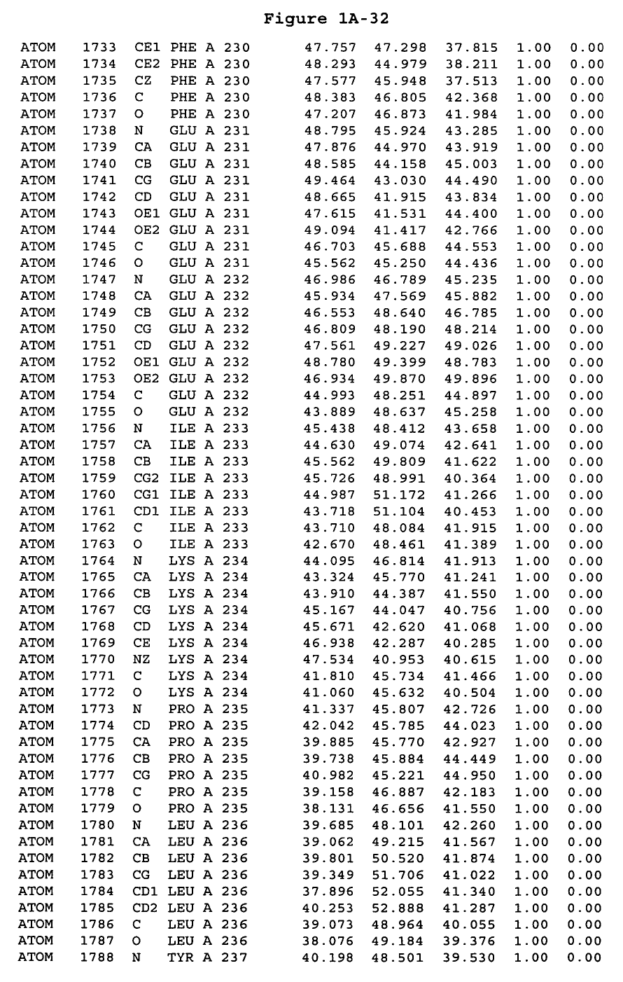

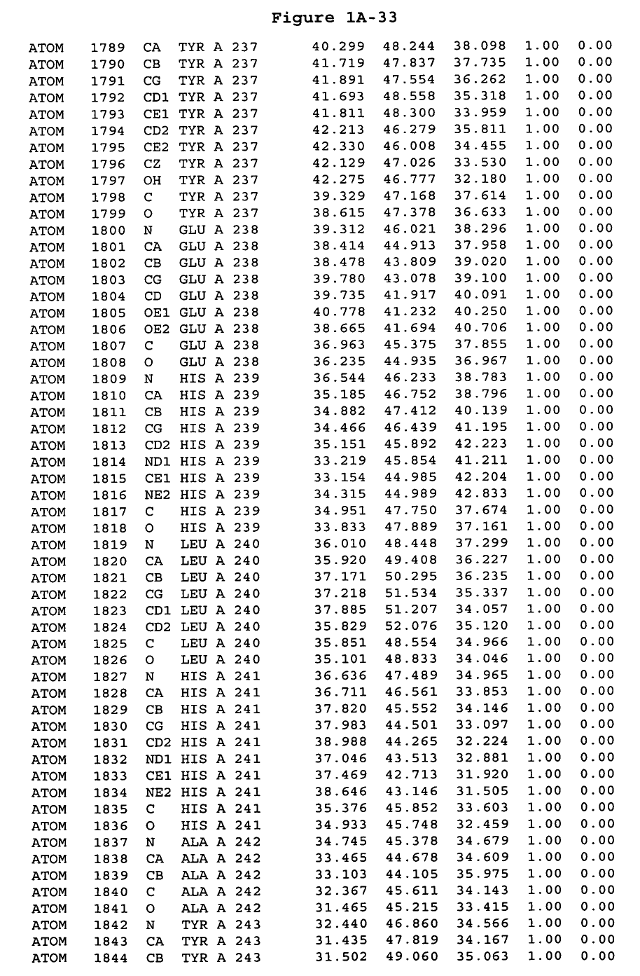

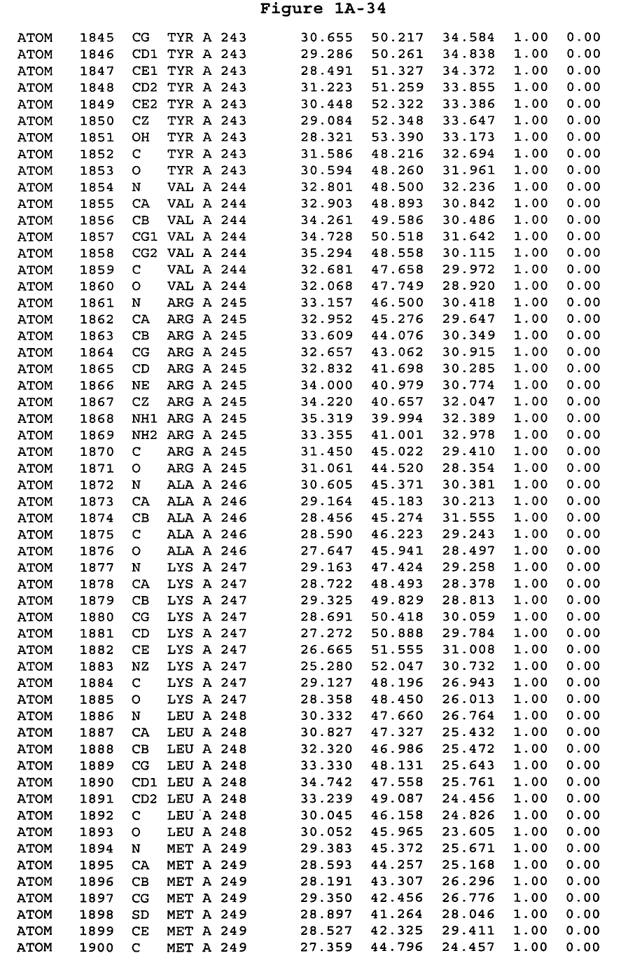

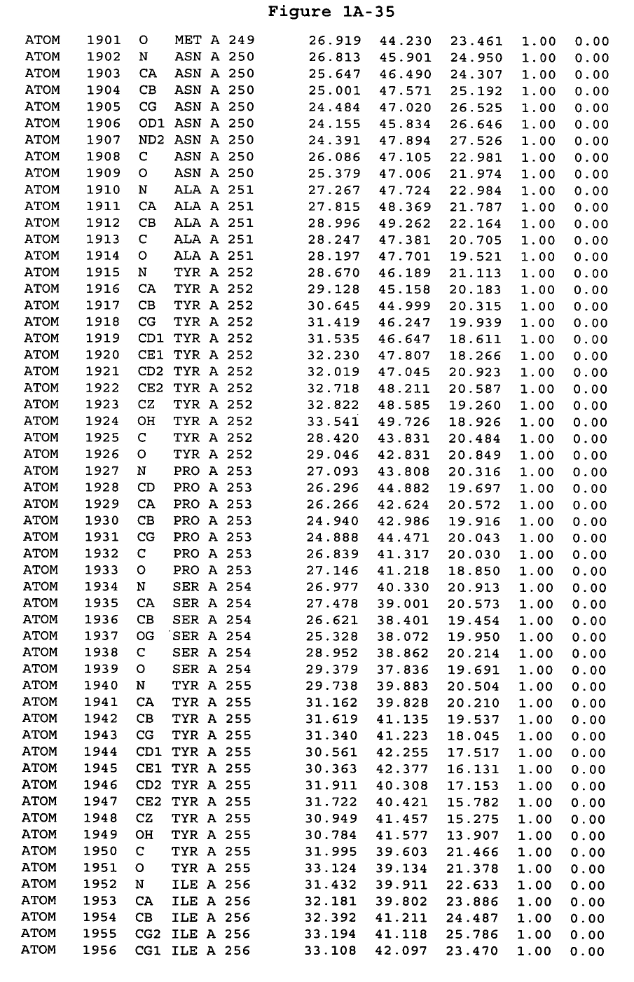

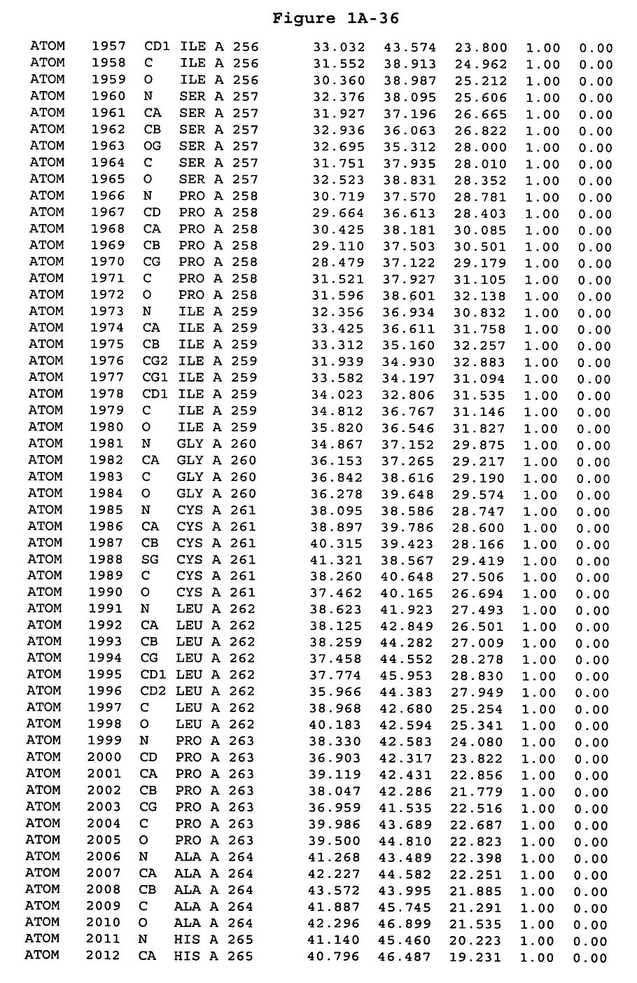

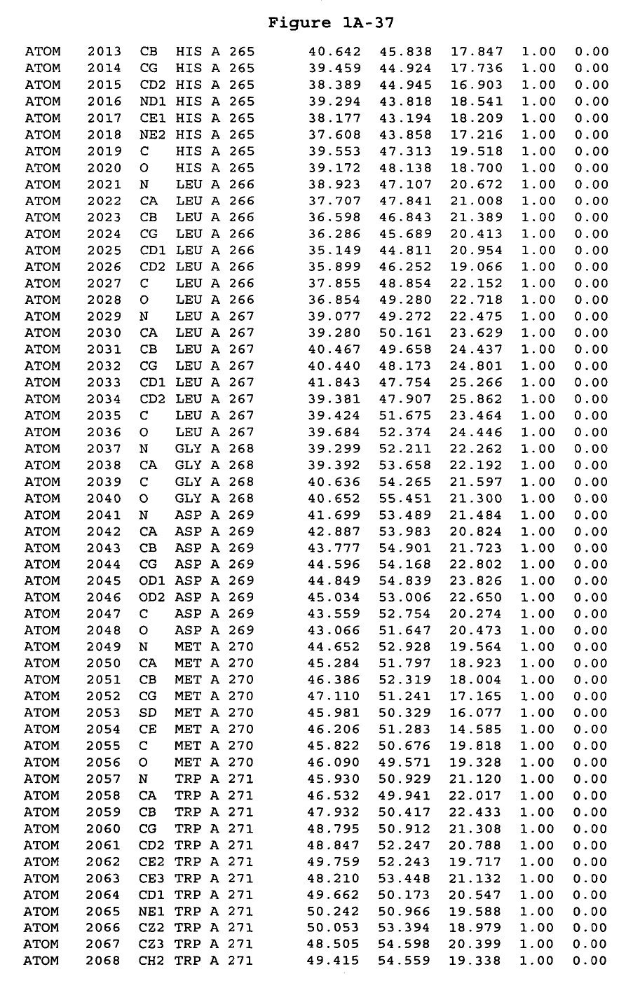

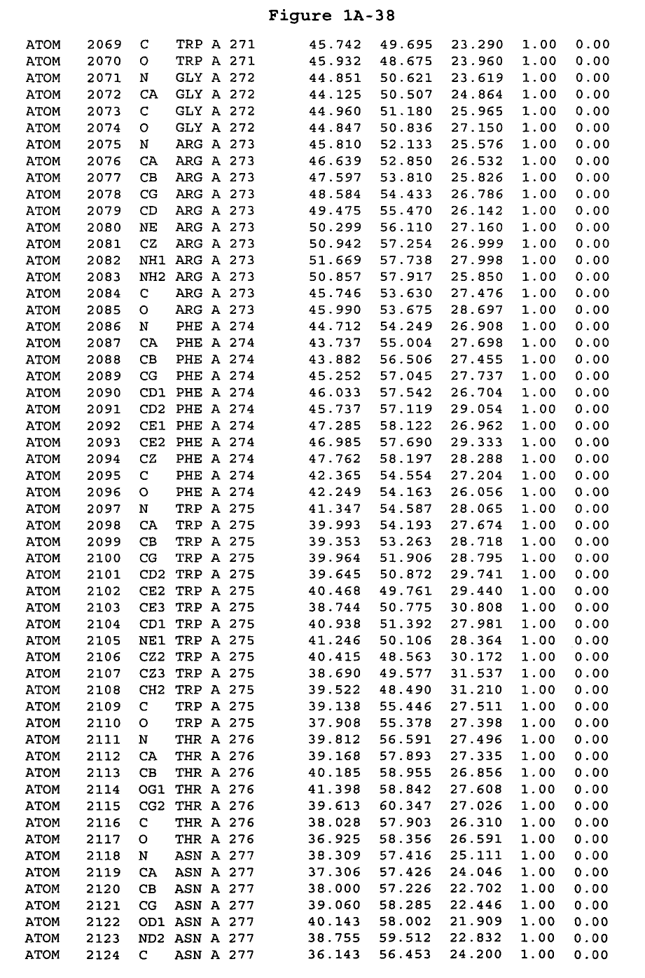

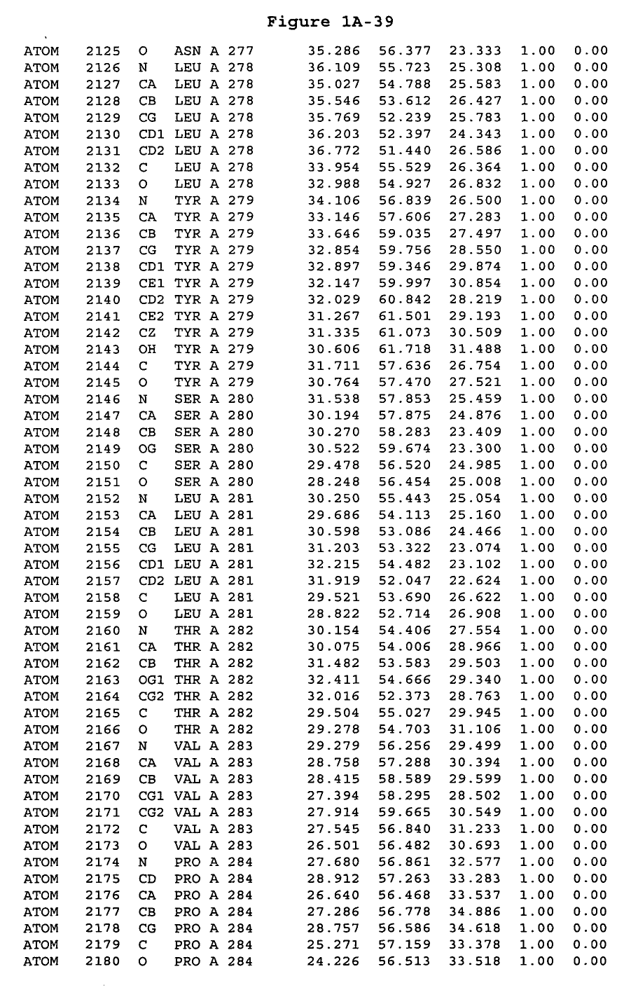

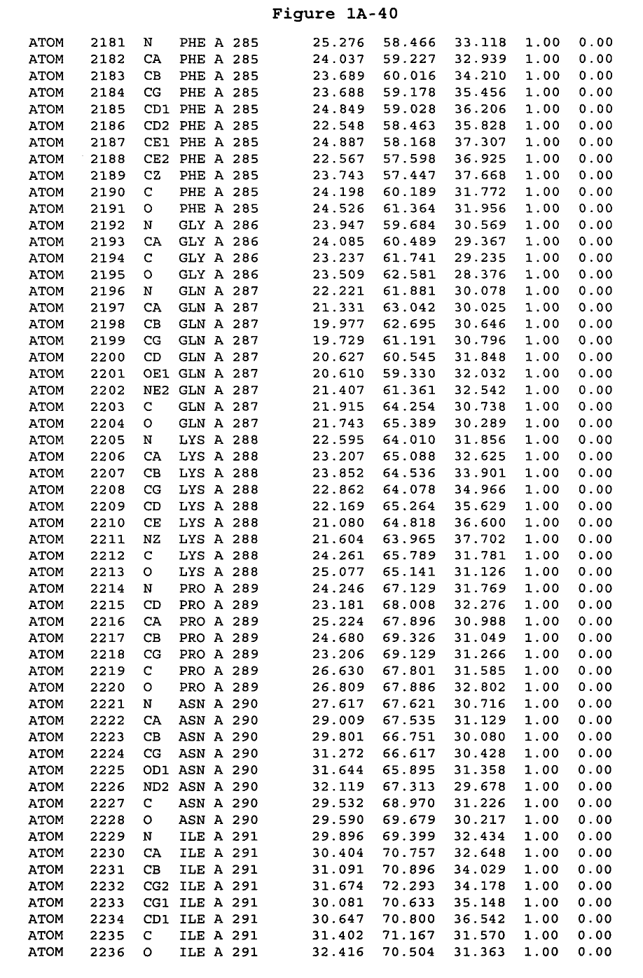

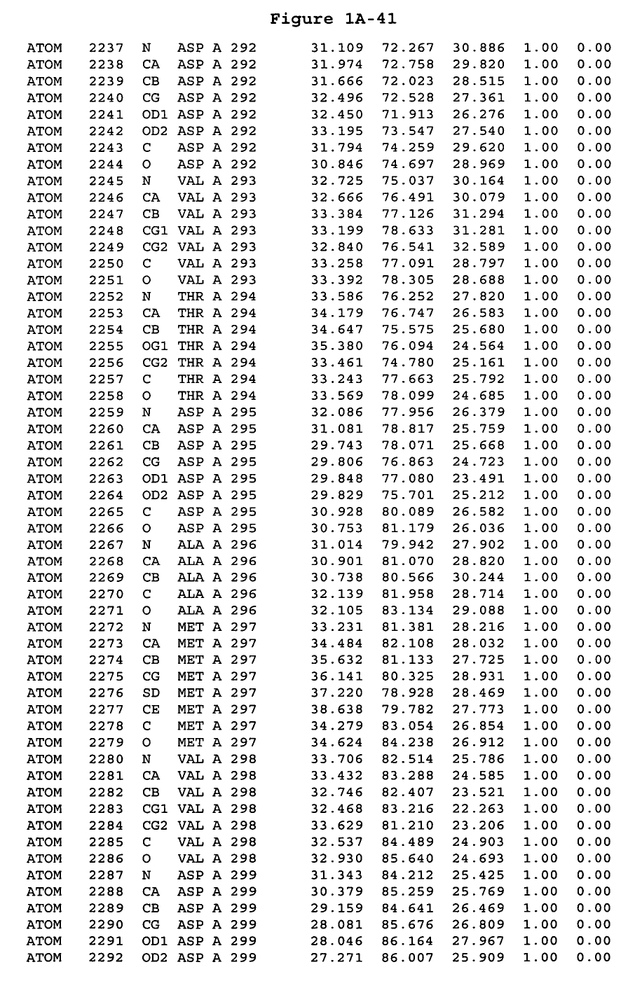

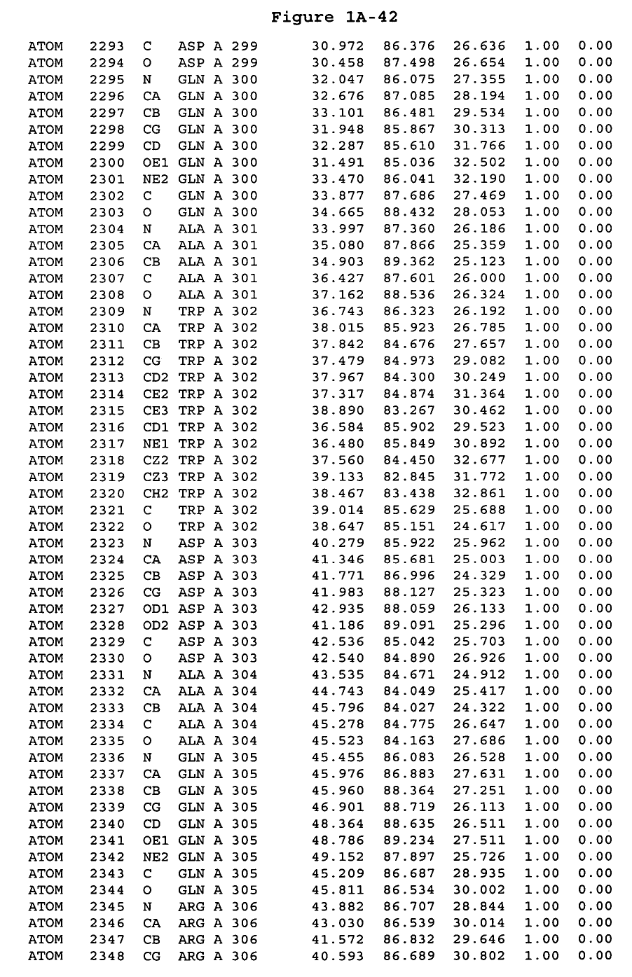

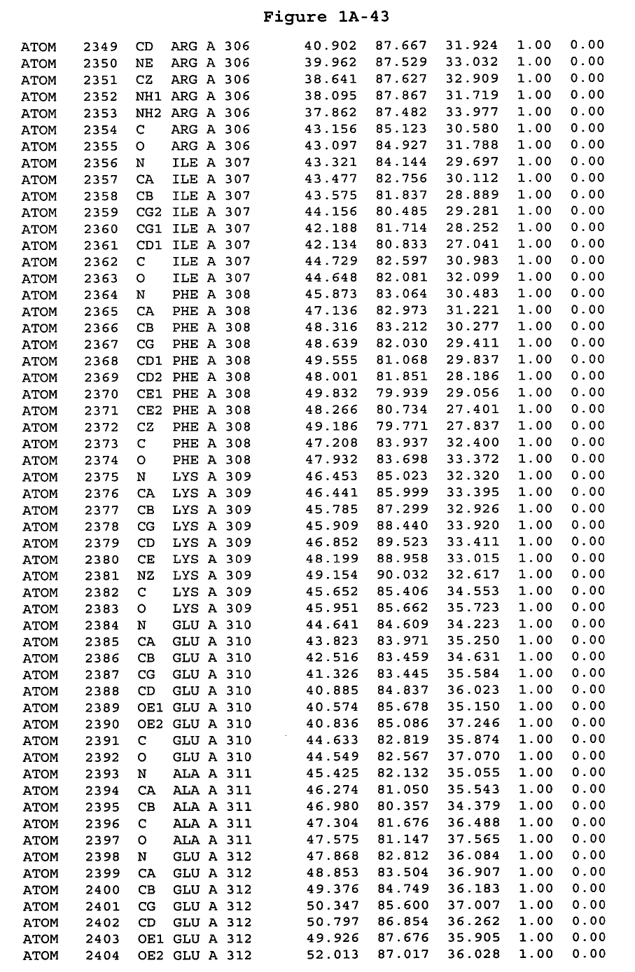

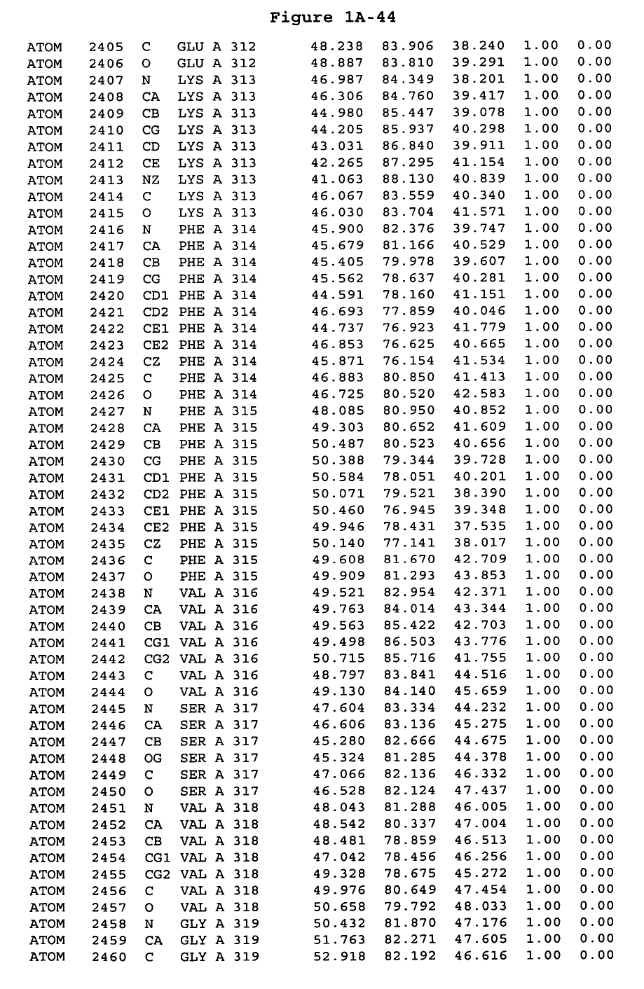

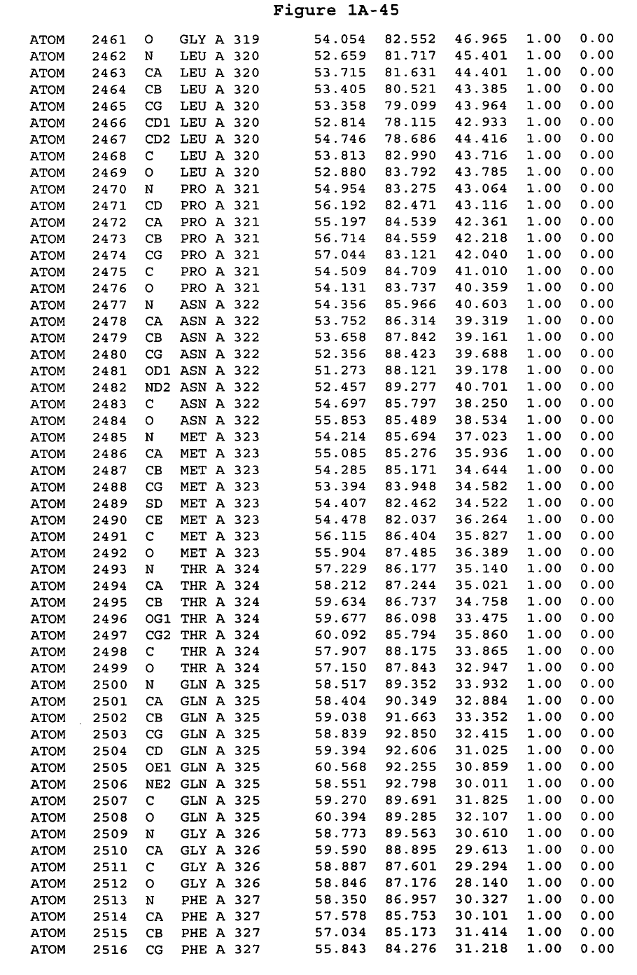

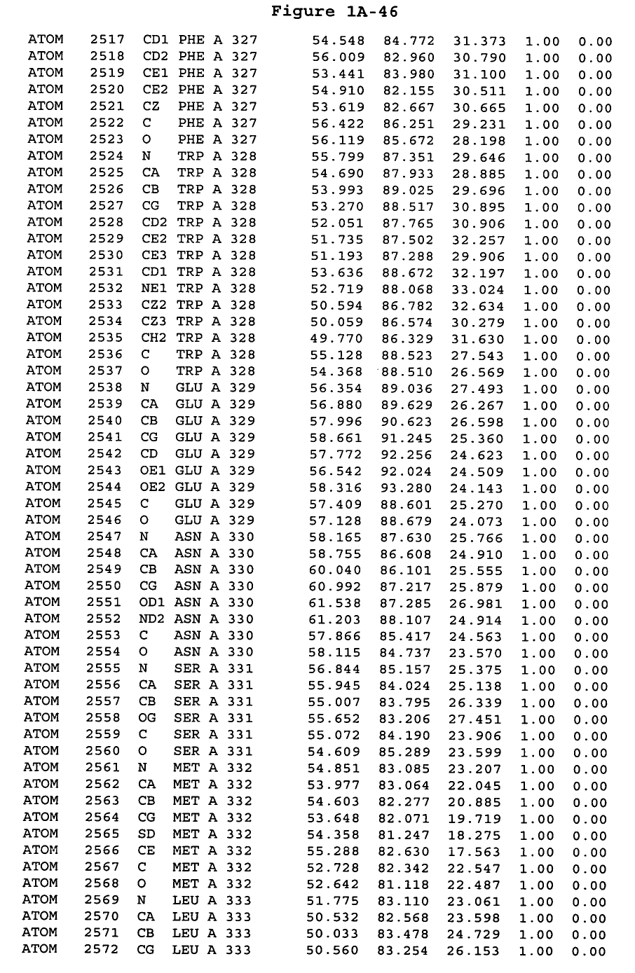

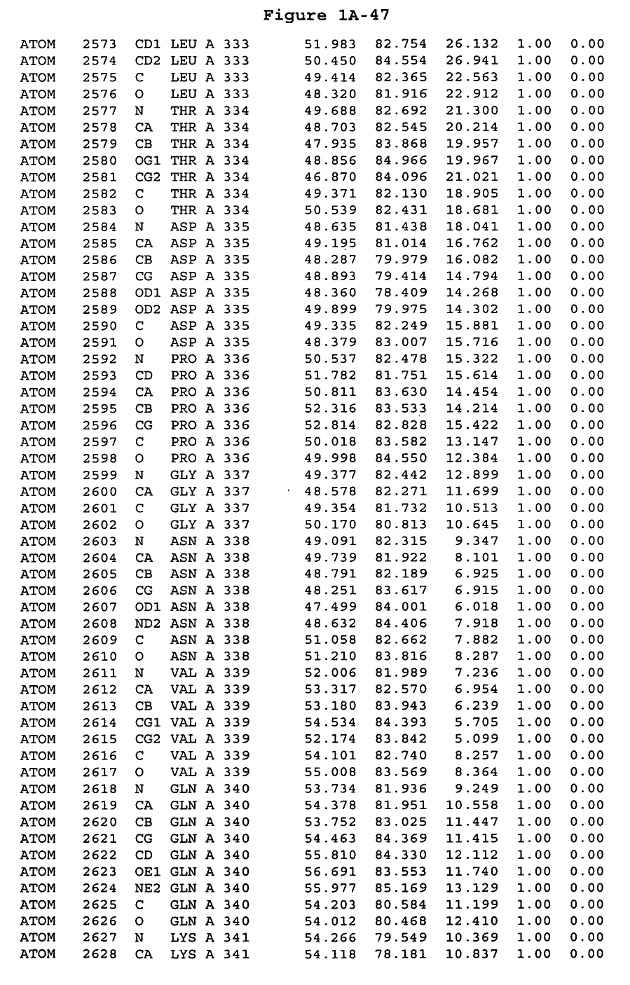

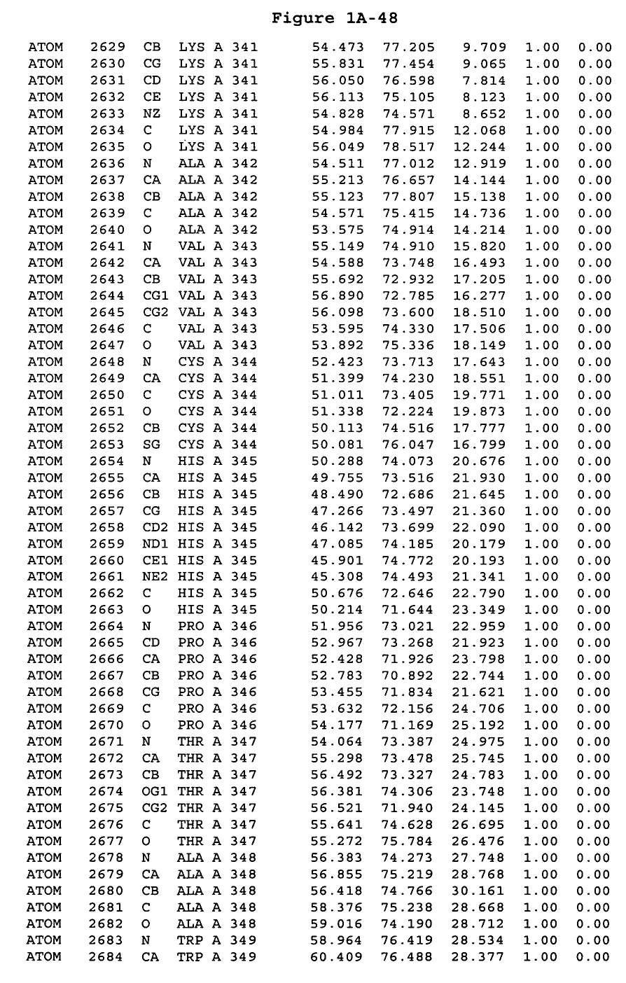

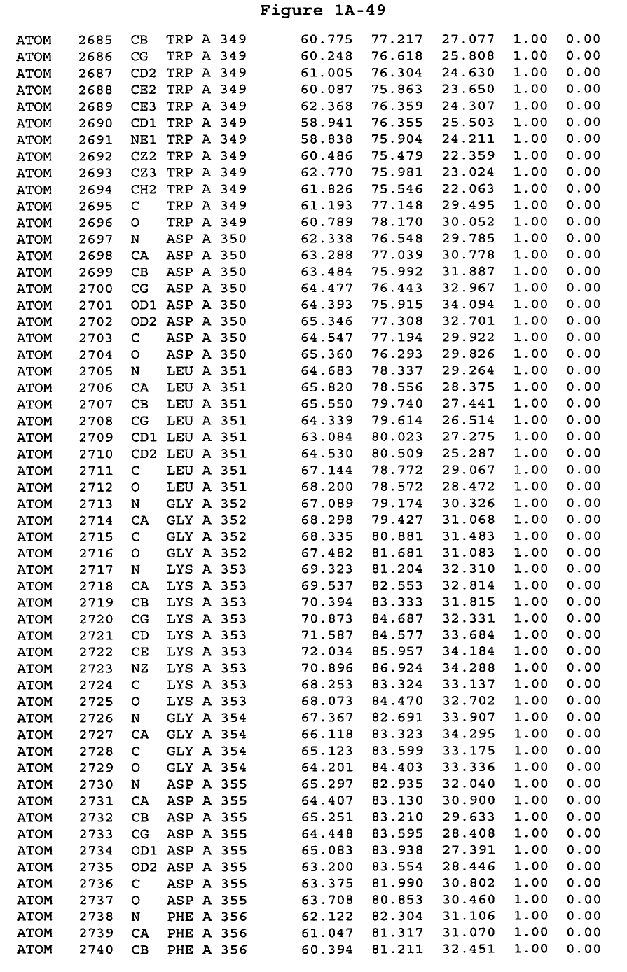

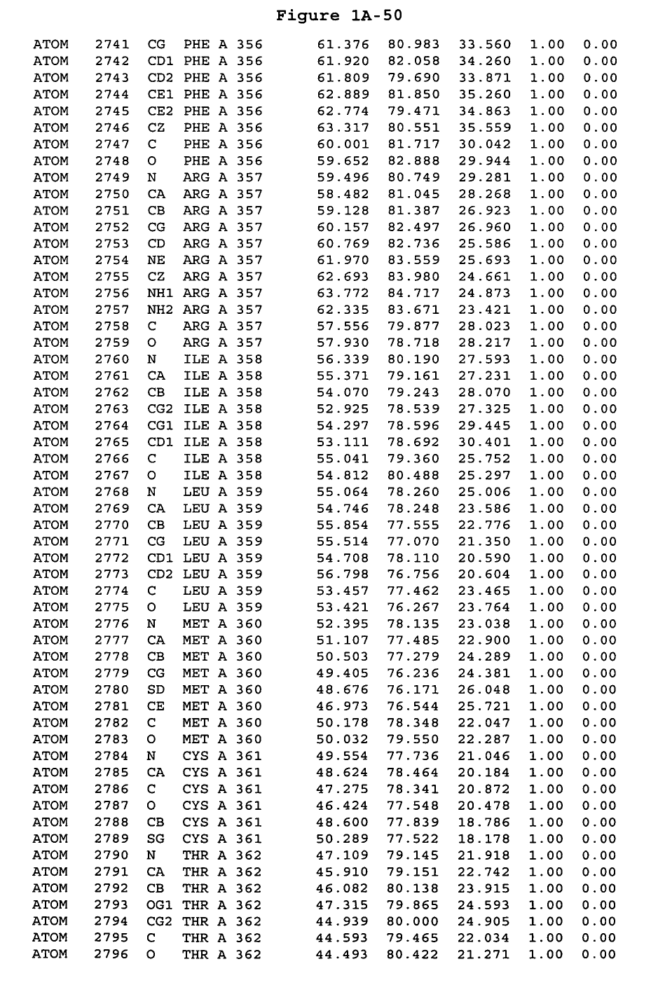

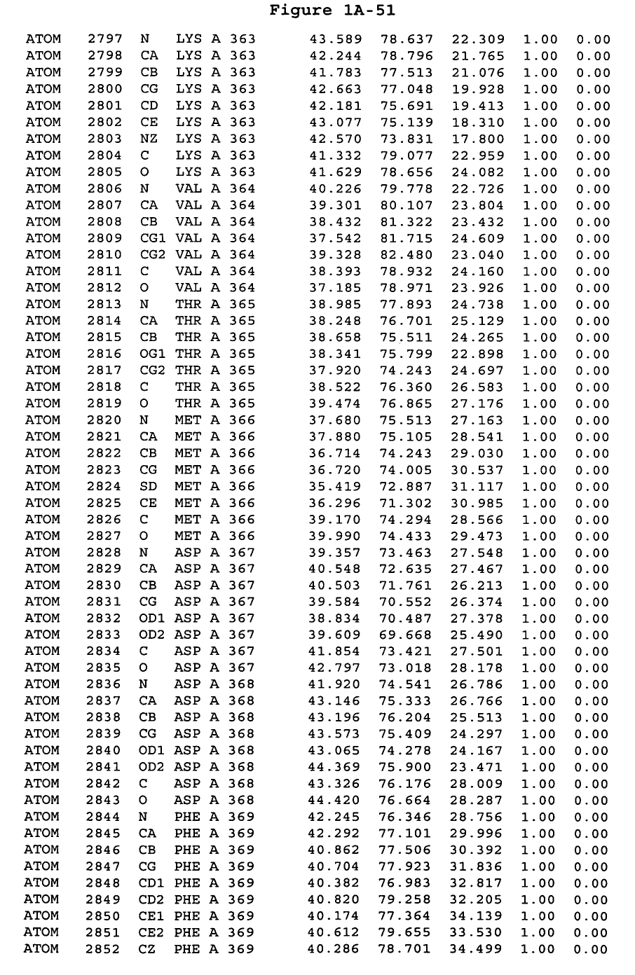

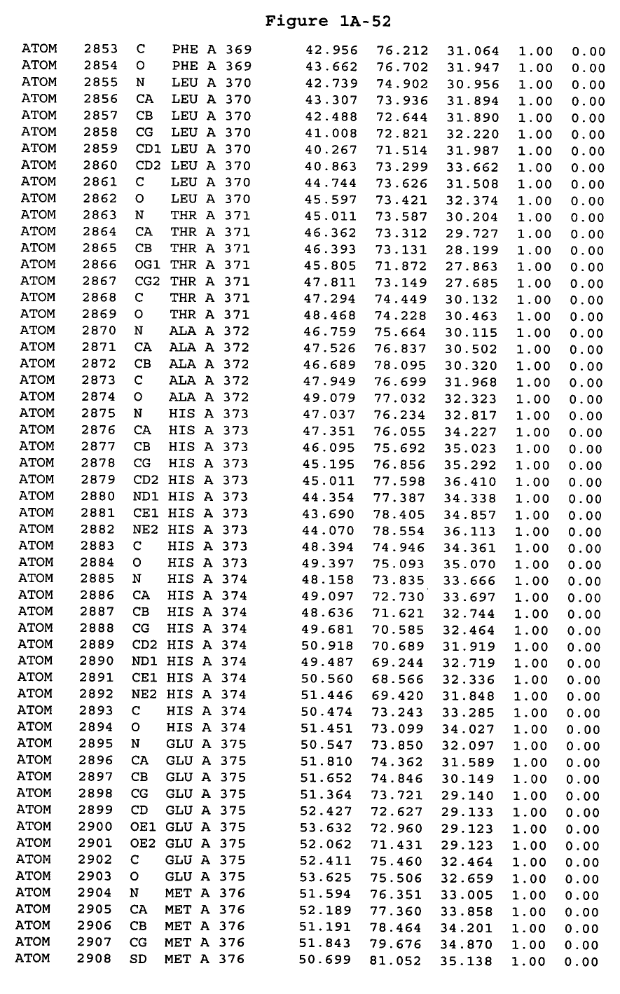

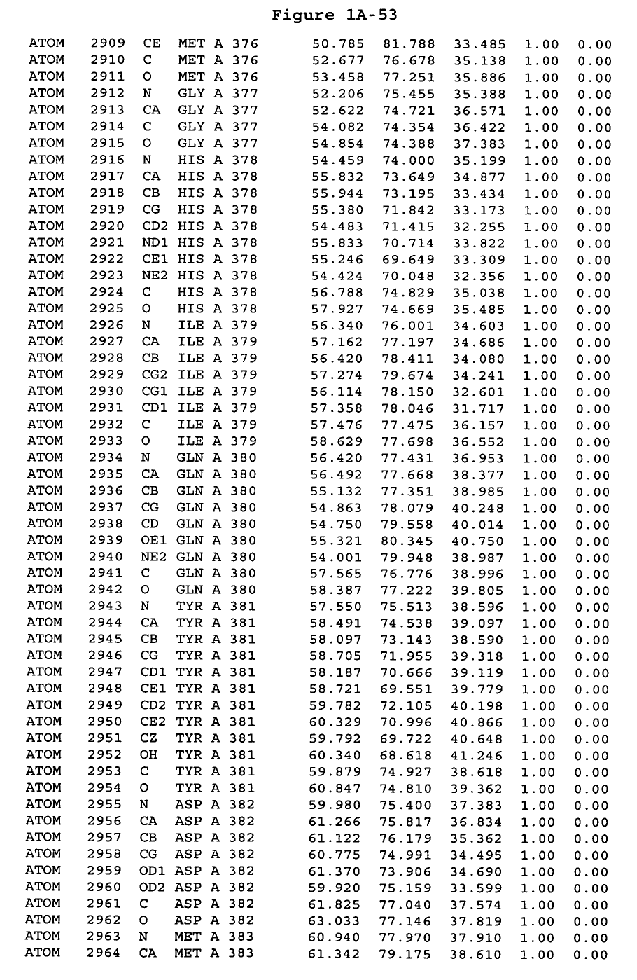

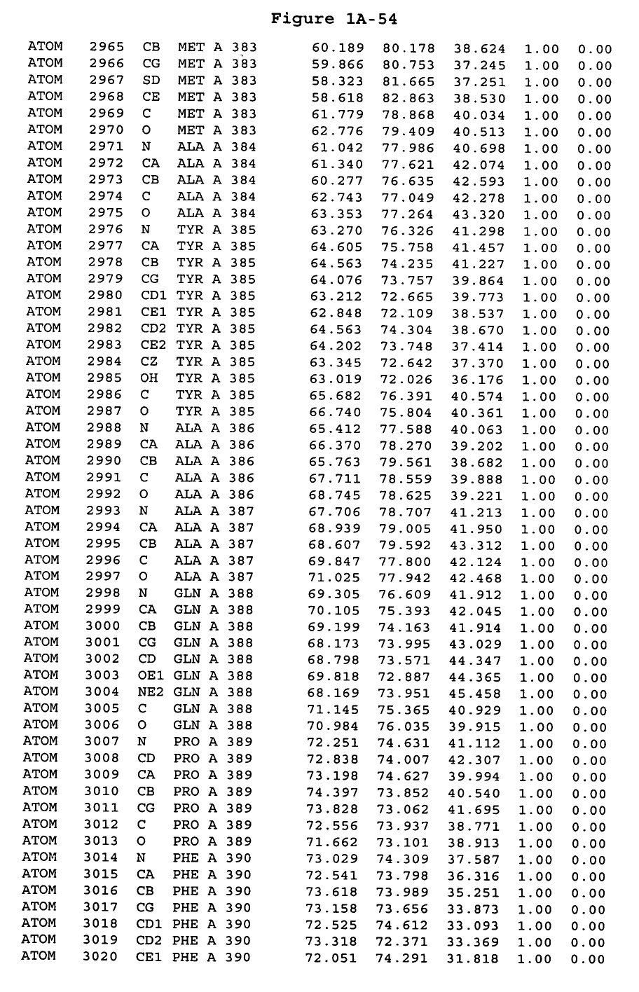

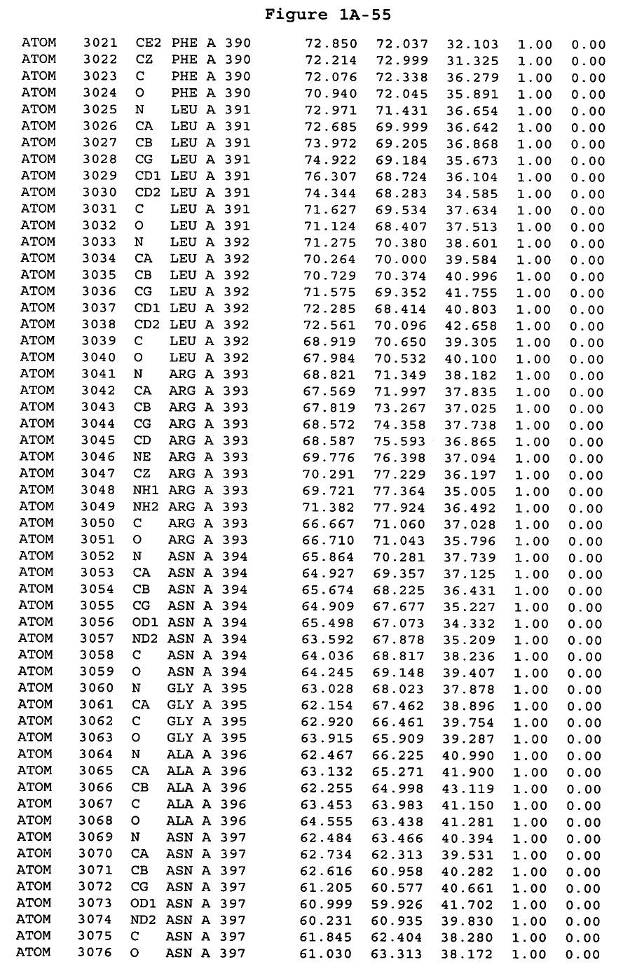

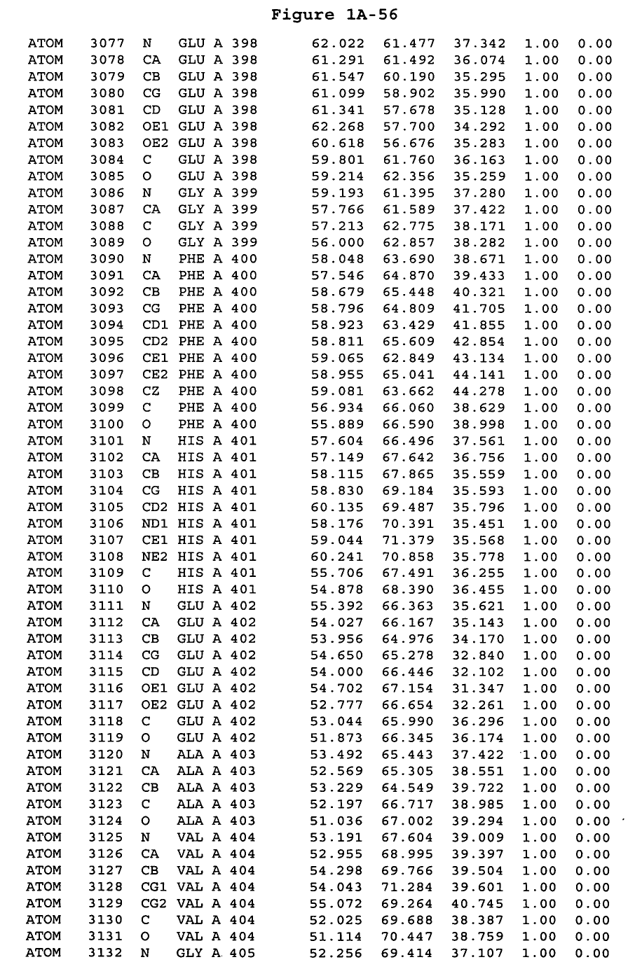

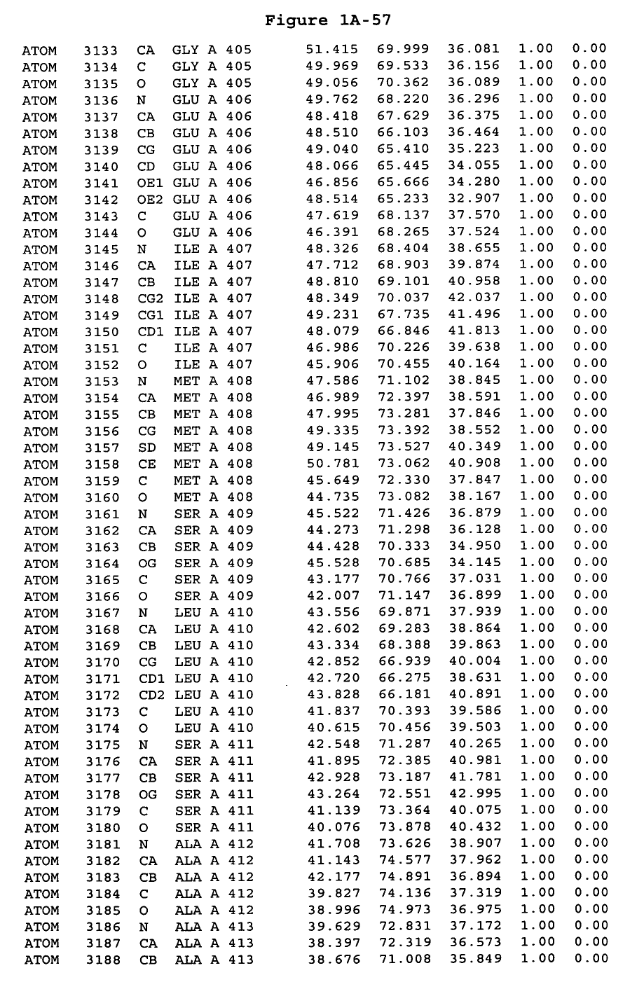

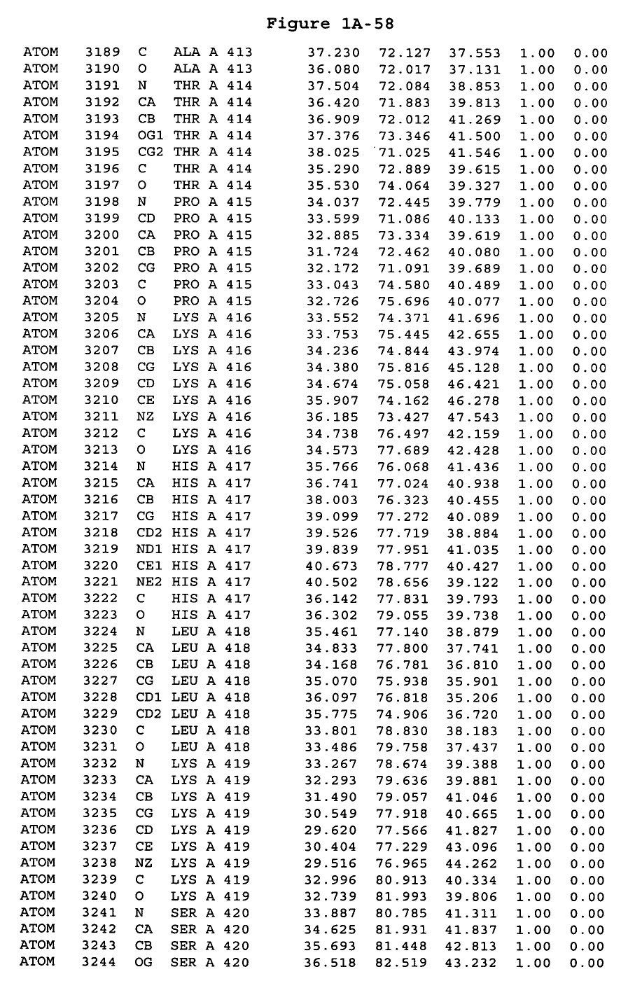

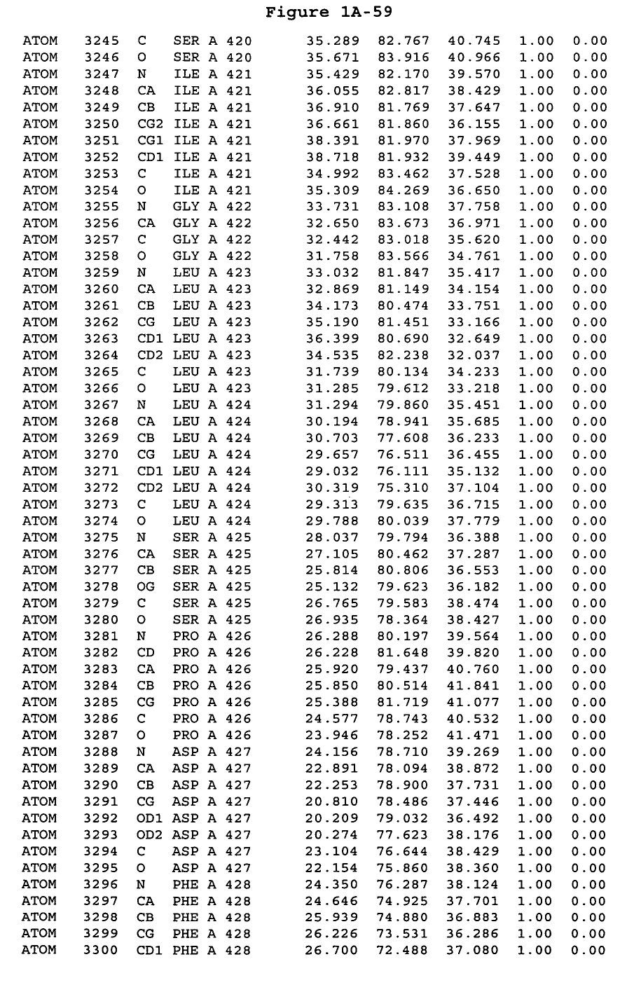

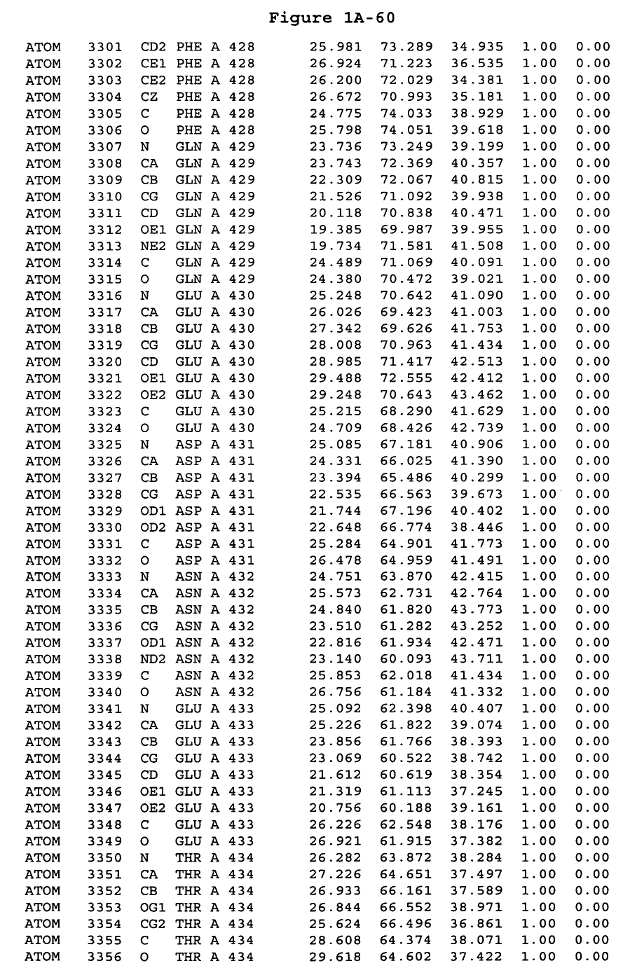

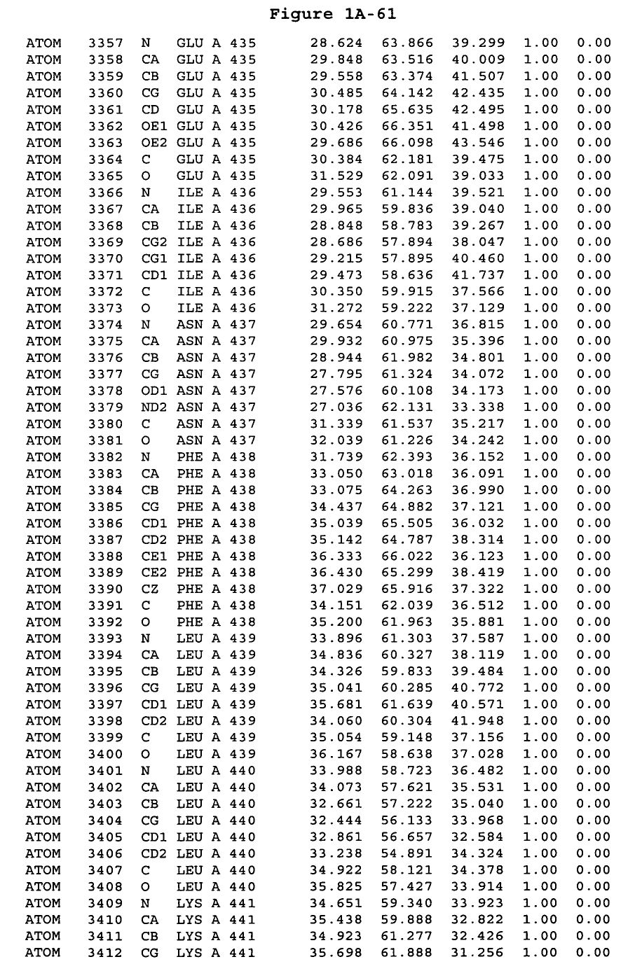

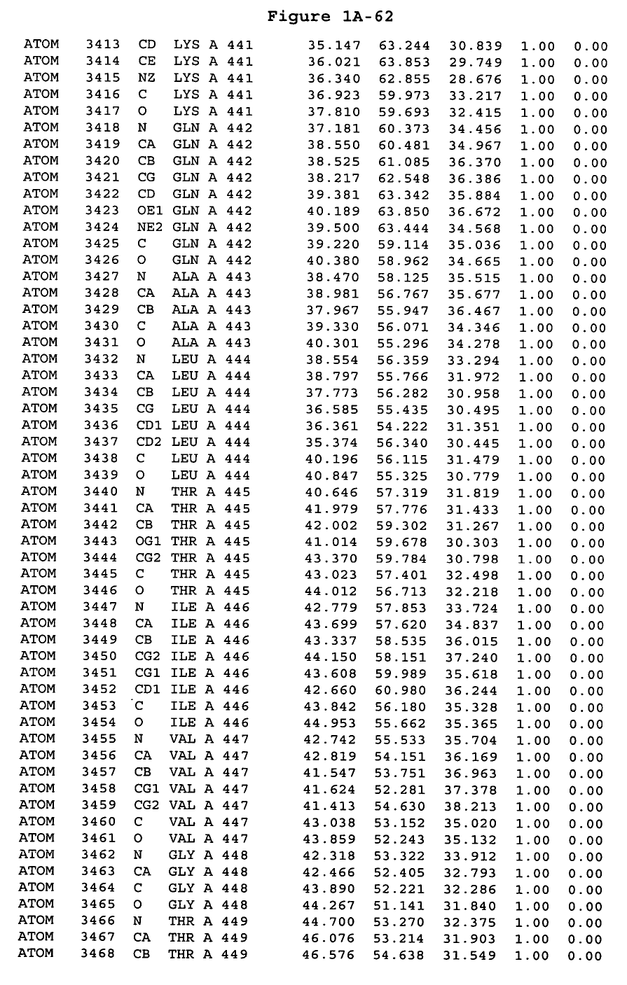

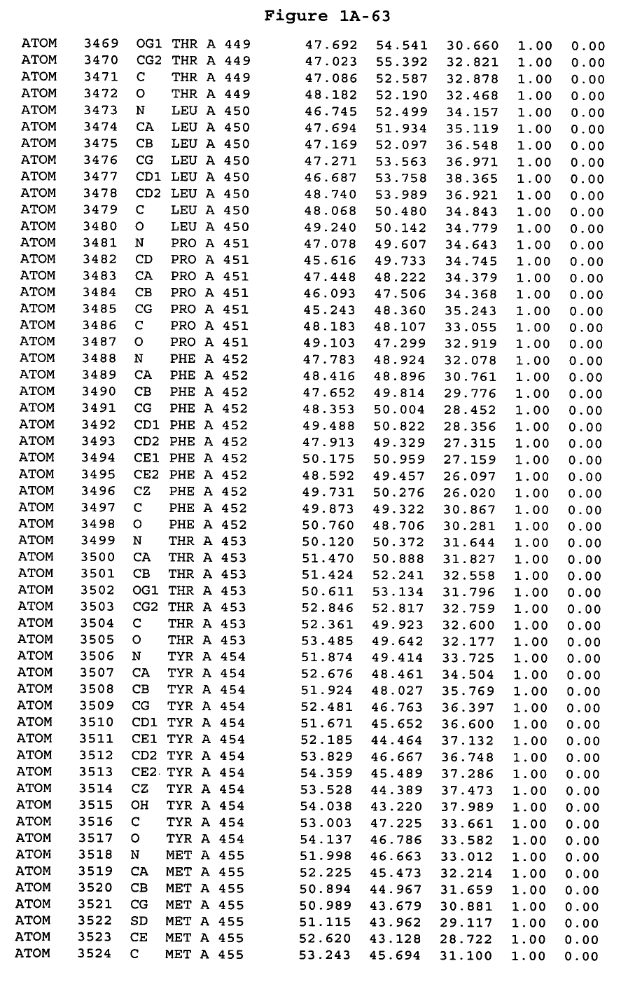

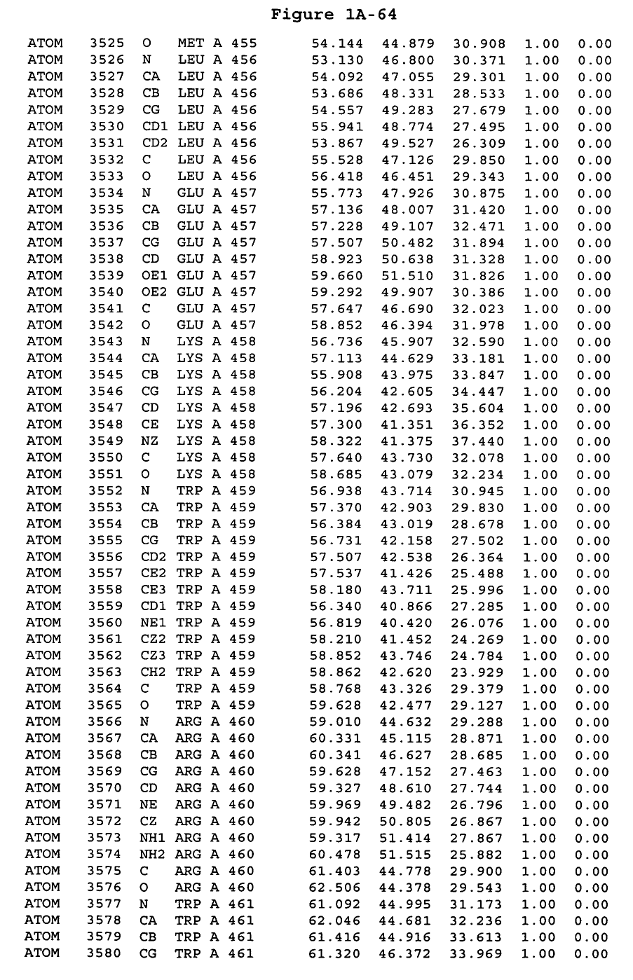

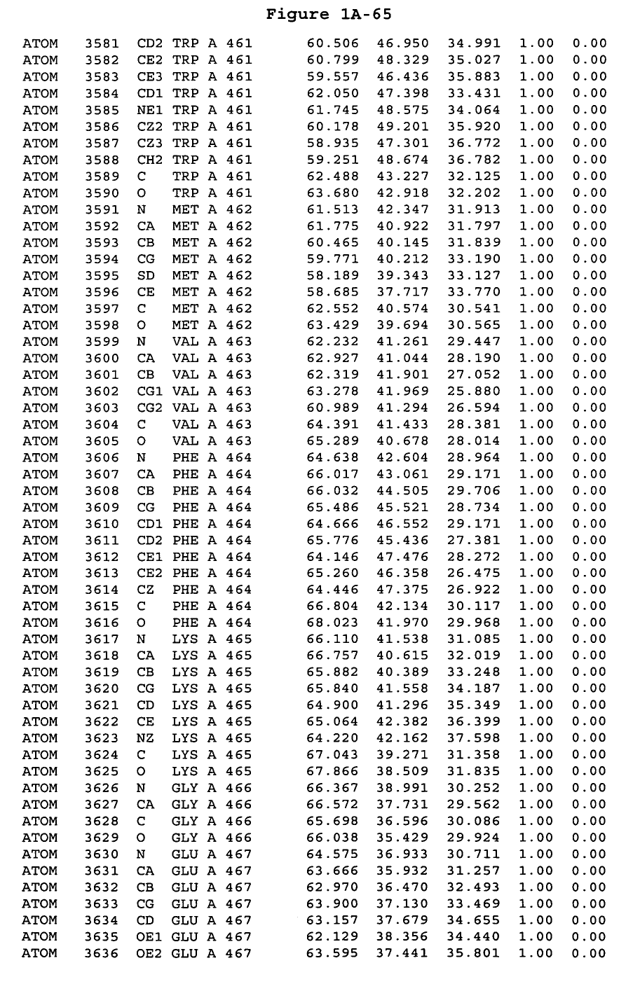

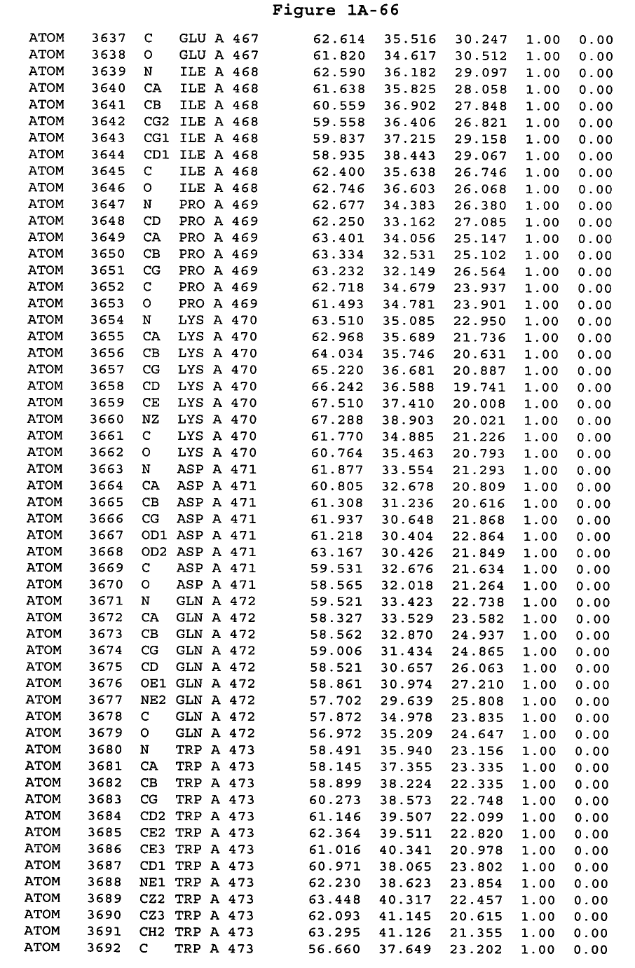

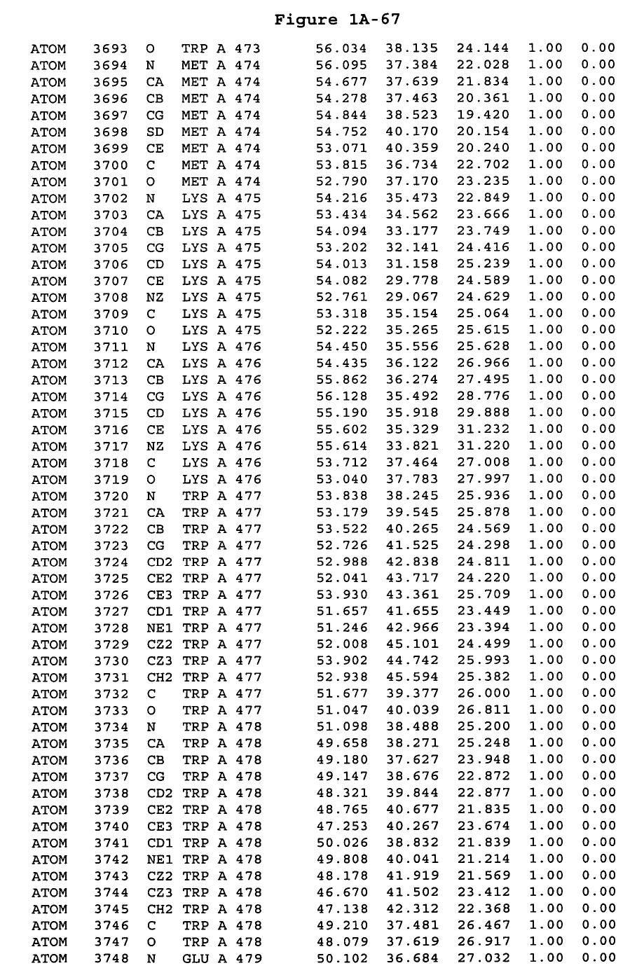

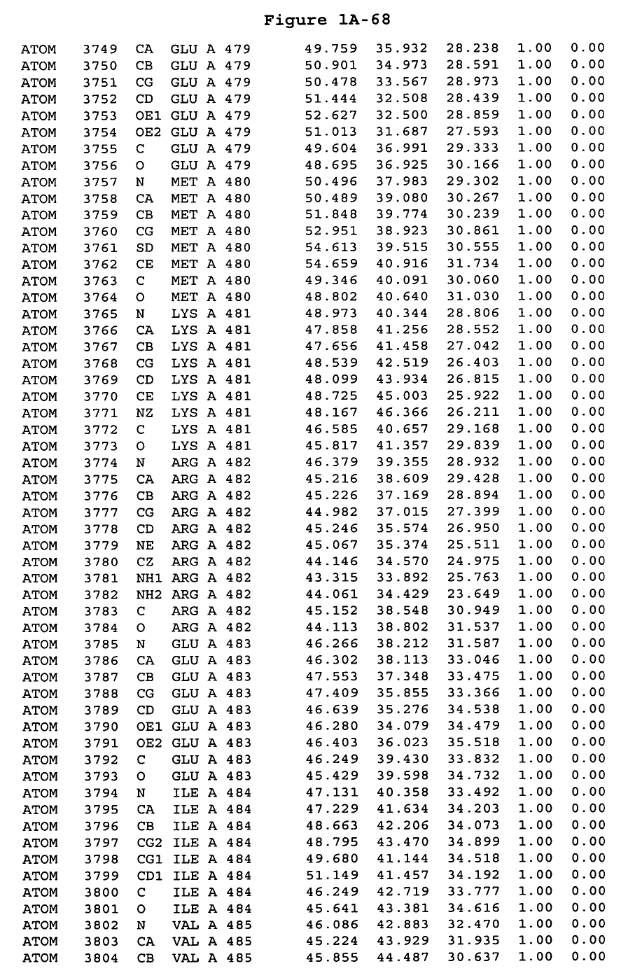

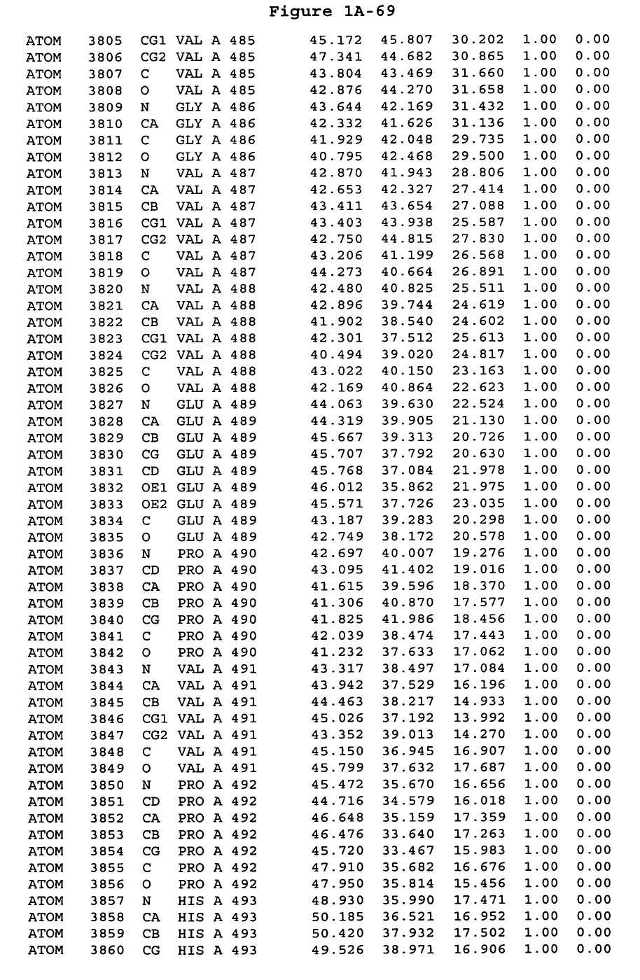

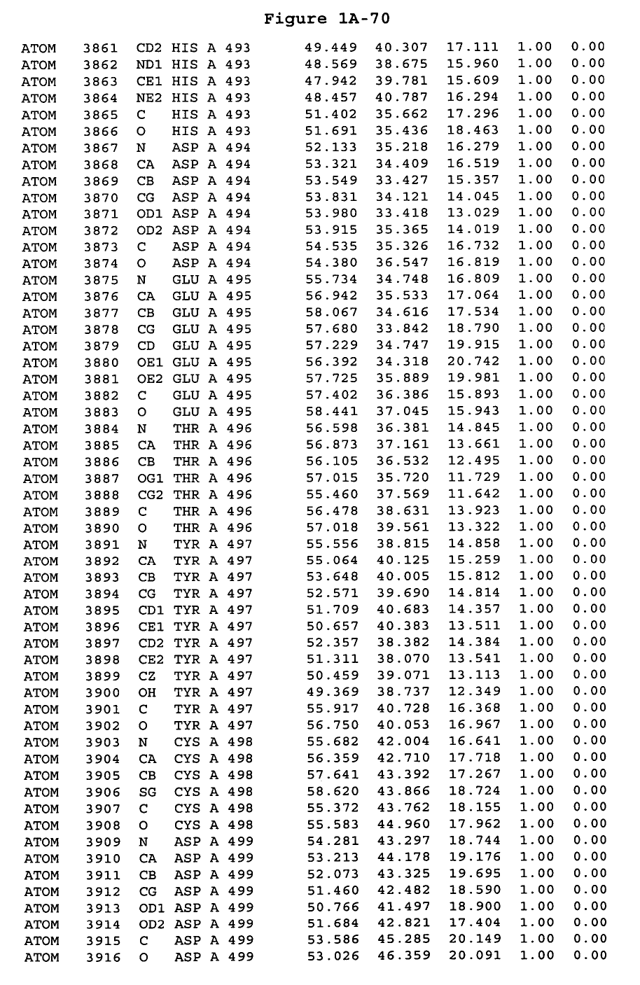

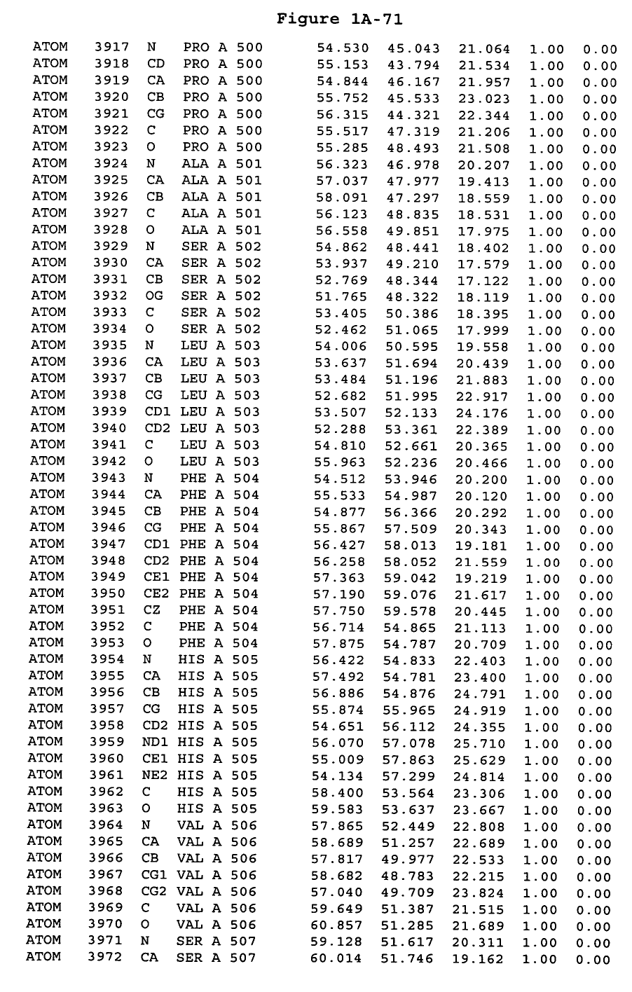

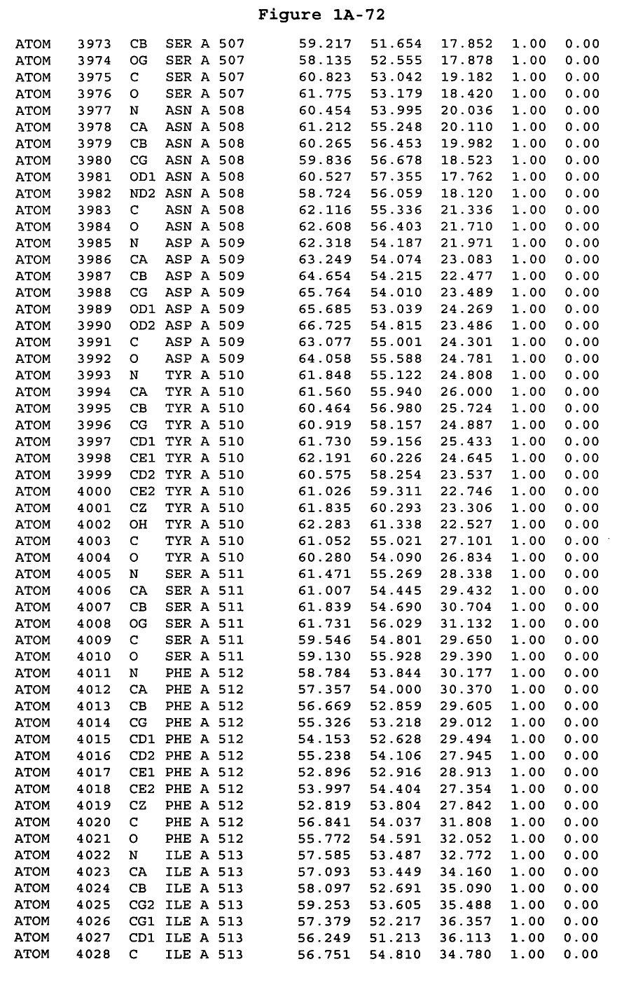

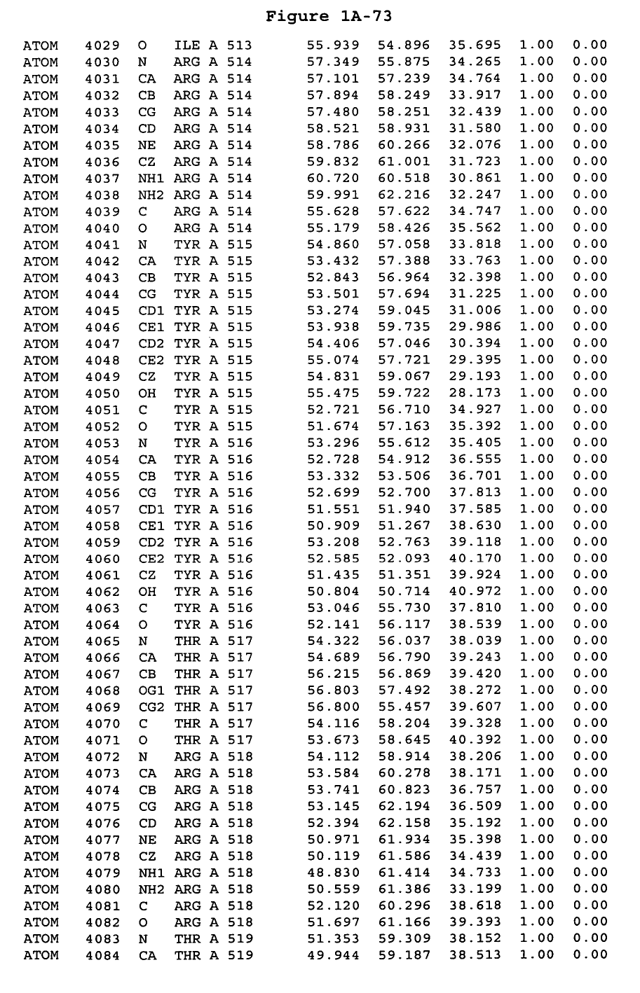

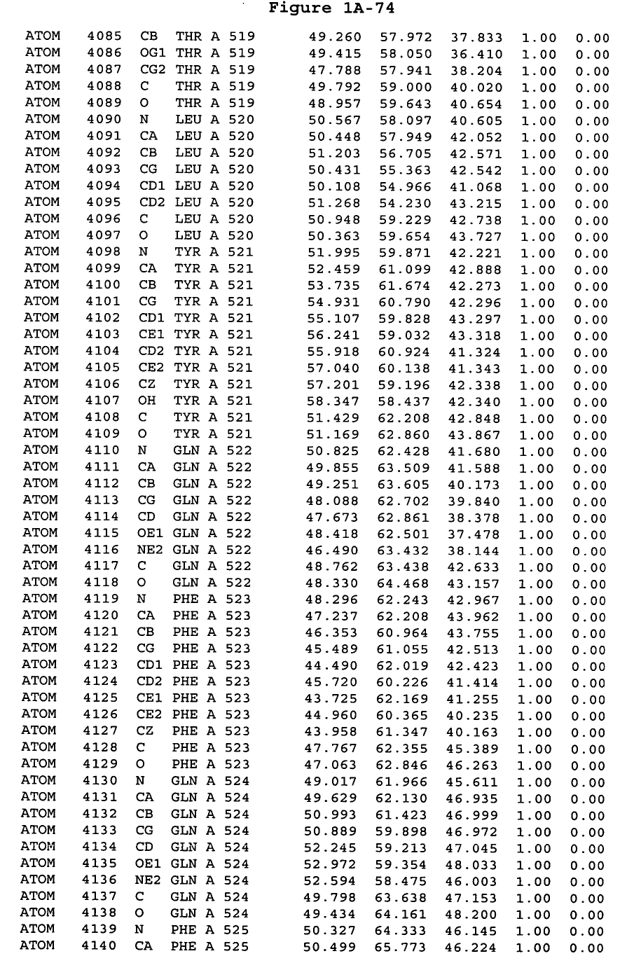

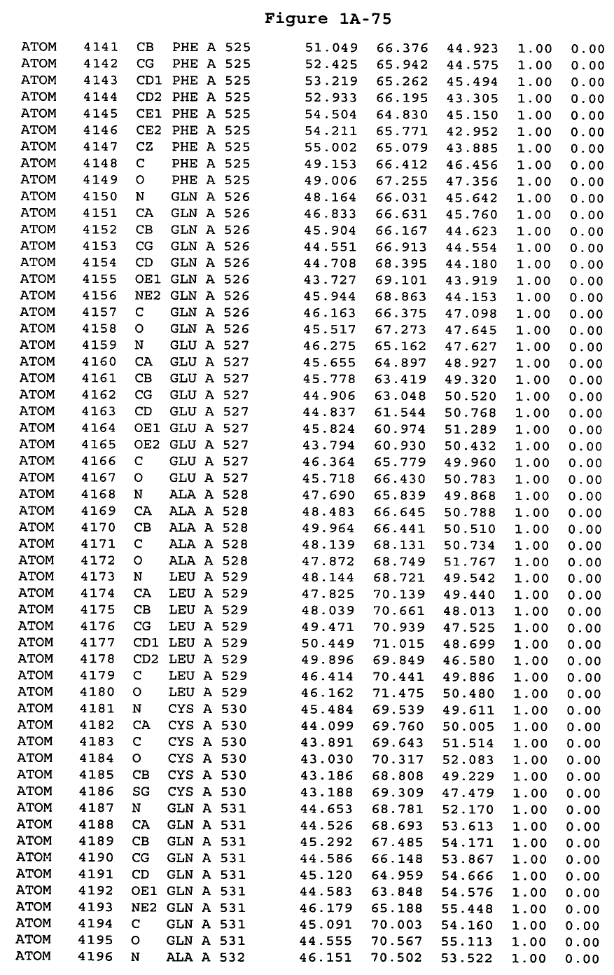

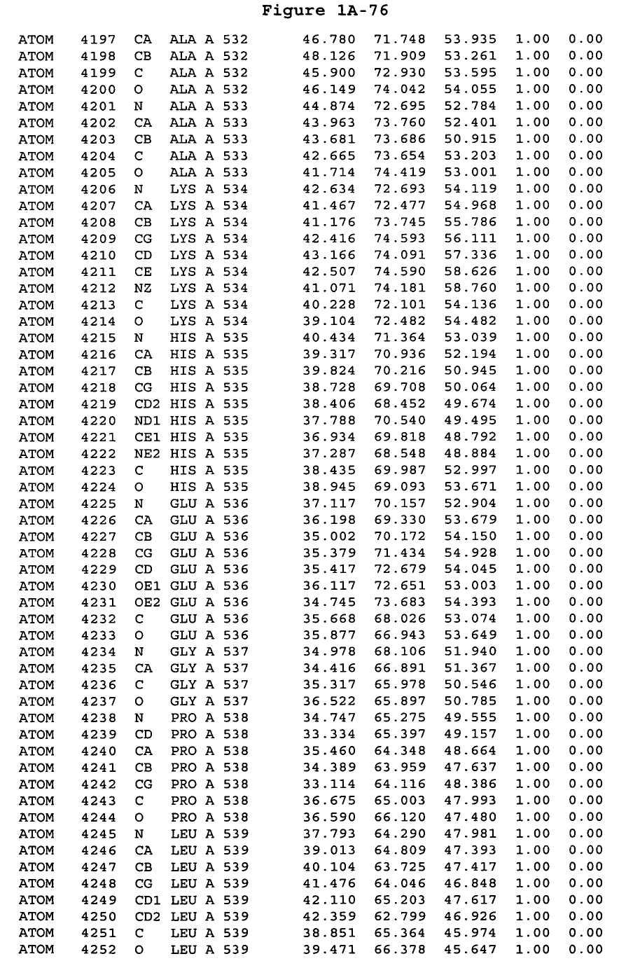

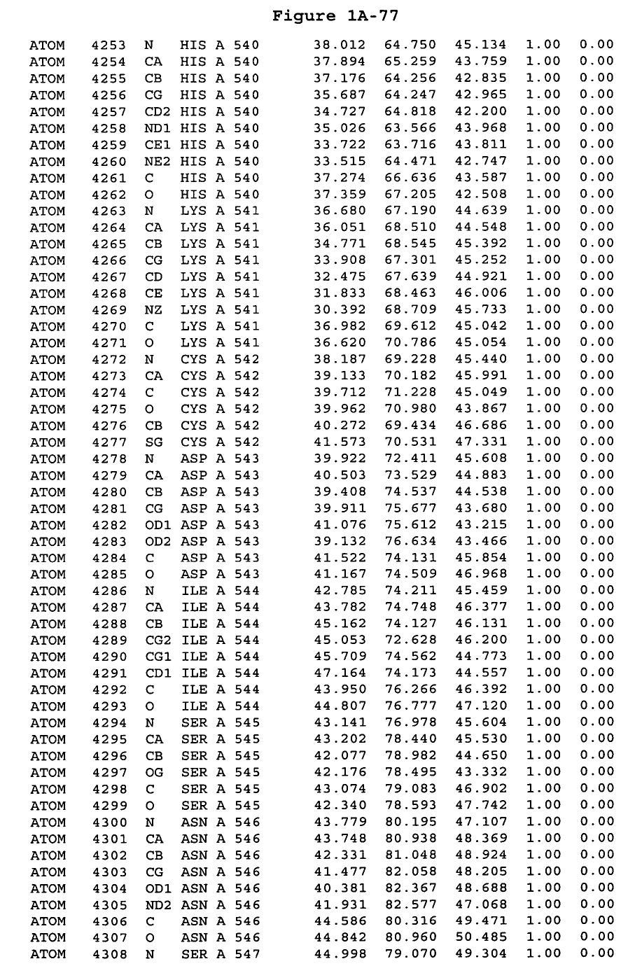

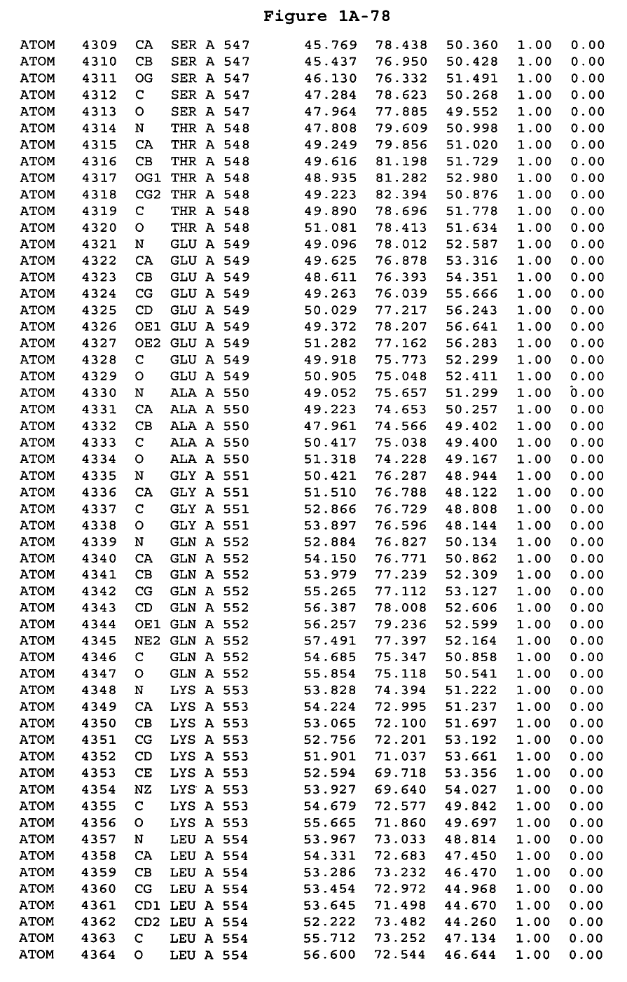

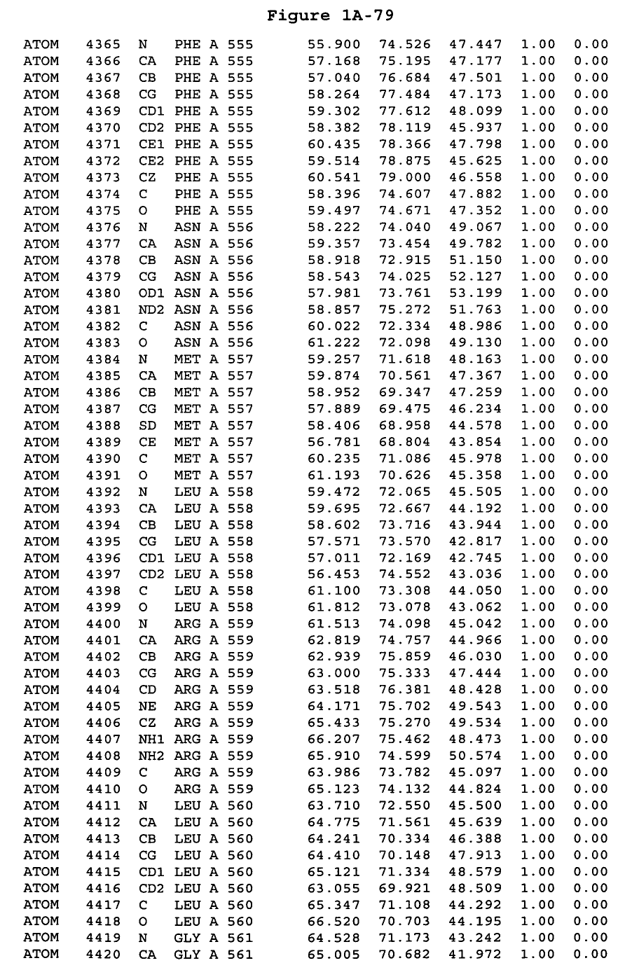

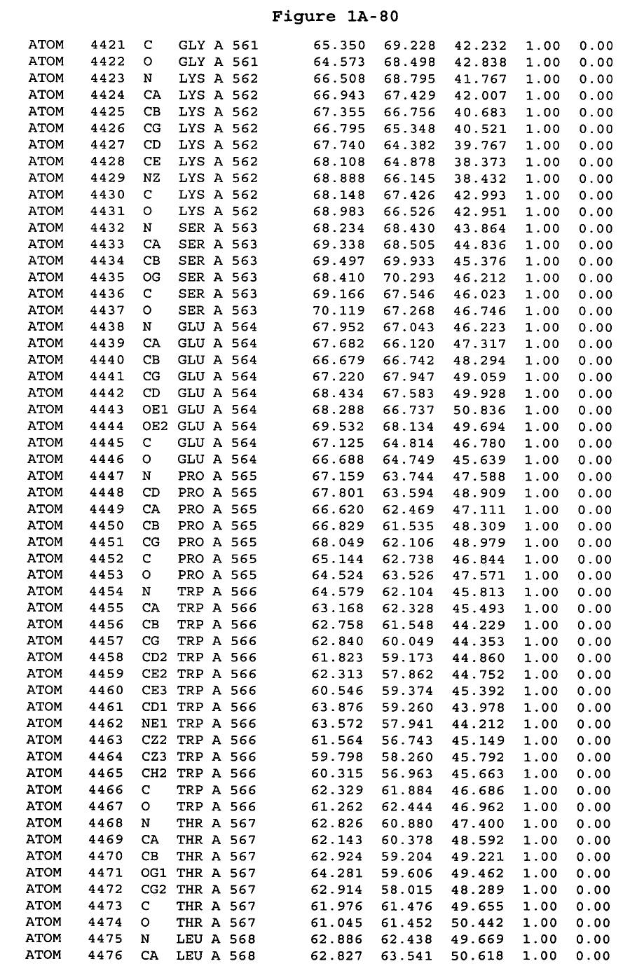

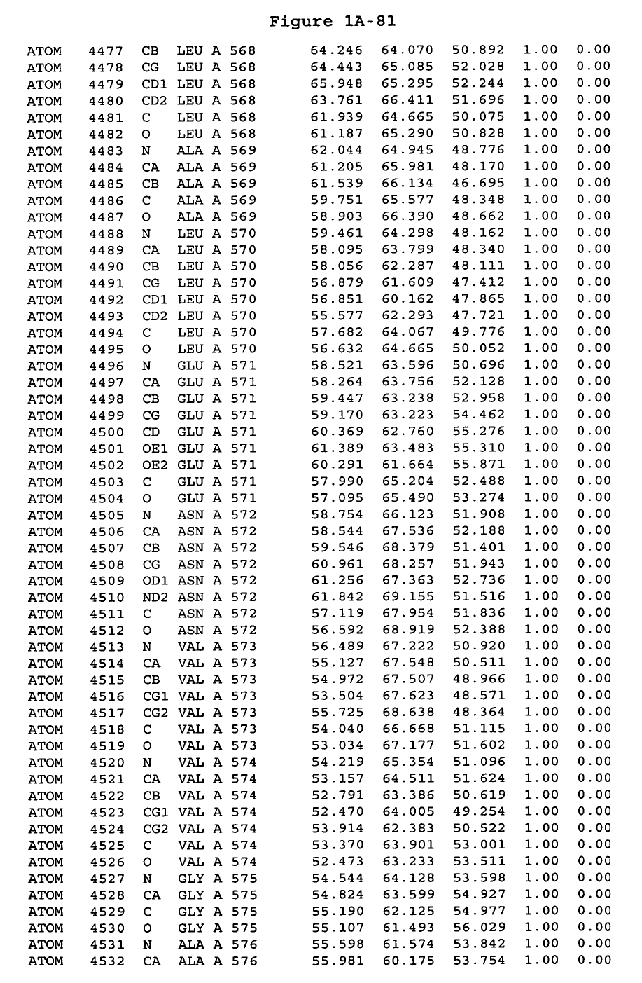

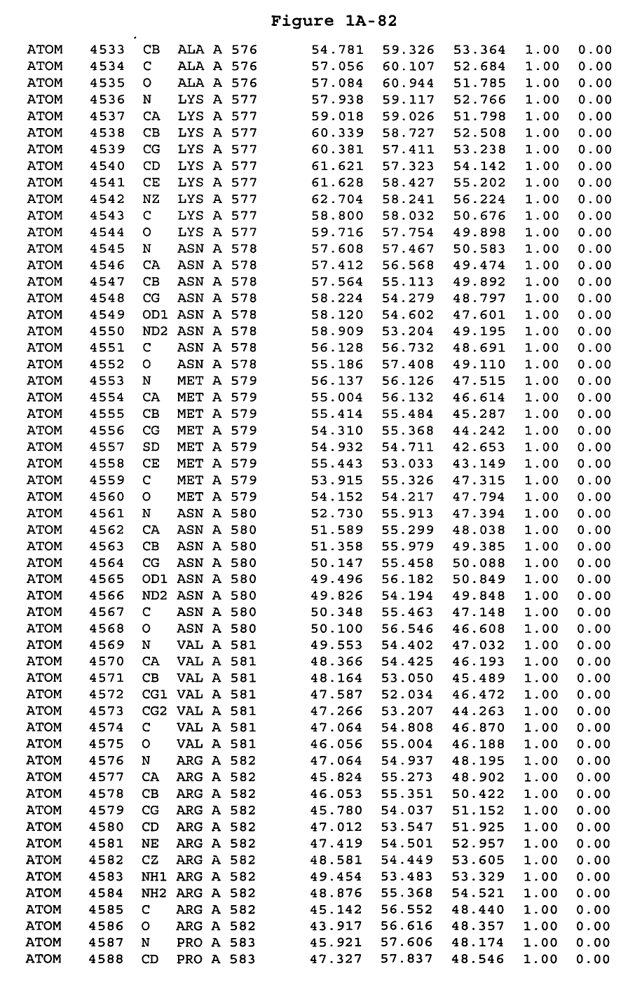

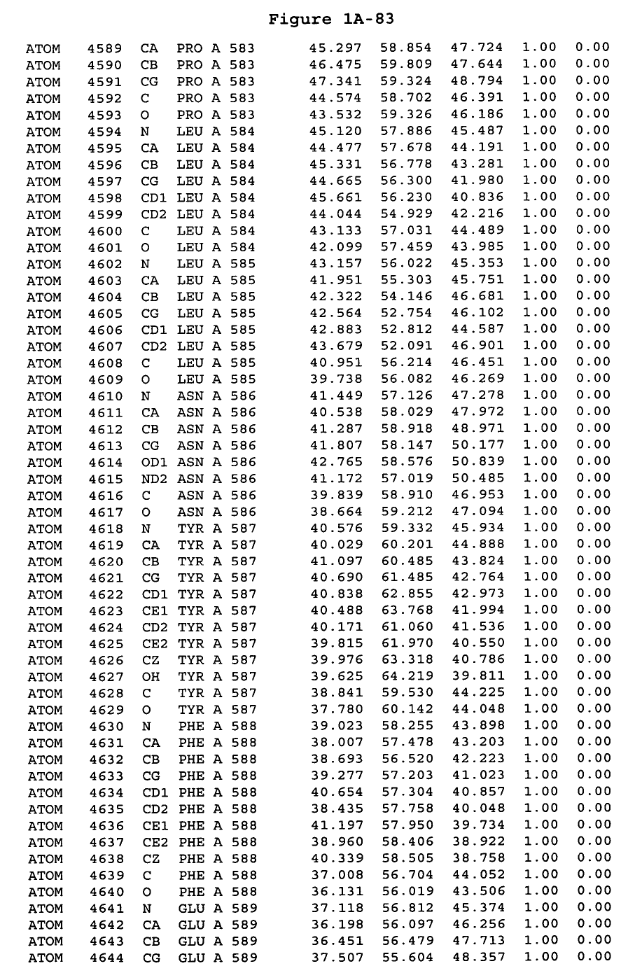

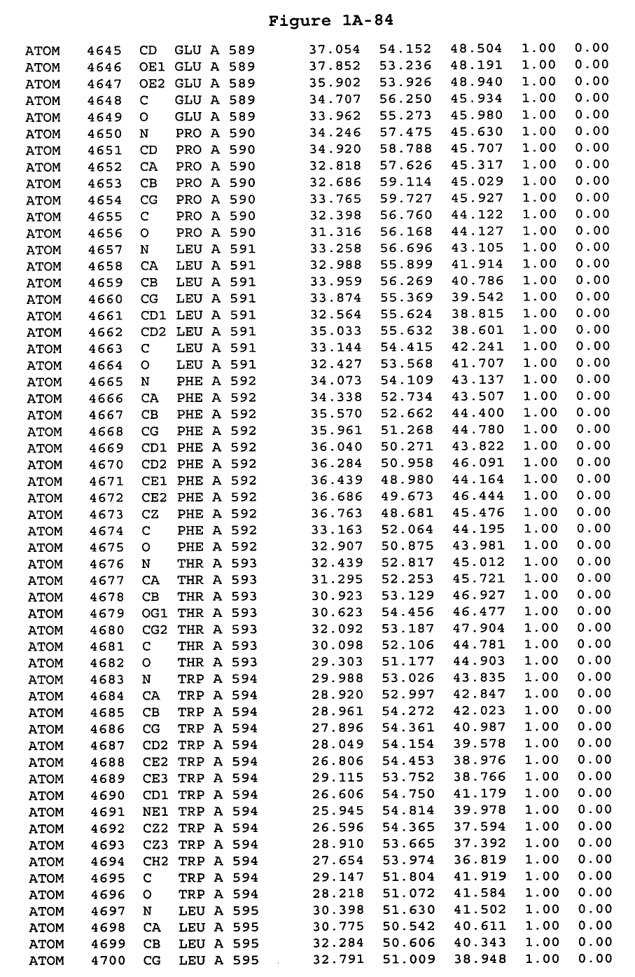

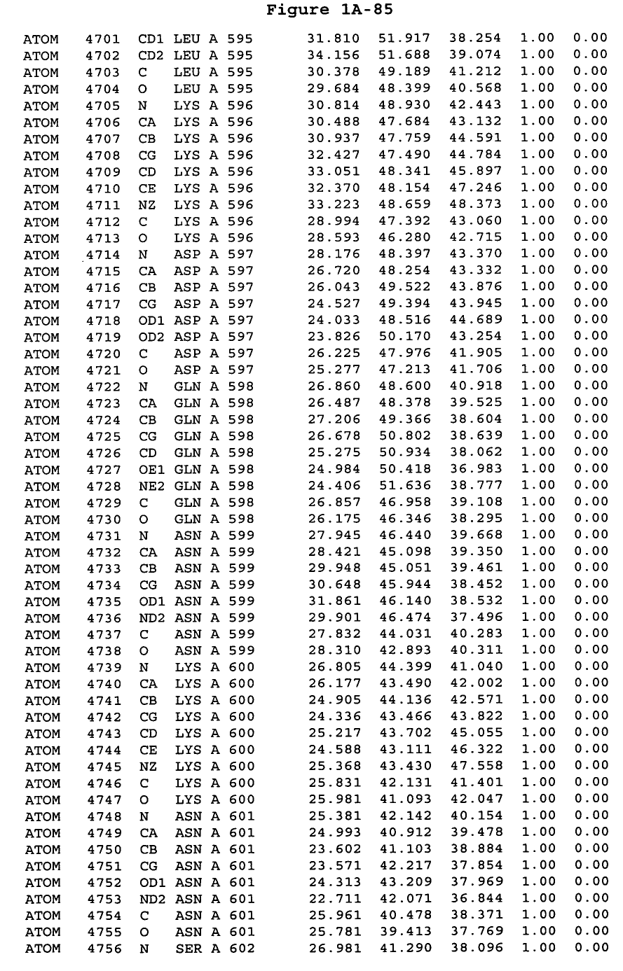

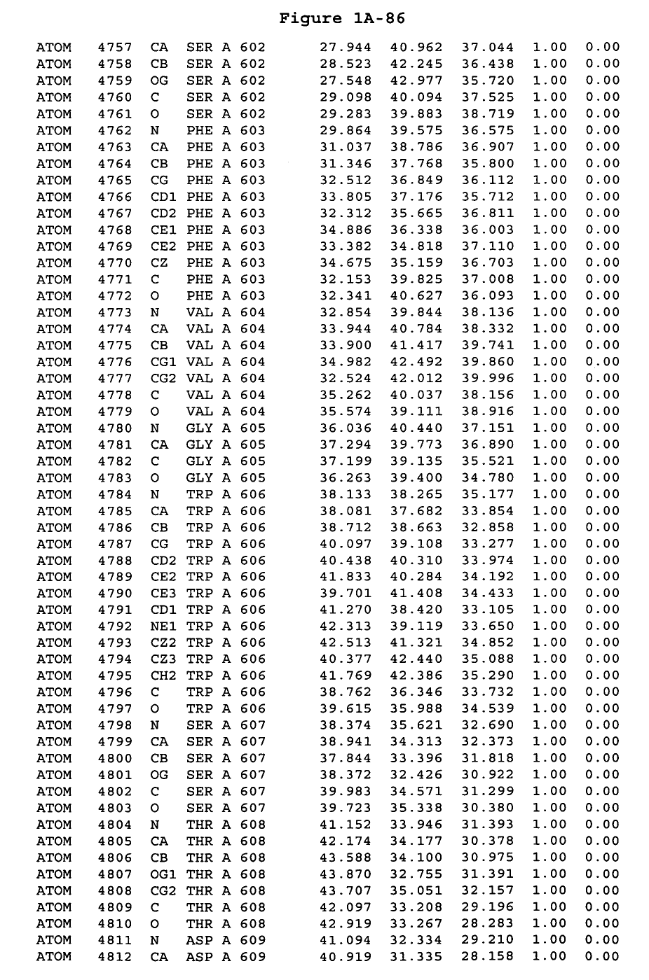

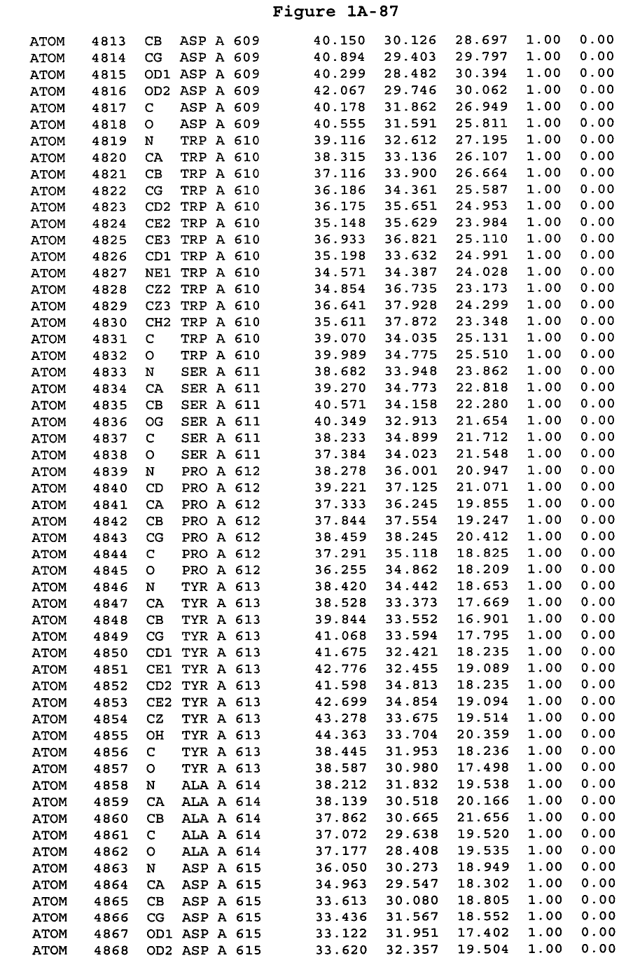

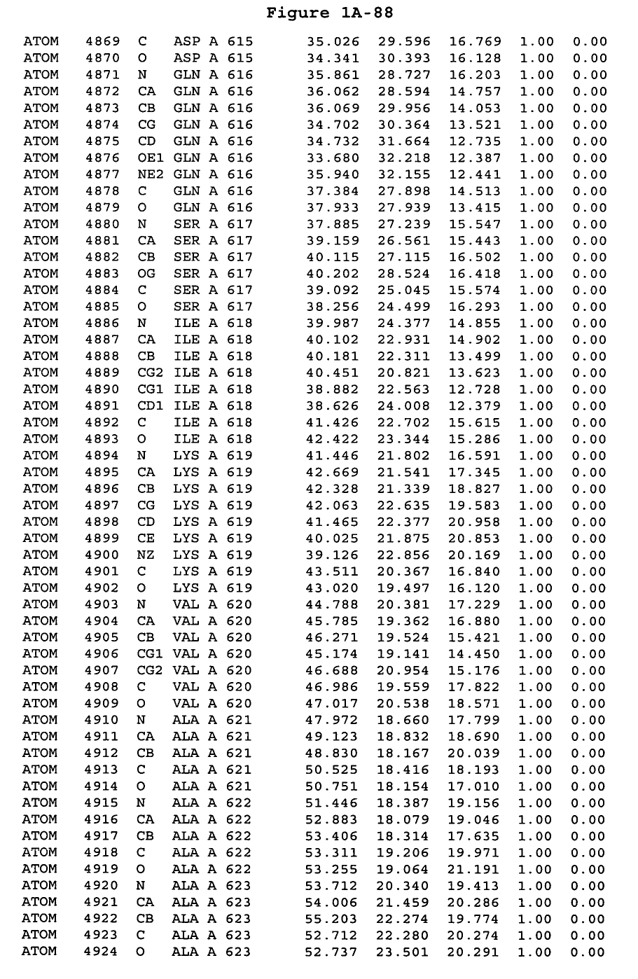

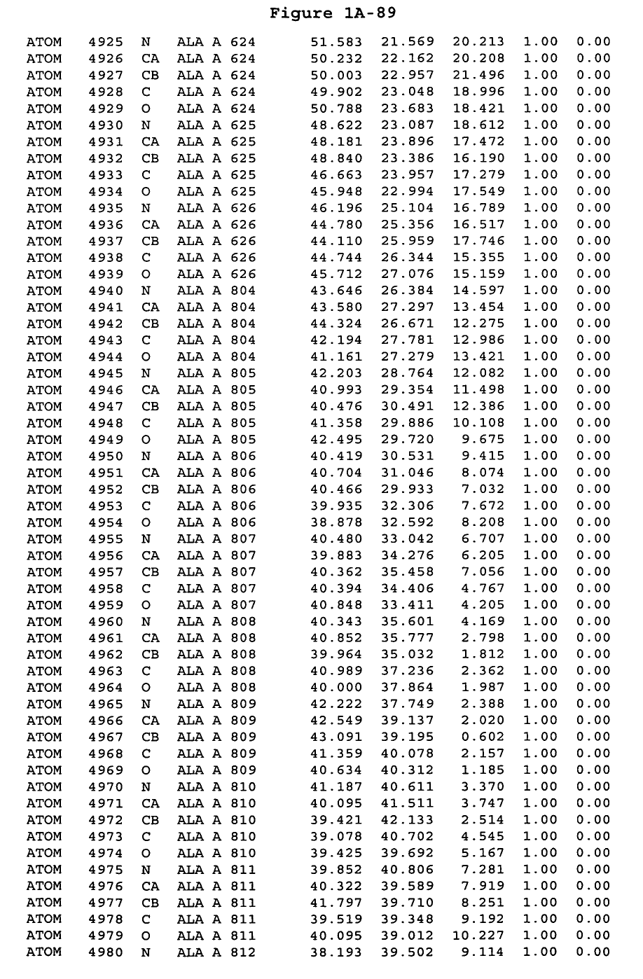

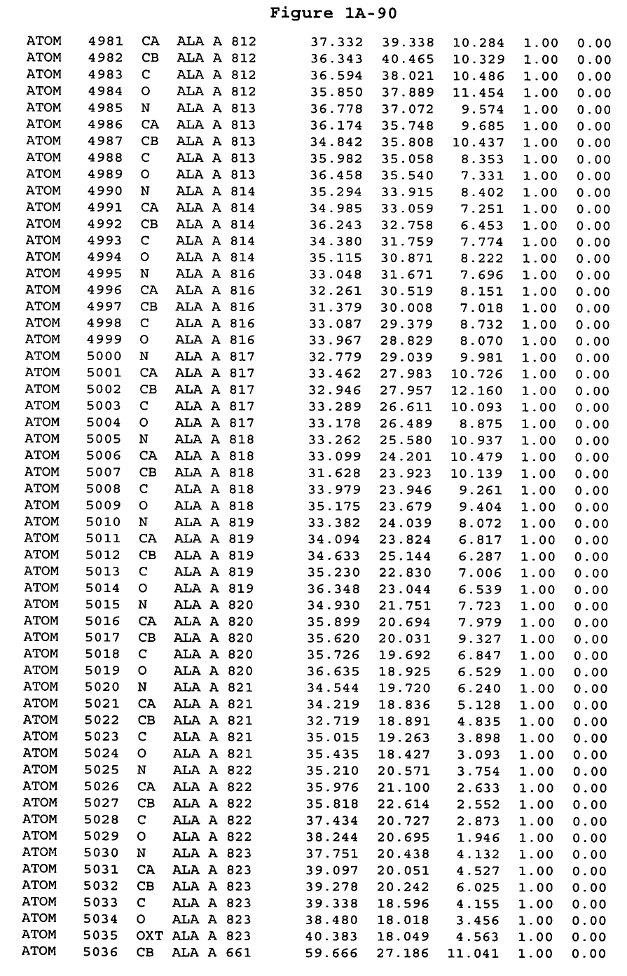

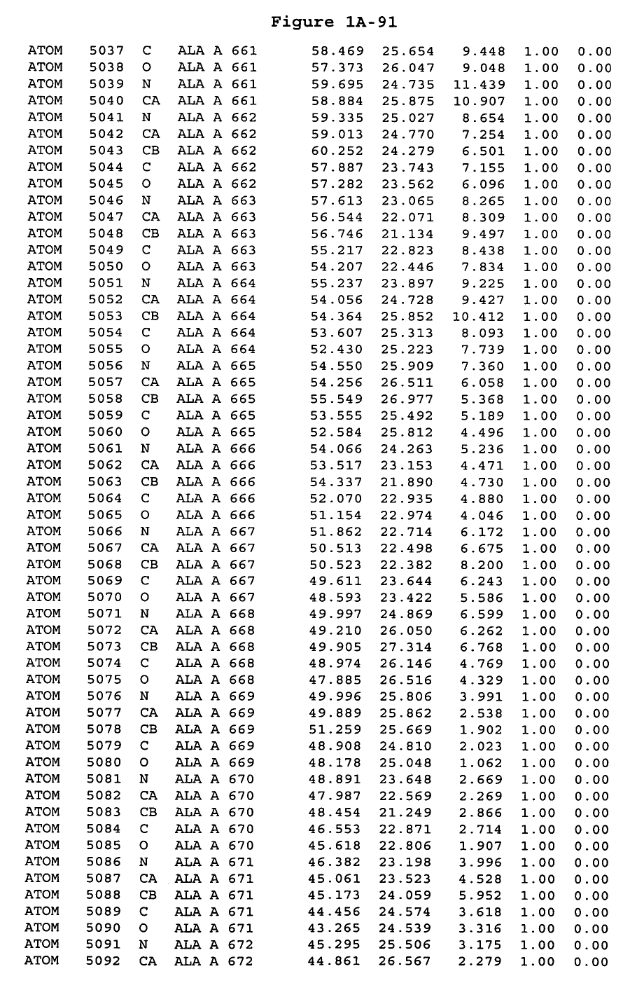

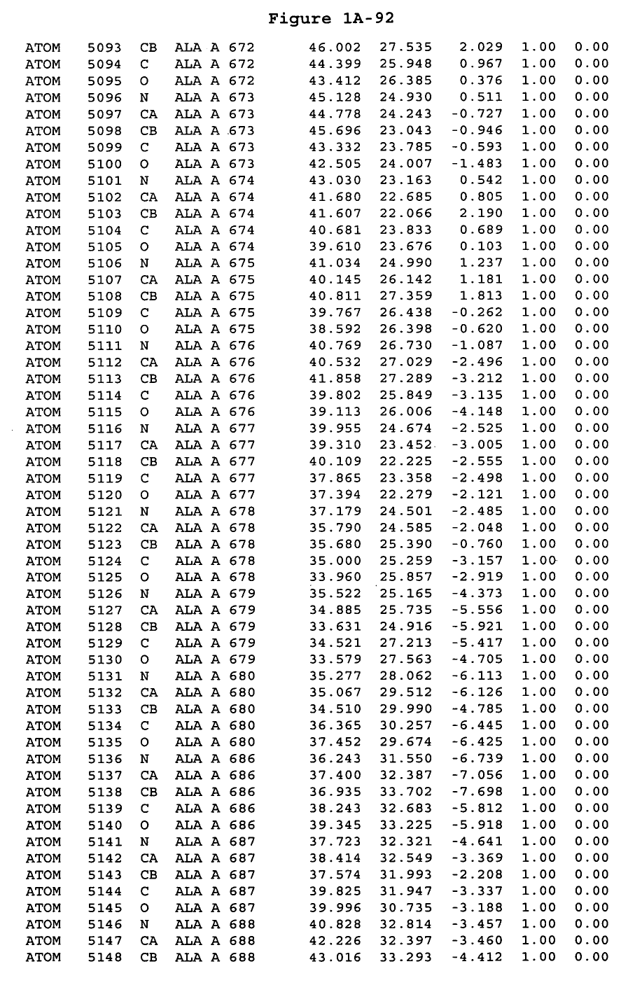

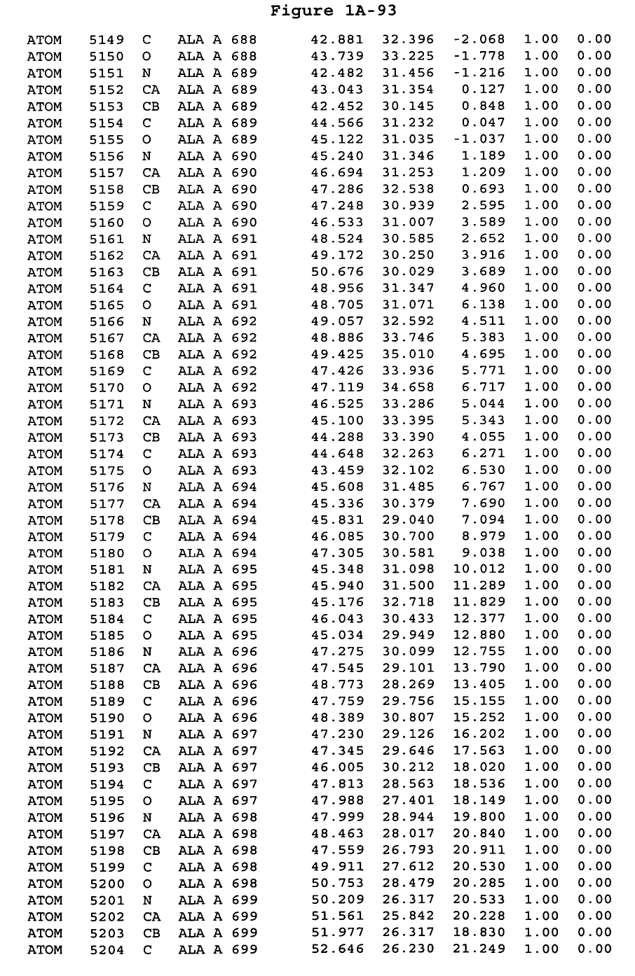

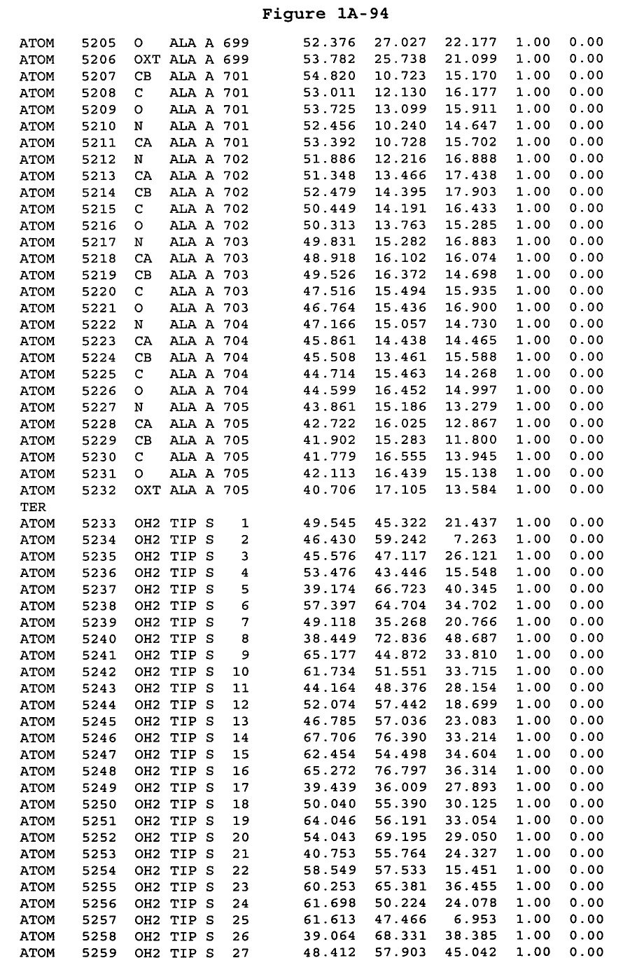

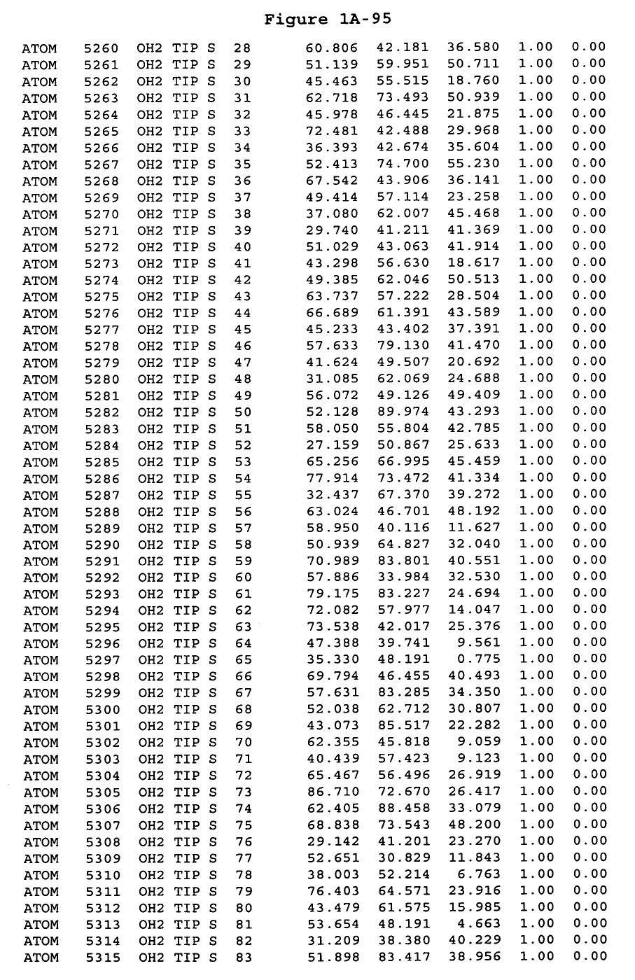

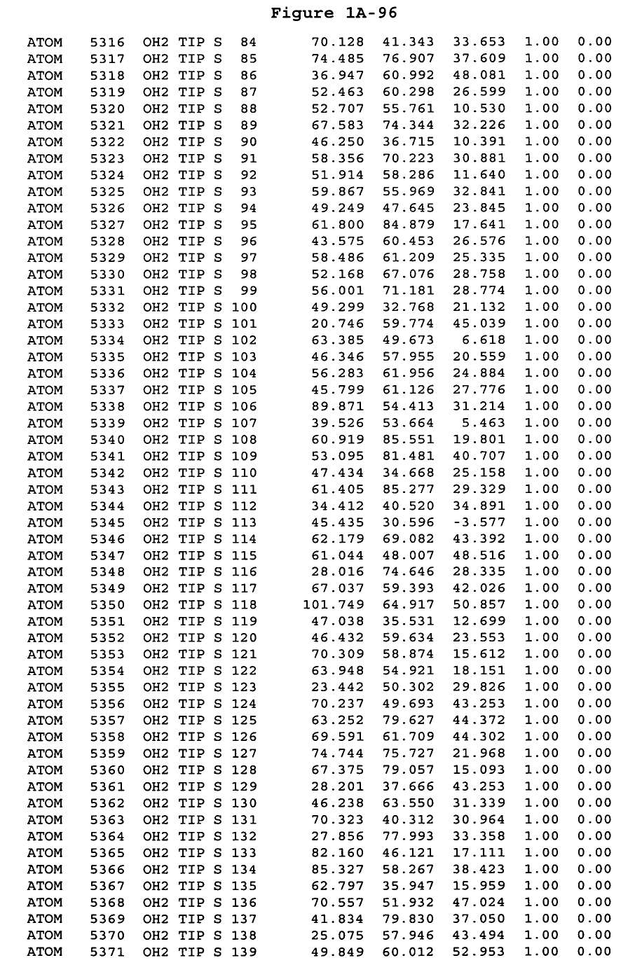

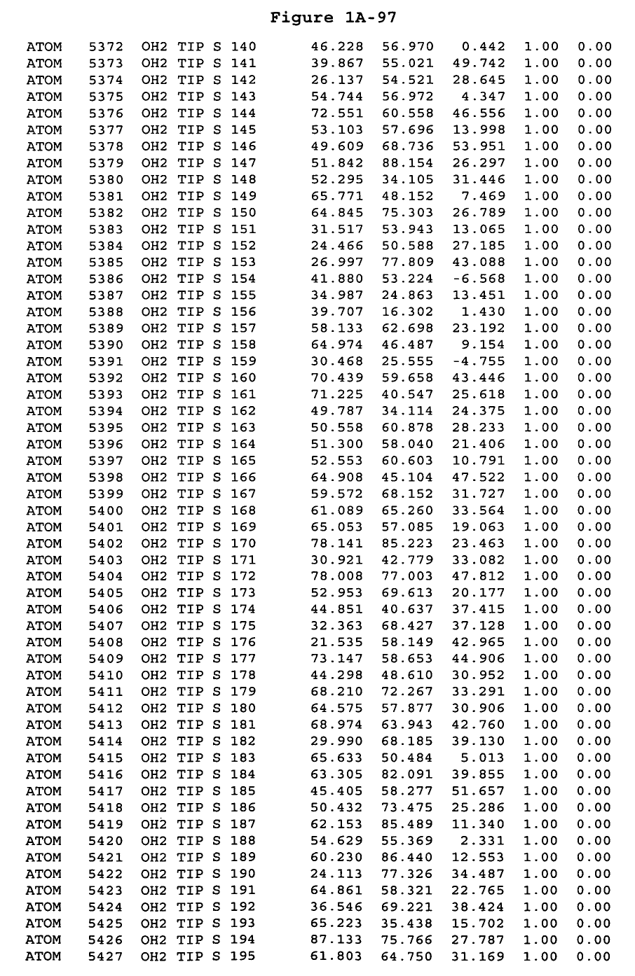

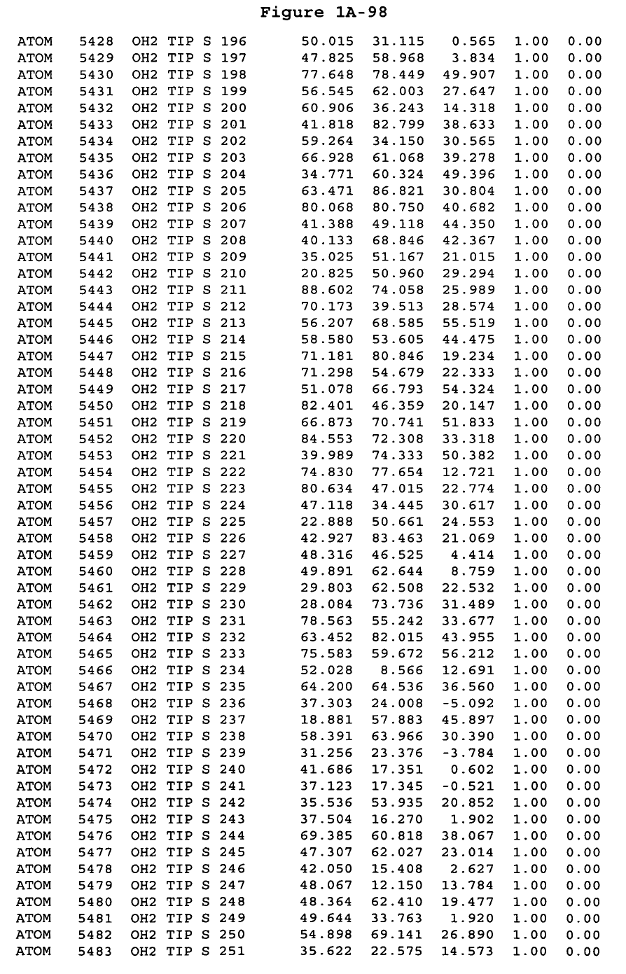

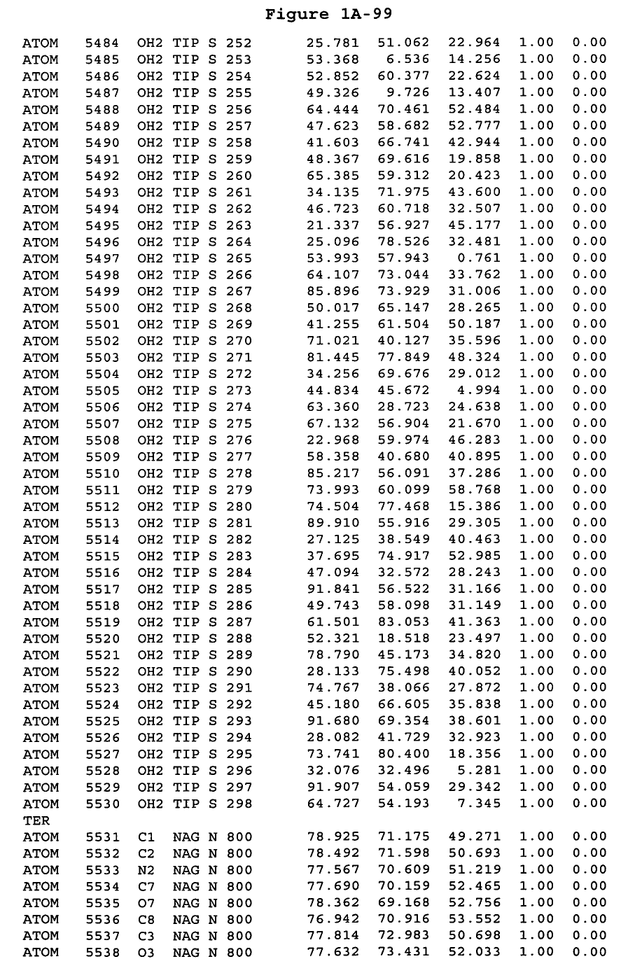

As disclosed herein, applicants have provided the three-dimensional X-ray structures of ACE2 and an ACE2-inhibitor complex. The atomic coordinate data is presented in FIGS. 1A, 2A, 3A and 3B.

To use the structure coordinates generated for the ACE2 protein or one of its binding pockets or an ACE2-like binding pocket, alone or in complex with one or more chemical entity, it may be necessary to convert the structure coordinates into a three-dimensional shape (i.e., a three-dimensional representation of these proteins, protein complexes and binding pockets). This is achieved through the use of a computer comprising commercially available software that is capable of generating three-dimensional structures of molecules or molecular complexes or portions thereof from a set of structure coordinates. These three-dimensional representations may be displayed on a computer screen.

Binding pockets, also referred to as binding sites in the present invention, are of significant utility in fields such as drug discovery. The association of natural ligands or substrates with the binding pockets of their corresponding receptors or enzymes is the basis of many biological mechanisms of action. Similarly, many drugs exert their biological effects through association with the binding pockets of receptors and enzymes. Such associations may occur with all or part of the binding pocket. An understanding of such associations will help lead to the design of drugs having more favorable associations with their target receptor or enzyme, and thus, improved biological effects. Therefore, this information is valuable in designing potential inhibitors of the binding pockets of biologically important targets. The binding pockets of this invention are important for drug design.

The conformations of ACE2 and other proteins at a particular amino acid site, along the polypeptide backbone, can be compared using well-known procedures for performing sequence alignments of the amino acids. Such sequence alignments allow for the equivalent sites on these proteins to be compared. Such methods for performing sequence alignment include, but are not limited to, the “bestfit” program and CLUSTAL W Alignment Tool, supra.

The active site binding pocket of ACE2 was originally predicted from the native human ACE2 structure coordinates (FIG. 1A). These predictions were based upon the residues found near the zinc binding site and the P1, P1′, P2, P3 binding sites (See, Examples 7 and 8). Specifically, the P1, P1′, P2 and P3 substrate binding site amino acid residues in tetra-peptide were predicted from tetra-peptide docking experiments described in Example 8.

In one embodiment, the active site binding pocket of human ACE2 comprises amino acid residues Arg 273, Phe 274, His 374, Glu 375, His 401, Glu 402, Glu 406, His 505, Tyr 510, Arg 514, Tyr 515 and Arg 518 according to FIG. 1A. In another embodiment, the active site binding pocket of human ACE2 comprises amino acid residues Arg 273, Phe 274, Glu 406, His 505, Tyr 510, Tyr 515 and Arg 518 according to FIG. 1A. In another embodiment, the active site binding pocket of human ACE2 comprises amino acid residues Arg 273, His 505 and Tyr 515 according to FIG. 1A.

In another embodiment, the active site binding pocket of human ACE2 comprises amino acid residues His 374, His 378 and Glu 402 according to FIG. 1A. These residues are in the zinc binding site.

In another embodiment, the active site binding pocket of human ACE2 comprises amino acid residues Pro 346, Thr 347, Glu 402, Phe 504, Tyr 510, Arg 514 and Tyr 515 according to FIG. 1A. These residues are in the P1 binding site. In another embodiment, the active site binding pocket comprises amino acid residues Pro 346, Thr 347 and Tyr510 according to FIG. 1A.

In another embodiment, the active site binding pocket of human ACE2 comprises amino acid residues Arg 273, Phe 274, His 345, Pro 346, Thr 371, His 374, Glu 406, Ser 409 and Arg 518 according to FIG. 1A. These residues are in the P1′ binding site. In another embodiment, the active site binding pocket of human ACE2 comprises amino acid residues Arg 273, Glu 406 and Arg 518 according to FIG. 1A.

In another embodiment, the active site binding pocket of human ACE2 comprises amino acid residues His 379, Asp 382, Tyr 385, Asn 394, His 401, Glu 402, Arg 514 according to FIG. 1A. These residues are in the P2 binding site.

In another embodiment, the active site binding pocket of human ACE2 comprises amino acid residues Phe 40, Ser 44, Thr 347, Trp 349, Asp 382, Tyr 385, Asn 394, according to FIG. 1A. These residues are in the P3 binding site. In another embodiment, the active site binding pocket of human ACE2 comprises amino acid residues Asp 382 and Asn 394 according to FIG. 1A.

In another embodiment, the active site binding pocket of human ACE2 comprises at least 3, 5, 7 or 10 amino acid residues selected from the group consisting of Phe 40, Ser 44, Trp 69, Ser 70, Leu 73, Lys 74, Ser 77, Thr 78, Leu 85, Leu 91, Thr 92, Lys 94, Leu 95, Gln 96, Gln 98, Ala 99, Leu 100, Gln 101, Gln 102, Asn 103, Gly 104, Ser 106, Asn 194, His 195, Tyr 196, Tyr 199, Tyr 202, Trp 203, Arg 204, Gly 205, Asp 206, Tyr 207, Glu 208, Val 209, Asn 210, Val 212, Arg 219, Arg 273, Phe 274, Thr 276, Tyr 279, Pro 289, Asn 290, Ile 291, Cys 344, His 345, Pro 346, Thr 347, Ala 348, Trp 349, Asp 350, Leu 351, Gly 352, Cys 361, Met 366, Asp 367, Asp 368, Leu 370, Thr 371, His 374, Glu 375, His 378, Asp 382, Tyr 385, Phe 390, Leu 391, Leu 392, Arg 393, Asn 394, Gly 395, Ala 396, Asn 397, Glu 398, Gly 399, Phe 400, His 401, Glu 402, Ala 403, Glu 406, Ser 409, Leu 410, Ala 413, Thr 414, Pro 415, Leu 418, Phe 428, Glu 430, Asp 431, Asn 432, Thr 434, Glu 435, Asn 437, Phe 438, Lys 441, Gln 442, Thr 445, Ile 446, Thr 449, Leu 450, Arg 460, Phe 504, His 505, Ser 507, Asn 508, Asp 509, Tyr 510, Ser 511, Arg 514, Tyr 515, Arg 518, Thr 519, Gln 522, His 540, Lys 541, Lys 562, Ser 563, Glu 564, Pro 565, Trp 566 and Tyr 587 according to FIG. 1A.

After the ACE2-inhibitor1 complex structure was refined, it was also possible to predict the binding pocket from the structure coordinates of this complex (FIG. 3A or 3B).

In another embodiment, the binding pocket comprises amino acids N149, D269, R273, H345, P346, A348, D367, H374, E375, H378, E402, F504, H505, Y510 and Y515 according to the structure of ACE2-inhibitor1 complex in FIG. 3A or 3B. The above-identified amino acid residues were within 5 Å (“5 Å sphere amino acids”) of the inhibitor bound in the binding pockets. These residues were identified using the program QUANTA (Molecular Simulations, Inc., San Diego, Calif. ©1998, 2000; Accelrys ©2001, 2002), 0 (T. A. Jones et al., Acta Cryst., A47, pp. 110-119 (1991)) and RIBBONS (Carson, J. Appl. Cryst., 24, pp. 958-961 (1991)), which allow the display and output of all residues within 5 Å from the inhibitor.

In another embodiment, the binding pocket comprises amino acids L144, E145, N149, M152, A153, D269, M270, W271, R273, F274, N277, H345, P346, T347, A348, K363, T365, D367, D368, T371, H374, E375, H378, E402, F504, H505, Y510, F512, R514, Y515 and R518 according to the structure of ACE2-inhibitor1 complex in FIG. 3A or 3B. These amino acids residues were within 8 Å (“8 Å sphere amino acids”) of the inhibitor bound in the ATP-binding pockets. These residues were identified using the programs QUANTA, O and RIBBONS, supra.

The binding pocket comprises the amino acid residues that are unique (non-conserved between homologues) to a molecule; these residues allow that binding pocket to adopt a unique shape and allow for distinct binding site specificity. The binding pocket may comprise the amino acid residues found within the near vicinity (5 Å or 8 Å) of a bound inhibitor. The binding pocket may also comprise residues which are shown by the structure coordinates to be important for maintaining the structural integrity of the amino acid residues that either directly bind to inhibitor or form the binding pocket. Therefore, in one embodiment, the binding pocket of human ACE2 comprises amino acids residues N149, D269, R273, F274, H345, P346, A348, D367, T371, H374, E375, H378, E398, E402, R481, L503, F504, H505, Y510, S511, F512, Y515 and E564 according to FIG. 3A or 3B. The importance of these additional residues is noted in Example 9. Residue F274 and T371 are not conserved in tACE and are positioned to line the S1′ site of the ACE2-inhibitor1 structure; therefore, these residues may be responsible for binding site specificity. Residue E398 and S511 form a hydrogen bond and project into the location where a second chloride anion binding site is located in the tACE-inhibitor structure; therefore, in part distinguishing tACE-inhibitor binding from ACE2-inhibitor binding. Residue E564 is the only non-conserved residue of the residues that act as mechnical hinges upon active site closure (other hinge residues include A396, N397, L539, H540, P565 and W566). Residue K481 in tACE is a lysine. Residue L503 and F512, as compared with K511 and Y520 (the corresponding residues in tACE), lack the ability to form hydrogen bonds with the terminal carboxylate of the inhibitor. Without being bound by theory, this may contribute to binding site specificity in ACE2. In another embodiment, the binding pocket of human ACE2 comprises amino acids residues N149, D269, R273, F274, H345, P346, A348, D367, T371, H374, E375, H378, E398, E402, R481, L503, F504, H505, Y510, S511, F512 and Y515 according to FIG. 3A or 3B.

In another embodiment, the binding pocket of human ACE2 comprises amino acids residues N149, D269, R273, F274, H345, P346, A348, D367, T371, H374, E375, H378, E402, F504, H505, Y510, F512, and Y515 according to FIG. 3A or 3B. In a preferred embodiment, the binding pocket of human ACE2 comprises amino acids residues N149, D269, R273, F274, P346, T371, Y510, and F512 according to FIG. 3A or 3B.

In one embodiment, the binding pocket of human ACE2 additionally comprises amino acid residues that are shown in FIG. 10. Accordingly, in one embodiment, the binding pocket of human ACE2 comprises amino acid residues N149, D269, R273, F274, H345, P346, A348, D367, T371, H374, E375, H378, E398, E402, R481, L503, F504, H505, Y510, S511, F512, R514, Y515 and E564. In one embodiment, the binding pocket of human ACE2 comprises amino acid residues N149, D269, R273, F274, P346, T371, E398, R481, L503, Y510, S511, F512, and E564.

In another embodiment, the binding pocket of human ACE2 comprises amino acid residues N149, D269, R273, F274, H345, P346, A348, D367, T371, H374, E375, H378, E402, F504, H505, Y510, F512, R514, and Y515. In one embodiment, the binding pocket of human ACE2 comprises amino acid residues R273, F274, H345, P346, D367, T371, H374, E375, H378, E402, H505, Y510, R514 and Y515.

In one embodiment, the binding pocket of human ACE2 comprises amino acid residues R273, F274, H345, P346, T371, H374, E375, H378, E402, H505, and Y515. In another embodiment, the binding pocket of human ACE2 comprises amino acid residues R273, F274, H345, P346, T371, H374, E375, H378, E402, H505, Y510 and Y515. In one embodiment, the binding pocket of human ACE2 comprises amino acid residues R273, F274, P346, and T371.

It will be readily apparent to those of skill in the art that the numbering of amino acid residues in other homologues of human ACE2 may be different than that set forth for human ACE2. Corresponding amino acid residues in homologues of ACE2 are easily identified by visual inspection of the amino acid sequences or by using commercially available homology software programs. Homologues of ACE2 include, for example, ACE2 from other species, such as non-humans primates, mouse, rat, etc.

Those of skill in the art understand that a set of structure coordinates for an enzyme or an enzyme-complex or a portion thereof, is a relative set of points that define a shape in three dimensions. Thus, it is possible that an entirely different set of coordinates could define a similar or identical shape. Moreover, slight variations in the individual coordinates will have little effect on overall shape. In terms of binding pockets, these variations would not be expected to significantly alter the nature of ligands that could associate with those pockets.

The variations in coordinates discussed above may be generated because of mathematical manipulations of the ACE2 structure coordinates. For example, the structure coordinates set forth in FIGS. 1A, 2A, 3A or 3B could undergo crystallographic permutations, fractionalization, integer additions or subtractions, inversion, or any combination of the above.

Alternatively, modifications in the crystal structure due to mutations, additions, substitutions, and/or deletions of amino acids, or other changes in any of the components that make up the crystal could also account for variations in structure coordinates. If such variations are within a certain root mean square deviation as compared to the original coordinates, the resulting three-dimensional shape is considered encompassed by this invention. Thus, for example, a ligand that bound to the binding pocket of ACE2 would also be expected to bind to another binding pocket whose structure coordinates defined a shape that fell within the acceptable root mean square deviation.

Various computational analyses may be necessary to determine whether a molecule or the binding pocket or portion thereof is sufficiently similar to the ACE2 binding pockets described above. Such analyses may be carried out using well known software applications, such as ProFit (A. C. R. Martin, SciTech Software, ProFit version 1.8, University College London, http://www.bioinf.org.uk/software), Swiss-Pdb Viewer (Guex et al., Electrophoresis, 18, pp. 2714-2723 (1997)), the Molecular Similarity application of QUANTA (Molecular Simulations, Inc., San Diego, Calif. ©1998, 2000; Accelrys ©2001, 2002) and as described in the accompanying User's Guide, which are incorporated herein by reference.

The above programs permit comparisons between different structures, different conformations of the same structure, and different parts of the same structure. The procedure used in QUANTA (Molecular Simulations, Inc., San Diego, Calif. ©1998, 2000; Accelrys ©2001, 2002) and Swiss-Pdb Viewer to compare structures is divided into four steps: 1) load the structures to be compared; 2) define the atom equivalences in these structures; 3) perform a fitting operation on the structures; and 4) analyze the results.

The procedure used in ProFit to compare structures includes the following steps: 1) load the structures to be compared; 2) specify selected residues of interest; 3) define the atom equivalences in the selected residues; 4) perform a fitting operation on the selected residues; and 5) analyze the results.

Each structure in the comparison is identified by a name. One structure is identified as the target (i.e., the fixed structure); all remaining structures are working structures (i.e., moving structures). Since atom equivalency within QUANTA (Molecular Simulations, Inc., San Diego, Calif. ©1998, 2000; Accelrys ©2001, 2002) is defined by user input, for the purpose of this invention we will define equivalent atoms as protein backbone atoms N, C, O and Ca for all corresponding amino acids between the two structures being compared.

The corresponding amino acids may be identified by sequence alignment programs such as the “bestfit” program available from the Genetics Computer Group which uses the local homology algorithm described by Smith and Waterman in Advances in Applied Mathematics 2, 482 (1981), which is incorporated herein by reference. A suitable amino acid sequence alignment will require that the proteins being aligned share minimum percentage of identical amino acids. Generally, a first protein being aligned with a second protein should share in excess of about 35% identical amino acids (Hanks et al., Science, 241, 42 (1988); Hanks and Quinn, Methods in Enzymology, 200, 38 (1991)). The identification of equivalent residues can also be assisted by secondary structure alignment, for example, aligning the α-helices, β-sheets in the structure. The program Swiss-Pdb Viewer has its own best fit algorithm that is based on secondary sequence alignment.

When a rigid fitting method is used, the working structure is translated and rotated to obtain an optimum fit with the target structure. The fitting operation uses an algorithm that computes the optimum translation and rotation to be applied to the moving structure, such that the root mean square difference of the fit over the specified pairs of equivalent atom is an absolute minimum. This number, given in angstroms, is reported by the above programs. The Swiss-Pdb Viewer program sets an RMSD cutoff for eliminating pairs of equivalent atoms that have high RMSD values. An RMSD cutoff value can be used to exclude pairs of equivalent atoms with extreme individual RMSD values. In the program ProFit, the RMSD cutoff value can be specified by the user.

For the purpose of this invention, any molecule, molecular complex, binding pocket, motif, domain thereof or portion thereof that is within a root mean square deviation for backbone atoms (N, Ca, C, O) when superimposed on the relevant backbone atoms described by structure coordinates listed in FIGS. 1A, 2A, 3A or 3B are encompassed by this invention.

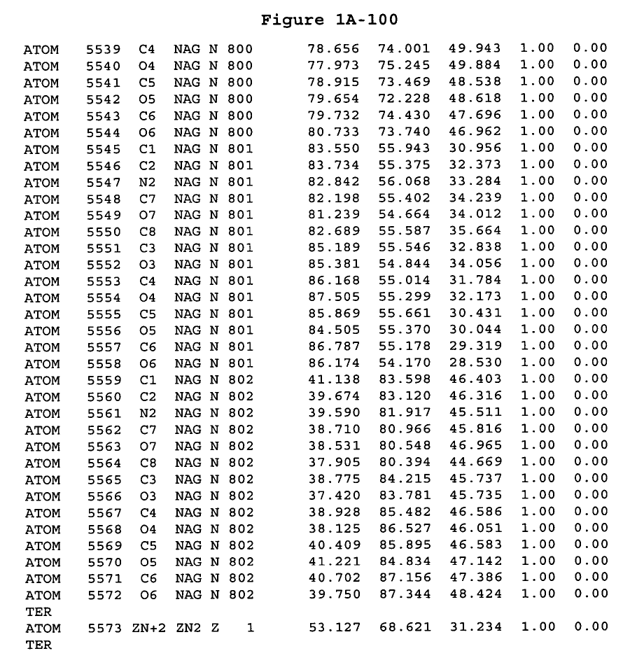

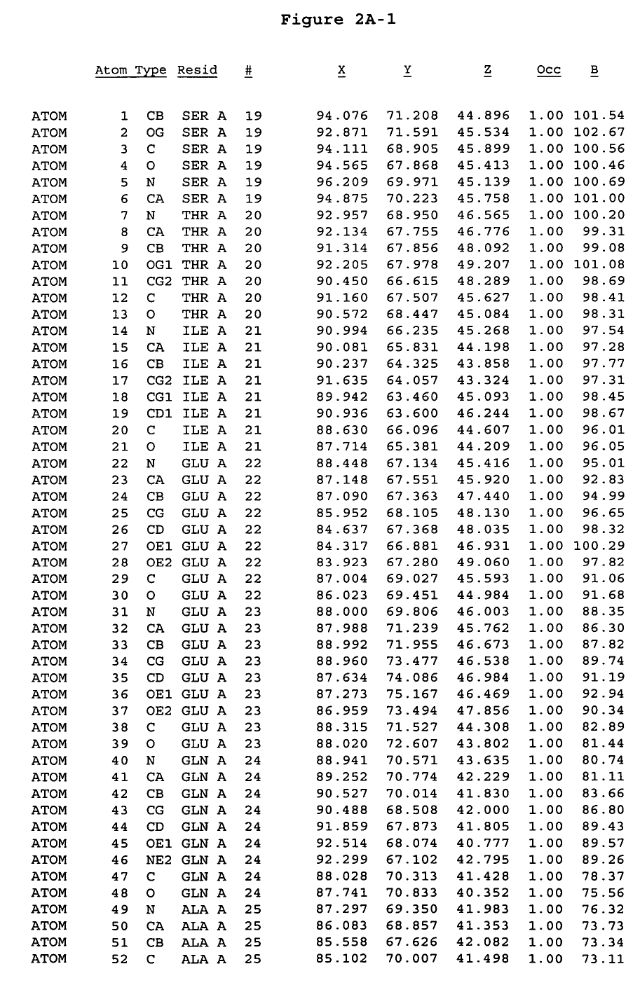

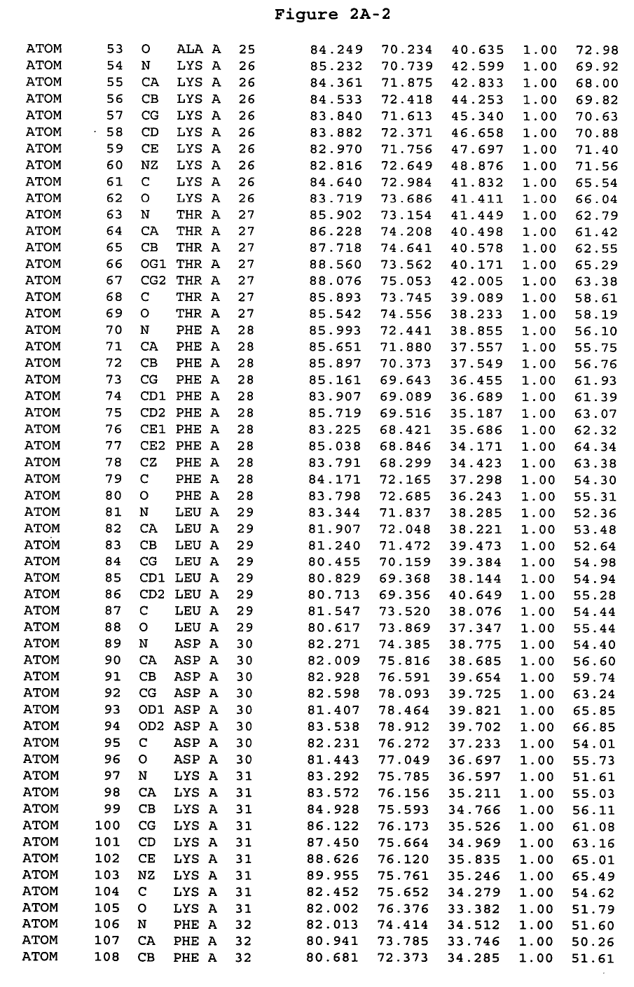

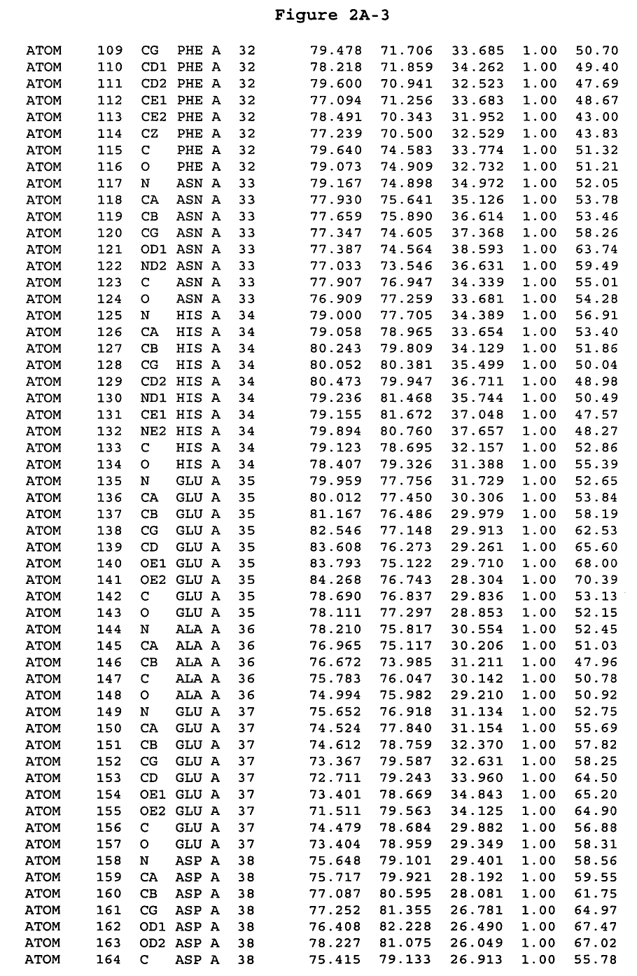

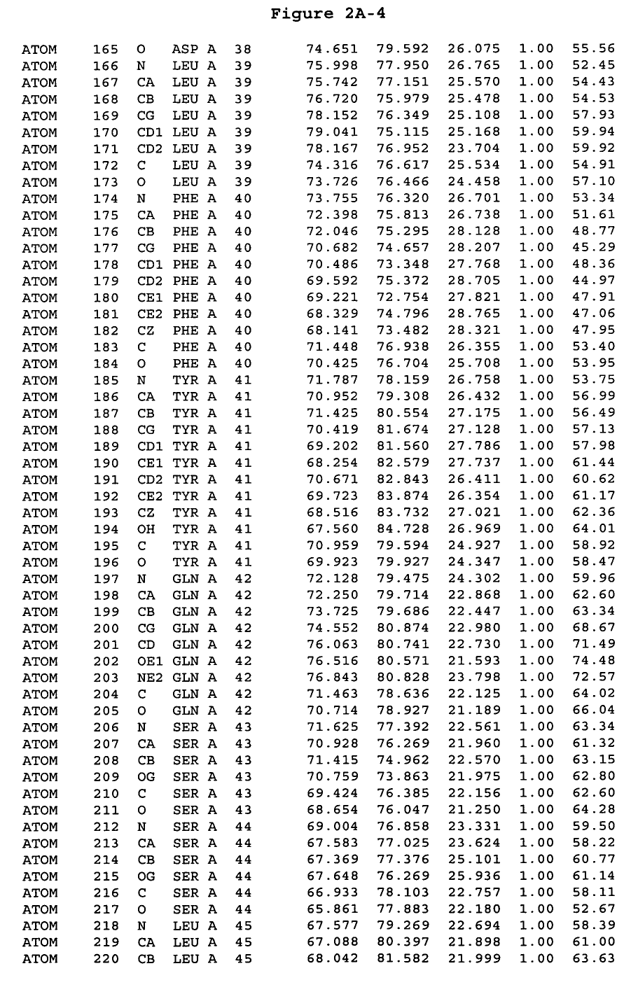

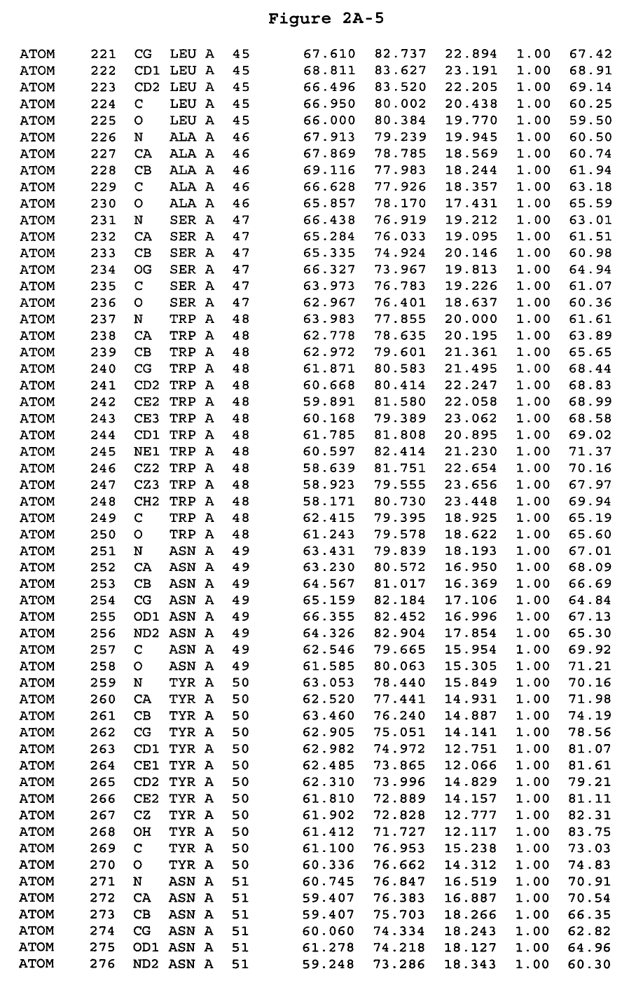

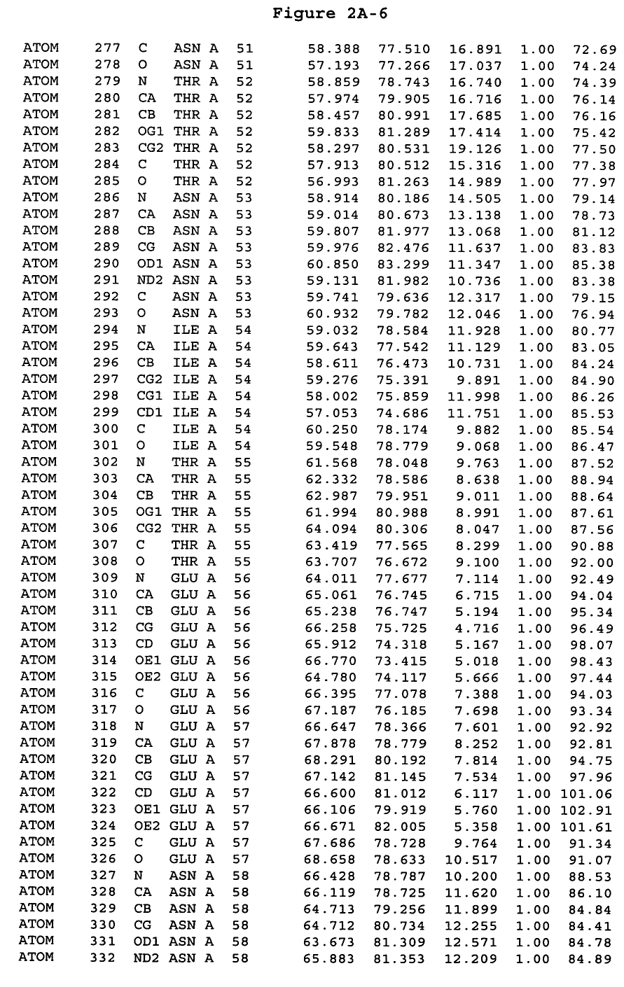

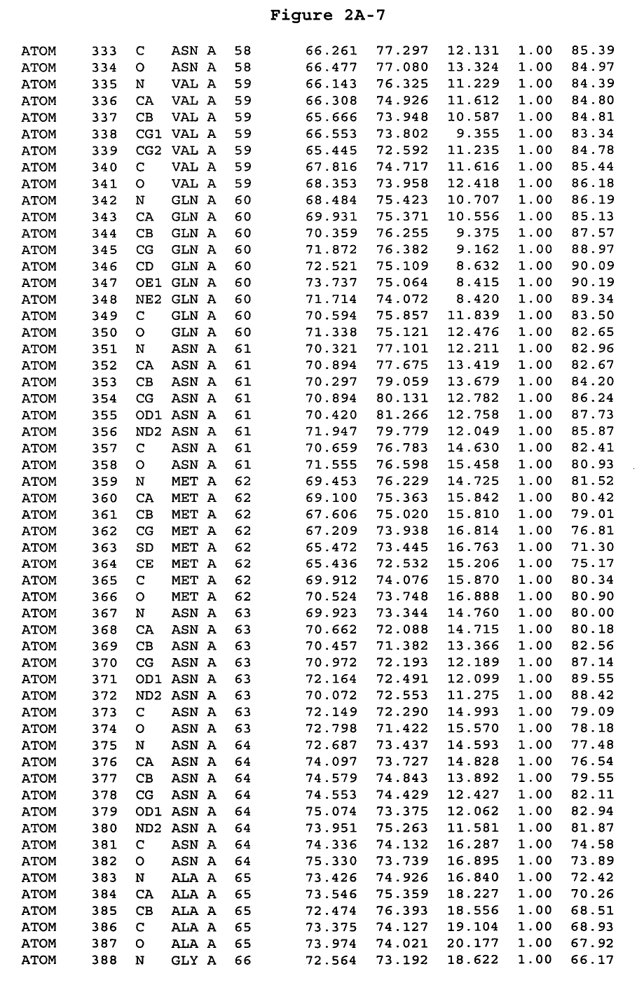

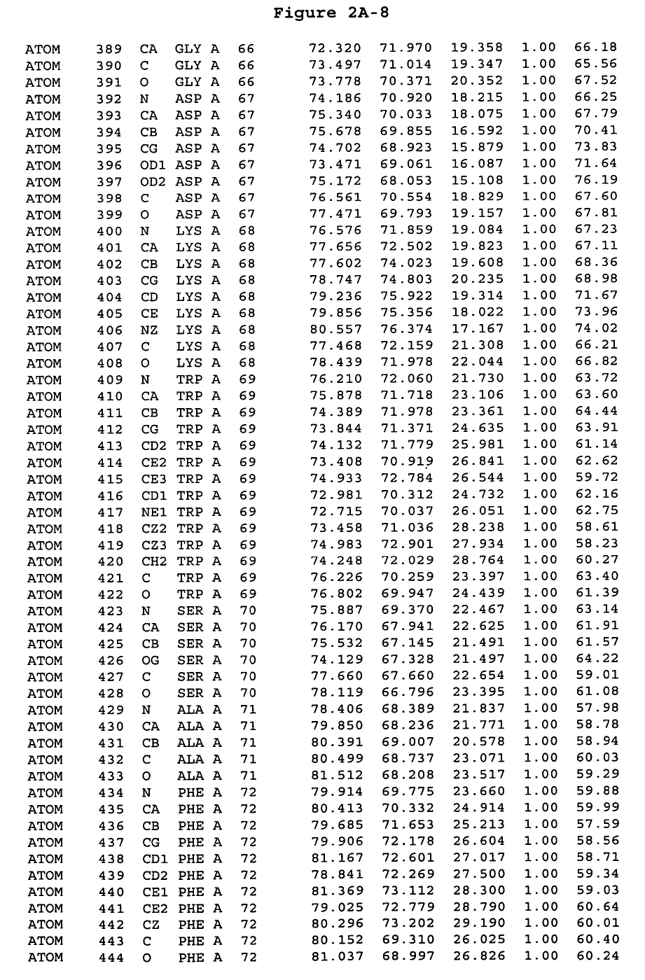

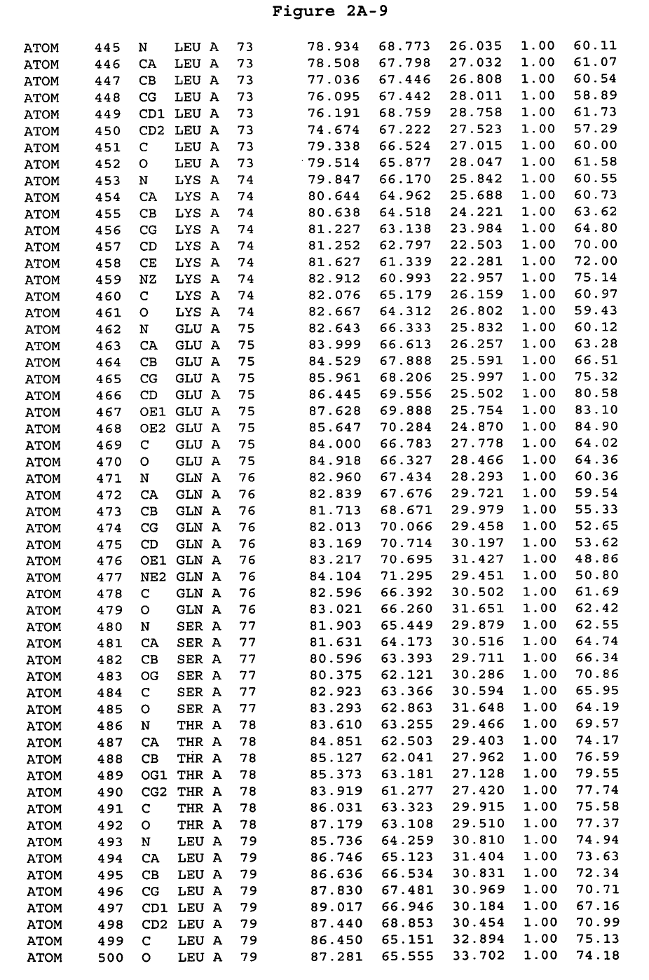

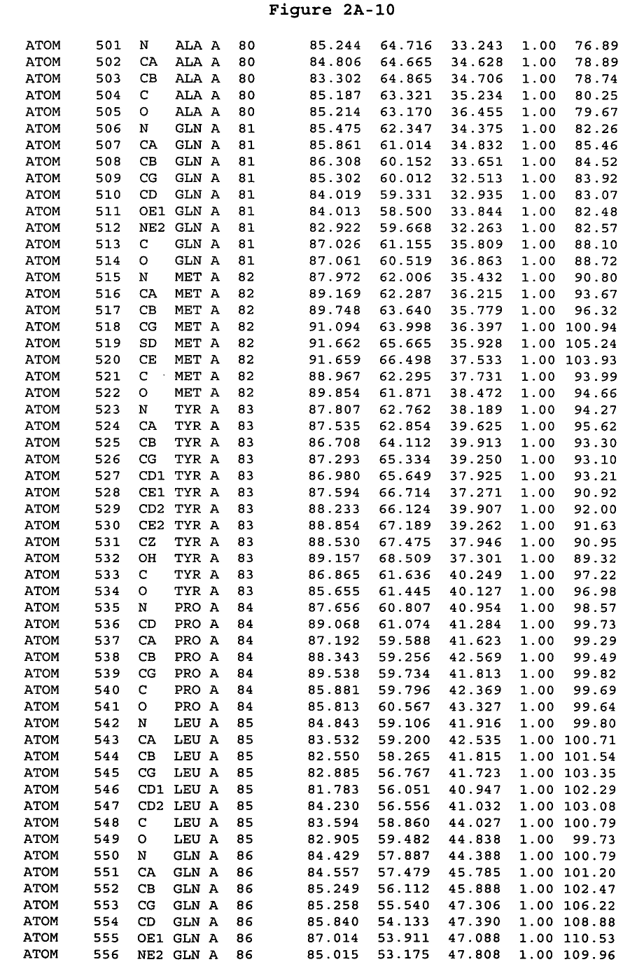

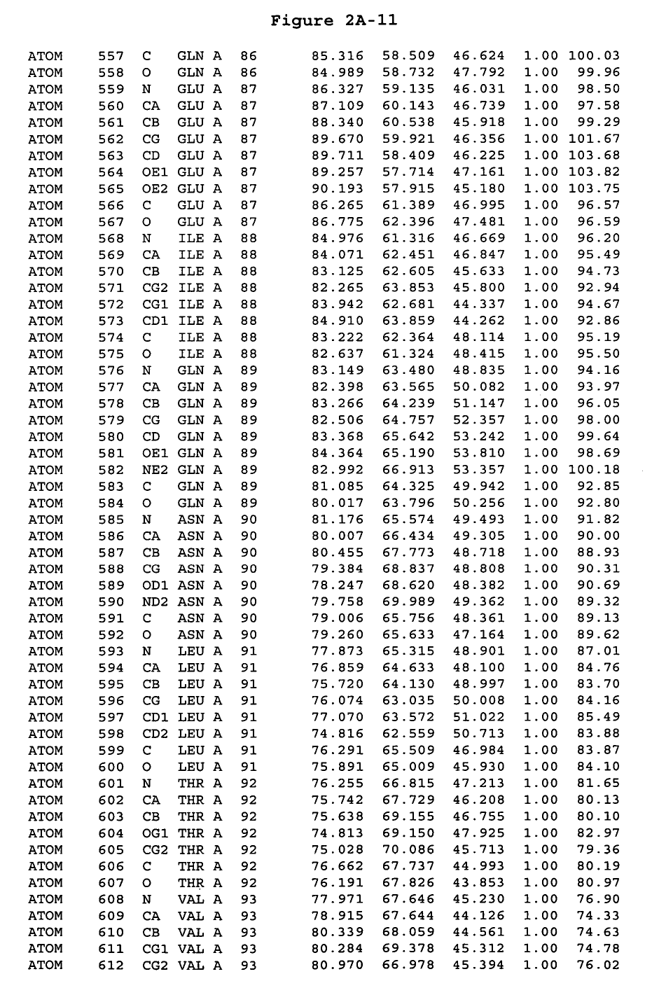

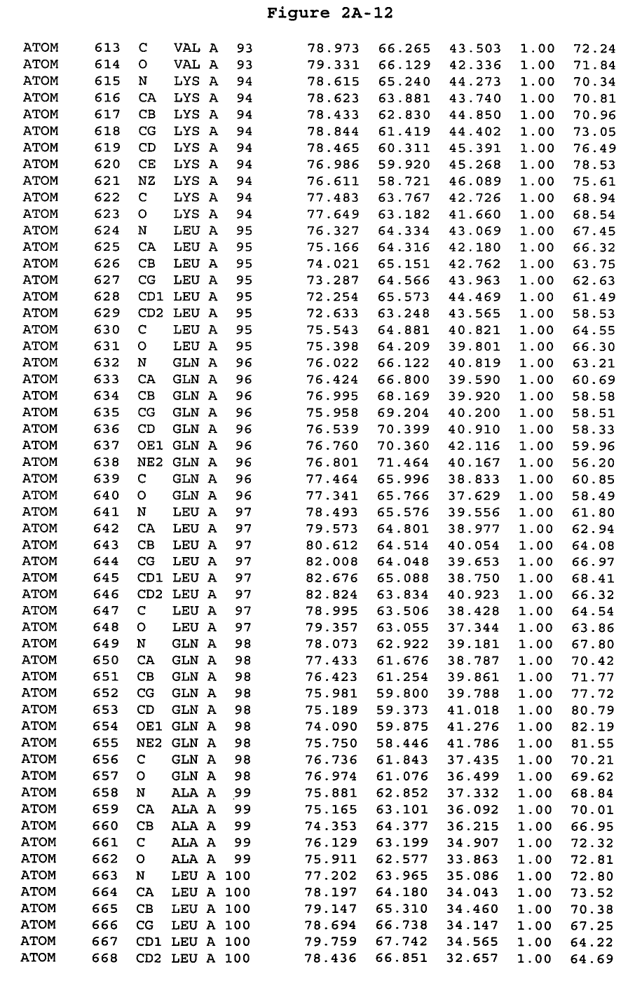

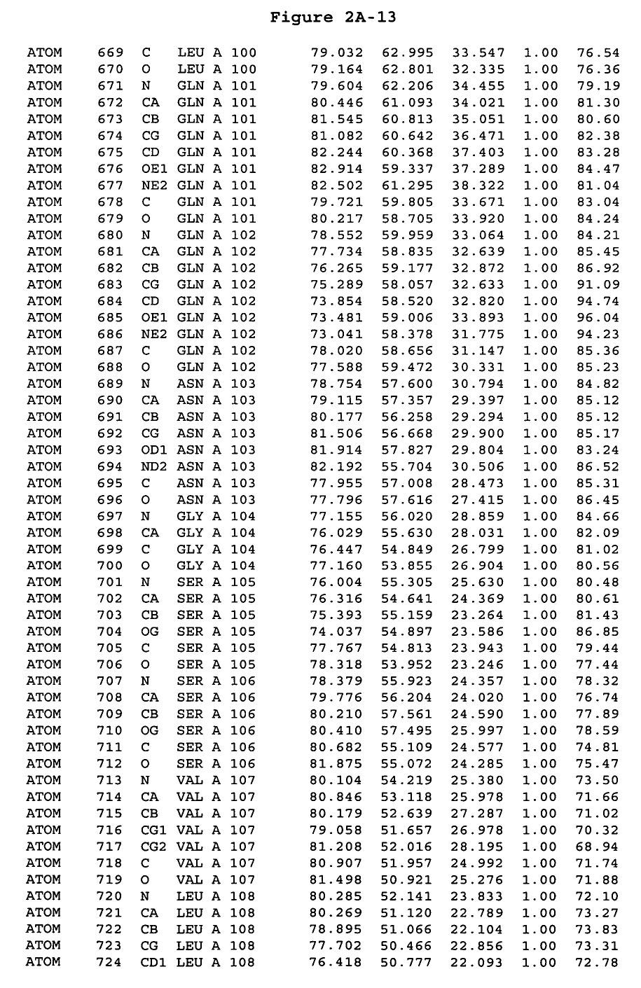

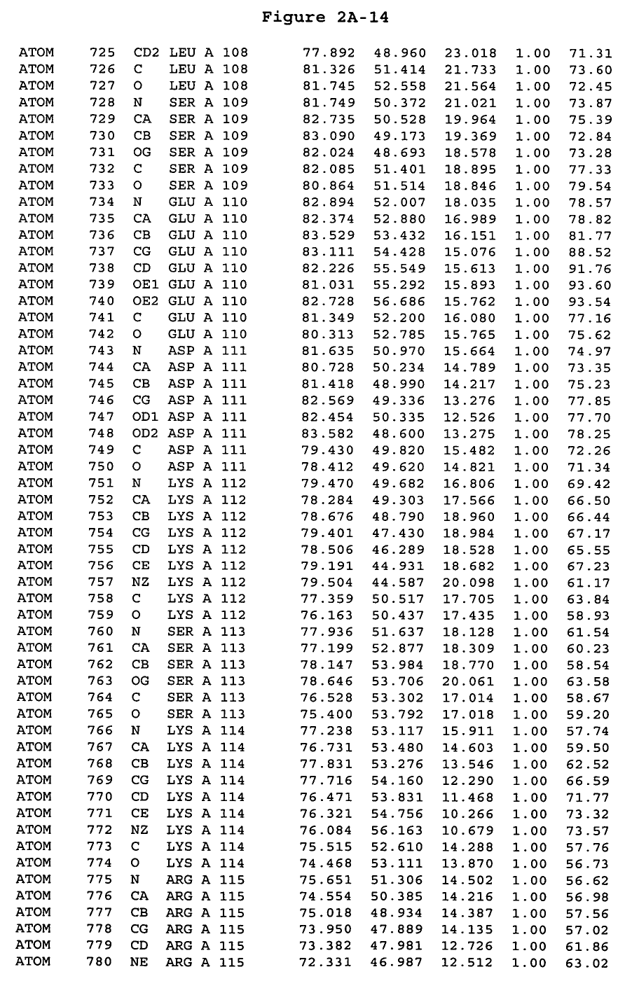

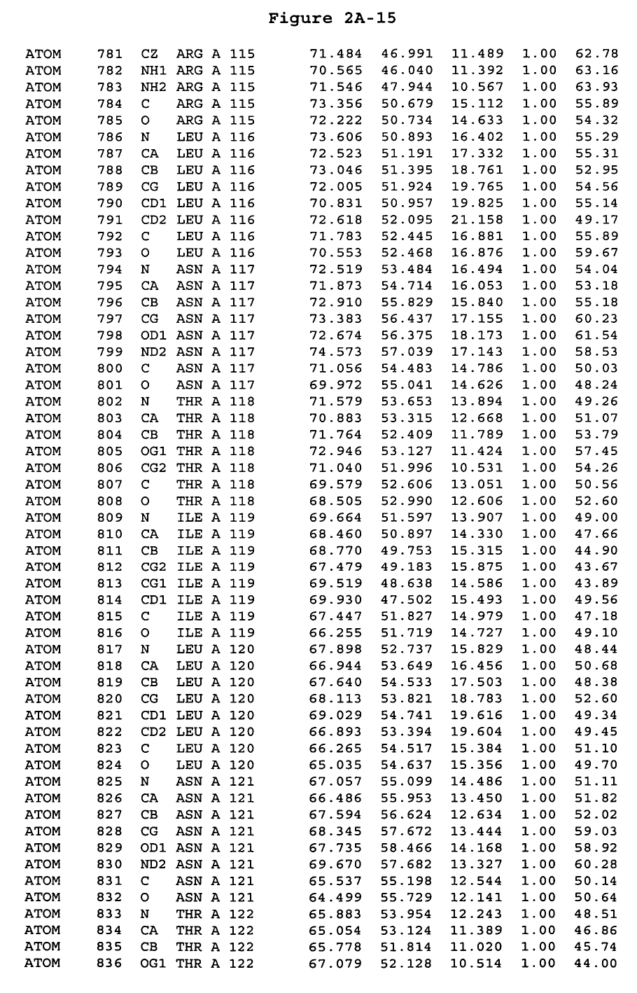

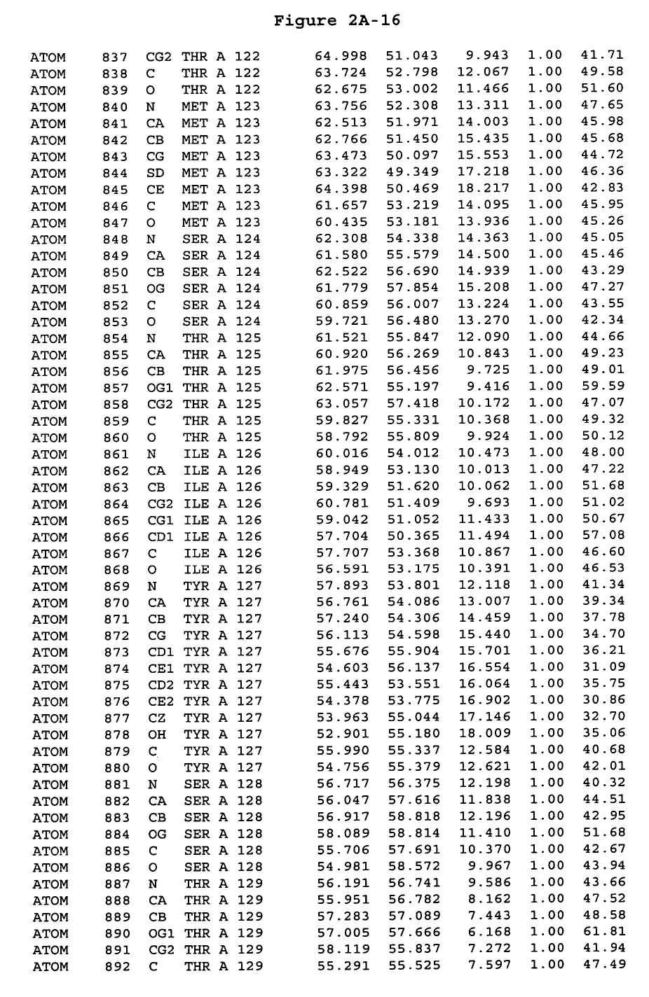

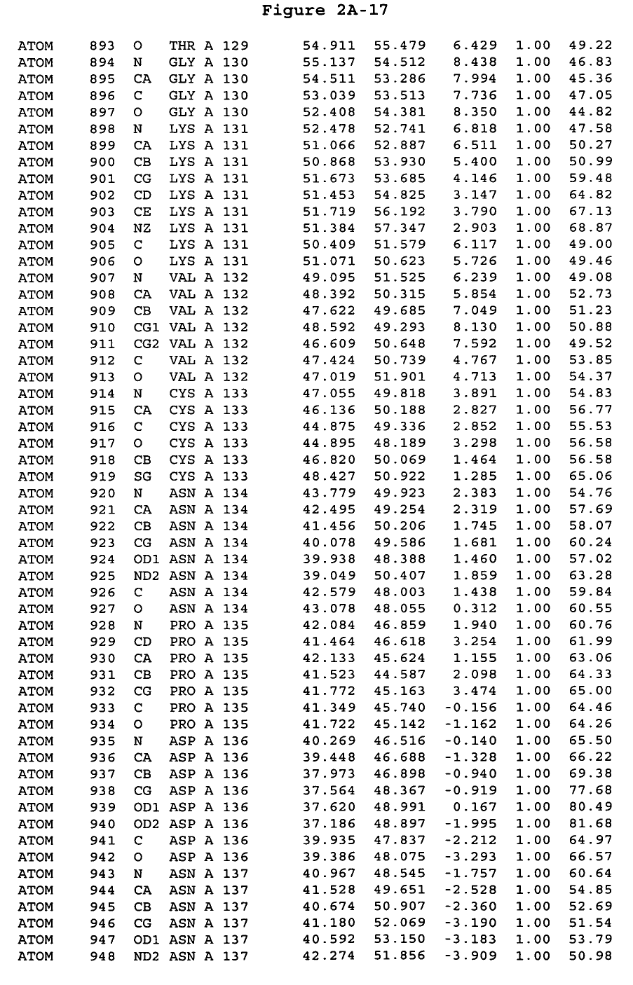

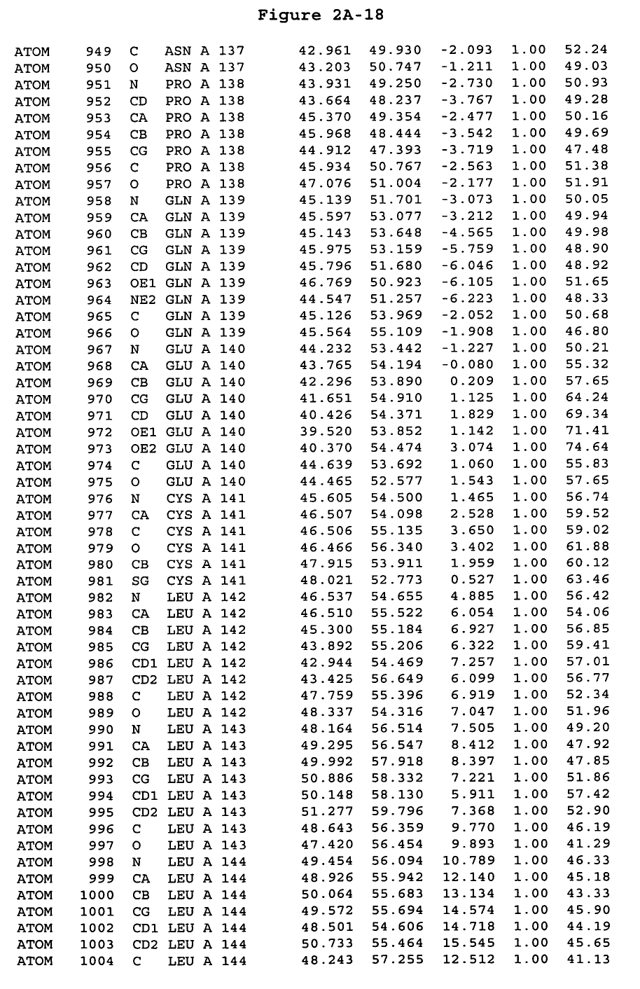

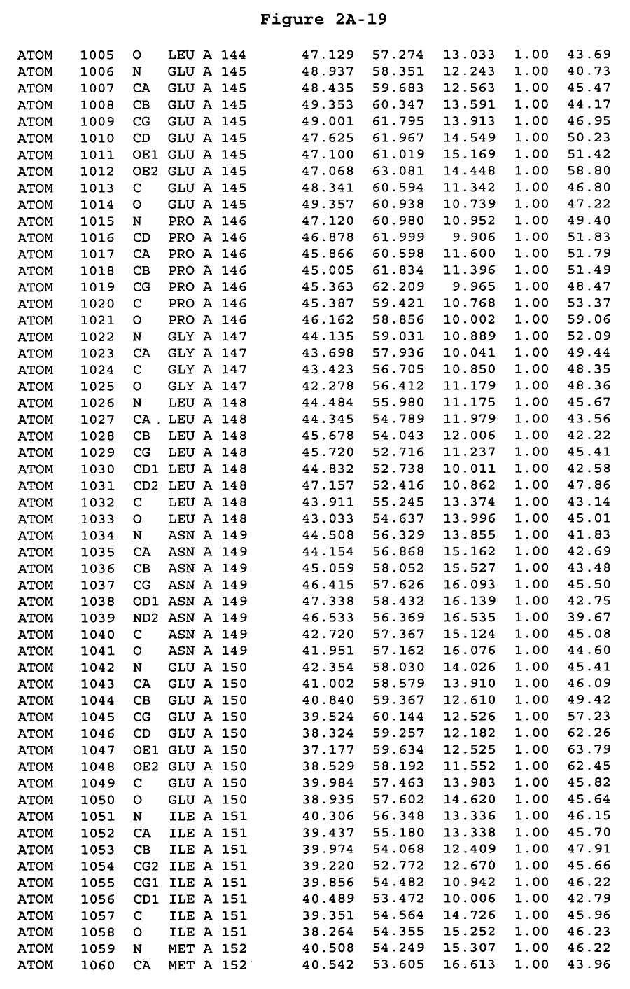

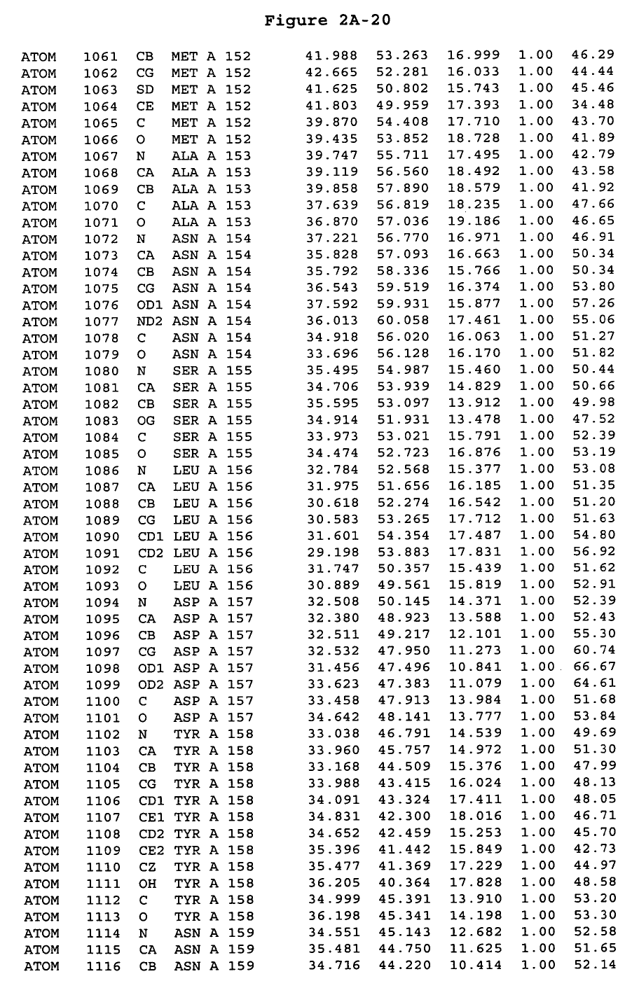

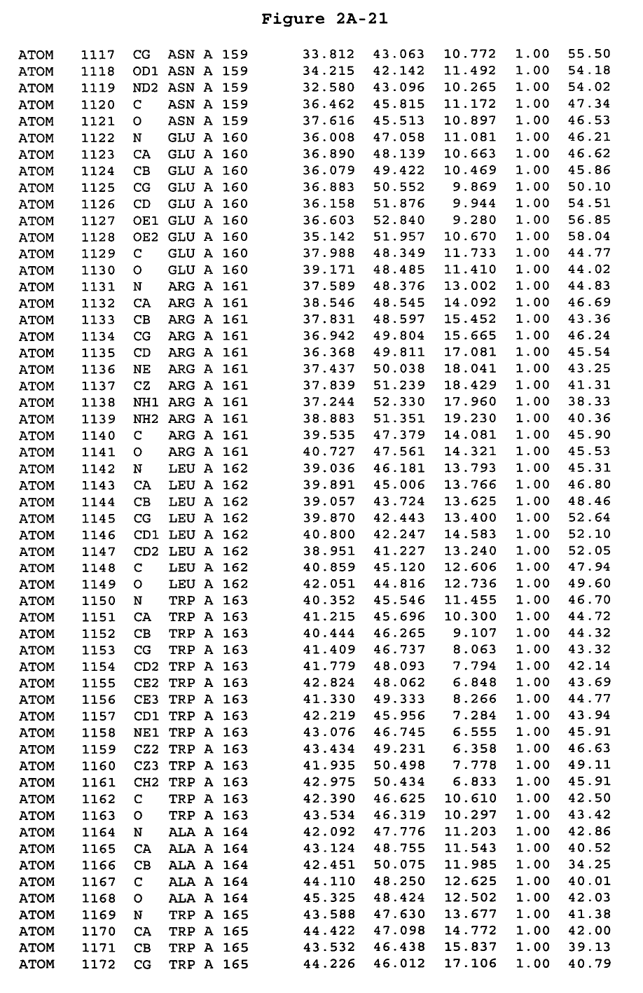

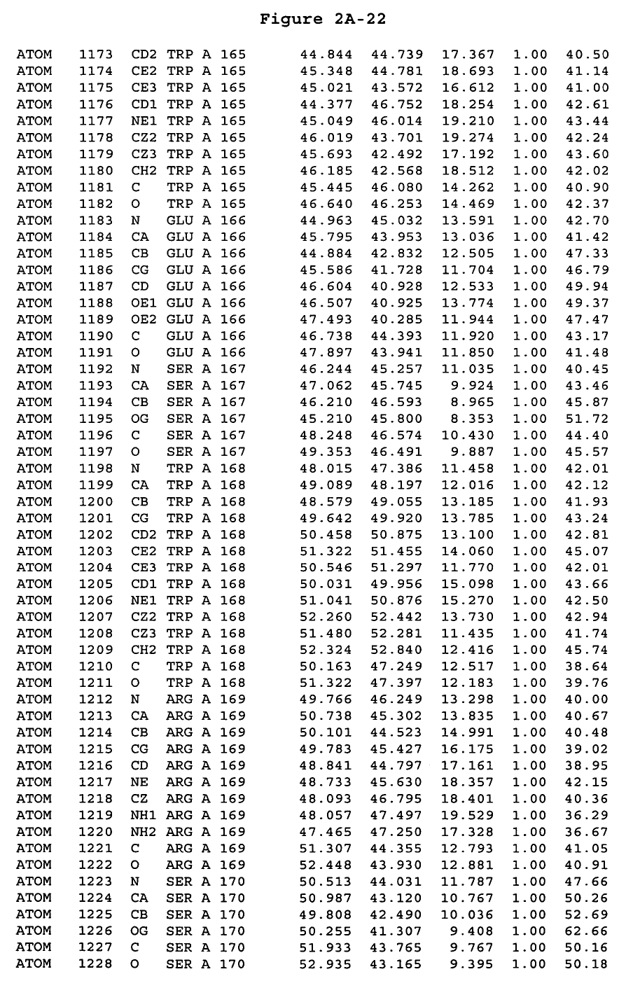

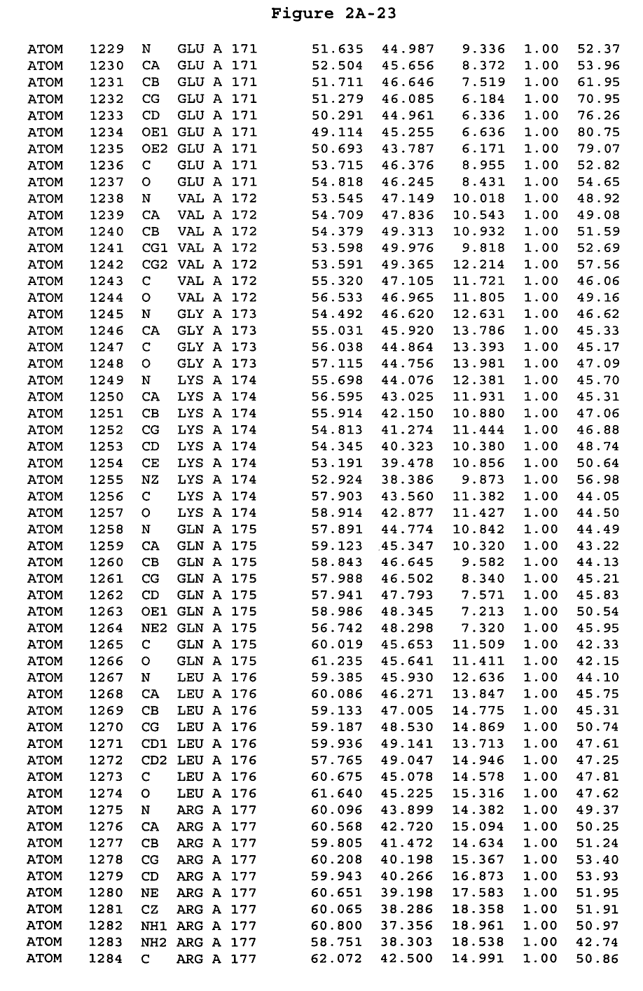

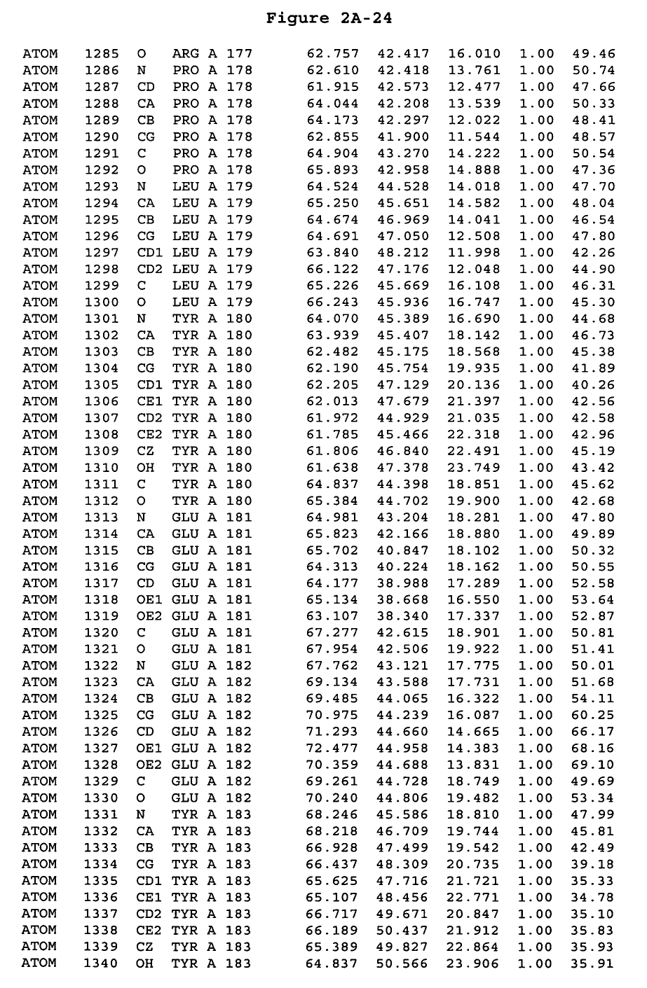

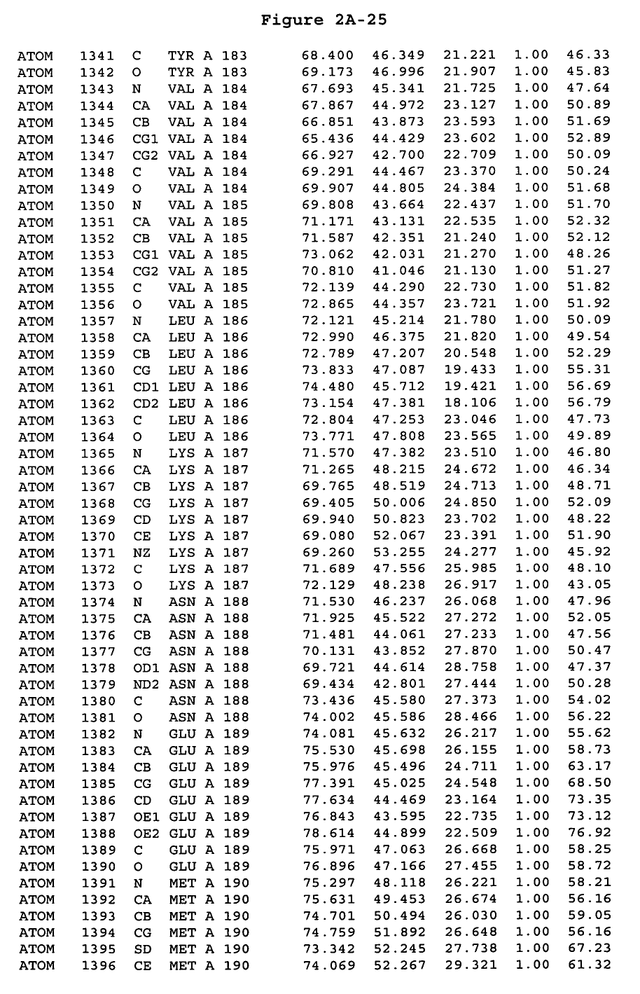

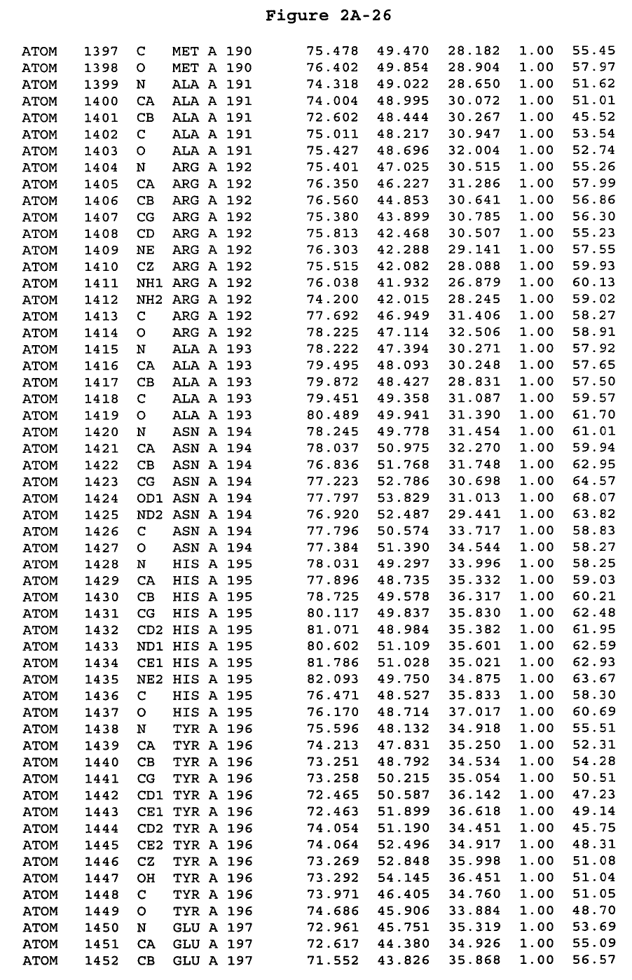

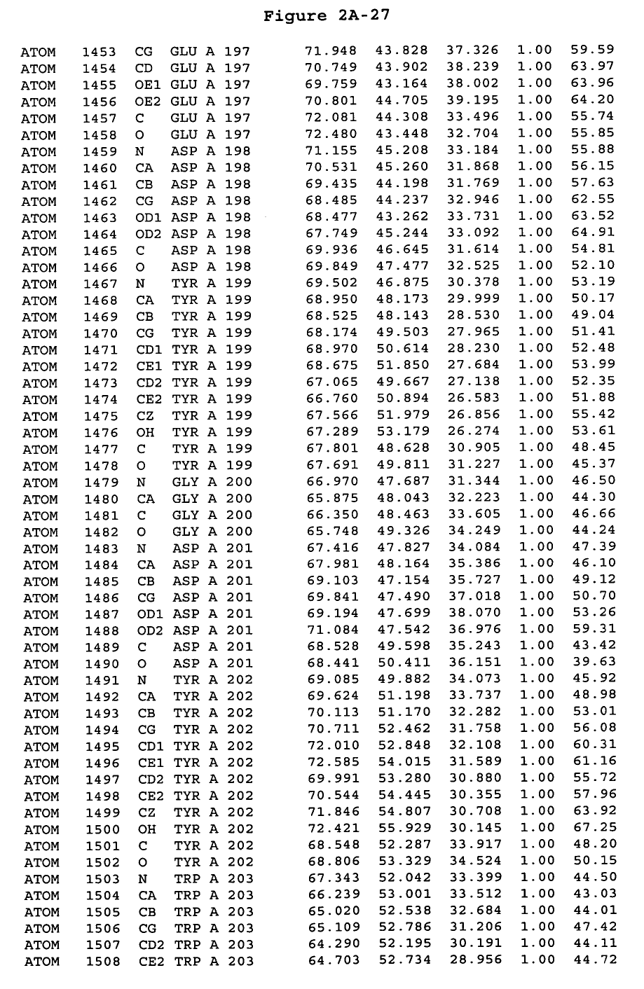

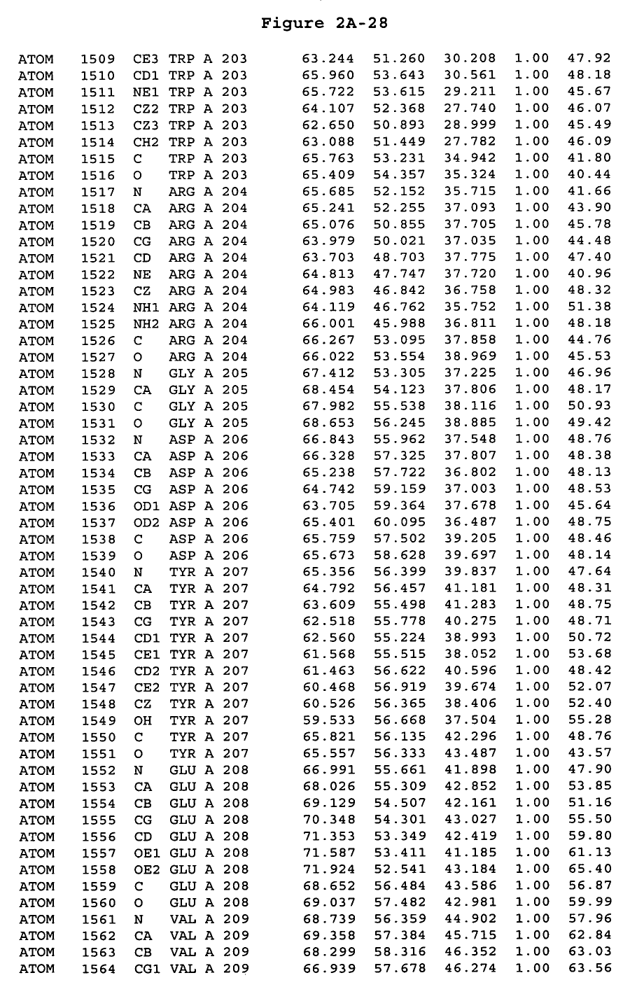

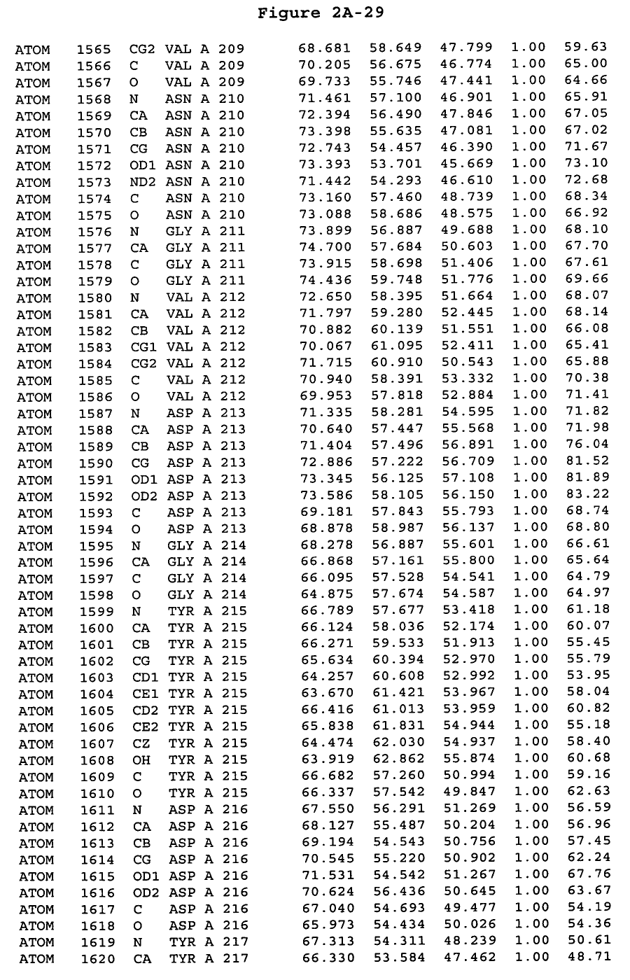

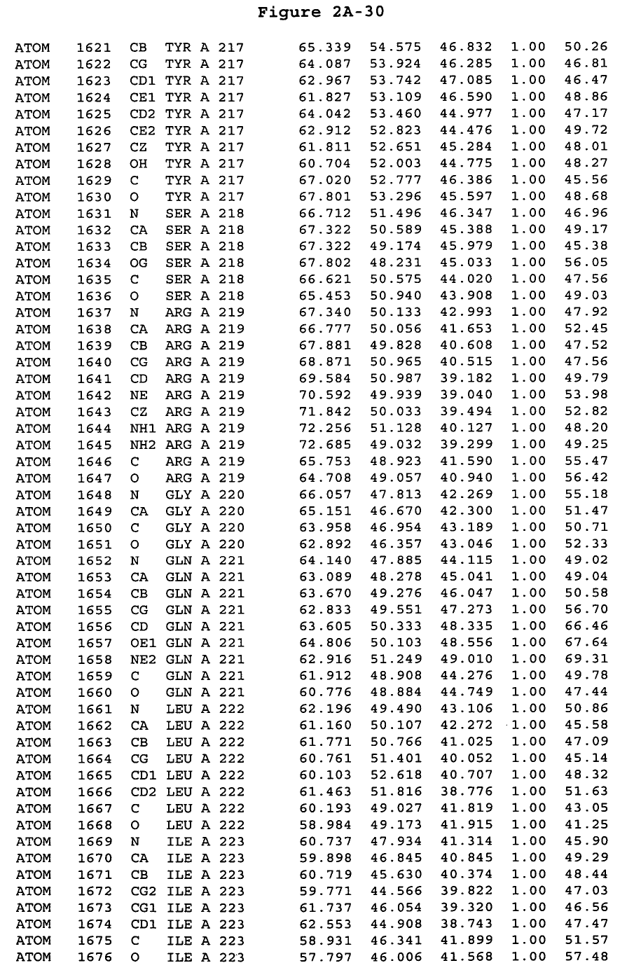

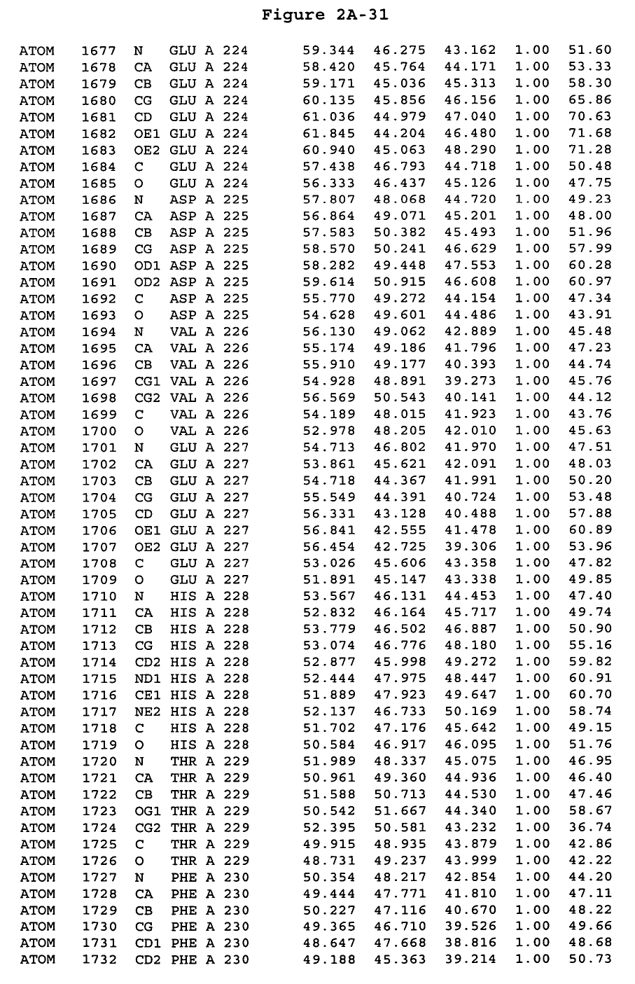

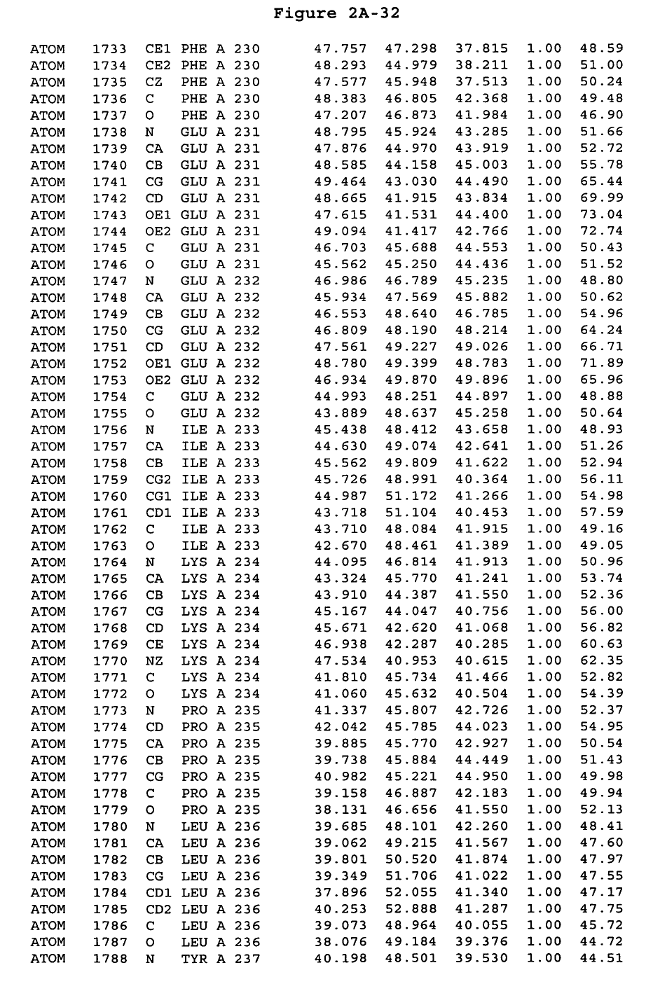

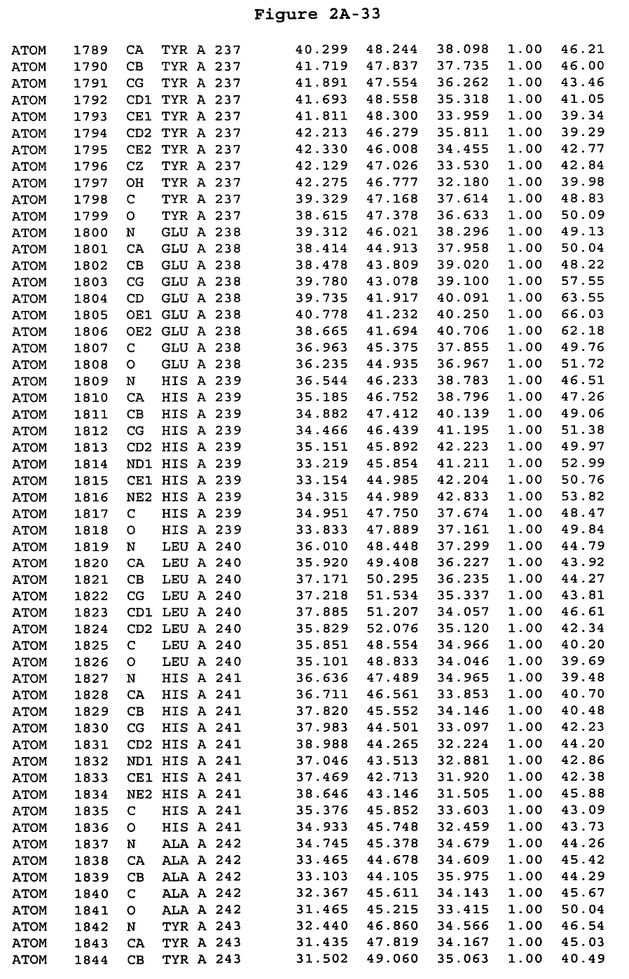

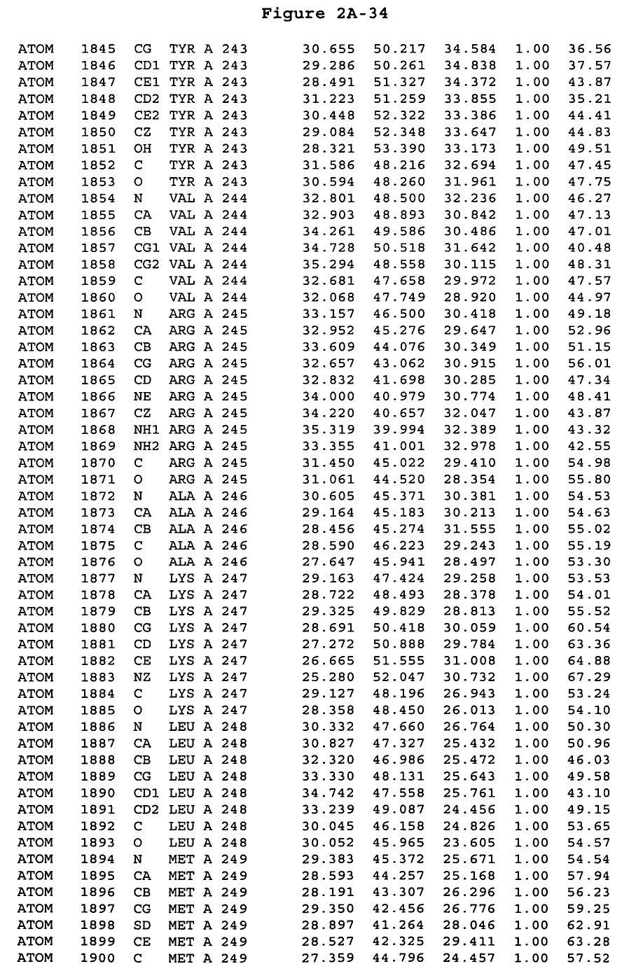

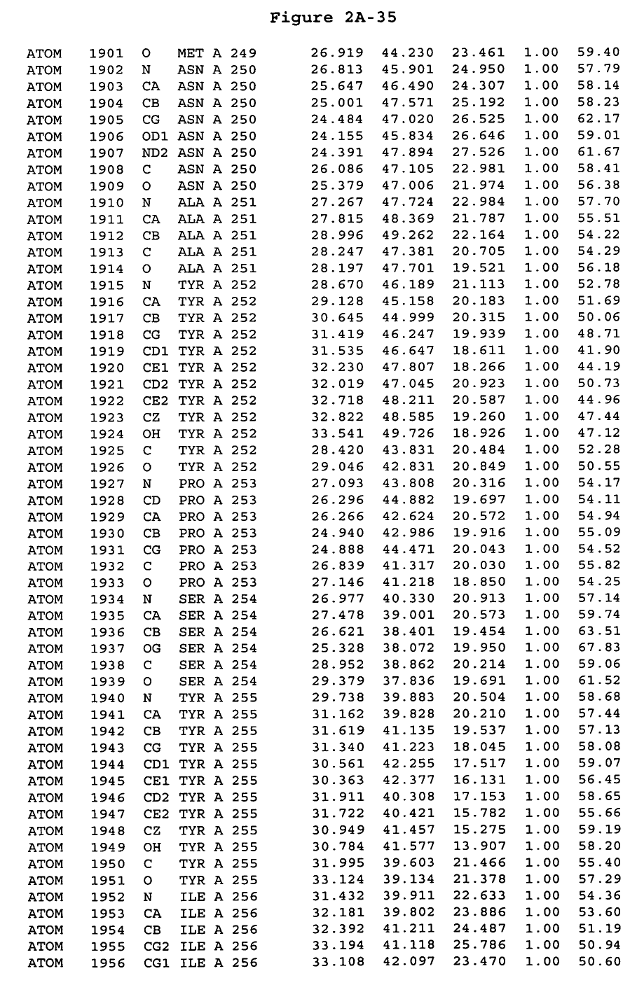

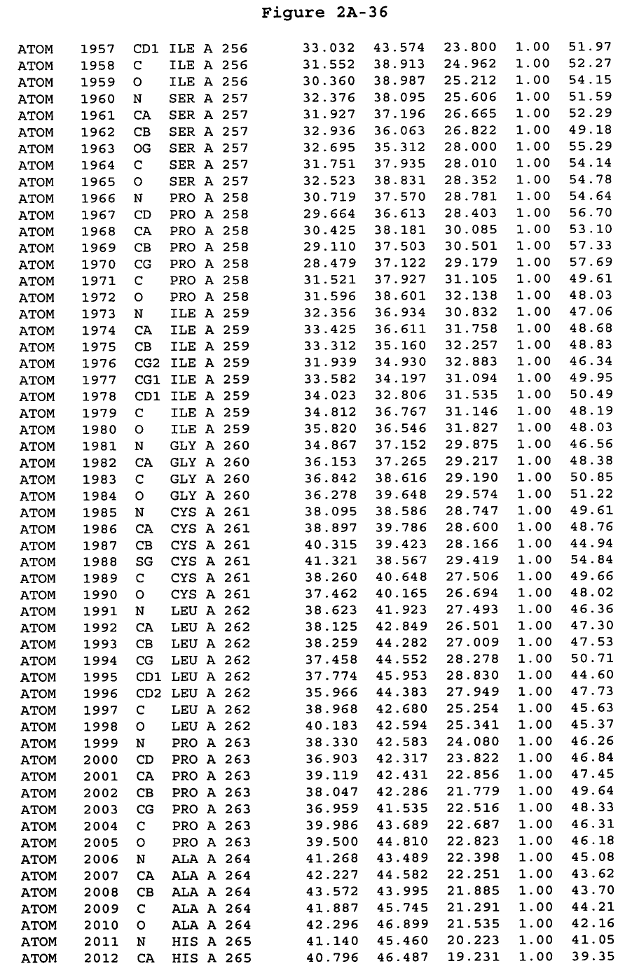

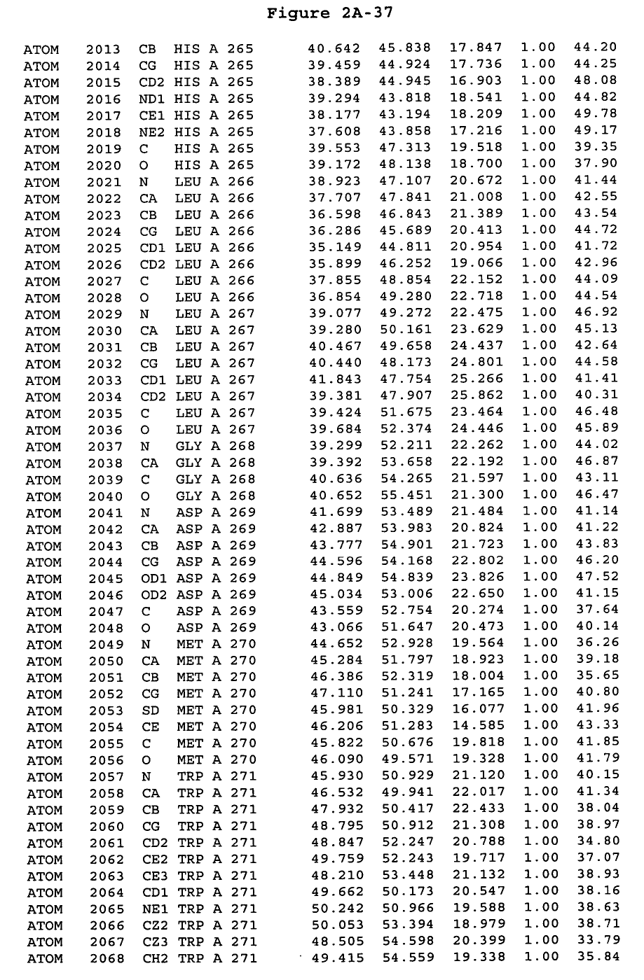

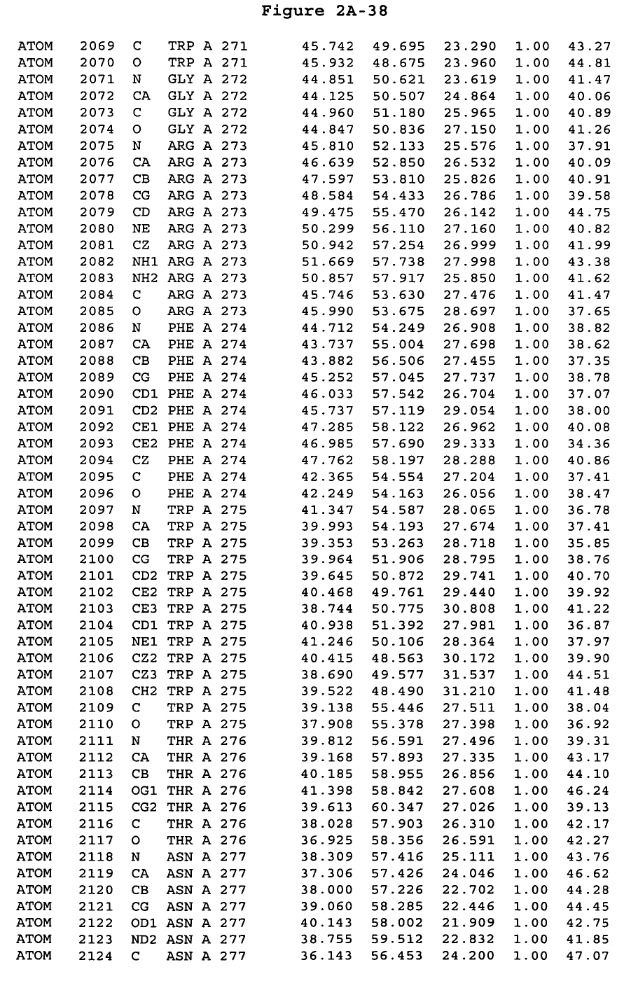

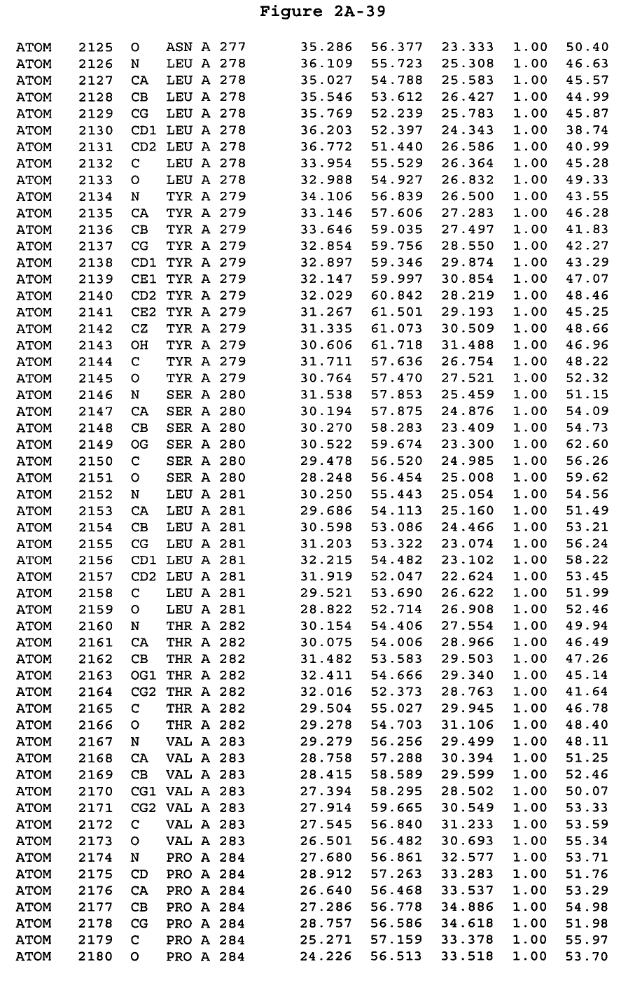

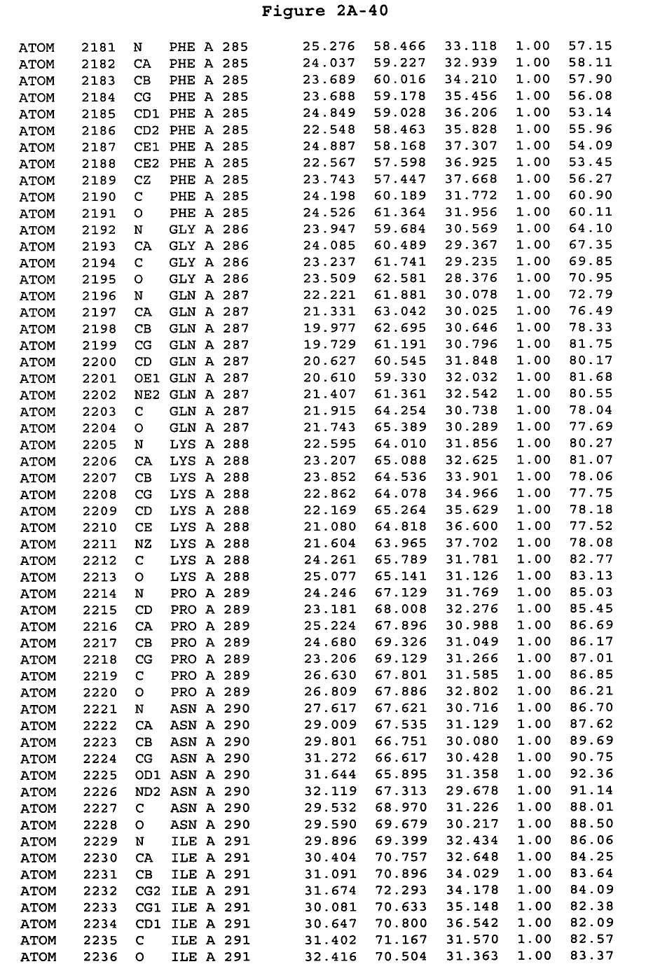

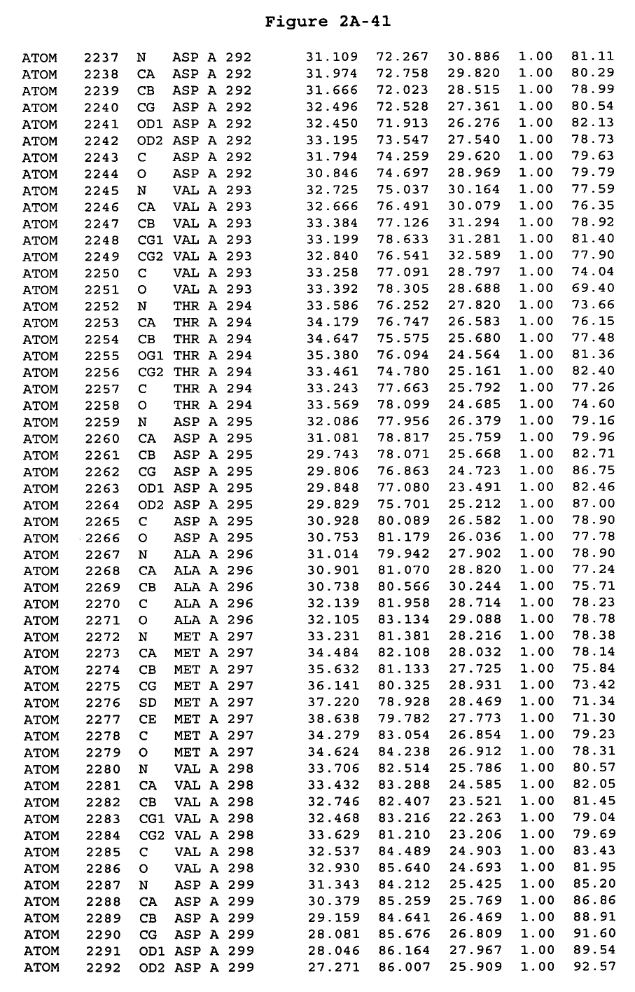

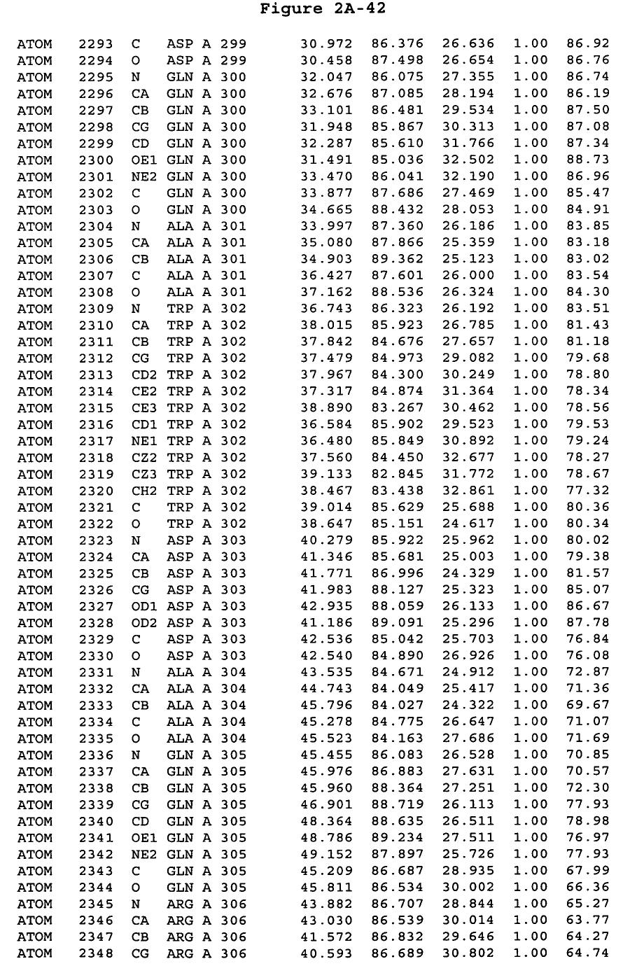

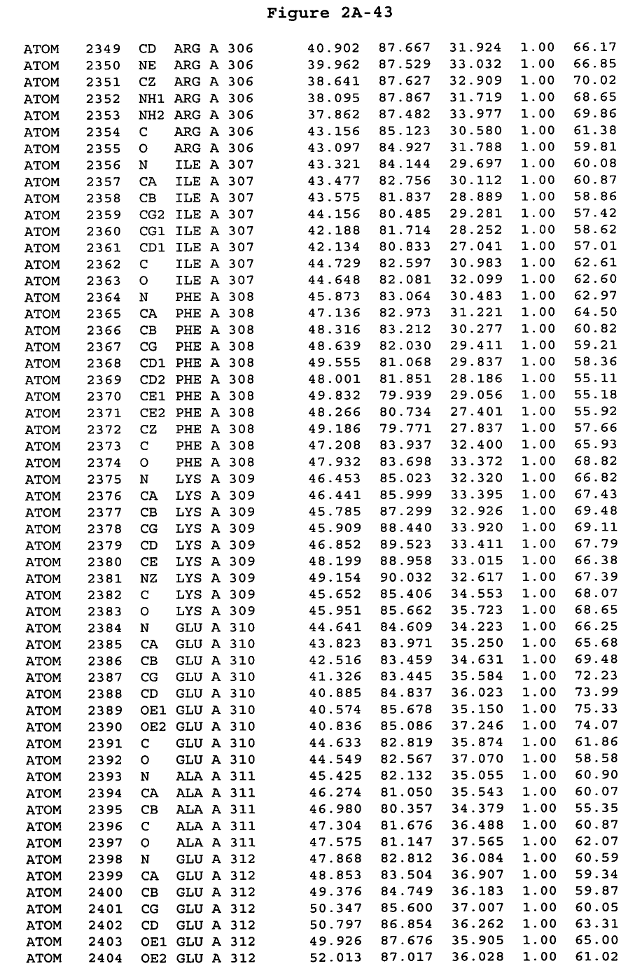

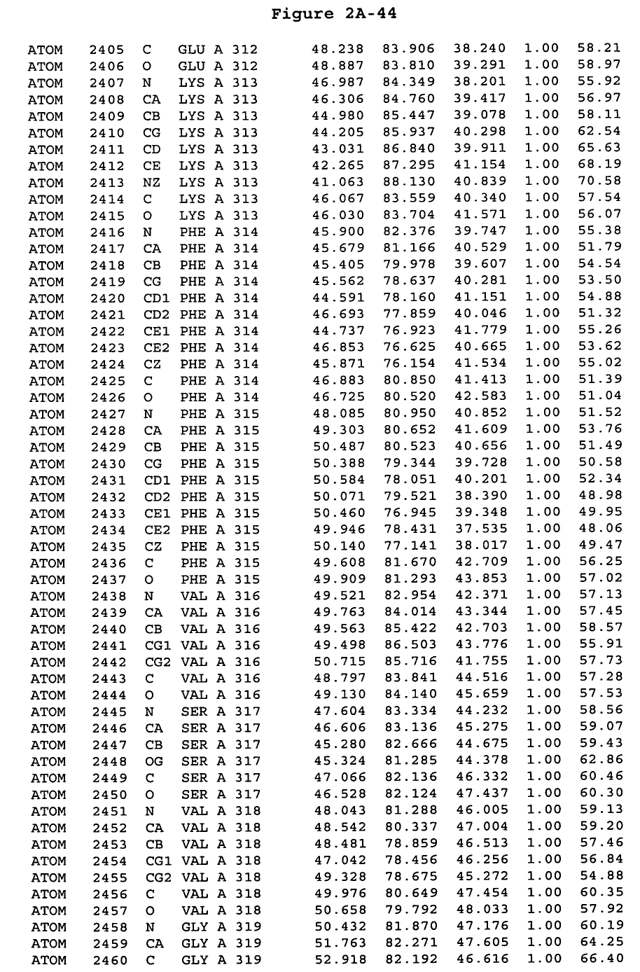

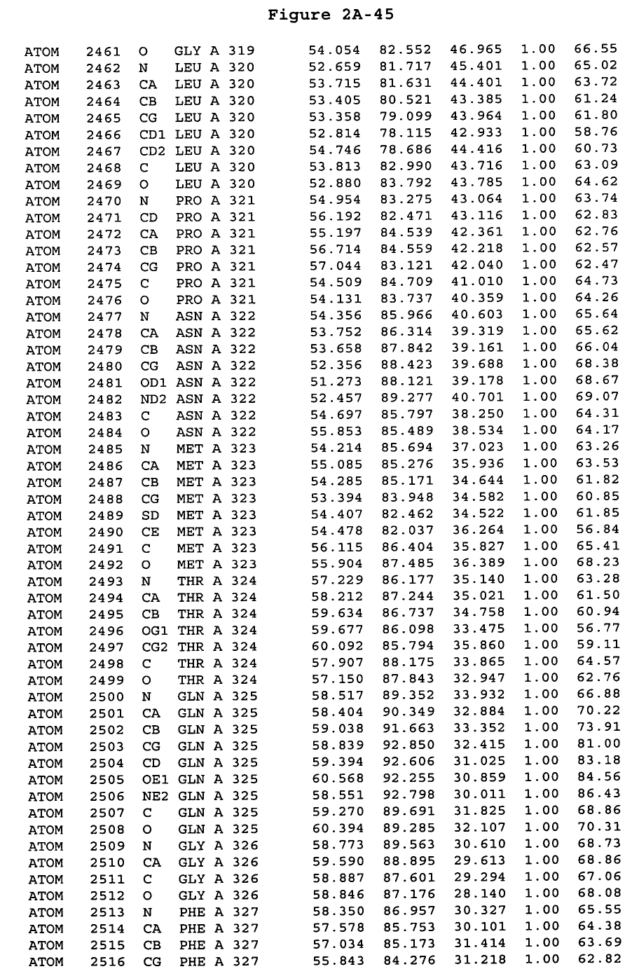

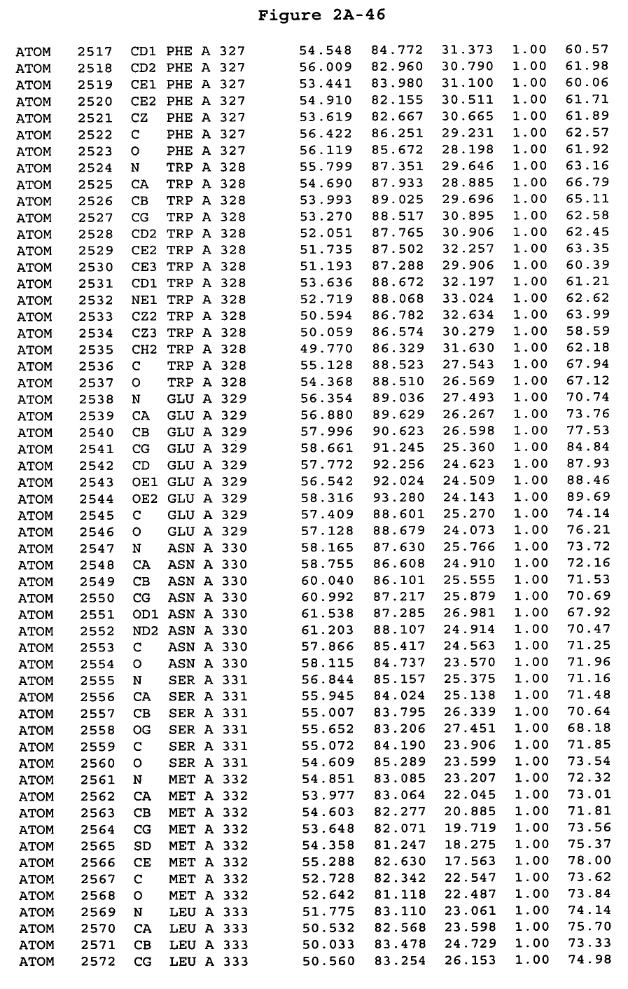

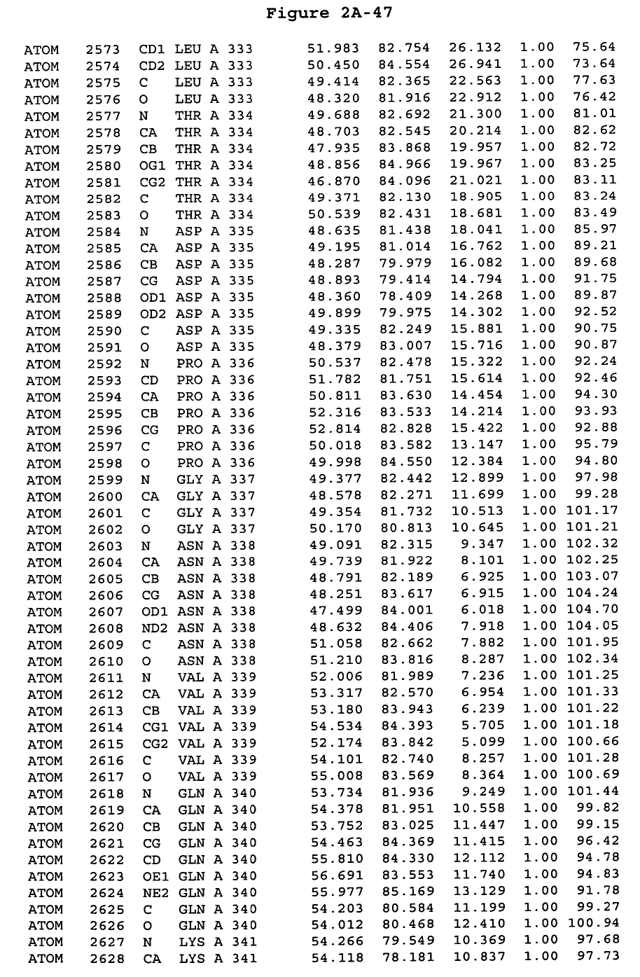

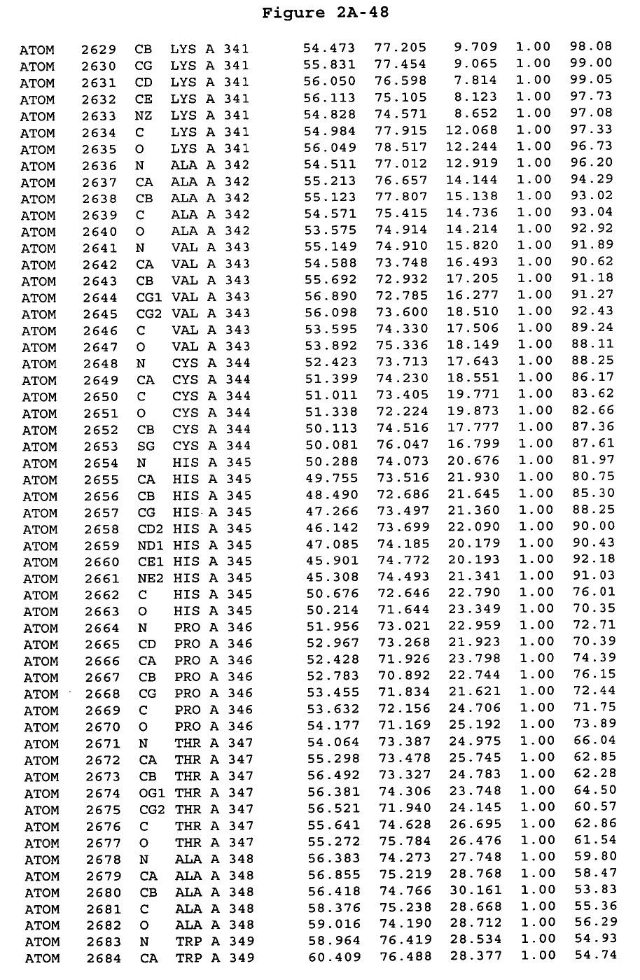

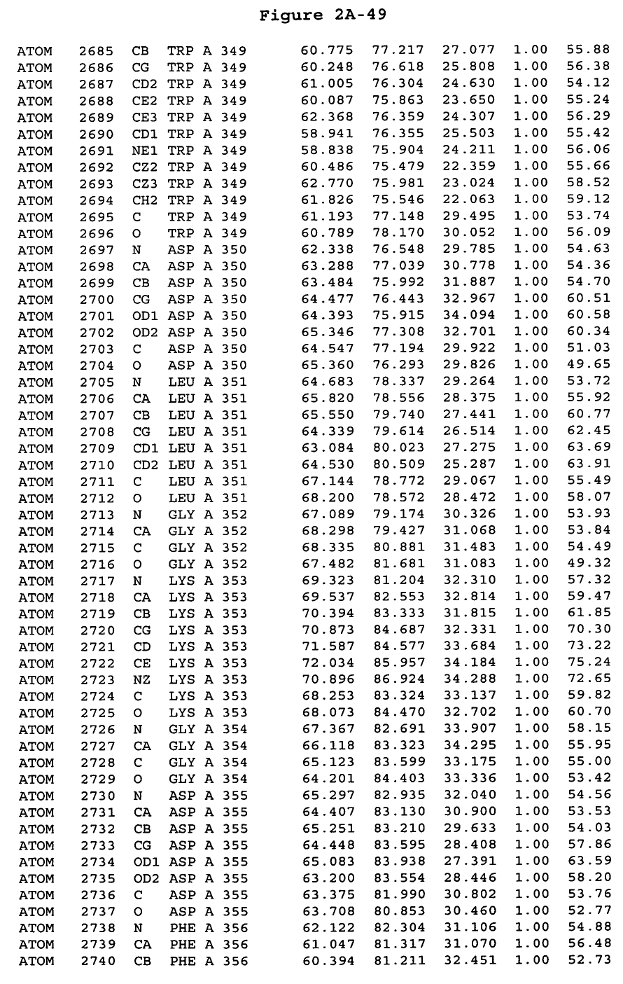

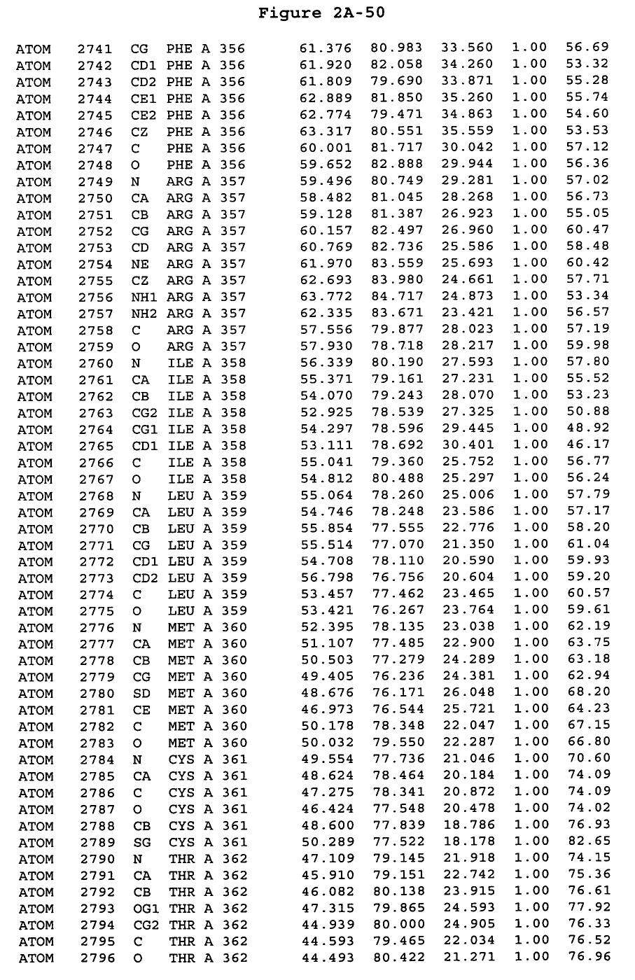

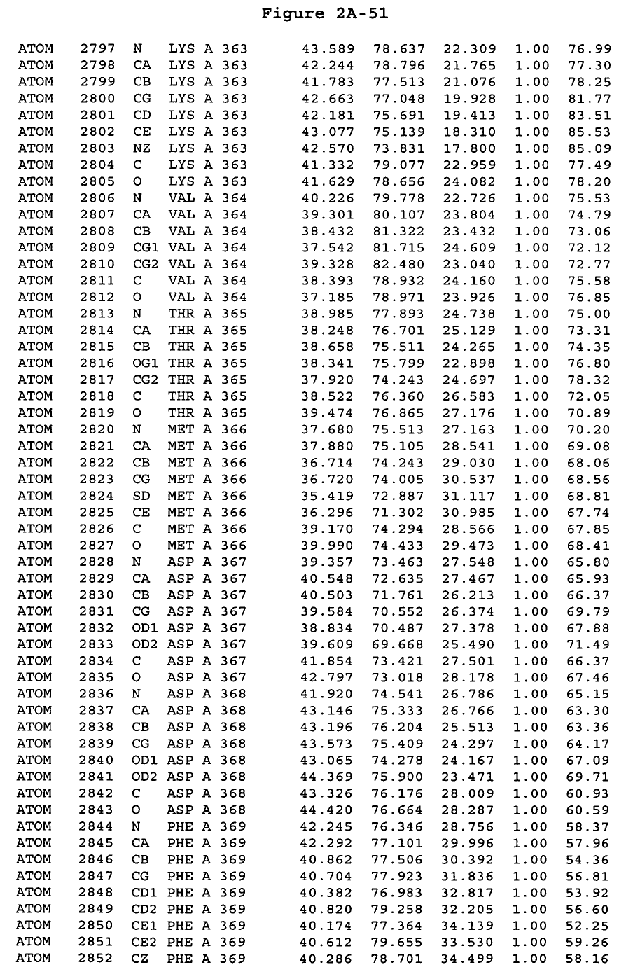

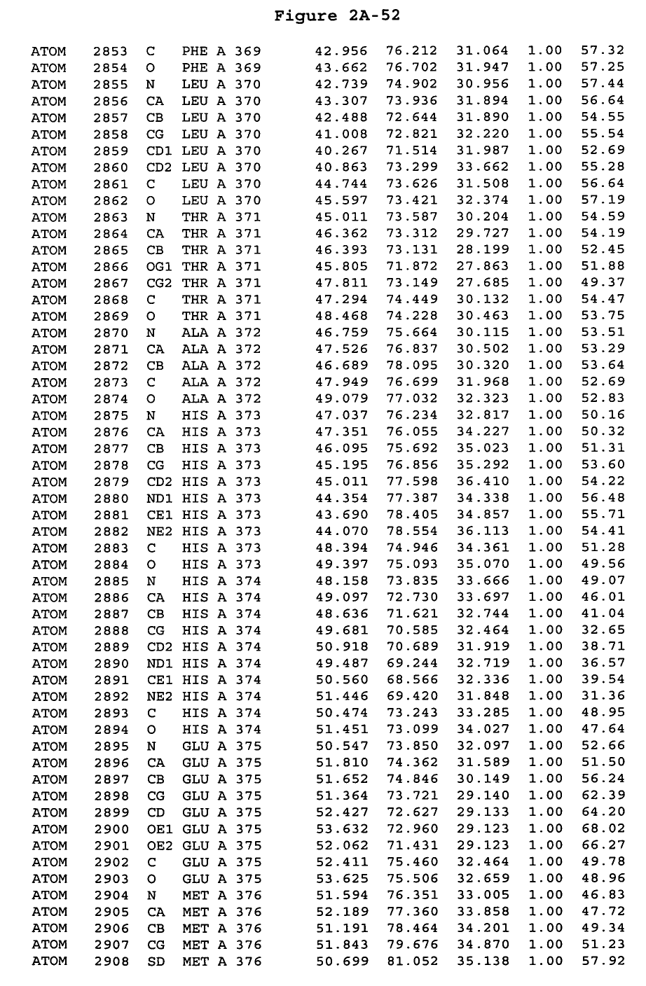

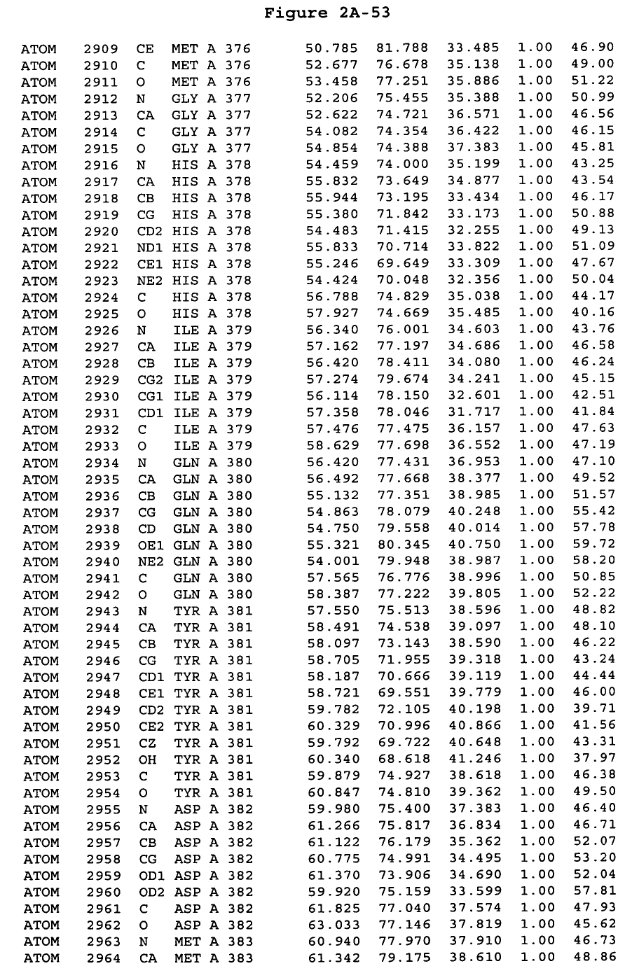

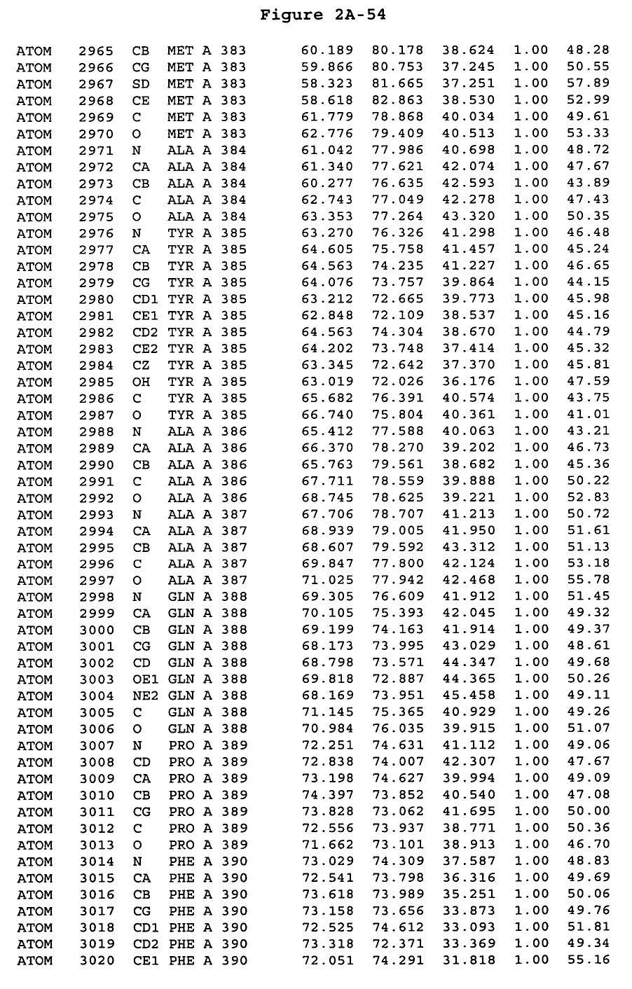

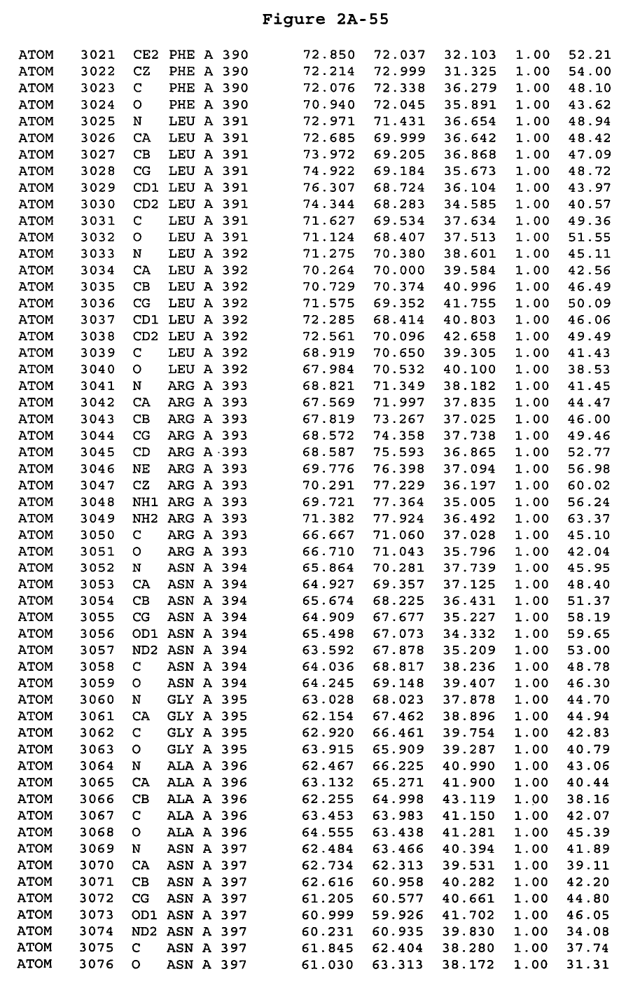

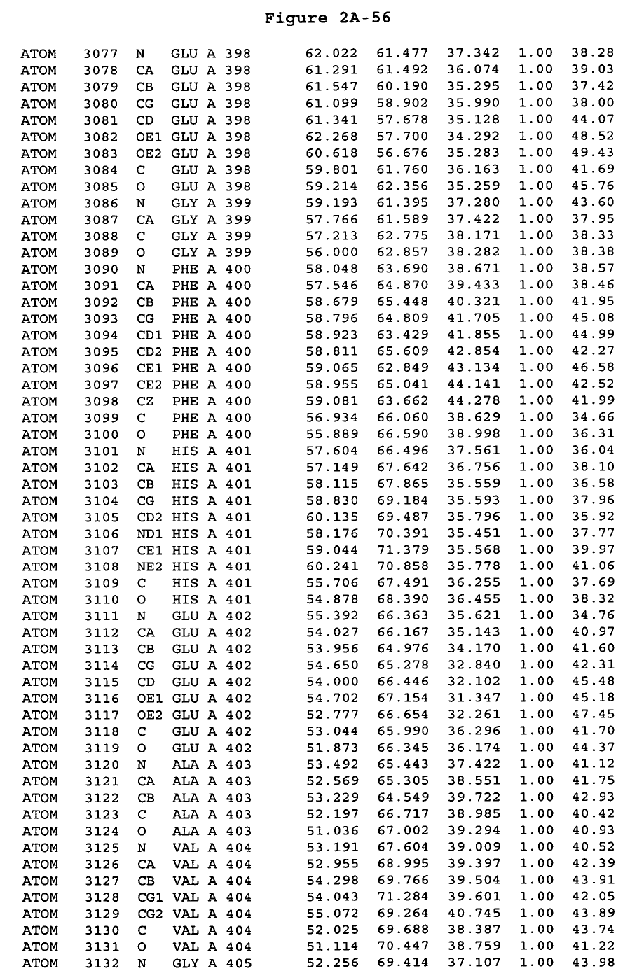

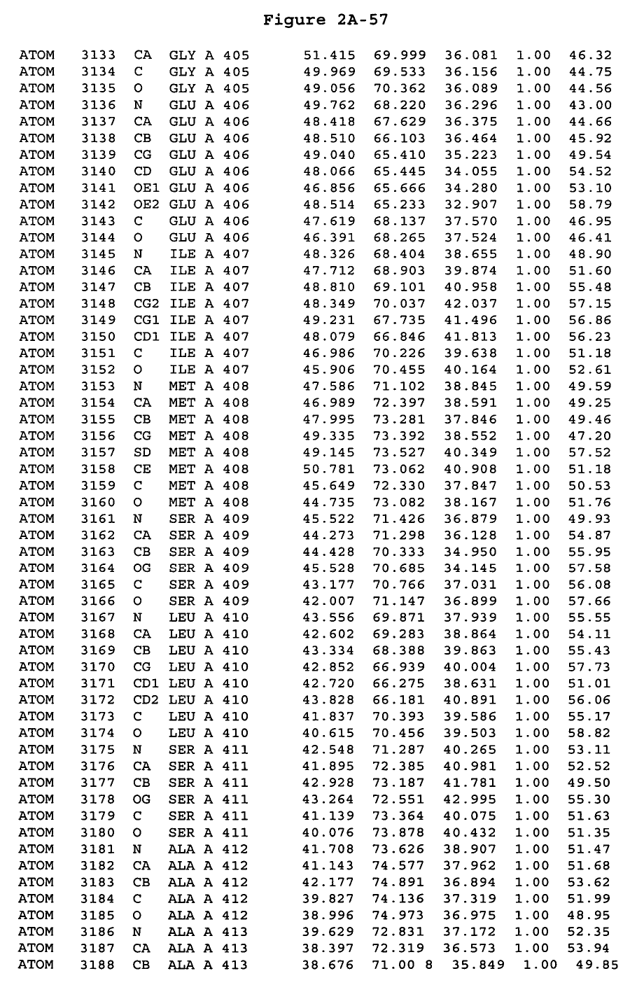

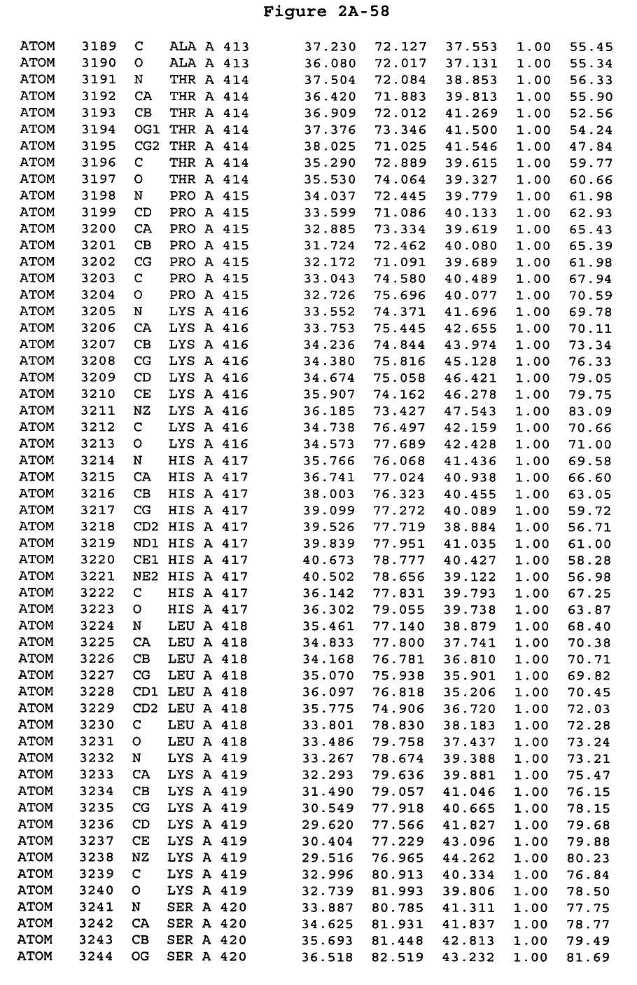

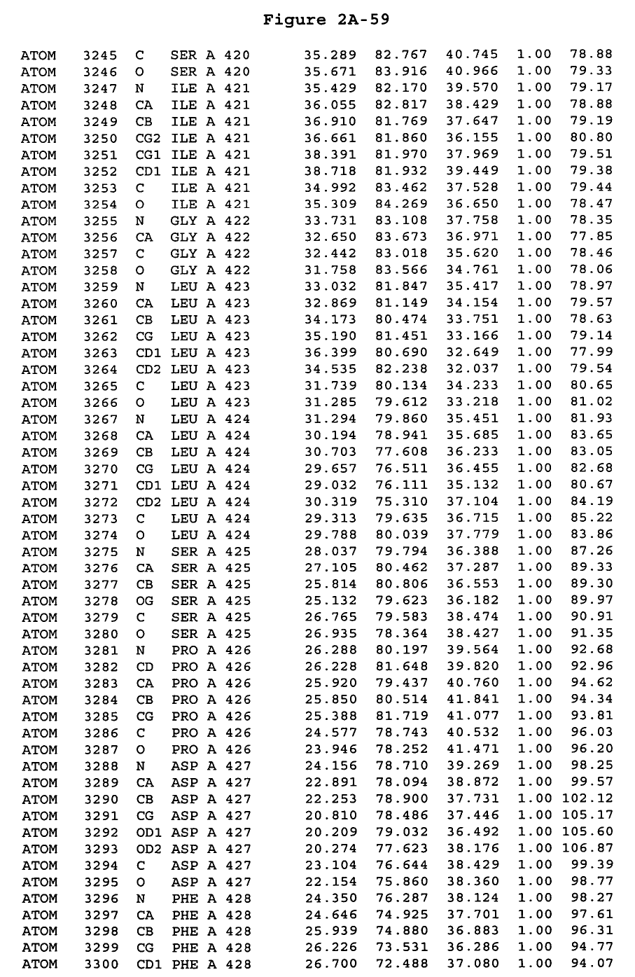

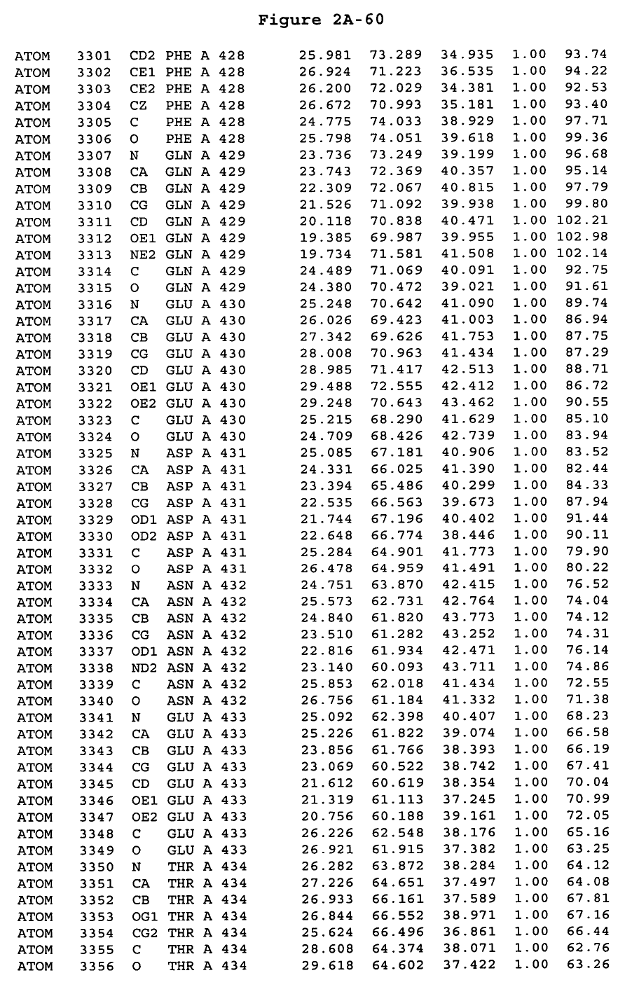

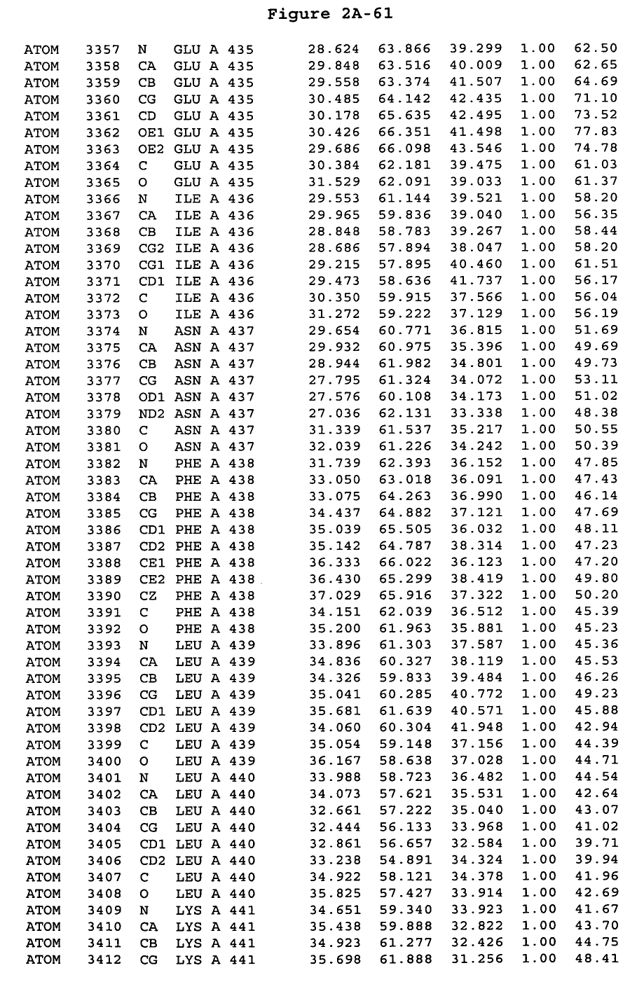

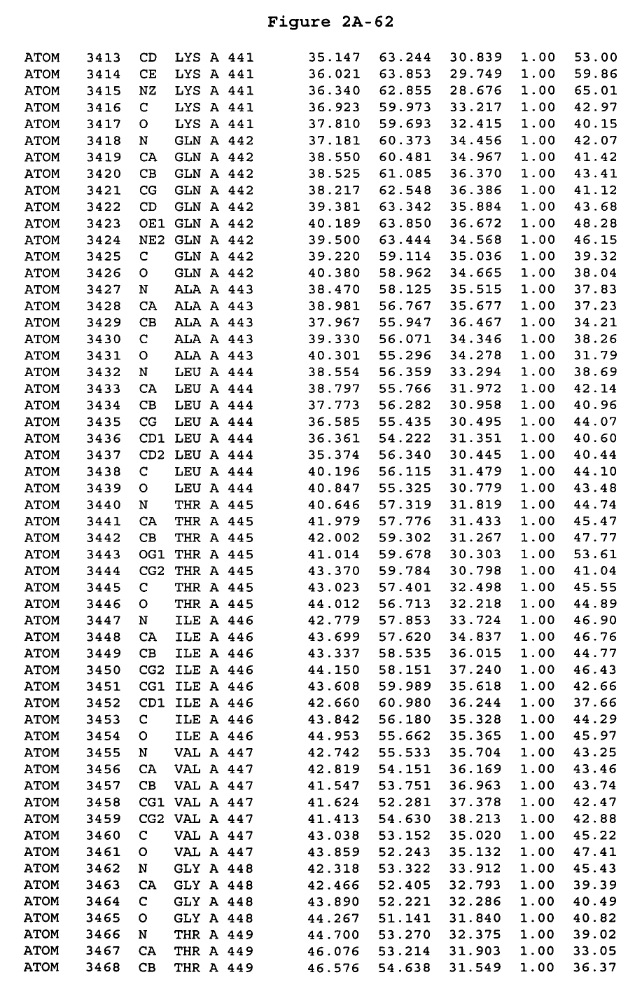

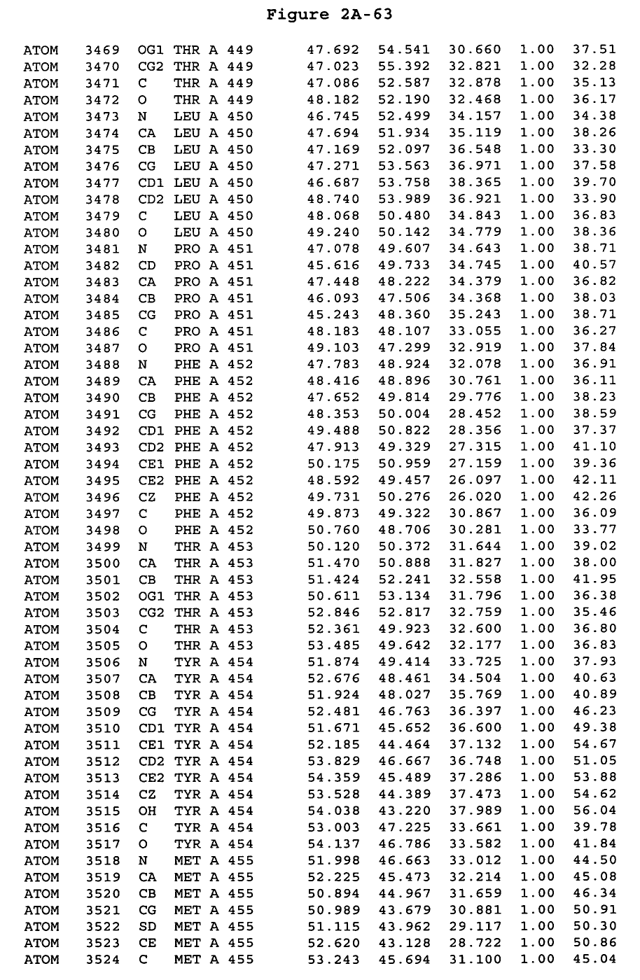

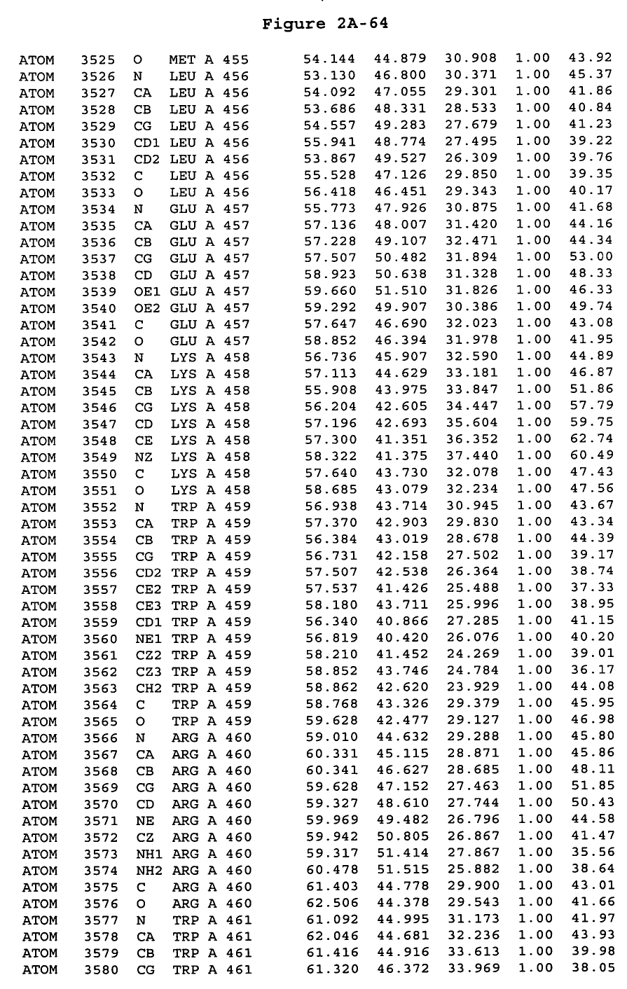

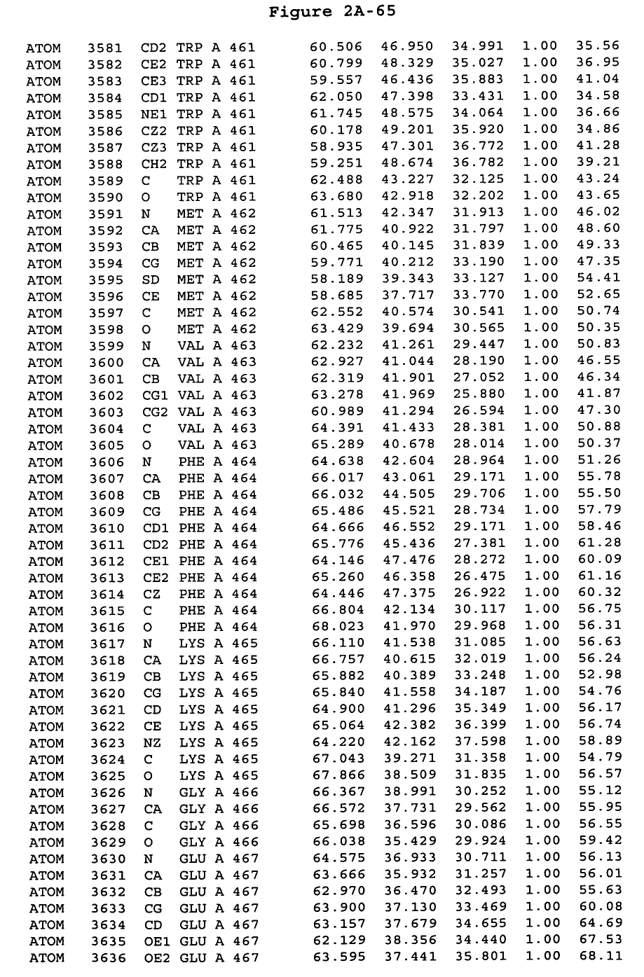

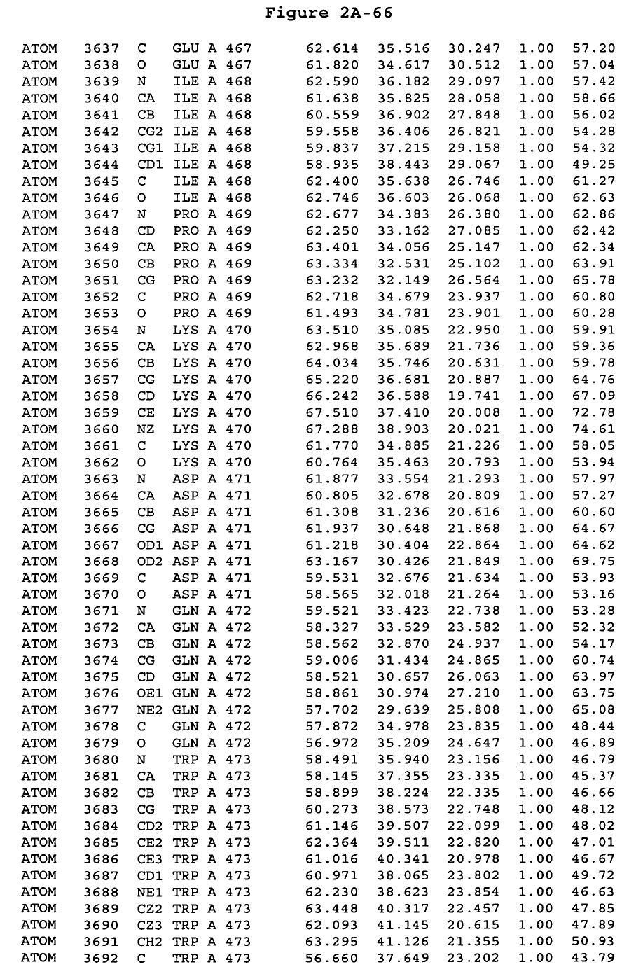

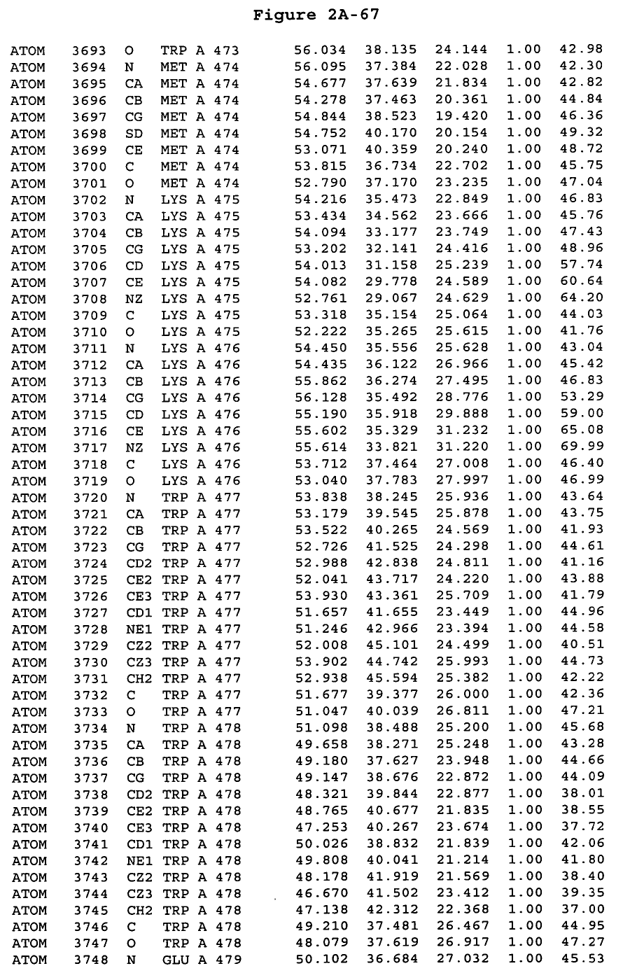

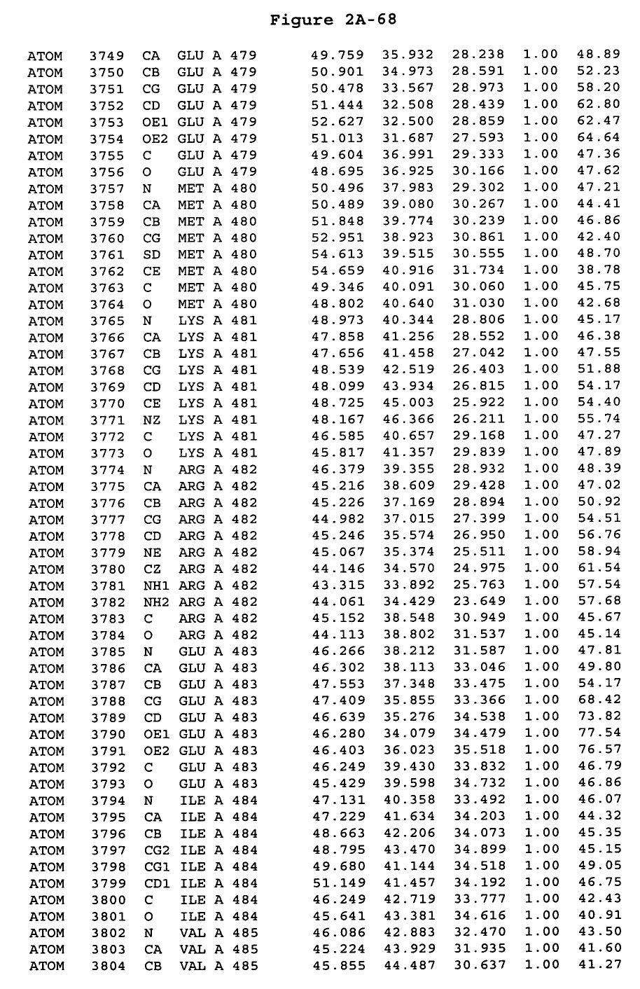

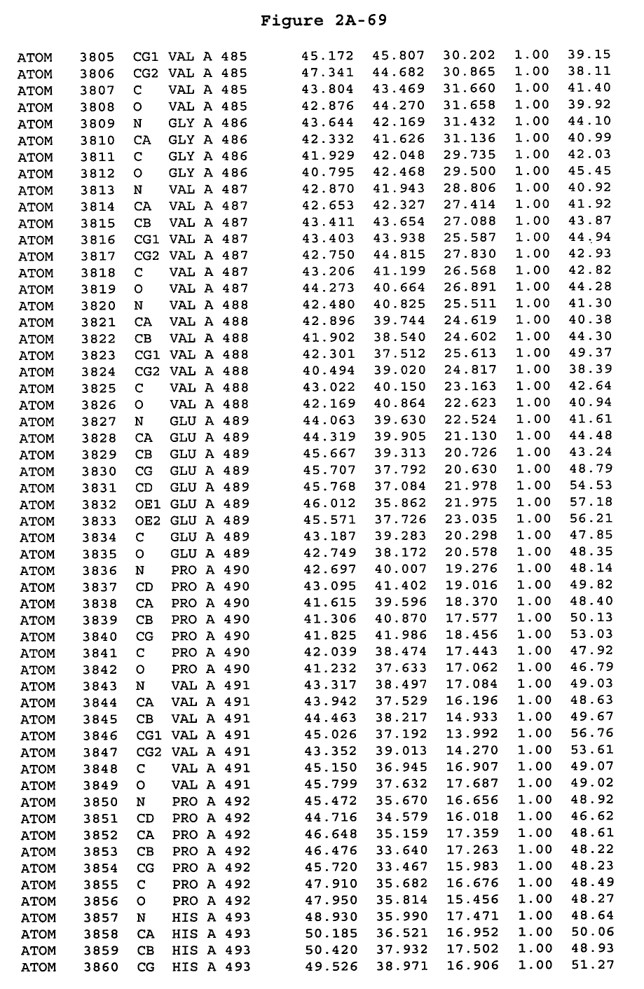

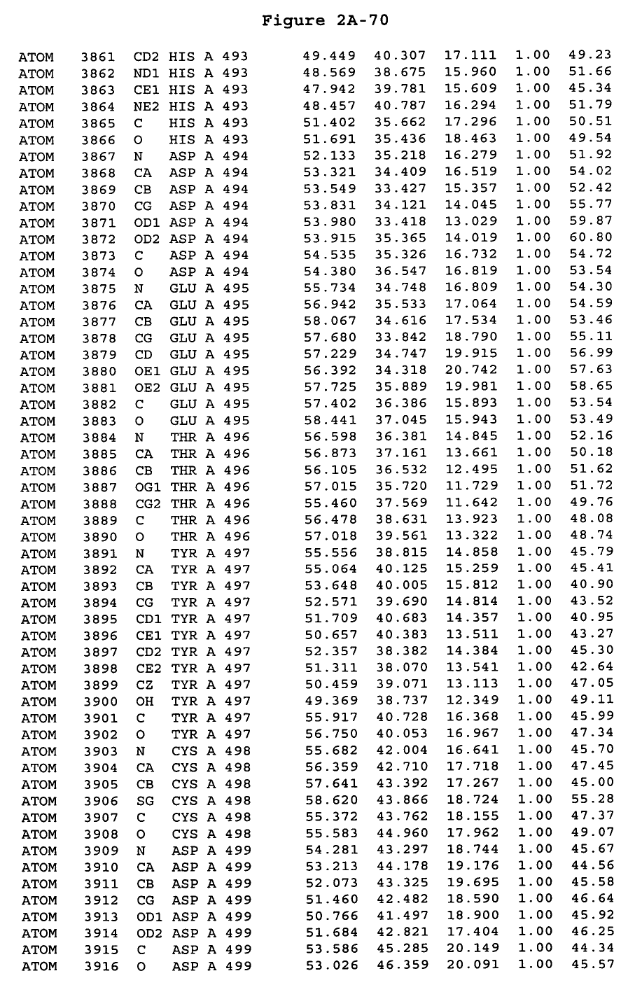

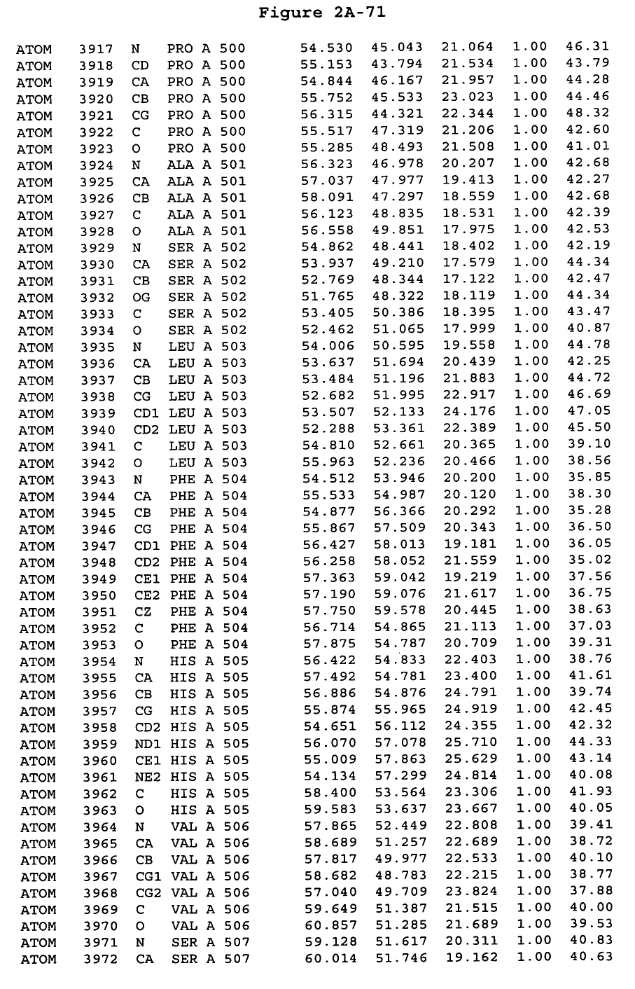

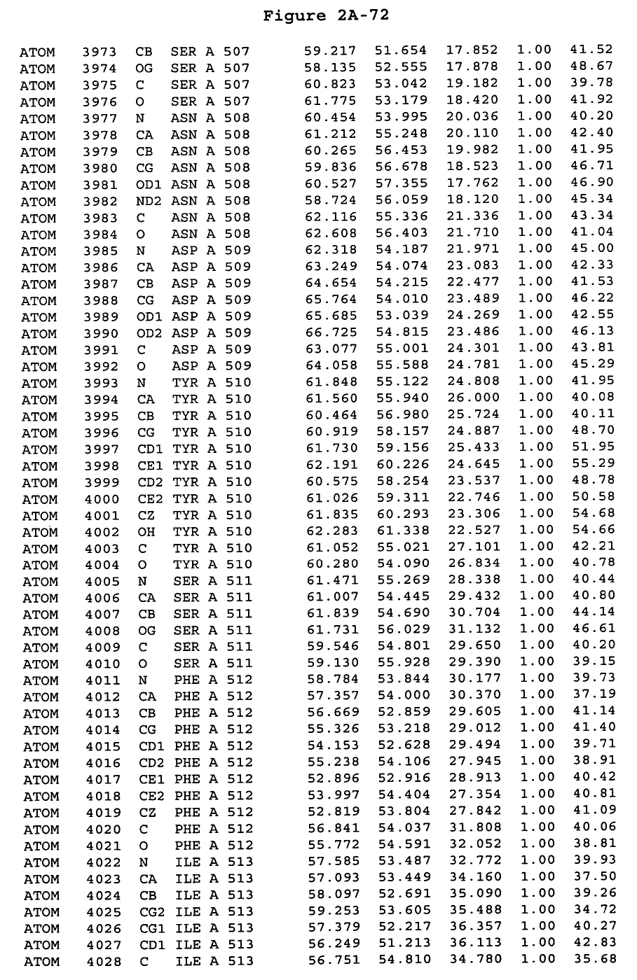

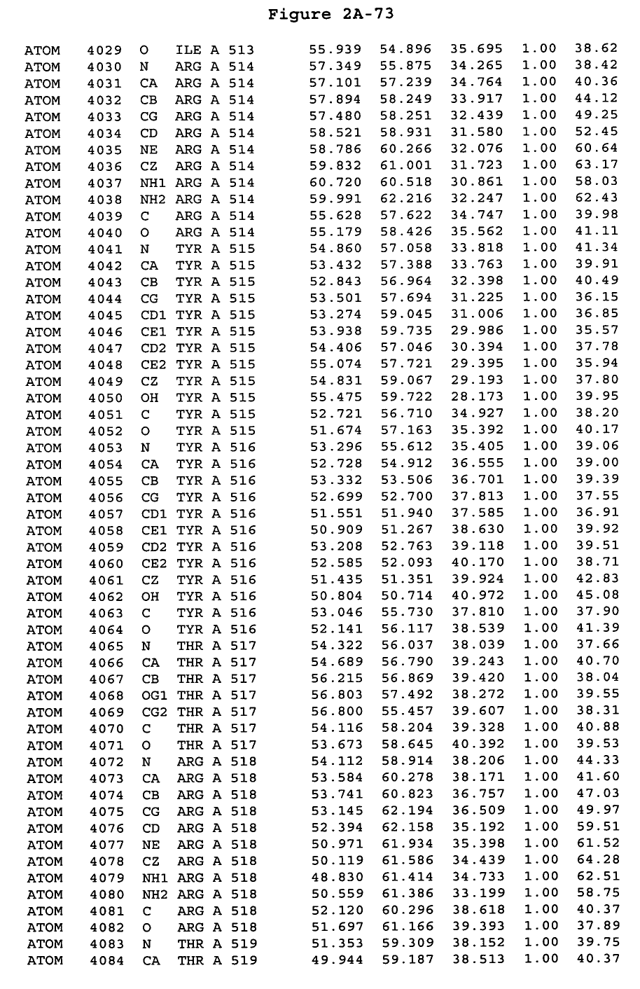

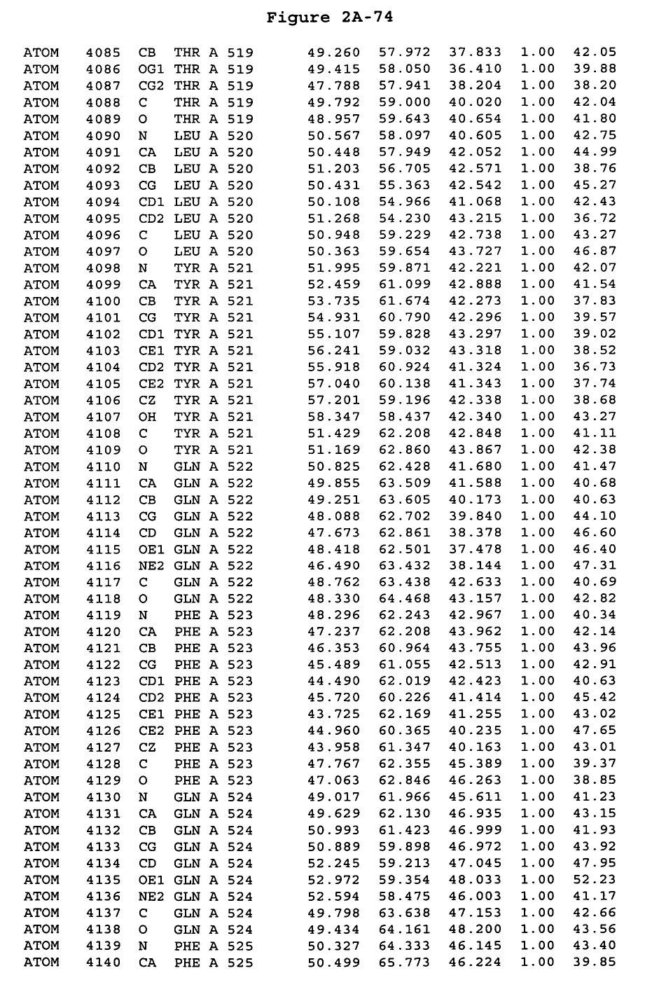

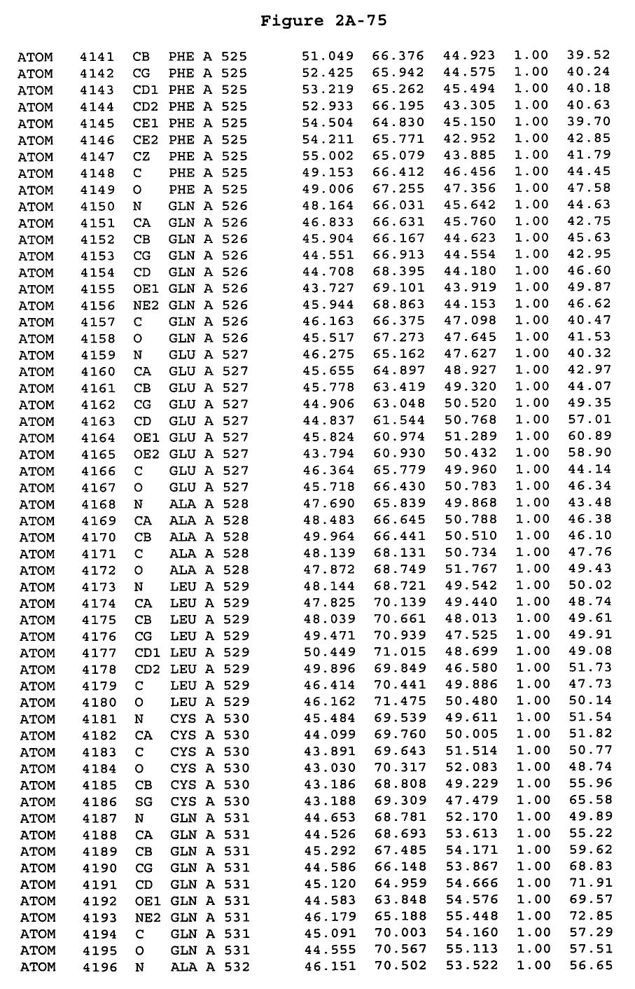

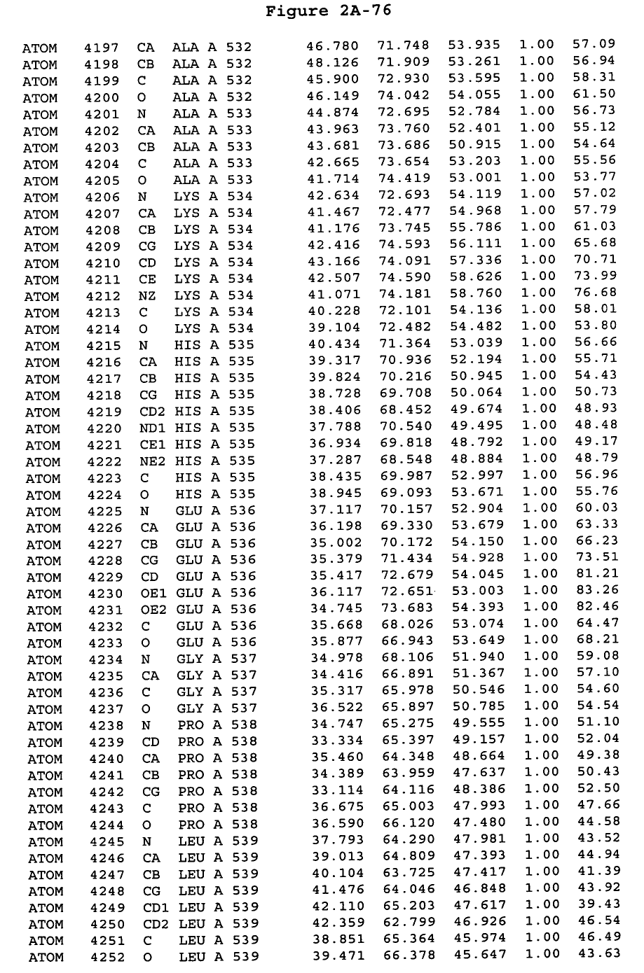

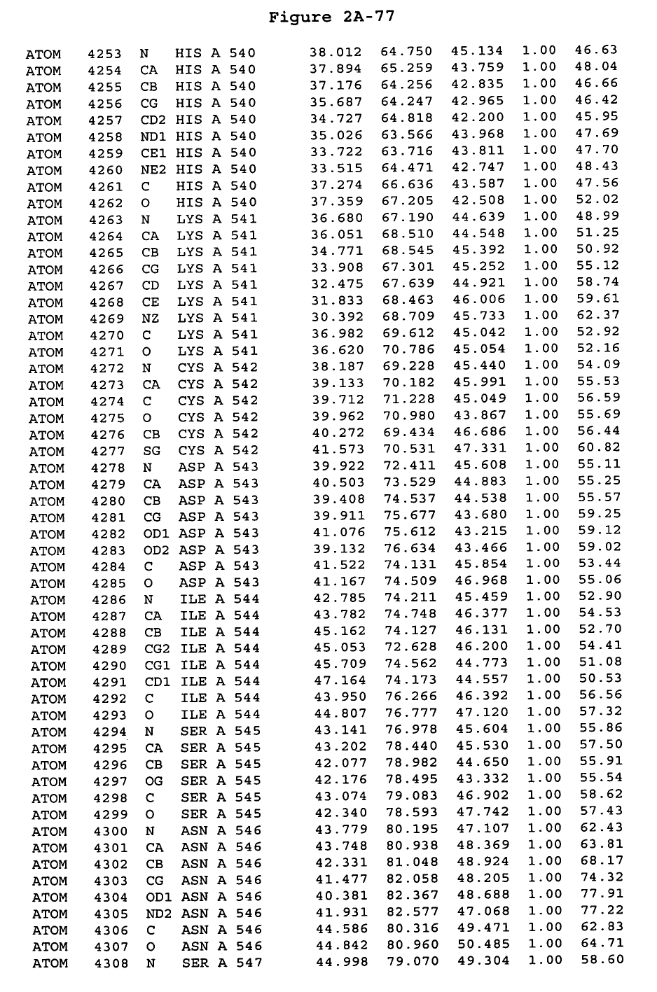

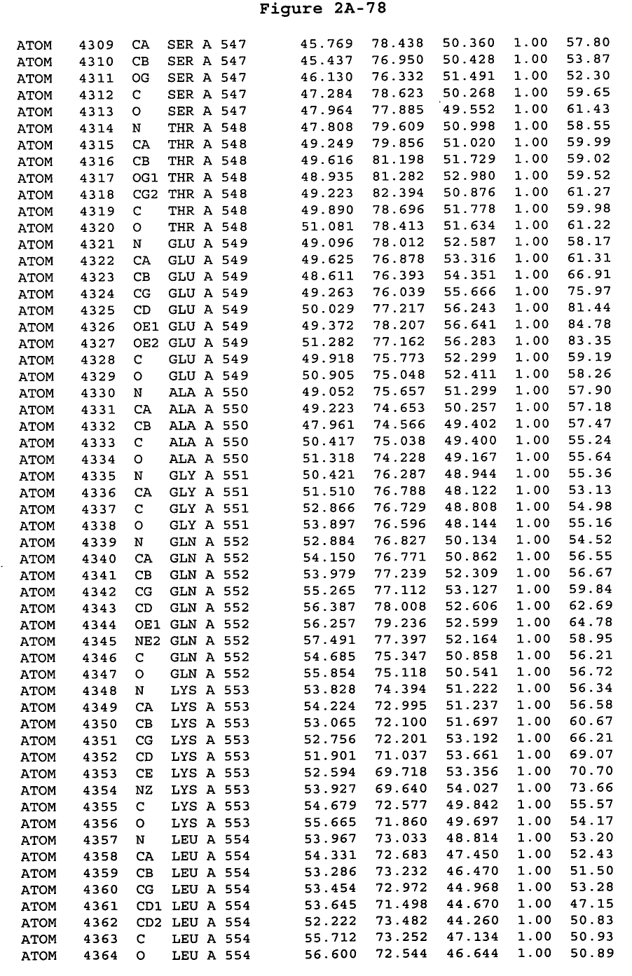

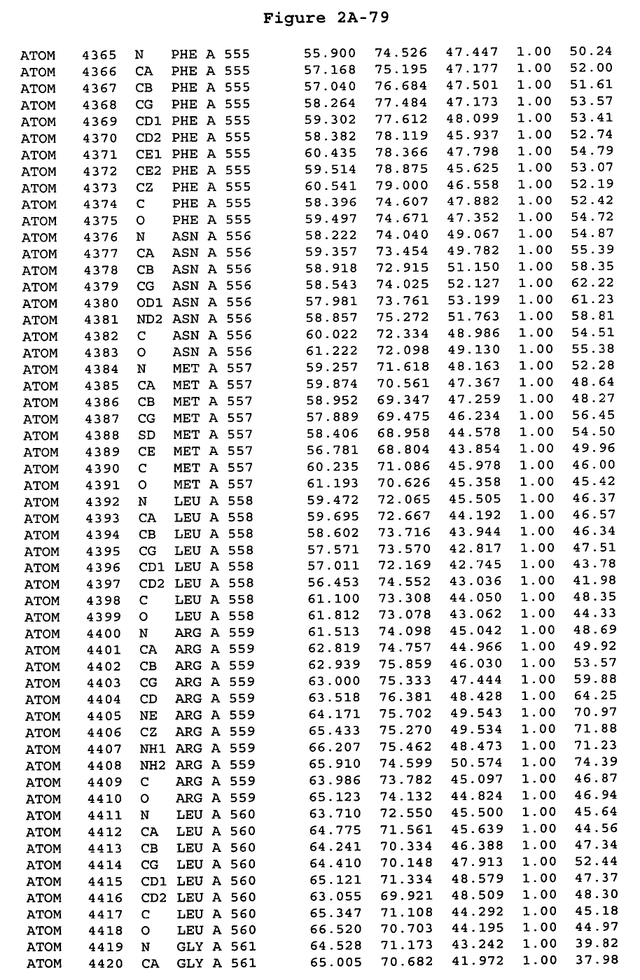

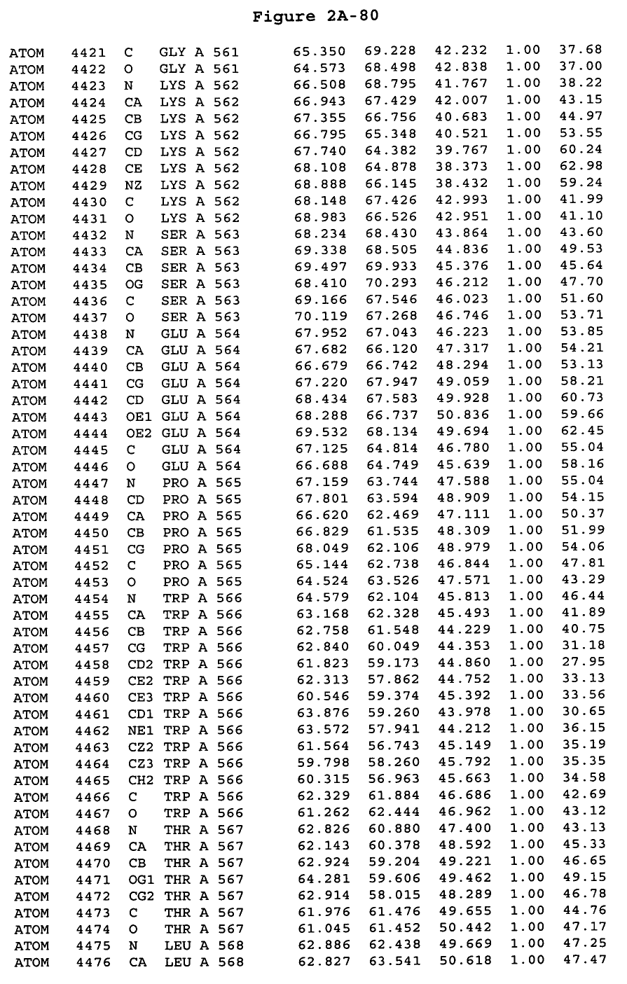

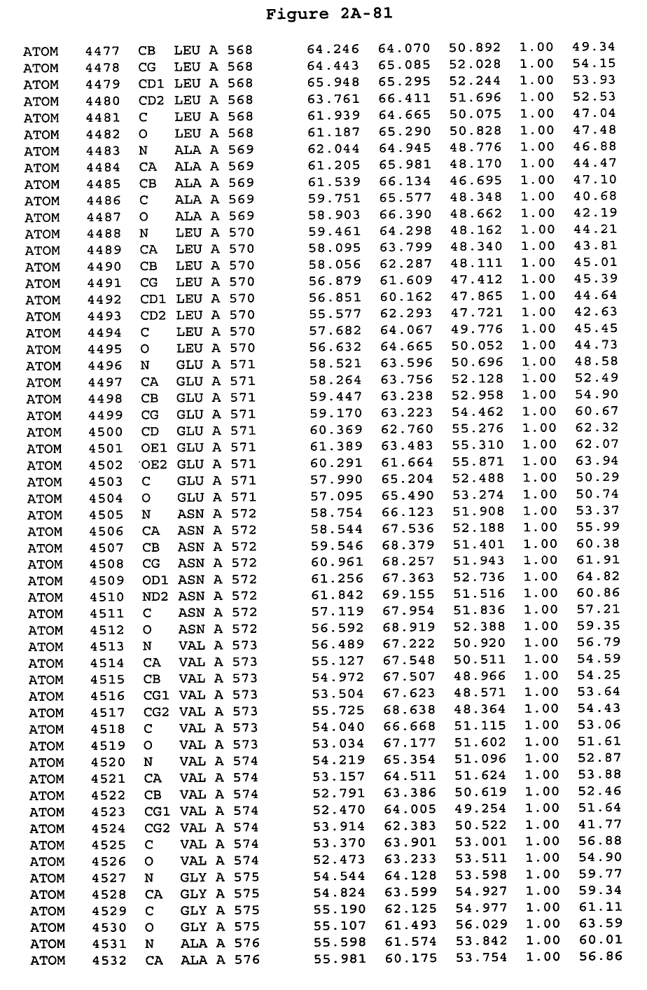

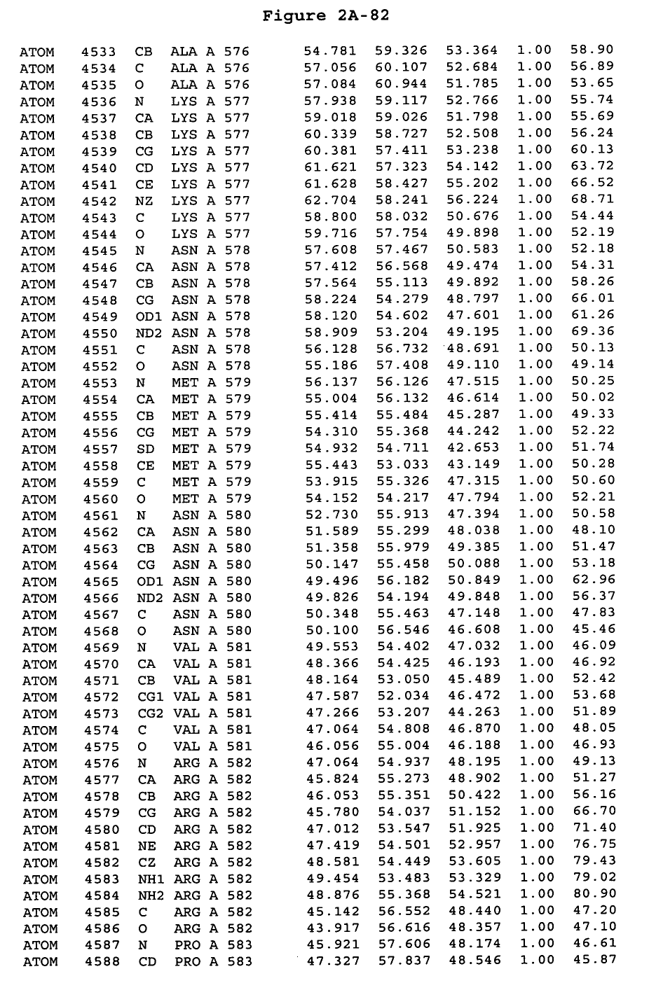

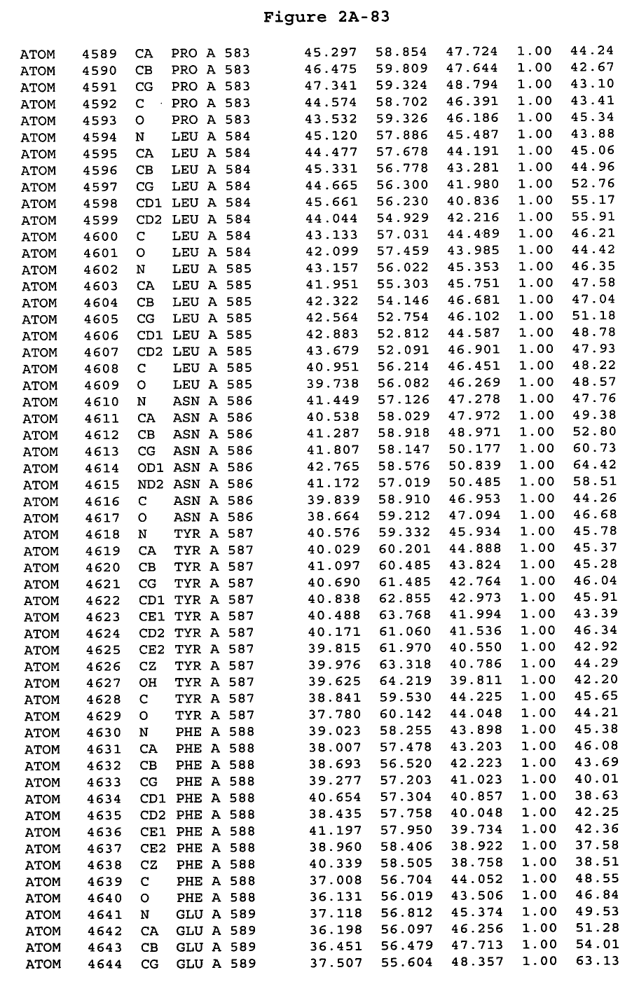

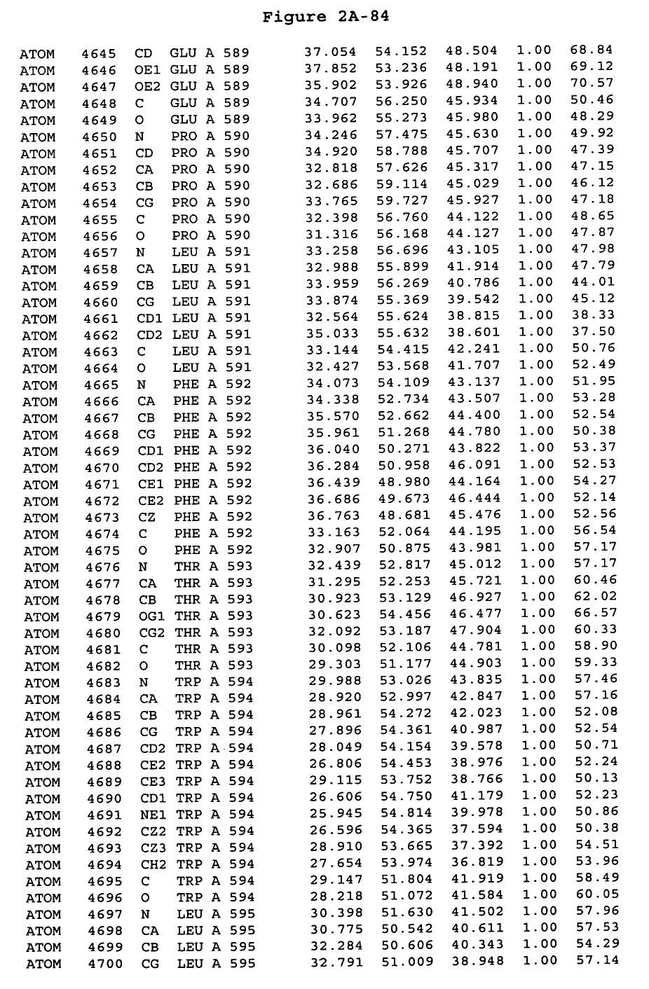

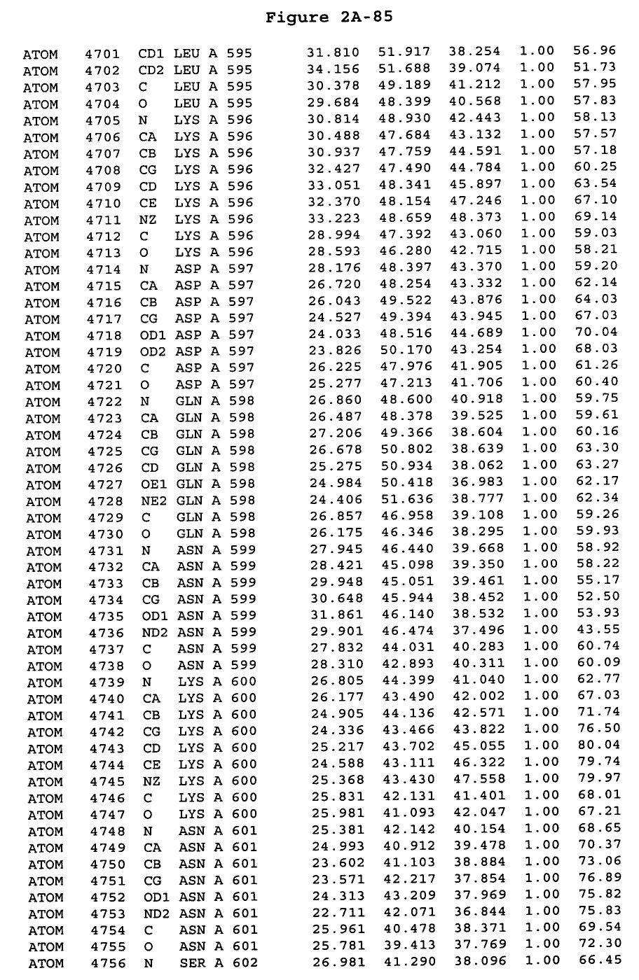

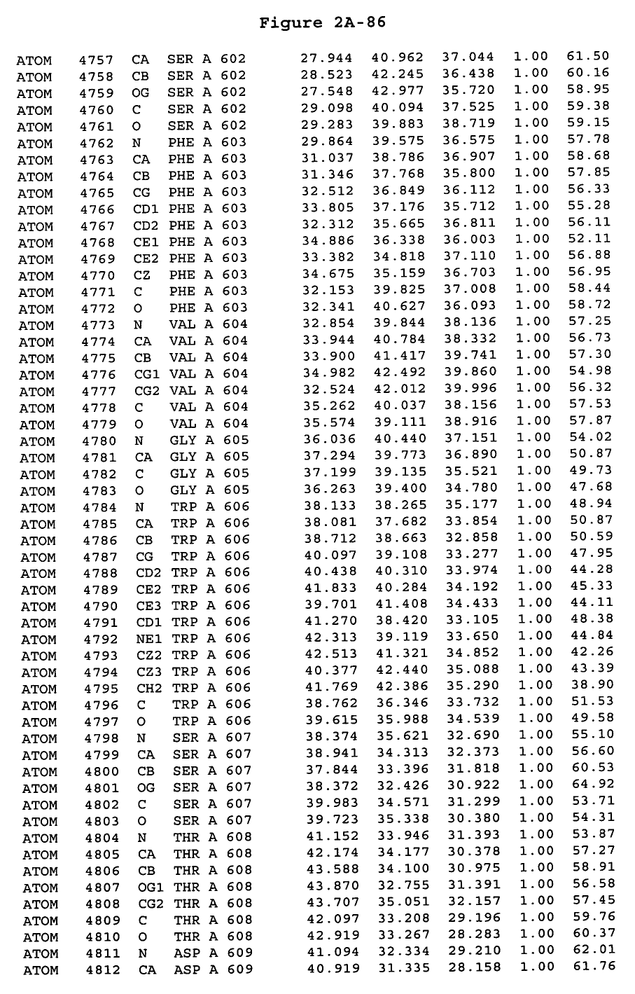

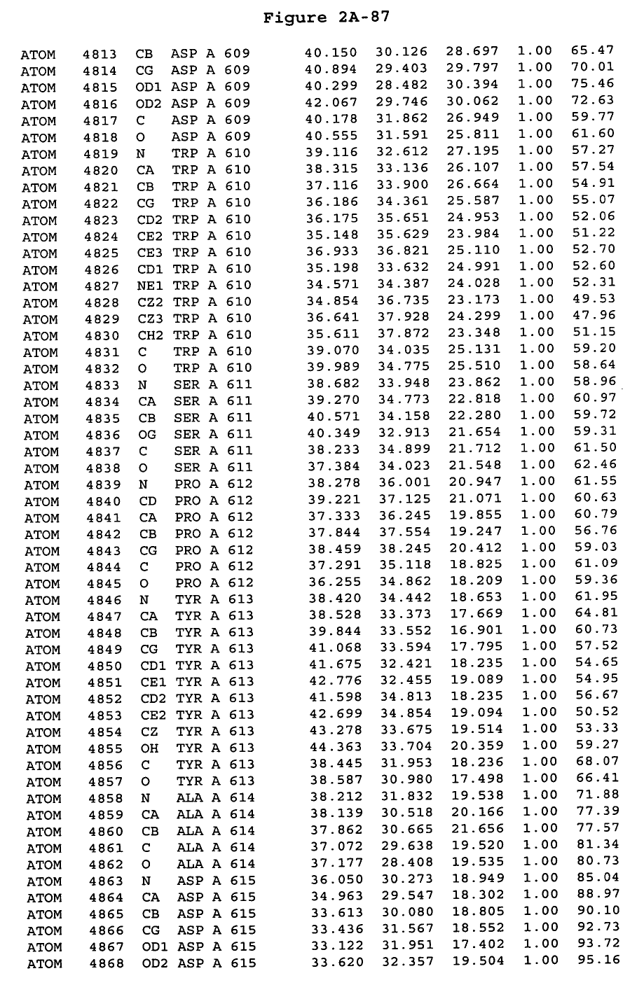

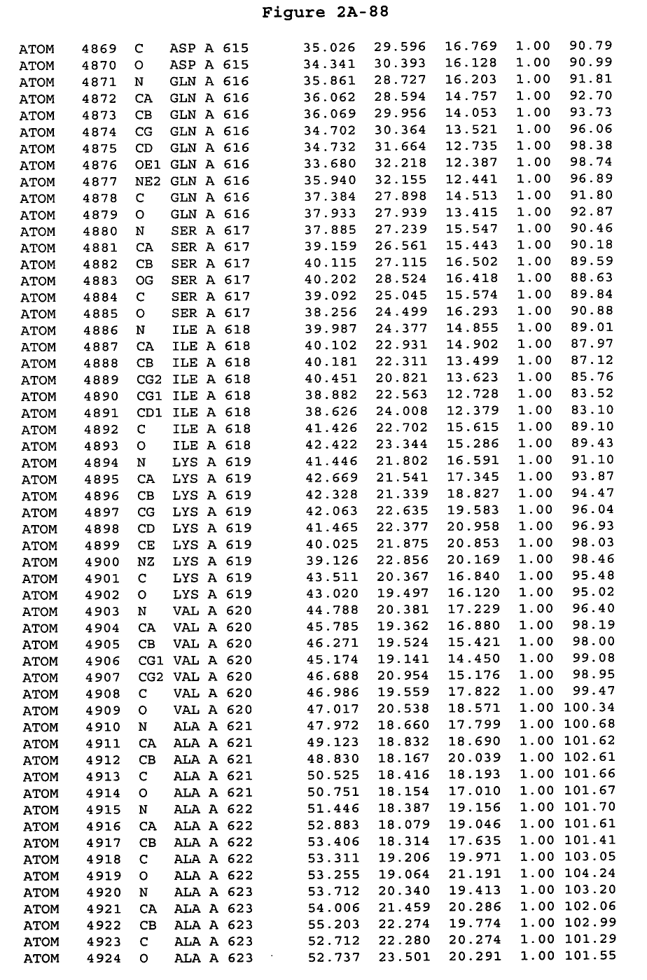

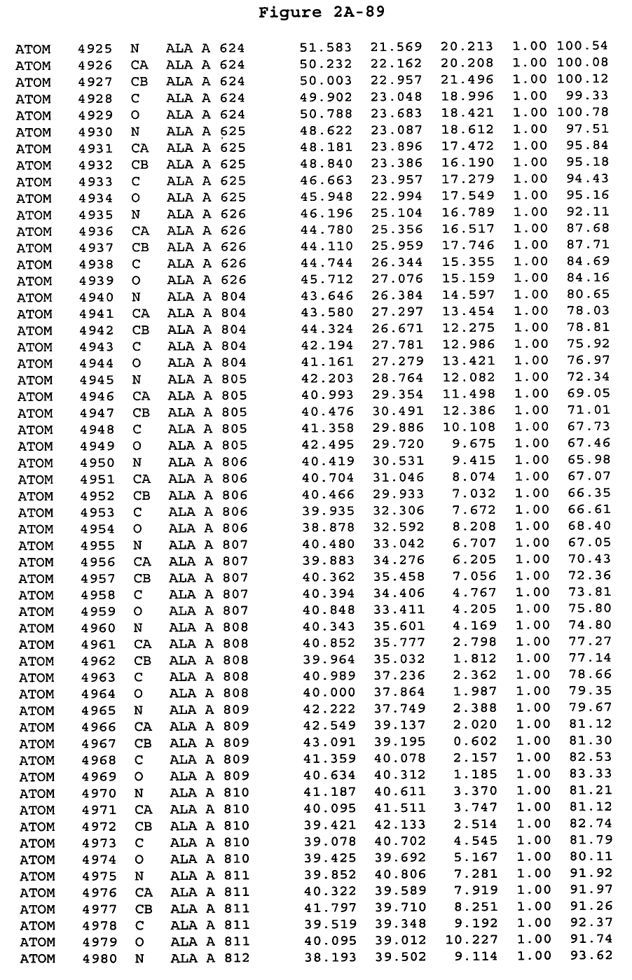

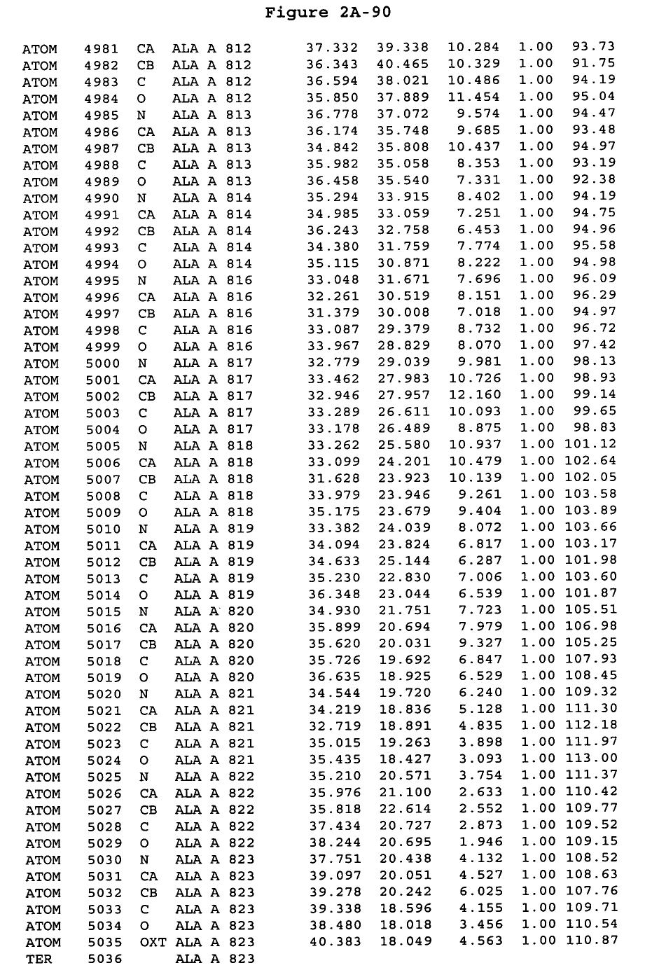

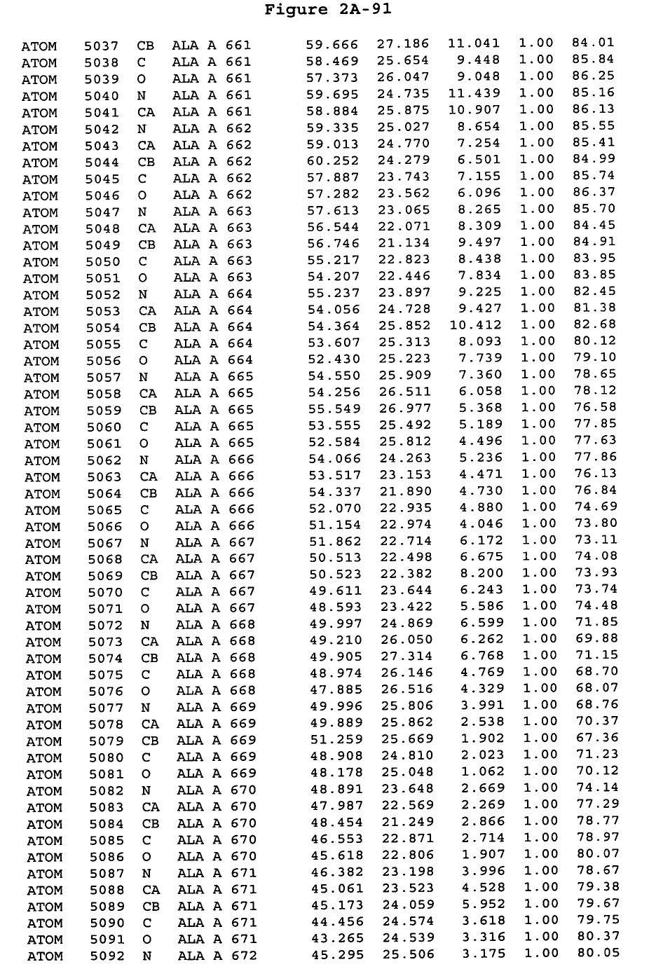

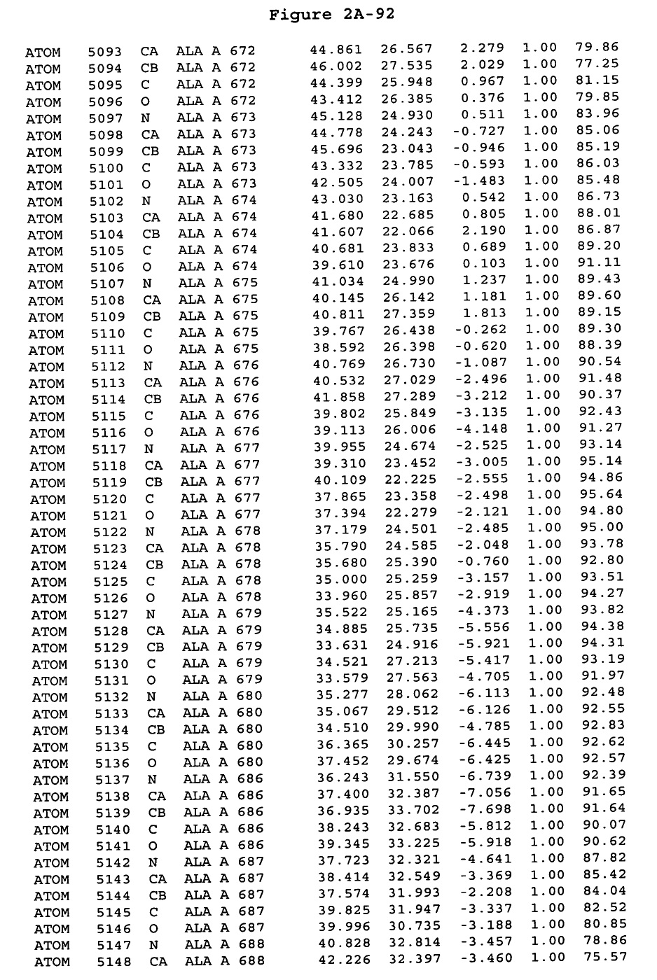

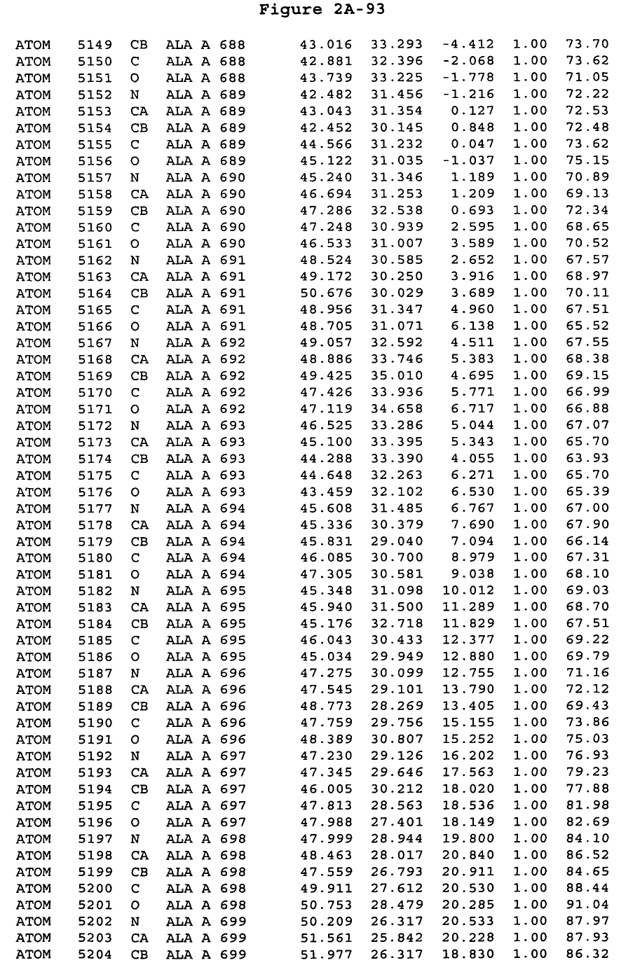

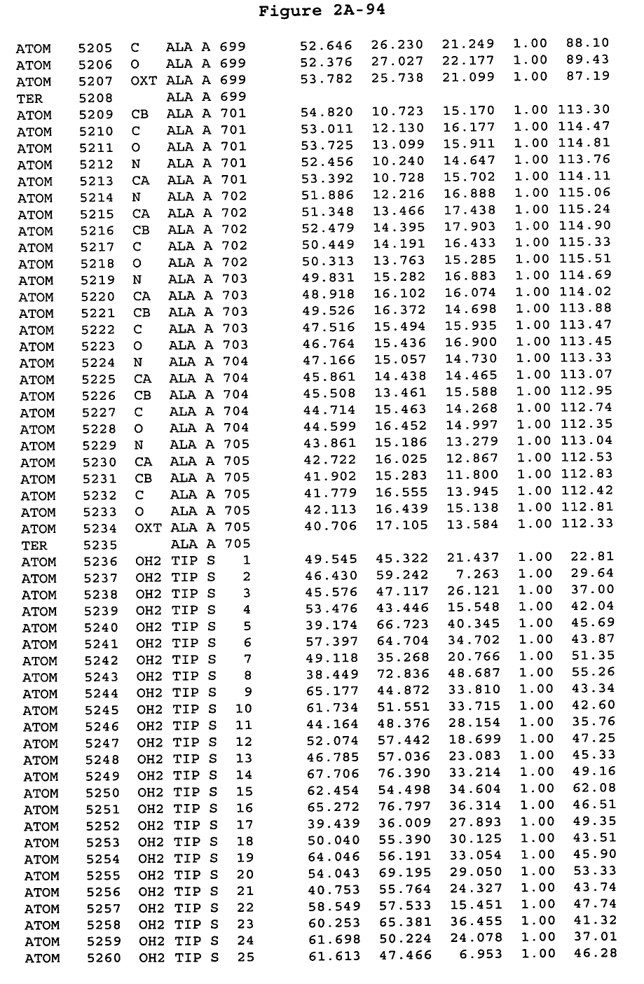

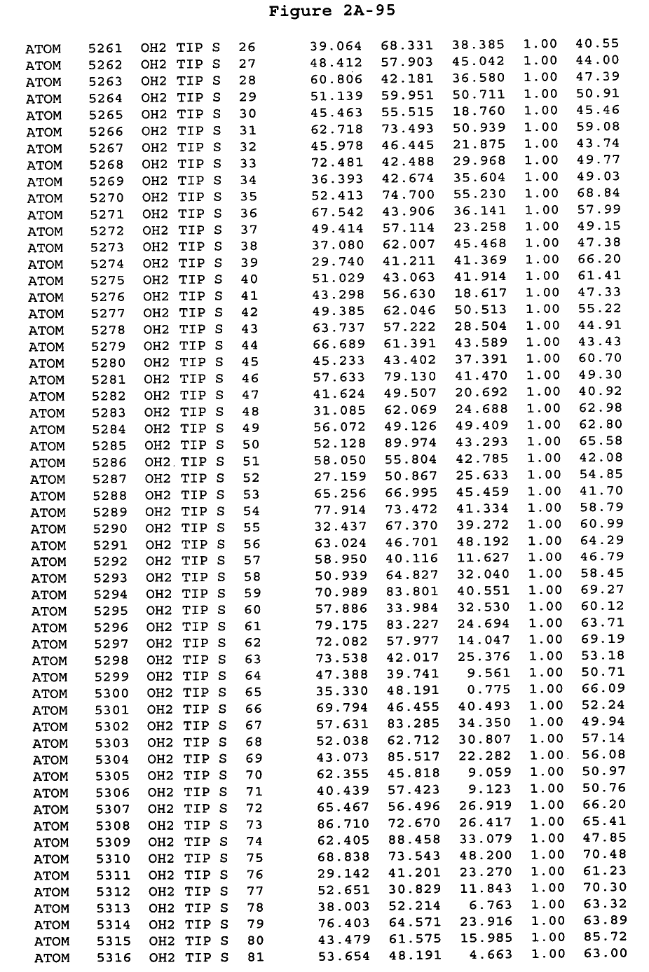

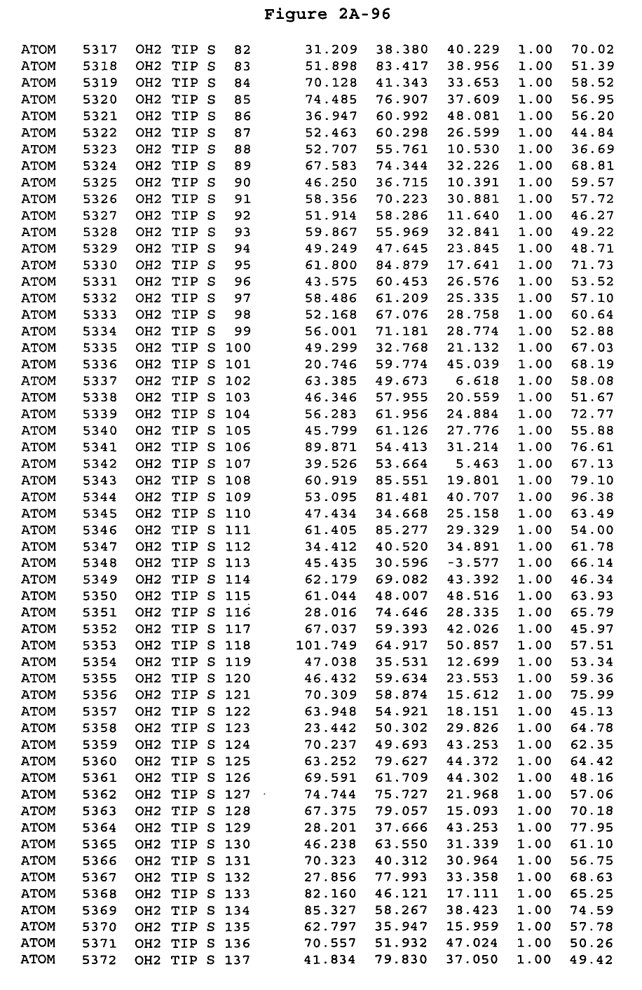

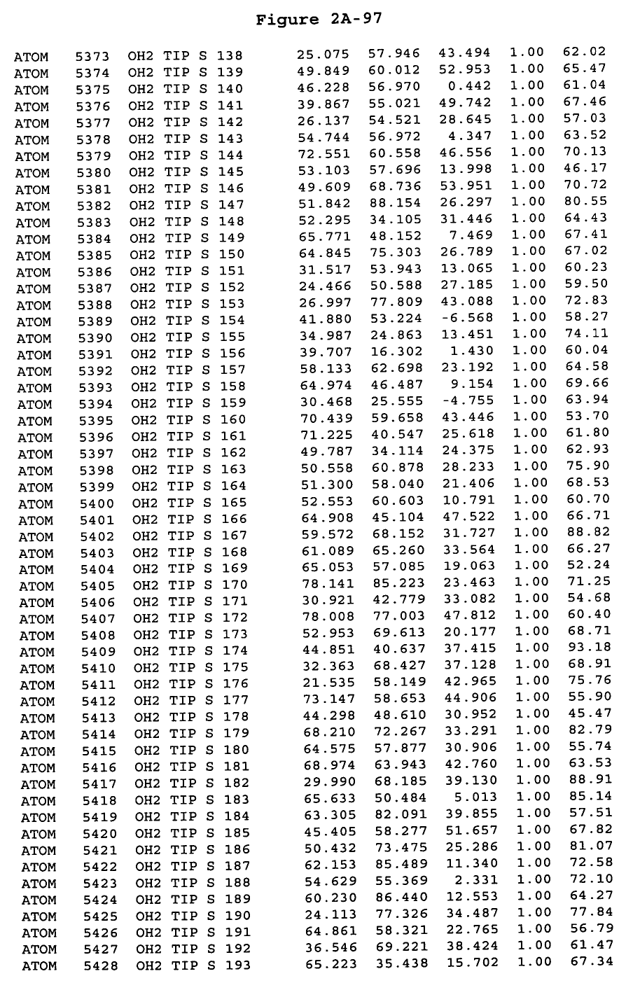

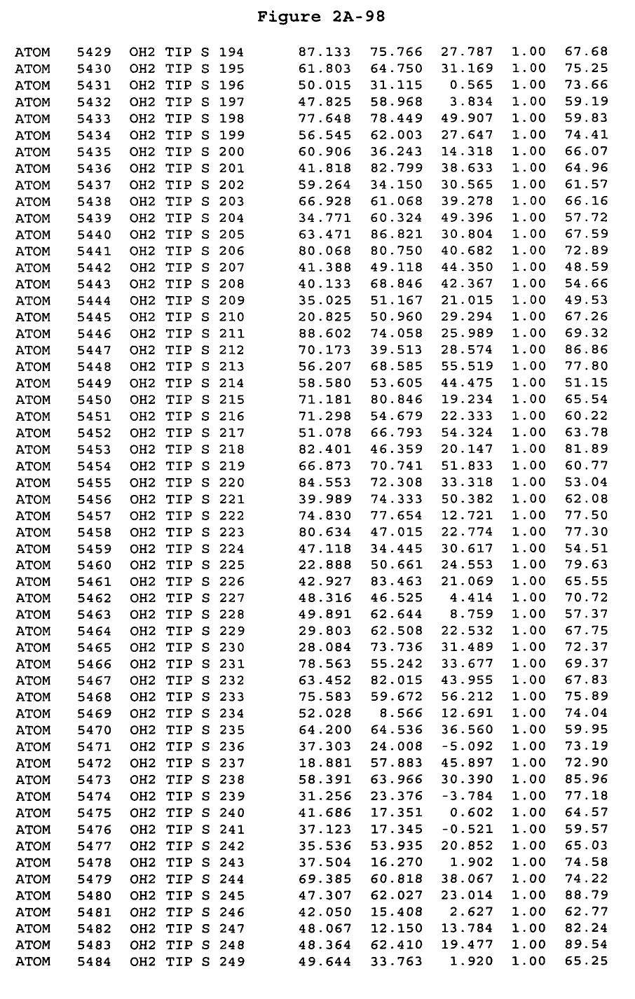

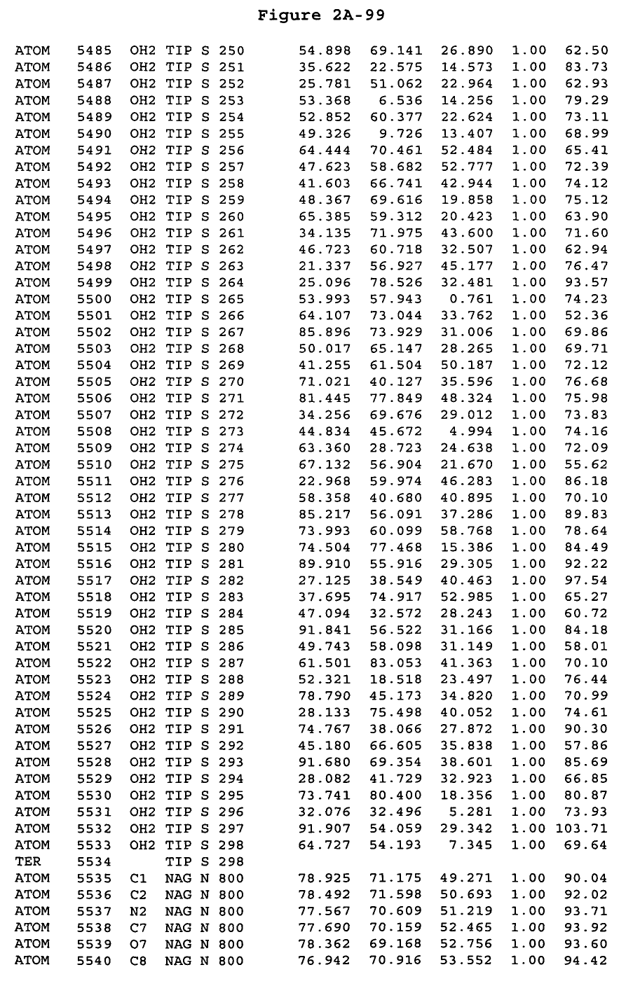

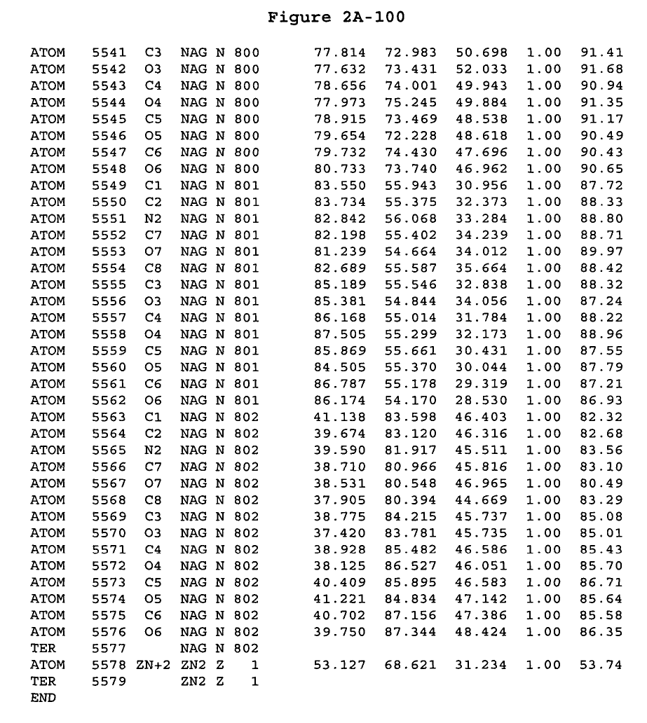

In one embodiment, the present invention provides a molecule or molecular complex comprising all or part of an ACE2 binding pocket defined by structure coordinates of a set of amino acid residues that correspond to human ACE2 amino acid residues Arg 273, Phe 274, His 374, Glu 375, His 401, Glu 402, Glu 406, His 505, Tyr 510, Arg 514, Tyr 515 and Arg 518 according to FIG. 1A or 2A, wherein the root mean square deviation of the backbone atoms between said amino acid residues of said molecule or molecular complex and said ACE2 amino acid residues is not more than about 3.0 Å. In one embodiment, the RMSD is not greater than about 2.0 Å. In one embodiment, the RMSD is not greater than about 1.0 Å. In one embodiment, the RMSD is not greater than about 0.8 Å. In one embodiment, the RMSD is not greater than about 0.5 Å. In one embodiment, the RMSD is not greater than about 0.3 Å. In one embodiment, the RMSD is not greater than about 0.2 Å.

In one embodiment, the present invention provides a molecule or molecular complex comprising all or part of an ACE2 binding pocket defined by structure coordinates of a set of amino acid residues that correspond to human ACE2 amino acid residues Arg 273, Phe 274, Glu 406, His 505, Tyr 510, Tyr 515 and Arg 518 according to FIG. 1A or 2A, wherein the root mean square deviation of the backbone atoms between said amino acid residues of said molecule or molecular complex and said ACE2 amino acid residues is not more than about 3.0 Å. In one embodiment, the RMSD is not greater than about 2.0 Å. In one embodiment, the RMSD is not greater than about 1.0 Å. In one embodiment, the RMSD is not greater than about 0.8 Å. In one embodiment, the RMSD is not greater than about 0.5 Å. In one embodiment, the RMSD is not greater than about 0.3 Å. In one embodiment, the RMSD is not greater than about 0.2 Å.

In one embodiment, the present invention provides a molecule or molecular complex comprising all or part of an ACE2 binding pocket defined by structure coordinates of a set of amino acid residues that correspond to human ACE2 amino acid residues Pro 346, Thr 347, Glu 402, Phe 504, Tyr 510, Arg 514 and Tyr 515 according to FIG. 1A or 2A, wherein the root mean square deviation of the backbone atoms between said amino acid residues of said molecule or molecular complex and said ACE2 amino acid residues is not more than about 3.0 Å. In one embodiment, the RMSD is not greater than about 2.0 Å. In one embodiment, the RMSD is not greater than about 1.0 Å. In one embodiment, the RMSD is not greater than about 0.8 Å. In one embodiment, the RMSD is not greater than about 0.5 Å. In one embodiment, the RMSD is not greater than about 0.3 Å. In one embodiment, the RMSD is not greater than about 0.2 Å.

In one embodiment, the present invention provides a molecule or molecular complex comprising all or part of an ACE2 binding pocket defined by structure coordinates of a set of amino acid residues that correspond to human ACE2 amino acid residues Pro 346, Thr 347 and Tyr 510 according to FIG. 1A or 2A, wherein the root mean square deviation of the backbone atoms between said amino acid residues of said molecule or molecular complex and said ACE2 amino acid residues is not more than about 3.0 Å. In one embodiment, the RMSD is not greater than about 2.0 Å. In one embodiment, the RMSD is not greater than about 1.0 Å. In one embodiment, the RMSD is not greater than about 0.8 Å. In one embodiment, the RMSD is not greater than about 0.5 Å. In one embodiment, the RMSD is not greater than about 0.3 Å. In one embodiment, the RMSD is not greater than about 0.2 Å.

In one embodiment, the present invention provides a molecule or molecular complex comprising all or part of an ACE2 binding pocket defined by structure coordinates of a set of amino acid residues that correspond to human ACE2 amino acid residues His 379, Asp 382, Tyr 385, Asn 394, His 401, Glu 402, Arg 514 according to FIG. 1A or 2A, wherein the root mean square deviation of the backbone atoms between said amino acid residues of said molecule or molecular complex and said ACE2 amino acid residues is not more than about 3.0 Å. In one embodiment, the RMSD is not greater than about 2.0 Å. In one embodiment, the RMSD is not greater than about 1.0 Å. In one embodiment, the RMSD is not greater than about 0.8 Å. In one embodiment, the RMSD is not greater than about 0.5 Å. In one embodiment, the RMSD is not greater than about 0.3 Å. In one embodiment, the RMSD is not greater than about 0.2 Å.

In one embodiment, the present invention provides a molecule or molecular complex comprising all or part of an ACE2 binding pocket defined by structure coordinates of a set of amino acid residues that correspond to human ACE2 amino acid residues Arg 273, Phe 274, His 345, Pro 346, Thr 371, His 374, Glu 406, Ser 409 and Arg 518 according to FIG. 1A or 2A, wherein the root mean square deviation of the backbone atoms between said amino acid residues of said molecule or molecular complex and said ACE2 amino acid residues is not more than about 3.0 Å. In one embodiment, the RMSD is not greater than about 2.0 Å. In one embodiment, the RMSD is not greater than about 1.0 Å. In one embodiment, the RMSD is not greater than about 0.8 Å. In one embodiment, the RMSD is not greater than about 0.5 Å. In one embodiment, the RMSD is not greater than about 0.3 Å. In one embodiment, the RMSD is not greater than about 0.2 Å.

In one embodiment, the present invention provides a molecule or molecular complex comprising all or part of an ACE2 binding pocket defined by structure coordinates of a set of amino acid residues that correspond to human ACE2 amino acid residues Arg 273, Glu 406 and Arg 518 according to FIG. 1A or 2A, wherein the root mean square deviation of the backbone atoms between said amino acid residues of said molecule or molecular complex and said ACE2 amino acid residues is not more than about 3.0 Å. In one embodiment, the RMSD is not greater than about 2.0 Å. In one embodiment, the RMSD is not greater than about 1.0 Å. In one embodiment, the RMSD is not greater than about 0.8 Å. In one embodiment, the RMSD is not greater than about 0.5 Å. In one embodiment, the RMSD is not greater than about 0.3 Å. In one embodiment, the RMSD is not greater than about 0.2 Å.

In one embodiment, the present invention provides a molecule or molecular complex comprising all or part of an ACE2 binding pocket defined by structure coordinates of a set of amino acid residues that correspond to human ACE2 amino acid residues Arg 273, His 505 and Tyr 515 according to FIG. 1A or 2A, wherein the root mean square deviation of the backbone atoms between said amino acid residues of said molecule or molecular complex and said ACE2 amino acid residues is not more than about 3.0 Å. In one embodiment, the RMSD is not greater than about 2.0 Å. In one embodiment, the RMSD is not greater than about 1.0 Å. In one embodiment, the RMSD is not greater than about 0.8 Å. In one embodiment, the RMSD is not greater than about 0.5 Å. In one embodiment, the RMSD is not greater than about 0.3 Å. In one embodiment, the RMSD is not greater than about 0.2 Å.

In one embodiment, the present invention provides a molecule or molecular complex comprising all or part of an ACE2 binding pocket defined by structure coordinates of a set of amino acid residues that correspond to human ACE2 amino acid residues Phe 40, Ser 44, Thr 347, Trp 349, Asp 382, Tyr 385, Asn 394 according to FIG. 1A or 2A, wherein the root mean square deviation of the backbone atoms between said amino acid residues of said molecule or molecular complex and said ACE2 amino acid residues is not more than about 3.0 Å. In one embodiment, the RMSD is not greater than about 2.0 Å. In one embodiment, the RMSD is not greater than about 1.0 Å. In one embodiment, the RMSD is not greater than about 0.8 Å. In one embodiment, the RMSD is not greater than about 0.5 Å. In one embodiment, the RMSD is not greater than about 0.3 Å. In one embodiment, the RMSD is not greater than about 0.2 Å.

In another embodiment, the present invention provides a molecule or molecular complex, preferably a crystalline molecule or molecular complex, comprising all or part of an ACE2 binding pocket defined by structure coordinates of at least 3, 5, 7 or 10 of a set of amino acid residues that correspond to human ACE2 amino acid residues selected from the group consisting of Phe 40, Ser 44, Trp 69, Ser 70, Leu 73, Lys 74, Ser 77, Thr 78, Leu 85, Leu 91, Thr 92, Lys 94, Leu 95, Gln 96, Gln 98, Ala 99, Leu 100, Gln 101, Gln 102, Asn 103, Gly 104, Ser 106, Asn 194, His 195, Tyr 196, Tyr 199, Tyr 202, Trp 203, Arg 204, Gly 205, Asp 206, Tyr 207, Glu 208, Val 209, Asn 210, Val 212, Arg 219, Arg 273, Phe 274, Thr 276, Tyr 279, Pro 289, Asn 290, Ile 291, Cys 344, His 345, Pro 346, Thr 347, Ala 348, Trp 349, Asp 350, Leu 351, Gly 352, Cys 361, Met 366, Asp 367, Asp 368, Leu 370, Thr 371, His 374, Glu 375, His 378, Asp 382, Tyr 385, Phe 390, Leu 391, Leu 392, Arg 393, Asn 394, Gly 395, Ala 396, Asn 397, Glu 398, Gly 399, Phe 400, His 401, Glu 402, Ala 403, Glu 406, Ser 409, Leu 410, Ala 413, Thr 414, Pro 415, Leu 418, Phe 428, Glu 430, Asp 431, Asn 432, Thr 434, Glu 435, Asn 437, Phe 438, Lys 441, Gln 442, Thr 445, Ile 446, Thr 449, Leu 450, Arg 460, Phe 504, His 505, Ser 507, Asn 508, Asp 509, Tyr 510, Ser 511, Arg 514, Tyr 515, Arg 518, Thr 519, Gln 522, His 540, Lys 541, Lys 562, Ser 563, Glu 564, Pro 565, Trp 566 and Tyr 587 according to FIG. 1A or 2A, wherein the RMSD of the backbone atoms between said amino acid residues and said ACE2 amino acid residues is not greater than about 3.0 Å. In one embodiment, the RMSD is not greater than about 2.0 Å. In one embodiment, the RMSD is not greater than about 1.0 Å. In one embodiment, the RMSD is not greater than about 0.8 Å. In one embodiment, the RMSD is not greater than about 0.5 Å. In one embodiment, the RMSD is not greater than about 0.3 Å. In one embodiment, the RMSD is not greater than about 0.2 Å.

In one embodiment, the present invention provides a molecule or molecular complex comprising all or part of an ACE2 binding pocket defined by structure coordinates of a set of amino acid residues that correspond to human ACE2 amino acid residues N149, D269, R273, F274, H345, P346, A348, D367, T371, H374, E375, H378, E398, E402, R481, L503, F504, H505, Y510, S511, F512, Y515 and E564 according to FIG. 3A or 3B, wherein the root mean square deviation of the backbone atoms between said amino acid residues of said molecule or molecular complex and said ACE2 amino acid residues is not more than about 3.0 Å. In one embodiment, the RMSD is not greater than about 2.0 Å. In one embodiment, the RMSD is not greater than about 1.0 Å. In one embodiment, the RMSD is not greater than about 0.8 Å. In one embodiment, the RMSD is not greater than about 0.5 Å. In one embodiment, the RMSD is not greater than about 0.3 Å. In one embodiment, the RMSD is not greater than about 0.2 Å.

In one embodiment, the present invention provides a molecule or molecular complex comprising all or part of an ACE2 binding pocket defined by structure coordinates of a set of amino acid residues that correspond to human ACE2 amino acid residues N149, D269, R273, F274, H345, P346, A348, D367, T371, H374, E375, H378, E398, E402, R481, L503, F504, H505, Y510, S511, F512 and Y515 according to FIG. 3A or 3B, wherein the root mean square deviation of the backbone atoms between said amino acid residues of said molecule or molecular complex and said ACE2 amino acid residues is not more than about 3.0 Å. In one embodiment, the RMSD is not greater than about 2.0 Å. In one embodiment, the RMSD is not greater than about 1.0 Å. In one embodiment, the RMSD is not greater than about 0.8 Å. In one embodiment, the RMSD is not greater than about 0.5 Å. In one embodiment, the RMSD is not greater than about 0.3 Å. In one embodiment, the RMSD is not greater than about 0.2 Å.

In one embodiment, the present invention provides a molecule or molecular complex comprising all or part of an ACE2 binding pocket defined by structure coordinates of a set of amino acid residues that correspond to human ACE2 amino acid residues N149, D269, R273, F274, H345, P346, A348, D367, T371, H374, E375, H378, E402, F504, H505, Y510, F512, and Y515 according to FIG. 3A or 3B, wherein the root mean square deviation of the backbone atoms between said amino acid residues of said molecule or molecular complex and said ACE2 amino acid residues is not more than about 3.0 Å. In one embodiment, the RMSD is not greater than about 2.0 Å. In one embodiment, the RMSD is not greater than about 1.0 Å. In one embodiment, the RMSD is not greater than about 0.8 Å. In one embodiment, the RMSD is not greater than about 0.5 Å. In one embodiment, the RMSD is not greater than about 0.3 Å. In one embodiment, the RMSD is not greater than about 0.2 Å.

In one embodiment, the present invention provides a molecule or molecular complex comprising all or part of an ACE2 binding pocket defined by structure coordinates of a set of amino acid residues that correspond to human ACE2 amino acid residues N149, D269, R273, F274, P346, T371, Y510, and F512 according to FIG. 3A or 3B, wherein the root mean square deviation of the backbone atoms between said amino acid residues of said molecule or molecular complex and said ACE2 amino acid residues is not more than about 3.0 Å. In one embodiment, the RMSD is not greater than about 2.0 Å. In one embodiment, the RMSD is not greater than about 1.0 Å. In one embodiment, the RMSD is not greater than about 0.8 Å. In one embodiment, the RMSD is not greater than about 0.5 Å. In one embodiment, the RMSD is not greater than about 0.3 Å. In one embodiment, the RMSD is not greater than about 0.2 Å.

In one embodiment, the present invention provides a molecule or molecular complex comprising all or part of an ACE2 binding pocket defined by structure coordinates of a set of amino acid residues that correspond to human ACE2 amino acid residues R273, F274, P346, and T371 according to FIG. 3A or 3B, wherein the root mean square deviation of the backbone atoms between said amino acid residues of said molecule or molecular complex and said ACE2 amino acid residues is not more than about 3.0 Å. In one embodiment, the RMSD is not greater than about 2.0 Å. In one embodiment, the RMSD is not greater than about 1.0 Å. In one embodiment, the RMSD is not greater than about 0.8 Å. In one embodiment, the RMSD is not greater than about 0.5 Å. In one embodiment, the RMSD is not greater than about 0.3 Å. In one embodiment, the RMSD is not greater than about 0.2 Å.

In one embodiment, the present invention provides a molecule or molecular complex comprising all or part of an ACE2 binding pocket defined by structure coordinates of a set of amino acid residues that correspond to human ACE2 amino acid residues R273, F274, H345, P346, T371, H374, E375, H378, E402, H505, Y510 and Y515 according to FIG. 3A or 3B, wherein the root mean square deviation of the backbone atoms between said amino acid residues of said molecule or molecular complex and said ACE2 amino acid residues is not more than about 3.0 Å. In one embodiment, the RMSD is not greater than about 2.0 Å. In one embodiment, the RMSD is not greater than about 1.0 Å. In one embodiment, the RMSD is not greater than about 0.8 Å. In one embodiment, the RMSD is not greater than about 0.5 Å. In one embodiment, the RMSD is not greater than about 0.3 Å. In one embodiment, the RMSD is not greater than about 0.2 Å.

In one embodiment, the present invention provides a molecule or molecular complex comprising all or part of an ACE2 binding pocket defined by structure coordinates of a set of amino acid residues that correspond to human ACE2 amino acid residues R273, F274, H345, P346, T371, H374, E375, H378, E402, H505, and Y515 according to FIG. 3A or 3B, wherein the root mean square deviation of the backbone atoms between said amino acid residues of said molecule or molecular complex and said ACE2 amino acid residues is not more than about 3.0 Å. In one embodiment, the RMSD is not greater than about 2.0 Å. In one embodiment, the RMSD is not greater than about 1.0 Å. In one embodiment, the RMSD is not greater than about 0.8 Å. In one embodiment, the RMSD is not greater than about 0.5 Å. In one embodiment, the RMSD is not greater than about 0.3 Å. In one embodiment, the RMSD is not greater than about 0.2 Å.

In one embodiment, the present invention provides a molecule or molecular complex comprising all or part of an ACE2 binding pocket defined by structure coordinates of a set of amino acid residues that correspond to human ACE2 amino acid residues R273, F274, H345, P346, D367, T371, H374, E375, H378, E402, H505, Y510, R514 and Y515 according to FIG. 3A or 3B, wherein the root mean square deviation of the backbone atoms between said amino acid residues of said molecule or molecular complex and said ACE2 amino acid residues is not more than about 3.0 Å. In one embodiment, the RMSD is not greater than about 2.0 Å. In one embodiment, the RMSD is not greater than about 1.0 Å. In one embodiment, the RMSD is not greater than about 0.8 Å. In one embodiment, the RMSD is not greater than about 0.5 Å. In one embodiment, the RMSD is not greater than about 0.3 Å. In one embodiment, the RMSD is not greater than about 0.2 Å.

In one embodiment, the present invention provides a molecule or molecular complex comprising all or part of an ACE2 binding pocket defined by structure coordinates of a set of amino acid residues that correspond to human ACE2 amino acid residues N149, D269, R273, F274, H345, P346, A348, D367, T371, H374, E375, H378, E402, F504, H505, Y510, F512, R514, and Y515 according to FIG. 3A or 3B, wherein the root mean square deviation of the backbone atoms between said amino acid residues of said molecule or molecular complex and said ACE2 amino acid residues is not more than about 3.0 Å. In one embodiment, the RMSD is not greater than about 2.0 Å. In one embodiment, the RMSD is not greater than about 1.0 Å. In one embodiment, the RMSD is not greater than about 0.8 Å. In one embodiment, the RMSD is not greater than about 0.5 Å. In one embodiment, the RMSD is not greater than about 0.3 Å. In one embodiment, the RMSD is not greater than about 0.2 Å.

In one embodiment, the present invention provides a molecule or molecular complex comprising all or part of an ACE2 binding pocket defined by structure coordinates of a set of amino acid residues that correspond to human ACE2 amino acid residues N149, D269, R273, F274, P346, T371, E398, R481, L503, Y510, S511, F512, and E564 according to FIG. 3A or 3B, wherein the root mean square deviation of the backbone atoms between said amino acid residues of said molecule or molecular complex and said ACE2 amino acid residues is not more than about 3.0 Å. In one embodiment, the RMSD is not greater than about 2.0 Å. In one embodiment, the RMSD is not greater than about 1.0 Å. In one embodiment, the RMSD is not greater than about 0.8 Å. In one embodiment, the RMSD is not greater than about 0.5 Å. In one embodiment, the RMSD is not greater than about 0.3 Å. In one embodiment, the RMSD is not greater than about 0.2 Å.

In one embodiment, the present invention provides a molecule or molecular complex comprising all or part of an ACE2 binding pocket defined by structure coordinates of a set of amino acid residues that correspond to human ACE2 amino acid residues N149, D269, R273, F274, H345, P346, A348, D367, T371, H374, E375, H378, E398, E402, R481, L503, F504, H505, Y510, S511, F512, R514, Y515 and E564 according to FIG. 3A or 3B, wherein the root mean square deviation of the backbone atoms between said amino acid residues of said molecule or molecular complex and said ACE2 amino acid residues is not more than about 3.0 Å. In one embodiment, the RMSD is not greater than about 2.0 Å. In one embodiment, the RMSD is not greater than about 1.0 Å. In one embodiment, the RMSD is not greater than about 0.8 Å. In one embodiment, the RMSD is not greater than about 0.5 Å. In one embodiment, the RMSD is not greater than about 0.3 Å. In one embodiment, the RMSD is not greater than about 0.2 Å.

Another embodiment of this invention provides a molecule or molecular complex comprising a protein defined by structure coordinates of a set of amino acid residues which correspond to human ACE2 amino acid residues according to FIG. 1A, 2A, 3A or 3B, wherein the root mean square deviation between said set of amino acid residues of said molecule or molecular complex and said ACE2 amino acid residues is not more than about 3 Å. In one embodiment, the RMSD is not greater than about 2 Å. In one embodiment, the RMSD is not greater than about 1.7 Å. In one embodiment, the RMSD is not greater than about 1.5 Å. In one embodiment, the RMSD is not greater than about 1.0 Å. In one embodiment, the RMSD is not greater than about 0.5 Å. Alanines were built in the molecular model of FIGS. 1A and 2A due to weak electron density. For the purpose of this invention, human ACE2 amino acid residues refer to the amino acid identities shown in SEQ ID NO:4.

In one embodiment, the above molecules or molecular complexes are ACE2 proteins or ACE2 homologues. In another embodiment, the above molecules or molecular complexes are in crystalline form. An ACE2 protein may be human ACE2. Homologues of human ACE2 can be ACE2 from another species, such as a mouse, a rat or a non-human primate.

Computer Systems

According to another embodiment, this invention provided a machine-readable data storage medium, comprising a data storage material encoded with machine-readable data, wherein said data defines the above-mentioned molecules or molecular complexes. In one embodiment, the data defines the above-mentioned binding pockets by comprising the structure coordinates of said amino acid residues according to FIGS. 1A, 2A, 3A or 3B. To use the structure coordinates generated for ACE2, homologues thereof, or one of its binding pockets, it is at times necessary to convert them into a three-dimensional shape. This is achieved through the use of commercially or publicly available software that is capable of generating a three-dimensional structure of molecules or portions thereof from a set of structure coordinates. The three-dimensional structure may be displayed as a graphical representation on a machine, such as a computer.

Therefore, according to another embodiment, this invention provides a machine-readable data storage medium comprising a data storage material encoded with machine readable data. In one embodiment, a machine programmed with instructions for using said data is capable of generating a three-dimensional structure of any of the crystalline molecule or molecular complexes, or binding pockets thereof, that are described herein.

This invention also provides a computer comprising:

(a) a machine-readable data storage medium comprising a data storage material encoded with machine-readable data, wherein said data defines any one of the above molecules or molecular complexes;

(b) a working memory for storing instructions for processing said machine-readable data;

(c) a central processing unit (CPU) coupled to said working memory and to said machine-readable data storage medium for processing said machine readable data and means for generating three-dimensional structural information of said molecule or molecular complex; and

(d) output hardware coupled to said central processing unit for outputting three-dimensional structural information of said molecule or molecular complex, or information produced using said three-dimensional structural information of said molecule or molecular complex.

In one embodiment, the data defines the binding pocket or protein of the molecule or molecular complex.

Three-dimensional data generation may be provided by an instruction or set of instructions such as a computer program or commands for generating a three-dimensional structure or graphical representation from structure coordinates, or by subtracting distances between atoms, calculating chemical energies for an ACE2 molecule or molecular complex or homologues thereof, or calculating or minimizing energies for an association of an ACE2 molecule or molecular complex or homologues thereof to a chemical entity. The graphical representation can be generated or displayed by commercially available software programs. Examples of software programs include but are not limited to QUANTA (Molecular Simulations, Inc., San Diego, Calif. (1998, 2000; Accelrys ©2001, 2002), O (Jones et al., Acta Crystallogr. A47, pp. 110-119 (1991)) and RIBBONS)Carson, J. Appl. Crystallogr., 24, pp. 9589-961 (1991)), which are incorporated herein by reference. Certain software programs may imbue this representation with physico-chemical attributes which are known from the chemical composition of the molecule, such as residue charge, hydrophobicity, torsional and rotational degrees of freedom for the residue or segment, etc. Examples of software programs for calculating chemical energies are described in the Rational Drug Design section.

In one embodiment, the computer is executing an instruction such as a computer program for three-dimensional data generation.

Information of said binding pocket or information produced by using said binding pocket can be outputted through display terminals, touchscreens, facsimile machines, modems, CD-ROMs, printers or disk drives. The information can be in graphical or alphanumeric form.

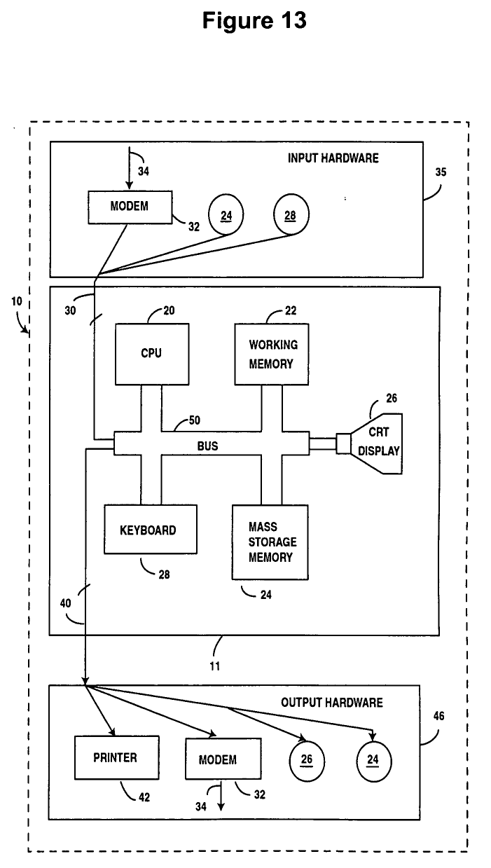

FIG. 13 demonstrates one version of these embodiments. System (10) includes a computer (11) comprising a central processing unit (“CPU”) (20), a working memory (22) which may be, e.g., RAM (random-access memory) or “core” memory, mass storage memory (24) (such as one or more disk drives, CD-ROM drives or DVD-ROM drives), one or more cathode-ray tube (“CRT”) display terminals (26), one or more keyboards (28), one or more input lines (30), and one or more output lines (40), all of which are, interconnected by a conventional bi-directional system bus (50).

Input hardware (35), coupled to computer (11) by input lines (30), may be implemented in a variety of ways. Machine-readable data of this invention may be inputted via the use of a modem or modems (32) connected by a telephone line or dedicated data line (34). Alternatively or additionally, the input hardware (35) may comprise CD-ROM or DVD-ROM drives or disk drives (24). In conjunction with display terminal (26), keyboard (28) may also be used as an input device.

Output hardware (46), coupled to computer (11) by output lines (40), may similarly be implemented by conventional devices. By way of example, output hardware (46) may include CRT display terminal (26) for displaying a graphical representation of a binding pocket of this invention using a program such as QUANTA (Molecular Simulations, Inc., San Diego, Calif. ©1998, 2000;Accelrys ©2001, 2002) as described herein. Output hardware may also include a printer (42), so that hard copy output may be produced, or a disk drive (24), to store system output for later use. Output hardware may also include a CD or DVD recorder, ZIP™ or JAZ™ drive, or other machine-readable data storage device.

In operation, CPU (20) coordinates the use of the various input and output devices (35), (46), coordinates data accesses from mass storage (24) and accesses to and from working memory (22), and determines the sequence of data processing steps. A number of programs may be used to process the machine-readable data of this invention. Such programs are discussed in reference to the computational methods of drug discovery as described herein. Specific references to components of the hardware system (10) are included as appropriate throughout the following description of the data storage medium.

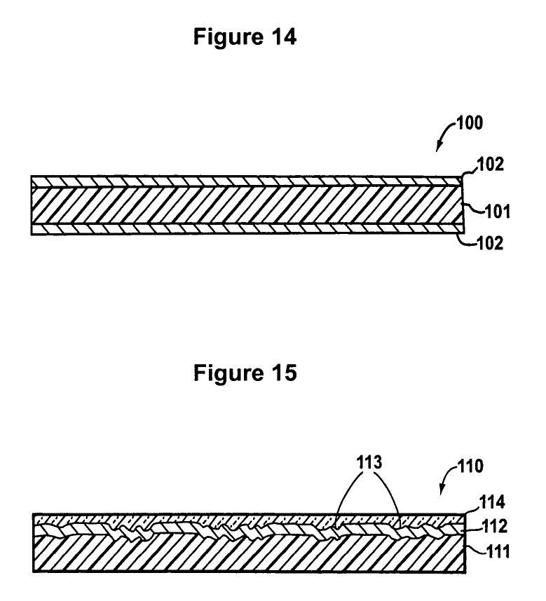

FIG. 14 shows a cross section of a magnetic data storage medium (100) which can be encoded with a machine-readable data that can be carried out by a system such as system (10) of FIG. 13. Medium (100) can be a conventional floppy diskette or hard disk, having a suitable substrate (101), which may be conventional, and a suitable coating (102), which may be conventional, on one or both sides, containing magnetic domains (not visible) whose polarity or orientation can be altered magnetically. Medium (100) may also have an opening (not shown) for receiving the spindle of a disk drive or other data storage device (24).

The magnetic domains of coating (102) of medium (100) are polarized or oriented so as to encode in manner which may be conventional, machine readable data such as that described herein, for execution by a system such as system (10) of FIG. 13.

FIG. 15 shows a cross section of an optically-readable data storage medium (110) which also can be encoded with such a machine-readable data, or set of instructions, which can be carried out by a system such as system (10) of FIG. 13. Medium (110) can be a conventional compact disk read only memory (CD-ROM) or a rewritable medium such as a magneto-optical disk which is optically readable and magneto-optically writable. Medium (100) preferably has a suitable substrate (111), which may be conventional, and a suitable coating (112), which may be conventional, usually of one side of substrate (111).

In the case of CD-ROM, as is well known, coating (112) is reflective and is impressed with a plurality of pits (113) to encode the machine-readable data. The arrangement of pits is read by reflecting laser light off the surface of coating (112). A protective coating (114), which preferably is substantially transparent, is provided on top of coating (112).

In the case of a magneto-optical disk, as is well known, coating (112) has no pits (113), but has a plurality of magnetic domains whose polarity or orientation can be changed magnetically when heated above a certain temperature, as by a laser (not shown). The orientation of the domains can be read by measuring the polarization of laser light reflected from coating (112). The arrangement of the domains encodes the data as described above.

In one embodiment, the structure coordinates of said molecules or molecular complexes are produced by homology modeling of at least a portion of the structure coordinates of FIGS. 1A, 2A, 3A or 3B. Homology modeling can be used to generate structural models of ACE2 homologues or other homologous proteins based on the known structure of ACE2. This can be achieved by performing one or more of the following steps: performing sequence alignment between the amino acid sequence of a molecule (possibly an unknown molecule) against the amino acid sequence of ACE2; identifying conserved and variable regions by sequence or structure; generating structure co-ordinates for structurally conserved residues of the unknown structure from those of ACE2; generating conformations for the structurally variable residues in the unknown structure; replacing the non-conserved residues of ACE2 with residues in the unknown structure; building side chain conformations; and refining and/or evaluating the unknown structure.

Software programs that are useful in homology modeling include XALIGN [Wishart, D. S. et al., Comput. Appl. Biosci., 10, pp. 687-88 (1994)] and CLUSTAL W Alignment Tool [Higgins D. G. et al., Methods Enzymol., 266, pp. 383-402 (1996)]. See also, U.S. Pat. No. 5,884,230. These references are incorporated herein by reference.

To perform the sequence alignment, programs such as the “bestfit” program available from the Genetics Computer Group (Waterman in Advances in Applied Mathematics_{—}2, 482 (1981), which is incorporated herein by reference) and CLUSTAL W Alignment Tool (Higgins et al., supra, which is incorporated by reference) can be used. To model the amino acid side chains of homologous molecules, the amino acid residues in ACE2 can be replaced, using a computer graphics program such as “0” (Jones et al, (1991) Acta Cryst. Sect. A, 47: 110-119), by those of the homologous protein, where they differ. The same orientation or a different orientation of the amino acid can be used. Insertions and deletions of amino acid residues may be necessary where gaps occur in the sequence alignment. However, certain portions of the active site of ACE2 and its homologues are highly conserved with essentially no insertions and deletions.

Homology modeling can be performed using, for example, the computer programs SWISS-MODEL available through Glaxo Wellcome Experimental Research in Geneva, Switzerland; WHATIF available on EMBL servers; Schnare et al., J. Mol. Biol, 256: 701-719 (1996); Blundell et al., Nature 326: 347-352 (1987); Fetrow and Bryant, Bio/Technology 11:479-484 (1993); Greer, Methods in Enzymology 202: 239-252 (1991); and Johnson et al, Crit. Rev. Biochem. Mol. Biol. 29:1-68 (1994). An example of homology modeling can be found, for example, in Szklarz G. D., Life Sci. 61: 2507-2520 (1997). These references are incorporated herein by reference.

Thus, in accordance with the present invention, data capable of generating the three-dimensional structure of the above molecules or molecular complexes, or binding pockets thereof, can be stored in a machine-readable storage medium, which is capable of displaying a graphical three-dimensional representation of the structure.

Rational Drug Design

The ACE2 structure coordinates or the three-dimensional graphical representation generated from these coordinates may be used in conjunction with a computer for a variety of purposes, including drug discovery.

For example, the structure encoded by the data may be computationally evaluated for its ability to associate with chemical entities. Chemical entities that associate with ACE2 may inhibit ACE2 or its homologues, and are potential drug candidates. Alternatively, the structure encoded by the data may be displayed in a graphical three-dimensional representation on a computer screen. This allows visual inspection of the structure, as well as visual inspection of the structure's association with chemical entities.

Thus, according to another embodiment, the invention provides a method for designing, selecting and/or optimizing a chemical entity that binds to all or part of the molecule or molecular complex comprising the steps of:

(a) providing the structure coordinates of said molecule or molecular complex on a computer comprising the means for generating three-dimensional structural information of all or part of said molecule or molecular complex from said structure coordinates; and

(b) designing, selecting and/or optimizing said chemical entity by employing means for performing a fitting operation between said chemical entity and said three-dimensional structural information of all or part of said molecule or molecular complex.

In one embodiment, the method is for designing, selecting and or optimizing a chemical entity that binds with the binding pocket of a molecule or molecular complex. In one embodiment, the above method further comprises the following steps before step (a):

(c) producing a crystal of a molecule or molecular complex comprising ACE2 or homologue thereof;

(d) determining the three-dimensional structure coordinates of the molecule or molecular complex by X-ray diffraction of the crystal; and

(e) identifying all or part of said binding pocket.

Three-dimensional structural information in step (a) may be generated by instructions such as a computer program or commands that can generate a three-dimensional structure or graphical representation; subtract distances between atoms; calculate chemical energies for an ACE2 molecule, molecular complex or homologues thereof; or calculate or minimize energies of an association of ACE2 molecule, molecular complex or homologues thereof to a chemical entity. These types of computer programs are known in the art. The graphical representation can be generated or displayed by commercially available software programs. Examples of software programs include but are not limited to QUANTA (Molecular Simulations, Inc., San Diego, Calif. (1998, 2000; Accelrys ©2001, 2002), O (Jones et al., Acta Crystallogr. A47, pp. 110-119 (1991)) and RIBBONS (Carson, J. Appl. Crystallogr., 24, pp. 9589-961 (1991)), which are incorporated herein by reference. Certain software programs may imbue this representation with physico-chemical attributes which are known from the chemical composition of the molecule, such as residue charge, hydrophobicity, torsional and rotational degrees of freedom for the residue or segment, etc. Examples of software programs for calculating chemical energies are described below.

Thus, according to another embodiment, the invention provides a method for evaluating the potential of a chemical entity to associate with all or part of a molecule or molecular complex of this invention as described previously in the different embodiments.

This method comprises the steps of: (a) employing computational means to perform a fitting operation between the chemical entity and all or part of a molecule or molecular complex of this invention; (b) analyzing the results of said fitting operation to quantify the association between the chemical entity and all or part of said molecule or molecular complex; and optionally (c) outputting said quantified association to a suitable output hardware, such as a CRT display terminal, a CD or DVD recorder, ZIP™ or JAZ™ drive, a disk drive, or other machine-readable data storage device, as described previously. The method may further comprise generating a three-dimensional structure, graphical representation thereof, or both of all or part of the molecule or molecular complex prior to step (a). In one embodiment, the method is for evaluating the ability of a chemical entity to associate with all or part of the binding pocket of a molecule or molecular complex of this invention.

In another embodiment, this method comprises the steps of: (a) providing the structure coordinates of the binding pocket or molecule or molecular complex of a protein of this invention, as above-detailed, on a computer comprising the means for generating three-dimensional structural information from the structure coordinates; (b) employing computational means to perform a fitting operation between the chemical entity and all or part of said molecule or molecular complex of this invention described above; (c) analyzing the results of said fitting operation to quantify the association between the chemical entity and all or part of the molecule or molecular complex; and optionally (d) outputting said quantified association to a suitable output hardware, such as a CRT display terminal, a CD or DVD recorder, ZIP™ or JAZ™ drive, a disk drive, or other machine-readable data storage device, as described previously. The method may further comprise generating a three-dimensional structure, graphical representation thereof, or both of all or part of the molecule or molecular complex prior to step (b). In one embodiment, the method is for evaluating the ability of a chemical entity to associate with all or part of the binding pocket of a molecule or molecular complex.

In another embodiment, the invention provides a method for screening a plurality of chemical entities to associate with all or part of a molecule or molecular complex of this invention at a deformation energy of binding of less than −7 kcal/mol with said binding pocket:

(a) employing computational means, which utilize said structure coordinates to perform a fitting operation between one of said chemical entities from the plurality of chemical entities and said binding pocket;

(b) quantifying the deformation energy of binding between the chemical entity and the binding pocket;

(c) repeating steps (a) and (b) for each remaining chemical entity; and

(d) outputting a set of chemical entities that associate with the binding pocket at a deformation energy of binding of less than −7 kcal/mol to a suitable output hardware.

In another embodiment, the method comprises the steps of:

(a) constructing a computer model of a binding pocket of a molecule or molecular complex of this invention;

(b) selecting a chemical entity to be evaluated by a method selected from the group consisting of assembling said chemical entity; selecting a chemical entity from a small molecule database; de novo ligand design of said chemical entity; and modifying a known agonist or inhibitor, or a portion thereof, of an ACE2 protein, or homologue thereof;

(c) employing computational means to perform a fitting operation between computer models of said chemical entity to be evaluated and said binding pocket in order to provide an energy-minimized configuration of said chemical entity in the binding pocket; and

(d) evaluating the results of said fitting operation to quantify the association between said chemical entity and the binding pocket model, whereby evaluating the ability of said chemical entity to associate with said binding pocket.

In another embodiment, the invention provides a method of using a computer for evaluating the ability of a chemical entity to associate with all or part of a molecule or molecular complex of this invention, wherein said computer comprises a machine-readable data storage medium comprising a data storage material encoded with said structure coordinates defining a binding pocket of said molecule or molecular complex and means for generating a three-dimensional graphical representation of the binding pocket, and wherein said method comprises the steps of:

(a) positioning a first chemical entity within all or part of said binding pocket using a graphical three-dimensional representation of the structure of the chemical entity and the binding pocket;

(b) performing a fitting operation between said chemical entity and said binding pocket by employing computational means;

(c) analyzing the results of said fitting operation to quantitate the association between said chemical entity and all or part of the binding pocket; and optionally

(d) outputting said quantitated association to a suitable output hardware.

The above method may further comprise the steps of:

(e) repeating steps (a) through (d) with a second chemical entity; and

(f) selecting at least one of said first or second chemical entity that associates with all or part of said binding pocket based on said quantitated association of said first or second chemical entity.

Alternatively, the structure coordinates of the ACE2 binding pockets may be utilized in a method for identifying an agonist or antagonist of a molecule or molecular complex of this invention comprising a binding pocket of ACE2. This method comprises the steps of:

(a) using a three-dimensional structure of the molecule or molecular complex of this invention to design or select a chemical entity;

(b) contacting the chemical entity with the molecule and molecular complex;

(c) monitoring the activity of the molecule or molecular complex; and

(d) classifying the chemical entity as an agonist or antagonist based on the effect of the chemical entity on the activity of the molecule or molecular complex.

In one embodiment, step (a) is using a three-dimensional structure of the binding pocket of the molecule or molecular complex. In another embodiment, the three-dimensional structure is displayed as a graphical representation.

In another embodiment, the method comprises the steps of:

(a) constructing a computer model of a binding pocket of the molecule or molecular complex;

(b) selecting a chemical entity to be evaluated by a method selected from the group consisting of assembling said chemical entity; selecting a chemical entity from a small molecule database; de novo ligand design of said chemical entity; and modifying a known agonist or inhibitor, or a portion thereof, of an ACE2 protein or homologue thereof;

(c) employing computational means to perform a fitting operation between computer models of said chemical entity to be evaluated and said binding pocket in order to provide an energy-minimized configuration of said chemical entity in the binding pocket; and

(d) evaluating the results of said fitting operation to quantify the association between said chemical entity and the binding pocket model, whereby evaluating the ability of said chemical entity to associate with said binding pocket;

(e) synthesizing said chemical entity; and

(f) contacting said chemical entity with said molecule or molecular complex to determine the ability of said compound to activate or inhibit said molecule.

In one embodiment, the invention provides a method of designing a compound or complex that associates with all or part of the binding pocket of a molecule or molecular complex of this invention comprising the steps of:

(a) providing the structure coordinates of said binding pocket on a computer comprising the means for generating three-dimensional structural information from said structure coordinates;

(b) using the computer to perform a fitting operation to associate a first chemical entity with all or part of the binding pocket;

(c) performing a fitting operation to associate at least a second chemical entity with all or part of the binding pocket;

(d) quantifying the association between the first and second chemical entity and all or part of the binding pocket;

(e) optionally repeating steps (b) to (d) with another first and second chemical entity, selecting a first and a second chemical entity based on said quantified association of all of said first and second chemical entity;

(f) optionally, visually inspecting the relationship of the first and second chemical entity to each other in relation to the binding pocket on a computer screen using the three-dimensional graphical representation of the binding pocket and said first and second chemical entity; and

(g) assembling the first and second chemical entity into a compound or complex that associates with all or part of said binding pocket by model building.

For the first time, the present invention permits the use of molecular design techniques to identify, select and design chemical entities, including inhibitory compounds, capable of binding to ACE2 or ACE2-like binding pockets, motifs and domains.

Applicants' elucidation of binding pockets on ACE2 provides the necessary information for designing new chemical entities and compounds that may interact with ACE2 substrate or binding pockets or ACE2-like substrate or binding pockets, in whole or in part. Due to the homology in the core between ACE2 and homologous molecules, compounds that inhibit ACE2 may also be expected to inhibit these homologous molecules, especially those compounds that bind the binding pocket.

Throughout this section, discussions about the ability of a chemical entity to bind to, associate with or inhibit ACE2 binding pockets refer to features of the entity alone. Assays to determine if a compound binds to ACE2 are well known in the art and are exemplified below.

The design of compounds that bind to or inhibit ACE2 binding pockets according to this invention generally involves consideration of two factors. First, the chemical entity must be capable of physically and structurally associating with parts or all of the ACE2 binding pockets. Non-covalent molecular interactions important in this association include hydrogen bonding, van der Waals interactions, hydrophobic interactions and electrostatic interactions.

Second, the chemical entity must be able to assume a conformation that allows it to associate with the ACE2 binding pockets directly. Although certain portions of the chemical entity will not directly participate in these associations, those portions of the chemical entity may still influence the overall conformation of the molecule. This, in turn, may have a significant impact on potency. Such conformational requirements include the overall three-dimensional structure and orientation of the chemical entity in relation to all or a portion of the binding pocket, or the spacing between functional groups of a chemical entity comprising several chemical entities that directly interact with the ACE2 or ACE2-like binding pockets.

The potential inhibitory or binding effect of a chemical entity on ACE2 binding pockets may be analyzed prior to its actual synthesis and testing by the use of computer modeling techniques. If the theoretical structure of the given entity suggests insufficient interaction and association between it and the ACE2 binding pockets, testing of the entity is obviated. However, if computer modeling indicates a strong interaction, the molecule may then be synthesized and tested for its ability to bind to an ACE2 binding pocket. This may be achieved by testing the ability of the molecule to inhibit ACE2 using the assays described in Example 10. In this manner, synthesis of inoperative compounds may be avoided.

A potential inhibitor of an ACE2 binding pocket may be computationally evaluated by means of a series of steps in which chemical entities or fragments are screened and selected for their ability to associate with the ACE2 binding pockets.

One skilled in the art may use one of several methods to screen chemical entities or fragments for their ability to associate with an ACE2 binding pocket. This process may begin by visual inspection of, for example, an ACE2 binding pocket on the computer screen based on the ACE2 structure coordinates FIG. 1A, 2A, 3A or 3B, or other coordinates which define a similar shape generated from the machine-readable storage medium. Selected fragments or chemical entities may then be positioned in a variety of orientations, or docked, within that binding pocket as defined supra. Docking may be accomplished using software such as QUANTA (Molecular Simulations, Inc., San Diego, Calif. ©1998, 2000; Accelrys ©2001, 2002) and Sybyl (Tripos Associates, St. Louis, Mo.), followed by energy minimization and molecular dynamics with standard molecular mechanics force fields, such as CHARMM and AMBER.

Specialized computer programs may also assist in the process of selecting fragments or chemical entities. These include:

1. GRID (P. J. Goodford, “A Computational Procedure for Determining Energetically Favorable Binding Sites on Biologically Important Macromolecules”, J. Med. Chem., 28, pp. 849-857 (1985)). GRID is available from Oxford University, Oxford, UK.

2. MCSS (A. Miranker et al., “Functionality Maps of Binding Sites: A Multiple Copy Simultaneous Search Method.” Proteins: Structure, Function and Genetics, 11, pp. 29-34 (1991)). MCSS is available from Molecular Simulations, San Diego, Calif.

3. AUTODOCK (D. S. Goodsell et al., “Automated Docking of Substrates to Proteins by Simulated Annealing”, Proteins: Structure, Function, and Genetics, 8, pp. 195-202 (1990)). AUTODOCK is available from Scripps Research Institute, La Jolla, Calif.

4. DOCK (I. D. Kuntz et al., “A Geometric Approach to Macromolecule-Ligand Interactions”, J. Mol. Biol., 161, pp. 269-288 (1982)). DOCK is available from University of California, San Francisco, Calif.

Once suitable chemical entities or fragments have been selected, they can be assembled into a single compound or complex. Assembly may be preceded by visual inspection of the relationship of the fragments to each other on the three-dimensional image displayed on a computer screen in relation to the structure coordinates of ACE2. This would be followed by manual model building using software such as QUANTA (Molecular Simulations, Inc., San Diego, Calif. ©1998, 2000; Accelrys ©2001, 2002) or Sybyl (Tripos Associates, St. Louis, Mo.).

Useful programs to aid one of skill in the art in connecting the individual chemical entities or fragments include:

1. CAVEAT (P. A. Bartlett et al., “CAVEAT: A Program to Facilitate the Structure-Derived Design of Biologically Active Molecules”, in Molecular Recognition in Chemical and Biological Problems, Special Pub., Royal Chem. Soc., 78, pp. 182-196 (1989); G. Lauri and P. A. Bartlett, “CAVEAT: a Program to Facilitate the Design of Organic Molecules”, J. Comput. Aided Mol. Des., 8, pp. 51-66 (1994)). CAVEAT is available from the University of California, Berkeley, Calif.

2. 3D Database systems such as ISIS (MDL Information Systems, San Leandro, Calif.). This area is reviewed in Y. C. Martin, “3D Database Searching in Drug Design”, J. Med. Chem., 35, pp. 2145-2154 (1992).

3. HOOK (M. B. Eisen et al., “HOOK: A Program for Finding Novel Molecular Architectures that Satisfy the Chemical and Steric Requirements of a Macromolecule Binding Site”, Proteins: Struct., Funct., Genet., 19, pp. 199-221 (1994)). HOOK is available from Molecular Simulations, San Diego, Calif.

Instead of proceeding to build an inhibitor of an ACE2 binding pocket in a step-wise fashion one fragment or chemical entity at a time as described above, inhibitory or other ACE2 binding compounds may be designed as a whole or “de novo” using either an empty binding pocket or optionally including some portion(s) of a known inhibitor(s). There are many de novo ligand design methods including:

1. LUDI (H.-J. Bohm, “The Computer Program LUDI: A New Method for the De Novo Design of Enzyme Inhibitors”, J. Comp. Aid. Molec. Design, 6, pp. 61-78 (1992)). LUDI is available from Molecular Simulations Incorporated, San Diego, Calif.

2. LEGEND (Y. Nishibata et al., Tetrahedron, 47, p. 8985 (1991)). LEGEND is available from Molecular Simulations Incorporated, San Diego, Calif.

3. LeapFrog (available from Tripos Associates, St. Louis, Mo.).

4. SPROUT (V. Gillet et al., “SPROUT: A Program for Structure Generation)”, J. Comput. Aided Mol. Design, 7, pp. 127-153 (1993)). SPROUT is available from the University of Leeds, UK.

Other molecular modeling techniques may also be employed in accordance with this invention (see, e.g., N. C. Cohen et al., “Molecular Modeling Software and Methods for Medicinal Chemistry, J. Med. Chem., 33, pp. 883-894 (1990); see also, M. A. Navia and M. A. Murcko, “The Use of Structural Information in Drug Design”, Current Opinions in Structural Biology, 2, pp. 202-210 (1992); L. M. Balbes et al., “A Perspective of Modern Methods in Computer-Aided Drug Design”, Reviews in Computational Chemistry, Vol. 5, K. B. Lipkowitz and D. B. Boyd, Eds., VCH, New York, pp. 337-380 (1994); see also, W. C. Guida, “Software For Structure-Based Drug Design”, Curr. Opin. Struct. Biology, 4, pp. 777-781 (1994)).

Once a chemical entity has been designed or selected by methods described above, the efficiency with which that chemical entity may bind to an ACE2 binding pocket may be tested and optimized by computational evaluation. For example, an effective ACE2 binding pocket inhibitor must preferably demonstrate a relatively small difference in energy between its bound and free states (i.e., a small deformation energy of binding). Thus, the most efficient ACE2 binding pocket inhibitors should preferably be designed with a deformation energy of binding of not greater than about 10 kcal/mole, more preferably, not greater than 7 kcal/mole. ACE2 binding pocket inhibitors may interact with the binding pocket in more than one conformation that is similar in overall binding energy. In those cases, the deformation energy of binding is taken to be the difference between the energy of the free chemical entity and the average energy of the conformations observed when the inhibitor binds to the protein.

A chemical entity designed or selected as binding to an ACE2 binding pocket may be further computationally optimized so that in its bound state it would preferably lack repulsive electrostatic interaction with the target enzyme and with the surrounding water molecules. Such non-complementary electrostatic interactions include repulsive charge-charge, dipole-dipole and charge-dipole interactions.

Specific computer software is available in the art to evaluate compound deformation energy and electrostatic interactions. Examples of programs designed for such uses include: Gaussian 94, revision C (M. J. Frisch, Gaussian, Inc., Pittsburgh, Pa. ©1995); AMBER, version 4.1 (P. A. Kollman, University of California at San Francisco, ©1995); QUANTA/CHARMM (Molecular Simulations, Inc., San Diego, Calif. ©1998, 2000; Accelrys ©2001, 2002); Insight II/Discover (Molecular Simulations, Inc., San Diego, Calif. ©1998); DelPhi (Molecular Simulations, Inc., San Diego, Calif. ©1998); and AMSOL (Quantum Chemistry Program Exchange, Indiana University). These programs may be implemented, for instance, using a Silicon Graphics workstation such as an Indigo2 with “IMPACT” graphics. Other hardware systems and software packages will be known to those skilled in the art.

Another approach enabled by this invention is the computational screening of small molecule databases for chemical entities or compounds that can bind in whole or in part to an ACE2 binding pocket. In this screening, the quality of fit of such entities to the binding pocket may be judged either by shape complementarity or by estimated interaction energy (E. C. Meng et al., J. Comp. Chem., 13, pp. 505-524 (1992)).

According to another embodiment, the invention provides compounds which associate with an ACE2 binding pocket produced or identified by the method set forth above.

Another particularly useful drug design technique enabled by this invention is iterative drug design. Iterative drug design is a method for optimizing associations between a protein and a compound by determining and evaluating the three-dimensional structures of successive sets of protein/compound complexes.

In iterative drug design, crystals of a series of protein or protein complexes are obtained and then the three-dimensional structures of each crystal is solved. Such an approach provides insight into the association between the proteins and compounds of each complex. This is accomplished by selecting compounds with inhibitory activity, obtaining crystals of this new protein/compound complex, solving the three-dimensional structure of the complex, and comparing the associations between the new protein/compound complex and previously solved protein/compound complexes. By observing how changes in the compound affected the protein/compound associations, these associations may be optimized.

In some cases, iterative drug design is carried out by forming successive protein-compound complexes and then crystallizing each new complex. High throughput crystallization assays may be used to find a new crystallization condition or to optimize the original protein or complex crystallization condition for the new complex. Alternatively, a pre-formed protein crystal may be soaked in the presence of an inhibitor, thereby forming a protein/compound complex and obviating the need to crystallize each individual protein/compound complex.

Structure Determination of Other Molecules

The structure coordinates set forth in FIGS. 1A, 2A, 3A or 3B can also be used in obtaining structural information about other crystallized molecules or molecular complexes. This may be achieved by any of a number of well-known techniques, including molecular replacement.

According to one embodiment of this invention, the machine-readable data storage medium comprises a data storage material encoded with a first set of machine readable data which comprises the Fourier transform of at least a portion of the structure coordinates set forth in FIGS. 1A, 2A, 3A or 3B or homology model thereof, and which, when using a machine programmed with instructions for using said data, can be combined with a second set of machine readable data comprising the X-ray diffraction pattern of a molecule or molecular complex to determine at least a portion of the structure coordinates corresponding to the second set of machine readable data.

In another embodiment, the invention provides a computer for determining at least a portion of the structure coordinates corresponding to X-ray diffraction data obtained from a molecule or molecular complex, wherein said computer comprises:

(a) a machine-readable data storage medium comprising a data storage material encoded with machine-readable data, wherein said data comprises at least a portion of the structure coordinates of ACE2 according to FIGS. 1A, 2A, 3A or 3B or homology model thereof;

(b) a machine-readable data storage medium comprising a data storage material encoded with machine-readable data, wherein said data comprises X-ray diffraction data obtained from said molecule or molecular complex; and

(c) instructions for performing a Fourier transform of the machine-readable data of (a) and for processing said machine-readable data of (b) into structure coordinates.

For example, the Fourier transform of at least a portion of the structure coordinates set forth in FIGS. 1A, 2A, 3A or 3B or homology model thereof may be used to determine at least a portion of the structure coordinates of ACE2 homologues. In one embodiment, the molecule is an ACE2 homologue. In another embodiment, the molecular complex is selected from the group consisting of ACE2 complex and ACE2 homologue complex.

Therefore, in another embodiment this invention provides a method of utilizing molecular replacement to obtain structural information about a molecule or a molecular complex of unknown structure wherein the molecule or molecular complex is sufficiently homologous to ACE2, comprising the steps of:

(a) crystallizing said molecule or molecular complex of unknown structure;

(b) generating an X-ray diffraction pattern from said crystallized molecule or molecular complex;

(c) applying at least a portion of the ACE2 structure coordinates set forth in one of FIGS. 1A, 2A, 3A or 3B or a homology model thereof to the X-ray diffraction pattern to generate a three-dimensional electron density map of at least a portion of the molecule or molecular complex whose structure is unknown; and

(d) generating a structural model of the molecule or molecular complex from the three-dimensional electron density map.

In one embodiment, the method is performed using a computer. In another embodiment, the molecule is selected from the group consisting of ACE2 and ACE2 homologues. In another embodiment, the molecule is an ACE2 molecular complex or homologue thereof.

By using molecular replacement, all or part of the structure coordinates of ACE2 as provided by this invention or homology model thereof (and set forth in FIGS. 1A, 2A, 3A or 3B) can be used to determine the structure of a crystallized molecule or molecular complex whose structure is unknown more quickly and efficiently than attempting to determine such information ab initio.

Molecular replacement provides an accurate estimation of the phases for an unknown structure. Phases are a factor in equations used to solve crystal structures that can not be determined directly. Obtaining accurate values for the phases, by methods other than molecular replacement, is a time-consuming process that involves iterative cycles of approximations and refinements and greatly hinders the solution of crystal structures. However, when the crystal structure of a protein containing at least a homologous portion has been solved, the phases from the known structure may provide a satisfactory estimate of the phases for the unknown structure.

Thus, this method involves generating a preliminary model of a molecule or molecular complex whose structure coordinates are unknown, by orienting and positioning the relevant portion of ACE2 protein according to FIGS. 1A, 2A, 3A or 3B within the unit cell of the crystal of the unknown molecule or molecular complex so as best to account for the observed X-ray diffraction pattern of the crystal of the molecule or molecular complex whose structure is unknown. Phases can then be calculated from this model and combined with the observed X-ray diffraction pattern amplitudes to generate an electron density map of the structure whose coordinates are unknown. This, in turn, can be subjected to any well-known model building and structure refinement techniques to provide a final, accurate structure of the unknown crystallized molecule or molecular complex (E. Lattman, “Use of the Rotation and Translation Functions”, in Meth. Enzymol., 115, pp. 55-77 (1985); M. G. Rossmann, ed., “The Molecular Replacement Method”, Int. Sci. Rev. Ser., No. 13, Gordon & Breach, New York (1972)).

The structure of any portion of any crystallized molecule or molecular complex that is sufficiently homologous to any portion of the structure of human ACE2 protein which is solved and provided herein can be resolved by this method.

In one embodiment, the method of molecular replacement is utilized to obtain structural information about an ACE2 homologue. The structure coordinates of ACE2 as provided by this invention are particularly useful in solving the structure of ACE2 complexes that are bound by ligands, substrates and inhibitors.

Furthermore, the structure coordinates of ACE2 as provided by this invention are useful in solving the structure of ACE2 proteins that have amino acid substitutions, additions and/or deletions (referred to collectively as “ACE2 mutants”, as compared to naturally occurring ACE2). These ACE2 mutants may optionally be crystallized in co-complex with a chemical entity, such as a non-hydrolyzable ATP analogue or a suicide substrate. The crystal structures of a series of such complexes may then be solved by molecular replacement and compared with that of wild-type ACE2. Potential sites for modification within the various binding pockets of the enzyme may thus be identified. This information provides an additional tool for determining the most efficient binding interactions, for example, increased hydrophobic interactions, between ACE2 and a chemical entity or compound.

The structure coordinates are also particularly useful in solving the structure of crystals of ACE2 or ACE2 homologues co-complexed with a variety of chemical entities. This approach enables the determination of the optimal sites for interaction between chemical entities, including candidate ACE2 inhibitors. For example, high resolution X-ray diffraction data collected from crystals exposed to different types of solvent allows the determination of where each type of solvent molecule resides. Small molecules that bind tightly to those sites can then be designed and synthesized and tested for their ACE2 inhibition activity.

All of the complexes referred to above may be studied using well-known X-ray diffraction techniques and may be refined using 1.5-3.4 Å resolution X-ray data to an R value of about 0.30 or less using computer software, such as X-PLOR (Yale University, ©1992, distributed by Molecular Simulations, Inc.; see, e.g., Blundell & Johnson, supra; Meth. Enzymol., vol. 114 & 115, H. W. Wyckoff et al., eds., Academic Press (1985)) or CNS (Brunger et al., Acta Cryst., D54, pp. 905-921, (1998)).

All references cited herein are incorporated by reference.

In order that this invention be more fully understood, the following examples are set forth. These examples are for the purpose of illustration only and are not to be construed as limiting the scope of the invention in any way.

### EXAMPLE 1

### ACE2 Expression and Purification

An expression vector was generated encoding a secreted form of human ACE2 (amino acids 1-740) in the pBac Pak9 vector (Clontech, Palo Alto, Calif.). This secreted construct was prepared by inserting a stop codon right after Ser 740, which precedes the predicted transmembrane domain (Donoghue et al., supra). Thus, the transmembrane domain and the cytosolic domain (residues 741 to 805) were not expressed when this expression vector bearing ACE2 was introduced into cells. Presumably the signal sequence (residues 1 to 18 of human ACE2) is also removed upon secretion from SF9 cells. The molecular weight of the purified enzyme was found to be 89.6 kDa by MALDI-TOF mass spectrometry, which is greater than the theoretical molecular weight of 83.5 kDa expected from the primary sequence (residues 19 to 740). The difference of about 6 kDa is believed to be due to glycosylation at the seven predicted N-linked glycosylation sites for this protein (at amino acid residues N53, N90, N103, N322, N432, N546 and N690).

The truncated extracellular form of human ACE2 (residues 1 to 740) was expressed in baculovirus expression system and purified (Vickers et al, supra). Specifically, SF9 cells were infected at multiplicity of infection of 0.1 with ACE2 baculovirus (i.e., baculovirus vector bearing human ACE2; said vector expresses human ACE2 1-740 in permissive cells) of a titer of 1.1×10⁹ pfu/ml. A 10 L fermentation run was carried out with SF9 cells grown to 1.3×10⁶ cells/ml in SF900II SFM (Gibco/Life Technologies), 18 mM L-Glutamine, and IX antibiotic-antimycotic (from 100× stock Gibco/Life Technologies) at 27° C. At 96 h post infection, cells were pelleted at 5000×g centrifugation, and the culture supernatant was collected, frozen, and stored at −80° C.

The thawed supernatant was filtered (0.2 μm filter), loaded onto a Toyopearl QAE anion exchanger column, and the column washed with buffer A (25 mM Tris HCl, pH 8.0). A 0-50% gradient elution was then performed with increasing buffer B (1.0 M NaCl, 25 mM Tris HCl, pH 8.0) using a total of 5 column volumes. The ACE2 containing fractions, as detected by Coomassie-stained SDS-PAGE, were pooled and (NH₄)₂SO₄ was added to a final concentration of 1.0 M. The sample was then loaded onto a Toyopearl Phenyl column. After loading, the column was washed with buffer C (1.0 M (NH₄)₂SO₄, 25 mM Tris HCl, pH 8.0) using 5 column volumes, and then gradient eluted with buffer A (0-100%). The ACE2 containing fractions, as detected by Coomassie-stained SDS-PAGE, were pooled and dialyzed against buffer A at 4° C. overnight. The dialyzed ACE2 protein sample was sequentially loaded onto MonoQ column (Pharmacia, Piscataway, N.J.), and gradient eluted with buffer B. The ACE2 containing fractions from the MonoQ column, as detected by Coomassie-stained SDS-PAGE, were concentrated with a Centricon (Millipore Corp., Bedford, Mass.) concentrator, mw cutoff 30 kD. The concentrated sample was loaded onto an TSK G3000SW×l size exclusion column, and eluted with buffer A.

The above-described expression and purification method leads to protein estimated to be more than 90% pure.

### EXAMPLE 2

### Protein Crystallization for Native ACE2

Purified human ACE2 protein from Example 1 was concentrated to approximately 5 mg/ml and set up for crystallization using hanging drop vapor diffusion methods at 16 to 18° C. 2 μl of concentrated purified ACE2 was combined with 2 μl of reservoir solution. Initial crystals of ACE2 were obtained using the Crystal Screen and Crystal Screen 2 crystallization screening kits (Hampton Research; Laguna Niguel, Calif.). Subsequently, a PEG/Ion screen (Hampton Research) was used to further explore and optimize the ACE2 crystallization process. The crystallization reservoir solution conditions for native ACE2 were found to be 100 mM Tris-HCl pH 8.5, 200 mM MgCl₂, 13 or 14% PEG 8000 at 16 to 18° C. The best crystallization reservoir solution conditions for native ACE2 were found to be 100 mM Tris-HCl pH 8.5, 200 mM MgCl₂, 14% PEG 8000 at 16 to 18° C. Under these conditions it took about two weeks to grow single crystals suitable for X-ray diffraction.

### EXAMPLE 3

### Protein Crystallization for ACE2 Complexes

Diffraction quality crystals of human ACE2 protein from Example 1 in complex with inhibitor, 2, 3 and 4 grew under crystallization conditions of 15-20% PEG 8000, 400-800 mM NaCl and 100 mM Tris-HCl pH 7.5 or 18-22% PEG 2000, 400-600 mM NaCl and 100 mM Tris-HCl pH 7.0. Complex crystals also grew in PEG 4000. ACE2-inhibitor1 crystals used for X-ray diffraction were grown under 19% PEG 3000, 100 mM Tris pH 7.5 and 600 mM NaCl. ACE2-inhibitor2 crystals used for X-ray diffraction were grown under 25% PEG 2000, 100 mM Tris pH 7.0 and 300 mM NaCl. ACE2-inhibitor3 crystals used for X-ray diffraction were grown under 18% PEG 8000, 100 mM Tris pH 7.5 and 600 mM NaCl. ACE2-inhibitor4 crystals used for X-ray diffraction were grown under 20% PEG 8000, 100 mM Tris pH 7.5 and 600 mM NaCl. Crystallization setups contained 2 μl reservoir solution, 2 μl 5.9 mg/ml ACE2 (139 pmol) and 0.2 μl of 1.0 mM inhibitor (200 pmol, final inhibitor concentration is about 48 μM).

Diffraction quality ACE2 crystals were grown in the presence of an ACE2 inhibitor1 ((S, S) 2-{1-carboxy-2-[3-(3,5-dichloro-benzyl)-3H-imidazol-4-yl]-ethylamino}-4-methyl-pentanoic acid), which corresponds to compound 16 in Table 1 of Dales et al., supra, which is incorporated herein by reference. The best diffracting ACE2-inhibitor1 complex crystals were grown in the presence of 19% PEG 3000, 100 mM Tris pH 7.5 and 600 mM NaCl. Crystallization trials used 211 reservoir solution, 2 μl 5.9 mg/ml ACE2 containing 0.1 mM inhibitor.

### EXAMPLE 4

### X-ray Diffraction and Structure Determination of ACE2

Many of the crystals from Example 2 were found to diffract X-rays in the 2.1 to 5 Å resolution range when screened with synchrotron X-ray radiation at beamline sector 32 COM-CAT at the Advanced Proton Source (APS) at Argonne National Labs (ANL), or the X25 beamline at National Synchrotron Light Source (NSLS) at Brookhaven National Labs (BNL). The best data set for native ACE2 was at 2.2 Å resolution and was collected at the APS at ANL. The space group for this crystal was found to be C2 (monoclinic) with unit cell dimensions of a=103.749 Å b=89.59 Å, c=112.356 Å, with α=γ90.00°, and β=109.124° yielding a unit cell volume of about 986854 Å³. Assuming a molecular weight of about 89 kDa, and four asymmetric units in the unit cell, there was one molecule per asymmetric unit in the crystal lattice. The space group for all of the native ACE2 data sets collected (including the heavy atom derivatives) were C2, although a significant amount of non-isomorphism was observed.

A summary of the X-ray data sets collected for ACE2 are listed in Table 1. The data sets for each derivative were collected at different wavelengths in order to maximize the anomalous signals for the bound heavy atoms. The native data was collected to 2.2 Å resolution at 1.28 Å wavelength in order to maximize the anomalous signal of the Zn atom.

The heavy atom positions were determined and confirmed by a combination of visual inspection of Patterson maps, automatic search procedures which included SHAKE N'BAKE (Hauptman, Methods Enzymol. 277, pp. 3-13. (1997)) and SHELXD (Abrahams and DeGraaff, Curr. Opin. Struct Biol. 8, pp. 601-605 (1998)). The heavy atom parameters were refined and optimized by SHARP (Bricogne, Methods Enzymol. 276, pp. 361-423 (1997)), MLPHARE (Otwinowski, Proceedings of the CCP4 Study Weekend 25-26, pp. 80-86 (1991), Wolf, Evans, and Leslie, Eds.) and XHEAVY (McRee, Practical Protein Crystallography, (1999) 2nd Edition, Academic Press, San Diego, Calif.). The experimental phases were improved by solvent flattening and histogram matching. The resultant computed maps were compared for quality and traceability. The phases obtained form SHARP were of sufficient quality that enabled model building. The model for the ACE2 structure has an Rfactor=23.8% and R_{free}=28.9% for data of 2.2 Å.

### EXAMPLE 5

### X-ray Diffraction and Structure Determination for ACE2 Complexes

One of the co-crystals of inhibitor1 and ACE2 from Example 3 was found to diffract to 2.7 Å resolution. Data was collected at the X25 beamline at the National Synchrotron Light Source (NSLS) at Brookhaven National Labs (BNL). X-ray diffraction data was also obtained for the three other inhibitor/ACE2 complexes at 3.0 to 3.4 Å.

The native ACE2 structure, once determined, was used as a model to solve the inhibitor-bound ACE2 structure to 3.3 Å resolution using the molecular replacement program AmoRe in the CCP4 suite of programs (Navaza, Acta Cryst. A50, pp. 157-163 (1994); Navaza and Saludjian, Methods Enzymol. 276, pp. 581-594 (1997); Brunger, Methods Enzymol. 276, pp. 558-580 (1997)). The native structure was split into two subdomains: subdomain I (residues 19-102, 290-397, and 417-430), and subdomain II (residues 103-289, 398-416, and 431-613). Subdomain II was used for molecular replacement and refined with REFMAC5 (Murshudov et al., “Application of Maximum Likelihood Refinement” in the Refinement of Protein structures, Proceedings of Daresbury Study Weekend (1996)) which resulted in the appearance of electron density for subdomain I. Subdomain I was then fitted into the density by hand and the structure, as a whole, was refined.

### EXAMPLE 6

### Primary Sequence Alignments

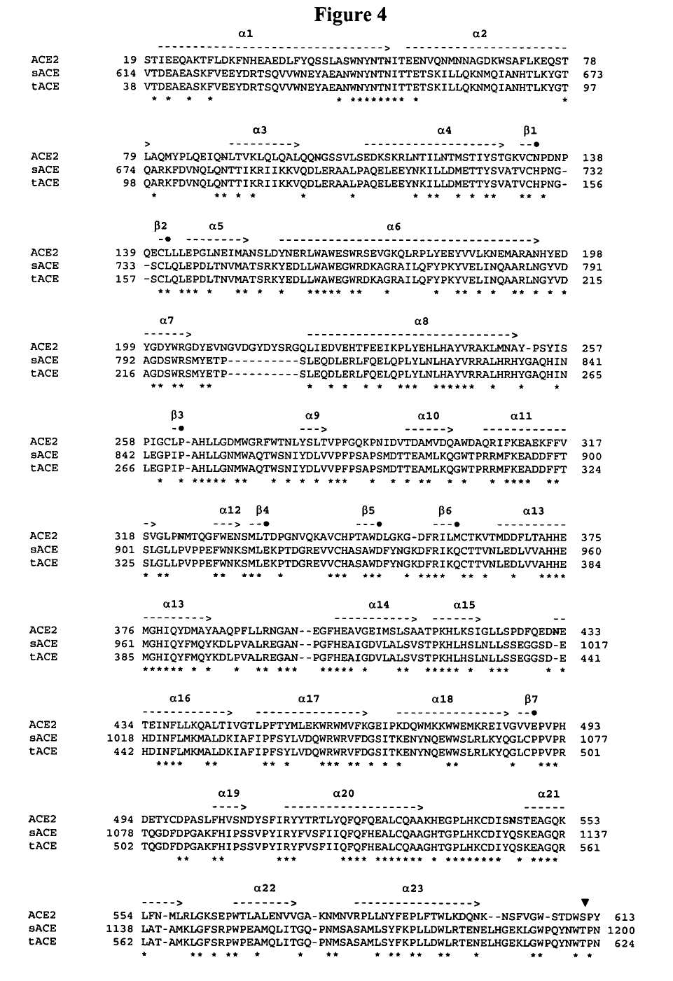

Sequence alignment for the mature extracellular domains of human ACE2, the C-terminal catalytic domain of human somatic ACE (sACE), and human germinal or testicular ACE (tACE) is shown in FIG. 4. The closest homologues of ACE2 were found to be the C-terminal catalytic domain of human somatic ACE, human germinal ACE and N-terminal catalytic domain of human somatic ACE, with 42%, 42% and 41% sequence identity over 616 residues, respectively. The catalytic domain of human germinal ACE is identical to the C-terminal catalytic domain of somatic ACE. Rat neurolysin (Brown et al., Proc. Natl. Acad. Sci. USA 98, pp. 3127-3132 (2001)) has only about 17% sequence identity over 510 residues, and is therefore not shown. The conserved HEXXH motif, which is characteristic of zinc binding sites in metalloproteases, is conserved in all three proteins. The catalytically important residue H1089 of somatic ACE (Fernandez et al., J. Biol. Chem. 276, pp. 4998-5004 (2001)) is conserved in ACE2 (H505) and neurolysin. The R1098 residue of ACE, which is implicated in anion activation (Liu et al., J. Biol. Chem. 276, pp. 33518-525 (2001)), is conserved in ACE2 (R514) but not in neurolysin.

### EXAMPLE 7

### Native ACE2 Structure: Overview of ACE2 Structure

The extracellular region of the native human ACE2 enzyme is comprised of two domains. A metallopeptidase domain (residues 19 to 611) contains the single catalytic Zn-binding motif component, HEXXH, of the ACE2 enzyme (FIG. 4). The second domain is located near the C-terminus (residues 612 to 740) and is about 48% homologous to human Collectrin, a kidney collecting duct-specific glycoprotein (Zhang et al., J. Biol. Chem. 276, pp. 17122-17139 (2001)). The electron density map for the second domain was weak in both the native and complexed ACE2 structures: thus, this region has been excluded from the structural models presented herein.

The metallopeptidase domain is comprised of two subdomains (I and II) (FIGS. 4A and 4B) which form two sides of a long and deep canyon with approximate dimensions of 40 Å long×15 Å wide×25 Å deep. The two catalytic subdomains are connected only at the floor of the active site cleft. One prominent α-helix (helix 20; residues 514 to 533) connects the two domains and forms part of the floor of the canyon.

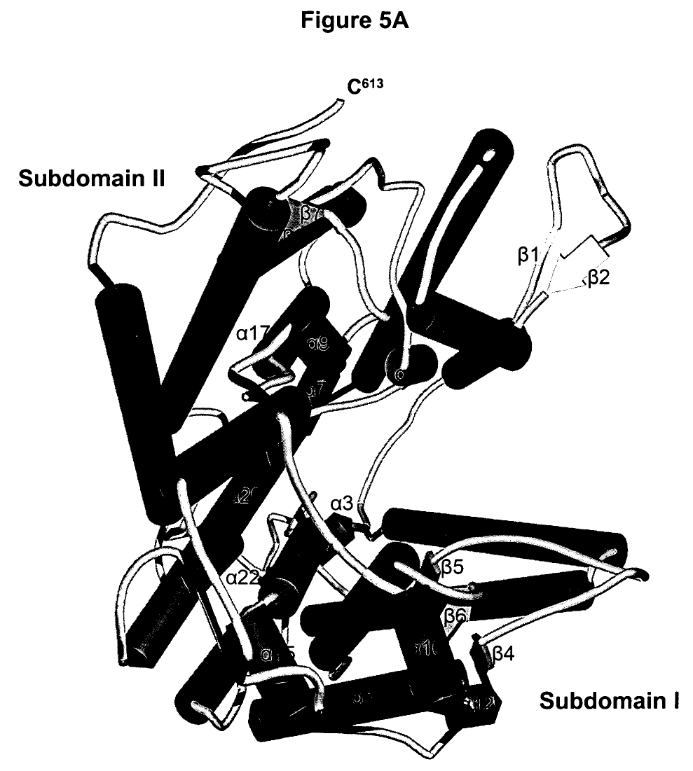

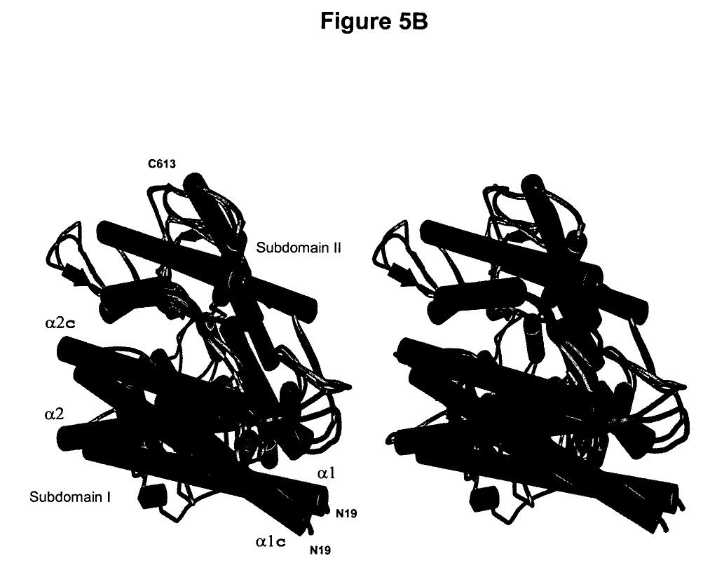

The secondary structure of the metallopeptidase domain of ACE2 (residues 19-613) is comprised of 23 α-helical segments that make up about 59% of the structure (FIG. 5A). Seven short beta strand structural elements make up only about 3.2%.

Glycosylation Sites

There are seven potential N-linked glycosylation sites in the extracellular domain of human ACE2 (residues 19 to 740): N53, N90, N103, N322, N432, N546 and N690 (Tipnis et al., supra). Six of these sites occur in the metallopeptidase domain of ACE2. In the present invention, electron density, which accommodated N-acetyl glucosamine (NAG) groups, was observed at all six positions: N53, N90, N103, N322, N432 and N546, strongly suggesting glycosylation at these positions.

Disulfide Linkages

There are three disulfide bonds in human ACE2 (C133/C141, C344/C361 and C530/C542). All six of these cysteines are conserved in the C-terminal domain of sACE and tACE (FIG. 4). The homologous disulfide linkages correspond to C728/C734, C928/C946 and C1114/C1126 in the C-terminal domain of somatic ACE.

Zinc Binding Site

The zinc binding site is located near the bottom and on one side of the large active site cleft (subdomain I side), nearly midway along the length of the cleft (about 20 Å from either end). The zinc is coordinated by H374, H378, E402 and one water molecule (in the native structure). This Zn-bound water is also hydrogen bonded to E375, which enhances its nucleophilic role in peptide bond hydrolysis, as described for other well characterized zinc metalloproteases (Matthews, Acc. Chem. Res. 21, pp. 333-340 (1988)). These residues at the zinc binding site of ACE2 make up the HEXXH+E motif which is conserved in the zinc metallopeptidase clan MA (Rawlings and Barrett, Methods Enzymol. 248, pp. 183-228 (1995)).

### EXAMPLE 8

### Predictions of the ACE2 Active Site From the Native ACE2 Structure with No Bound Inhibitors

The native human ACE2 structure from Examples 4 and 7 (FIG. 1A) reveals an active site cleft between subdomain II and subdomain I of the metallopeptidase domain. The residues that are present in this cleft and homologous to the C-terminal domain of human somatic ACE and human germinal ACE are H374, E375, E402, H401, H505, R514 and Y515. The residues that are present in the cleft but are unique to ACE2 (different from human sACE or tACE) are E406, R518, Y510, R273, and F274. These later residues are expected to be responsible for many of the observed substrate specificity and inhibitor binding differences for ACE2 compared with somatic ACE.

Deeply recessed and shielded proteolytic active sites are a common structural feature in nature, presumably as a way to avoid hydrolysis of correctly folded and functional proteins. The ACE2 structural homologs, neurolysin (Brown et al., supra) and the P. furiosus carboxypeptidase (Arndt et al., supra) also use this long and deep active site cleft architecture for limiting access. However, other structurally distinct mechanisms for restricting access to proteolytic sites can also be found in the β-propeller motif of the prolyl oligopeptidase (Fülöp et al., Cell 94, pp. 161-170 (1998)), the twisted superstructure of tripeptidyl peptidase II (Rockel et al., EMBO J. 21, pp. 5979-5984 (2002)), and the more complex gated barrel architecture of the 20S (Groll et al., Nature 386, pp. 463-471 (1997); Unno et al, Structure 10, pp. 609-618 (2002)) and 26S proteasome. In all cases, only peptides and partially unfolded proteins with little or no secondary structure have access to these shielded and compartmentalized proteolytic active sites. The deeply recessed active site of ACE2 and ACE is also consistent with the observed requirement of at least 28 Å long spacer arm groups for the affinity purification of somatic ACE (Pantoliano et al., Biochemistry 23, pp. 1037-1042 (1984)).

There is a clear difference between the native ACE2 structure and the inhibitor-bound ACE2 structures with respect to the distance separating the two subdomains (FIG. 5B). These two subdomains were found to undergo a large inhibitor dependent hinge bending movement of one catalytic subdomain relative to the other that results in the complete envelopment of the inhibitor.

Although a conformational change occurs upon inhibitor binding, the native ACE structure from Examples 4 and 7 (FIG. 1A) was used to predict the important binding residues for the complex before the complex structure was finalized. The following paragraphs discuss the specific predictions of the important binding residues.

P1 Substrate Binding Site

There are no substrates, inhibitors, or transition state analogs bound to ACE2 in the native structure. However, it was possible to dock in a tetra-peptide fragment of the substrate angiotesin II (Ile-His-Pro-Phe) into the ACE2 active site in such a way that Phe sits in the P1′ site and Pro sits in the P1 site (docking trials were performed with the docking software package FLO (Colin McMartin at ThistleSoft)). This orientation was taken from that seen in many other zinc metalloprotease x-ray structures that have transition state analogs or other inhibitors bound at the active site (Matthews, B. W., supra and Oefner et al., J. Mol. Biol. 296, pp. 341-349 (2000)). The following 120 residues of human ACE2 line the active site cleft: Phe 40, Ser 44, Trp 69, Ser 70, Leu 73, Lys 74, Ser 77, Thr 78, Leu 85, Leu 91, Thr 92, Lys 94, Leu 95, Gln 96, Gln 98, Ala 99, Leu 100, Gln 101, Gln 102, Asn 103, Gly 104, Ser 106, Asn 194, His 195, Tyr 196, Tyr 199, Tyr 202, Trp 203, Arg 204, Gly 205, Asp 206, Tyr 207, Glu 208, Val 209, Asn 210, Val 212, Arg 219, Arg 273, Phe 274, Thr 276, Tyr 279, Pro 289, Asn 290, Ile 291, Cys 344, His 345, Pro 346, Thr 347, Ala 348, Trp 349, Asp 350, Leu 351, Gly 352, Cys 361, Met 366, Asp 367, Asp 368, Leu 370, Thr 371, His 374, Glu 375, His 378, Asp 382, Tyr 385, Phe 390, Leu 391, Leu 392, Arg 393, Asn 394, Gly 395, Ala 396, Asn 397, Glu 398, Gly 399, Phe 400, His 401, Glu 402, Ala 403, Glu 406, Ser 409, Leu 410, Ala 413, Thr 414, Pro 415, Leu 418, Phe 428, Glu 430, Asp 431, Asn 432, Thr 434, Glu 435, Asn 437, Phe 438, Lys 441, Gln 442, Thr 445, Ile 446, Thr 449, Leu 450, Arg 460, Phe 504, His 505, Ser 507, Asn 508, Asp 509, Tyr 510, Ser 511, Arg 514, Tyr 515, Arg 518, Thr 519, Gln 522, His 540, Lys 541, Lys 562, Ser 563, Glu 564, Pro 565, Trp 566, Tyr 587.

The residues that are in the vicinity of the P1 binding site of ACE2 can be determined from the above tetra-peptide docking results. These residues are Thr 347, Glu 402, Pro 346 on the Zn side of the active site cleft (subdomain I), and Tyr 515 and Arg 514, Tyr 510, and Phe 504 on the opposing face of the cleft (subdomain II). Although these residues on the opposite side of the cleft (subdomain II) from the zinc site are about 10 Å away from the P1 proline of the modeled peptide model, they could possibly interact with the substrate P1 site if there is a conformational change upon substrate (or inhibitor) binding that brings subdomain II closer to subdomain I. Only three of these residues near the P1 site are different in somatic ACE: Y510V, P346A and T347S. Y510 of ACE2 is also a tyrosine in neurolysin.

P1′ Substrate Binding Site

The residues that are in the vicinity of the P1′ binding site of native ACE2 can also be determined from the tetra-peptide docking results discussed above. These residues are His 345, Pro 346, Thr 371, His 374, Glu 406, Arg 518, and Ser 409 on the zinc face of the active site cleft. On the opposing face of the cleft are residues Phe 274, and Arg 273. All of these residues differ from corresponding somatic ACE amino acid residues except for H345 and H374 (P346->A, T371->V, E406->D, R518->S, F274->T and R273->Q, S409->A) (the identity of the ACE2 residue is listed first; its position is indicated using ACE2 sequence numbering; and the identity of the sACE residue is given at the end). These collective differences between ACE2 and ACE are presumably responsible for the substrate specificity switch from dipeptidyl carboxypeptidase activity in ACE to carboxypeptidase-like activity in ACE2.

The three most noteworthy residues at the P1′ binding site of ACE2 are R518, E406 and R273. Two noteworthy residues at the P1′ binding site of ACE2 are R518 and E406, which are Ser and Asp residues, respectively, in the somatic ACE C-terminal domain. The fact that they are both different in ACE2 when compared to sACE suggests that they play an important role in the substrate specificity differences between ACE2 and ACE enzymes.

R518 and E406 interact with each other through a salt bridge in ACE2. The R518 residue is particularly important since it is at a position that is analogous to that of R145 in carboxypeptidase A (CPA). R145 has been shown to play an important role in substrate recognition for CPA where it forms hydrogen bonds with the C-terminal carboxylate of substrates and inhibitors (Christianson & Lipscomb, Acc. Chem. Res. 22, pp. 62-69 (1989)). The corresponding residue in somatic and germinal ACE is a serine. Thus, this change from serine in ACE to arginine in ACE2 could possibly explain the change from a dipeptidyl carboxypeptidase activity in ACE to the observed CPA-like activity for ACE2 (Donoghue et al., supra; Tipnis et al., supra; Vickers et al., supra).

The E406 residue of ACE2 corresponds to D991 of somatic ACE. This residue was the subject of site directed mutagenesis studies for somatic ACE (Williams et al., J. Biol. Chem. 269, pp. 29430-434 (1994)). Mutation of D991 to E in somatic ACE was found to reduce but not eliminate activity. The mutation resulted in a small decrease in kcat (approximately 3.8-fold) as well as a decrease in affinity for the inhibitor trandolaprilat by about 8-fold.

Other residues near the P1′ site of ACE2, but on the other side of the active site cleft are R273 and F274. The corresponding residues in somatic ACE are Gln and Thr, respectively. If there is a conformational change upon binding of substrates and inhibitors, then these residues would play a significant role in catalysis and substrate recognition. R273 could donate hydrogen bonds to the transition state in a way that resembles the way R127 does in the CPA structure. Thus, R518 and R273 of ACE2 are analogous to R145 and R127 of CPA, thereby resulting in the observed similar substrate specificity (Christianson & Lipscomb, supra).

P2 Substrate Binding Site of ACE2

The residues that are in the vicinity of the P2 binding site of ACE2 can be determined from the above tetra-peptide docking results. These residues are His 379, Glu 402, His 401, Asp 382, Tyr 385, Asn 394 on the Zn side of the active site cleft, and Arg 514 on the opposing side. Of these residues only two are different in somatic ACE: ACE2 amino acid residues D382 and N394 are Phe and Glu in sACE, respectively.

P3 Substrate Binding Site of ACE2

The residues that are in the vicinity of the P2 binding site of ACE2 can be determined from the above tetra-peptide docking results. These residues are Asp 382, Tyr 385, Asn 394, Phe 40, Trp 349, Ser 44, and Thr 347. Of these residues only two are mutated in somatic ACE: D382F and N394E.

Potential Residues Contributing to Transition State Stabilization

Zinc metalloproteases catalyze the hydrolysis of peptide bonds by polarizing a zinc-bound water molecule so that it can act as a nucleophile that attacks the carbonyl group on the scissle peptide bond of the bound substrate. The subsequent transition state that develops is then stabilized through hydrogen bonds donated by neighboring side chains, thereby facilitating the catalytic mechanism. Certain residues at the base of the active site cleft or on the opposing side of the cleft, just across from the zinc binding site, are responsible for the transition state stabilization for ACE2 catalyzed peptide hydrolysis. These residues are R514, Y515, H505, Y510 and R273. These residues are conserved in somatic ACE except for Y510 which is a valine, and R273 which is a glutamine. Based on the docked tetra-peptide model, the conserved Y515 can get close enough to the tetrahedral intermediate. Y515 is about 6 Å from the scissle carbonyl group in this model. This corresponds to Y610 in the flexible loop at the active site of neurolysin.

Site directed mutagenesis experiments for somatic ACE suggested that H1089, which corresponds to H505 in ACE2, was a catalytic residue responsible for transition state stabilization in somatic ACE (Fernandez et al., J. Biol. Chem. 276, pp. 4998-5004 (2001)). If H505 plays an analogous role for ACE2, it would be necessary for this residue to move a distance of about 10 Å to 12 Å to allow it to be close enough to donate hydrogen bonds to stabilize the transition state. In fact all of the proposed transition state stabilizing residues for ACE2 (R514, Y515, H505, Y510 and R273) are too far away from the modeled tetrapeptide in the native ACE2 structure, but could move toward the zinc site with a conformational shift of segments of subdomain II towards subdomain I.

His and Tyr residues are the most common residues recruited by zinc metalloenzymes for transition state stabilization, but carboxypeptidases such as CPA and carboxypeptidase D (CPD) prefer Arg residues for this function (Kim and Lipscomb, Biochemistry 30, pp. 8171-80 (1991) and Christianson & Lipscomb, supra).

### EXAMPLE 9

### ACE2 Structure with Bound Inhibitors Comparison to Other Structures

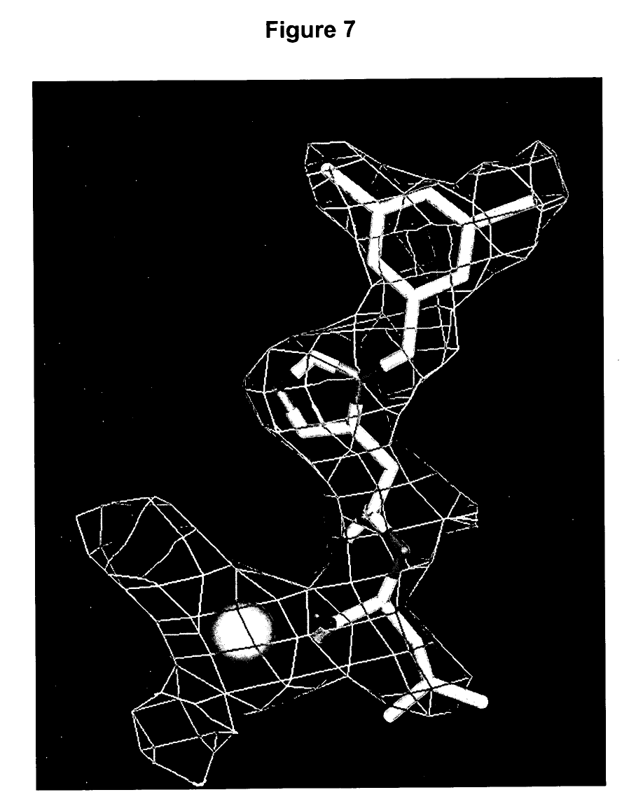

The structure of the extracellular domain of human ACE2 with an inhibitor bound at the active site was solved by molecular replacement to a resolution of 3.3 Å using the native ACE2 structure in the instant invention (see Example 4, FIG. 3A). Refinement statistics for the inhibitor-bound ACE2 structure are shown in Table 3. The bound compound, (S, S)2-{1-carboxy-2-[3-(3,5-dichloro-benzyl)-3H-imidazol-4-yl]-ethylamino}-4-methyl-pentanoic acid (inhibitor1), is a potent inhibitor of human ACE2 with an IC₅₀=0.44 nM, but is a poor inhibitor of tACE (IC₅₀=>100 mM) and carboxypeptidase A (IC₅₀=27 mM) (Dales et al., J. Am. Chem. Soc. 124, pp. 11852-3 (2002)). The structure of the bound (S, S)2-{1-carboxy-2-[3-(3,5-dichloro-benzyl)-3H-imidazol-4-yl]-ethylamino}-4-methyl-pentanoic acid (inhibitor1) is shown in FIG. 7 along with the experimental electron density map near the active site. Despite the lower resolution of the inhibitor-bound structure compared with the native structure, good density was obtained for the inhibitor.

The inhibitor-bound ACE2 structure was further refined to 3.0 Å resolution to yield the structure coordinates provided in FIG. 3B (Table 4 provides refinement statistics). The 3.0 Å structure is nearly identical to the 3.3 Å structure. However, in the 3.0 Å structure, the sidechain ring of amino acid residue His345 swings out in the opposite direction compared to the 3.3 Å structure (FIG. 3A). In this new position, His345 forms a hydrogen bond to the C-terminal carboxylate. The analyses of the complex structure provided below are based on the 3.3 Å structure.

Ligand Dependent Subdomain Hinge Movement:

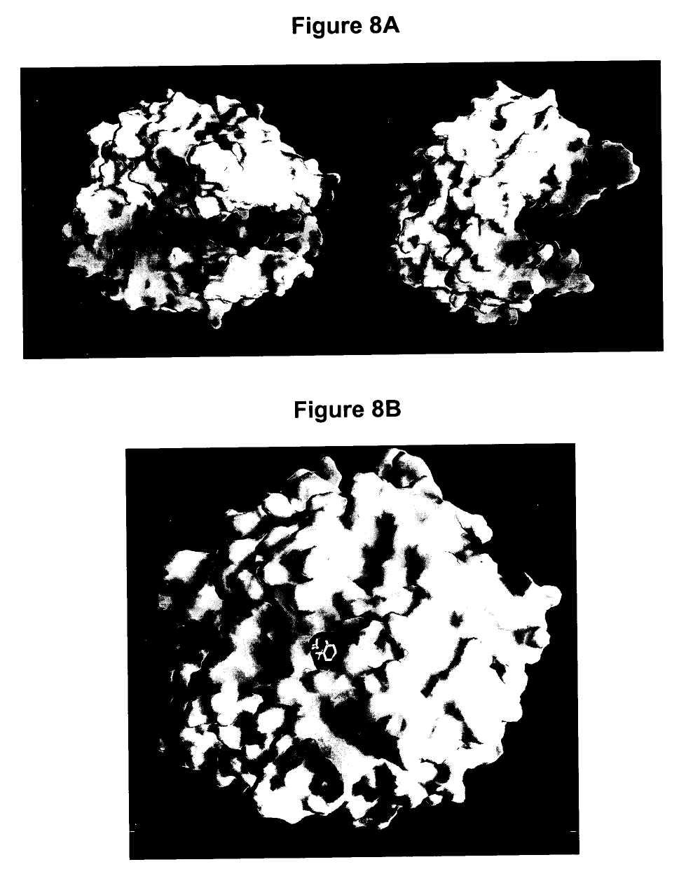

There is a large conformational change that occurs upon binding of the inhibitor, which causes the deep open cleft in the native form of the enzyme (FIGS. 7A and 7B) to close in around the inhibitor. The larger subdomain II, which contains the C-terminal end, remains essentially in the same position as in the native structure, but the other subdomain (containing the zinc ion and the N-terminus) was found to move about 10 Å, essentially mimicking the action of a jaw closing (FIG. 5B). The active site cleft in the native ACE2 structure then becomes essentially closed and resembles a narrow active site tunnel in the inhibitor-bound structure (FIG. 8B).

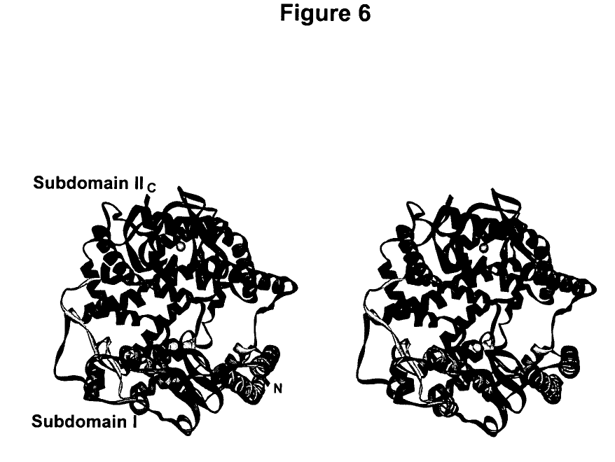

There are distinct regions of the ACE2 enzyme involved in the subdomain movement, specifically Ala 396, Asn 397, Leu 539, His 540, Glu 564, Pro 565 and Trp 566 acting as mechanical hinges with a 22° subdomain rotation upon active site closure (FIG. 6). Subdomain hinge bending motions have been observed for the x-ray structures of other zinc metalloproteases such as thermolysin, P. aeruginosa elastase, B. cereus neutral protease, and astacin, where native and ligand-bound structures were determined (Holland et al., Biochemistry 31, pp. 11310-11316 (1992); Grams et al., Nature Struct. Biol. 3, pp. 671-675 (1996)). The largest previously observed change for these metalloproteases was a 14° hinge bending subdomain motion demonstrated for P. aeruginosa elastase, which resulted in an approximately 2 Å movement to close a N-terminal/C-terminal subdomain gap. Domain closure movements in proteins have been observed for many different classes of enzymes as a common mechanism for the positioning of critical groups around substrates (Gerstein et al., Biochemistry 33, pp. 6739-6749 (1994); Gerstein and Krebs, Nucleic Acids Res. 26, pp. 4280-90 (1998)), and also for the trapping of substrates to prevent escape of reaction intermediates (Knowles, Philos. Trans. R. Soc. London B332, pp. 115-121 (1991)) In this regard, the view of inhibitor1-bound at the active site of ACE2 in FIG. 8B suggests that it may be difficult to get inhibitors (and substrates/products) in and out of the active site of ACE2 without some degree of subdomain hinge flexibility. Many examples of protein flexibility and ligand induced conformational changes in their protein targets have been recently reviewed (Teague, Nature Rev. Drug Discovery 2, pp. 527-541 (2003)).

The lisinopril-bound and native structures of tACE, recently reported by Natesh et al., supra, were found to be nearly identical, suggesting that, unlike ACE2, no ligand dependent conformational change occurs for tACE, or at least under the conditions used to obtain these crystals (pH 4.7 with an unspecified amount of chloride or other anions present). The native ACE2 was crystallized near pH 8.5 with 200 mM MgCl₂ present (400 mM C1⁻). Crystallization at conditions closer to more physiological pH for the native ACE2 structure could explain the difference between the native tACE and native ACE2 structures. The hinge bending equilibrium could be dependent on the pH as well as the concentration of chloride ion ([Cl⁻]) and anion binding equilibrium.

Moreover, sequence differences in the hinge regions of both proteins could also possibly account for the observed differences between homologs in the absence of bound inhibitor. Both the lisinopril-bound and native structures of tACE more closely resemble the inhibitor-bound structure of ACE2 (FIGS. 8A and 8B) rather than the native ACE2 structure. The lisinopril-bound tACE structure can be superimposed onto the inhibitor-bound structure of ACE2 with an RMSD of 1.75 Å (FIGS. 8A and 8B). It should be noted that the lisinopril/tACE structure was obtained by co-crystallization and not by soaking the inhibitor into the native tACE crystals. Soaking of inhibitor1 into native ACE2 crystals always led to destruction of the crystal, presumably due to ligand induced conformational change that accompanies binding. Some element of subdomain hinge bending may also occur for ACE to allow inhibitors (and substrates/products) to enter and exit the active site.

Inhibitor Binding Interactions and Implications for Substrate Specificity and Catalysis:

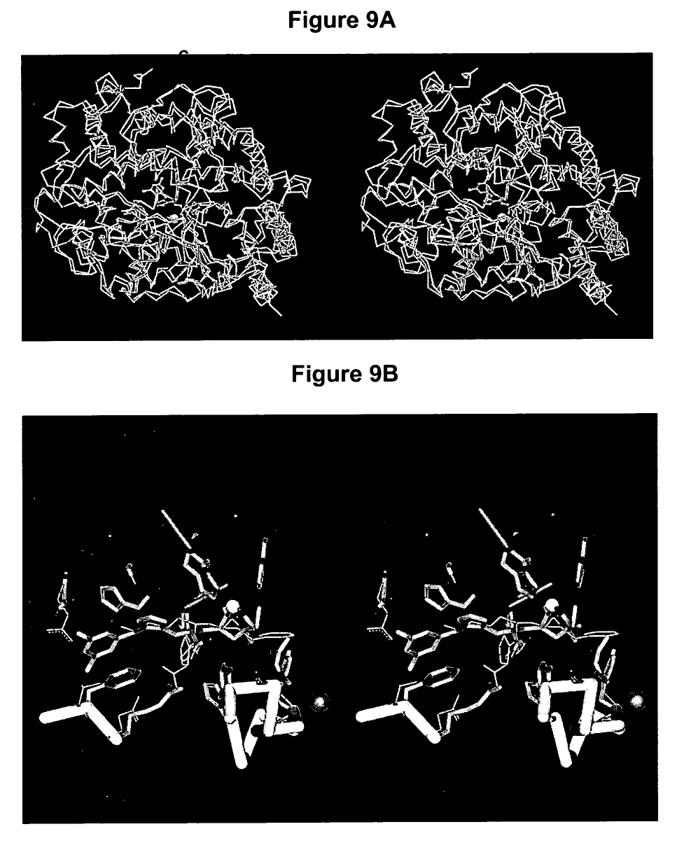

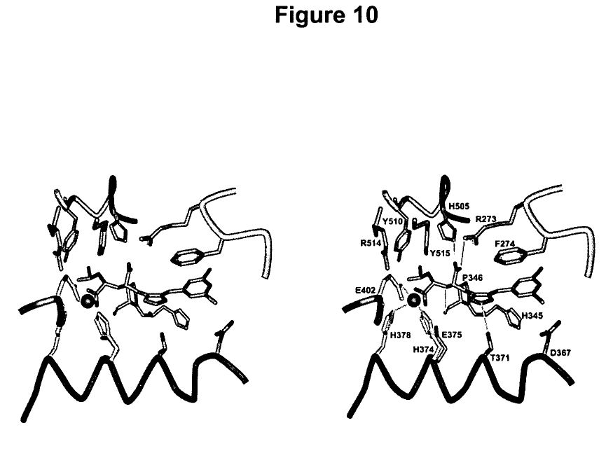

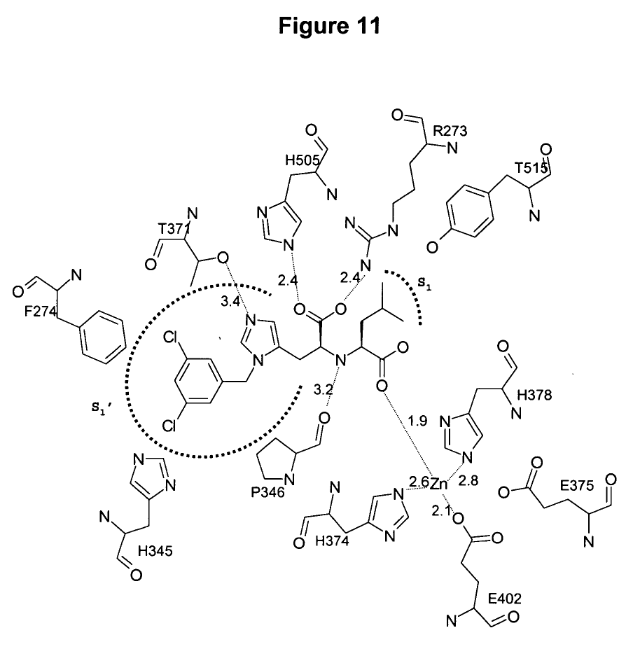

Both subdomains are nearly equally involved in the binding of the potent inhibitor, (S, S)2-{1-carboxy-2-[3-(3,5-dichloro-benzyl)-3H-imidazol-4-yl]-ethylamino}-4-methyl-pentanoic acid (inhibitor1) (FIGS. 8A and 8B). Inspection of the interactions between inhibitor1 and ACE2 reveal important residues responsible for inhibitor binding and presumably for substrate binding and catalysis (FIGS. 9 and 10).

The inhibitor (S, S) 2-{1-carboxy-2-[3-(3,5-dichloro-benzyl)-3H-imidazol-4-yl]-ethylamino}-4-methyl-pentanoic acid (inhibitor1) has two carboxylate groups, one of which binds to the zinc ion displacing the bound water molecule present in the native ACE2 structure. This Zn-bound carboxylate mimics the Zn-bound tetrahedral intermediate that forms after nucleophilic attack of the scissile bound by the zinc-bound water during peptide hydrolysis (Matthews, supra). This tetrahedral intermediate closely resembles the transition state for peptide hydrolysis and is usually stabilized by hydrogen bonds donated by imidazole, phenol, or guanidino functional groups on nearby enzyme side chains (Grams et al., supra; Matthews, supra). For human ACE2, this transition state stabilization most likely occurs through a hydrogen bond donated by the phenolic hydroxyl group of Y515 or R514 (FIGS. 8A, 8B and 9). These residues were 3.8 and 4.1 Å, respectively, from the zinc-bound carboxylate of inhibitor1 in the inhibitor-bound ACE2 structure. These residues are likely to be involved in a true tetrahedral transition state during peptide hydrolysis. Both Y515 and R514 are conserved in tACE as Y523 and R522. In the higher resolution structure of tACE, Y523 was found to be hydrogen bonded to the zinc-bound carboxylate of lisinopril (Kim et al., supra), and R522 was found to bind a chloride ion in tACE. The position of R514 in ACE2 is slightly different than R522 of tACE, presumably due to the absence of this chloride binding site in ACE2 caused by nearby residues that are different between tACE and ACE2 (see below).

S1′ Subsite:

The second carboxylate of inhibitor1 mimics the terminal carboxylate of a peptide substrate and therefore fits into the S1′ subsite of ACE2 (Schechter and Berger, Biochem. Biophys. Res. Commun. 27, pp. 157-162 (1967)). This orientation of the substrate and inhibitor binding in the S1′ subsite is the same orientation reported for inhibitors bound to thermolysin (Holden et al., Biochemistry 26, pp. 8542-8553(1987)) and astacin (Grams et al., supra). Two residues from subdomain 11, R273 and H505 were found to be within hydrogen bonding distance to the terminal carboxylate of inhibitor1. The H505 corresponds to H513 in tACE where it was shown to hydrogen bond to the carbonyl peptide bond between P1′ and P2′ of lisinopril in the inhibitor/tACE structure. Thus, in ACE2 this histidine has the same interaction with inhibitor1 as its corresponding histidine in tACE had with lisinopril (FIGS. 8A, 8B and 9), except that there is no P2′ residue in inhibitor1.

The R273 of ACE2 is changed from Q281 at the equivalent position in tACE and is believed to play an important role in switching the dipeptidyl-peptidase activity of tACE to the observed carboxypeptidase activity of ACE2. Not only does the guanidino group of R273 stabilize the terminal carboxylate of inhibitors and peptide substrates, but its larger size (compared with Gln) also causes steric crowding at any potential S2′ binding site. Indeed, superposition of lisinopril-bound tACE onto the inhibitor1-bound ACE2 (FIGS. 8A and 8B) reveals the guanidino group of R273 to be nearly superimposable on the terminal carboxylate of the S2′ Pro residue of lisinopril, thereby severely limiting the size of the S2′ site in ACE2 compared with tACE.

In addition to R273, there are other residues at the S2′ subsite of ACE2 that differ from tACE and further contribute to the erosion of this subsite in ACE2. The terminal carboxylate of the P2′ Pro residue of lisinopril was shown to be stabilized by hydrogen bond interactions from residues K511, Y520, and Q281 in tACE (Kim et al., supra). These residues correspond to L503, F512, and R273 in ACE2, respectively, thereby eliminating the hydrogen bonds necessary for stabilization of the P2′ position for peptide or inhibitor binding. In addition, the position in human ACE2 that corresponds to T282 of tACE is F274. F274 of human ACE2 has the effect of projecting a large hydrophobic residue into the compromised S2′ subsite of ACE2 (FIGS. 8A and 8B). Together, these changes in ACE2 relative to tACE have the effect of essentially eliminating the S2′ site in ACE2, and suggest an explanation for the observed carboxypeptidase activity of ACE2. The differences at the S1′ and S2′ sites for these two enzymes also presumably explain why the potent ACE inhibitors lisinopril, enalaprilat, and captopril are not active against ACE2 (Tipnis et al., supra).

Residues that line the S1′ site of ACE2 and surround the 3,5-dichloro-benzyl imidazole group of inhibitor1 are: H345 (H353 in tACE), F274 (T282 in tACE), P346 (A354 in tACE), T371 (V380 of tACE), and D367 (E376 in tACE). Of these residues at the S1′ subsite, only H345 is conserved in tACE (H353) where it forms a hydrogen bond between P1′ and P2′ of lisinopril in the inhibitor/tACE structure. The side chain of this conserved histidine is swung about 8 Å out of the way in ACE2 by the stereochemical constraints of the A−>P mutation at the neighboring residue 346. The S1′ subsite in ACE2 is formed by channel between the two subdomains and can accommodate large P1′ residues. There is no limitation on the length of the side chain for residues at the P1′ site since it fits into the channel between the two subdomains. Thus, the very large 3,5-dichloro-benzyl imidazole group of inhibitory fits easily into this S1′ channel, and mirrors the observed preference for large hydrophobic or basic residues at the P1′ position of peptide substrates (Vickers et al., supra)

S1 Subsite:

The S1 subsite of ACE2 appears to be smaller than that observed for tACE. The primary reason is due to the change of V518 of tACE to Y510 in ACE2. In a superposition of the inhibitor-bound ACE2 and the lisinopril-bound tACE structures there is severe steric crowding between the phenolic hydroxyl group of Y510 of ACE2 with the S1 phenylpropyl group of the lisinopril (FIGS. 8A and 8B). The leucyl side chain mimic in inhibitor1 fits very nicely into the S1 site of ACE2, but larger side chains for residues like W, Y, F, R, and K may require some movement of the Y510 side chain. This observation is consistent with the reported substrate specificity data where only medium sized residues such as P, L, and H have been observed at the P1 position. Indeed, peptides with F and Y at the P1 position, such as Angiotensin 1-9 (DRVYIHPFH; SEQ ID NO: 3), Bradykinin (RPPGFSPFR, SEQ ID NO: 7), Leu-enkephalin (YGGFL; SEQ ID NO: 8) Met-enkephalin (YGGFM; SEQ ID NO: 9), and Angiotensin 1-5 (DRVYI; SEQ ID NO: 10) were observed to be inactive as substrates for ACE2 despite the presence of preferred hydrophobic or basic P1′ residues.

This size limitation at the S1 binding site of ACE2 may be another reason why the potent ACE inhibitors, lisinopril and enalaprilat, are not inhibitors of ACE2 since they both have phenylpropyl groups that fit very nicely into the S1 sites of ACE, but result in steric hindrance with Y510 at the bottom of the S1 site of ACE2 (FIGS. 8A and 8B).

Evidence from the substrate screening studies suggested that non-hydrolyzable His-Leu peptidomimetics could inhibit ACE2. The synthesis and optimization of such compounds provided nanomolar ACE2 inhibitors (inhibitor1 and related structures) that are highly selective relative to ACE and CPA. These inhibitors bear two carboxylate functionalities, one for binding the zinc ion, as successfully exploited for ACE inhibitors (Patchett et al., Nature 288, pp. 280-283 (1980)), and a second to mimic the carboxy terminus of a peptide substrate. In the original inhibitor design, the external COOH (Leu) was envisioned to mimic the substrate's C-terminal carboxylate, and the internal COOH (His) was expected to bind to the zinc ion. In this orientation, the isobutyl group would occupy the S₁′ subsite and the substituted histidine would occupy the S1 subsite of ACE2. The potency and Structure Activity Relationship (SAR) of the inhibitors seemed to validate this design principle. However, in the inhibitor-bound crystal structure, inhibitor1 binds in the opposite orientation where the isobutyl group binds in S1 pocket and the 3,5-dichloro-benzyl imidazole group binds in the S₁′ channel. Consequently, the two carboxyl groups bind in an opposite manner as well. Although, the substrate-based design was successful in identifying potent inhibitors, this newly solved, three-dimensional structure allows for further optimization of the ACE2 inhibitors, which may lead to better molecular tools and an enhanced understanding of the enzyme and its function.

Anion Binding Sites:

One chloride ion was found bound to both the native and the inhibitor-bound forms of ACE2. This anion binding site is located in subdomain II and is comprised of three coordinating ligands; K481, R169, and W477. These residues correspond to R489, R186, and W485, respectively, in tACE, which were also found to bind a chloride ion in tACE (CL1 of Natesh et al., supra). In ACE2 this anion binding site is about 21 Å away from the active site zinc ion, and about 13 Å away from the dichlorobenzyl group of the bound inhibitor, (S, S) 2-{1-carboxy-2-[3-(3,5-dichloro-benzyl)-3H-imidazol-4-yl]-ethylamino}-4-methyl-pentanoic acid (inhibitor1). Only this chloride ion binding site could be identified for either the native or inhibitor-bound ACE2 structures.

A second chloride binding site identified in tACE and designated CL2 does not exist in ACE2 because two residues in ACE2 differ from the corresponding residues in tACE structure (P407 tACE->E398 of ACE2 and P519 of tACE->S511 of ACE2). These differences have the effect of projecting Glu and Ser side chains into the location where the chloride ion binds in tACE. Thus, in the inhibitor-bound ACE2 structure, these two residues form a hydrogen bond which takes the place of the CL2 anion binding site of tACE. Due to the greater subdomain separation in the native ACE2 structure, there is a water molecule bound between E398 and S511.

The absence of this second chloride ion binding site in ACE2 is intriguing because the proximity of this second anion binding site to the catalytic zinc ion (approximately 11 Å) in tACE suggested that it played a key role in the anion activation observed for substrate hydrolysis and also inhibitor binding in somatic and testicular ACE (Shapiro and Riordan, Biochemistry 23, pp. 5243-5240 (1984)). This was supported by mutagenesis studies that identified Arg 1098 of sACE (R522 of tACE) as playing a role in anion activation for sACE as well (Liu et al., J. Biol. Chem. 276, pp. 33518-33525 (2001)). The corresponding R522 of tACE was shown to be a ligand to CL2 along with Y224 and a water molecule. However, an anion activation effect on substrate hydrolysis, similar to that of somatic and testicular ACE, has also been described for ACE2 (Vickers et al., supra). Lack of the second anion binding site in ACE2 would suggest a different mechanism responsible for the anion activation effects seen for ACE2. One explanation is that the single chloride ion binding site observed in subdomain II of ACE2 (FIG. 6) is responsible for the anion activation described for ACE2. Another possible explanation is that a second anion binding site does exist in the inhibitor-bound structure of ACE2 but at a different location than that observed for tACE. At the lower resolution of the inhibitor-bound ACE2 structure, it may not be as easy to identify this additional chloride ion. A second chloride binding site was not observed in the 2.2 Å resolution structure of native ACE2.

Proposed Catalytic Mechanism for ACE2 Mediated Peptide Hydrolysis.

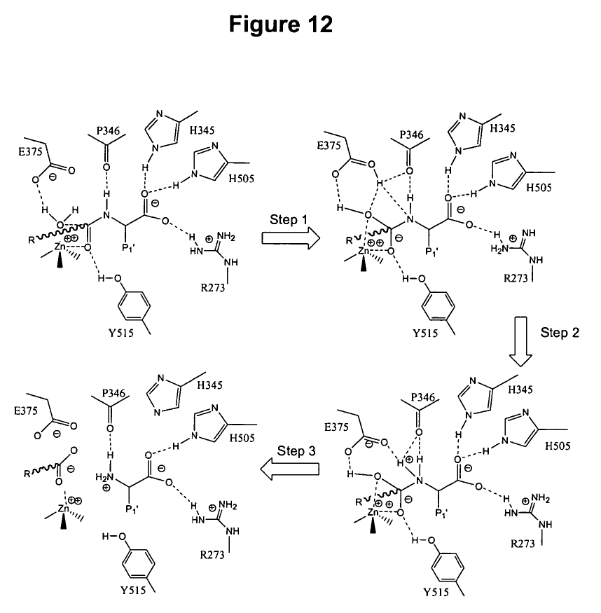

The structural data for native ACE2, taken together with the binding interactions identified for the peptidomimetic inhibitor, (S, S)2-{1-carboxy-2-[3-(3,5-dichloro-benzyl)-3H-imidazol-4-yl]-ethylamino}-4-methyl-pentanoic acid (inhibitor1), at the active site of ACE2 reveals many similarities with the structures of other well characterized HEXXH containing metalloproteases. These structural similarities for residues at the active site are believed to translate into related functional roles. These structural and functional similarities suggest that the catalytic mechanism for ACE2 peptide hydrolysis proceeds in five steps as shown in FIG. 12. The first proposed step involves substrate binding to one subdomain, probably the zinc containing subdomain I followed by a large 22° subdomain hinge bending movement of subdomain I toward subdomain II that closes about a 10 Å gap between these subdomains to bring all the catalytic components into a correct functional orientation. Roughly half the residues important for catalysis are contributed by subdomain I (zinc binding site as well as E375 and P346), and the other half contributed by subdomain II (residues Y515, R273, and H505). Similar subdomain hinge movements have been observed for several other thermolysin-like zinc metalloenzymes but on a smaller scale (Holland et al., supra; Grams et al., supra). These substrate and inhibitor dependent subdomain movements are consistent with induced fit and transition state theories of catalysis (Kraut, Science 242, pp. 533-40 (1988)).

The second step for the proposed catalytic mechanism of ACE2 is the nucleophilic attack of the zinc-bound water molecule at the carbonyl group of the scissile bond. This zinc coordinated water molecule is also hydrogen bonded to Glu 375, thereby providing the means for the enhancement of its nucleophilic role, as described for other zinc metalloproteases (Matthews, supra). Nucleophilic addition transforms the carbonyl group into a tetrahedral intermediate and simultaneously transfers a proton from the attacking water molecule to E375. Y515 of ACE2 is believed to play an important role in stabilizing this tetrahedral intermediate through hydrogen bonding interactions. The phenolic group of Y515 was found to be about 3.8 Å from the zinc-bound inhibitor carboxylate in the inhibitor1-bound ACE2 structure, and is in position for hydrogen bonding to the tetrahedral intermediate. A tyrosine phenolic group plays a similar role for the HEXXH motif containing zinc metalloproteases, thermolysin (Matthews, supra), astacin (Grams et al., supra), and the P. furiosus carboxypeptidase (Arndt et al., supra).

In the third step of the proposed mechanism for ACE2 catalyzed peptide hydrolysis, a proton is transferred from E375 to the leaving nitrogen atom of the scissile bond. For ACE2, the carbonyl group of Pro 346 is positioned to accept a hydrogen bond from this leaving nitrogen atom, as judged from the hydrogen bond observed in inhibitor1-bound structure between this Pro residue and the secondary amine that mimics the P1′ nitrogen of substrates. Thus P346 of ACE2 is believed to play a role in helping to orient the amide nitrogen to accept the E375 proton and stabilize the transition state. Similar roles have been demonstrated for the carbonyl groups of other zinc metalloproteases such as thermolysin (A113), astacin (C64), and P. furiosus carboxypeptidase (P239).

The fourth step in this mechanism is scissile bond cleavage, and step five is a reverse subdomain hinge bending motion to open active site cleft and release of products. This proposed mechanism is similar to other mechanisms proposed for several other well characterized HEXXH containing zinc metallopeptidases such as carboxypeptidase A (Matthews, supra), thermolysin (Matthews, supra), astacin (Grams et al., supra), and the P. furiosus carboxypeptidase (Arndt et al, supra). However, amongst the enzymes ACE2 has the unique property of requiring a much larger hinge to bring all the catalytic components into position.

### EXAMPLE 10

### Inhibitor/Activity Assay

5 μL of 1 mM known peptide substrate (50 μM, final concentration) was added to 45 μL of buffer (50 mM MES, 300 mM NaCl, 10 μM ZnCl₂, and 0.01% Brji-35 at pH 6.5) in a microtiter plate at room temperature. 50 μL of ACE2 at a final concentration of 50 nM was added to initiate the reaction. A simultaneous experiment was done whereby 5 μL of 1 mM known peptide substrate (50 μM, final concentration) and 5 μL of 1 mM suspected inhibitor (50 μM, final concentration) was added to 40 μL of buffer (50 mM MES, 300 mM NaCl, 10 μM ZnCl₂, and 0.01% Brji-35 at pH 6.5) in a microtiter plate at room temperature. 50 μL of ACE2 at a final concentration of 50 nM was added to initiate the reaction (Vickers, et al., supra).

After two hours, the reactions were quenched with 20 μL of 0.5 M EDTA. Reaction products were then analyzed by MALDI-TOF (PerSeptive Biosystems, Framingham, Mass. or equivalent instrument) (Vickers, et al., supra). Comparison of the simultaneous experiments showed the activity of the inhibitor.

While we have described a number of embodiments of this invention, it is apparent that our basic constructions may be altered to provide other embodiments which utilize the products, processes and methods of this invention.

TABLE 1
Heavy Atom Data Statistics

| | Native | | | | |
|---|---|---|---|---|---|
| Derivative | (Zn) | PCMB | HgCl₂ | PIP | K₂PtCl₄ |
| Heavy Atom | Zn | Hg | Hg | Pt | Pt |
| Molarity (mM) | n/a | 1 mM | 1 mM | 1 mM | 1 mM |
| Length of soak | n/a | 3.5 | 30 | 1 | 30 |
| # sites per | 1 | 1 | 1 | 2 | 2 |
| asym. unit^{a} | | | | | |
| wavelength | 1.2824 | 1.009 | 1.009 | 1.072 | 1.072 |
| Unique | 49286 | 41716 | 17421 | 13152 | 14087 |
| Resolution (Å) | 40-2.2 | 30-2.9 | 30-3.0 | 30-3.4 | 30-3.3 |
| Completeness | 96.3 | 96.6 | 90.6 | 95.4 | 94.2 |
| Rsym^{d} | 5.7 | 10.5 | 10.4 | 9.7 | 11.6 |
| Rmerge^{e} | n/a | 21.3 | 37.6 | 20.6 | 21.8 |
| Rcullis^{f} | 0.94 | 0.73 | 0.93 | 0.96 | 0.97 |
| Phasing Power | 1.57 | 1.51 | 0.66 | 0.45 | 0.39 |

^{a}Each asymmetric unit contains one human ACE2 protein.
^{b}Data collected at Brookhaven National Laboratory, NSLS, beamline X25 or Argonne National Laboratory, APS, beamline sector 32, COM-CAT.
^{c}Data include Bivoet pairs.
^{d}R_{sym} = Σ |Iᵢ − Iₘ |/Σ Iₘ where Iᵢ is the intensity of the measured reflection and Iₘ is the mean intensity of all symmetry related reflections.
^{e}R_{merge} = Σ |F_{PH} − F_{P}|/Σ |F_{PH}|
^{f}R_{cullis} = Σ | (F_{PH} ± F_{P}) − F_{H}(calc)/Σ/F_{PH} − F_{P}|
^{g}Phasing Power = F_{H}/E_{RMS} where E_{RMS} is the residual lack of closure.
PCMB refers to para-Chloromercuribenzoate
PIP refers to Di-μ-iodobis (ethylenediamine) diplatinum (II) nitrate

TABLE 2
Native human ACE2 Refinement Statistics^{a}

| | |
|---|---|
| Resolution | 40.0-2.2 |
| | (2.28-2.20) |
| No. reflections | 49286 (3649) |
| Rsym^{b} | 5.7 (40.8) |
| Completeness (%) | 96.3 (47.6) |
| Space Group | C2 |
| a | 103.749 |
| b | 89.590 |
| c | 112.356 |
| β | 109.124 |
| Volume of unit cell | 986854 |
| Solvent Content^{c} | 53% |
| Molecules per asymmetric unit | 1 |
| Reflections used in R_{free} | 4797 (351) |
| No. of protein atoms | 5242 |
| No. of solvent atoms | 298 |
| No. of Zinc atoms | 1 |
| No. of sugar atoms | 42 |
| R_{factor} | 23.7 (39.7) |
| R_{free} | 28.9 (42.1) |
| r.m.s. deviations from ideal | |
| stereochemistry | |
| bond lengths (Å) | 0.008 |
| bond angles (°) | 1.40 |
| dihedrals (°) | 21.5 |
| impropers (°) | 1.05 |
| Mean Bfactor - all atoms (Å³) | 60.4 |

^{a}Numbers in parenthesis represent final shell of data.
^{b}R_{sym} = Σ |Iᵢ − Iₘ|/Σ Iₘ where Iᵢ is the intensity of the measured reflection and Iₘ is the mean intensity of all symmetry related reflections.
^{c}Vₛₒₗᵥₑₙₜ = 1-1.23/Vₘ where Vₘ = volume of protein in the unit cell/volume of unit cell, assuming one molecule per asymmetric unit and four asymmetric units in a monoclinic unit cell.

TABLE 3
Refinement Statistics for 3.3 Å Inhibitor-Bound
ACE2 Structure^{a}.

| | |
|---|---|
| Resolution (Å) | 40.0-3.3 |
| | (3.42-3.30) |
| No. reflections | 39294 (10933) |
| R_{sym}^{b} | 8.1 (18.9) |
| Completeness (%) | 91.9 (89.9) |
| Space Group | C2 |
| a | 100.67 |
| b | 86.78 |
| c | 105.72 |
| β | 103.58 |
| Volume of unit cell (Å³) | 894012 |
| Solvent Content^{c} | 53% |
| Molecules per asymmetric unit. | 1 |
| Reflections used in R_{free} | 1220 |
| No. of protein atoms | 4835 |
| No. of solvent atoms | 76 |
| No. of Zinc atoms | 1 |
| No. of Chloride atoms | 1 |
| No. of sugar atoms | 0 |
| R_{factor} | 22.8% |
| R_{free}^{d} | 33.9% |
| r.m.s. deviations from ideal | |
| stereochemistry | |
| bond lengths (Å) | 0.019 |
| bond angles (°) | 2.16 |
| dihedrals (°) | 23 |
| impropers (°) | 1.2 |
| Mean B_{factor} - all atoms (Å³) | 54.2 |

^{a}Numbers in parenthesis represent final shell of data.
^{b}R_{sym} = Σ |Iᵢ − Iₘ|/Σ Iₘ where Iᵢ is the intensity of the measured reflection and Iₘ is the mean intensity of all symmetry related reflections.
^{c}Vₛₒₗᵥₑₙₜ = 1-1.23/Vₘ where Vₘ = volume of protein in the unit cell/volume of unit cell, assuming one molecule per asymmetric unit and four asymmetric units in a monoclinic unit cell.
^{d}R_{free} is high because of the truncation of the collectrin homology domain (residues 614-740) which results in unaccounted electron density for this C-terminal domain.

TABLE 4
Refinement Statistics for 3.0 Å Inhibitor-Bound
ACE2 Structure^{a}.

| | |
|---|---|
| Resolution (Å) | 40.0-3.0 |
| | (3.08-3.00) |
| No. reflections | 21459 (1041) |
| R_{sym}^{b} | 7.0 (20.4) |
| Completeness (%) | 90.5 (74.5) |
| Space Group | C2 |
| a | 100.5 |
| b | 86.5 |
| c | 105.8 |
| β | 103.6 |
| Volume of unit cell (Å³) | 894383 |
| Solvent Content^{c} | 53% |
| Molecules per asymmetric unit. | 1 |
| Reflections used in R_{free} | 10% |
| No. of protein atoms | 5222 |
| No. of solvent atoms | 15 |
| No. of Zinc atoms | 1 |
| No. of Chloride atoms | 1 |
| No. of sugar atoms | 28 |
| R_{factor} | 24.9% |
| R_{free} | 33.0% |
| r.m.s. deviations from ideal | |
| stereochemistry | |
| bond lengths (Å) | 0.008 |
| bond angles (°) | 1.45 |
| dihedrals (°) | 22.2 |
| impropers (°) | 0.97 |
| Mean B_{factor} - all atoms (Å³) | 76.6 |

^{a}Numbers in parenthesis represent final shell of data.
^{b}R_{sym} = Σ |Iᵢ − Iₘ|/Σ Iₘ where Iᵢ is the intensity of the measured reflection and Iₘ is the mean intensity of all symmetry related reflections.
^{c}Vₛₒₗᵥₑₙₜ = 1-1.23/Vₘ where Vₘ = volume of protein in the unit cell/volume of unit cell, assuming one molecule per asymmetric unit and four asymmetric units in a monoclinic unit cell.

1. A crystal comprising an angiotensin-converting enzyme-related carboxypeptidase or homologue thereof.

2. The crystal according to claim 1, further comprising a chemical entity, wherein said chemical entity binds to the angiotensin-converting enzyme-related carboxypeptidase or homologue thereof.

3. The crystal according to claim 2, wherein the chemical entity binds to the active site on angiotensin-converting enzyme-related carboxypeptidase or homologue thereof.

4. The crystal according to claim 3, wherein the chemical entity is selected from the group consisting of (S,S)2-{1-Carboxy-2-[3-(3,5-dichloro-benzyl)-3H-imidazol-4-yl]-ethylamino}-4-methyl-pentanoic acid, (S,S)2-{1-Carboxy-2-[3-(4-iodo-benzyl)-3H-imidazol-4-yl}-ethylamino}-4-methyl-pentanoic acid, (S,S)2-[2-(6-Bromo-benzothiazol-2-ylcarbamoyl)-1-carboxy-ethylamino]-4-methyl-pentanoic acid and (S, S)2-{1-Carboxy-2-[3-(3,5-dichloro-benzyl)-3H-imidazol-4-yl]-ethylamino}-4-phenyl-butyric acid.

5. The crystal according to claim 3, wherein the chemical entity is (S,S)2-{1-Carboxy-2-[3-(3,5-dichloro-benzyl)-3H-imidazol-4-yl]-ethylamino}-4-methyl-pentanoic acid.

6. The crystal according to claim 1 or 2, wherein said angiotensin-converting enzyme-related carboxypeptidase is selected from the group consisting of amino acid residues 1-740 of human full-length angiotensin-converting enzyme-related carboxypeptidase, amino acid residues 19-740 of human full-length angiotensin-converting enzyme-related carboxypeptidase, amino acid residues 1-611 of human full-length angiotensin-converting enzyme-related carboxypeptidase and amino acid residues 19-611 of human full-length angiotensin-converting enzyme-related carboxypeptidase.

7. The crystal according to claim 1 or 2, wherein said angiotensin-converting enzyme-related carboxypeptidase comprises amino acid residues 19-740 of human full-length angiotensin-converting enzyme-related carboxypeptidase.

8. An isolated, substantially pure, angiotensin-converting enzyme-related carboxypeptidase protein.

9. A crystallizable composition comprising an angiotensin-converting enzyme-related carboxypeptidase or homologue thereof.

10. The crystallizable composition according to claim 9, further comprising a chemical entity.

11. The crystallizable composition according to claim 10, wherein the chemical entity binds to the active site on angiotensin-converting enzyme-related carboxypeptidase or homologue thereof.

12. The crystallizable composition according to claim 11, wherein the chemical entity is selected from the group consisting of (S,S)2-{1-Carboxy-2-[3-(3,5-dichloro-benzyl)-3H-imidazol-4-yl]-ethylamino}-4-methyl-pentanoic acid, (S,S)2-{1-Carboxy-2-[3-(4-iodo-benzyl)-3H-imidazol-4-yl}-ethylamino}-4-methyl-pentanoic acid, (S,S)2-[2-(6-Bromo-benzothiazol-2-ylcarbamoyl)-1-carboxy-ethylamino]-4-methyl-pentanoic acid and (S, S)2-{1-Carboxy-2-[3-(3,5-dichloro-benzyl)-3H-imidazol-4-yl]-ethylamino}-4-phenyl-butyric acid.

13. The crystallizable composition according to claim 11, wherein the chemical entity is (S,S)2-{1-Carboxy-2-[3-(3,5-dichloro-benzyl)-3H-imidazol-4-yl]-ethylamino}-4-methyl-pentanoic acid.

14. The crystallizable composition according to claim 9 or 10, wherein said angiotensin-converting enzyme-related carboxypeptidase is selected from the group consisting of amino acid residues 1-740 of human full-length angiotensin-converting enzyme-related carboxypeptidase, amino acid residues 19-740 of human full-length angiotensin-converting enzyme-related carboxypeptidase, amino acid residues 1-611 human full-length angiotensin-converting enzyme-related carboxypeptidase and amino acid residues 19-611 of human full-length angiotensin-converting enzyme-related carboxypeptidase.

15. The crystallizable composition according to claim 9 or 10, wherein said angiotensin-converting enzyme-related carboxypeptidase comprises amino acid residues 19-740 of human full-length angiotensin-converting enzyme-related carboxypeptidase.

16. A computer comprising:
(a) a machine-readable data storage medium, comprising a data storage material encoded with machine-readable data, wherein said data defines all or part of a binding pocket or protein selected from the group consisting of:
(i) a set of amino acid residues that correspond to human angiotensin-converting enzyme-related carboxypeptidase amino acid residues N149, D269, R273, F274, P346, T371, Y510 and F512 according to FIG. 3A or 3B, wherein the root mean square deviation of the backbone atoms between said amino acid residues and said angiotensin-converting enzyme-related carboxypeptidase amino acid residues is not greater than about 3.0 Å;
(ii) a set of amino acid residues that correspond to human angiotensin-converting enzyme-related carboxypeptidase amino acid residues N149, D269, R273, F274, H345, P346, A348, D367, T371, H364, E375, H378, E402, F504, H505, Y510, F512 and Y515 according to FIG. 3A or 3B, wherein the root mean square deviation of the backbone atoms between said amino acid residues and said angiotensin-converting enzyme-related carboxypeptidase amino acid residues is not greater than about 3.0 Å;
(iii) a set of amino acid residues that correspond to human angiotensin-converting enzyme-related carboxypeptidase amino acid residues N149, D269, R273, F274, H345, P346, A348, D367, T371, H374, E375, H378, E398, E402, R481, L503, F504, H505, Y510, S511, F512, R514, Y515 and E564 according to FIG. 3A or 3B, wherein the root mean square deviation of the backbone atoms between said amino acid residues and said angiotensin-converting enzyme-related carboxypeptidase amino acid residues is not greater than about 3.0 Å; and
(iv) a set of amino acid residues that correspond to human angiotensin-converting enzyme-related carboxypeptidase amino acid residues according to FIG. 1A, 2A, 3A or 3B, wherein the root mean square deviation between said amino acid residues and said angiotensin-converting enzyme-related carboxypeptidase amino acid residues is not more than 1.7 Å;
(b) a working memory for storing instructions for processing said machine-readable data;
(c) a central processing unit coupled to said working memory and to said machine-readable data storage medium for processing said machine-readable data and means for generating three-dimensional structural information of said binding pocket or protein; and
(d) output hardware coupled to said central processing unit for outputting three-dimensional structural information of said binding pocket or protein, or information produced using said three-dimensional structural information of said binding pocket or protein.

17. The computer according to claim 16, wherein the binding pocket is produced by homology modeling of the structure coordinates of said angiotensin-converting enzyme-related carboxypeptidase amino acid residues according to FIG. 1A, 2A, 3A or 3B.

18. The computer according to claim 16, wherein said means for generating three-dimensional structural information is provided by means for generating a three-dimensional graphical representation of said binding pocket or protein.

19. The computer according to claim 16, wherein said output hardware is a display terminal, a printer, CD or DVD recorder, ZIP™ or JAZ™ drive, a disk drive, or other machine-readable data storage device.

20. A method for designing, selecting and/or optimizing a chemical entity that binds to all or part of a binding pocket or protein selected from the group consisting of:
(i) a set of amino acid residues that correspond to human angiotensin-converting enzyme-related carboxypeptidase amino acid residues N149, D269, R273, F274, P346, T371, Y510 and F512 according to FIG. 3A or 3B, wherein the root mean square deviation of the backbone atoms between said amino acid residues and said angiotensin-converting enzyme-related carboxypeptidase amino acid residues is not greater than about 3.0 Å;
(ii) a set of amino acid residues that correspond to human angiotensin-converting enzyme-related carboxypeptidase amino acid residues N149, D269, R273, F274, H345, P346, A348, D367, T371, H364, E375, H378, E402, F504, H505, Y510, F512 and Y515 according to FIG. 3A or 3B, wherein the root mean square deviation of the backbone atoms between said amino acid residues and said angiotensin-converting enzyme-related carboxypeptidase amino acid residues is not greater than about 3.0 Å;
(iii) a set of amino acid residues that correspond to human angiotensin-converting enzyme-related carboxypeptidase amino acid residues N149, D269, R273, F274, H345, P346, A348, D367, T371, H374, E375, H378, E398, E402, R481, L503, F504, H505, Y510, S511, F512, R514, Y515 and E564 according to FIG. 3A or 3B, wherein the root mean square deviation of the backbone atoms between said amino acid residues and said angiotensin-converting enzyme-related carboxypeptidase amino acid residues is not greater than about 3.0 Å; and
(iv) a set of amino acid residues which correspond to human angiotensin-converting enzyme-related carboxypeptidase amino acid residues according to FIG. 1A, 2A, 3A or 3B, wherein the root mean square deviation between said amino acid residues and said human angiotensin-converting enzyme-related carboxypeptidase amino acid residues is not more than 1.7 Å;
comprising the steps of:
(a) providing the structure coordinates of all or part of said binding pocket or protein on a computer comprising the means for generating three-dimensional structural information from said structure coordinates; and
(b) designing, selecting and/or optimizing said chemical entity by performing a fitting operation between said chemical entity and said three-dimensional structural information of all or part of said binding pocket or protein.

21. A method of using a computer for evaluating the ability of a chemical entity to associate with all or part of a binding pocket or protein selected from the group consisting of:
(i) a set of amino acid residues that correspond to human angiotensin-converting enzyme-related carboxypeptidase amino acid residues N149, D269, R273, F274, P346, T371, Y510 and F512 according to FIG. 3A or 3B, wherein the root mean square deviation of the backbone atoms between said amino acid residues and said angiotensin-converting enzyme-related carboxypeptidase amino acid residues is not greater than about 3.0 Å;
(ii) a set of amino acid residues that correspond to human angiotensin-converting enzyme-related carboxypeptidase amino acid residues N149, D269, R273, F274, H345, P346, A348, D367, T371, H364, E375, H378, E402, F504, H505, Y510, F512 and Y515 according to FIG. 3A or 3B, wherein the root mean square deviation of the backbone atoms between said amino acid residues and said angiotensin-converting enzyme-related carboxypeptidase amino acid residues is not greater than about 3.0 Å;
(iii) a set of amino acid residues that correspond to human angiotensin-converting enzyme-related carboxypeptidase amino acid residues N149, D269, R273, F274, H345, P346, A348, D367, T371, H374, E375, H378, E398, E402, R481, L503, F504, H505, Y510, S511, F512, R514, Y515 and E564 according to FIG. 3A or 3B, wherein the root mean square deviation of the backbone atoms between said amino acid residues and said angiotensin-converting enzyme-related carboxypeptidase amino acid residues is not greater than about 3.0 Å; and
(iv) a set of amino acid residues that correspond to human angiotensin-converting enzyme-related carboxypeptidase amino acid residues according to FIG. 1A, 2A, 3A or 3B, wherein the root mean square deviation between said amino acid residues and said angiotensin-converting enzyme-related carboxypeptidase amino acid residues is not more than 1.7 Å;
said method comprising the steps of:
(a) providing the structure coordinates of all or part of said binding pocket or protein on a computer comprising the means for generating three-dimensional structural information from said structure coordinates;
(b) employing computational means to perform a fitting operation between a first chemical entity and all or part of the binding pocket or protein; and
(c) analyzing the results of said fitting operation to quantitate the association between the chemical entity and all or part of the binding pocket or protein.

22. The method according to claim 21, further comprising generating a three-dimensional graphical representation of all or part of the binding pocket or protein prior to step (b).

23. The method according to claim 21, further comprising the steps of:
(d) repeating steps (b) through (c) with a second chemical entity; and
(e) selecting at least one of said first or second chemical entity that associates with said all or part of said binding pocket or protein based on said quantitated association of said first or second chemical entity.

24. A method for identifying an agonist or antagonist of a molecule or molecular complex comprising all or part of a binding pocket or protein selected from the group consisting of:
(i) a set of amino acid residues that correspond to human angiotensin-converting enzyme-related carboxypeptidase amino acid residues N149, D269, R273, F274, P346, T371, Y510 and F512 according to FIG. 3A or 3B, wherein the root mean square deviation of the backbone atoms between said amino acid residues and said angiotensin-converting enzyme-related carboxypeptidase amino acid residues is not greater than about 3.0 Å;
(ii) a set of amino acid residues that correspond to human angiotensin-converting enzyme-related carboxypeptidase amino acid residues N149, D269, R273, F274, H345, P346, A348, D367, T371, H364, E375, H378, E402, F504, H505, Y510, F512 and Y515 according to FIG. 3A or 3B, wherein the root mean square deviation of the backbone atoms between said amino acid residues and said angiotensin-converting enzyme-related carboxypeptidase amino acid residues is not greater than about 3.0 Å;
(iii) a set of amino acid residues that correspond to human angiotensin-converting enzyme-related carboxypeptidase amino acid residues N149, D269, R273, F274, H345, P346, A348, D367, T371, H374, E375, H378, E398, E402, R481, L503, F504, H505, Y510, S511, F512, R514, Y515 and E564 according to FIG. 3A or 3B, wherein the root mean square deviation of the backbone atoms between said amino acid residues and said angiotensin-converting enzyme-related carboxypeptidase amino acid residues is not greater than about 3.0 Å; and
(iv) a set of amino acid residues that correspond to human angiotensin-converting enzyme-related carboxypeptidase amino acid residues according to FIG. 1A, 2A, 3A or 3B, wherein the root mean square deviation between said amino acid residues and said angiotensin-converting enzyme-related carboxypeptidase amino acid residues is not more than 1.7 Å;
comprising the steps of:
(a) using a three-dimensional structure of all or part of the binding pocket or protein of the molecule or molecular complex to design or select a chemical entity;
(b) contacting the chemical entity with the molecule or the molecular complex;
(c) monitoring the catalytic activity of the molecule or molecular complex; and
(d) classifying the chemical entity as an agonist or antagonist based on the effect of the chemical entity on the catalytic activity of the molecule or molecular complex.

25. A method of designing a compound or complex that associates with all or part of a binding pocket selected from the group consisting of:
(i) a set of amino acid residues that correspond to human angiotensin-converting enzyme-related carboxypeptidase amino acid residues N149, D269, R273, F274, P346, T371, Y510 and F512 according to FIG. 3A or 3B, wherein the root mean square deviation of the backbone atoms between said amino acid residues and said angiotensin-converting enzyme-related carboxypeptidase amino acid residues is not greater than about 3.0 Å;
(ii) a set of amino acid residues that correspond to human angiotensin-converting enzyme-related carboxypeptidase amino acid residues N149, D269, R273, F274, H345, P346, A348, D367, T371, H364, E375, H378, E402, F504, H505, Y510, F512 and Y515 according to FIG. 3A or 3B, wherein the root mean square deviation of the backbone atoms between said amino acid residues and said angiotensin-converting enzyme-related carboxypeptidase amino acid residues is not greater than about 3.0 Å; and
(iii) a set of amino acid residues that correspond to human angiotensin-converting enzyme-related carboxypeptidase amino acid residues N149, D269, R273, F274, H345, P346, A348, D367, T371, H374, E375, H378, E398, E402, R481, L503, F504, H505, Y510, S511, F512, R514, Y515 and E564 according to FIG. 3A or 3B, wherein the root mean square deviation of the backbone atoms between said amino acid residues and said angiotensin-converting enzyme-related carboxypeptidase amino acid residues is not greater than about 3.0 Å;
comprising the steps of:
(a) providing the structure coordinates of all or part of said binding pocket on a computer comprising the means for generating three-dimensional structural information from said structure coordinates; and
(b) using the computer to perform a fitting operation to associate a first chemical entity with all or part of the binding pocket;
(c) performing a fitting operation to associate at least a second chemical entity with all or part of the binding pocket;
(d) quantifying the association between the first or second chemical entity and all or part of the binding pocket;
(e) optionally repeating steps (b) to (d) with another first and second chemical entity, selecting a first and a second chemical entity based on said quantified association of all of said first and second chemical entity;
(f) optionally, visually inspecting the relationship of the first and second chemical entity to each other in relation to the binding pocket on a computer screen using the three-dimensional graphical representation of the binding pocket and said first and second chemical entity; and
(g) assembling the first and second chemical entity into a compound or complex that associates with all or part of said binding pocket by model building.

26. A method of utilizing molecular replacement to obtain structural information about a molecule or a molecular complex of unknown structure, comprising the steps of:
(a) crystallizing said molecule or molecular complex;
(b) generating an X-ray diffraction pattern from said crystallized molecule or molecular complex; and
(c) applying at least a portion of the structure coordinates set forth in FIG. 1A, 2A, 3A or 3B or homology model thereof to the X-ray diffraction pattern to generate a three-dimensional electron density map of at least a portion of the molecule or molecular complex whose structure is unknown.

27. The method according to claim 26, wherein the molecule is an angiotensin-converting enzyme-related carboxypeptidase homologue.

28. The method according to claim 26, wherein the molecular complex is selected from the group consisting of an angiotensin-converting enzyme-related carboxypeptidase protein complex and an angiotensin-converting enzyme-related carboxypeptidase homologue complex.
